SEQUENCE LISTING (802 sequences)

SEQ ID NO: 1-208 - skipped (208 entries)

SEQ ID NO: 209 - protein, 8 aa, Artificial Sequence
  Description of Artificial Sequence: Synthetic peptide
GFTFSTYG

SEQ ID NO: 210 - protein, 8 aa, Artificial Sequence
  Description of Artificial Sequence: Synthetic peptide
ISGTGRTT

SEQ ID NO: 211 - protein, 14 aa, Artificial Sequence
  Description of Artificial Sequence: Synthetic peptide
TKERGDYYYGVFDY

SEQ ID NO: 212 - protein, 6 aa, Artificial Sequence
  Description of Artificial Sequence: Synthetic peptide
QTISSW

SEQ ID NO: 213 - protein, 3 aa, Artificial Sequence
  Description of Artificial Sequence: Synthetic peptide
AAS

SEQ ID NO: 214 - protein, 8 aa, Artificial Sequence
  Description of Artificial Sequence: Synthetic peptide
QQYHRSWT

SEQ ID NO: 215 - protein, 121 aa, Artificial Sequence
  Description of Artificial Sequence: Synthetic polypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 216 - protein, 106 aa, Artificial Sequence
  Description of Artificial Sequence: Synthetic polypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 217 - protein, 8 aa, Artificial Sequence
  Description of Artificial Sequence: Synthetic peptide
GFTFSSYW

SEQ ID NO: 218 - protein, 8 aa, Artificial Sequence
  Description of Artificial Sequence: Synthetic peptide
IKEDGSEK

SEQ ID NO: 219 - protein, 15 aa, Artificial Sequence
  Description of Artificial Sequence: Synthetic peptide
AREDYDSYYKYGMDV

SEQ ID NO: 220 - protein, 12 aa, Artificial Sequence
  Description of Artificial Sequence: Synthetic peptide
QSILYSSNNKNY

SEQ ID NO: 221 - protein, 3 aa, Artificial Sequence
  Description of Artificial Sequence: Synthetic peptide
WAS

SEQ ID NO: 222 - protein, 9 aa, Artificial Sequence
  Description of Artificial Sequence: Synthetic peptide
QQYYSTPFT

SEQ ID NO: 223 - protein, 122 aa, Artificial Sequence
  Description of Artificial Sequence: Synthetic polypeptide
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 224 - protein, 113 aa, Artificial Sequence
  Description of Artificial Sequence: Synthetic polypeptide
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 225 - protein, 8 aa, Artificial Sequence
  Description of Artificial Sequence: Synthetic peptide
GGSFTGFY

SEQ ID NO: 226 - protein, 7 aa, Artificial Sequence
  Description of Artificial Sequence: Synthetic peptide
INHRGNT

SEQ ID NO: 227 - protein, 13 aa, Artificial Sequence
  Description of Artificial Sequence: Synthetic peptide
ASPFYDFWSGSDY

SEQ ID NO: 228 - protein, 11 aa, Artificial Sequence
  Description of Artificial Sequence: Synthetic peptide
QSLVHSDGNTY

SEQ ID NO: 229 - protein, 3 aa, Artificial Sequence
  Description of Artificial Sequence: Synthetic peptide
KIS

SEQ ID NO: 230 - protein, 9 aa, Artificial Sequence
  Description of Artificial Sequence: Synthetic peptide
MQATQFPLT

SEQ ID NO: 231 - protein, 119 aa, Artificial Sequence
  Description of Artificial Sequence: Synthetic polypeptide
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 232 - protein, 112 aa, Artificial Sequence
  Description of Artificial Sequence: Synthetic polypeptide
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 233 - protein, 10 aa, Artificial Sequence
  Description of Artificial Sequence: Synthetic peptide
  MOD_RES (3)..(3): P, S, D, Q, or N
  MOD_RES (5)..(5): T or R
  MOD_RES (6)..(6): N, T, Y, or H
GYXFXXYGIS

SEQ ID NO: 234 - protein, 17 aa, Artificial Sequence
  Description of Artificial Sequence: Synthetic peptide
  MOD_RES (4)..(4): T, P, S, or A
  MOD_RES (8)..(8): N, G, V, K, or A
  MOD_RES (9)..(9): T or K
  MOD_RES (10)..(10): H or N
  MOD_RES (13)..(13): Q or R
  MOD_RES (14)..(14): K or M
  MOD_RES (15)..(15): L or H
WISXYNGXXXYAXXXQG

SEQ ID NO: 235 - protein, 15 aa, Artificial Sequence
  Description of Artificial Sequence: Synthetic peptide
  MOD_RES (8)..(8): S or A
  MOD_RES (9)..(9): Y or P
  MOD_RES (15)..(15): V, A, or G
ENYYGSGXXRGGMDX

SEQ ID NO: 236 - protein, 10 aa, Artificial Sequence
  Description of Artificial Sequence: Synthetic peptide
GYDFTYYGIS

SEQ ID NO: 237 - protein, 17 aa, Artificial Sequence
  Description of Artificial Sequence: Synthetic peptide
WISTYNGNTHYARMLQG

SEQ ID NO: 238 - protein, 15 aa, Artificial Sequence
  Description of Artificial Sequence: Synthetic peptide
ENYYGSGAYRGGMDV

SEQ ID NO: 239 - protein, 11 aa, Artificial Sequence
  Description of Artificial Sequence: Synthetic peptide
RASQSVSSYLA

SEQ ID NO: 240 - protein, 7 aa, Artificial Sequence
  Description of Artificial Sequence: Synthetic peptide
DASNRAT

SEQ ID NO: 241 - protein, 9 aa, Artificial Sequence
  Description of Artificial Sequence: Synthetic peptide
QQRSNWPWT

SEQ ID NO: 242 - protein, 124 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 243 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 244 - protein, 453 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 7 aa, Artificial Sequence
  Description of Artificial Sequence: Synthetic peptide
SRSYYWG

SEQ ID NO: 247 - protein, 16 aa, Artificial Sequence
  Description of Artificial Sequence: Synthetic peptide
SIYYNGRTYYNPSLKS

SEQ ID NO: 248 - protein, 11 aa, Artificial Sequence
  Description of Artificial Sequence: Synthetic peptide
EDYGDYGAFDI

SEQ ID NO: 249 - protein, 11 aa, Artificial Sequence
  Description of Artificial Sequence: Synthetic peptide
RASQGISSALA

SEQ ID NO: 250 - protein, 7 aa, Artificial Sequence
  Description of Artificial Sequence: Synthetic peptide
DASSLES

SEQ ID NO: 251 - protein, 9 aa, Artificial Sequence
  Description of Artificial Sequence: Synthetic peptide
QQFNSYPLT

SEQ ID NO: 252 - protein, 121 aa, Artificial Sequence
  Description of Artificial Sequence: Synthetic polypeptide
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 253 - protein, 107 aa, Artificial Sequence [sequence omitted]

SEQ ID NO: 254 - protein, 7 aa, Artificial Sequence
  Description of Artificial Sequence: Synthetic peptide
TSNMGVV

SEQ ID NO: 255 - protein, 16 aa, Artificial Sequence
  Description of Artificial Sequence: Synthetic peptide
HILWDDREYSNPALKS

SEQ ID NO: 256 - protein, 14 aa, Artificial Sequence
  Description of Artificial Sequence: Synthetic peptide
MSRNYYGSSYVMDY

SEQ ID NO: 257 - protein, 10 aa, Artificial Sequence
  Description of Artificial Sequence: Synthetic peptide
SASSSVNYMH

SEQ ID NO: 258 - protein, 7 aa, Artificial Sequence
  Description of Artificial Sequence: Synthetic peptide
STSNLAS

SEQ ID NO: 259 - protein, 8 aa, Artificial Sequence
  Description of Artificial Sequence: Synthetic peptide
HQWNNYGT

SEQ ID NO: 260 - protein, 124 aa, Artificial Sequence
  Description of Artificial Sequence: Synthetic polypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 261 - protein, 106 aa, Artificial Sequence
  Description of Artificial Sequence: Synthetic polypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 262 - protein, 5 aa, Artificial Sequence
  Description of Artificial Sequence: Synthetic peptide
LYGMN

SEQ ID NO: 263 - protein, 5 aa, Artificial Sequence
  Description of Artificial Sequence: Synthetic peptide
NYGMN

SEQ ID NO: 264 - protein, 17 aa, Artificial Sequence
  Description of Artificial Sequence: Synthetic peptide
WINTYTGEPTYADDFKG

SEQ ID NO: 265 - protein, 10 aa, Artificial Sequence
  Description of Artificial Sequence: Synthetic peptide
DTAMDYAMAY

SEQ ID NO: 266 - protein, 13 aa, Artificial Sequence
  Description of Artificial Sequence: Synthetic peptide
DYGKYGDYYAMDY

SEQ ID NO: 267 - protein, 16 aa, Artificial Sequence
  Description of Artificial Sequence: Synthetic peptide
KSSQNIVHSDGNTYLE

SEQ ID NO: 268 - protein, 16 aa, Artificial Sequence
  Description of Artificial Sequence: Synthetic peptide
RSSQSIVHSNGNTYLD

SEQ ID NO: 269 - protein, 7 aa, Artificial Sequence
  Description of Artificial Sequence: Synthetic peptide
KVSNRFS

SEQ ID NO: 270 - protein, 9 aa, Artificial Sequence
  Description of Artificial Sequence: Synthetic peptide
FQGSHVPLT

SEQ ID NO: 271 - protein, 119 aa, Artificial Sequence
  Description of Artificial Sequence: Synthetic polypeptide
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 272 - protein, 119 aa, Artificial Sequence
  Description of Artificial Sequence: Synthetic polypeptide
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 273 - protein, 122 aa, Artificial Sequence
  Description of Artificial Sequence: Synthetic polypeptide
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 274 - protein, 122 aa, Artificial Sequence
  Description of Artificial Sequence: Synthetic polypeptide
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 275 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 276 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 277 - protein, 113 aa, Artificial Sequence
  Description of Artificial Sequence: Synthetic polypeptide
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 278 - protein, 113 aa, Artificial Sequence
  Description of Artificial Sequence: Synthetic polypeptide
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 279 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 280 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 281 - protein, 113 aa, Artificial Sequence
  Description of Artificial Sequence: Synthetic polypeptide
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 282 - protein, 113 aa, Artificial Sequence
  Description of Artificial Sequence: Synthetic polypeptide
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 283 - protein, 10 aa, Artificial Sequence
  Description of Artificial Sequence: Synthetic peptide
GYTFTSSWMH

SEQ ID NO: 284 - protein, 8 aa, Artificial Sequence
  Description of Artificial Sequence: Synthetic peptide
IHPNSGGT

SEQ ID NO: 285 - protein, 14 aa, Artificial Sequence
  Description of Artificial Sequence: Synthetic peptide
ARGDYYGYVSWFAY

SEQ ID NO: 286 - protein, 6 aa, Artificial Sequence
  Description of Artificial Sequence: Synthetic peptide
QNINVL

SEQ ID NO: 287 - protein, 3 aa, Artificial Sequence
  Description of Artificial Sequence: Synthetic peptide
KAS

SEQ ID NO: 288 - protein, 9 aa, Artificial Sequence
  Description of Artificial Sequence: Synthetic peptide
QQGQSYPYT

SEQ ID NO: 289 - protein, 119 aa, Artificial Sequence
  Description of Artificial Sequence: Synthetic polypeptide
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 290 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 291 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 292 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 293 - protein, 121 aa, Artificial Sequence
  Description of Artificial Sequence: Synthetic polypeptide
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 294 - protein, 107 aa, Artificial Sequence
  Description of Artificial Sequence: Synthetic polypeptide
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 295 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 296 - protein, 107 aa, Artificial Sequence
  Description of Artificial Sequence: Synthetic polypeptide
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 297 - protein, 107 aa, Artificial Sequence
  Description of Artificial Sequence: Synthetic polypeptide
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 298 - protein, 10 aa, Artificial Sequence
  Description of Artificial Sequence: Synthetic peptide
GYTFTSYDIN

SEQ ID NO: 299 - protein, 10 aa, Artificial Sequence
  Description of Artificial Sequence: Synthetic peptide
  MOD_RES (8)..(8): N or Y
WLNPNSGXTG

SEQ ID NO: 300 - protein, 10 aa, Artificial Sequence
  Description of Artificial Sequence: Synthetic peptide
EVPETAAFEY

SEQ ID NO: 301 - protein, 14 aa, Artificial Sequence
  Description of Artificial Sequence: Synthetic peptide
  MOD_RES (10)..(10): G or A
  MOD_RES (11)..(11): L, S, or Q
  MOD_RES (14)..(14): H or L
TSSSSDIGAXXGVX

SEQ ID NO: 302 - protein, 7 aa, Artificial Sequence
  Description of Artificial Sequence: Synthetic peptide
GYYNRPS

SEQ ID NO: 303 - protein, 10 aa, Artificial Sequence
  Description of Artificial Sequence: Synthetic peptide
  MOD_RES (3)..(3): Y, W, or F
QSXDGTLSAL

SEQ ID NO: 304 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 305 - protein, 111 aa, Artificial Sequence
  Description of Artificial Sequence: Synthetic polypeptide
  MOD_RES (32)..(32): Any amino acid
  MOD_RES (33)..(33): Any amino acid
  MOD_RES (36)..(36): Any amino acid
  MOD_RES (93)..(93): Any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 306 - protein, 119 aa, Artificial Sequence; identical to SEQ ID NO: 304
  Description of Artificial Sequence: Synthetic polypeptide

SEQ ID NO: 307 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 308 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 309 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 310 - protein, 119 aa, Artificial Sequence; identical to SEQ ID NO: 304
  Description of Artificial Sequence: Synthetic polypeptide

SEQ ID NO: 311 - protein, 111 aa, Artificial Sequence
  Description of Artificial Sequence: Synthetic polypeptide
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 312 - protein, 119 aa, Artificial Sequence; identical to SEQ ID NO: 304
  Description of Artificial Sequence: Synthetic polypeptide

SEQ ID NO: 313 - protein, 111 aa, Artificial Sequence
  Description of Artificial Sequence: Synthetic polypeptide
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 314 - protein, 119 aa, Artificial Sequence; identical to SEQ ID NO: 304
  Description of Artificial Sequence: Synthetic polypeptide

SEQ ID NO: 315 - protein, 111 aa, Artificial Sequence
  Description of Artificial Sequence: Synthetic polypeptide
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 316 - protein, 119 aa, Artificial Sequence; identical to SEQ ID NO: 304
  Description of Artificial Sequence: Synthetic polypeptide

SEQ ID NO: 317 - protein, 111 aa, Artificial Sequence
  Description of Artificial Sequence: Synthetic polypeptide
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 318 - protein, 119 aa, Artificial Sequence; identical to SEQ ID NO: 308
  Description of Artificial Sequence: Synthetic polypeptide

SEQ ID NO: 319 - protein, 111 aa, Artificial Sequence; identical to SEQ ID NO: 307
  Description of Artificial Sequence: Synthetic polypeptide

SEQ ID NO: 320 - protein, 119 aa, Artificial Sequence; identical to SEQ ID NO: 308
  Description of Artificial Sequence: Synthetic polypeptide

SEQ ID NO: 321 - protein, 111 aa, Artificial Sequence; identical to SEQ ID NO: 313
  Description of Artificial Sequence: Synthetic polypeptide

SEQ ID NO: 322 - protein, 119 aa, Artificial Sequence; identical to SEQ ID NO: 308
  Description of Artificial Sequence: Synthetic polypeptide

SEQ ID NO: 323 - protein, 111 aa, Artificial Sequence; identical to SEQ ID NO: 315
  Description of Artificial Sequence: Synthetic polypeptide

SEQ ID NO: 324 - protein, 119 aa, Artificial Sequence; identical to SEQ ID NO: 308
  Description of Artificial Sequence: Synthetic polypeptide

SEQ ID NO: 325 - protein, 111 aa, Artificial Sequence; identical to SEQ ID NO: 317
  Description of Artificial Sequence: Synthetic polypeptide

SEQ ID NO: 326 - protein, 119 aa, Artificial Sequence; identical to SEQ ID NO: 308
  Description of Artificial Sequence: Synthetic polypeptide

SEQ ID NO: 327 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 328 - protein, 5 aa, Artificial Sequence
  Description of Artificial Sequence: Synthetic peptide
SYFWS

SEQ ID NO: 329 - protein, 16 aa, Artificial Sequence
  Description of Artificial Sequence: Synthetic peptide
YIYYSGNTKYNPSLKS

SEQ ID NO: 330 - protein, 10 aa, Artificial Sequence
  Description of Artificial Sequence: Synthetic peptide
ETGSYYGFDY

SEQ ID NO: 331 - protein, 11 aa, Artificial Sequence
  Description of Artificial Sequence: Synthetic peptide
RASQSINNYLN

SEQ ID NO: 332 - protein, 7 aa, Artificial Sequence
  Description of Artificial Sequence: Synthetic peptide
AASSLQS

SEQ ID NO: 333 - protein, 9 aa, Artificial Sequence
  Description of Artificial Sequence: Synthetic peptide
QQSYSTPRT

SEQ ID NO: 334 - protein, 118 aa, Artificial Sequence
  Description of Artificial Sequence: Synthetic polypeptide
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 335 - protein, 107 aa, Artificial Sequence
  Description of Artificial Sequence: Synthetic polypeptide
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 336 - protein, 5 aa, Artificial Sequence
  Description of Artificial Sequence: Synthetic peptide
GYYWN

SEQ ID NO: 337 - protein, 16 aa, Artificial Sequence
  Description of Artificial Sequence: Synthetic peptide
EINHAGNTNYNPSLKS

SEQ ID NO: 338 - protein, 12 aa, Artificial Sequence
  Description of Artificial Sequence: Synthetic peptide
GYCRSTTCYFDY

SEQ ID NO: 339 - protein, 12 aa, Artificial Sequence
  Description of Artificial Sequence: Synthetic peptide
RASQSVRSSYLA

SEQ ID NO: 340 - protein, 7 aa, Artificial Sequence
  Description of Artificial Sequence: Synthetic peptide
GASSRAT

SEQ ID NO: 341 - protein, 8 aa, Artificial Sequence
  Description of Artificial Sequence: Synthetic peptide
QQYGSSPT

SEQ ID NO: 342 - protein, 120 aa, Artificial Sequence
  Description of Artificial Sequence: Synthetic polypeptide
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 343 - protein, 107 aa, Artificial Sequence
  Description of Artificial Sequence: Synthetic polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 344 - protein, 116 aa, Artificial Sequence
  Description of Artificial Sequence: Synthetic polypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 345 - protein, 106 aa, Artificial Sequence
  Description of Artificial Sequence: Synthetic polypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 346 - protein, 10 aa, Artificial Sequence
  Description of Artificial Sequence: Synthetic peptide
GFDIQDTYMH

SEQ ID NO: 347 - protein, 17 aa, Artificial Sequence
  Description of Artificial Sequence: Synthetic peptide
RIDPASGHTKYDPKFQV

SEQ ID NO: 348 - protein, 7 aa, Artificial Sequence
  Description of Artificial Sequence: Synthetic peptide
SGGLPDV

SEQ ID NO: 349 - protein, 10 aa, Artificial Sequence
  Description of Artificial Sequence: Synthetic peptide
RASSSVSYMY

SEQ ID NO: 350 - protein, 7 aa, Artificial Sequence
  Description of Artificial Sequence: Synthetic peptide
ATSNLAS

SEQ ID NO: 351 - protein, 9 aa, Artificial Sequence
  Description of Artificial Sequence: Synthetic peptide
QQWEGNPRT

SEQ ID NO: 352 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 353 - protein, 106 aa, Artificial Sequence
  Description of Artificial Sequence: Synthetic polypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 354 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 355 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 356 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 357 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 358 - protein, 121 aa, Artificial Sequence
  Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYNCARRKDGNYYYAMDYWGQGTSVTVSS

SEQ ID NO: 359 - protein, 121 aa, Artificial Sequence
  Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYYCARRKDGNYYYAMDYWGQGTSVTVSS

SEQ ID NO: 360 - protein, 121 aa, Artificial Sequence
  Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYNCARRKDGNYYYAMDYWGQGTTVTVSS

SEQ ID NO: 361 - protein, 121 aa, Artificial Sequence
  Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYYCARRKDGNYYYAMDYWGQGTTVTVSS

SEQ ID NO: 362 - protein, 121 aa, Artificial Sequence
  Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYNCARRKDGNYYYAMDYWGQGTTVTVSS

SEQ ID NO: 363 - protein, 121 aa, Artificial Sequence
  Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYYCARRKDGNYYYAMDYWGQGTTVTVSS

SEQ ID NO: 364 - protein, 121 aa, Artificial Sequence
  Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYNCARRKDGNYYYAMDYWGQGTTVTVSS

SEQ ID NO: 365 - protein, 121 aa, Artificial Sequence
  Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYYCARRKDGNYYYAMDYWGQGTTVTVSS

SEQ ID NO: 366 - protein, 121 aa, Artificial Sequence
  Description of Artificial Sequence: Synthetic polypeptide
QVQLVQSGSELKKPGASVKVSCKASGFTFTNYAMSWVRQAPGKRLEWVSTITSGGSYIYYLDSVKGRFTISRENAKSTLYLQMNSLRTEDTALYNCARRKDGNYYYAMDYWGQGTTVTVSS

SEQ ID NO: 367 - protein, 121 aa, Artificial Sequence
  Description of Artificial Sequence: Synthetic polypeptide
QVQLVQSGSELKKPGASVKVSCKASGFTFTNYAMSWVRQAPGKRLEWVATITSGGSYIYYLDSVKGRFTISRENAKSTLYLQMNSLRTEDTALYYCARRKDGNYYYAMDYWGQGTTVTVSS

SEQ ID NO: 368 - protein, 121 aa, Artificial Sequence
  Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAVYNCARRKDGNYYYAMDYWGQGTTVTVSS

SEQ ID NO: 369 - protein, 121 aa, Artificial Sequence
  Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAIYYCARRKDGNYYYAMDYWGQGTTVTVSS

SEQ ID NO: 370 - protein, 121 aa, Artificial Sequence
  Description of Artificial Sequence: Synthetic polypeptide
EVMLLQSGAEVKKPGASVKVSCKASGFTFTNYAMSWVRQAPGQRLEWVATITSGGSYIYYLDSVKGRFTISRDNAKSTLHLQMNSLRAEDTAVYYCARRKDGNYYYAMDYWGQGTTVTVSS

SEQ ID NO: 371 - protein, 111 aa, Artificial Sequence
  Description of Artificial Sequence: Synthetic polypeptide
DIVLTQSPASLAVSLGQRATISCRASESVDSYGNSFIHWYQQKAGQPPKLLIYRASNLESGIPARFSGSGSRTDFTLTINPVEADDVATYYCQQSYEDPWTFGGGTKLEIK

SEQ ID NO: 372 - protein, 111 aa, Artificial Sequence
  Description of Artificial Sequence: Synthetic polypeptide
  MOD_RES (108)..(108): Any amino acid
DIVLTQSPATLSLSPGERATLSCRASESVDSYGNSFIHWYQQKPGQPPKLLIYRASNLESGIPARFSGSGSRTDFTLTISSLEPEDFAVYYCQQSYEDPWTFGGGTKXEIK

SEQ ID NO: 373 - protein, 111 aa, Artificial Sequence
  Description of Artificial Sequence: Synthetic polypeptide
  MOD_RES (108)..(108): Any amino acid
DIVLTQSPSSLSASVGDRVTITCRASESVDSYGNSFIHWYQQKPGQPPKLLIYRASNLESGIPARFSGSGSRTDFTLTISSLQPEDFATYYCQQSYEDPWTFGGGTKXEIK

SEQ ID NO: 374 - protein, 111 aa, Artificial Sequence
  Description of Artificial Sequence: Synthetic polypeptide
  MOD_RES (108)..(108): Any amino acid
DIVLTQSPDFQSVTPKEKVTITCRASESVDSYGNSFIHWYQQKPGQPPKLLIYRASNLESGIPARFSGSGSRTDFTLTISSLEAEDAATYYCQQSYEDPWTFGGGTKXEIK

SEQ ID NO: 375 - protein, 111 aa, Artificial Sequence
  Description of Artificial Sequence: Synthetic polypeptide
  MOD_RES (108)..(108): Any amino acid
DIVLTQTPLSLSVTPGQPASISCRASESVDSYGNSFIHWYQQKPGQPPKLLIYRASNLESGIPARFSGSGSRTDFTLKISRVEAEDVGVYYCQQSYEDPWTFGGGTKXEIK

SEQ ID NO: 376 - protein, 5 aa, Artificial Sequence
  Description of Artificial Sequence: Synthetic peptide
TYGMS

SEQ ID NO: 377 - protein, 17 aa, Artificial Sequence
  Description of Artificial Sequence: Synthetic peptide
WMNTYSGVTTYADDFKG

SEQ ID NO: 378 - protein, 13 aa, Artificial Sequence
  Description of Artificial Sequence: Synthetic peptide
EGYVFDDYYATDY

SEQ ID NO: 379 - protein, 16 aa, Artificial Sequence
  Description of Artificial Sequence: Synthetic peptide
RSSQNIVHSDGNTYLE

SEQ ID NO: 380 - protein, 7 aa, Artificial Sequence
  Description of Artificial Sequence: Synthetic peptide
KVSNRFS

SEQ ID NO: 381 - protein, 9 aa, Artificial Sequence
  Description of Artificial Sequence: Synthetic peptide
FQGSHVPLT

SEQ ID NO: 382 - protein, 122 aa, Artificial Sequence
  Description of Artificial Sequence: Synthetic polypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 383 - protein, 112 aa, Artificial Sequence
  Description of Artificial Sequence: Synthetic polypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 384 - protein, 5 aa, Artificial Sequence
  Description of Artificial Sequence: Synthetic peptide
KYDIN

SEQ ID NO: 385 - protein, 17 aa, Artificial Sequence
  Description of Artificial Sequence: Synthetic peptide
WIFPGDGRTDYNEKFKG

SEQ ID NO: 386 - protein, 7 aa, Artificial Sequence
  Description of Artificial Sequence: Synthetic peptide
YGPAMDY

SEQ ID NO: 387 - protein, 16 aa, Artificial Sequence
  Description of Artificial Sequence: Synthetic peptide
RSSQTIVHSNGDTYLD

SEQ ID NO: 388 - protein, 7 aa, Artificial Sequence
  Description of Artificial Sequence: Synthetic peptide
KVSNRFS

SEQ ID NO: 389 - protein, 9 aa, Artificial Sequence
  Description of Artificial Sequence: Synthetic peptide
FQGSHVPYT

SEQ ID NO: 390 - protein, 135 aa, Artificial Sequence
  Description of Artificial Sequence: Synthetic polypeptide
MGWSWVFLFLLSVTAGVHSQVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 391 - protein, 131 aa, Artificial Sequence
  Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 392 - RNA, 23 nt, Unknown
  Description of Unknown: miR-155 sequence
uuaaugcuaaucgugauaggggu

SEQ ID NO: 393 - RNA, 22 nt, Unknown
  Description of Unknown: miR-155 sequence
ggggauagugcuaaucguaauu

SEQ ID NO: 394 - RNA, 21 nt, Unknown
  Description of Unknown: miR-155 sequence
uaaugcauggggugggagagg

SEQ ID NO: 395 - RNA, 21 nt, Unknown
  Description of Unknown: miR-155 sequence
uaaugcguggggugggagagg

SEQ ID NO: 396 - RNA, 22 nt, Unknown
  Description of Unknown: miR-155  sequence
uuaaugcuaaucgugauagggg

SEQ ID NO: 397 - RNA, 22 nt, Unknown
  Description of Unknown: miR-155-3p sequence
cuccuacauauuagcauuaaca

SEQ ID NO: 398 - RNA, 23 nt, Unknown
  Description of Unknown: miR-155-5p sequence
uuaaugcuaaucgugauaggggu

SEQ ID NO: 399 - DNA, 8 bp, Unknown
  Description of Unknown: miR-155 seed sequence
tagcatta

SEQ ID NO: 400 - DNA, 6 bp, Unknown
  Description of Unknown: miR-155 seed sequence
agcatt

SEQ ID NO: 401 - RNA, 11 nt, Unknown
  Description of Unknown: miR-155 seed sequence
uagcauuaaca

SEQ ID NO: 402 - DNA, 6 bp, Unknown
  Description of Unknown: miR-155 seed sequence
gcatta

SEQ ID NO: 403 - RNA, 8 nt, Unknown
  Description of Unknown: miR-155 seed sequence
uaaugcua

SEQ ID NO: 404 - DNA, 8 bp, Unknown
  Description of Unknown: miR-155 seed  sequence
agcattaa

SEQ ID NO: 405 - RNA, 65 nt, Homo sapiens
cuguuaaugcuaaucgugauagggguuuuugccuccaacugacuccuacauauuagcauuaacag

SEQ ID NO: 406 - RNA, 50 nt, Unknown
  Description of Unknown: pre miR-155 sequence
uuaaugcuaaucgugauagggguuuuugccuccaacugacuccuacauau

SEQ ID NO: 407 - RNA, 23 nt, Mus sp.
uuaaugcuaauugugauaggggu

SEQ ID NO: 408 - RNA, 65 nt, Mus sp.
cuguuaaugcuaauugugauagggguuuuggccucugacugacuccuaccuguuagcauuaacag

SEQ ID NO: 409 - RNA, 22 nt, Unknown
  Description of Unknown: modified miR-155 targeting oligo sequence
ccccuaucacgauuagcauuaa

SEQ ID NO: 410 - DNA, 22 bp, Artificial Sequence
  Description of Artificial Sequence: Synthetic oligonucleotide
cccctatcacgattagcattaa

SEQ ID NO: 411 - DNA, 22 bp, Artificial Sequence
  Description of Artificial Sequence: Synthetic oligonucleotide
cccctatcacgattagcattaa

SEQ ID NO: 412 - DNA, 22 bp, Artificial Sequence
  Description of Artificial Sequence: Synthetic oligonucleotide
cccctatcacgattagcattaa

SEQ ID NO: 413 - DNA, 15 bp, Artificial Sequence
  Description of Artificial Sequence: Synthetic oligonucleotide
tcacgattagcatta

SEQ ID NO: 414 - DNA, 15 bp, Artificial Sequence
  Description of Artificial Sequence: Synthetic oligonucleotide
tcacgattagcatta

SEQ ID NO: 415 - DNA, 13 bp, Artificial Sequence
  Description of Artificial Sequence: Synthetic oligonucleotide
acgattagcatta

SEQ ID NO: 416 - DNA, 11 bp, Artificial Sequence
  Description of Artificial Sequence: Synthetic oligonucleotide
gattagcatta

SEQ ID NO: 417 - DNA, 15 bp, Artificial Sequence
  Description of Artificial Sequence: Synthetic oligonucleotide
tcacgattagcatta

SEQ ID NO: 418 - DNA, 15 bp, Artificial Sequence
  Description of Artificial Sequence: Synthetic oligonucleotide
tcacgattagcatta

SEQ ID NO: 419 - DNA, 22 bp, Artificial Sequence
  Description of Artificial Sequence: Synthetic oligonucleotide
cccctatcacgattagcattaa

SEQ ID NO: 420 - DNA, 16 bp, Artificial Sequence
  Description of Artificial Sequence: Synthetic oligonucleotide
tcacgattagcattaa

SEQ ID NO: 421 - DNA, 15 bp, Artificial Sequence
  Description of Artificial Sequence: Synthetic oligonucleotide
tcacgattagcatta

SEQ ID NO: 422 - DNA, 15 bp, Artificial Sequence
  Description of Artificial Sequence: Synthetic oligonucleotide
tcacaattagcatta

SEQ ID NO: 423 - DNA, 15 bp, Artificial Sequence
  Description of Artificial Sequence: Synthetic oligonucleotide
tcaacattagactta

SEQ ID NO: 424 - DNA, 9 bp, Artificial Sequence
  Description of Artificial Sequence: Synthetic oligonucleotide
tatgtagga

SEQ ID NO: 425 - DNA, 9 bp, Artificial Sequence
  Description of Artificial Sequence: Synthetic oligonucleotide
ttagcatta

SEQ ID NO: 426 - DNA, 8 bp, Artificial Sequence
  Description of Artificial Sequence: Synthetic oligonucleotide
tagcatta

SEQ ID NO: 427 - DNA, 7 bp, Artificial Sequence
  Description of Artificial Sequence: Synthetic oligonucleotide
agcatta

SEQ ID NO: 428 - DNA, 9 bp, Artificial Sequence
  Description of Artificial Sequence: Synthetic oligonucleotide
tatgtagga

SEQ ID NO: 429 - DNA, 8 bp, Artificial Sequence
  Description of Artificial Sequence: Synthetic oligonucleotide
atgtagga

SEQ ID NO: 430 - DNA, 7 bp, Artificial Sequence
  Description of Artificial Sequence: Synthetic oligonucleotide
tgtagga

SEQ ID NO: 431 - DNA, 8 bp, Artificial Sequence
  Description of Artificial Sequence: Synthetic oligonucleotide
tagcatta

SEQ ID NO: 432 - DNA, 16 bp, Artificial Sequence
  Description of Artificial Sequence: Synthetic oligonucleotide
atcacgattagcatta

SEQ ID NO: 433 - DNA, 16 bp, Artificial Sequence
  Description of Artificial Sequence: Synthetic oligonucleotide
atcacgattagcatta

SEQ ID NO: 434 - DNA, 16 bp, Artificial Sequence
  Description of Artificial Sequence: Synthetic oligonucleotide
atcacgattagcatta

SEQ ID NO: 435 - DNA, 16 bp, Artificial Sequence
  Description of Artificial Sequence: Synthetic oligonucleotide
atcacgattagcatta

SEQ ID NO: 436 - DNA, 16 bp, Artificial Sequence
  Description of Artificial Sequence: Synthetic oligonucleotide
atcacgattagcatta

SEQ ID NO: 437 - DNA, 16 bp, Artificial Sequence
  Description of Artificial Sequence: Synthetic oligonucleotide
atcacgattagcatta

SEQ ID NO: 438 - DNA, 16 bp, Artificial Sequence
  Description of Artificial Sequence: Synthetic oligonucleotide
atcacgattagcatta

SEQ ID NO: 439 - DNA, 16 bp, Artificial Sequence
  Description of Artificial Sequence: Synthetic oligonucleotide
atcacgattagcatta

SEQ ID NO: 440 - DNA, 16 bp, Artificial Sequence
  Description of Artificial Sequence: Synthetic oligonucleotide
atcacgattagcatta

SEQ ID NO: 441 - DNA, 16 bp, Artificial Sequence
  Description of Artificial Sequence: Synthetic oligonucleotide
atcacgattagcatta

SEQ ID NO: 442 - DNA, 16 bp, Artificial Sequence
  Description of Artificial Sequence: Synthetic oligonucleotide
atcacgattagcatta

SEQ ID NO: 443 - DNA, 16 bp, Artificial Sequence
  Description of Artificial Sequence: Synthetic oligonucleotide
atcacgattagcatta

SEQ ID NO: 444 - DNA, 15 bp, Artificial Sequence
  Description of Artificial Sequence: Synthetic oligonucleotide
tcacgattagcatta

SEQ ID NO: 445 - DNA, 15 bp, Artificial Sequence
  Description of Artificial Sequence: Synthetic oligonucleotide
tcacgattagcatta

SEQ ID NO: 446 - DNA, 15 bp, Artificial Sequence
  Description of Artificial Sequence: Synthetic oligonucleotide
tcacgattagcatta

SEQ ID NO: 447 - DNA, 15 bp, Artificial Sequence
  Description of Artificial Sequence: Synthetic oligonucleotide
tcacgattagcatta

SEQ ID NO: 448 - DNA, 15 bp, Artificial Sequence
  Description of Artificial Sequence: Synthetic oligonucleotide
tcacgattagcatta

SEQ ID NO: 449 - DNA, 15 bp, Artificial Sequence
  Description of Artificial Sequence: Synthetic oligonucleotide
tcacgattagcatta

SEQ ID NO: 450 - DNA, 11 bp, Artificial Sequence
  Description of Artificial Sequence: Synthetic oligonucleotide
gattagcatta

SEQ ID NO: 451 - DNA, 12 bp, Artificial Sequence
  Description of Artificial Sequence: Synthetic oligonucleotide
cgattagcatta

SEQ ID NO: 452 - DNA, 12 bp, Artificial Sequence
  Description of Artificial Sequence: Synthetic oligonucleotide
cgattagcatta

SEQ ID NO: 453 - DNA, 14 bp, Artificial Sequence
  Description of Artificial Sequence: Synthetic oligonucleotide
cacgattagcatta

SEQ ID NO: 454 - DNA, 14 bp, Artificial Sequence
  Description of Artificial Sequence: Synthetic oligonucleotide
cacgattagcatta

SEQ ID NO: 455 - DNA, 15 bp, Artificial Sequence
  Description of Artificial Sequence: Synthetic oligonucleotide
tcacgattagcatta

SEQ ID NO: 456 - DNA, 8 bp, Artificial Sequence
  Description of Artificial Sequence: Synthetic oligonucleotide
tagcatta

SEQ ID NO: 457 - DNA, 14 bp, Artificial Sequence
  Description of Artificial Sequence: Synthetic oligonucleotide
cacgattagcatta

SEQ ID NO: 458 - DNA, 14 bp, Artificial Sequence
  Description of Artificial Sequence: Synthetic oligonucleotide
cacgattagcatta

SEQ ID NO: 459 - DNA, 14 bp, Artificial Sequence
  Description of Artificial Sequence: Synthetic oligonucleotide
cacgattagcatta

SEQ ID NO: 460 - DNA, 14 bp, Artificial Sequence
  Description of Artificial Sequence: Synthetic oligonucleotide
cacgattagcatta

SEQ ID NO: 461 - DNA, 14 bp, Artificial Sequence
  Description of Artificial Sequence: Synthetic oligonucleotide
cacgattagcatta

SEQ ID NO: 462 - DNA, 14 bp, Artificial Sequence
  Description of Artificial Sequence: Synthetic oligonucleotide
cacgattagcatta

SEQ ID NO: 463 - DNA, 14 bp, Artificial Sequence
  Description of Artificial Sequence: Synthetic oligonucleotide
cacgattagcatta

SEQ ID NO: 464 - DNA, 14 bp, Artificial Sequence
  Description of Artificial Sequence: Synthetic oligonucleotide
cacgattagcatta

SEQ ID NO: 465 - DNA, 14 bp, Artificial Sequence
  Description of Artificial Sequence: Synthetic oligonucleotide
cacgattagcatta

SEQ ID NO: 466 - DNA, 14 bp, Artificial Sequence
  Description of Artificial Sequence: Synthetic oligonucleotide
cacgattagcatta

SEQ ID NO: 467 - DNA, 14 bp, Artificial Sequence
  Description of Artificial Sequence: Synthetic oligonucleotide
cacgattagcatta

SEQ ID NO: 468 - DNA, 14 bp, Artificial Sequence
  Description of Artificial Sequence: Synthetic oligonucleotide
cacgattagcatta

SEQ ID NO: 469 - DNA, 14 bp, Artificial Sequence
  Description of Artificial Sequence: Synthetic oligonucleotide
cacgattagcatta

SEQ ID NO: 470 - DNA, 14 bp, Artificial Sequence
  Description of Artificial Sequence: Synthetic oligonucleotide
cacgattagcatta

SEQ ID NO: 471 - DNA, 14 bp, Artificial Sequence
  Description of Artificial Sequence: Synthetic oligonucleotide
cacgattagcatta

SEQ ID NO: 472 - DNA, 14 bp, Artificial Sequence
  Description of Artificial Sequence: Synthetic oligonucleotide
cacgattagcatta

SEQ ID NO: 473 - DNA, 14 bp, Artificial Sequence
  Description of Artificial Sequence: Synthetic oligonucleotide
cacgattagcatta

SEQ ID NO: 474 - DNA, 14 bp, Artificial Sequence
  Description of Artificial Sequence: Synthetic oligonucleotide
cacgattagcatta

SEQ ID NO: 475 - DNA, 14 bp, Artificial Sequence
  Description of Artificial Sequence: Synthetic oligonucleotide
cacgattagcatta

SEQ ID NO: 476 - DNA, 14 bp, Artificial Sequence
  Description of Artificial Sequence: Synthetic oligonucleotide
cacgattagcatta

SEQ ID NO: 477 - DNA, 14 bp, Artificial Sequence
  Description of Artificial Sequence: Synthetic oligonucleotide
cacgattagcatta

SEQ ID NO: 478 - DNA, 14 bp, Artificial Sequence
  Description of Artificial Sequence: Synthetic oligonucleotide
cacgattagcatta

SEQ ID NO: 479 - DNA, 14 bp, Artificial Sequence
  Description of Artificial Sequence: Synthetic oligonucleotide
cacgattagcatta

SEQ ID NO: 480 - DNA, 14 bp, Artificial Sequence
  Description of Artificial Sequence: Synthetic oligonucleotide
cacgattagcatta

SEQ ID NO: 481 - DNA, 14 bp, Artificial Sequence
  Description of Artificial Sequence: Synthetic oligonucleotide
cacgattagcatta

SEQ ID NO: 482 - DNA, 13 bp, Artificial Sequence
  Description of Artificial Sequence: Synthetic oligonucleotide
acgattagcatta

SEQ ID NO: 483 - DNA, 12 bp, Artificial Sequence
  Description of Artificial Sequence: Synthetic oligonucleotide
cgattagcatta

SEQ ID NO: 484 - DNA, 11 bp, Artificial Sequence
  Description of Artificial Sequence: Synthetic oligonucleotide
gattagcatta

SEQ ID NO: 485 - DNA, 13 bp, Artificial Sequence
  Description of Artificial Sequence: Synthetic oligonucleotide
acgattagcatta

SEQ ID NO: 486 - DNA, 13 bp, Artificial Sequence
  Description of Artificial Sequence: Synthetic oligonucleotide
acgattagcatta

SEQ ID NO: 487 - DNA, 13 bp, Artificial Sequence
  Description of Artificial Sequence: Synthetic oligonucleotide
acgattagcatta

SEQ ID NO: 488 - DNA, 13 bp, Artificial Sequence
  Description of Artificial Sequence: Synthetic oligonucleotide
acgattagcatta

SEQ ID NO: 489 - DNA, 13 bp, Artificial Sequence
  Description of Artificial Sequence: Synthetic oligonucleotide
acgattagcatta

SEQ ID NO: 490 - DNA, 13 bp, Artificial Sequence
  Description of Artificial Sequence: Synthetic oligonucleotide
acgattagcatta

SEQ ID NO: 491 - DNA, 13 bp, Artificial Sequence
  Description of Artificial Sequence: Synthetic oligonucleotide
acgattagcatta

SEQ ID NO: 492 - DNA, 13 bp, Artificial Sequence
  Description of Artificial Sequence: Synthetic oligonucleotide
acgattagcatta

SEQ ID NO: 493 - DNA, 13 bp, Artificial Sequence
  Description of Artificial Sequence: Synthetic oligonucleotide
acgattagcatta

SEQ ID NO: 494 - DNA, 13 bp, Artificial Sequence
  Description of Artificial Sequence: Synthetic oligonucleotide
acgattagcatta

SEQ ID NO: 495 - DNA, 13 bp, Artificial Sequence
  Description of Artificial Sequence: Synthetic oligonucleotide
acgattagcatta

SEQ ID NO: 496 - DNA, 13 bp, Artificial Sequence
  Description of Artificial Sequence: Synthetic oligonucleotide
acgattagcatta

SEQ ID NO: 497 - DNA, 13 bp, Artificial Sequence
  Description of Artificial Sequence: Synthetic oligonucleotide
acgattagcatta

SEQ ID NO: 498 - DNA, 13 bp, Artificial Sequence
  Description of Artificial Sequence: Synthetic oligonucleotide
acgattagcatta

SEQ ID NO: 499 - DNA, 13 bp, Artificial Sequence
  Description of Artificial Sequence: Synthetic oligonucleotide
acgattagcatta

SEQ ID NO: 500 - DNA, 13 bp, Artificial Sequence
  Description of Artificial Sequence: Synthetic oligonucleotide
acgattagcatta

SEQ ID NO: 501 - DNA, 13 bp, Artificial Sequence
  Description of Artificial Sequence: Synthetic oligonucleotide
acgattagcatta

SEQ ID NO: 502 - DNA, 13 bp, Artificial Sequence
  Description of Artificial Sequence: Synthetic oligonucleotide
acgattagcatta

SEQ ID NO: 503 - DNA, 13 bp, Artificial Sequence
  Description of Artificial Sequence: Synthetic oligonucleotide
acgattagcatta

SEQ ID NO: 504 - DNA, 13 bp, Artificial Sequence
  Description of Artificial Sequence: Synthetic oligonucleotide
acgattagcatta

SEQ ID NO: 505 - DNA, 12 bp, Artificial Sequence
  Description of Artificial Sequence: Synthetic oligonucleotide
cgattagcatta

SEQ ID NO: 506 - DNA, 12 bp, Artificial Sequence
  Description of Artificial Sequence: Synthetic oligonucleotide
cgattagcatta

SEQ ID NO: 507 - DNA, 12 bp, Artificial Sequence
  Description of Artificial Sequence: Synthetic oligonucleotide
cgattagcatta

SEQ ID NO: 508 - DNA, 12 bp, Artificial Sequence
  Description of Artificial Sequence: Synthetic oligonucleotide
cgattagcatta

SEQ ID NO: 509 - DNA, 12 bp, Artificial Sequence
  Description of Artificial Sequence: Synthetic oligonucleotide
cgattagcatta

SEQ ID NO: 510 - DNA, 12 bp, Artificial Sequence
  Description of Artificial Sequence: Synthetic oligonucleotide
cgattagcatta

SEQ ID NO: 511 - DNA, 12 bp, Artificial Sequence
  Description of Artificial Sequence: Synthetic oligonucleotide
cgattagcatta

SEQ ID NO: 512 - DNA, 12 bp, Artificial Sequence
  Description of Artificial Sequence: Synthetic oligonucleotide
cgattagcatta

SEQ ID NO: 513 - DNA, 12 bp, Artificial Sequence
  Description of Artificial Sequence: Synthetic oligonucleotide
cgattagcatta

SEQ ID NO: 514 - DNA, 12 bp, Artificial Sequence
  Description of Artificial Sequence: Synthetic oligonucleotide
cgattagcatta

SEQ ID NO: 515 - DNA, 12 bp, Artificial Sequence
  Description of Artificial Sequence: Synthetic oligonucleotide
cgattagcatta

SEQ ID NO: 516 - DNA, 12 bp, Artificial Sequence
  Description of Artificial Sequence: Synthetic oligonucleotide
cgattagcatta

SEQ ID NO: 517 - DNA, 12 bp, Artificial Sequence
  Description of Artificial Sequence: Synthetic oligonucleotide
cgattagcatta

SEQ ID NO: 518 - DNA, 12 bp, Artificial Sequence
  Description of Artificial Sequence: Synthetic oligonucleotide
cgattagcatta

SEQ ID NO: 519 - DNA, 12 bp, Artificial Sequence
  Description of Artificial Sequence: Synthetic oligonucleotide
cgattagcatta

SEQ ID NO: 520 - DNA, 12 bp, Artificial Sequence
  Description of Artificial Sequence: Synthetic oligonucleotide
cgattagcatta

SEQ ID NO: 521 - DNA, 12 bp, Artificial Sequence
  Description of Artificial Sequence: Synthetic oligonucleotide
cgattagcatta

SEQ ID NO: 522 - DNA, 12 bp, Artificial Sequence
  Description of Artificial Sequence: Synthetic oligonucleotide
cgattagcatta

SEQ ID NO: 523 - DNA, 11 bp, Artificial Sequence
  Description of Artificial Sequence: Synthetic oligonucleotide
gattagcatta

SEQ ID NO: 524 - DNA, 11 bp, Artificial Sequence
  Description of Artificial Sequence: Synthetic oligonucleotide
gattagcatta

SEQ ID NO: 525 - DNA, 11 bp, Artificial Sequence
  Description of Artificial Sequence: Synthetic oligonucleotide
gattagcatta

SEQ ID NO: 526 - DNA, 11 bp, Artificial Sequence
  Description of Artificial Sequence: Synthetic oligonucleotide
gattagcatta

SEQ ID NO: 527 - DNA, 11 bp, Artificial Sequence
  Description of Artificial Sequence: Synthetic oligonucleotide
gattagcatta

SEQ ID NO: 528 - DNA, 11 bp, Artificial Sequence
  Description of Artificial Sequence: Synthetic oligonucleotide
gattagcatta

SEQ ID NO: 529 - DNA, 11 bp, Artificial Sequence
  Description of Artificial Sequence: Synthetic oligonucleotide
gattagcatta

SEQ ID NO: 530 - DNA, 11 bp, Artificial Sequence
  Description of Artificial Sequence: Synthetic oligonucleotide
gattagcatta

SEQ ID NO: 531 - DNA, 11 bp, Artificial Sequence
  Description of Artificial Sequence: Synthetic oligonucleotide
gattagcatta

SEQ ID NO: 532 - DNA, 11 bp, Artificial Sequence
  Description of Artificial Sequence: Synthetic oligonucleotide
gattagcatta

SEQ ID NO: 533 - DNA, 11 bp, Artificial Sequence
  Description of Artificial Sequence: Synthetic oligonucleotide
gattagcatta

SEQ ID NO: 534 - DNA, 11 bp, Artificial Sequence
  Description of Artificial Sequence: Synthetic oligonucleotide
gattagcatta

SEQ ID NO: 535 - DNA, 11 bp, Artificial Sequence
  Description of Artificial Sequence: Synthetic oligonucleotide
gattagcatta

SEQ ID NO: 536 - DNA, 11 bp, Artificial Sequence
  Description of Artificial Sequence: Synthetic oligonucleotide
gattagcatta

SEQ ID NO: 537 - DNA, 11 bp, Artificial Sequence
  Description of Artificial Sequence: Synthetic oligonucleotide
gattagcatta

SEQ ID NO: 538 - DNA, 11 bp, Artificial Sequence
  Description of Artificial Sequence: Synthetic oligonucleotide
gattagcatta

SEQ ID NO: 539 - DNA, 11 bp, Artificial Sequence
  Description of Artificial Sequence: Synthetic oligonucleotide
gattagcatta

SEQ ID NO: 540 - DNA, 14 bp, Artificial Sequence
  Description of Artificial Sequence: Synthetic oligonucleotide
cacgattagcatta

SEQ ID NO: 541 - DNA, 14 bp, Artificial Sequence
  Description of Artificial Sequence: Synthetic oligonucleotide
cacgattagcatta

SEQ ID NO: 542 - DNA, 14 bp, Artificial Sequence
  Description of Artificial Sequence: Synthetic oligonucleotide
cacgattagcatta

SEQ ID NO: 543 - DNA, 14 bp, Artificial Sequence
  Description of Artificial Sequence: Synthetic oligonucleotide
cacgattagcatta

SEQ ID NO: 544 - DNA, 14 bp, Artificial Sequence
  Description of Artificial Sequence: Synthetic oligonucleotide
cacgattagcatta

SEQ ID NO: 545 - DNA, 14 bp, Artificial Sequence
  Description of Artificial Sequence: Synthetic oligonucleotide
cacgattagcatta

SEQ ID NO: 546 - DNA, 14 bp, Artificial Sequence
  Description of Artificial Sequence: Synthetic oligonucleotide
cacgattagcatta

SEQ ID NO: 547 - DNA, 14 bp, Artificial Sequence
  Description of Artificial Sequence: Synthetic oligonucleotide
cacgattagcatta

SEQ ID NO: 548 - DNA, 14 bp, Artificial Sequence
  Description of Artificial Sequence: Synthetic oligonucleotide
cacgattagcatta

SEQ ID NO: 549 - protein, 16 aa, Artificial Sequence
  Description of Artificial Sequence: Synthetic peptide
NDECELCVNVACTGCL

SEQ ID NO: 550 - protein, 15 aa, Artificial Sequence
  Description of Artificial Sequence: Synthetic peptide
DECELCVNVACTGCL

SEQ ID NO: 551 - protein, 14 aa, Artificial Sequence
  Description of Artificial Sequence: Synthetic peptide
DECELCVNVACTGC

SEQ ID NO: 552 - protein, 14 aa, Artificial Sequence
  Description of Artificial Sequence: Synthetic peptide
ECELCVNVACTGCL

SEQ ID NO: 553 - protein, 13 aa, Artificial Sequence
  Description of Artificial Sequence: Synthetic peptide
ECELCVNVACTGC

SEQ ID NO: 554 - protein, 13 aa, Artificial Sequence
  Description of Artificial Sequence: Synthetic peptide
CELCVNVACTGCL

SEQ ID NO: 555 - protein, 12 aa, Artificial Sequence
  Description of Artificial Sequence: Synthetic peptide
CELCVNVACTGC

SEQ ID NO: 556 - protein, 16 aa, Artificial Sequence
  Description of Artificial Sequence: Synthetic peptide
  MOD_RES (16)..(16): D-amino acid
NDECELCVNVACTGCL

SEQ ID NO: 557 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCL

SEQ ID NO: 558 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (16)..(16): D-amino acid
NDECELCVNVACTGCL

SEQ ID NO: 559 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (16)..(16): D-amino acid
NDECELCVNVACTGCL

SEQ ID NO: 560 - protein, 16 aa, Artificial Sequence
  Description of Artificial Sequence: Synthetic peptide
  MOD_RES (1)..(1): D-amino acid
NDECELCVNVACTGCL

SEQ ID NO: 561 - protein, 16 aa, Artificial Sequence
  Description of Artificial Sequence: Synthetic peptide
  MOD_RES (1)..(1): D-amino acid
  MOD_RES (6)..(6): D-amino acid
  MOD_RES (16)..(16): D-amino acid
NDECELCVNVACTGCL

SEQ ID NO: 562 - protein, 15 aa, Artificial Sequence
  Description of Artificial Sequence: Synthetic peptide
NDECELCVNVACTGC

SEQ ID NO: 563 - protein, 16 aa, Artificial Sequence
  Description of Artificial Sequence: Synthetic peptide
  MOD_RES (16)..(16): D-amino acid
NDECELCVNVACTGCL

SEQ ID NO: 564 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCL

SEQ ID NO: 565 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (16)..(16): D-amino acid
NDECELCVNVACTGCL

SEQ ID NO: 566 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCL

SEQ ID NO: 567 - protein, 16 aa, Artificial Sequence
  Description of Artificial Sequence: Synthetic peptide
  MOD_RES (16)..(16): D-amino acid
NDECELCVNVACTGCL

SEQ ID NO: 568 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCL

SEQ ID NO: 569 - protein, 16 aa, Artificial Sequence
  Description of Artificial Sequence: Synthetic peptide
  MOD_RES (16)..(16): D-amino acid
NDECELCVNVACTGCL

SEQ ID NO: 570 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (16)..(16): D-amino acid
NDECELCVNVACTGCL

SEQ ID NO: 571 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (16)..(16): D-amino acid
NDECELCVNVACTGCL

SEQ ID NO: 572 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (16)..(16): D-amino acid
NDECELCVNVACTGCL

SEQ ID NO: 573 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (16)..(16): D-amino acid
NDECELCVNVACTGCL

SEQ ID NO: 574 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (16)..(16): D-amino acid
NDECELCVNVACTGCL

SEQ ID NO: 575 - protein, 16 aa, Artificial Sequence
  Description of Artificial Sequence: Synthetic peptide
  MOD_RES (1)..(1): D-amino acid
  MOD_RES (16)..(16): D-Nal
NDECELCVNVACTGCX

SEQ ID NO: 576 - protein, 16 aa, Artificial Sequence
  Description of Artificial Sequence: Synthetic peptide
  MOD_RES (1)..(1): D-amino acid
  MOD_RES (8)..(8): AIB
  MOD_RES (10)..(10): AIB
  MOD_RES (16)..(16): D-amino acid
NDECELCXNXACTGCL

SEQ ID NO: 577 - protein, 16 aa, Artificial Sequence
  Description of Artificial Sequence: Synthetic peptide
  MOD_RES (1)..(1): D-amino acid
  MOD_RES (7)..(7): Asp-Lactam
  MOD_RES (15)..(15): Orn
  MOD_RES (16)..(16): D-amino acid
NDECELDVNVACTGXL

SEQ ID NO: 578 - protein, 16 aa, Artificial Sequence
  Description of Artificial Sequence: Synthetic peptide
  MOD_RES (1)..(1): D-amino acid
  MOD_RES (16)..(16): D-amino acid
NDECEYCVNVACTGCL

SEQ ID NO: 579 - protein, 16 aa, Artificial Sequence
  Description of Artificial Sequence: Synthetic peptide
  MOD_RES (1)..(1): D-amino acid
  MOD_RES (16)..(16): D-amino acid
NDECESCVNVACTGCL

SEQ ID NO: 580 - protein, 16 aa, Artificial Sequence
  Description of Artificial Sequence: Synthetic peptide
  MOD_RES (1)..(1): D-amino acid
  MOD_RES (16)..(16): D-amino acid
NDECEYCVNVACTGCL

SEQ ID NO: 581 - protein, 16 aa, Artificial Sequence
  Description of Artificial Sequence: Synthetic peptide
  MOD_RES (1)..(1): D-amino acid
  MOD_RES (16)..(16): D-amino acid
NDECEYCVNVACTGCL

SEQ ID NO: 582 - protein, 16 aa, Artificial Sequence
  Description of Artificial Sequence: Synthetic peptide
  MOD_RES (1)..(1): D-amino acid
  MOD_RES (16)..(16): D-amino acid
NDECEYCVNVACTGCL

SEQ ID NO: 583 - protein, 16 aa, Artificial Sequence
  Description of Artificial Sequence: Synthetic peptide
  MOD_RES (1)..(1): D-amino acid
  MOD_RES (16)..(16): D-amino acid
NDECESCVNVACTGCL

SEQ ID NO: 584 - protein, 16 aa, Artificial Sequence
  Description of Artificial Sequence: Synthetic peptide
  MOD_RES (1)..(1): D-amino acid
  MOD_RES (16)..(16): D-amino acid
NDECESCVNVACTGCL

SEQ ID NO: 585 - protein, 16 aa, Artificial Sequence
  Description of Artificial Sequence: Synthetic peptide
  MOD_RES (1)..(1): D-amino acid
  MOD_RES (16)..(16): D-amino acid
NDECESCVNVACTGCL

SEQ ID NO: 586 - protein, 16 aa, Artificial Sequence
  Description of Artificial Sequence: Synthetic peptide
NDECELCVNVACTGCS

SEQ ID NO: 587 - protein, 16 aa, Artificial Sequence
  Description of Artificial Sequence: Synthetic peptide
NDECELCVNVACTGCS

SEQ ID NO: 588 - protein, 16 aa, Artificial Sequence
  Description of Artificial Sequence: Synthetic peptide
NDECELCVNVACTGCS

SEQ ID NO: 589 - protein, 16 aa, Artificial Sequence
  Description of Artificial Sequence: Synthetic peptide
NDECELCVNVACTGCS

SEQ ID NO: 590 - protein, 16 aa, Artificial Sequence
  Description of Artificial Sequence: Synthetic peptide
  MOD_RES (16)..(16): D-amino acid
NDECELCVNVACTGCS

SEQ ID NO: 591 - protein, 16 aa, Artificial Sequence
  Description of Artificial Sequence: Synthetic peptide
  MOD_RES (16)..(16): D-amino acid
NDECELCVNVACTGCS

SEQ ID NO: 592 - protein, 16 aa, Artificial Sequence
  Description of Artificial Sequence: Synthetic peptide
  MOD_RES (16)..(16): D-amino acid
NDECELCVNVACTGCS

SEQ ID NO: 593 - protein, 16 aa, Artificial Sequence
  Description of Artificial Sequence: Synthetic peptide
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Any amino acid
  MOD_RES (13)..(13): Any amino acid
  MOD_RES (14)..(14): Any natural or unnatural amino acid or amino acid analogue
  MOD_RES (16)..(16): Any natural or unnatural amino acid or amino acid analogue
NDECXXCXXXXCXXCX

SEQ ID NO: 594 - protein, 14 aa, Artificial Sequence
  Description of Artificial Sequence: Synthetic peptide
  MOD_RES (1)..(1): Any natural or unnatural amino acid or amino acid analogue
  MOD_RES (3)..(3): Any natural or unnatural amino acid or amino acid analogue
  MOD_RES (4)..(4): Any natural or unnatural amino acid or amino acid analogue
  MOD_RES (6)..(6): Any natural or unnatural amino acid or amino acid analogue
  MOD_RES (7)..(7): Any natural or unnatural amino acid or amino acid analogue
  MOD_RES (8)..(8): Any natural or unnatural amino acid or amino acid analogue
  MOD_RES (9)..(9): Any natural or unnatural amino acid or amino acid analogue
  MOD_RES (11)..(11): Any natural or unnatural amino acid or amino acid analogue
  MOD_RES (12)..(12): Any natural or unnatural amino acid or amino acid analogue
  MOD_RES (14)..(14): Any natural or unnatural amino acid or amino acid analogue
XCXXCXXXXCXXCX

SEQ ID NO: 595 - protein, 14 aa, Artificial Sequence
  Description of Artificial Sequence: Synthetic peptide
  MOD_RES (1)..(1): Any natural or unnatural amino acid or amino acid analogue
  MOD_RES (2)..(2): Cys, Pen, homocysteine, or 3-mercaptoproline
  MOD_RES (4)..(4): Any natural or unnatural amino acid or amino acid analogue
  MOD_RES (5)..(5): Cys, Pen, homocysteine, or 3-mercaptoproline
  MOD_RES (10)..(10): Cys, Pen, homocysteine, or 3-mercaptoproline
  MOD_RES (13)..(13): Cys, Pen, homocysteine, or 3-mercaptoproline
  MOD_RES (14)..(14): Any natural or unnatural amino acid or amino acid analogue
XXEXXVNVAXTGXX

SEQ ID NO: 596 - protein, 14 aa, Artificial Sequence
  Description of Artificial Sequence: Synthetic peptide
  MOD_RES (1)..(1): Any natural or unnatural amino acid or amino acid analogue
  MOD_RES (2)..(2): Cys, Pen, homocysteine, or 3-mercaptoproline
  MOD_RES (3)..(3): Any natural or unnatural amino acid or amino acid analogue
  MOD_RES (4)..(4): Any natural or unnatural amino acid or amino acid analogue
  MOD_RES (5)..(5): Cys, Pen, homocysteine, or 3-mercaptoproline
  MOD_RES (6)..(6): Any natural or unnatural amino acid or amino acid analogue
  MOD_RES (7)..(7): Any natural or unnatural amino acid or amino acid analogue
  MOD_RES (8)..(8): Any natural or unnatural amino acid or amino acid analogue
  MOD_RES (9)..(9): Any natural or unnatural amino acid or amino acid analogue
  MOD_RES (10)..(10): Cys, Pen, homocysteine, or 3-mercaptoproline
  MOD_RES (11)..(11): Any natural or unnatural amino acid or amino acid analogue
  MOD_RES (12)..(12): Any natural or unnatural amino acid or amino acid analogue
  MOD_RES (13)..(13): Cys, Pen, homocysteine, or 3-mercaptoproline
  MOD_RES (14)..(14): Any natural or unnatural amino acid or amino acid analogue
XXXXXXXXXXXXXX

SEQ ID NO: 597 - protein, 16 aa, Artificial Sequence
  Description of Artificial Sequence: Synthetic peptide
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any natural or unnatural amino acid or amino acid analogue
  MOD_RES (16)..(16): Any natural or unnatural amino acid or amino acid analogue
NDDCXXCXNXXCXXCX

SEQ ID NO: 598 - protein, 16 aa, Artificial Sequence
  Description of Artificial Sequence: Synthetic peptide
  MOD_RES (1)..(1): D-amino acid
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any D-amino acid
  MOD_RES (16)..(16): Any natural or unnatural amino acid or amino acid analogue
NEECXXCXNXXCXXCX

SEQ ID NO: 599 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any D-amino acid
  MOD_RES (16)..(16): Any natural or unnatural amino acid or amino acid analogue
NEDCXXCXNXXCXXCX

SEQ ID NO: 600 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any D-amino acid
  MOD_RES (16)..(16): Any natural or unnatural amino acid or amino acid analogue
NDECXXCXNXXCXXCX

SEQ ID NO: 601 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any D-amino acid
  MOD_RES (16)..(16): Any natural or unnatural amino acid or amino acid analogue
NDECXXCXYXXCXXCX

SEQ ID NO: 602 - protein, 16 aa, Artificial Sequence
  Description of Artificial Sequence: Synthetic peptide
  MOD_RES (1)..(1): D-amino acid
  MOD_RES (2)..(2): D-amino acid
  MOD_RES (3)..(3): D-amino acid
  MOD_RES (5)..(5): Any natural or unnatural amino acid or amino acid analogue
  MOD_RES (6)..(6): Any natural or unnatural amino acid or amino acid analogue
  MOD_RES (8)..(8): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any D-amino acid
  MOD_RES (16)..(16): Any natural or unnatural amino acid or amino acid analogue
NEECXXCXYXXCXXCX

SEQ ID NO: 603 - protein, 14 aa, Artificial Sequence
  Description of Artificial Sequence: Synthetic peptide
  MOD_RES (1)..(1): Any natural or unnatural amino acid or amino acid analogue
  MOD_RES (3)..(3): Any natural or unnatural amino acid or amino acid analogue
  MOD_RES (4)..(4): Any natural or unnatural amino acid or amino acid analogue
  MOD_RES (5)..(5): Any natural or unnatural amino acid or amino acid analogue
  MOD_RES (6)..(6): Any natural or unnatural amino acid or amino acid analogue
  MOD_RES (7)..(7): Any natural or unnatural amino acid or amino acid analogue
  MOD_RES (8)..(8): Any natural or unnatural amino acid or amino acid analogue
  MOD_RES (9)..(9): Any natural or unnatural amino acid or amino acid analogue
  MOD_RES (11)..(11): Any natural or unnatural amino acid or amino acid analogue
  MOD_RES (12)..(12): Any natural or unnatural amino acid or amino acid analogue
  MOD_RES (13)..(13): Any natural or unnatural amino acid or amino acid analogue
  MOD_RES (14)..(14): Any natural or unnatural amino acid or amino acid analogue
XCXXXXXXXCXXXX

SEQ ID NO: 604 - protein, 14 aa, Artificial Sequence
  Description of Artificial Sequence: Synthetic peptide
CCEYCCNPACTGCY

SEQ ID NO: 605 - protein, 13 aa, Artificial Sequence
  Description of Artificial Sequence: Synthetic peptide
CCEYCCNPACTGC

SEQ ID NO: 606 - protein, 14 aa, Artificial Sequence
  Description of Artificial Sequence: Synthetic peptide
CCEYCCNPACTGCY

SEQ ID NO: 607 - protein, 16 aa, Artificial Sequence
  Description of Artificial Sequence: Synthetic peptide
NFCCESCCNPACTGCY

SEQ ID NO: 608 - protein, 16 aa, Artificial Sequence
  Description of Artificial Sequence: Synthetic peptide
NFCCEFCCNPACTGCY

SEQ ID NO: 609 - protein, 14 aa, Artificial Sequence
  Description of Artificial Sequence: Synthetic peptide
  MOD_RES (14)..(14): D-amino acid
CCEYCCNPACTGCY

SEQ ID NO: 610 - protein, 14 aa, Artificial Sequence
  Description of Artificial Sequence: Synthetic peptide
CCEYCCNPACTGCY

SEQ ID NO: 611 - protein, 16 aa, Artificial Sequence
  Description of Artificial Sequence: Synthetic peptide
NFCCETCCNPACTGCY

SEQ ID NO: 612 - protein, 16 aa, Artificial Sequence
  Description of Artificial Sequence: Synthetic peptide
  MOD_RES (16)..(16): D-amino acid
NFCCESCCNPACTGCY

SEQ ID NO: 613 - protein, 16 aa, Artificial Sequence
  Description of Artificial Sequence: Synthetic peptide
  MOD_RES (1)..(1): D-amino acid
NFCCESCCNPACTGCY

SEQ ID NO: 614 - protein, 16 aa, Artificial Sequence
  Description of Artificial Sequence: Synthetic peptide
  MOD_RES (1)..(1): D-amino acid
  MOD_RES (16)..(16): D-amino acid
NFCCESCCNPACTGCY

SEQ ID NO: 615 - protein, 16 aa, Artificial Sequence
  Description of Artificial Sequence: Synthetic peptide
  MOD_RES (16)..(16): D-amino acid
NFCCETCCNPACTGCY

SEQ ID NO: 616 - protein, 16 aa, Artificial Sequence
  Description of Artificial Sequence: Synthetic peptide
  MOD_RES (1)..(1): D-amino acid
NFCCETCCNPACTGCY

SEQ ID NO: 617 - protein, 16 aa, Artificial Sequence
  Description of Artificial Sequence: Synthetic peptide
  MOD_RES (1)..(1): D-amino acid
  MOD_RES (16)..(16): D-amino acid
NFCCETCCNPACTGCY

SEQ ID NO: 618 - protein, 16 aa, Artificial Sequence
  Description of Artificial Sequence: Synthetic peptide
  MOD_RES (16)..(16): D-amino acid
NFCCEFCCNPACTGCY

SEQ ID NO: 619 - protein, 16 aa, Artificial Sequence
  Description of Artificial Sequence: Synthetic peptide
  MOD_RES (1)..(1): D-amino acid
NFCCEFCCNPACTGCY

SEQ ID NO: 620 - protein, 16 aa, Artificial Sequence
  Description of Artificial Sequence: Synthetic peptide
  MOD_RES (1)..(1): D-amino acid
  MOD_RES (16)..(16): D-amino acid
NFCCEFCCNPACTGCY

SEQ ID NO: 621 - protein, 14 aa, Artificial Sequence
  Description of Artificial Sequence: Synthetic peptide
CCEYCCNPACTGCY

SEQ ID NO: 622 - protein, 13 aa, Artificial Sequence
  Description of Artificial Sequence: Synthetic peptide
CCEYCCNPACTGC

SEQ ID NO: 623 - protein, 13 aa, Artificial Sequence
  Description of Artificial Sequence: Synthetic peptide
CCEYCCNPACTGC

SEQ ID NO: 624 - protein, 13 aa, Artificial Sequence
  Description of Artificial Sequence: Synthetic peptide
CCEYCCNPACTGC

SEQ ID NO: 625 - protein, 16 aa, Artificial Sequence
  Description of Artificial Sequence: Synthetic peptide
NFCCESCCNPACTGCY

SEQ ID NO: 626 - protein, 16 aa, Artificial Sequence
  Description of Artificial Sequence: Synthetic peptide
NFCCESCCNPACTGCY

SEQ ID NO: 627 - protein, 16 aa, Artificial Sequence
  Description of Artificial Sequence: Synthetic peptide
NFCCESCCNPACTGCY

SEQ ID NO: 628 - protein, 16 aa, Artificial Sequence
  Description of Artificial Sequence: Synthetic peptide
NFCCEFCCNPACTGCY

SEQ ID NO: 629 - protein, 16 aa, Artificial Sequence
  Description of Artificial Sequence: Synthetic peptide
NFCCEFCCNPACTGCY

SEQ ID NO: 630 - protein, 16 aa, Artificial Sequence
  Description of Artificial Sequence: Synthetic peptide
NFCCEFCCNPACTGCY

SEQ ID NO: 631 - protein, 16 aa, Artificial Sequence
  Description of Artificial Sequence: Synthetic peptide
NFCCEYCCNPACTGCY

SEQ ID NO: 632 - protein, 16 aa, Artificial Sequence
  Description of Artificial Sequence: Synthetic peptide
NFCCEYCCNPACTGCY

SEQ ID NO: 633 - protein, 16 aa, Artificial Sequence
  Description of Artificial Sequence: Synthetic peptide
NFCCEYCCNPACTGCY

SEQ ID NO: 634 - protein, 14 aa, Artificial Sequence
  Description of Artificial Sequence: Synthetic peptide
CCESCCNPACTGCY

SEQ ID NO: 635 - protein, 14 aa, Artificial Sequence
  Description of Artificial Sequence: Synthetic peptide
CCEFCCNPACTGCY

SEQ ID NO: 636 - protein, 13 aa, Artificial Sequence
  Description of Artificial Sequence: Synthetic peptide
CCESCCNPACTGC

SEQ ID NO: 637 - protein, 13 aa, Artificial Sequence
  Description of Artificial Sequence: Synthetic peptide
CCEFCCNPACTGC

SEQ ID NO: 638 - protein, 14 aa, Artificial Sequence
  Description of Artificial Sequence: Synthetic peptide
  MOD_RES (1)..(1): Pen
  MOD_RES (2)..(2): Pen
  MOD_RES (5)..(5): Pen
  MOD_RES (6)..(6): Pen
  MOD_RES (10)..(10): Pen
  MOD_RES (13)..(13): Pen
XXEYXXNPAXTGXY

SEQ ID NO: 639 - protein, 13 aa, Artificial Sequence
  Description of Artificial Sequence: Synthetic peptide
  MOD_RES (1)..(1): Pen
  MOD_RES (2)..(2): Pen
  MOD_RES (5)..(5): Pen
  MOD_RES (6)..(6): Pen
  MOD_RES (10)..(10): Pen
  MOD_RES (13)..(13): Pen
XXEYXXNPAXTGX

SEQ ID NO: 640 - protein, 22 aa, Artificial Sequence
  Description of Artificial Sequence: Synthetic peptide
  MOD_RES (1)..(1): Any natural or unnatural amino acid or amino acid analogue
  MOD_RES (2)..(2): Any natural or unnatural amino acid or amino acid analogue
  MOD_RES (3)..(3): Any natural or unnatural amino acid or amino acid analogue
  MOD_RES (4)..(4): Any natural or unnatural amino acid or amino acid analogue
  MOD_RES (5)..(5): Any natural or unnatural amino acid or amino acid analogue
  MOD_RES (6)..(6): Any natural or unnatural amino acid or amino acid analogue
  MOD_RES (11)..(11): Any natural or unnatural amino acid or amino acid analogue
  MOD_RES (15)..(15): Any natural or unnatural amino acid or amino acid analogue
  MOD_RES (16)..(16): Any natural or unnatural amino acid or amino acid analogue
  MOD_RES (17)..(17): Any natural or unnatural amino acid or amino acid analogue
  MOD_RES (19)..(19): Any natural or unnatural amino acid or amino acid analogue
  MOD_RES (20)..(20): Any natural or unnatural amino acid or amino acid analogue
  MOD_RES (22)..(22): Any natural or unnatural amino acid or amino acid analogue
XXXXXXNYCCXYCCXXXCXXCX

SEQ ID NO: 641 - protein, 22 aa, Artificial Sequence
  Description of Artificial Sequence: Synthetic peptide
  MOD_RES (1)..(1): Any natural or unnatural amino acid or amino acid analogue
  MOD_RES (2)..(2): Any natural or unnatural amino acid or amino acid analogue
  MOD_RES (3)..(3): Any natural or unnatural amino acid or amino acid analogue
  MOD_RES (4)..(4): Any natural or unnatural amino acid or amino acid analogue
  MOD_RES (5)..(5): Any natural or unnatural amino acid or amino acid analogue
  MOD_RES (6)..(6): Any natural or unnatural amino acid or amino acid analogue
  MOD_RES (11)..(11): Any natural or unnatural amino acid or amino acid analogue
  MOD_RES (15)..(15): Any natural or unnatural amino acid or amino acid analogue
  MOD_RES (16)..(16): Any natural or unnatural amino acid or amino acid analogue
  MOD_RES (17)..(17): Any natural or unnatural amino acid or amino acid analogue
  MOD_RES (19)..(19): Any natural or unnatural amino acid or amino acid analogue
  MOD_RES (20)..(20): Any natural or unnatural amino acid or amino acid analogue
  MOD_RES (22)..(22): Any natural or unnatural amino acid or amino acid analogue
XXXXXXNFCCXFCCXXXCXXCX

SEQ ID NO: 642 - protein, 16 aa, Artificial Sequence
  Description of Artificial Sequence: Synthetic peptide
  MOD_RES (5)..(5): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any natural or unnatural amino acid or amino acid analogue
  MOD_RES (16)..(16): Any natural or unnatural amino acid or amino acid analogue
NFCCXFCCXXXCXXCX

SEQ ID NO: 643 - protein, 16 aa, Artificial Sequence
  Description of Artificial Sequence: Synthetic peptide
  MOD_RES (3)..(3): Pen
  MOD_RES (5)..(5): Any natural or unnatural amino acid or amino acid analogue
  MOD_RES (8)..(8): Pen
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (13)..(13): Any natural or unnatural amino acid or amino acid analogue
  MOD_RES (14)..(14): Any natural or unnatural amino acid or amino acid analogue
  MOD_RES (16)..(16): Any natural or unnatural amino acid or amino acid analogue
NFXCXFCXXXXCXXCX

SEQ ID NO: 644 - protein, 16 aa, Artificial Sequence
  Description of Artificial Sequence: Synthetic peptide
  MOD_RES (3)..(3): Cys, Pen, homocysteine, or 3-mercaptoproline
  MOD_RES (4)..(4): Cys, Pen, homocysteine, or 3-mercaptoproline
  MOD_RES (5)..(5): Any natural or unnatural amino acid or amino acid analogue
  MOD_RES (6)..(6): Any natural or unnatural amino acid or amino acid analogue
  MOD_RES (7)..(7): Cys, Pen, homocysteine, or 3-mercaptoproline
  MOD_RES (8)..(8): Cys, Pen, homocysteine, or 3-mercaptoproline
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (12)..(12): Cys, Pen, homocysteine, or 3-mercaptoproline
  MOD_RES (13)..(13): Any natural or unnatural amino acid or amino acid analogue
  MOD_RES (14)..(14): Any natural or unnatural amino acid or amino acid analogue
  MOD_RES (15)..(15): Cys, Pen, homocysteine, or 3-mercaptoproline
  MOD_RES (16)..(16): Any natural or unnatural amino acid or amino acid analogue
NFXXXXXXXXXXXXXX

SEQ ID NO: 645 - protein, 14 aa, Artificial Sequence
  Description of Artificial Sequence: Synthetic peptide
  MOD_RES (1)..(1): Cys, Pen, homocysteine, or 3-mercaptoproline
  MOD_RES (2)..(2): Cys, Pen, homocysteine, or 3-mercaptoproline
  MOD_RES (4)..(4): Any natural or unnatural amino acid or amino acid analogue
  MOD_RES (5)..(5): Cys, Pen, homocysteine, or 3-mercaptoproline
  MOD_RES (6)..(6): Cys, Pen, homocysteine, or 3-mercaptoproline
  MOD_RES (10)..(10): Cys, Pen, homocysteine, or 3-mercaptoproline
  MOD_RES (13)..(13): Cys, Pen, homocysteine, or 3-mercaptoproline
XXEXXXNPAXTGXY

SEQ ID NO: 646 - protein, 13 aa, Artificial Sequence
  Description of Artificial Sequence: Synthetic peptide
  MOD_RES (1)..(1): Cys, Pen, homocysteine, or 3-mercaptoproline
  MOD_RES (2)..(2): Cys, Pen, homocysteine, or 3-mercaptoproline
  MOD_RES (4)..(4): Any natural or unnatural amino acid or amino acid analogue
  MOD_RES (5)..(5): Cys, Pen, homocysteine, or 3-mercaptoproline
  MOD_RES (6)..(6): Cys, Pen, homocysteine, or 3-mercaptoproline
  MOD_RES (10)..(10): Cys, Pen, homocysteine, or 3-mercaptoproline
  MOD_RES (13)..(13): Cys, Pen, homocysteine, or 3-mercaptoproline
XXEXXXNPAXTGX

SEQ ID NO: 647 - protein, 15 aa, Artificial Sequence
  Description of Artificial Sequence: Synthetic peptide
  MOD_RES (1)..(1): Any natural or unnatural amino acid or amino acid analogue
  MOD_RES (2)..(2): Cys, Pen, homocysteine, or 3-mercaptoproline
  MOD_RES (3)..(3): Cys, Pen, homocysteine, or 3-mercaptoproline
  MOD_RES (4)..(4): Any natural or unnatural amino acid or amino acid analogue
  MOD_RES (5)..(5): Any natural or unnatural amino acid or amino acid analogue
  MOD_RES (6)..(6): Cys, Pen, homocysteine, or 3-mercaptoproline
  MOD_RES (7)..(7): Cys, Pen, homocysteine, or 3-mercaptoproline
  MOD_RES (8)..(8): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Cys, Pen, homocysteine, or 3-mercaptoproline
  MOD_RES (12)..(12): Any natural or unnatural amino acid or amino acid analogue
  MOD_RES (13)..(13): Any natural or unnatural amino acid or amino acid analogue
  MOD_RES (14)..(14): Cys, Pen, homocysteine, or 3-mercaptoproline
  MOD_RES (15)..(15): Any natural or unnatural amino acid or amino acid analogue
XXXXXXXXXXXXXXX

SEQ ID NO: 648 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCL

SEQ ID NO: 649 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCS

SEQ ID NO: 650 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCS

SEQ ID NO: 651 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCY

SEQ ID NO: 652 - protein, 16 aa, Artificial Sequence
  Description of Artificial Sequence: Synthetic peptide
  MOD_RES (1)..(1): D-amino acid
  MOD_RES (16)..(16): D-amino acid
NDECELCVNVACTGCY

SEQ ID NO: 653 - protein, 16 aa, Artificial Sequence
  Description of Artificial Sequence: Synthetic peptide
  MOD_RES (1)..(1): PyGlu
  MOD_RES (16)..(16): D-amino acid
EDECELCVNVACTGCL

SEQ ID NO: 654 - protein, 16 aa, Artificial Sequence
  Description of Artificial Sequence: Synthetic peptide
  MOD_RES (1)..(1): PyGlu
EDECELCVNVACTGCL

SEQ ID NO: 655 - protein, 16 aa, Artificial Sequence
  Description of Artificial Sequence: Synthetic peptide
NDECELCVNVACTGCL

SEQ ID NO: 656 - protein, 16 aa, Artificial Sequence
  Description of Artificial Sequence: Synthetic peptide
NDECELCVNVACTGCL

SEQ ID NO: 657 - protein, 16 aa, Artificial Sequence
  Description of Artificial Sequence: Synthetic peptide
NDECELCVNVACTGCL

SEQ ID NO: 658 - protein, 16 aa, Artificial Sequence
  Description of Artificial Sequence: Synthetic peptide
  MOD_RES (1)..(1): Any natural or unnatural amino acid or amino acid analogue
  MOD_RES (2)..(2): Any natural or unnatural amino acid or amino acid analogue
  MOD_RES (3)..(3): Any natural or unnatural amino acid or amino acid analogue
  MOD_RES (4)..(4): Cys, Pen, homocysteine, or 3-mercaptoproline
  MOD_RES (5)..(5): Any natural or unnatural amino acid or amino acid analogue
  MOD_RES (6)..(6): Any natural or unnatural amino acid or amino acid analogue
  MOD_RES (7)..(7): Cys, Pen, homocysteine, or 3-mercaptoproline
  MOD_RES (8)..(8): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (12)..(12): Cys, Pen, homocysteine, or 3-mercaptoproline
  MOD_RES (13)..(13): Any natural or unnatural amino acid or amino acid analogue
  MOD_RES (14)..(14): Any natural or unnatural amino acid or amino acid analogue
  MOD_RES (15)..(15): Cys, Pen, homocysteine, or 3-mercaptoproline
  MOD_RES (16)..(16): Any natural or unnatural amino acid or amino acid analogue
XXXXXXXXXXXXXXXX

SEQ ID NO: 659 - protein, 16 aa, Artificial Sequence
  Description of Artificial Sequence: Synthetic peptide
NDDCELCVNVACTGCL

SEQ ID NO: 660 - protein, 16 aa, Artificial Sequence
  Description of Artificial Sequence: Synthetic peptide
EDDCELCVNVACTGCL

SEQ ID NO: 661 - protein, 16 aa, Artificial Sequence
  Description of Artificial Sequence: Synthetic peptide
EDECELCVNVACTGCL

SEQ ID NO: 662 - protein, 16 aa, Artificial Sequence
  Description of Artificial Sequence: Synthetic peptide
EEDCELCVNVACTGCL

SEQ ID NO: 663 - protein, 16 aa, Artificial Sequence
  Description of Artificial Sequence: Synthetic peptide
EEECELCVNVACTGCL

SEQ ID NO: 664 - protein, 16 aa, Artificial Sequence
  Description of Artificial Sequence: Synthetic peptide
DDDCELCVNVACTGCL

SEQ ID NO: 665 - protein, 16 aa, Artificial Sequence
  Description of Artificial Sequence: Synthetic peptide
DDECELCVNVACTGCL

SEQ ID NO: 666 - protein, 16 aa, Artificial Sequence
  Description of Artificial Sequence: Synthetic peptide
DEDCELCVNVACTGCL

SEQ ID NO: 667 - protein, 16 aa, Artificial Sequence
  Description of Artificial Sequence: Synthetic peptide
DEECELCVNVACTGCL

SEQ ID NO: 668 - protein, 16 aa, Artificial Sequence
  Description of Artificial Sequence: Synthetic peptide
QDDCELCVNVACTGCL

SEQ ID NO: 669 - protein, 16 aa, Artificial Sequence
  Description of Artificial Sequence: Synthetic peptide
QDECELCVNVACTGCL

SEQ ID NO: 670 - protein, 16 aa, Artificial Sequence
  Description of Artificial Sequence: Synthetic peptide
QEDCELCVNVACTGCL

SEQ ID NO: 671 - protein, 16 aa, Artificial Sequence
  Description of Artificial Sequence: Synthetic peptide
QEECELCVNVACTGCL

SEQ ID NO: 672 - protein, 16 aa, Artificial Sequence
  Description of Artificial Sequence: Synthetic peptide
KDDCELCVNVACTGCL

SEQ ID NO: 673 - protein, 16 aa, Artificial Sequence
  Description of Artificial Sequence: Synthetic peptide
KDECELCVNVACTGCL

SEQ ID NO: 674 - protein, 16 aa, Artificial Sequence
  Description of Artificial Sequence: Synthetic peptide
KEDCELCVNVACTGCL

SEQ ID NO: 675 - protein, 16 aa, Artificial Sequence
  Description of Artificial Sequence: Synthetic peptide
KEECELCVNVACTGCL

SEQ ID NO: 676 - protein, 16 aa, Artificial Sequence
  Description of Artificial Sequence: Synthetic peptide
EDDCELCVNVACTGCL

SEQ ID NO: 677 - protein, 16 aa, Artificial Sequence
  Description of Artificial Sequence: Synthetic peptide
EDECELCVNVACTGCL

SEQ ID NO: 678 - protein, 16 aa, Artificial Sequence
  Description of Artificial Sequence: Synthetic peptide
EEDCELCVNVACTGCL

SEQ ID NO: 679 - protein, 16 aa, Artificial Sequence
  Description of Artificial Sequence: Synthetic peptide
EEECELCVNVACTGCL

SEQ ID NO: 680 - protein, 16 aa, Artificial Sequence
  Description of Artificial Sequence: Synthetic peptide
DDDCELCVNVACTGCL

SEQ ID NO: 681 - protein, 16 aa, Artificial Sequence
  Description of Artificial Sequence: Synthetic peptide
DDECELCVNVACTGCL

SEQ ID NO: 682 - protein, 16 aa, Artificial Sequence
  Description of Artificial Sequence: Synthetic peptide
DEDCELCVNVACTGCL

SEQ ID NO: 683 - protein, 16 aa, Artificial Sequence
  Description of Artificial Sequence: Synthetic peptide
QDDCELCVNVACTGCL

SEQ ID NO: 684 - protein, 16 aa, Artificial Sequence
  Description of Artificial Sequence: Synthetic peptide
QDECELCVNVACTGCL

SEQ ID NO: 685 - protein, 16 aa, Artificial Sequence
  Description of Artificial Sequence: Synthetic peptide
QEDCELCVNVACTGCL

SEQ ID NO: 686 - protein, 16 aa, Artificial Sequence
  Description of Artificial Sequence: Synthetic peptide
QEECELCVNVACTGCL

SEQ ID NO: 687 - protein, 16 aa, Artificial Sequence
  Description of Artificial Sequence: Synthetic peptide
KDDCELCVNVACTGCL

SEQ ID NO: 688 - protein, 16 aa, Artificial Sequence
  Description of Artificial Sequence: Synthetic peptide
KDECELCVNVACTGCL

SEQ ID NO: 689 - protein, 16 aa, Artificial Sequence
  Description of Artificial Sequence: Synthetic peptide
KEDCELCVNVACTGCL

SEQ ID NO: 690 - protein, 16 aa, Artificial Sequence
  Description of Artificial Sequence: Synthetic peptide
KEECELCVNVACTGCL

SEQ ID NO: 691 - protein, 16 aa, Artificial Sequence
  Description of Artificial Sequence: Synthetic peptide
EDDCELCINMACTGCL

SEQ ID NO: 692 - protein, 16 aa, Artificial Sequence
  Description of Artificial Sequence: Synthetic peptide
EDECELCINMACTGCL

SEQ ID NO: 693 - protein, 16 aa, Artificial Sequence
  Description of Artificial Sequence: Synthetic peptide
EEDCELCINMACTGCL

SEQ ID NO: 694 - protein, 16 aa, Artificial Sequence
  Description of Artificial Sequence: Synthetic peptide
EEECELCINMACTGCL

SEQ ID NO: 695 - protein, 16 aa, Artificial Sequence
  Description of Artificial Sequence: Synthetic peptide
DDDCELCINMACTGCL

SEQ ID NO: 696 - protein, 16 aa, Artificial Sequence
  Description of Artificial Sequence: Synthetic peptide
DDECELCINMACTGCL

SEQ ID NO: 697 - protein, 16 aa, Artificial Sequence
  Description of Artificial Sequence: Synthetic peptide
DEDCELCINMACTGCL

SEQ ID NO: 698 - protein, 16 aa, Artificial Sequence
  Description of Artificial Sequence: Synthetic peptide
DEECELCINMACTGCL

SEQ ID NO: 699 - protein, 16 aa, Artificial Sequence
  Description of Artificial Sequence: Synthetic peptide
QDDCELCINMACTGCL

SEQ ID NO: 700 - protein, 16 aa, Artificial Sequence
  Description of Artificial Sequence: Synthetic peptide
QDECELCINMACTGCL

SEQ ID NO: 701 - protein, 16 aa, Artificial Sequence
  Description of Artificial Sequence: Synthetic peptide
QEDCELCINMACTGCL

SEQ ID NO: 702 - protein, 16 aa, Artificial Sequence
  Description of Artificial Sequence: Synthetic peptide
QEECELCINMACTGCL

SEQ ID NO: 703 - protein, 16 aa, Artificial Sequence
  Description of Artificial Sequence: Synthetic peptide
KDDCELCINMACTGCL

SEQ ID NO: 704 - protein, 16 aa, Artificial Sequence
  Description of Artificial Sequence: Synthetic peptide
KDECELCINMACTGCL

SEQ ID NO: 705 - protein, 16 aa, Artificial Sequence
  Description of Artificial Sequence: Synthetic peptide
KEDCELCINMACTGCL

SEQ ID NO: 706 - protein, 16 aa, Artificial Sequence
  Description of Artificial Sequence: Synthetic peptide
KEECELCINMACTGCL

SEQ ID NO: 707 - protein, 16 aa, Artificial Sequence
  Description of Artificial Sequence: Synthetic peptide
EDDCELCINMACTGCL

SEQ ID NO: 708 - protein, 16 aa, Artificial Sequence
  Description of Artificial Sequence: Synthetic peptide
EDECELCINMACTGCL

SEQ ID NO: 709 - protein, 16 aa, Artificial Sequence
  Description of Artificial Sequence: Synthetic peptide
EEDCELCINMACTGCL

SEQ ID NO: 710 - protein, 16 aa, Artificial Sequence
  Description of Artificial Sequence: Synthetic peptide
EEECELCINMACTGCL

SEQ ID NO: 711 - protein, 16 aa, Artificial Sequence
  Description of Artificial Sequence: Synthetic peptide
DDDCELCINMACTGCL

SEQ ID NO: 712 - protein, 16 aa, Artificial Sequence
  Description of Artificial Sequence: Synthetic peptide
DDECELCINMACTGCL

SEQ ID NO: 713 - protein, 16 aa, Artificial Sequence
  Description of Artificial Sequence: Synthetic peptide
DEDCELCINMACTGCL

SEQ ID NO: 714 - protein, 16 aa, Artificial Sequence
  Description of Artificial Sequence: Synthetic peptide
DEECELCINMACTGCL

SEQ ID NO: 715 - protein, 16 aa, Artificial Sequence
  Description of Artificial Sequence: Synthetic peptide
QDDCELCINMACTGCL

SEQ ID NO: 716 - protein, 16 aa, Artificial Sequence
  Description of Artificial Sequence: Synthetic peptide
QDECELCINMACTGCL

SEQ ID NO: 717 - protein, 16 aa, Artificial Sequence
  Description of Artificial Sequence: Synthetic peptide
QEDCELCINMACTGCL

SEQ ID NO: 718 - protein, 16 aa, Artificial Sequence
  Description of Artificial Sequence: Synthetic peptide
QEECELCINMACTGCL

SEQ ID NO: 719 - protein, 16 aa, Artificial Sequence
  Description of Artificial Sequence: Synthetic peptide
KDDCELCINMACTGCL

SEQ ID NO: 720 - protein, 16 aa, Artificial Sequence
  Description of Artificial Sequence: Synthetic peptide
KDECELCINMACTGCL

SEQ ID NO: 721 - protein, 16 aa, Artificial Sequence
  Description of Artificial Sequence: Synthetic peptide
KEDCELCINMACTGCL

SEQ ID NO: 722 - protein, 16 aa, Artificial Sequence
  Description of Artificial Sequence: Synthetic peptide
KEECELCINMACTGCL

SEQ ID NO: 723 - protein, 15 aa, Artificial Sequence
  Description of Artificial Sequence: Synthetic peptide
  MOD_RES (1)..(1): Any natural or unnatural amino acid or amino acid analogue
  MOD_RES (2)..(2): Any natural or unnatural amino acid or amino acid analogue
  MOD_RES (3)..(3): Any natural or unnatural amino acid or amino acid analogue
  MOD_RES (4)..(4): Cys, Pen, homocysteine, or 3-mercaptoproline
  MOD_RES (5)..(5): Any natural or unnatural amino acid or amino acid analogue
  MOD_RES (6)..(6): Any natural or unnatural amino acid or amino acid analogue
  MOD_RES (7)..(7): Cys, Pen, homocysteine, or 3-mercaptoproline
  MOD_RES (8)..(8): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (12)..(12): Cys, Pen, homocysteine, or 3-mercaptoproline
  MOD_RES (13)..(13): Any natural or unnatural amino acid or amino acid analogue
  MOD_RES (14)..(14): Any natural or unnatural amino acid or amino acid analogue
  MOD_RES (15)..(15): Cys, Pen, homocysteine, or 3-mercaptoproline
XXXXXXXXXXXXXXX

SEQ ID NO: 724 - protein, 15 aa, Artificial Sequence
  Description of Artificial Sequence: Synthetic peptide
SHTCEICAFAACAGC

SEQ ID NO: 725 - protein, 15 aa, Artificial Sequence
  Description of Artificial Sequence: Synthetic peptide
PGTCEICAYAACTGC

SEQ ID NO: 726 - protein, 15 aa, Artificial Sequence
  Description of Artificial Sequence: Synthetic peptide
SHTCEICANAACAGC

SEQ ID NO: 727 - protein, 15 aa, Artificial Sequence
  Description of Artificial Sequence: Synthetic peptide
SHTCELCANAACAGC

SEQ ID NO: 728 - protein, 15 aa, Artificial Sequence
  Description of Artificial Sequence: Synthetic peptide
SHTCEVCANAACAGC

SEQ ID NO: 729 - protein, 15 aa, Artificial Sequence
  Description of Artificial Sequence: Synthetic peptide
SHTCEYCANAACAGC

SEQ ID NO: 730 - protein, 15 aa, Artificial Sequence
  Description of Artificial Sequence: Synthetic peptide
SHTCEICANAACAGC

SEQ ID NO: 731 - protein, 15 aa, Artificial Sequence
  Description of Artificial Sequence: Synthetic peptide
SHTCELCANAACAGC

SEQ ID NO: 732 - protein, 15 aa, Artificial Sequence
  Description of Artificial Sequence: Synthetic peptide
SHTCEVCANAACAGC

SEQ ID NO: 733 - protein, 15 aa, Artificial Sequence
  Description of Artificial Sequence: Synthetic peptide
SHTCEYCANAACAGC

SEQ ID NO: 734 - protein, 15 aa, Artificial Sequence
  Description of Artificial Sequence: Synthetic peptide
SHTCEICANAACAGC

SEQ ID NO: 735 - protein, 15 aa, Artificial Sequence
  Description of Artificial Sequence: Synthetic peptide
SHTCELCANAACAGC

SEQ ID NO: 736 - protein, 15 aa, Artificial Sequence
  Description of Artificial Sequence: Synthetic peptide
SHTCEVCANAACAGC

SEQ ID NO: 737 - protein, 15 aa, Artificial Sequence
  Description of Artificial Sequence: Synthetic peptide
SHTCEYCANAACAGC

SEQ ID NO: 738 - protein, 15 aa, Artificial Sequence
  Description of Artificial Sequence: Synthetic peptide
SHTCEICANAACAGC

SEQ ID NO: 739 - protein, 15 aa, Artificial Sequence
  Description of Artificial Sequence: Synthetic peptide
SHTCELCANAACAGC

SEQ ID NO: 740 - protein, 15 aa, Artificial Sequence
  Description of Artificial Sequence: Synthetic peptide
SHTCEVCANAACAGC

SEQ ID NO: 741 - protein, 15 aa, Artificial Sequence
  Description of Artificial Sequence: Synthetic peptide
SHTCEYCANAACAGC

SEQ ID NO: 742 - protein, 15 aa, Artificial Sequence
  Description of Artificial Sequence: Synthetic peptide
NDECEICANAACAGC

SEQ ID NO: 743 - protein, 15 aa, Artificial Sequence
  Description of Artificial Sequence: Synthetic peptide
NDECELCANAACAGC

SEQ ID NO: 744 - protein, 15 aa, Artificial Sequence
  Description of Artificial Sequence: Synthetic peptide
NDECEVCANAACAGC

SEQ ID NO: 745 - protein, 15 aa, Artificial Sequence
  Description of Artificial Sequence: Synthetic peptide
NDECEYCANAACAGC

SEQ ID NO: 746 - protein, 15 aa, Artificial Sequence
  Description of Artificial Sequence: Synthetic peptide
NDECEICANAACAGC

SEQ ID NO: 747 - protein, 15 aa, Artificial Sequence
  Description of Artificial Sequence: Synthetic peptide
NDECELCANAACAGC

SEQ ID NO: 748 - protein, 15 aa, Artificial Sequence
  Description of Artificial Sequence: Synthetic peptide
NDECEVCANAACAGC

SEQ ID NO: 749 - protein, 15 aa, Artificial Sequence
  Description of Artificial Sequence: Synthetic peptide
NDECEYCANAACAGC

SEQ ID NO: 750 - protein, 15 aa, Artificial Sequence
  Description of Artificial Sequence: Synthetic peptide
NDECEICANAACAGC

SEQ ID NO: 751 - protein, 15 aa, Artificial Sequence
  Description of Artificial Sequence: Synthetic peptide
NDECELCANAACAGC

SEQ ID NO: 752 - protein, 15 aa, Artificial Sequence
  Description of Artificial Sequence: Synthetic peptide
NDECEVCANAACAGC

SEQ ID NO: 753 - protein, 15 aa, Artificial Sequence
  Description of Artificial Sequence: Synthetic peptide
NDECEYCANAACAGC

SEQ ID NO: 754 - protein, 15 aa, Artificial Sequence
  Description of Artificial Sequence: Synthetic peptide
NDECEICANAACAGC

SEQ ID NO: 755 - protein, 15 aa, Artificial Sequence
  Description of Artificial Sequence: Synthetic peptide
NDECELCANAACAGC

SEQ ID NO: 756 - protein, 15 aa, Artificial Sequence
  Description of Artificial Sequence: Synthetic peptide
NDECEVCANAACAGC

SEQ ID NO: 757 - protein, 15 aa, Artificial Sequence
  Description of Artificial Sequence: Synthetic peptide
NDECEYCANAACAGC

SEQ ID NO: 758 - protein, 22 aa, Artificial Sequence
  Description of Artificial Sequence: Synthetic peptide
NSSNSSNYCCEKCCNPACTGCY

SEQ ID NO: 759 - protein, 16 aa, Artificial Sequence
  Description of Artificial Sequence: Synthetic peptide
NFCCETCCNPACTGCY

SEQ ID NO: 760 - protein, 16 aa, Artificial Sequence
  Description of Artificial Sequence: Synthetic peptide
NFCCETCCNPACTGCY

SEQ ID NO: 761 - protein, 16 aa, Artificial Sequence
  Description of Artificial Sequence: Synthetic peptide
NFCCETCCNPACTGCY

SEQ ID NO: 762 - protein, 16 aa, Artificial Sequence
  Description of Artificial Sequence: Synthetic peptide
NFCCEYCCNPACTGCY

SEQ ID NO: 763 - protein, 16 aa, Artificial Sequence
  Description of Artificial Sequence: Synthetic peptide
  MOD_RES (1)..(1): D-amino acid
  MOD_RES (16)..(16): D-amino acid
NFCCEYCCNPACTGCY

SEQ ID NO: 764 - protein, 16 aa, Artificial Sequence
  Description of Artificial Sequence: Synthetic peptide
  MOD_RES (16)..(16): D-amino acid
NFCCEYCCNPACTGCY

SEQ ID NO: 765 - protein, 16 aa, Artificial Sequence
  Description of Artificial Sequence: Synthetic peptide
  MOD_RES (1)..(1): D-amino acid
NFCCEYCCNPACTGCY

SEQ ID NO: 766 - protein, 16 aa, Artificial Sequence
  Description of Artificial Sequence: Synthetic peptide
DEECELCVNVACTGCL

SEQ ID NO: 767 - protein, 15 aa, Artificial Sequence
  Description of Artificial Sequence: Synthetic peptide
  MOD_RES (1)..(1): Any natural or unnatural amino acid or amino acid analogue
  MOD_RES (2)..(2): Any natural or unnatural amino acid or amino acid analogue
  MOD_RES (3)..(3): Any natural or unnatural amino acid or amino acid analogue
  MOD_RES (4)..(4): Cys, Pen, homocysteine, or 3-mercaptoproline
  MOD_RES (5)..(5): Any natural or unnatural amino acid or amino acid analogue
  MOD_RES (6)..(6): Any natural or unnatural amino acid or amino acid analogue
  MOD_RES (7)..(7): Cys, Pen, homocysteine, or 3-mercaptoproline
  MOD_RES (8)..(8): Any natural or unnatural amino acid or amino acid analogue
  MOD_RES (9)..(9): Any natural or unnatural amino acid or amino acid analogue
  MOD_RES (10)..(10): Any natural or unnatural amino acid or amino acid analogue
  MOD_RES (11)..(11): Any natural or unnatural amino acid or amino acid analogue
  MOD_RES (12)..(12): Cys, Pen, homocysteine, or 3-mercaptoproline
  MOD_RES (13)..(13): Any natural or unnatural amino acid or amino acid analogue
  MOD_RES (14)..(14): Any natural or unnatural amino acid or amino acid analogue
  MOD_RES (15)..(15): Any natural or unnatural amino acid or amino acid analogue
XXXXXXXXXXXXXXX

SEQ ID NO: 768 - protein, 15 aa, Artificial Sequence
  Description of Artificial Sequence: Synthetic peptide
QEECELCINMACTGY

SEQ ID NO: 769 - protein, 15 aa, Artificial Sequence
  Description of Artificial Sequence: Synthetic peptide
QEECETCINMACTGY

SEQ ID NO: 770 - protein, 15 aa, Artificial Sequence
  Description of Artificial Sequence: Synthetic peptide
QDECETCINMACTGY

SEQ ID NO: 771 - protein, 15 aa, Artificial Sequence
  Description of Artificial Sequence: Synthetic peptide
QDDCETCINMACTGY

SEQ ID NO: 772 - protein, 15 aa, Artificial Sequence
  Description of Artificial Sequence: Synthetic peptide
QEDCETCINMACTGY

SEQ ID NO: 773 - protein, 15 aa, Artificial Sequence
  Description of Artificial Sequence: Synthetic peptide
QEECEECINMACTGY

SEQ ID NO: 774 - protein, 15 aa, Artificial Sequence
  Description of Artificial Sequence: Synthetic peptide
QDECEECINMACTGY

SEQ ID NO: 775 - protein, 15 aa, Artificial Sequence
  Description of Artificial Sequence: Synthetic peptide
QDDCEECINMACTGY

SEQ ID NO: 776 - protein, 15 aa, Artificial Sequence
  Description of Artificial Sequence: Synthetic peptide
QEDCEECINMACTGY

SEQ ID NO: 777 - protein, 15 aa, Artificial Sequence
  Description of Artificial Sequence: Synthetic peptide
QEECEYCINMACTGY

SEQ ID NO: 778 - protein, 15 aa, Artificial Sequence
  Description of Artificial Sequence: Synthetic peptide
QDECEYCINMACTGY

SEQ ID NO: 779 - protein, 15 aa, Artificial Sequence
  Description of Artificial Sequence: Synthetic peptide
QDDCEYCINMACTGY

SEQ ID NO: 780 - protein, 15 aa, Artificial Sequence
  Description of Artificial Sequence: Synthetic peptide
QEDCEYCINMACTGY

SEQ ID NO: 781 - protein, 15 aa, Artificial Sequence
  Description of Artificial Sequence: Synthetic peptide
QEECEICINMACTGY

SEQ ID NO: 782 - protein, 15 aa, Artificial Sequence
  Description of Artificial Sequence: Synthetic peptide
QDECEICINMACTGY

SEQ ID NO: 783 - protein, 15 aa, Artificial Sequence
  Description of Artificial Sequence: Synthetic peptide
QDDCEICINMACTGY

SEQ ID NO: 784 - protein, 15 aa, Artificial Sequence
  Description of Artificial Sequence: Synthetic peptide
QEDCEICINMACTGY

SEQ ID NO: 785 - protein, 16 aa, Artificial Sequence
  Description of Artificial Sequence: Synthetic peptide
QEECETCINMACTGCS

SEQ ID NO: 786 - protein, 16 aa, Artificial Sequence
  Description of Artificial Sequence: Synthetic peptide
QDECETCINMACTGCS

SEQ ID NO: 787 - protein, 16 aa, Artificial Sequence
  Description of Artificial Sequence: Synthetic peptide
QDDCETCINMACTGCS

SEQ ID NO: 788 - protein, 16 aa, Artificial Sequence
  Description of Artificial Sequence: Synthetic peptide
QEDCETCINMACTGCS

SEQ ID NO: 789 - protein, 16 aa, Artificial Sequence
  Description of Artificial Sequence: Synthetic peptide
QEECEECINMACTGCS

SEQ ID NO: 790 - protein, 16 aa, Artificial Sequence
  Description of Artificial Sequence: Synthetic peptide
QDECEECINMACTGCS

SEQ ID NO: 791 - protein, 16 aa, Artificial Sequence
  Description of Artificial Sequence: Synthetic peptide
QDDCEECINMACTGCS

SEQ ID NO: 792 - protein, 16 aa, Artificial Sequence
  Description of Artificial Sequence: Synthetic peptide
QEDCEECINMACTGCS

SEQ ID NO: 793 - protein, 16 aa, Artificial Sequence
  Description of Artificial Sequence: Synthetic peptide
QEECEYCINMACTGCS

SEQ ID NO: 794 - protein, 16 aa, Artificial Sequence
  Description of Artificial Sequence: Synthetic peptide
QDECEYCINMACTGCS

SEQ ID NO: 795 - protein, 16 aa, Artificial Sequence
  Description of Artificial Sequence: Synthetic peptide
QDDCEYCINMACTGCS

SEQ ID NO: 796 - protein, 16 aa, Artificial Sequence
  Description of Artificial Sequence: Synthetic peptide
QEDCEYCINMACTGCS

SEQ ID NO: 797 - protein, 16 aa, Artificial Sequence
  Description of Artificial Sequence: Synthetic peptide
QEECEICINMACTGCS

SEQ ID NO: 798 - protein, 16 aa, Artificial Sequence
  Description of Artificial Sequence: Synthetic peptide
QDECEICINMACTGCS

SEQ ID NO: 799 - protein, 16 aa, Artificial Sequence
  Description of Artificial Sequence: Synthetic peptide
QDDCEICINMACTGCS

SEQ ID NO: 800 - protein, 16 aa, Artificial Sequence
  Description of Artificial Sequence: Synthetic peptide
QEDCEICINMACTGCS

SEQ ID NO: 801 - protein, 4 aa, Unknown
  Description of Unknown: DEAD motif
DEAD

SEQ ID NO: 802 - protein, 4 aa, Unknown
  Description of Unknown: KDEL motif
KDEL

(19) United States
(12) Patent Application Publication
(10) Pub. No.: US 2023/0279491 A1
(43) Pub. Date: Sep. 7, 2023

(54) TREATMENTS FOR A SUB-POPULATION OF INFLAMMATORY BOWEL DISEASE PATIENTS

(71) Applicant: CEDARS-SINAI MEDICAL CENTER, Los Angeles, CA (US)

(72) Inventors: Rebecca GONSKY, Los Angeles, CA (US); Stephan R. TARGAN, Santa Monica, CA (US)

(21) Appl. No.: 18/008,146

(22) PCT Filed: Jun. 1, 2021
(86) PCT No.: PCT/US2021/035217

§ 371 Date: Dec. 2, 2022

Related U.S. Application Data
(60) Provisional application No. 63/034,308, filed on Jun. 3, 2020.
(60) Provisional application No. 63/044,202, filed on Jun. 25, 2020.
(60) Provisional application No. 63/164,401, filed on Mar. 22, 2021.

(51) Int. Cl. C12Q 1/6883

(52) U.S. Cl. CPC C12Q 1/6883; C12Q 2600/158; C12Q 2600/112; C12Q 2600/178

(57) ABSTRACT

Described herein are methods and systems for identifying subpopulations of patients having Crohn's disease, including populations at risk of developing stricturing or other severe disease, and populations susceptible to success or failure with surgical intervention. Further provided are therapies useful for treating subpopulations of patients having Crohn's disease.

### PRIORITY

This application claims the benefit of U.S. Provisional Patent Application Ser. No. 63/034,308 filed Jun. 3, 2020, U.S. Provisional Patent Application Ser. No. 63/044,202 filed Jun. 25, 2020, and U.S. Provisional Patent Application Ser. No. 63/164,401 filed Mar. 22, 2021, each of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on May 24, 2021, is named 56884-771_601_SL.txt and is 279,136 bytes in size.

### BACKGROUND

Inflammatory bowel disease (IBD) is a pathobiologically heterogeneous disease that includes Crohn's disease and ulcerative colitis. Defining distinct disease populations is critical for improved prognostic accuracy, targeted therapeutics and biomarker discovery.

### SUMMARY

Crohn's disease (CD) is a clinically heterogeneous disease characterized by chronic transmural inflammation. A key contributing factor to persistent inflammation is failure of treatment options to effectively initiate and sustain long term remission. The efficacy of the current therapeutic approaches to control inflammation through the use of immunosuppressive drugs or biological therapies is variable. Anti-TNF therapy failure is common with many patients exhibiting primary non-response, and a significant number of patients develop secondary failure unrelated to anti-drug antibody formation. In addition, more than 30% of patients acquire cumulative complications such as stricturing, penetrating and/or fistula phenotypes within 10 years of diagnosis. Thus, patients whose disease is refractory to therapeutic modulation or exhibiting complications often require surgical intervention for disease management.

Predicting severity of disease course at time of diagnosis and response to therapy are challenges faced by clinicians. The profound genetic and patho-biologic heterogeneity in IBD makes defining distinct disease populations difficult, but critical, as the success in drug development in unselected patient populations has been limited in scope or has failed. Thus, novel approaches are needed not only in developing better prognostic biomarkers but more importantly to identify distinct patient sub-populations likely to benefit the most from the development of new and more effective treatments halting the progressive course of disease.

Recent efforts have focused on developing CD biomarkers that can predict disease course and patient outcomes. Expression signatures and genetic associations have added to our understanding however, they only explain a small proportion of overall disease variance. Moreover, the vast majority of these studies has focused on identifying factors driving disease progression when comparing CD patient to control subjects or patients with mild disease or naive to treatment to those with severe disease. Gene expression studies focusing on the patient population with refractory disease who fail therapeutic intervention with resistant complicated disease necessitating surgical intervention have been rare. Yet, understanding of the underlying pathobiology involved in this medically needy CD patient population, with a more severe clinical disease phenotype has the potential for the development of patient subtype targeted therapeutics that will enhance treatment efficacy.

In one aspect, provided herein are gene expression profiles within matched mucosal and circulating T cells obtained from CD patients with refractory disease at the time of surgery for disease management. In some embodiments, severe CD can be stratified into two distinct subtypes based on peripheral T cell gene expression. Circulating T cells, from what is classified as CD-PBmu subtype compared to CD-PBT, exhibit a mucosal-like transcriptomic signature and altered T cell subset composition that is associated with clinical features of complicated disease. A defining hallmark for CD-PBmu subtype is marked downregulation of pro-inflammatory cytokine, chemokine and adhesion molecule expression following surgery. In one aspect, therapeutics are selected for treating a severe CD patient population, such as a PB-mu subtype. In some embodiments, the PB-mu subtype is associated with perianal disease/fistula, stricturing disease, recurrence, or increased immune reactivity to a microbial antigen, or a combination thereof.

In one aspect, provided herein is a method of determining a Crohn's Disease (CD) subtype status in a subject having CD, wherein the status comprises distinguishing a CD PBmucosal (CD-PBmu) subtype from a non-CD-PBmu subtype, the method comprising: detecting expression of one or more genes from Tables 1A-1B in a biological sample from the subject to obtain an expression profile comprising the expression levels of each of the one or more genes in the biological sample, and determining the CD subtype status of the subject based upon the expression profile, wherein an increased level of expression in the one or more genes in the biological sample as compared to a reference expression profile indicates status of CD-PBmu subtype as distinguished from a non-CD-PBmu subtype.

In one aspect, provided herein is a method of selecting a treatment for a subject having a Crohn's Disease (CD) PBmucosal (CD-PBmu) subtype, the method comprising: (a) determining a level of expression of one or more genes from Tables 1A-1B in a biological sample obtained from the subject having CD; (b) detecting an expression profile comprising an increase in the level of expression of the one or more genes in the biological sample, relative to a reference expression profile; and (c) identifying the subject as a candidate for treatment of Crohn's Disease based upon the expression profile that is detected in (b). The method of claim 1 or claim 2, wherein the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.

In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPINGL SOD3, SYK, TBC1D3, TBC1D9, TPSB2, MIR155HG, or UBD, or a combination thereof. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, determining a level of expression of one or more genes comprises utilizing an assay selected from the group consisting of an RNA sequencing method, a microarray method, and quantitative polymerase chain reaction (qPCR). In some embodiments, determining a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the CD is associated with perianal disease/fistula. In some embodiments, the CD is associated with stricturing disease. In some embodiments, the CD is associated with recurrence. In some embodiments, the CD is associated with increased immune reactivity to a microbial antigen. In some embodiments, the expression of at least one of the one or more genes in the biological sample is at least 2-fold greater than in the reference expression profile. In some embodiments, the reference expression profile comprises expression levels of the one or more genes of one or more subjects who do not have IBD or have a PBT subtype of CD. In some embodiments, the reference expression profile is stored in a database. In some embodiments, the method further comprises treating the subject with a therapeutic agent.

Further provided is a method of treating a subject having a Crohn's Disease (CD) PBmucosal (CD-PBmu) subtype, the method comprising: (a) determining a level of expression of one or more genes from Tables 1A-1B in a biological sample obtained from the subject having CD; (b) detecting an expression profile comprising an increase in the level of expression of the one or more genes in the biological sample, relative to a reference expression profile; and (c) administering to the subject a therapeutic agent against Crohn's Disease based upon the expression profile that is detected in (b).

In some embodiments, the therapeutic agent comprises a therapeutic of Table 20B; a protein, peptide, nucleic acid, or compound that targets a molecule of Tables 14, 15, 17A-17B, or 20A; or a compound that targets a molecule in a pathway of one or more genes of Table 17B; or any combination thereof. In some embodiments, the therapeutic agent comprises a modulator of miR-155. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen.

In some embodiments, the biological sample comprises a blood sample or is purified from a blood sample of the subject. In some embodiments, the subject is not responsive to anti-TNFα therapy. In some embodiments, the subject has or is susceptible to having stricturing disease. In some embodiments, the subject has or is susceptible to having increased length of bowel resection.

Further provided is a method for processing or analyzing a biological sample from a subject, comprising: (a) obtaining the biological sample comprising gene expression products, wherein the subject has or is suspected of having Crohn's Disease (CD); (b) subjecting the biological sample to an assay by sequencing, array hybridization, and/or nucleic acid amplification to yield a data set including data corresponding to gene expression product levels; (c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and (d) electronically outputting a report that identifies the classification of the biological sample as positive or negative for the CD subtype.

In some embodiments, the sample is classified at an accuracy of at least about 90%, 91%, 92%, 93%, 94%, 95%, 96%, 97%, 98%, or 99%. In some embodiments, the gene expression product comprises ribonucleic acid. In some embodiments, the trained algorithm is trained with one or more datasets of gene expression product levels obtained from the plurality of training samples. In some embodiments, the gene expression products comprise one or more genes from Tables 1A-1B.

In some embodiments, the method further comprises administering to the subject a kinase inhibitor. In some embodiments, the method further comprises administering to the subject a modulator of a molecule of Table 14. In some embodiments, the method further comprises administering to the subject a modulator of a molecule of Table 15. In some embodiments, the method further comprises administering to the subject a modulator of a molecule of Table 17A. In some embodiments, the method further comprises administering to the subject a modulator of a molecule of Table 17B. In some embodiments, the method further comprises administering to the subject a modulator of a molecule of Table 20A. In some embodiments, the method further comprises administering to the subject a modulator of a compound that targets a molecule in a pathway of one or more genes of Table 17B. In some embodiments, the method further comprises administering to the subject a therapeutic of Table 20B. In some embodiments, the method further comprises administering to the subject a an anti-TL1A antibody. In some embodiments, the anti-TL1A antibody comprises CDRs comprising SEQ ID NOS: 346-351.

Further provided is a panel of biomarker nucleic acids comprising at least 10 but less than 100 contiguous nucleobases of a plurality of genes, the plurality of genes comprising two or more genes from Tables 1A-1B.

Further aspects disclosed herein provide a method of determining a Crohn's Disease (CD) subtype status in a subject having CD, wherein the status comprises distinguishing a CD PBmucosal (CD-PBmu) subtype from a non-CD-PBmu subtype, the method comprising: detecting expression of one or more genes from Tables 1A-1B in a biological sample from the subject to obtain an expression profile comprising the expression levels of each of the one or more genes in the biological sample, and determining the CD subtype status of the subject based upon the expression profile, wherein an increased level of expression in the one or more genes as compared to a reference expression profile indicates status of CD-PBmu subtype as distinguished from a non-CD-PBmu subtype. In some embodiments, the one or more genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 genes. In some embodiments, the one or more genes comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or all of the genes in Tables 1A-1B. In some embodiments, the one or more genes comprises ADH4. In some embodiments, the one or more genes comprises ALG1L. In some embodiments, the one or more genes comprises BCDIN3D. In some embodiments, the one or more genes comprises C1orf106. In some embodiments, the one or more genes comprises C2. In some embodiments, the one or more genes comprises CCDC144NL. In some embodiments, the one or more genes comprises CEACAM5. In some embodiments, the one or more genes comprises CTAGE8. In some embodiments, the one or more genes comprises DDX11L2. In some embodiments, the one or more genes comprises DPPA4. In some embodiments, the one or more genes comprises DUSP19. In some embodiments, the one or more genes comprises FGB. In some embodiments, the one or more genes comprises GP2. In some embodiments, the one or more genes comprises GYPE. In some embodiments, the one or more genes comprises HSD3B7. In some embodiments, the one or more genes comprises HUNK. In some embodiments, the one or more genes comprises JAM2. In some embodiments, the one or more genes comprises KCNE3. In some embodiments, the one or more genes comprises KRT42P. In some embodiments, the one or more genes comprises LYZ. In some embodiments, the one or more genes comprises MLLT10P1. In some embodiments, the one or more genes comprises NAP1L6. In some embodiments, the one or more genes comprises NEURL3. In some embodiments, the one or more genes comprises NPIPB9. In some embodiments, the one or more genes comprises PANK1. In some embodiments, the one or more genes comprises PKIB. In some embodiments, the one or more genes comprises RHOU. In some embodiments, the one or more genes comprises RPSAP9. In some embodiments, the one or more genes comprises SHCBP1. In some embodiments, the one or more genes comprises SIGLEC8. In some embodiments, the one or more genes comprises SLC15A2. In some embodiments, the one or more genes comprises SLC25A34. In some embodiments, the one or more genes comprises SLC6A20. In some embodiments, the one or more genes comprises SLC9B1. In some embodiments, the one or more genes comprises SYNPO2L. In some embodiments, the one or more genes comprises TDGF1. In some embodiments, the one or more genes comprises ZNF491. In some embodiments, the one or more genes comprises ZNF620. In some embodiments, the one or more genes comprises ZNF69. In some embodiments, the one or more genes comprises CXCL16. In some embodiments, the one or more genes comprises CD68. In some embodiments, the one or more genes comprises CD300E. In some embodiments, the expression of at least one of the one or more genes in the biological sample is at least 2-fold greater than in the reference expression profile. In some embodiments, the reference expression profile comprises expression levels of the one or more genes of one or more subjects who do not have IBD or have a PBT subtype of CD. In some embodiments, detecting expression of the one or more genes comprises a RNA sequencing method. In some embodiments, detecting expression of the one or more genes comprises a microarray method. In some embodiments, detecting expression of the one or more genes comprises hybridization of a nucleic acid primer and/or probe to the biological sample, wherein the nucleic acid primer and/or probe comprises at least about 10 contiguous nucleobases of one of the one or more genes from Tables 1A-1B. In some embodiments, the reference expression profile is stored in a database. In some embodiments, the method further comprises treating the subject with a therapeutic agent. In some embodiments, the therapeutic agent comprises a therapeutic of Table 20B; a protein, peptide, nucleic acid, or compound that targets a molecule of Tables 14, 15, 17A-17B, 20A; or a compound that targets a molecule in a pathway of one or more genes of Table 17B; or any combination thereof. In some embodiments, the biological sample comprises a blood sample or is purified from a blood sample of the subject. In some embodiments, the subject is less than 18 years of age. In some embodiments, the subject is 18 years of age or older. In some embodiments, the subject is not responsive to anti-TNFα therapy. In some embodiments, the subject has or is susceptible to having stricturing disease. In some embodiments, the subject has or is susceptible to having increased length of bowel resection. In some embodiments, the method further comprises administering to the subject a modulator of a modulator of a molecule of Table 14. In some embodiments, the method further comprises administering to the subject a modulator of a molecule of Table 15. In some embodiments, the method further comprises administering to the subject a modulator of a molecule of Table 17A. In some embodiments, the method further comprises administering to the subject a modulator of a molecule of Table 17B. In some embodiments, the method further comprises administering to the subject a modulator of a molecule of Table 20A. In some embodiments, the method further comprises administering to the subject a modulator of a compound that targets a molecule in a pathway of one or more genes of Table 17B. In some embodiments, the method further comprises administering to the subject a therapeutic of Table 20B. In some embodiments, the method further comprises administering to the subject an anti-TL1A antibody. In some embodiments, the anti-TL1A antibody comprises CDRs comprising SEQ ID NOS: 346-351.

Further provided is a method comprising administering to the subject a modulator of a molecule of Table 14, wherein the subject is determined to have a CD-PBmu subtype as described in a method herein.

Further provided is a method comprising administering to the subject a modulator of a molecule of Table 15, wherein the subject is determined to have a CD-PBmu subtype as described in a method herein.

Further provided is a method comprising administering to the subject a modulator of a molecule of Table 17A, wherein the subject is determined to have a CD-PBmu subtype as described in a method herein.

Further provided is a method comprising administering to the subject a modulator of a molecule of Table 17B, wherein the subject is determined to have a CD-PBmu subtype as described in a method herein.

Further provided is a method comprising administering to the subject a modulator of a molecule of Table 20A, wherein the subject is determined to have a CD-PBmu subtype as described in a method herein.

Further provided is a method comprising administering to the subject a modulator of a compound that targets a molecule in a pathway of one or more genes of Table 17B, wherein the subject is determined to have a CD-PBmu subtype as described in a method herein.

Further provided is a method comprising administering to the subject a therapeutic of Table 20B, wherein the subject is determined to have a CD-PBmu subtype as described in a method herein.

Further provided is a method comprising administering to the subject an anti-TL1A antibody, wherein the subject is determined to have a CD-PBmu subtype as described in a method herein. In some embodiments, the anti-TL1A antibody comprises CDRs comprising SEQ ID NOS: 346-351.

Further aspects provide a method comprising treating a subject with a therapeutic agent that targets a molecule in a pathway of one or more genes selected from Tables 1A-1B, wherein the subject is determined to have a CD-PBmu subtype as described in a method herein. In some embodiments, the therapeutic agent comprises a peptide, nucleic acid, compound, or a combination thereof.

Further aspects provide a method comprising determining an increase or decrease in expression of a gene effectuated by a therapeutic agent in a subject, the method comprising detecting expression of the gene after administration of the therapeutic agent to the subject, wherein the gene is selected from Tables 1A-1B. In some embodiments, the therapeutic agent comprises a therapeutic of Table 20B; a protein, peptide, nucleic acid, or compound that targets a molecule of Tables 14, 15, 17A-17B, 20A; or a compound that targets a molecule in a pathway of one or more genes of Table 17B; or any combination thereof. In some embodiments, the expression is detected using a method described herein.

Further aspects provide a method comprising administering to the subject a kinase inhibitor, wherein the subject is determined to have a CD-PBmu subtype as described in a method herein. In some embodiments, the method further comprises administering to the subject a kinase inhibitor. In some embodiments, the kinase target of the kinase inhibitor is a kinase described herein. In some embodiments, the kinase target of the kinase inhibitor comprises a kinase of FIG. 6. In some embodiments, the kinase target of the kinase inhibitor comprises a kinase of FIG. 7C. In some embodiments, the kinase target of the kinase inhibitor comprises a kinase of FIG. 7D.

Further aspects provide a method for processing or analyzing a biological sample from a subject, comprising: (a) obtaining the biological sample comprising gene expression products, wherein the subject has or is suspected of having Crohn's Disease (CD); (b) subjecting the biological sample to an assay by sequencing, array hybridization, and/or nucleic acid amplification to yield a data set including data corresponding to gene expression product levels; (c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and (d) electronically outputting a report that identifies the classification of the biological sample as positive or negative for the CD subtype. In some embodiments, the sample is classified at an accuracy of at least about 90%, 91%, 92%, 93%, 94%, 95%, 96%, 97%, 98%, or 99%. In some embodiments, the gene expression product comprises ribonucleic acid. In some embodiments, the assay comprises using one or more of the following: microarray, sequencing, SAGE, blotting, reverse transcription, and quantitative polymerase chain reaction (PCR). In some embodiments, the trained algorithm is trained with one or more datasets of gene expression product levels obtained from the plurality of training samples. In some embodiments, the gene expression products comprise one or more genes from Tables 1A-1B.

Further aspects provide a composition comprising at least 10 but less than 100 contiguous nucleobases of a gene of Tables 1A-1B or its complement, and a detectable label.

Further aspects provide a panel of biomarker nucleic acids comprising at least 10 but less than 100 contiguous nucleobases of a plurality of genes, the plurality of genes comprising two or more genes from Tables 1A-1B.

Further aspects provide a composition comprising an agent that modulates expression and/or activity of a molecule in a pathway of one or more genes selected from Tables 1A-1B.

Further aspects disclosed herein provide a method for selecting a treatment for a subject having or suspected of having Crohn's Disease, comprising: (a) obtaining a biological sample comprising gene expression products from the subject; (b) subjecting the biological sample to an assay to yield a data set including data corresponding to gene expression product levels; (c) in a programmed computer, inputting said data including said gene expression product levels from (b) to a trained algorithm to generate a classification of said sample as positive for a CD-PBmu subtype based on detection of an expression profile comprising an increase in the gene expression levels compared to a reference expression profile, wherein the trained algorithm is trained with a plurality of training samples, and wherein said biological sample is independent of said plurality of training samples; (d) electronically outputting a report that identifies the classification of the biological sample as positive for the CD-PBmu subtype; and (e) correlating the positive CD-PBmu subtype with a treatment comprising administration of a modulator of miR-155. In some embodiments, the gene expression products comprise RNA. In some embodiments, the assay comprises using one or more of a microarray, sequencing, and qPCR. In some embodiments, the trained algorithm is trained with one or more datasets of gene expression product levels obtained from the plurality of training samples. In some embodiments, the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof. In some embodiments, the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPINGL SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof. In some embodiments, the increase in the gene expression product levels is at least 2-fold greater than in the reference expression profile. In some embodiments, the reference expression profile comprises expression levels of the one or more genes of one or more subjects that do not have CD. In some embodiments, the biological sample comprises a blood sample or is purified from a blood sample of the subject. In some embodiments, the method comprises treating the subject by administering to the subject the miR-155 modulator. In some embodiments, the method comprises optimizing a therapeutic regimen of the subject comprising increasing or decreasing a dosage amount of the miR-155 modulator. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen. In some embodiments, expression of miR-155 is elevated in the sample from the subject as compared to a reference expression profile of one or more subjects who do not comprise the CD PBmu subtype. In some embodiments, the method comprises treating the subject with the miR-155 modulator.

In another aspect, provided herein is a method for selecting a treatment for a subject having or suspected of having Crohn's Disease, comprising: (a) obtaining a biological sample comprising MIR155 from the subject; (b) subjecting the biological sample to an assay to yield a data set including data corresponding to expression level of the MIR155; (c) in a programmed computer, inputting said data including said expression level of the MIR155 from (b) to a trained algorithm to generate a classification of said sample as positive for a subtype based on detection of an expression profile comprising an increase in the expression level of MIR155 compared to a reference expression profile, wherein the trained algorithm is trained with a plurality of training samples, and wherein said biological sample is independent of said plurality of training samples; (d) electronically outputting a report that identifies the classification of the biological sample as positive for the subtype; and (e) correlating the positive subtype with a treatment comprising administration of a modulator of miR-155. In some embodiments, the assay comprises using one or more of a microarray, sequencing, and qPCR. In some embodiments, the increase in the gene expression product levels is at least 2-fold greater than in the reference expression profile. In some embodiments, the reference expression profile comprises expression levels of MIR155 of one or more subjects that do not have CD. In some embodiments, the biological sample comprises a blood sample or is purified from a blood sample of the subject. In some embodiments, the method comprises treating the subject by administering to the subject the miR-155 modulator. In some embodiments, the method comprises optimizing a therapeutic regimen of the subject comprising increasing or decreasing a dosage amount of the miR-155 modulator. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen.

In another aspect, provided herein is a method of treating Crohn's disease (CD) in a subject, the method comprising administering to the subject a therapeutically effective amount of a miR-155 modulator, provided the subject is identified as having a CD-PBmu subtype by: (a) detecting an expression profile comprising an increase in a level of expression of one or more genes in a biological sample from the subject, relative to a reference expression profile; and (b) identifying the subject as having a CD-PBmu subtype based upon the expression profile that is detected in (b). In some embodiments, the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPINGL UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A, or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPINGL SOD3, SYK, TBC1D3, TBC1D9, TPSB2, MIR155HG, or UBD, or a combination thereof. In some embodiments, the one or more genes comprises at least 10 of the one or more genes. In some embodiments, the one or more genes comprises between about 10-27 of the one or more genes. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, detecting the expression profile comprises detecting the increase in the level of expression of the one or more genes by: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen. In some embodiments, expression of miR-155 is elevated in the sample from the subject as compared to a reference expression profile of one or more subjects who do not comprise the CD PBmu subtype. In some embodiments, the method comprises treating the subject with the miR-155 modulator.

In another aspect, provided herein is a method of treating Crohn's disease (CD) in a subject, the method comprising administering to the subject a therapeutically effective amount of a miR-155 modulator, provided the subject is identified as having a CD-PBmu subtype by: (a) detecting an expression profile comprising an increase in a level of expression of MIR155 in a biological sample from the subject, relative to a reference expression profile; and (b) identifying the subject as having a CD-PBmu subtype based upon the expression profile that is detected in (b). In some embodiments, the increase in the level of expression of MIR155 in the biological sample is at least 2-fold greater than in the reference expression profile. In some embodiments, the reference expression profile comprises expression levels of MIR155 of one or more subjects that do not have CD. In some embodiments, detecting the expression profile comprises detecting the increase in the level of expression of MIR155 by: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen. In some embodiments, the method comprises treating the subject with the miR-155 modulator.

In another aspect, provided herein is a method of selecting a treatment for a subject having Crohn's Disease (CD), the method comprising: (a) measuring a level of expression of one or more genes from Tables 1A-1B in a biological sample obtained from the subject having CD; (b) detecting an expression profile comprising an increase in the level of expression of the one or more genes in the biological sample, relative to a reference expression profile; and (c) identifying the subject as a candidate for treatment with a modulator of miR-155based upon the expression profile that is detected in (b). In some embodiments, the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPINGL UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPINGL SOD3, SYK, TBC1D3, TBC1D9, TPSB2, MIR155HG, or UBD, or a combination thereof. In some embodiments, the one or more genes comprises at least 10 of the one or more genes. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, measuring a level of expression of one or more genes comprises utilizing an assay selected from the group consisting of an RNA sequencing method, a microarray method, and quantitative polymerase chain reaction (qPCR). In some embodiments, measuring a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, the method comprises treating the subject by administering the modulator of miR-155 to the subject. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen. In some embodiments, the method comprises optimizing a therapeutic regimen of the subject comprising increasing or decreasing a dosage amount of the modulator of miR-155 administered to the subject for the treatment of the CD, based on the expression profile. In some embodiments, the biological sample comprises a blood sample or is purified from a blood sample of the subject.

In another aspect, provided herein is a method of determining a Crohn's Disease (CD) subtype in a subject having CD, the method comprising: (a) measuring a level of expression of MIR155 in a biological sample obtained from a subject having CD; (b) detecting an expression profile comprising an increase in the level of expression of MIR155 in the biological sample, relative to a reference expression profile; and (c) identifying the subject as having a CD-PBmu subtype based upon the expression profile that is detected in (b). In some embodiments, the increase in the level of expression of MIR155 in the biological sample is at least 2-fold greater than in the reference expression profile. In some embodiments, the reference expression profile comprises expression levels of MIR155 of one or more subjects that do not have CD. In some embodiments, measuring a level of expression comprises utilizing an assay selected from the group consisting of an RNA sequencing method, a microarray method, and quantitative polymerase chain reaction (qPCR). In some embodiments, measuring a level of expression of MIR155 comprises: (a) contacting the biological sample with a nucleic acid primer and/or detectable nucleic acid probe; and (b) hybridizing the nucleic acid primer and/or detectable nucleic acid probe to a nucleic acid sequence of MIR155, wherein the detectable nucleic acid probe comprises a nucleic acid sequence comprising at least about 10 contiguous nucleic acids of MIR155. In some embodiments, the method comprises treating the subject by administering a therapeutic agent to the subject. In some embodiments, the method comprises optimizing a therapeutic regimen of the subject comprising increasing or decreasing a dosage amount of a therapeutic agent administered to the subject for the treatment of the CD, based on the CD-PBmu subtype. In some embodiments, the therapeutic agent comprises a miR-155 modulator. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen. In some embodiments, the biological sample comprises a blood sample or is purified from a blood sample of the subject.

In another aspect, provided herein is a method of treating an inflammatory disease in a subject, the method comprising: administering to the subject a modulator of miR-155, provided that a sample comprising gene expression products from the subject comprises a PBmu subtype based on detection of an expression profile comprising an increase in gene expression level of one or more gene products compared to a reference expression profile of the one or more gene products. In some embodiments, the inflammatory disease comprises inflammatory bowel disease. In some embodiments, the inflammatory bowel disease comprises Crohn's disease. In some embodiments, the gene products comprise RNA. In some embodiments, the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof. In some embodiments, the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof. In some embodiments, the increase in the gene expression product levels is at least 2-fold greater than in the reference expression profile. In some embodiments, the reference expression profile comprises expression levels of the one or more genes of one or more subjects that do not have CD. In some embodiments, the biological sample comprises a blood sample or is purified from a blood sample of the subject. In some embodiments, the method comprises optimizing a therapeutic regimen of the subject comprising increasing or decreasing a dosage amount of the miR-155 modulator. In some embodiments, the miR-155 modulator comprises an inhibitor of miR-155. In some embodiments, the miR-155 modulator comprises one or more oligonucleotides of Tables 3-12. In some embodiments, the miR-155 modulator comprises Cobomarsen.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1A is a principal component analysis (PCA) of CD3+ T cell gene expression from the lamina propria or periphery isolated from CD or non-IBD individuals.

FIG. 1B is an unsupervised hierarchical clustering defining two CD peripheral expression CD-PBmu and CD-PBT subtypes.

FIG. 1C is a heat map of 1944 genes differentially expressed between PBmu and PBT subtypes (p value <0.001 and fold change >2).

FIG. 1D is a pathway analysis of PBmu differentially expressed genes.

FIG. 1E is a t-SNE plot of deconvoluted CD3+ immune cell enrichment scores.

FIG. 1F shows a heat map and p values of altered T cell subset abundance in CD-PBmu versus PBT subtypes (Mann-Whitney test).

FIG. 1G and FIG. 1H show that PB-mu expression signature can be applied to stratify CD patients who failed anti-TNF therapy. The 1944 genes defining the CD PBmu and PBT subtypes identified similar subtypes from expression data isolated from a CD cohort of patients who has failed anti-TNF therapy. FIG. 1G shows the principal component analysis and FIG. 1H shows hierarchical clustering of the 204 whole blood samples.

FIG. 1I is a heat mat of 1566 genes differentially expressed between Cd-PBmu and CD-PBT subtypes (p value<0.001, FDR<0.002, fold change >2). FIG. 1J is a heat map of 1566 CD-PBmu and CD-PBT differentially expressed genes across all LPT and PBT samples. FIG. 1K is a pathway analysis of CD-PBmu differentially expressed genes. FIG. 1L is a correlation matrix plot between the CD-PBmu NKT and CD4+/CD8+ T cell subset enrichment scores showing no significant positive or negative correlation between NKT and CD4+/CD8+ cell enrichment scores.

FIG. 1M and FIG. 1N show Gene Set Variation Analysis (GSVA) scores for the 1566 differentially expressed genes (DEG 1566) and 42 biomarker gene panel. FIG. 1M shows that CD-PBmu vs CD-PBT GSVA scores are elevated in CD-PBmu. FIG. 1N shows a positive correlation with NKT and negative correlation with T cell subset enrichment scores.

FIGS. 1O-1Q show CD-PBmu expression signature stratifies CD patients who failed on anti-TNF therapy. The genes defining the CD-PBmu vs CD-PBT subtypes (FIG. 1I) were used to identify similar subtypes from an independent CD cohort of patients who have failed anti-TNF therapy. FIG. 1O is a principal component analysis (PCA). FIG. 1P hierarchical clustering of CD whole blood expression data identifies two CD patient subtypes. FIG. 1Q is a heat map based on cellular enrichment scores using xCell bioinformatics tool. Enrichment of NKT and depletion of CD4+/CD8+ T cell subsets were associated with the samples classified as PBmu-like subtype.

FIG. 1R is a heat map based on cellular enrichment scores using xCell bioinformatics tool. Clusters were generated using a random gene probe set as input.

FIGS. 2A-2C show post-operative changes in PBmu gene expression profile. FIG. 2A is a heat map and FIG. 2B is a volcano plot of 877 genes differentially expressed in CD-PBmu subtype at time of surgery vs post-operatively (p value <0.001, FDR <0.01). FIG. 2C shows attenuation of pro-inflammatory cytokine, chemokine, and adhesion molecule expression in CD-PBmu subsequent to surgery. Bars on the left show p value and bars on the right show corresponding fold change.

FIGS. 2D-2E demonstrate that PBmu gene expression profile reverts to that of CD PBT following surgery. FIG. 2D is a hierarchical clustering and heatmap of the 1566 genes defining the CD-PBmu and PBT subtypes comparing peripheral CD3+ T cell expression in all samples prior to surgery and post-operatively. Asterix denotes samples that did not cluster as predicted. FIG. 2E are scatter plots showing high correlation of gene expression between PBmu subtype samples following surgery and PBT subtype pre- or post-surgery.

FIGS. 3A-3F demonstrates validation of CD-PBmu gene signature reversion following surgery in a cohort of subjects comparing samples isolated at time of surgery to post-operative samples from same individuals (n=19). FIG. 3A is a PCA and FIG. 3B is a hierarchical clustering of samples at time of surgery. FIG. 3C is a heatmap of expression data for the same genes defining the CD-PBmu and CD-PBT subtypes in FIGS. 1A-1F. FIG. 3D is a PCA analysis of samples at surgery and post-operatively for CD-PBmu. FIG. 3E is a PCA analysis of samples at surgery and post-operatively for CD-PBT. FIG. 3F is a heatmap of expression data from genes previously defined in CD-PBmu samples pre and post-surgery in FIG. 2A-2C (624/901 genes were differentially expressed, p value<0.05). No genes were differentially expressed in CD-PBT when comparing pre to post surgery.

FIG. 4A demonstrates a CD PBmu peripheral gene signature shows similar co-expression with ileal tissue. ARCHS4 generated t-SNE plots of gene signature from 100 differentially up-regulated genes in PBmu vs PBT overlaps with similar co-expression from ileal tissue. Purple corresponds to CD PBmu up-regulated genes. Blue corresponds to ileal tissue.

FIG. 4B A CD-PBmu peripheral gene signature shows similar co-expression with ileal/colonic tissue. ARCHS4 generated t-SNE plots of gene signature from differentially up-regulated gene panel in CD-PBmu versus CD-PBT overlaps with similar co-expression from ileal and colonic tissue. In the top panel, blue corresponds to ileal tissue, green corresponds to colon tissue. In the middle panel, purple corresponds to 193 differentially up-regulated genes. In the bottom panel, orange corresponds to the 42 biomarkers.

FIG. 4C is a table with the source of overlapping bowel tissue with similar co-expression to CD-PBmu and 42 biomarker gene signatures.

FIG. 5 shows pathways enriched in the CD-PBmu 44 biomarker signature.

FIG. 6 shows that PBmu 44 biomarker signature is associated with expression of kinases as provided.

FIGS. 7A-7B show that 44 Biomarker expression gene panel correlates to PB-mu enriched NKT and depleted CD4+ memory T cell subsets. FIG. 7A is a correlation plot of biomarker gene panel expression versus enrichment scores for NKT cell and CD4+ memory T cell subsets. FIG. 7B is a heatmap of correlation values of gene expression versus enrichment scores for the biomarker panel. Arrows highlight a reported TWAS IBD association. Below the heatmap is a bar plot showing the proportion of significant gene panel correlation with T cell subsets.

FIGS. 7C-7D show protein kinase signaling pathways identified correlating to expression of the CD-PBmu expression signature. FIG. 7C is a bar plot showing fold enhancement of kinase expression when comparing CD-PBmu versus CD-PBT prior to surgery (bars on the left) and selective decrease post-operatively for the PBmu subtype (bars on the right). The kinase signaling pathways include EEF2K, CAMK1D, ZAK, AK3, YES1, MELK, ADRBK2, MAP3K9, GK5, PANK1, MAP3K13, NEK8, ALPK1, SGK494, GNE, NEK5, ERBB3, PTK6, FLT1, TRPM6, DGKB, MOK, AXL, NEK2, and FGFR2. FIG. 7D is a bar plot showing upstream kinases that in some embodiments target PBmu differentially expressed gene putative substrates: PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1. The bars on the left show percent of targeted input gene set predicted as a substrate for individual kinases predicted using KEA3 analysis. Numbers at left represent mean rank. The bars on the right show corresponding p values for X2k kinase enrichment analysis for predicted upstream regulators. The arrows represent therapeutic kinase inhibitors currently in use or in clinical trials.

FIG. 7E shows expression of 42 biomarker gene panel correlates with CD-PBmu enriched NKT and depleted CD4+ memory T cell subsets. Heatmap of correlation values of gene expression versus enrichment scores for biomarker panel (right panel) and association with perianal penetrating disease and ASCA sero-positivity (left panel). FIG. 7F is a correlation plot of biomarker gene panel expression versus enrichment scores for NK T cell and CD4+ memory T cell subsets.

FIG. 8 shows clustering of CD monocytes to reveal two signatures: monocyte 1 subtype and monocyte 2 subtype.

FIG. 9 shows differential gene expression in monocyte 1 subtype versus monocyte 2 subtype.

FIG. 10A shows differentially expressed genes (DEG) in PBmu as compared to PBT in a genome wide association study (GWAS).

FIG. 10B shows enriched pathways that overlap with the GWAS DEG in CD-PBmu.

FIG. 11A shows expression of miR-155 is significantly increased in PB T-cells from patients with PBmu subtype when compared to both non-IBD and PBT subtype samples.

FIG. 11B shows expression of miR-155 is not significantly increased in LP T-cells from patients with LBmu subtype when compared to both non-IBD and LPT subtype samples.

FIG. 12 shows miR-155 expression is elevated in interferon gamma secreting CD4+ T-cells.

FIG. 13A shows treatment of T-cells to determine whether TL1A regulations miR-155 expression.

FIG. 13B shows TL1A mediated upregulation of miR-155.

FIG. 14 shows miR-155 mimic enhances interferon gamma and IL-22 secretion.

FIG. 15 shows miR-155 inhibition suppresses interferon gamma and IL-22 secretion.

FIG. 16 shows expression of TNFSF15 (the gene expressing TL1A) in patients having a PBmu subtype as compared to no expression in patients having the PBT subtype of CD.

FIGS. 17A-17F demonstrate that CD-PBmu altered T cell subset composition is associated with clinical and serological parameters of complicated disease. FIG. 17A demonstrates association of NKT enrichment with stricturing disease and perianal disease and CD4+/CD8+ T cell subset depletion in CD-PBmu with perianal disease/fistula, penetrating disease, stricturing disease and post-operative endoscopic recurrence (N=Rutgeerts score 0-1; Y=2-4). FIG. 17B demonstrates association of NKT enrichment and CD4+/CD8+ T cell subset depletion in CD-PBmu with ASCA seropositivity. FIG. 17 C demonstrates inverse correlation of serological quartile sum scores in CD-PBmu with of CD4+/CD8+ T cell subsets depletion. FIG. 17D demonstrates association of serological quartile sum scores in CD-PBmu with increased length of bowel resection. FIG. 17E and FIG. 17F show association of GSVA differential gene expression scores and NKT and CD4+ memory T cell scores with pre-op steroid use (FIG. 17E), stricturing disease (FIG. 17E) and ANCA sero-positivity (FIG. 17F) (blue circles correspond to CD-PBmu, red circles correspond to CD-PBT).

FIGS. 18A-18D show CD-PBT T cell subset composition is not associated with clinical and serological parameters of complicated disease. FIG. 18A demonstrates the association of NKT and CD4+/CD8+ T cell subset enrichment score with perianal disease/fistula, stricturing disease and post-operative endoscopic recurrence (N=Rutgeerts score 0-1; Y=2-4) FIG. 18B demonstrates no association of NKT or CD4+/CD8+ T cell subset enrichment score with ASCA seropositivity. FIG. 18C demonstrates no correlation of serological quartile sum scores with CD4/CD8 T cell subsets enrichment scores. FIG. 18D demonstrates no association of serological quartile sum scores in CD-PBmu with increased length of bowel resection.

### DETAILED DESCRIPTION

The present disclosure provides methods and systems for characterizing and treating patients having Crohn's disease (CD). In particular embodiments, a CD patient is characterized as having or not having a mucosal-like CD expression signature (CD-PBmu) by transcriptomic profiling. A patient having a CD-PBmu profile may express one or more genes of Tables 1A-1B at a level higher than a reference subject that does not have CD or a CD-PBmu profile. The one or more genes may comprise one or more genes of Table 1B. Patients having the CD-PBmu profile may be suitable for subtype-specific treatment, including administration with a therapeutic agent that targets a biomolecule provided in Tables 1A-1B, 3, 14, 15, 17A-17B, 20A-20B; a therapeutic agent that targets a biomolecule in a biological pathway of a biomolecule provided in Tables 1A-1B, 3, 14, 15, 17A-17B, 20A-20B; or a therapeutic agent of Tables 3-12, 20B; or a combination thereof. In some embodiments, the subtype-specific treatment comprises a therapeutic of Table 20B and/or a kinase modulator of a kinase in Table 20A. In some embodiments, the subtype-specific treatment comprises a modulator of microRNA 155 (miR-155). Non-limiting examples of miR-155 modulators include molecules that inhibit miR-155, such as Cobomarsen. Further exemplary miR-155 modulators include oligonucleotides of Tables 3-12. In some embodiments, a CD patient is characterized as having or not having a particular monocyte profile, monocyte 2 subtype. Patients having the monocyte 2 subtype may have or become susceptive to having a more severe disease phenotype. As a non-limiting example, the subject with the monocyte 2 subtype has or is likely to fail treatment with anti-TNF, 6-mercaptopurine, and/or methotrexate. Patients having the particular monocyte profile may be suitable for subtype-specific treatment, including administration with a therapeutic agent that targets a biomolecule provided in Table 17A or 17B, or a biomolecule in a biological pathway of a biomolecule provided in Table 17A or 17B. In some cases, a subject may be treated with a modulator of a kinase selected from PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, CSNK2A1, DNAPK, CDK4, ERK1, HIPK2, CDC2, MAPK3, ERK2, CSNK2A1, CK2ALPHA, JNK1, MAPK14, and PKR. Non-limiting examples of kinase targets include those in Table 20A. In some embodiments, a kinase target comprises one or more of the kinases of Table 20A. Non-limiting examples of kinase modulators includes those in Table 20B. In some embodiments, a kinase modulator comprises one or more kinase modulators of Table 20B. In some cases, the subtype-specific treatment comprises a modulator of miR-155. Non-limiting examples of miR-155 modulators include molecules that inhibit miR-155, such as Cobomarsen. Further exemplary miR-155 modulators include oligonucleotides of Tables 3-12.

Further provided herein are methods and systems for characterizing and treating a patient having CD, wherein the patient is characterized as having or not having a CD-PBmu subtype, and having or not having a monocyte 2 subtype. The non-CD-PBmu subtype may be a PBT subtype. The non-monocyte 2 subtype may be a monocyte 1 subtype. The subtype characterization may be determined sequentially or concurrently. In some cases, a patient having a CD-PBmu subtype and monocyte 2 subtype is treated with a therapeutic agent that targets a biomolecule provided in Table 1A, 1B, 14, 17A, 17B, PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, or PKR. In some cases, a patient having a CD-PBmu subtype and monocyte 2 subtype is treated with a modulator of a kinase of Table 20A. In some cases, a patient having a CD-PBmu subtype and monocyte 2 subtype is treated with an agent of Table 20A. In some cases, a patient having a CD-PBmu subtype and monocyte 2 subtype is treated with a modulator of miR-155. Non-limiting examples of miR-155 modulators include molecules that inhibit miR-155, such as Cobomarsen. Further exemplary miR-155 modulators include oligonucleotides of Tables 3-12. In some cases, a patient having a CD-PBmu subtype and monocyte 2 subtype is not treated with anti-TNF, 6-mercaptopurine, or methotrexate. In some cases, a patient having a CD-PBmu subtype and monocyte 1 subtype is treated with a therapeutic agent that targets a biomolecule provided in Table 1A, 1B, 14, 17A, 17B, PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, or PKR. In some cases, a patient having a CD-PBmu subtype and monocyte 1 subtype is treated with a modulator of a kinase of Table 20A. In some cases, a patient having a CD-PBmu subtype and monocyte 1 subtype is treated with an agent of Table 20A. In some cases, a patient having a CD-PBmu subtype and monocyte 1 subtype is treated with a modulator of miR-155. Non-limiting examples of miR-155 modulators include molecules that inhibit miR-155, such as Cobomarsen. Further exemplary miR-155 modulators include oligonucleotides of Tables 3-12. In some cases, a patient having a CD-PBmu subtype and monocyte 1 subtype is not treated with anti-TNF, 6-mercaptopurine, or methotrexate. In some cases, a patient having a CD-PBT subtype and monocyte 2 subtype is treated with a therapeutic agent that targets a biomolecule provided in Table 1A, 1B, 14, 17A, 17B, PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, or PKR. In some cases, a patient having a CD-PBT subtype and monocyte 2 subtype is treated with a modulator of a kinase of Table 20A. In some cases, a patient having a CD-PBT subtype and monocyte 2 subtype is treated with an agent of Table 20A. In some cases, a patient having a CD-PBT subtype and monocyte 2 subtype is treated with a modulator of miR-155. Non-limiting examples of miR-155 modulators include molecules that inhibit miR-155, such as Cobomarsen. Further exemplary miR-155 modulators include oligonucleotides of Tables 3-12. In some cases, a patient having a CD-PBT subtype and monocyte 2 subtype is not treated with anti-TNF, 6-mercaptopurine, or methotrexate. In some cases, a patient having a CD-PBT subtype and monocyte 1 subtype is treated with a therapeutic agent that targets a biomolecule provided in Table 1A, 1B, 14, 17A, 17B, PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, or PKR. In some cases, a patient having a CD-PBT subtype and monocyte 1 subtype is treated with a modulator of a kinase of Table 20A. In some cases, a patient having a CD-PBT subtype and monocyte 1 subtype is treated with an agent of Table 20A. In some cases, a patient having a CD-PBT subtype and monocyte 1 subtype is treated with a modulator of miR-155. Non-limiting examples of miR-155 modulators include molecules that inhibit miR-155, such as Cobomarsen. Further exemplary miR-155 modulators include oligonucleotides of Tables 3-12. In some cases, a patient having a CD-PBT subtype and monocyte 1 subtype is not treated with anti-TNF, 6-mercaptopurine, or methotrexate.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

### Transcriptomic Signature and Profiling

In one aspect, provided herein are transcriptomic signatures associated with a subtype of IBD, including CD. In some cases, the transcriptomic signature comprises one or more genes of Table 1. As used herein, Table 1 is inclusive of Table 1A and Table 1B. In some cases, the transcriptomic signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1. In some cases, the transcriptomic signature comprises genes 1-44 of Table 1. In some cases, the transcriptomic signature comprises genes 1-117 of Table 1. In some cases, the transcriptomic signature comprises one or more genes of Table 1A. In some cases, the transcriptomic signature comprises one or more genes of Table 1B. In some embodiments, the subtype is associated with perianal disease/fistula, stricturing disease, recurrence, or increased immune reactivity to a microbial antigen, or a combination thereof.

TABLE 1A
Exemplary Biomarkers of a Transcriptomic Signature

| No | Biomarker | Name | EntrezID | Accession | UGCluster | Ensembl |
|---|---|---|---|---|---|---|
| 1 | ADAM28 | ADAM | 10863 | NM_001304351 | Hs.174030 | ENSG00000042980 |
| | | metallopeptidase | | | | |
| | | domain 28 | | | | |
| 2 | ADAMDEC1 | ADAM-like, | 27299 | NM_001145271 | Hs.521459 | ENSG00000134028 |
| | | decysin 1 | | | | |
| 3 | ADAMTS1 | ADAM | 9510 | NM_006988 | Hs.643357 | ENSG00000154734 |
| | | metallopeptidase | | | | |
| | | with | | | | |
| | | thrombospondin | | | | |
| | | type 1 motif, 1 | | | | |
| 4 | ALDOB | aldolase B, | 229 | NM_000035 | Hs.530274 | ENSG00000136872 |
| | | fructose- | | | | |
| | | bisphosphate | | | | |
| 5 | C1S | complement | 716 | NM_001734 | Hs.458355 | ENSG00000182326 |
| | | component 1, s | | | | |
| | | subcomponent | | | | |
| 6 | CHAC1 | ChaC | 79094 | NM_001142776 | Hs.155569 | ENSG00000128965 |
| | | glutathione- | | | | |
| | | specific gamma- | | | | |
| | | glutamylcyclotransferase 1 | | | | |
| 7 | CHST15 | carbohydrate (N- | 51363 | NM_001270764 | Hs.287537 | ENSG00000182022 |
| | | acetylgalactosamine | | | | |
| | | 4-sulfate 6-O) | | | | |
| | | sulfotransferase 15 | | | | |
| 8 | CPA3 | carboxypeptidase | 1359 | NM_001870 | Hs.646 | ENSG00000163751 |
| | | A3 (mast cell) | | | | |
| 9 | CRYAB | crystallin, alpha B | 1410 | NM_001289807 | Hs.53454 | ENSG00000109846 |
| 10 | DAB2 | Dab, mitogen- | 1601 | NM_001244871 | Hs.696631 | ENSG00000153071 |
| | | responsive | | | | |
| | | phosphoprotein, | | | | |
| | | homolog 2 | | | | |
| | | (Drosophila) | | | | |
| 11 | DCN | decorin | 1634 | NM_001920 | Hs.156316 | ENSG00000011465 |
| 12 | DSE | dermatan sulfate | 29940 | NM_001080976 | Hs.458358 | ENSG00000111817 |
| | | epimerase | | | | |
| 13 | DYRK3 | dual-specificity | 8444 | NM_001004023 | Hs.164267 | ENSG00000143479 |
| | | tyrosine-(Y)- | | | | |
| | | phosphorylation | | | | |
| | | regulated kinase 3 | | | | |
| 14 | FABP1 | fatty acid binding | 2168 | NM_001443 | Hs.380135 | ENSG00000163586 |
| | | protein 1, liver | | | | |
| 15 | FPR3 | formyl peptide | 2359 | NM_002030 | Hs.445466 | ENSG00000187474 |
| | | receptor 3 | | | | |
| 16 | GFPT2 | glutamine- | 9945 | NM_005110 | Hs.696497 | ENSG00000131459 |
| | | fructose-6- | | | | |
| | | phosphate | | | | |
| | | transaminase 2 | | | | |
| 17 | HDC | histidine | 3067 | NM_001306146 | Hs.1481 | ENSG00000140287 |
| | | decarboxylase | | | | |
| 18 | IL22 | interleukin 22 | 50616 | NM_020525 | Hs.287369 | ENSG00000127318 |
| 19 | IL6 | interleukin 6 | 3569 | NM_000600 | Hs.654458 | ENSG00000136244 |
| 20 | KIT | v-kit Hardy- | 3815 | NM_000222 | Hs.479754 | ENSG00000157404 |
| | | Zuckerman 4 | | | | |
| | | feline sarcoma | | | | |
| | | viral oncogene | | | | |
| | | homolog | | | | |
| 21 | LCN2 | lipocalin 2 | 3934 | NM_005564 | Hs.204238 | ENSG00000148346 |
| 22 | LMCD1 | LIM and | 29995 | NM_001278233 | Hs.475353 | ENSG00000071282 |
| | | cysteine-rich | | | | |
| | | domains 1 | | | | |
| 23 | LRRC32 | leucine rich | 2615 | NM_001128922 | Hs.151641 | ENSG00000137507 |
| | | repeat containing 32 | | | | |
| 24 | LYZ | lysozyme | 4069 | NM_000239 | Hs.524579 | ENSG00000090382 |
| 25 | MFSD2A | major facilitator | 84879 | NM_001136493 | Hs.655177 | ENSG00000168389 |
| | | superfamily | | | | |
| | | domain | | | | |
| | | containing 2A | | | | |
| 26 | NANOGNB | NANOG | 360030 | NM_001145465 | Hs.558004 | ENSG00000205857 |
| | | neighbor | | | | |
| | | homeobox | | | | |
| 27 | OR4A5 | olfactory | 81318 | NM_001005272 | Hs.554531 | ENSG00000221840 |
| | | receptor, family | | | | |
| | | 4, subfamily A, | | | | |
| | | member 5 | | | | |
| 28 | PLA2G2A | phospholipase | 5320 | NM_000300 | Hs.466804 | ENSG00000188257 |
| | | A2, group IIA | | | | |
| | | (platelets, | | | | |
| | | synovial fluid) | | | | |
| 29 | PLTP | phospholipid | 5360 | NM_001242920 | Hs.439312 | ENSG00000100979 |
| | | transfer protein | | | | |
| 30 | PPIAP30 | peptidylproly1 | 100192204 | NR_036506 | Hs.714691 | |
| | | isomerase A | | | | |
| | | (cyclophilin A) | | | | |
| | | pseudogene 30 | | | | |
| 31 | RAB13 | RAB13, member | 5872 | NM_001272038 | Hs.151536 | ENSG00000143545 |
| | | RAS oncogene | | | | |
| | | family | | | | |
| 32 | RRAD | Ras-related | 6236 | NM_00112885-0 | Hs.1027 | ENSG00000166592 |
| | | associated with | | | | |
| | | diabetes | | | | |
| 33 | SDS | serine | 10993 | NM_006843 | Hs.439023 | ENSG00000135094 |
| | | dehydratase | | | | |
| 34 | SEMA6B | sema domain, | 10501 | NM_020241 | Hs.465642 | ENSG00000167680 |
| | | transmembrane | | | | |
| | | domain, and | | | | |
| | | cytoplasmic | | | | |
| | | domain, | | | | |
| | | (semaphorin) 6B | | | | |
| 35 | SEPP1 | selenoprotein P, | 6414 | NM_001085486 | Hs.275775 | ENSG00000250722 |
| | | plasma, 1 | | | | |
| 36 | SERPING1 | serpin peptidase | 710 | NM_000062 | Hs.384598 | ENSG00000149131 |
| | | inhibitor, clade G | | | | |
| | | (C1 inhibitor), | | | | |
| | | member 1 | | | | |
| 37 | SOD3 | superoxide | 6649 | NM_003102 | Hs.2420 | ENSG00000109610 |
| | | dismutase 3, | | | | |
| | | extracellular | | | | |
| 38 | SYK | spleen tyrosine | 6850 | NM_001135052 | Hs.371720 | ENSG00000165025 |
| | | kinase | | | | |
| 39 | TBC1D3 | TBC1 domain | 729873 | NM_001123391 | Hs.454716 | ENSG00000274611 |
| | | family, member 3 | | | | |
| 40 | TBC1D8 | TBC1 domain | 11138 | NM_001102426 | Hs.442657 | ENSG00000204634 |
| | | family, member 8 | | | | |
| | | (with GRAM | | | | |
| | | domain) | | | | |
| 41 | TBC1D9 | TBC1 domain | 23158 | NM_015130 | Hs.480819 | ENSG00000109436 |
| | | family, member 9 | | | | |
| | | (with GRAM | | | | |
| | | domain) | | | | |
| 42 | TNXB | tenascin XB | 7148 | NM_019105 | Hs.485104 | ENSG00000168477 |
| 43 | TPSB2 | tryptase beta 2 | 64499 | NM_024164 | Hs.405479 | ENSG00000197253 |
| | | (gene/pseudogene) | | | | |
| 44 | UBD | ubiquitin D | 10537 | NM_006398 | Hs.44532 | ENSG00000213886 |
| 45 | ABI3BP | ABI family, | 25890 | NM_015429 | Hs.477015 | ENSG00000154175 |
| | | member 3 | | | | |
| | | (NESH) binding | | | | |
| | | protein | | | | |
| 46 | ANKRD20A3 | ankyrin repeat | 441425 | NM_001012419 | Hs.632663 | ENSG00000276203 |
| | | domain 20 | | | | |
| | | family, member A3 | | | | |
| 47 | APOC1P1 | apolipoprotein C- | 342 | NR_028412 | Hs.110675 | ENSG00000214855 |
| | | I pseudogene 1 | | | | |
| 48 | AQP7P3 | aquaporin 7 | 441432 | NR_026558 | Hs.743215 | |
| | | pseudogene 3 | | | | |
| 49 | C11orf96 | chromosome 11 | 387763 | NM_001145033 | Hs.530443 | ENSG00000187479 |
| | | open reading | | | | |
| | | frame 96 | | | | |
| 50 | C1QB | complement | 713 | NM_000491 | Hs.8986 | ENSG00000173369 |
| | | component 1, q | | | | |
| | | subcomponent, B | | | | |
| | | chain | | | | |
| 51 | C1QC | complement | 714 | NM_001114101 | Hs.467753 | ENSG00000159189 |
| | | component 1, q | | | | |
| | | subcomponent, C | | | | |
| | | chain | | | | |
| 52 | C2orf27A | chromosome 2 | 29798 | NM_013310 | Hs.635289 | |
| | | open reading | | | | |
| | | frame 27A | | | | |
| 53 | C8orf4 | chromosome 8 | 56892 | NM_020130 | Hs.591849 | ENSG00000176907 |
| | | open reading | | | | |
| | | frame 4 | | | | |
| 54 | CKB | creatine kinase, | 1152 | NM_001823 | Hs.173724 | ENSG00000166165 |
| | | brain | | | | |
| 55 | CLDN10 | claudin 10 | 9071 | NM_001160100 | Hs.534377 | ENSG00000134873 |
| 56 | CLEC3B | C-type lectin | 7123 | NM_001308394 | Hs.476092 | ENSG00000163815 |
| | | domain family 3, | | | | |
| | | member B | | | | |
| 57 | CLIC4 | chloride | 25932 | NM_013943 | Hs.440544 | ENSG00000169504 |
| | | intracellular | | | | |
| | | channel 4 | | | | |
| 58 | COL1A1 | collagen, type I, | 1277 | NM_000088 | Hs.172928 | ENSG00000108821 |
| | | alpha 1 | | | | |
| 59 | COL1A2 | collagen, type I, | 1278 | NM_000089 | Hs.489142 | ENSG00000164692 |
| | | alpha 2 | | | | |
| 60 | COL5A1 | collagen, type V, | 1289 | NM_000093 | Hs.210283 | ENSG00000130635 |
| | | alpha 1 | | | | |
| 61 | CXCL13 | chemokine (C-X- | 10563 | NM_006419 | Hs.100431 | ENSG00000156234 |
| | | C motif) ligand 13 | | | | |
| 62 | CYCSP52 | cytochrome c, | 360155 | NR_001560 | Hs.491808 | ENSG00000235700 |
| | | somatic | | | | |
| | | pseudogene 52 | | | | |
| 63 | FAM138D | family with | 677784 | NR_026823 | Hs.722487 | ENSG00000249054 |
| | | sequence | | | | |
| | | similarity 138, | | | | |
| | | member D | | | | |
| 64 | FAM182B | family with | 728882 | NR_026714 | Hs.682103 | ENSG00000175170 |
| | | sequence | | | | |
| | | similarity 182, | | | | |
| | | member B | | | | |
| 65 | FAM222A | family with | 84915 | NM_032829 | Hs.661785 | ENSG00000139438 |
| | | sequence | | | | |
| | | similarity 222, | | | | |
| | | member A | | | | |
| 66 | FAM231A | family with | 729574 | NM_001282321 | | ENSG00000237847 |
| | | sequence | | | | |
| | | similarity 231, | | | | |
| | | member A | | | | |
| 67 | FAM27A | | | | | |
| 68 | FSTL1 | follistatin-like 1 | 11167 | NM_007085 | Hs.269512 | ENSG00000163430 |
| 69 | GAS7 | growth arrest- | 8522 | NM_001130831 | Hs.462214 | ENSG00000007237 |
| | | specific 7 | | | | |
| 70 | GEM | GTP binding | 2669 | NM_005261 | Hs.654463 | ENSG00000164949 |
| | | protein | | | | |
| | | overexpressed in | | | | |
| | | skeletal muscle | | | | |
| 71 | GOLGA6L5P | golgin A6 | 374650 | NM_198079 | Hs.454625 | |
| | | family-like 5, | | | | |
| | | pseudogene | | | | |
| 72 | GPNMB | glycoprotein | 10457 | NM_001005340 | Hs.190495 | ENSG00000136235 |
| | | (transmembrane) | | | | |
| | | nmb | | | | |
| 73 | GYPE | glycophorin E | 2996 | NM_002102 | Hs.654368 | ENSG00000197465 |
| | | (MNS blood | | | | |
| | | group) | | | | |
| 74 | HNRNPA1P33 | heterogeneous | 728643 | NR_003277 | Hs.711067 | ENSG00000213412 |
| | | nuclear | | | | |
| | | ribonucleoprotein | | | | |
| | | A1 pseudogene 33 | | | | |
| 75 | HSPA2 | heat shock 70 kDa | 3306 | NM_021979 | Hs.432648 | ENSG00000126803 |
| | | protein 2 | | | | |
| 76 | HSPB6 | heat shock | 126393 | NM_144617 | Hs.534538 | ENSG00000004776 |
| | | protein, alpha- | | | | |
| | | crystallin-related, | | | | |
| | | B6 | | | | |
| 77 | KGFLP2 | keratinocyte | 654466 | NM_001039113 | Hs.536967 | |
| | | growth factor- | | | | |
| | | like protein 2 | | | | |
| 78 | KRT20 | keratin 20, type I | 54474 | NM_019010 | Hs.84905 | ENSG00000171431 |
| 79 | LIMS3L | LIM and | 100288695 | NM_001205288 | Hs.535619 | ENSG00000256671 |
| | | senescent cell | | | | |
| | | antigen-like | | | | |
| | | domains 3-like | | | | |
| 80 | LINC00348 | long intergenic | 100885781 | NR_047699 | Hs.372660 | ENSG00000226846 |
| | | non-protein | | | | |
| | | coding RNA 348 | | | | |
| 81 | LINC00700 | long intergenic | 282980 | NR_040253 | Hs.576810 | ENSG00000234962 |
| | | non-protein | | | | |
| | | coding RNA 700 | | | | |
| 82 | LINC00857 | long intergenic | 439990 | NR_038464 | Hs.365566 | ENSG00000237523 |
| | | non-protein | | | | |
| | | coding RNA 857 | | | | |
| 83 | LINC01189 | long intergenic | 643648 | NR_046203 | Hs.640178 | |
| | | non-protein | | | | |
| | | coding RNA | | | | |
| | | 1189 | | | | |
| 84 | LOC100129138 | THAP domain | 100129138 | NR_033990 | Hs.514487 | ENSG00000215869 |
| | | containing, | | | | |
| | | apoptosis | | | | |
| | | associated protein | | | | |
| | | 3 pseudogene | | | | |
| 85 | LOC100507006 | uncharacterized | 100507006 | NR_120420 | Hs.442789 | |
| | | LOC100507006 | | | | |
| 86 | LOC100508046 | uncharacterized | 100508046 | NR_110505 | Hs.433218 | ENSG00000275563 |
| | | LOC100508046 | | | | |
| 87 | LOC101927123 | uncharacterized | 101927123 | NR_110147 | Hs.526761 | ENSG00000244215 |
| | | LOC101927123 | | | | |
| 88 | LOC101927905 | uncharacterized | 101927905 | NR_120454 | Hs.621425 | ENSG00000215241 |
| | | LOC101927905 | | | | |
| 89 | LOC101928163 | uncharacterized | 101928163 | NR_110799 | Hs.588761 | |
| | | LOC101928163 | | | | |
| 90 | LOC102724034 | uncharacterized | 102724034 | NR_120378 | Hs.694638 | |
| | | LOC102724034 | | | | |
| 91 | LOC642426 | uncharacterized | 642426 | NR_046104 | Hs.578301 | ENSG00000257504 |
| | | LOC642426 | | | | |
| 92 | LOC645166 | lymphocyte- | 645166 | NR_027354 | Hs.744183 | ENSG00000232527 |
| | | specific protein 1 | | | | |
| | | pseudogene | | | | |
| 93 | LOC646736 | uncharacterized | 646736 | NR_046102 | Hs.712836 | |
| | | LOC646736 | | | | |
| 94 | MIR663A | microRNA 663a | 724033 | NR_030386 | | ENSG00000273684 |
| 95 | MLLT10P1 | myeloid/lymphoid | 140678 | NR_045115 | Hs.653099 | |
| | | or mixed- | | | | |
| | | lineage leukemia; | | | | |
| | | translocated to, | | | | |
| | | 10 pseudogene 1 | | | | |
| 96 | MMP19 | matrix | 4327 | NM_001032360 | Hs.591033 | ENSG00000123342 |
| | | metallopeptidase 19 | | | | |
| 97 | NCOR1P1 | nuclear receptor | 149934 | NM_001039379 | Hs.711274 | ENSG00000240108 |
| | | corepressor 1 | | | | |
| | | pseudogene 1 | | | | |
| 98 | PGM5-AS1 | PGM5 antisense | 572558 | NR_015423 | Hs.552819 | ENSG00000224958 |
| | | RNA 1 | | | | |
| 99 | PHLDB1 | pleckstrin | 23187 | NM_001144758 | Hs.504062 | ENSG00000019144 |
| | | homology-like | | | | |
| | | domain, family | | | | |
| | | B, member 1 | | | | |
| 100 | PMP22 | peripheral myelin | 5376 | NM_000304 | Hs.372031 | ENSG0000010909 |
| | | protein 22 | | | | |
| 101 | PTENP1-AS | PTENP1 | 101243555 | NR_103745 | Hs.598470 | ENSG00000281128 |
| | | antisense RNA | | | | |
| 102 | REG3A | regenerating | 5068 | NM_002580 | Hs.567312 | ENSG00000172016 |
| | | islet-derived 3 | | | | |
| | | alpha | | | | |
| 103 | RPSAP9 | ribosomal protein | 653162 | NR_026890 | Hs.655646 | ENSG00000234618 |
| | | SA pseudogene 9 | | | | |
| 104 | SEPSECS-AS1 | SEPSECS | 285540 | NR_037934 | Hs.732278 | |
| | | antisense RNA 1 | | | | |
| | | (head to head) | | | | |
| 105 | SEPT14 | | | | | |
| 106 | SLC9B1 | solute carrier | 150159 | NM_001100874 | Hs.666728 | ENSG00000164037 |
| | | family 9, | | | | |
| | | subfamily B | | | | |
| | | (NHA1, cation | | | | |
| | | proton antiporter | | | | |
| | | 1), member 1 | | | | |
| 107 | SLCO4A1 | solute carrier | 28231 | NM_016354 | Hs.235782 | ENSG00000101187 |
| | | organic anion | | | | |
| | | transporter | | | | |
| | | family, member | | | | |
| | | 4A1 | | | | |
| 108 | SMOX | spermine oxidase | 54498 | NM_001270691 | Hs.433337 | ENSG00000088826 |
| 109 | SPARCL1 | SPARC-like 1 | 8404 | NM_001128310 | Hs.62886 | ENSG00000152583 |
| | | (hevin) | | | | |
| 110 | SRC | SRC proto- | 6714 | NM_005417 | Hs.195659 | ENSG00000197122 |
| | | oncogene, non- | | | | |
| | | receptor tyrosine | | | | |
| | | kinase | | | | |
| 111 | ST13P4 | suppression of | 145165 | NM_153290 | Hs.511834 | |
| | | tumorigenicity 13 | | | | |
| | | (colon | | | | |
| | | carcinoma) | | | | |
| | | (Hsp70 | | | | |
| | | interacting | | | | |
| | | protein) | | | | |
| | | pseudogene 4 | | | | |
| 112 | TCF21 | transcription | 6943 | NM_003206 | Hs.78061 | ENSG00000118526 |
| | | factor 21 | | | | |
| 113 | TCF4 | transcription | 6925 | NM_001083962 | Hs.605153 | ENSG00000196628 |
| | | factor 4 | | | | |
| 114 | TMEM45B | transmembrane | 120224 | NM_138788 | Hs.504301 | ENSG00000151715 |
| | | protein 45B | | | | |
| 115 | UBE2Q2L | ubiquitin- | 100505679 | NM_001243531 | Hs.726826 | ENSG00000259511 |
| | | conjugating | | | | |
| | | enzyme E2Q | | | | |
| | | family member | | | | |
| | | 2-like | | | | |
| 116 | UBTFL1 | upstream binding | 642623 | NM_001143975 | Hs.719885 | ENSG00000255009 |
| | | transcription | | | | |
| | | factor, RNA | | | | |
| | | polymerase I-like 1 | | | | |
| 117 | ZNF582-AS1 | ZNF582 | 386758 | NR_037159 | Hs.549564 | ENSG00000267454 |
| | | antisense RNA 1 | | | | |
| | | (head to head) | | | | |
| 118 | ADM | adrenomedullin | 133 | NM_001124 | Hs.441047 | ENSG00000148926 |
| 119 | ANPEP | alanyl | 290 | NM_001150 | Hs.1239 | ENSG00000166825 |
| | | (membrane) | | | | |
| | | aminopeptidase | | | | |
| 120 | AOAH-IT1 | AOAH intronic | 100874264 | NR_046764 | Hs.690994 | ENSG00000230539 |
| | | transcript 1 | | | | |
| 121 | ASB2 | ankyrin repeat | 51676 | NM_001202429 | Hs.510327 | ENSG00000100628 |
| | | and SOCS box | | | | |
| | | containing 2 | | | | |
| 122 | ATP5J2- | ATP5J2-PTCD1 | 100526740 | NM_001198879 | Hs.632313 | ENSG00000248919 |
| | PTCD1 | readthrough | | | | |
| 123 | BASP1 | brain abundant, | 10409 | NM_001271606 | Hs.201641 | ENSG00000176788 |
| | | membrane | | | | |
| | | attached signal | | | | |
| | | protein 1 | | | | |
| 124 | CCL11 | chemokine (C-C | 6356 | NM_002986 | Hs.54460 | ENSG00000172156 |
| | | motif) ligand 11 | | | | |
| 125 | CD68 | CD68 molecule | 968 | NM_001040059 | Hs.647419 | ENSG00000129226 |
| 126 | CSF2RB | colony | 1439 | NM_000395 | Hs.592192 | ENSG00000100368 |
| | | stimulating factor | | | | |
| | | 2 receptor, beta, | | | | |
| | | low-affinity | | | | |
| | | (granulocyte- | | | | |
| | | macrophage) | | | | |
| 127 | CTAGE8 | CTAGE family, | 100142659 | NM_001278507 | Hs.661442 | ENSG00000244693 |
| | | member 8 | | | | |
| 128 | CTGF | connective tissue | 1490 | NM_001901 | Hs.410037 | ENSG00000118523 |
| | | growth factor | | | | |
| 129 | CXCL1 | chemokine (C-X-C | 2919 | NM_001511 | Hs.789 | ENSG00000163739 |
| | | motif) ligand 1 | | | | |
| | | (melanoma | | | | |
| | | growth | | | | |
| | | stimulating | | | | |
| | | activity, alpha | | | | |
| 130 | CXCL3 | chemokine (C-X-C | 2921 | NM_002090 | Hs.89690 | ENSG00000163734 |
| | | motif) ligand 3 | | | | |
| 131 | DEFA5 | defensin, alpha 5, | 1670 | NM_021010 | Hs.655233 | ENSG00000164816 |
| | | Paneth cell- | | | | |
| | | specific | | | | |
| 132 | DEFA6 | defensin, alpha 6, | 1671 | NM_001926 | Hs.711 | ENSG00000164822 |
| | | Paneth cell- | | | | |
| | | specific | | | | |
| 133 | DERL3 | derlin 3 | 91319 | NM_001002862 | Hs.593679 | ENSG00000099958 |
| 134 | DNASE1L3 | deoxyribonuclease | 1776 | NM_001256560 | Hs.476453 | ENSG00000163687 |
| | | I-like 3 | | | | |
| 135 | DOK3 | docking protein 3 | 79930 | NM_001144875 | Hs.720849 | ENSG00000146094 |
| 136 | EGR2 | early growth | 1959 | NM_000399 | Hs.1395 | ENSG00000122877 |
| | | response 2 | | | | |
| 137 | EGR3 | early growth | 1960 | NM_001199880 | Hs.534313 | ENSG00000179388 |
| | | response 3 | | | | |
| 138 | EMP1 | epithelial | 2012 | NM_001423 | Hs.719042 | ENSG00000134531 |
| | | membrane | | | | |
| | | protein 1 | | | | |
| 139 | EPAS1 | endothelial PAS | 2034 | NM_001430 | Hs.468410 | ENSG00000116016 |
| | | domain protein 1 | | | | |
| 140 | FAM138A | family with | 645520 | NR_026818 | Hs.569137 | ENSG00000237613 |
| | | sequence | | | | |
| | | similarity 138, | | | | |
| | | member A | | | | |
| 141 | FAM138F | family with | 641702 | NR_026820 | Hs.569137 | ENSG00000282591 |
| | | sequence | | | | |
| | | similarity 138, | | | | |
| | | member F | | | | |
| 142 | FAM157B | family with | 100132403 | NM_001145249 | Hs.741123 | |
| | | sequence | | | | |
| | | similarity 157, | | | | |
| | | member B | | | | |
| 143 | FDCSP | follicular | 260436 | NM_152997 | Hs.733448 | ENSG00000181617 |
| | | dendritic cell | | | | |
| | | secreted protein | | | | |
| 144 | FOSL1 | FOS-like antigen 1 | 8061 | NM_001300844 | Hs.283565 | ENSG00000175592 |
| 145 | FSCN1 | fascin actin- | 6624 | NM_003088 | Hs.118400 | ENSG00000075618 |
| | | bundling protein 1 | | | | |
| 146 | FTH1P3 | ferritin, heavy | 2498 | NR_002201 | Hs.658438 | |
| | | polypeptide 1 | | | | |
| | | pseudogene 3 | | | | |
| 147 | GAS6 | growth arrest- | 2621 | NM_000820 | Hs.646346 | ENSG00000183087 |
| | | specific 6 | | | | |
| 148 | GATA2 | GATA binding | 2624 | NM_001145661 | Hs.367725 | ENSG00000179348 |
| | | protein 2 | | | | |
| 149 | GPX3 | glutathione | 2878 | NM_002084 | Hs.386793 | ENSG00000211445 |
| | | peroxidase 3 | | | | |
| 150 | HES1 | hes family bHLH | 3280 | NM_005524 | Hs.250666 | ENSG00000114315 |
| | | transcription | | | | |
| | | factor 1 | | | | |
| 151 | HES4 | hes family bHLH | 57801 | NM_001142467 | Hs.154029 | ENSG00000188290 |
| | | transcription | | | | |
| | | factor 4 | | | | |
| 152 | HLA-L | major | 3139 | NR_027822 | Hs.656020 | ENSG00000243753 |
| | | histocompatibility | | | | |
| | | complex, class | | | | |
| | | I, L (pseudogene) | | | | |
| 153 | IGFBP7 | insulin-like | 3490 | NM_001253835 | Hs.479808 | ENSG00000163453 |
| | | growth factor | | | | |
| | | binding protein 7 | | | | |
| 154 | IL1RN | interleukin 1 | 3557 | NM_000577 | Hs.81134 | ENSG00000136689 |
| | | receptor | | | | |
| | | antagonist | | | | |
| 155 | IL21R-AS1 | IL21R antisense | 283888 | NR_037158 | Hs.660935 | ENSG00000259954 |
| | | RNA 1 | | | | |
| 156 | LINC01194 | long intergenic | 404663 | NR_033383 | Hs.552273 | |
| | | non-protein | | | | |
| | | coding RNA | | | | |
| | | 1194 | | | | |
| 157 | LOC100240735 | uncharacterized | 100240735 | NR_026658 | Hs.635297 | ENSG00000250654 |
| | | LOC100240735 | | | | |
| 158 | LOC101927817 | uncharacterized | 101927817 | NR_110931 | Hs.667942 | |
| | | LOC101927817 | | | | |
| 159 | LOC284801 | | | | | |
| 160 | LOC285740 | uncharacterized | 285740 | NR_027113 | Hs.432656 | ENSG00000235740 |
| | | LOC285740 | | | | |
| 161 | LOC441242 | uncharacterized | 441242 | NM_001013464 | Hs.373941 | ENSG00000272693 |
| | | LOC441242 | | | | |
| 162 | LOC644172 | mitogen- | 644172 | NR_026901 | Hs.448859 | |
| | | activated protein | | | | |
| | | kinase 8 | | | | |
| | | interacting | | | | |
| | | protein | | | | |
| | | pseudogene | | | | |
| 163 | MAFF | v-maf avian | 23764 | NM_001161572 | Hs.517617 | ENSG00000185022 |
| | | musculoaponeuro | | | | |
| | | tic fibrosarcoma | | | | |
| | | oncogene | | | | |
| | | homolog F | | | | |
| 164 | MARCKS | myristoylated | 4082 | NM_002356 | Hs.519909 | ENSG00000277443 |
| | | alanine-rich | | | | |
| | | protein kinase C | | | | |
| | | substrate | | | | |
| 165 | MCTP1 | multiple C2 | 79772 | NM_001002796 | Hs.591248 | ENSG00000175471 |
| | | domains, | | | | |
| | | transmembrane 1 | | | | |
| 166 | MGP | matrix Gla | 4256 | NM_000900 | Hs.365706 | ENSG00000111341 |
| | | protein | | | | |
| 167 | MIR548I1 | microRNA 548i-1 | 100302204 | NR_031687 | | ENSG00000221737 |
| 168 | MIR663B | microRNA 663b | 100313824 | NR_031608 | | ENSG00000221288 |
| 169 | MMP9 | matrix | 4318 | NM_004994 | Hs.297413 | ENSG00000100985 |
| | | metallopeptidase 9 | | | | |
| 170 | MT1G | metallothionein 1G | 4495 | NM_001301267 | Hs.433391 | ENSG00000125144 |
| 171 | NPIPB9 | nuclear pore | 100507607 | NM_001287250 | Hs.710214 | ENSG00000196993 |
| | | complex | | | | |
| | | interacting | | | | |
| | | protein family, | | | | |
| | | member B9 | | | | |
| 172 | NUCB1-AS1 | NUCB1 antisense | 100874085 | NR_046633 | Hs.569933 | ENSG00000235191 |
| | | RNA 1 | | | | |
| 173 | OR4F21 | olfactory | 441308 | NM_001005504 | Hs.690459 | ENSG00000176269 |
| | | receptor, family | | | | |
| | | 4, subfamily F, | | | | |
| | | member 21 | | | | |
| 174 | PHACTR1 | phosphatase and | 221692 | NM_001242648 | Hs.436996 | ENSG00000112137 |
| | | actin regulator 1 | | | | |
| 175 | PLEKHA4 | pleckstrin | 57664 | NM_001161354 | Hs.9469 | ENSG00000105559 |
| | | homology | | | | |
| | | domain | | | | |
| | | containing, | | | | |
| | | family A | | | | |
| | | (phosphoinositide | | | | |
| | | binding specific) | | | | |
| | | member 4 | | | | |
| 176 | PLGLB1 | plasminogen-like B1 | 5343 | NM_001032392 | Hs.652169 | ENSG00000183281 |
| 177 | POC1B- | POC1B- | 100528030 | NM_001199781 | Hs.25130 | ENSG00000259075 |
| | GALNT4 | GALNT4 | | | | |
| | | readthrough | | | | |
| 178 | PRKX-AS1 | PRKX antisense | 100873944 | NR_046643 | | ENSG00000236188 |
| | | RNA 1 | | | | |
| 179 | PTGS2 | prostaglandin- | 5743 | NM_000963 | Hs.196384 | ENSG00000073756 |
| | | endoperoxide | | | | |
| | | synthase 2 | | | | |
| | | (prostaglandin | | | | |
| | | G/H synthase and | | | | |
| | | cyclooxygenase) | | | | |
| 180 | RAB20 | RAB20, member | 55647 | NM_017817 | Hs.743563 | ENSG00000139832 |
| | | RAS oncogene | | | | |
| | | family | | | | |
| 181 | REGIA | regenerating | 5967 | NM_002909 | Hs.49407 | ENSG00000115386 |
| | | islet-derived 1 | | | | |
| | | alpha | | | | |
| 182 | RNASE1 | ribonuclease, | 6035 | NM_002933 | Hs.78224 | ENSG00000129538 |
| | | RNase A family, | | | | |
| | | 1 (pancreatic) | | | | |
| 183 | SDC4 | syndecan 4 | 6385 | NM_002999 | Hs.632267 | ENSG00000124145 |
| 184 | SEPT10 | | | | | |
| 185 | SIRPA | signal-regulatory | 140885 | NM_001040022 | Hs.581021 | ENSG00000198053 |
| | | protein alpha | | | | |
| 186 | SNAI1 | snail family zinc | 6615 | NM_005985 | Hs.48029 | ENSG00000124216 |
| | | finger 1 | | | | |
| 187 | SPARC | secreted protein, | 6678 | NM_001309443 | Hs.111779 | ENSG00000113140 |
| | | acidic, cysteine- | | | | |
| | | rich (osteonectin) | | | | |
| 188 | SPHK1 | sphingosine | 8877 | NM_001142601 | Hs.68061 | ENSG00000176170 |
| | | kinase 1 | | | | |
| 189 | SPINK4 | serine peptidase | 27290 | NM_014471 | Hs.555934 | ENSG00000122711 |
| | | inhibitor, Kazal | | | | |
| | | type 4 | | | | |
| 190 | STAB1 | stabilin 1 | 23166 | NM_015136 | Hs.301989 | ENSG00000010327 |
| 191 | TMEM114 | transmembrane | 283953 | NM_001146336 | Hs.150849 | ENSG00000232258 |
| | | protein 114 | | | | |
| 192 | TNFAIP2 | tumor necrosis | 7127 | NM_006291 | Hs.525607 | ENSG00000185215 |
| | | factor, alpha- | | | | |
| | | induced protein 2 | | | | |
| 193 | TNFRSF12A | tumor necrosis | 51330 | NM_016639 | Hs.355899 | ENSG00000006327 |
| | | factor receptor | | | | |
| | | superfamily, | | | | |
| | | member 12A | | | | |
| 194 | TNFRSF13B | tumor necrosis | 23495 | NM_012452 | Hs.158341 | ENSG00000240505 |
| | | factor receptor | | | | |
| | | superfamily, | | | | |
| | | member 13B | | | | |
| 195 | TPSAB1 | tryptase | 7177 | NM_003294 | Hs.405479 | ENSG00000172236 |
| | | alpha/beta 1 | | | | |
| 196 | TREM1 | triggering | 54210 | NM_001242589 | Hs.283022 | ENSG00000124731 |
| | | receptor | | | | |
| | | expressed on | | | | |
| | | myeloid cells 1 | | | | |
| 197 | TUBB6 | tubulin, beta 6 | 84617 | NM_001303524 | Hs.193491 | ENSG00000176014 |
| | | class V | | | | |
| 198 | UGT2B10 | UDP | 7365 | NM_001075 | Hs.201634 | ENSG00000109181 |
| | | glucuronosyltransferase | | | | |
| | | 2 family, | | | | |
| | | polypeptide B10 | | | | |
| 199 | UPK3B | uroplakin 3B | 80761 | NM_030570 | Hs.488861 | ENSG00000243566 |
| 200 | VEGFA | vascular | 7422 | NM_001025366 | Hs.73793 | ENSG00000112715 |
| | | endothelial | | | | |
| | | growth factor A | | | | |
| 392-409 | miR-155 | microRNA-155 | | NR_030784 | | ENST00000385060.1 |

TABLE 1B
Exemplary Biomarkers of a Transcriptomic Signature

| Biomarker | Name | EntrezID | Accession | UGCluster | Ensembl |
|---|---|---|---|---|---|
| ADH4 | alcohol dehydrogenase 4 | 127 | NM_000670 | Hs.1219 | ENSG00000198099 |
| | (class II), pi polypeptide | | | | |
| ALG1L | ALG1, | 200810 | NM_001015050 | Hs.591299 | ENSG00000189366 |
| | chitobiosyldiphosphodolichol | | | | |
| | beta- | | | | |
| | mannosyltransferase-like | | | | |
| BCDIN3D | BCDIN3 domain | 144233 | NM_181708 | Hs.142736 | ENSG00000186666 |
| | containing | | | | |
| C1orf106 | chromosome 1 open | 55765 | NM_001142569 | Hs.518997 | ENSG00000163362 |
| | reading frame 106 | | | | |
| C2 | complement component 2 | 717 | NM_000063 | Hs.408903 | ENSG00000166278 |
| CCDC144NL | coiled-coil domain | 339184 | NM_001004306 | Hs.674830 | ENSG00000205212 |
| | containing 144 family, N- | | | | |
| | terminal like | | | | |
| CEACAM5 | carcinoembryonic antigen- | 1048 | NM_001291484 | Hs.709196 | ENSG00000105388 |
| | related cell adhesion | | | | |
| | molecule 5 | | | | |
| CTAGE8 | CTAGE family, member 8 | 1E+08 | NM_001278507 | Hs.661442 | ENSG00000244693 |
| DDX11L2 | DEAD/H (Asp-Glu-Ala- | 84771 | NR_024004 | Hs.712940 | ENSG00000223972 |
| | Asp/His) box helicase 11 | | | | |
| | like 2 | | | | |
| DPPA4 | developmental | 55211 | NM_018189 | Hs.317659 | ENSG00000121570 |
| | pluripotency associated 4 | | | | |
| DUSP19 | dual specificity | 142679 | NM_001142314 | Hs.132237 | ENSG00000162999 |
| | phosphatase 19 | | | | |
| FGB | fibrinogen beta chain | 2244 | NM_001184741 | Hs.300774 | ENSG00000171564 |
| GP2 | glycoprotein 2 (zymogen | 2813 | NM_001007240 | Hs.53985 | ENSG00000169347 |
| | granule membrane) | | | | |
| GYPE | glycophorin E (MNS blood | 2996 | NM_002102 | Hs.654368 | ENSG00000197465 |
| | group) | | | | |
| HSD3B7 | hydroxy-delta-5-steroid | 80270 | NM_001142777 | Hs.460618 | ENSG00000099377 |
| | dehydrogenase, 3 beta- and | | | | |
| | steroid delta-isomerase 7 | | | | |
| HUNK | hormonally up-regulated | 30811 | NM_014586 | Hs.109437 | ENSG00000142149 |
| | Neu-associated kinase | | | | |
| JAM2 | junctional adhesion | 58494 | NM_001270407 | Hs.517227 | ENSG00000154721 |
| | molecule 2 | | | | |
| KCNE3 | potassium channel, voltage | 10008 | NM_005472 | Hs.523899 | ENSG00000175538 |
| | gated subfamily E | | | | |
| | regulatory beta subunit 3 | | | | |
| KRT42P | keratin 42 pseudogene | 284116 | NR_033415 | Hs.725790 | ENSG00000214514 |
| LYZ | lysozyme | 4069 | NM_000239 | Hs.524579 | ENSG00000090382 |
| MLLT10P1 | myeloid/lymphoid or | 140678 | NR_045115 | Hs.653099 | |
| | mixed-lineage leukemia; | | | | |
| | translocated to, 10 | | | | |
| | pseudogene 1 | | | | |
| NAP1L6 | nucleosome assembly | 645996 | NR_027291 | Hs.617486 | ENSG00000204118 |
| | protein 1-like 6 | | | | |
| NEURL3 | neuralized E3 ubiquitin | 93082 | NM_001080535 | Hs.149219 | ENSG00000163121 |
| | protein ligase 3 | | | | |
| NPIPB9 | nuclear pore complex | 1.01E+08 | NM_001287250 | Hs.710214 | ENSG00000196993 |
| | interacting protein family, | | | | |
| | member B9 | | | | |
| PANK1 | pantothenate kinase 1 | 53354 | NM_138316 | Hs.163555 | ENSG00000152782 |
| PKIB | protein kinase (cAMP- | 5570 | NM_001270393 | Hs.741340 | ENSG00000135549 |
| | dependent, catalytic) | | | | |
| | inhibitor beta | | | | |
| RHOU | ras homolog family | 58480 | NM_021205 | Hs.647774 | ENSG00000116574 |
| | member U | | | | |
| RPSAP9 | ribosomal protein SA | 653162 | NR_026890 | Hs.655646 | ENSG00000234618 |
| | pseudogene 9 | | | | |
| SHCBP1 | SHC SH2-domain binding | 79801 | NM_024745 | Hs.123253 | ENSG00000171241 |
| | protein 1 | | | | |
| SIGLEC8 | sialic acid binding Ig-like | 27181 | NM_014442 | Hs.447899 | ENSG00000105366 |
| | lectin 8 | | | | |
| SLC15A2 | solute carrier family 15 | 6565 | NM_001145998 | Hs.518089 | ENSG00000163406 |
| | (oligopeptide transporter), | | | | |
| | member 2 | | | | |
| SLC25A34 | solute carrier family 25, | 284723 | NM_207348 | Hs.631867 | ENSG00000162461 |
| | member 34 | | | | |
| SLC6A20 | solute carrier family 6 | 54716 | NM_020208 | Hs.413095 | ENSG00000163817 |
| | (proline IMINO | | | | |
| | transporter), member 20 | | | | |
| SLC9B1 | solute carrier family 9, | 150159 | NM_001100874 | Hs.666728 | ENSG00000164037 |
| | subfamily B (NHA1, | | | | |
| | cation proton antiporter 1), | | | | |
| | member 1 | | | | |
| SYNPO2L | synaptopodin 2-like | 79933 | NM_001114133 | Hs.645273 | ENSG00000166317 |
| TDGF1 | teratocarcinoma-derived | 6997 | NM_001174136 | Hs.385870 | ENSG00000241186 |
| | growth factor 1 | | | | |
| ZNF491 | zinc finger protein 491 | 126069 | NM_152356 | Hs.631634 | ENSG00000177599 |
| ZNF620 | zinc finger protein 620 | 253639 | NM_001256167 | Hs.581541 | ENSG00000177842 |
| ZNF69 | zinc finger protein 69 | 7620 | NM_021915 | Hs.565280 | ENSG00000198429 |
| CXCL16 | chemokine (C-X-C motif) | 58191 | NM_001100812 | Hs.745037 | ENSG00000161921 |
| | ligand 16 | | | | |
| CD68 | CD68 molecule | 968 | NM_001040059 | Hs.647419 | ENSG00000129226 |
| CD300E | CD300e molecule | 342510 | NM_181449 | Hs.158954 | ENSG00000186407 |

Further provided are methods and compositions for characterizing a subtype of Crohn's Disease (CD) in a subject. A non-limiting subtype is CD-PBmu, which is associated with a mucosal-like expression profile. In some cases, the CD-PBmu subtype is associated with an altered composition of T-cell subsets, clinical disease severity markers, and decreased pro-inflammatory gene expression following surgery. In some embodiments, the PB-mu subtype is associated with perianal disease/fistula, stricturing disease, recurrence, or increased immune reactivity to a microbial antigen, or a combination thereof. The characterization methods provided include diagnosing the presence or absence of a CD subtype, prognosing whether a subject is predisposed to developing a particular CD subtype, prognosing a response of a patient with a particular CD subtype to a therapeutic treatment, and monitoring CD treatment. In some embodiments, the treatment comprises a miR-155 modulator, such as an inhibitor of miR-155. In some embodiments, the treatment comprises a modulator of a kinase, such as a kinase of Table 20A. In some embodiments, the kinase modulator comprises an agent of Table 20B.

In some embodiments, the methods involve detecting in a biological sample from a subject expression levels of one or more genes of a transcriptomic signature to obtain an expression profile comprising the expression levels of each of the one or more genes in the signature. In some embodiments, the transcriptomic signature comprises one or more biomarkers listed in Tables 1A-1B. In some embodiments, the transcriptomic signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80, 90, 100, or more of the genes of Tables 1A-1B. In some cases, the transcriptomic signature comprises genes 1-44 of Tables 1A-1B. In some cases, the transcriptomic signature comprises genes 1-117 of Tables 1A-1B. In some cases, the transcriptomic signature comprises one or more genes of Table 1A. In some cases, the transcriptomic signature comprises one or more genes of Table 1B. In some cases, the transcriptomic signature comprises or further comprises MIR155HG (or MIR155), the host gene for microRNA 155.

In some embodiments, the methods involve detecting in a biological sample from a subject the expression level of MIR155HG (or MIR155), the host gene for microRNA 155.

In some embodiments, gene expression profiling may be used as a research tool to identify new markers for diagnosis and/or classification of an IBD disease or condition, to monitor the effect of drugs or candidate drugs on biological samples and/or patients, to uncover new pathways for IBD treatment, or any combination thereof. In some embodiments, the treatment comprises a modulator of miR-155. In some embodiments, the treatment comprises a modulator of a kinase, such as a kinase of Table 20A. In some embodiments, the kinase modulator comprises an agent of Table 20B.

In some embodiments, the transcriptomic signature comprises ADAMTS1. In some embodiments, the transcriptomic signature comprises LCN2. In some embodiments, the transcriptomic signature comprises ADAM28. In some embodiments, the transcriptomic signature comprises TPSB2. In some embodiments, the transcriptomic signature comprises PPIAP30. In some embodiments, the transcriptomic signature comprises GFPT2. In some embodiments, the transcriptomic signature comprises KIT. In some embodiments, the transcriptomic signature comprises PLTP. In some embodiments, the transcriptomic signature comprises MFSD2A. In some embodiments, the transcriptomic signature comprises IL22. In some embodiments, the transcriptomic signature comprises LMCD1. In some embodiments, the transcriptomic signature comprises IL6. In some embodiments, the transcriptomic signature comprises TBC1D9. In some embodiments, the transcriptomic signature comprises CHAC1. In some embodiments, the transcriptomic signature comprises SEPP1. In some embodiments, the transcriptomic signature comprises SOD3. In some embodiments, the transcriptomic signature comprises RAB13. In some embodiments, the transcriptomic signature comprises LYZ. In some embodiments, the transcriptomic signature comprises CPA3. In some embodiments, the transcriptomic signature comprises SDS. In some embodiments, the transcriptomic signature comprises DYRK3. In some embodiments, the transcriptomic signature comprises DAB2. In some embodiments, the transcriptomic signature comprises TBC1D8. In some embodiments, the transcriptomic signature comprises CRYAB. In some embodiments, the transcriptomic signature comprises TBC1D3. In some embodiments, the transcriptomic signature comprises LRRC32. In some embodiments, the transcriptomic signature comprises SERPING1. In some embodiments, the transcriptomic signature comprises UBD. In some embodiments, the transcriptomic signature comprises FABP1. In some embodiments, the transcriptomic signature comprises SYK. In some embodiments, the transcriptomic signature comprises ALDOB. In some embodiments, the transcriptomic signature comprises SEMA6B. In some embodiments, the transcriptomic signature comprises NANOGNB. In some embodiments, the transcriptomic signature comprises DSE. In some embodiments, the transcriptomic signature comprises FPR3. In some embodiments, the transcriptomic signature comprises TNXB. In some embodiments, the transcriptomic signature comprises OR4A5. In some embodiments, the transcriptomic signature comprises DCN. In some embodiments, the transcriptomic signature comprises CHST15. In some embodiments, the transcriptomic signature comprises ADAMDEC1. In some embodiments, the transcriptomic signature comprises HDC. In some embodiments, the transcriptomic signature comprises RRAD. In some embodiments, the transcriptomic signature comprises C1S. In some embodiments, the transcriptomic signature comprises PLA2G2A. In some embodiments, the transcriptomic signature comprises CYCSP52. In some embodiments, the transcriptomic signature comprises C11orf96. In some embodiments, the transcriptomic signature comprises SEPSECS-AS1. In some embodiments, the transcriptomic signature comprises C1QC. In some embodiments, the transcriptomic signature comprises SLC9B1. In some embodiments, the transcriptomic signature comprises MLLT10P1. In some embodiments, the transcriptomic signature comprises LOC102724034. In some embodiments, the transcriptomic signature comprises SMOX. In some embodiments, the transcriptomic signature comprises CKB. In some embodiments, the transcriptomic signature comprises NCOR1P1. In some embodiments, the transcriptomic signature comprises LOC646736. In some embodiments, the transcriptomic signature comprises CLEC3B. In some embodiments, the transcriptomic signature comprises SLCO4A1. In some embodiments, the transcriptomic signature comprises APOC1P1. In some embodiments, the transcriptomic signature comprises KGFLP2. In some embodiments, the transcriptomic signature comprises ABI3BP. In some embodiments, the transcriptomic signature comprises LINC01189. In some embodiments, the transcriptomic signature comprises SEPT14. In some embodiments, the transcriptomic signature comprises FSTL1. In some embodiments, the transcriptomic signature comprises GEM. In some embodiments, the transcriptomic signature comprises FAM27A. In some embodiments, the transcriptomic signature comprises PTENP1-AS. In some embodiments, the transcriptomic signature comprises LIMS3L. In some embodiments, the transcriptomic signature comprises ST13P4. In some embodiments, the transcriptomic signature comprises C1QB. In some embodiments, the transcriptomic signature comprises HNRNPA1P33. In some embodiments, the transcriptomic signature comprises MIR663A. In some embodiments, the transcriptomic signature comprises LOC101927123. In some embodiments, the transcriptomic signature comprises C2orf27A. In some embodiments, the transcriptomic signature comprises LOC645166. In some embodiments, the transcriptomic signature comprises ZNF582-AS1. In some embodiments, the transcriptomic signature comprises HSPA2. In some embodiments, the transcriptomic signature comprises COL1A1. In some embodiments, the transcriptomic signature comprises COL5A1. In some embodiments, the transcriptomic signature comprises GOLGA6L5P. In some embodiments, the transcriptomic signature comprises PGM5-AS1. In some embodiments, the transcriptomic signature comprises CLDN10. In some embodiments, the transcriptomic signature comprises UBE2Q2L. In some embodiments, the transcriptomic signature comprises LOC100129138. In some embodiments, the transcriptomic signature comprises COL1A2. In some embodiments, the transcriptomic signature comprises SPARCL1. In some embodiments, the transcriptomic signature comprises FAM222A. In some embodiments, the transcriptomic signature comprises LINC00857. In some embodiments, the transcriptomic signature comprises CLIC4. In some embodiments, the transcriptomic signature comprises FAM182B. In some embodiments, the transcriptomic signature comprises LOC642426. In some embodiments, the transcriptomic signature comprises GYPE. In some embodiments, the transcriptomic signature comprises C8orf4. In some embodiments, the transcriptomic signature comprises RPSAP9. In some embodiments, the transcriptomic signature comprises FAM231A. In some embodiments, the transcriptomic signature comprises LINC00700. In some embodiments, the transcriptomic signature comprises ANKRD20A3. In some embodiments, the transcriptomic signature comprises FAM138D. In some embodiments, the transcriptomic signature comprises KRT20. In some embodiments, the transcriptomic signature comprises UBTFL1. In some embodiments, the transcriptomic signature comprises GAS7. In some embodiments, the transcriptomic signature comprises GPNMB. In some embodiments, the transcriptomic signature comprises TCF4. In some embodiments, the transcriptomic signature comprises LINC00348. In some embodiments, the transcriptomic signature comprises SRC. In some embodiments, the transcriptomic signature comprises HSPB6. In some embodiments, the transcriptomic signature comprises LOC100507006. In some embodiments, the transcriptomic signature comprises TCF21. In some embodiments, the transcriptomic signature comprises TMEM45B. In some embodiments, the transcriptomic signature comprises LOC101927905. In some embodiments, the transcriptomic signature comprises CXCL13. In some embodiments, the transcriptomic signature comprises AQP7P3. In some embodiments, the transcriptomic signature comprises PMP22. In some embodiments, the transcriptomic signature comprises LOC101928163. In some embodiments, the transcriptomic signature comprises REG3A. In some embodiments, the transcriptomic signature comprises MMP19. In some embodiments, the transcriptomic signature comprises PHLDB1. In some embodiments, the transcriptomic signature comprises LOC100508046. In some embodiments, the transcriptomic signature comprises SPINK4. In some embodiments, the transcriptomic signature comprises HES4. In some embodiments, the transcriptomic signature comprises TREM1. In some embodiments, the transcriptomic signature comprises TNFRSF12A. In some embodiments, the transcriptomic signature comprises PRKX-AS1. In some embodiments, the transcriptomic signature comprises PLGLB1. In some embodiments, the transcriptomic signature comprises SNAI1. In some embodiments, the transcriptomic signature comprises NUCB1-AS1. In some embodiments, the transcriptomic signature comprises BASP1. In some embodiments, the transcriptomic signature comprises MGP. In some embodiments, the transcriptomic signature comprises ANPEP. In some embodiments, the transcriptomic signature comprises PHACTR1. In some embodiments, the transcriptomic signature comprises ADM. In some embodiments, the transcriptomic signature comprises DEFA6. In some embodiments, the transcriptomic signature comprises VEGFA. In some embodiments, the transcriptomic signature comprises EGR2. In some embodiments, the transcriptomic signature comprises DEFA5. In some embodiments, the transcriptomic signature comprises CXCL3. In some embodiments, the transcriptomic signature comprises SDC4. In some embodiments, the transcriptomic signature comprises TPSAB1. In some embodiments, the transcriptomic signature comprises CD68. In some embodiments, the transcriptomic signature comprises EPAS1. In some embodiments, the transcriptomic signature comprises MARCKS. In some embodiments, the transcriptomic signature comprises TNFAIP2. In some embodiments, the transcriptomic signature comprises MIR663B. In some embodiments, the transcriptomic signature comprises TMEM114. In some embodiments, the transcriptomic signature comprises SIRPA. In some embodiments, the transcriptomic signature comprises GAS6. In some embodiments, the transcriptomic signature comprises IGFBP7. In some embodiments, the transcriptomic signature comprises ASB2. In some embodiments, the transcriptomic signature comprises HES1. In some embodiments, the transcriptomic signature comprises LOC284801. In some embodiments, the transcriptomic signature comprises TNFRSF13B. In some embodiments, the transcriptomic signature comprises MIR548I1. In some embodiments, the transcriptomic signature comprises DERL3. In some embodiments, the transcriptomic signature comprises SPARC. In some embodiments, the transcriptomic signature comprises EMP1. In some embodiments, the transcriptomic signature comprises LOC100240735. In some embodiments, the transcriptomic signature comprises LOC101927817. In some embodiments, the transcriptomic signature comprises STAB 1. In some embodiments, the transcriptomic signature comprises UPK3B. In some embodiments, the transcriptomic signature comprises RAB20. In some embodiments, the transcriptomic signature comprises MMP9. In some embodiments, the transcriptomic signature comprises MT1G. In some embodiments, the transcriptomic signature comprises POC1B-GALNT4. In some embodiments, the transcriptomic signature comprises CSF2RB. In some embodiments, the transcriptomic signature comprises IL1RN. In some embodiments, the transcriptomic signature comprises PLEKHA4. In some embodiments, the transcriptomic signature comprises LOC644172. In some embodiments, the transcriptomic signature comprises MAFF. In some embodiments, the transcriptomic signature comprises FDCSP. In some embodiments, the transcriptomic signature comprises DNASE1L3. In some embodiments, the transcriptomic signature comprises PTGS2. In some embodiments, the transcriptomic signature comprises TUBB6. In some embodiments, the transcriptomic signature comprises LINC01194. In some embodiments, the transcriptomic signature comprises CTAGE8. In some embodiments, the transcriptomic signature comprises REG1A. In some embodiments, the transcriptomic signature comprises ATP5J2-PTCD1. In some embodiments, the transcriptomic signature comprises DOK3. In some embodiments, the transcriptomic signature comprises EGR3. In some embodiments, the transcriptomic signature comprises AOAH-IT1. In some embodiments, the transcriptomic signature comprises RNASE1. In some embodiments, the transcriptomic signature comprises CCL11. In some embodiments, the transcriptomic signature comprises OR4F21. In some embodiments, the transcriptomic signature comprises FAM157B. In some embodiments, the transcriptomic signature comprises GATA2. In some embodiments, the transcriptomic signature comprises CTGF. In some embodiments, the transcriptomic signature comprises CXCL1. In some embodiments, the transcriptomic signature comprises GPX3. In some embodiments, the transcriptomic signature comprises FAM138A. In some embodiments, the transcriptomic signature comprises FAM138F. In some embodiments, the transcriptomic signature comprises FOSL1. In some embodiments, the transcriptomic signature comprises FSCN1. In some embodiments, the transcriptomic signature comprises FTH1P3. In some embodiments, the transcriptomic signature comprises SPHK1. In some embodiments, the transcriptomic signature comprises LOC441242. In some embodiments, the transcriptomic signature comprises UGT2B10. In some embodiments, the transcriptomic signature comprises MCTP1. In some embodiments, the transcriptomic signature comprises IL21R-AS1. In some embodiments, the transcriptomic signature comprises LOC285740. In some embodiments, the transcriptomic signature comprises HLA-L. In some embodiments, the transcriptomic signature comprises NPIPB9. In some embodiments, the transcriptomic signature comprises SEPT10. In some embodiments, the transcriptomics signature comprises miR-155. In some embodiments, the transcriptomic signature comprises ADH4. In some embodiments, the transcriptomic signature comprises ALG1L. In some embodiments, the transcriptomic signature comprises BCDIN3D. In some embodiments, the transcriptomic signature comprises C1orf106. In some embodiments, the transcriptomic signature comprises C2. In some embodiments, the transcriptomic signature comprises CCDC144NL. In some embodiments, the transcriptomic signature comprises CEACAM5. In some embodiments, the transcriptomic signature comprises CTAGE8. In some embodiments, the transcriptomic signature comprises DDX11L2. In some embodiments, the transcriptomic signature comprises DPPA4. In some embodiments, the transcriptomic signature comprises DUSP19. In some embodiments, the transcriptomic signature comprises FGB. In some embodiments, the transcriptomic signature comprises GP2. In some embodiments, the transcriptomic signature comprises GYPE. In some embodiments, the transcriptomic signature comprises HSD3B7. In some embodiments, the transcriptomic signature comprises HUNK. In some embodiments, the transcriptomic signature comprises JAM2. In some embodiments, the transcriptomic signature comprises KCNE3. In some embodiments, the transcriptomic signature comprises KRT42P. In some embodiments, the transcriptomic signature comprises LYZ. In some embodiments, the transcriptomic signature comprises MLLT10P1. In some embodiments, the transcriptomic signature comprises NAP1L6. In some embodiments, the transcriptomic signature comprises NEURL3. In some embodiments, the transcriptomic signature comprises NPIPB9. In some embodiments, the transcriptomic signature comprises PANK1. In some embodiments, the transcriptomic signature comprises PKIB. In some embodiments, the transcriptomic signature comprises RHOU. In some embodiments, the transcriptomic signature comprises RPSAP9. In some embodiments, the transcriptomic signature comprises SHCBP1. In some embodiments, the transcriptomic signature comprises SIGLEC8. In some embodiments, the transcriptomic signature comprises SLC15A2. In some embodiments, the transcriptomic signature comprises SLC25A34. In some embodiments, the transcriptomic signature comprises SLC6A20. In some embodiments, the transcriptomic signature comprises SLC9B1. In some embodiments, the transcriptomic signature comprises SYNPO2L. In some embodiments, the transcriptomic signature comprises TDGF1. In some embodiments, the transcriptomic signature comprises ZNF491. In some embodiments, the transcriptomic signature comprises ZNF620. In some embodiments, the transcriptomic signature comprises ZNF69. In some embodiments, the transcriptomic signature comprises CXCL16. In some embodiments, the transcriptomic signature comprises CD68. In some embodiments, the transcriptomic signature comprises CD300E.

The expression profile of a transcriptomic signature in a subject may be determined by analyzing genetic material obtained from a subject. The subject may be human. In some embodiments, the genetic material is obtained from a subject having an inflammatory disease, such as inflammatory bowel disease, or specifically, Crohn's Disease. Although the methods described herein are generally referenced for use with Crohn's Disease patients, in some cases the methods and transcriptomic signatures are applicable to other inflammatory diseases, including, ulcerative colitis.

In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., from the intestinal track of the subject. Isolating components of fluid or tissue samples (e.g., cells or RNA or DNA) may be accomplished using a variety of techniques. After the sample is obtained, it may be further processed to enrich for or purify genomic material.

In some embodiments, the expression level of a biomarker in a sample from a subject is compared to a reference expression level. In some cases, the reference expression level is from a subject that does not comprise IBD. In some cases, the reference expression level is from a subject that comprises a non-PBmu subtype of CD. In some cases, the reference expression level is from a subject that comprises a CD-PBmu subtype. In some cases, a patient having a CD-PBmu subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who does not have IBD or has a non-PBmu CD subtype). Table 2 provides non-limiting examples of increased expression fold of biomarkers in a CD-PBmu subject as compared to a subject who does not have IBD (NL) or has a PBT CD subtype. As used herein, Table 2 is inclusive of Table 2A and Table 2B.

TABLE 2A
Increased Expression of Biomarkers in CD-PBmu Subject

| | | Fold-change | Fold change |
|---|---|---|---|
| No. | Biomarker | PBmu vs PBT | PBmu vs NL |
| 1 | ADAM28 | 2.43 | |
| 2 | ADAMDEC1 | 6.76 | 4.658 |
| 3 | ADAMTS1 | 2.22 | 2.273 |
| 4 | ALDOB | 5.32 | 5.686 |
| 5 | C1S | 5.42 | 2.923 |
| 6 | CHAC1 | 4.65 | 3.857 |
| 7 | CHST15 | 3.18 | 2.211 |
| 8 | CPA3 | 5.19 | 5.849 |
| 9 | CRYAB | 6.32 | 5.2 |
| 10 | DAB2 | 2.29 | |
| 11 | DCN | 8.23 | 7.66 |
| 12 | DSE | 2.01 | 2.04 |
| 13 | DYRK3 | 3.79 | 3.357 |
| 14 | FABP1 | 6.38 | 3.571 |
| 15 | FPR3 | 4.35 | 4.133 |
| 16 | GFPT2 | 2.69 | |
| 17 | HDC | 5.99 | 5.357 |
| 18 | IL22 | 4.37 | |
| 19 | IL6 | 4.78 | 4.756 |
| 20 | KIT | 2.36 | 2.167 |
| 21 | LCN2 | 4.56 | |
| 22 | LMCD1 | 3.12 | 2.636 |
| 23 | LRRC32 | 2.83 | 2.267 |
| 24 | LYZ | 2.07 | 1.842 |
| 25 | MFSD2A | 3.13 | 2.611 |
| 26 | NANOGNB | 5.73 | 5.22 |
| 27 | OR4A5 | 11.69 | 6.429 |
| 28 | PLA2G2A | 7.93 | 3.429 |
| 29 | PLTP | 2.51 | |
| 30 | PPIAP30 | 3.01 | 3.258 |
| 31 | RAB13 | 2.09 | 1.787 |
| 32 | RRAD | 6.91 | 3.425 |
| 33 | SDS | 3.87 | 5 |
| 34 | SEMA6B | 5.8 | 3.714 |
| 35 | SEPP1 | 2.84 | 2.333 |
| 36 | SERPING1 | 4.12 | 4.343 |
| 37 | SOD3 | 5.11 | 3.929 |
| 38 | SYK | 2.34 | 1.761 |
| 39 | TBC1D3 | 11.52 | 5.867 |
| 40 | TBC1D8 | 2.01 | 2 |
| 41 | TBC1D9 | 2.26 | 1.859 |
| 42 | TNXB | 2.79 | 2.295 |
| 43 | TPSB2 | 4.12 | 3.5 |
| 44 | UBD | 6.82 | 4 |
| 45 | ABI3BP | 2.54 | 3.818 |
| 46 | ANKRD20A3 | 5.07 | 4.409 |
| 47 | APOC1P1 | 3.24 | 4.442 |
| 48 | AQP7P3 | 16.67 | 13.553 |
| 49 | C11orf96 | 3.99 | 3.621 |
| 50 | C1QB | 4.66 | 3.71 |
| 51 | C1QC | 4.19 | 6.14 |
| 52 | C2orf27A | 4.86 | 3.095 |
| 53 | C8orf4 | 8.42 | 6.176 |
| 54 | CKB | 3.13 | 1.867 |
| 55 | CLDN10 | 2.86 | 2.873 |
| 56 | CLEC3B | 4.85 | 2.55 |
| 57 | CLIC4 | 2.19 | 1.714 |
| 58 | COL1A1 | 5.31 | 4.028 |
| 59 | COL1A2 | 5.99 | 5.172 |
| 60 | COL5A1 | 2.69 | 2.529 |
| 61 | CXCL13 | 8.3 | 6.038 |
| 62 | CYCSP52 | 3.89 | 3.6 |
| 63 | FAM138D | 3.64 | 3.281 |
| 64 | FAM182B | 14.52 | 10.833 |
| 65 | FAM222A | 2.67 | 1.725 |
| 66 | FAM231A | 2.11 | 1.842 |
| 67 | FAM27A | 9.15 | 4.829 |
| 68 | FSTL1 | 4.4 | 4.824 |
| 69 | GAS7 | 2.21 | 1.591 |
| 70 | GEM | 4.97 | 5.542 |
| 71 | GOLGA6L5P | 3.44 | 2.067 |
| 72 | GPNMB | 6.33 | 4.59 |
| 73 | GYPE | 4.27 | 4.963 |
| 74 | HNRNPA1P33 | 7.75 | 3.278 |
| 75 | HSPA2 | 3.24 | 3.222 |
| 76 | HSPB6 | 6.69 | 4.386 |
| 77 | KGFLP2 | 2.6 | 2.083 |
| 78 | KRT20 | 7.48 | 5 |
| 79 | LIMS3L | 2.07 | 2 |
| 80 | LINC00348 | 3.85 | 2.932 |
| 81 | LINC00700 | 3.68 | 2.879 |
| 82 | LINC00857 | 2.26 | 1.907 |
| 83 | LINC01189 | 6.85 | 5.931 |
| 84 | LOC100129138 | 3.63 | 3.73 |
| 85 | LOC100507006 | 2.14 | 1.372 |
| 86 | LOC100508046 | 16.42 | 12.727 |
| 87 | LOC101927123 | 6.44 | |
| 88 | LOC101927905 | 3.39 | 2.864 |
| 89 | LOC101928163 | 5.48 | 4.151 |
| 90 | LOC102724034 | 2.85 | 1.8 |
| 91 | LOC642426 | 8.09 | 8.542 |
| 92 | LOC645166 | 4.71 | 6.258 |
| 93 | LOC646736 | 3.18 | 4.136 |
| 94 | MIR663A | 24.45 | 17.565 |
| 95 | MLLT10P1 | 2.37 | 3.687 |
| 96 | MMP19 | 7.06 | 4.066 |
| 97 | NCOR1P1 | 7.48 | 10.482 |
| 98 | PGM5-AS1 | 13.24 | 10.532 |
| 99 | PHLDB1 | 2.95 | 2.286 |
| 100 | PMP22 | 7.75 | 3.793 |
| 101 | PTENP1-AS | 5.08 | 5.882 |
| 102 | REG3A | 9.48 | 5.172 |
| 103 | RPSAP9 | 4.16 | 3.734 |
| 104 | SEPSECS-AS1 | 2.6 | |
| 105 | SEPT14 | 4.4 | |
| 106 | SLC9B1 | 2.43 | 2.175 |
| 107 | SLCO4A1 | 2.78 | 2.684 |
| 108 | SMOX | 3.01 | 2.229 |
| 109 | SPARCL1 | 5.83 | 4.561 |
| 110 | SRC | 2.42 | 2.418 |
| 111 | ST13P4 | 5.79 | 5.857 |
| 112 | TCF21 | 8.89 | 8.125 |
| 113 | TCF4 | 2.67 | 2.5 |
| 114 | TMEM45B | 2.05 | 1.585 |
| 115 | UBE2Q2L | 3.7 | 2.33 |
| 116 | UBTFL1 | 16.01 | 9.495 |
| 117 | ZNF582-AS1 | 2.43 | 1.766 |
| 118 | ADM | 3.54 | 3.296 |
| 119 | ANPEP | 2.77 | 2.262 |
| 120 | AOAH-IT1 | 5.73 | 3.767 |
| 121 | ASB2 | 2.16 | 1.629 |
| 122 | ATP5J2-PTCD1 | 8.72 | 2.679 |
| 123 | BASP1 | 2.38 | 1.976 |
| 124 | CCL11 | 7.01 | 4.242 |
| 125 | CD68 | 2.07 | 1.656 |
| 126 | CSF2RB | 2.58 | 2.061 |
| 127 | CTAGE8 | 3.47 | 2.03 |
| 128 | CTGF | 6.8 | 6.25 |
| 129 | CXCL1 | 9.88 | 8.571 |
| 130 | CXCL3 | 4.64 | 5.6 |
| 131 | DEFA5 | 5.86 | 5.161 |
| 132 | DEFA6 | 5.05 | 3.667 |
| 133 | DERL3 | 2.1 | 2.054 |
| 134 | DNASE1L3 | 5.79 | 3.167 |
| 135 | DOK3 | 3.05 | 2.118 |
| 136 | EGR2 | 2.12 | 3.57 |
| 137 | EGR3 | 3.37 | 4.522 |
| 138 | EMP1 | 3.72 | 3.056 |
| 139 | EPAS1 | 2.26 | 2.611 |
| 140 | FAM138A | 5.18 | 3.225 |
| 141 | FAM138F | 5.18 | 3.225 |
| 142 | FAM157B | 3.21 | 4.365 |
| 143 | FDCSP | 5.69 | 3.333 |
| 144 | FOSL1 | 3.85 | 3.851 |
| 145 | FSCN1 | 2.65 | 2.902 |
| 146 | FTH1P3 | 3.3 | 2.75 |
| 147 | GAS6 | 2.24 | 2.315 |
| 148 | GATA2 | 3.44 | 3.667 |
| 149 | GPX3 | 2.01 | 1.92 |
| 150 | HES1 | 4.07 | 4.9 |
| 151 | HES4 | 2.62 | 3.667 |
| 152 | HLA-L | 2.06 | 2.014 |
| 153 | IGFBP7 | 2.98 | 2.068 |
| 154 | IL1RN | 2.99 | 2.598 |
| 155 | IL21R-AS1 | 2.27 | 2.828 |
| 156 | LINC01194 | 6.64 | 2.952 |
| 157 | LOC100240735 | 2.26 | |
| 158 | LOC101927817 | 2.05 | 2.297 |
| 159 | LOC284801 | 2.66 | 4.337 |
| 160 | LOC285740 | 2.22 | 2.321 |
| 161 | LOC441242 | 2.11 | 1.901 |
| 162 | LOC644172 | 9.36 | 7.364 |
| 163 | MAFF | 2.04 | 2.345 |
| 164 | MARCKS | 2.36 | 2.637 |
| 165 | MCTP1 | 2.43 | 2.116 |
| 166 | MGP | 2.66 | 2.081 |
| 167 | MIR548I1 | 6.27 | 5.586 |
| 168 | MIR663B | 15.79 | 30.76 |
| 169 | MMP9 | 5.7 | 4.091 |
| 170 | MT1G | 7.38 | |
| 171 | NPIPB9 | 2.9 | 3.075 |
| 172 | NUCB1-AS1 | 4.88 | 4.429 |
| 173 | OR4F21 | 12.9 | 8.358 |
| 174 | PHACTR1 | 2.26 | 2.211 |
| 175 | PLEKHA4 | 2.88 | 2.278 |
| 176 | PLGLB1 | 2.42 | 2.678 |
| 177 | POC1B-GALNT4 | 6.4 | 5.075 |
| 178 | PRKX-AS1 | 2.53 | 1.952 |
| 179 | PTGS2 | 3.37 | 5.259 |
| 180 | RAB20 | 2.32 | 2.349 |
| 181 | REG1A | 6.54 | 6.818 |
| 182 | RNASE1 | 7.97 | 3.263 |
| 183 | SDC4 | 2.02 | 2.281 |
| 184 | SEPT10 | 2.38 | |
| 185 | SIRPA | 2.57 | 1.9 |
| 186 | SNAI1 | 2.82 | 3.238 |
| 187 | SPARC | 2.61 | 2.013 |
| 188 | SPHK1 | 4.35 | 3.226 |
| 189 | SPINK4 | 4.27 | |
| 190 | STAB1 | 3.03 | 2.145 |
| 191 | TMEM114 | 5.7 | 2.976 |
| 192 | TNFAIP2 | 2.68 | 2.376 |
| 193 | TNFRSF12A | 3.31 | 4.062 |
| 194 | TNFRSF13B | 3.17 | 2.316 |
| 195 | TPSAB1 | 3.89 | 3.667 |
| 196 | TREM1 | 2.72 | |
| 197 | TUBB6 | 2.55 | 2.039 |
| 198 | UGT2B10 | 11.04 | 10.69 |
| 199 | UPK3B | 2.08 | |
| 200 | VEGFA | 2.58 | 2.531 |

TABLE 2B
Increased Expression of Biomarkers in CD-PBmu Subject

| | PBmu | PBmu | | PBmu | PBmu |
|---|---|---|---|---|---|
| | vs PBT | vs NL | | vs PBT | vs NL |
| | Fold- | Fold- | | Fold- | Fold- |
| Biomarker | change | change | Biomarker | change | change |
| ADH4 | 2.18 | 2.4 | NAP1L6 | 2.31 | 2.26 |
| ALG1L | 2.9 | 2.09 | NEURL3 | 2.44 | 2.13 |
| BCDIN3D | 2.01 | 1.61 | NPIPB9 | 2.02 | 2.41 |
| C1orf106 | 2.15 | 2.2 | PANK1 | 2.13 | 1.88 |
| C2 | 2.22 | 1.85 | PKIB | 2.35 | 3.56 |
| CCDC144NL | 2.48 | 3.71 | RHOU | 2.34 | 2.2 |
| CEACAM5 | 2.26 | 2.84 | RPSAP9 | 2.61 | 1.68 |
| CTAGE8 | 2.02 | 1.73 | SHCBP1 | 2.14 | 2.1 |
| DDX11L2 | 2.31 | 2.14 | SIGLEC8 | 2.22 | 1.56 |
| DPPA4 | 2.25 | 1.31 | SLC15A2 | 2.05 | 2.41 |
| DUSP19 | 2.12 | 2.32 | SLC25A34 | 2.14 | 1.72 |
| FGB | 2.34 | 1.6 | SLC6A20 | 2.31 | 2.35 |
| GP2 | 2.53 | 1.92 | SLC9B1 | 2.29 | 1.54 |
| GYPE | 2.29 | 1.54 | SYNPO2L | 2.38 | 1.72 |
| HSD3B7 | 2.24 | 2.2 | TDGF1 | 2.16 | 1.8 |
| HUNK | 2.2 | 2.28 | ZNF491 | 2.07 | 1.34 |
| JAM2 | 2.35 | 2.74 | ZNF620 | 2.14 | 2.39 |
| KCNE3 | 2.3 | 1.59 | ZNF69 | 2.07 | 2.15 |
| KRT42P | 2.02 | 2.05 | CXCL16 | 2.15 | 1.66 |
| LYZ | 2.61 | 1.89 | CD68 | 2.13 | 1.7 |
| MLLT10P1 | 2.52 | 1.78 | CD300E | 2.91 | 1.61 |

In embodiments where more than one biomarker is detected, the differences in expression between a patient having a CD-PBmu subtype and a reference subject (e.g., non-IBD subject or subject with CD PBT) may be different for each marker, e.g., each of the biomarkers detected is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference non-CD-PBmu sample. In some cases, at least about 80%, 85%, 90%, 95%, 96%, 97%, 98%, or 99% of the biomarkers detected in a transcriptomic signature is at least about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 4, about 5, about 6, about 7, about 8, about 9, about 10, or about 15 fold up-modulated as compared to the expression level of the respective biomarker in the reference non-CD-PBmu sample.

### Monocyte Signature and Profiling

In one aspect, provided herein are monocyte signatures associated with a subtype of IBD, including CD. In some cases, the monocyte signature comprises one or more genes of Table 17A. In some cases, the monocyte signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 17A.

Further provided are methods and compositions for characterizing a subtype of Crohn's Disease (CD) in a subject. Non-limiting examples of subtypes are monocyte 2 subtype and monocyte 1 subtype. The characterization methods provided include diagnosing the presence or absence of a CD subtype, prognosing whether a subject is predisposed to developing a particular CD subtype, prognosing a response of a patient with a particular CD subtype to a therapeutic treatment, and monitoring CD treatment. In some embodiments, the treatment comprises a modulator of miR-155. In some embodiments, the treatment comprises a modulator of a kinase, such as a kinase of Table 20A. In some embodiments, the kinase modulator comprises an agent of Table 20B.

In some embodiments, the methods involve detecting in a biological sample comprising monocytes from a subject expression levels of one or more genes of a monocyte signature to obtain an expression profile comprising the expression levels of each of the one or more genes in the signature. In some embodiments, the monocyte signature comprises one or more biomarkers listed in Table 17A. In some embodiments, the monocyte signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 17A.

The expression profile of a monocyte signature in a subject may be determined by analyzing monocytes of a subject. The subject may be human. In some embodiments, the monocytes are obtained from a subject having an inflammatory disease, such as inflammatory bowel disease, or specifically, Crohn's Disease. Although the methods described herein are generally referenced for use with Crohn's Disease patients, in some cases the methods and monocyte signatures are applicable to other inflammatory diseases, including, ulcerative colitis.

In some embodiments, the expression level of a biomarker in a sample from a subject is compared to a reference expression level. In some cases, the reference expression level is from a subject that does not comprise IBD. In some cases, the reference expression level is from a subject that comprises a monocyte 1 subtype of CD. In some cases, the reference expression level is from a subject that comprises a monocyte 2 subtype of CD. In some cases, a patient having a monocyte 2 subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who has a monocyte 1 subtype). In some cases, a patient having a monocyte 1 subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who has a monocyte 2 subtype). Table 17A provides non-limiting examples of expression fold of biomarkers in a monocyte 1 subtype as compared to a monocyte 2 subtype.

### Expression and RNA Sequencing Methods

Any suitable method can be utilized to assess (directly or indirectly) the level of expression of a biomarker in a sample. Non-limiting examples of such methods include analyzing the sample using nucleic acid hybridization methods, nucleic acid reverse transcription methods, nucleic acid amplification methods, array analysis, and combinations thereof. In some embodiments, the level of expression of a biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for RNA sample analysis include microarray analysis (e.g., using Affymetrix's microarray system or Illumina's BeadArray Technology).

Isolated RNA can be used in hybridization or amplification assays that include, but are not limited to, Southern or Northern analyses, polymerase chain reaction (PCR) analyses and probe arrays. An exemplary method for the determination of RNA levels involves contacting RNA with a nucleic acid molecule (e.g., probe) that can hybridize to the biomarker mRNA. The nucleic acid molecule can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the biomarker genomic DNA. In some embodiments, the RNA is immobilized on a solid surface and contacted with a probe, for example by running the isolated RNA on an agarose gel and transferring the RNA from the gel to a membrane, such as nitrocellulose. In some embodiments, the probe(s) are immobilized on a solid surface, for example, in an Affymetrix gene chip array, and the probe(s) are contacted with RNA.

The level of expression of the biomarker in a sample can also be determined using methods that involve the use of nucleic acid amplification and/or reverse transcriptase, e.g., by RT-PCR, ligase chain reaction, self-sustained sequence replication, transcriptional amplification system, Q-Beta Replicase, rolling circle replication or any other nucleic acid amplification method, followed by the detection of the amplified molecules. These approaches may be useful for the detection of nucleic acid molecules if such molecules are present in very low numbers. In some embodiments, the level of expression of the biomarker is determined by quantitative fluorogenic RT-PCR (e.g., the TaqMan™ System). Such methods may utilize pairs of oligonucleotide primers that are specific for the biomarker.

In some embodiments, biomarker expression is determined by sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

The expression levels of biomarker RNA can be monitored using a membrane blot (such as used in hybridization analysis such as Northern, Southern, dot, and the like), microwells, sample tubes, gels, beads, fibers, or any solid support comprising bound nucleic acids. The determination of biomarker expression level may also comprise using nucleic acid probes in solution.

In some embodiments, microarrays are used to detect the level of expression of a biomarker. DNA microarrays provide one method for the simultaneous measurement of the expression levels of large numbers of genes. Each array consists of a reproducible pattern of capture probes attached to a solid support. Labeled nucleic acid is hybridized to complementary probes on the array and then detected, e.g., by laser scanning Hybridization intensities for each probe on the array are determined and converted to a quantitative value representing relative gene expression levels. High-density oligonucleotide arrays may be useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some instances, a method of detecting an expression profile in a subject comprises contacting nucleic acids from a sample of the subject with a nucleic acid polymer that hybridizes to a region of a biomarker nucleic acid sequence. Hybridization may occur at standard hybridization temperatures, e.g., between about 35° C. and about 65° C. in a standard PCR buffer. In some cases, the biomarker nucleic acid sequence is a sequence comprising at least about 30, 40, 50, 60, 70, 80, 90, or 100 nucleobases of a biomarker listed in Tables 1A-1B, Table 16, or Table 17A. The nucleic acid polymer can comprise an oligonucleotide of at least or about 5, 10, 15, 20, 25, 30, 35, 40, 45, 50, 75, 100 or more nucleobases in length and sufficient to specifically hybridize to a biomarker of Tables 1A-1B, Table 16, or Table 17A. In some instances, the nucleic acid polymer comprises between about 10 and about 100 nucleobases, between about 10 and about 75 nucleobases, between about 10 and about 50 nucleobases, between about 15 and about 100 nucleobases, between about 15 and about 75 nucleobases, between about 15 and about 50 nucleobases, between about 20 and about 100 nucleobases, between about 20 and about 75 nucleobases, between about 20 and about 50 nucleobases, between about 25 and about 100 nucleobases, between about 25 and about 75 nucleobases, or between about 25 and about 50 nucleobases.

Provided herein is a nucleic acid polymer that specifically hybridizes to ADAMTS1. Provided herein is a nucleic acid polymer that specifically hybridizes to LCN2. Provided herein is a nucleic acid polymer that specifically hybridizes to ADAM28. Provided herein is a nucleic acid polymer that specifically hybridizes to TPSB2. Provided herein is a nucleic acid polymer that specifically hybridizes to PPIAP30. Provided herein is a nucleic acid polymer that specifically hybridizes to GFPT2. Provided herein is a nucleic acid polymer that specifically hybridizes to KIT. Provided herein is a nucleic acid polymer that specifically hybridizes to PLTP. Provided herein is a nucleic acid polymer that specifically hybridizes to MFSD2A. Provided herein is a nucleic acid polymer that specifically hybridizes to IL22. Provided herein is a nucleic acid polymer that specifically hybridizes to LMCD1. Provided herein is a nucleic acid polymer that specifically hybridizes to IL6. Provided herein is a nucleic acid polymer that specifically hybridizes to TBC1D9. Provided herein is a nucleic acid polymer that specifically hybridizes to CHAC1. Provided herein is a nucleic acid polymer that specifically hybridizes to SEPP1. Provided herein is a nucleic acid polymer that specifically hybridizes to SOD3. Provided herein is a nucleic acid polymer that specifically hybridizes to RAB13. Provided herein is a nucleic acid polymer that specifically hybridizes to LYZ. Provided herein is a nucleic acid polymer that specifically hybridizes to CPA3. Provided herein is a nucleic acid polymer that specifically hybridizes to SDS. Provided herein is a nucleic acid polymer that specifically hybridizes to DYRK3. Provided herein is a nucleic acid polymer that specifically hybridizes to DAB2. Provided herein is a nucleic acid polymer that specifically hybridizes to TBC1D8. Provided herein is a nucleic acid polymer that specifically hybridizes to CRYAB. Provided herein is a nucleic acid polymer that specifically hybridizes to TBC1D3. Provided herein is a nucleic acid polymer that specifically hybridizes to LRRC32. Provided herein is a nucleic acid polymer that specifically hybridizes to SERPING1. Provided herein is a nucleic acid polymer that specifically hybridizes to UBD. Provided herein is a nucleic acid polymer that specifically hybridizes to FABP1. Provided herein is a nucleic acid polymer that specifically hybridizes to SYK. Provided herein is a nucleic acid polymer that specifically hybridizes to ALDOB. Provided herein is a nucleic acid polymer that specifically hybridizes to SEMA6B. Provided herein is a nucleic acid polymer that specifically hybridizes to NANOGNB. Provided herein is a nucleic acid polymer that specifically hybridizes to DSE. Provided herein is a nucleic acid polymer that specifically hybridizes to FPR3. Provided herein is a nucleic acid polymer that specifically hybridizes to TNXB. Provided herein is a nucleic acid polymer that specifically hybridizes to OR4A5. Provided herein is a nucleic acid polymer that specifically hybridizes to DCN. Provided herein is a nucleic acid polymer that specifically hybridizes to CHST15. Provided herein is a nucleic acid polymer that specifically hybridizes to ADAMDEC1. Provided herein is a nucleic acid polymer that specifically hybridizes to HDC. Provided herein is a nucleic acid polymer that specifically hybridizes to RRAD. Provided herein is a nucleic acid polymer that specifically hybridizes to C1S. Provided herein is a nucleic acid polymer that specifically hybridizes to PLA2G2A. Provided herein is a nucleic acid polymer that specifically hybridizes to CYCSP52. Provided herein is a nucleic acid polymer that specifically hybridizes to C11orf96. Provided herein is a nucleic acid polymer that specifically hybridizes to SEPSECS-AS1. Provided herein is a nucleic acid polymer that specifically hybridizes to C1QC. Provided herein is a nucleic acid polymer that specifically hybridizes to SLC9B1. Provided herein is a nucleic acid polymer that specifically hybridizes to MLLT10P1. Provided herein is a nucleic acid polymer that specifically hybridizes to LOC102724034. Provided herein is a nucleic acid polymer that specifically hybridizes to SMOX. Provided herein is a nucleic acid polymer that specifically hybridizes to CKB. Provided herein is a nucleic acid polymer that specifically hybridizes to NCOR1P1. Provided herein is a nucleic acid polymer that specifically hybridizes to LOC646736. Provided herein is a nucleic acid polymer that specifically hybridizes to CLEC3B. Provided herein is a nucleic acid polymer that specifically hybridizes to SLCO4A1. Provided herein is a nucleic acid polymer that specifically hybridizes to APOC1P1. Provided herein is a nucleic acid polymer that specifically hybridizes to KGFLP2. Provided herein is a nucleic acid polymer that specifically hybridizes to ABI3BP. Provided herein is a nucleic acid polymer that specifically hybridizes to LINC01189. Provided herein is a nucleic acid polymer that specifically hybridizes to SEPT14. Provided herein is a nucleic acid polymer that specifically hybridizes to FSTL1. Provided herein is a nucleic acid polymer that specifically hybridizes to GEM. Provided herein is a nucleic acid polymer that specifically hybridizes to FAM27A. Provided herein is a nucleic acid polymer that specifically hybridizes to PTENP1-AS. Provided herein is a nucleic acid polymer that specifically hybridizes to LIMS3L. Provided herein is a nucleic acid polymer that specifically hybridizes to ST13P4. Provided herein is a nucleic acid polymer that specifically hybridizes to C1QB. Provided herein is a nucleic acid polymer that specifically hybridizes to HNRNPA1P33. Provided herein is a nucleic acid polymer that specifically hybridizes to MIR663A. Provided herein is a nucleic acid polymer that specifically hybridizes to LOC101927123. Provided herein is a nucleic acid polymer that specifically hybridizes to C2orf27A. Provided herein is a nucleic acid polymer that specifically hybridizes to LOC645166. Provided herein is a nucleic acid polymer that specifically hybridizes to ZNF582-AS1. Provided herein is a nucleic acid polymer that specifically hybridizes to HSPA2. Provided herein is a nucleic acid polymer that specifically hybridizes to COL1A1. Provided herein is a nucleic acid polymer that specifically hybridizes to COL5A1. Provided herein is a nucleic acid polymer that specifically hybridizes to GOLGA6L5P. Provided herein is a nucleic acid polymer that specifically hybridizes to PGM5-AS1. Provided herein is a nucleic acid polymer that specifically hybridizes to CLDN10. Provided herein is a nucleic acid polymer that specifically hybridizes to UBE2Q2L. Provided herein is a nucleic acid polymer that specifically hybridizes to LOC100129138. Provided herein is a nucleic acid polymer that specifically hybridizes to COL1A2. Provided herein is a nucleic acid polymer that specifically hybridizes to SPARCL1. Provided herein is a nucleic acid polymer that specifically hybridizes to FAM222A. Provided herein is a nucleic acid polymer that specifically hybridizes to LINC00857. Provided herein is a nucleic acid polymer that specifically hybridizes to CLIC4. Provided herein is a nucleic acid polymer that specifically hybridizes to FAM182B. Provided herein is a nucleic acid polymer that specifically hybridizes to LOC642426. Provided herein is a nucleic acid polymer that specifically hybridizes to GYPE. Provided herein is a nucleic acid polymer that specifically hybridizes to C8orf4. Provided herein is a nucleic acid polymer that specifically hybridizes to RPSAP9. Provided herein is a nucleic acid polymer that specifically hybridizes to FAM231A. Provided herein is a nucleic acid polymer that specifically hybridizes to LINC00700. Provided herein is a nucleic acid polymer that specifically hybridizes to ANKRD20A3. Provided herein is a nucleic acid polymer that specifically hybridizes to FAM138D. Provided herein is a nucleic acid polymer that specifically hybridizes to KRT20. Provided herein is a nucleic acid polymer that specifically hybridizes to UBTFL1. Provided herein is a nucleic acid polymer that specifically hybridizes to GAS7. Provided herein is a nucleic acid polymer that specifically hybridizes to GPNMB. Provided herein is a nucleic acid polymer that specifically hybridizes to TCF4. Provided herein is a nucleic acid polymer that specifically hybridizes to LINC00348. Provided herein is a nucleic acid polymer that specifically hybridizes to SRC. Provided herein is a nucleic acid polymer that specifically hybridizes to HSPB6. Provided herein is a nucleic acid polymer that specifically hybridizes to LOC100507006. Provided herein is a nucleic acid polymer that specifically hybridizes to TCF21. Provided herein is a nucleic acid polymer that specifically hybridizes to TMEM45B. Provided herein is a nucleic acid polymer that specifically hybridizes to LOC101927905. Provided herein is a nucleic acid polymer that specifically hybridizes to CXCL13. Provided herein is a nucleic acid polymer that specifically hybridizes to AQP7P3. Provided herein is a nucleic acid polymer that specifically hybridizes to PMP22. Provided herein is a nucleic acid polymer that specifically hybridizes to LOC101928163. Provided herein is a nucleic acid polymer that specifically hybridizes to REG3A. Provided herein is a nucleic acid polymer that specifically hybridizes to MMP19. Provided herein is a nucleic acid polymer that specifically hybridizes to PHLDB1. Provided herein is a nucleic acid polymer that specifically hybridizes to LOC100508046. Provided herein is a nucleic acid polymer that specifically hybridizes to SPINK4. Provided herein is a nucleic acid polymer that specifically hybridizes to HES4. Provided herein is a nucleic acid polymer that specifically hybridizes to TREM1. Provided herein is a nucleic acid polymer that specifically hybridizes to TNFRSF12A. Provided herein is a nucleic acid polymer that specifically hybridizes to PRKX-AS1. Provided herein is a nucleic acid polymer that specifically hybridizes to PLGLB1. Provided herein is a nucleic acid polymer that specifically hybridizes to SNAI1. Provided herein is a nucleic acid polymer that specifically hybridizes to NUCB1-AS1. Provided herein is a nucleic acid polymer that specifically hybridizes to BASP1. Provided herein is a nucleic acid polymer that specifically hybridizes to MGP. Provided herein is a nucleic acid polymer that specifically hybridizes to ANPEP. Provided herein is a nucleic acid polymer that specifically hybridizes to PHACTR1. Provided herein is a nucleic acid polymer that specifically hybridizes to ADM. Provided herein is a nucleic acid polymer that specifically hybridizes to DEFA6. Provided herein is a nucleic acid polymer that specifically hybridizes to VEGFA. Provided herein is a nucleic acid polymer that specifically hybridizes to EGR2. Provided herein is a nucleic acid polymer that specifically hybridizes to DEFA5. Provided herein is a nucleic acid polymer that specifically hybridizes to CXCL3. Provided herein is a nucleic acid polymer that specifically hybridizes to SDC4. Provided herein is a nucleic acid polymer that specifically hybridizes to TPSAB1. Provided herein is a nucleic acid polymer that specifically hybridizes to CD68. Provided herein is a nucleic acid polymer that specifically hybridizes to EPAS1. Provided herein is a nucleic acid polymer that specifically hybridizes to MARCKS. Provided herein is a nucleic acid polymer that specifically hybridizes to TNFAIP2. Provided herein is a nucleic acid polymer that specifically hybridizes to MIR663B. Provided herein is a nucleic acid polymer that specifically hybridizes to TMEM114. Provided herein is a nucleic acid polymer that specifically hybridizes to SIRPA. Provided herein is a nucleic acid polymer that specifically hybridizes to GAS6. Provided herein is a nucleic acid polymer that specifically hybridizes to IGFBP7. Provided herein is a nucleic acid polymer that specifically hybridizes to ASB2. Provided herein is a nucleic acid polymer that specifically hybridizes to HES1. Provided herein is a nucleic acid polymer that specifically hybridizes to LOC284801. Provided herein is a nucleic acid polymer that specifically hybridizes to TNFRSF13B. Provided herein is a nucleic acid polymer that specifically hybridizes to MIR548I1. Provided herein is a nucleic acid polymer that specifically hybridizes to DERL3. Provided herein is a nucleic acid polymer that specifically hybridizes to SPARC. Provided herein is a nucleic acid polymer that specifically hybridizes to EMP1. Provided herein is a nucleic acid polymer that specifically hybridizes to LOC100240735. Provided herein is a nucleic acid polymer that specifically hybridizes to LOC101927817. Provided herein is a nucleic acid polymer that specifically hybridizes to STAB 1. Provided herein is a nucleic acid polymer that specifically hybridizes to UPK3B. Provided herein is a nucleic acid polymer that specifically hybridizes to RAB20. Provided herein is a nucleic acid polymer that specifically hybridizes to MMP9. Provided herein is a nucleic acid polymer that specifically hybridizes to MT1G. Provided herein is a nucleic acid polymer that specifically hybridizes to POC1B-GALNT4. Provided herein is a nucleic acid polymer that specifically hybridizes to CSF2RB. Provided herein is a nucleic acid polymer that specifically hybridizes to IL1RN. Provided herein is a nucleic acid polymer that specifically hybridizes to PLEKHA4. Provided herein is a nucleic acid polymer that specifically hybridizes to LOC644172. Provided herein is a nucleic acid polymer that specifically hybridizes to MAFF. Provided herein is a nucleic acid polymer that specifically hybridizes to FDCSP. Provided herein is a nucleic acid polymer that specifically hybridizes to DNASE1L3. Provided herein is a nucleic acid polymer that specifically hybridizes to PTGS2. Provided herein is a nucleic acid polymer that specifically hybridizes to TUBB6. Provided herein is a nucleic acid polymer that specifically hybridizes to LINC01194. Provided herein is a nucleic acid polymer that specifically hybridizes to CTAGE8. Provided herein is a nucleic acid polymer that specifically hybridizes to REG1A. Provided herein is a nucleic acid polymer that specifically hybridizes to ATP5J2-PTCD1. Provided herein is a nucleic acid polymer that specifically hybridizes to DOK3. Provided herein is a nucleic acid polymer that specifically hybridizes to EGR3. Provided herein is a nucleic acid polymer that specifically hybridizes to AOAH-IT1. Provided herein is a nucleic acid polymer that specifically hybridizes to RNASE1. Provided herein is a nucleic acid polymer that specifically hybridizes to CCL11. Provided herein is a nucleic acid polymer that specifically hybridizes to OR4F21. Provided herein is a nucleic acid polymer that specifically hybridizes to FAM157B. Provided herein is a nucleic acid polymer that specifically hybridizes to GATA2. Provided herein is a nucleic acid polymer that specifically hybridizes to CTGF. Provided herein is a nucleic acid polymer that specifically hybridizes to CXCL1. Provided herein is a nucleic acid polymer that specifically hybridizes to GPX3. Provided herein is a nucleic acid polymer that specifically hybridizes to FAM138A. Provided herein is a nucleic acid polymer that specifically hybridizes to FAM138F. Provided herein is a nucleic acid polymer that specifically hybridizes to FOSL1. Provided herein is a nucleic acid polymer that specifically hybridizes to FSCN1. Provided herein is a nucleic acid polymer that specifically hybridizes to FTH1P3. Provided herein is a nucleic acid polymer that specifically hybridizes to SPHK1. Provided herein is a nucleic acid polymer that specifically hybridizes to LOC441242. Provided herein is a nucleic acid polymer that specifically hybridizes to UGT2B10. Provided herein is a nucleic acid polymer that specifically hybridizes to MCTP1. Provided herein is a nucleic acid polymer that specifically hybridizes to IL21R-AS1. Provided herein is a nucleic acid polymer that specifically hybridizes to LOC285740. Provided herein is a nucleic acid polymer that specifically hybridizes to HLA-L. Provided herein is a nucleic acid polymer that specifically hybridizes to NPIPB9. Provided herein is a nucleic acid polymer that specifically hybridizes to SEPT10. Provided herein is a nucleic acid polymer that specifically hybridizes to miR-155. Provided herein is a nucleic acid polymer that specifically hybridizes to ADH4. Provided herein is a nucleic acid polymer that specifically hybridizes to ALG1L. Provided herein is a nucleic acid polymer that specifically hybridizes to BCDIN3D. Provided herein is a nucleic acid polymer that specifically hybridizes to C1orf106. Provided herein is a nucleic acid polymer that specifically hybridizes to C2. Provided herein is a nucleic acid polymer that specifically hybridizes to CCDC144NL. Provided herein is a nucleic acid polymer that specifically hybridizes to CEACAM5. Provided herein is a nucleic acid polymer that specifically hybridizes to CTAGE8. Provided herein is a nucleic acid polymer that specifically hybridizes to DDX11L2. Provided herein is a nucleic acid polymer that specifically hybridizes to DPPA4. Provided herein is a nucleic acid polymer that specifically hybridizes to DUSP19. Provided herein is a nucleic acid polymer that specifically hybridizes to FGB. Provided herein is a nucleic acid polymer that specifically hybridizes to GP2. Provided herein is a nucleic acid polymer that specifically hybridizes to GYPE. Provided herein is a nucleic acid polymer that specifically hybridizes to HSD3B7. Provided herein is a nucleic acid polymer that specifically hybridizes to HUNK. Provided herein is a nucleic acid polymer that specifically hybridizes to JAM2. Provided herein is a nucleic acid polymer that specifically hybridizes to KCNE3. Provided herein is a nucleic acid polymer that specifically hybridizes to KRT42P. Provided herein is a nucleic acid polymer that specifically hybridizes to LYZ. Provided herein is a nucleic acid polymer that specifically hybridizes to MLLT10P1. Provided herein is a nucleic acid polymer that specifically hybridizes to NAP1L6. Provided herein is a nucleic acid polymer that specifically hybridizes to NEURL3. Provided herein is a nucleic acid polymer that specifically hybridizes to NPIPB9. Provided herein is a nucleic acid polymer that specifically hybridizes to PANK1. Provided herein is a nucleic acid polymer that specifically hybridizes to PKIB. Provided herein is a nucleic acid polymer that specifically hybridizes to RHOU. Provided herein is a nucleic acid polymer that specifically hybridizes to RPSAP9. Provided herein is a nucleic acid polymer that specifically hybridizes to SHCBP1. Provided herein is a nucleic acid polymer that specifically hybridizes to SIGLEC8. Provided herein is a nucleic acid polymer that specifically hybridizes to SLC15A2. Provided herein is a nucleic acid polymer that specifically hybridizes to SLC25A34. Provided herein is a nucleic acid polymer that specifically hybridizes to SLC6A20. Provided herein is a nucleic acid polymer that specifically hybridizes to SLC9B1. Provided herein is a nucleic acid polymer that specifically hybridizes to SYNPO2L. Provided herein is a nucleic acid polymer that specifically hybridizes to TDGF1. Provided herein is a nucleic acid polymer that specifically hybridizes to ZNF491. Provided herein is a nucleic acid polymer that specifically hybridizes to ZNF620. Provided herein is a nucleic acid polymer that specifically hybridizes to ZNF69. Provided herein is a nucleic acid polymer that specifically hybridizes to CXCL16. Provided herein is a nucleic acid polymer that specifically hybridizes to CD68. Provided herein is a nucleic acid polymer that specifically hybridizes to CD300E.

Nucleic acid polymers include primers useful for amplifying a nucleic acid of biomarker provided in Tables 1A-1B, Table 16, Table 17A, or Table 14. For example, for use in an amplification assay such as qPCR. Nucleic acid polymers also include probes comprising a detectable label for detecting and/or quantifying a biomarker of Tables 1A-1B, Table 16, Table 17A, or Table 14. In some cases, the probes are reporters that comprise a dye label on one end and a quencher on the other end. When the probes are hybridized to a biomarker nucleic acid, an added DNA polymerase may cleave those hybridized probes, separating the reporter dye from the quencher, and thus increasing fluorescence by the reporter. In some cases, provided is a probe comprising a nucleic acid polymer described herein.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-(2,5-dioxo-1-pyrrolidinyl)oxy]-6-oxohexyl]aminolsulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for hybridization to a biomarker of Tables 1A-1B, Table 16 or Table 17A are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein for determining an expression profile in a subject comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a biomarker nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a biomarker nucleic acid.

### Profile Analysis

The expression profile of a patient sample (test sample) may be compared to a reference sample, e.g., a sample from a subject who does not have IBD such as CD (normal sample), or a sample from a subject who has a non-CD-PBmu subtype. In some cases, a normal sample is that which is or is expected to be free of IBD disease or condition, or a sample that would test negative for any IBD disease or condition. The reference sample may be assayed at the same time, or at a different time from the test sample. In some cases, the expression profile of a reference sample is obtained and stored in a database for comparison to the test sample.

The results of an assay on the test sample may be compared to the results of the same assay on a reference sample. In some cases, the results of the assay on the normal sample are from a database. In some cases, the results of the assay on the normal sample are a known or generally accepted value by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, coy number variations, indications of the presence or absence of one or more DNA markers or regions, and/or nucleic acid sequences.

In some embodiments, the gene expression profile of a test sample is evaluated using methods for correlating gene product expression levels with a specific phenotype of CD, such as the CD-PBmu subtype described herein. In some cases, a specified statistical confidence level may be determined in order to provide a diagnostic confidence level. For example, it may be determined that a confidence level of greater than 90% may be a useful predictor of CD-PBmu. In other embodiments, more or less stringent confidence levels may be chosen. For example, a confidence level of approximately 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and the number of gene expression products analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operator Curve analysis (ROC), binormal ROC, principal component analysis, partial least squares analysis, singular value decomposition, least absolute shrinkage and selection operator analysis, least angle regression, and the threshold gradient directed regularization method.

Raw gene expression level data may in some cases be improved through the application of algorithms designed to normalize and or improve the reliability of the data. In some embodiments of the present invention the data analysis requires a computer or other device, machine or apparatus for application of the various algorithms described herein due to the large number of individual data points that are processed. A “machine learning algorithm” refers to a computational-based prediction methodology, also known as a “classifier”, employed for characterizing a gene expression profile. The signals corresponding to certain expression levels, which are obtained by, e.g., microarray-based hybridization assays or sequencing, are typically subjected to the algorithm in order to classify the expression profile. Supervised learning generally involves “training” a classifier to recognize the distinctions among classes and then “testing” the accuracy of the classifier on an independent test set. For test samples the classifier can be used to predict the class in which the samples belong.

In some cases, the robust multi-array Average (RMA) method may be used to normalize the raw data. The RMA method begins by computing background-corrected intensities for each matched cell on a number of microarrays. The background corrected values are restricted to positive values as described by Irizarry et al. Biostatistics 2003 Apr. 4 (2): 249-64. The back-ground corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Data may further be filtered to remove data that may be considered suspect. In some embodiments, data deriving from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some cases, unreliable probe sets may be selected for exclusion from data analysis by ranking probe-set reliability against a series of reference datasets. For example, RefSeq or Ensembl (EMBL) are considered very high-quality reference datasets. Data from probe sets matching RefSeq or Ensembl sequences may in some cases be specifically included in microarray analysis experiments due to their expected high reliability. Similarly, data from probe-sets matching less reliable reference datasets may be excluded from further analysis, or considered on a case by case basis for inclusion. In some cases, the Ensembl high throughput cDNA (HTC) and/or mRNA reference datasets may be used to determine the probe-set reliability separately or together. In other cases, probe-set reliability may be ranked. For example, probes and/or probe-sets that match perfectly to all reference datasets such as for example RefSeq, HTC, and mRNA, may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there are many methods understood in the art for assessing the reliability of a given probe and/or probe-set for molecular profiling and the methods of the present invention encompass any of these methods and combinations thereof.

The results of the expression profile may be analyzed to classify a subject as having or lacking an IBD disease or condition, such as a CD-PBmu subtype. In some cases, a diagnostic result may indicate a certain molecular pathway involved in the IBD disease or condition, or a certain grade or stage of a particular IBD disease or condition. In some cases, a diagnostic result may inform an appropriate therapeutic intervention, such as a specific drug regimen like a molecule that targets a biomolecule in a pathway of any biomarker in Tables 1A-1B, 16, or 17A, or a surgical intervention. In some cases, a diagnostic result indicates suitability or non-suitability of a patient for treatment with anti-TNFα. In some cases, a diagnostic result indicates suitability or non-suitability of a patient for treatment with a modulator of miR-155. In some embodiments, the treatment comprises a modulator of a kinase, such as a kinase of Table 20A. In some embodiments, the kinase modulator comprises an agent of Table 20B.

In some embodiments, results are classified using a trained algorithm. Trained algorithms include algorithms that have been developed using a reference set of samples with a known IBD phenotype, such as PBT and CD-PBmu. Algorithms suitable for categorization of samples include but are not limited to k-nearest neighbor algorithms, concept vector algorithms, naive bayesian algorithms, neural network algorithms, hidden markov model algorithms, genetic algorithms, and mutual information feature selection algorithms or any combination thereof. In some cases, trained algorithms may incorporate data other than gene expression such as DNA polymorphism data, sequencing data, scoring or diagnosis by cytologists or pathologists, information provided by the pre-classifier algorithm, or information about the medical history of the subject.

### Compositions and Methods of Treatment

Provided herein are compositions and methods of treating an individual having an inflammatory disease or condition. Non-limiting examples of inflammatory diseases include diseases of the gastrointestinal tract, liver, and/or gallbladder, including Crohn's disease (CD) and ulcerative colitis, systemic lupus erythematosus (SLE), and rheumatoid arthritis. In some embodiments, the subject has a certain phenotype of IBD, such as perianal disease/fistula, stricturing disease, recurrence, or increased immune reactivity to a microbial antigen, or a combination thereof. Compositions include any therapeutic agent that modulates expression and/or activity of a biomolecule in a pathway of one or more markers in Tables 1A-1B, 13, 16, 17A. In some embodiments, the therapeutic agent is a modulator of Adenylate cyclase type 7 (ADCY7), G protein-coupled receptor 65 (GPR65), intercellular adhesion molecule 3 (ICAM3), interferon gamma (IFNGMitogen-activated protein kinase kinase kinase kinase 4 (MAP4K4), E2 receptor EP4 subtype (PTGER4), Receptor-interacting serine/threonine-protein kinase 2 (RIPK2), Ribonuclease T2 (RNASET2), Tumor necrosis factor ligand superfamily member 15 (TNFSF15), or miR-155. As a non-limiting embodiment, the TNFSF15 modulator is an anti-TL1A antibody. In some embodiments, the therapeutic agent is a modulator of a kinase. Non-limiting exemplary kinases include PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, and PKR. Non-limiting examples of kinase targets include those in Table 20A. In some embodiments, a kinase target comprises one or more of the kinases of Table 20A. Non-limiting examples of kinase modulators includes those in Table 20B. In some embodiments, a kinase modulator comprises one or more kinase modulators of Table 20B. In some implementations, the therapeutic agent is administered to a patient determined to have a CD-PBmu subtype as determined by a method provided herein.

In certain embodiments, described herein are methods for evaluating an effect of a treatment described herein. In some instances, the treatment comprises administration with a therapeutic agent provided herein, and optionally one or more additional therapeutic agents. In some instances, the treatment is monitored by evaluating the gene expression profile of a subject for expression of one or more genes in Tables 1A-1B, Table 16, or Table 17A. The gene expression profile may be determined prior to and/or after administration of a therapeutic agent. Gene expression profiling may also be used to ascertain the potential efficacy of a specific therapeutic intervention prior to administering to a subject.

In some embodiments, a therapeutic agent modulates expression and/or activity of ADAMTS1. In some embodiments, a therapeutic agent modulates expression and/or activity of LCN2. In some embodiments, a therapeutic agent modulates expression and/or activity of ADAM28. In some embodiments, a therapeutic agent modulates expression and/or activity of TPSB2. In some embodiments, a therapeutic agent modulates expression and/or activity of PPIAP30. In some embodiments, a therapeutic agent modulates expression and/or activity of GFPT2. In some embodiments, a therapeutic agent modulates expression and/or activity of KIT. In some embodiments, a therapeutic agent modulates expression and/or activity of PLTP. In some embodiments, a therapeutic agent modulates expression and/or activity of MFSD2A. In some embodiments, a therapeutic agent modulates expression and/or activity of IL22. In some embodiments, a therapeutic agent modulates expression and/or activity of LMCD1. In some embodiments, a therapeutic agent modulates expression and/or activity of IL6. In some embodiments, a therapeutic agent modulates expression and/or activity of TBC1D9. In some embodiments, a therapeutic agent modulates expression and/or activity of CHAC1. In some embodiments, a therapeutic agent modulates expression and/or activity of SEPP1. In some embodiments, a therapeutic agent modulates expression and/or activity of SOD3. In some embodiments, a therapeutic agent modulates expression and/or activity of RAB13. In some embodiments, a therapeutic agent modulates expression and/or activity of LYZ. In some embodiments, a therapeutic agent modulates expression and/or activity of CPA3. In some embodiments, a therapeutic agent modulates expression and/or activity of SDS. In some embodiments, a therapeutic agent modulates expression and/or activity of DYRK3. In some embodiments, a therapeutic agent modulates expression and/or activity of DAB2. In some embodiments, a therapeutic agent modulates expression and/or activity of TBC1D8. In some embodiments, a therapeutic agent modulates expression and/or activity of CRYAB. In some embodiments, a therapeutic agent modulates expression and/or activity of TBC1D3. In some embodiments, a therapeutic agent modulates expression and/or activity of LRRC32. In some embodiments, a therapeutic agent modulates expression and/or activity of SERPING1. In some embodiments, a therapeutic agent modulates expression and/or activity of UBD. In some embodiments, a therapeutic agent modulates expression and/or activity of FABP1. In some embodiments, a therapeutic agent modulates expression and/or activity of SYK. In some embodiments, a therapeutic agent modulates expression and/or activity of ALDOB. In some embodiments, a therapeutic agent modulates expression and/or activity of SEMA6B. In some embodiments, a therapeutic agent modulates expression and/or activity of NANOGNB. In some embodiments, a therapeutic agent modulates expression and/or activity of DSE. In some embodiments, a therapeutic agent modulates expression and/or activity of FPR3. In some embodiments, a therapeutic agent modulates expression and/or activity of TNXB. In some embodiments, a therapeutic agent modulates expression and/or activity of OR4A5. In some embodiments, a therapeutic agent modulates expression and/or activity of DCN. In some embodiments, a therapeutic agent modulates expression and/or activity of CHST15. In some embodiments, a therapeutic agent modulates expression and/or activity of ADAMDEC1. In some embodiments, a therapeutic agent modulates expression and/or activity of HDC. In some embodiments, a therapeutic agent modulates expression and/or activity of RRAD. In some embodiments, a therapeutic agent modulates expression and/or activity of C1S. In some embodiments, a therapeutic agent modulates expression and/or activity of PLA2G2A. In some embodiments, a therapeutic agent modulates expression and/or activity of CYCSP52. In some embodiments, a therapeutic agent modulates expression and/or activity of C11orf96. In some embodiments, a therapeutic agent modulates expression and/or activity of SEPSECS-AS1. In some embodiments, a therapeutic agent modulates expression and/or activity of C1QC. In some embodiments, a therapeutic agent modulates expression and/or activity of SLC9B1. In some embodiments, a therapeutic agent modulates expression and/or activity of MLLT10P1. In some embodiments, a therapeutic agent modulates expression and/or activity of LOC102724034. In some embodiments, a therapeutic agent modulates expression and/or activity of SMOX. In some embodiments, a therapeutic agent modulates expression and/or activity of CKB. In some embodiments, a therapeutic agent modulates expression and/or activity of NCOR1P1. In some embodiments, a therapeutic agent modulates expression and/or activity of LOC646736. In some embodiments, a therapeutic agent modulates expression and/or activity of CLEC3B. In some embodiments, a therapeutic agent modulates expression and/or activity of SLCO4A1. In some embodiments, a therapeutic agent modulates expression and/or activity of APOC1P1. In some embodiments, a therapeutic agent modulates expression and/or activity of KGFLP2. In some embodiments, a therapeutic agent modulates expression and/or activity of ABI3BP. In some embodiments, a therapeutic agent modulates expression and/or activity of LINC01189. In some embodiments, a therapeutic agent modulates expression and/or activity of SEPT14. In some embodiments, a therapeutic agent modulates expression and/or activity of FSTL1. In some embodiments, a therapeutic agent modulates expression and/or activity of GEM. In some embodiments, a therapeutic agent modulates expression and/or activity of FAM27A. In some embodiments, a therapeutic agent modulates expression and/or activity of PTENP1-AS. In some embodiments, a therapeutic agent modulates expression and/or activity of LIMS3L. In some embodiments, a therapeutic agent modulates expression and/or activity of ST13P4. In some embodiments, a therapeutic agent modulates expression and/or activity of C1QB. In some embodiments, a therapeutic agent modulates expression and/or activity of HNRNPA1P33. In some embodiments, a therapeutic agent modulates expression and/or activity of MIR663A. In some embodiments, a therapeutic agent modulates expression and/or activity of LOC101927123. In some embodiments, a therapeutic agent modulates expression and/or activity of C2orf27A. In some embodiments, a therapeutic agent modulates expression and/or activity of LOC645166. In some embodiments, a therapeutic agent modulates expression and/or activity of ZNF582-AS1. In some embodiments, a therapeutic agent modulates expression and/or activity of HSPA2. In some embodiments, a therapeutic agent modulates expression and/or activity of COL1A1. In some embodiments, a therapeutic agent modulates expression and/or activity of COL5A1. In some embodiments, a therapeutic agent modulates expression and/or activity of GOLGA6L5P. In some embodiments, a therapeutic agent modulates expression and/or activity of PGM5-AS1. In some embodiments, a therapeutic agent modulates expression and/or activity of CLDN10. In some embodiments, a therapeutic agent modulates expression and/or activity of UBE2Q2L. In some embodiments, a therapeutic agent modulates expression and/or activity of LOC100129138. In some embodiments, a therapeutic agent modulates expression and/or activity of COL1A2. In some embodiments, a therapeutic agent modulates expression and/or activity of SPARCL1. In some embodiments, a therapeutic agent modulates expression and/or activity of FAM222A. In some embodiments, a therapeutic agent modulates expression and/or activity of LINC00857. In some embodiments, a therapeutic agent modulates expression and/or activity of CLIC4. In some embodiments, a therapeutic agent modulates expression and/or activity of FAM182B. In some embodiments, a therapeutic agent modulates expression and/or activity of LOC642426. In some embodiments, a therapeutic agent modulates expression and/or activity of GYPE. In some embodiments, a therapeutic agent modulates expression and/or activity of C8orf4. In some embodiments, a therapeutic agent modulates expression and/or activity of RPSAP9. In some embodiments, a therapeutic agent modulates expression and/or activity of FAM231A. In some embodiments, a therapeutic agent modulates expression and/or activity of LINC00700. In some embodiments, a therapeutic agent modulates expression and/or activity of ANKRD20A3. In some embodiments, a therapeutic agent modulates expression and/or activity of FAM138D. In some embodiments, a therapeutic agent modulates expression and/or activity of KRT20. In some embodiments, a therapeutic agent modulates expression and/or activity of UBTFL1. In some embodiments, a therapeutic agent modulates expression and/or activity of GAS7. In some embodiments, a therapeutic agent modulates expression and/or activity of GPNMB. In some embodiments, a therapeutic agent modulates expression and/or activity of TCF4. In some embodiments, a therapeutic agent modulates expression and/or activity of LINC00348. In some embodiments, a therapeutic agent modulates expression and/or activity of SRC. In some embodiments, a therapeutic agent modulates expression and/or activity of HSPB6. In some embodiments, a therapeutic agent modulates expression and/or activity of LOC100507006. In some embodiments, a therapeutic agent modulates expression and/or activity of TCF21. In some embodiments, a therapeutic agent modulates expression and/or activity of TMEM45B. In some embodiments, a therapeutic agent modulates expression and/or activity of LOC101927905. In some embodiments, a therapeutic agent modulates expression and/or activity of CXCL13. In some embodiments, a therapeutic agent modulates expression and/or activity of AQP7P3. In some embodiments, a therapeutic agent modulates expression and/or activity of PMP22. In some embodiments, a therapeutic agent modulates expression and/or activity of LOC101928163. In some embodiments, a therapeutic agent modulates expression and/or activity of REG3A. In some embodiments, a therapeutic agent modulates expression and/or activity of MMP19. In some embodiments, a therapeutic agent modulates expression and/or activity of PHLDB1. In some embodiments, a therapeutic agent modulates expression and/or activity of LOC100508046. In some embodiments, a therapeutic agent modulates expression and/or activity of SPINK4. In some embodiments, a therapeutic agent modulates expression and/or activity of HES4. In some embodiments, a therapeutic agent modulates expression and/or activity of TREM1. In some embodiments, a therapeutic agent modulates expression and/or activity of TNFRSF12A. In some embodiments, a therapeutic agent modulates expression and/or activity of PRKX-AS1. In some embodiments, a therapeutic agent modulates expression and/or activity of PLGLB1. In some embodiments, a therapeutic agent modulates expression and/or activity of SNAI1. In some embodiments, a therapeutic agent modulates expression and/or activity of NUCB1-AS1. In some embodiments, a therapeutic agent modulates expression and/or activity of BASP1. In some embodiments, a therapeutic agent modulates expression and/or activity of MGP. In some embodiments, a therapeutic agent modulates expression and/or activity of ANPEP. In some embodiments, a therapeutic agent modulates expression and/or activity of PHACTR1. In some embodiments, a therapeutic agent modulates expression and/or activity of ADM. In some embodiments, a therapeutic agent modulates expression and/or activity of DEFA6. In some embodiments, a therapeutic agent modulates expression and/or activity of VEGFA. In some embodiments, a therapeutic agent modulates expression and/or activity of EGR2. In some embodiments, a therapeutic agent modulates expression and/or activity of DEFA5. In some embodiments, a therapeutic agent modulates expression and/or activity of CXCL3. In some embodiments, a therapeutic agent modulates expression and/or activity of SDC4. In some embodiments, a therapeutic agent modulates expression and/or activity of TPSAB1. In some embodiments, a therapeutic agent modulates expression and/or activity of CD68. In some embodiments, a therapeutic agent modulates expression and/or activity of EPAS1. In some embodiments, a therapeutic agent modulates expression and/or activity of MARCKS. In some embodiments, a therapeutic agent modulates expression and/or activity of TNFAIP2. In some embodiments, a therapeutic agent modulates expression and/or activity of MIR663B. In some embodiments, a therapeutic agent modulates expression and/or activity of TMEM114. In some embodiments, a therapeutic agent modulates expression and/or activity of SIRPA. In some embodiments, a therapeutic agent modulates expression and/or activity of GAS6. In some embodiments, a therapeutic agent modulates expression and/or activity of IGFBP7. In some embodiments, a therapeutic agent modulates expression and/or activity of ASB2. In some embodiments, a therapeutic agent modulates expression and/or activity of HES1. In some embodiments, a therapeutic agent modulates expression and/or activity of LOC284801. In some embodiments, a therapeutic agent modulates expression and/or activity of TNFRSF13B. In some embodiments, a therapeutic agent modulates expression and/or activity of MIR548I1. In some embodiments, a therapeutic agent modulates expression and/or activity of DERL3. In some embodiments, a therapeutic agent modulates expression and/or activity of SPARC. In some embodiments, a therapeutic agent modulates expression and/or activity of EMP1. In some embodiments, a therapeutic agent modulates expression and/or activity of LOC100240735. In some embodiments, a therapeutic agent modulates expression and/or activity of LOC101927817. In some embodiments, a therapeutic agent modulates expression and/or activity of STAB 1. In some embodiments, a therapeutic agent modulates expression and/or activity of UPK3B. In some embodiments, a therapeutic agent modulates expression and/or activity of RAB20. In some embodiments, a therapeutic agent modulates expression and/or activity of MMP9. In some embodiments, a therapeutic agent modulates expression and/or activity of MT1G. In some embodiments, a therapeutic agent modulates expression and/or activity of POC1B-GALNT4. In some embodiments, a therapeutic agent modulates expression and/or activity of CSF2RB. In some embodiments, a therapeutic agent modulates expression and/or activity of IL1RN. In some embodiments, a therapeutic agent modulates expression and/or activity of PLEKHA4. In some embodiments, a therapeutic agent modulates expression and/or activity of LOC644172. In some embodiments, a therapeutic agent modulates expression and/or activity of MAFF. In some embodiments, a therapeutic agent modulates expression and/or activity of FDCSP. In some embodiments, a therapeutic agent modulates expression and/or activity of DNASE1L3. In some embodiments, a therapeutic agent modulates expression and/or activity of PTGS2. In some embodiments, a therapeutic agent modulates expression and/or activity of TUBB6. In some embodiments, a therapeutic agent modulates expression and/or activity of LINC01194. In some embodiments, a therapeutic agent modulates expression and/or activity of CTAGE8. In some embodiments, a therapeutic agent modulates expression and/or activity of REG1A. In some embodiments, a therapeutic agent modulates expression and/or activity of ATP5J2-PTCD1. In some embodiments, a therapeutic agent modulates expression and/or activity of DOK3. In some embodiments, a therapeutic agent modulates expression and/or activity of EGR3. In some embodiments, a therapeutic agent modulates expression and/or activity of AOAH-IT1. In some embodiments, a therapeutic agent modulates expression and/or activity of RNASE1. In some embodiments, a therapeutic agent modulates expression and/or activity of CCL11. In some embodiments, a therapeutic agent modulates expression and/or activity of OR4F21. In some embodiments, a therapeutic agent modulates expression and/or activity of FAM157B. In some embodiments, a therapeutic agent modulates expression and/or activity of GATA2. In some embodiments, a therapeutic agent modulates expression and/or activity of CTGF. In some embodiments, a therapeutic agent modulates expression and/or activity of CXCL1. In some embodiments, a therapeutic agent modulates expression and/or activity of GPX3. In some embodiments, a therapeutic agent modulates expression and/or activity of FAM138A. In some embodiments, a therapeutic agent modulates expression and/or activity of FAM138F. In some embodiments, a therapeutic agent modulates expression and/or activity of FOSL1. In some embodiments, a therapeutic agent modulates expression and/or activity of FSCN1. In some embodiments, a therapeutic agent modulates expression and/or activity of FTH1P3. In some embodiments, a therapeutic agent modulates expression and/or activity of SPHK1. In some embodiments, a therapeutic agent modulates expression and/or activity of LOC441242. In some embodiments, a therapeutic agent modulates expression and/or activity of UGT2B10. In some embodiments, a therapeutic agent modulates expression and/or activity of MCTP1. In some embodiments, a therapeutic agent modulates expression and/or activity of IL21R-AS1. In some embodiments, a therapeutic agent modulates expression and/or activity of LOC285740. In some embodiments, a therapeutic agent modulates expression and/or activity of HLA-L. In some embodiments, a therapeutic agent modulates expression and/or activity of NPIPB9. In some embodiments, a therapeutic agent modulates expression and/or activity of SEPT10. In some embodiments, a therapeutic agent modulates expression and/or activity of DNAPK. In some embodiments, a therapeutic agent modulates expression and/or activity of CDK4. In some embodiments, a therapeutic agent modulates expression and/or activity of ERK1. In some embodiments, a therapeutic agent modulates expression and/or activity of HIPK2. In some embodiments, a therapeutic agent modulates expression and/or activity of CDC2. In some embodiments, a therapeutic agent modulates expression and/or activity of MAPK1. In some embodiments, a therapeutic agent modulates expression and/or activity of MAPK3. In some embodiments, a therapeutic agent modulates expression and/or activity of ERK2. In some embodiments, a therapeutic agent modulates expression and/or activity of CSNK2A1. In some embodiments, a therapeutic agent modulates expression and/or activity of CK2ALPHA. In some embodiments, a therapeutic agent modulates expression and/or activity of JNK1. In some embodiments, a therapeutic agent modulates expression and/or activity of CDK1. In some embodiments, a therapeutic agent modulates expression and/or activity of PDK1. In some embodiments, a therapeutic agent modulates expression and/or activity of CDK11B. In some embodiments, a therapeutic agent modulates expression and/or activity of ULK1. In some embodiments, a therapeutic agent modulates expression and/or activity of RIPK1. In some embodiments, a therapeutic agent modulates expression and/or activity of IKBKB. In some embodiments, a therapeutic agent modulates expression and/or activity of CDK9. In some embodiments, a therapeutic agent modulates expression and/or activity of STK11. In some embodiments, a therapeutic agent modulates expression and/or activity of RAF1. In some embodiments, a therapeutic agent modulates expression and/or activity of CSNK1A1. In some embodiments, a therapeutic agent modulates expression and/or activity of AURKB. In some embodiments, a therapeutic agent modulates expression and/or activity of ATR. In some embodiments, a therapeutic agent modulates expression and/or activity of PRKAA2. In some embodiments, a therapeutic agent modulates expression and/or activity of CHEK2. In some embodiments, a therapeutic agent modulates expression and/or activity of PRKDC. In some embodiments, a therapeutic agent modulates expression and/or activity of AURKA. In some embodiments, a therapeutic agent modulates expression and/or activity of RPS6KB1. In some embodiments, a therapeutic agent modulates expression and/or activity of CSNK2A2. In some embodiments, a therapeutic agent modulates expression and/or activity of PLK1. In some embodiments, a therapeutic agent modulates expression and/or activity of PRKAA1. In some embodiments, a therapeutic agent modulates expression and/or activity of MTOR. In some embodiments, a therapeutic agent modulates expression and/or activity of CDK1. In some embodiments, a therapeutic agent modulates expression and/or activity of CDK2. In some embodiments, a therapeutic agent modulates expression and/or activity of MAPK1. In some embodiments, a therapeutic agent modulates expression and/or activity of GSK3B. In some embodiments, a therapeutic agent modulates expression and/or activity of CSNK2A1. In some embodiments, a therapeutic agent modulates expression and/or activity of MAPK14. In some embodiments, a therapeutic agent modulates expression and/or activity of PKR. In some embodiments, a therapeutic agent modulates expression and/or activity of CDK2. In some embodiments, a therapeutic agent modulates expression and/or activity of miR-155. In some embodiments, a therapeutic agent modulates expression and/or activity of ADH4. In some embodiments, a therapeutic agent modulates expression and/or activity of ALG1L. In some embodiments, a therapeutic agent modulates expression and/or activity of BCDIN3D. In some embodiments, a therapeutic agent modulates expression and/or activity of C1orf106. In some embodiments, a therapeutic agent modulates expression and/or activity of C2. In some embodiments, a therapeutic agent modulates expression and/or activity of CCDC144NL. In some embodiments, a therapeutic agent modulates expression and/or activity of CEACAM5. In some embodiments, a therapeutic agent modulates expression and/or activity of CTAGE8. In some embodiments, a therapeutic agent modulates expression and/or activity of DDX11L2. In some embodiments, a therapeutic agent modulates expression and/or activity of DPPA4. In some embodiments, a therapeutic agent modulates expression and/or activity of DUSP19. In some embodiments, a therapeutic agent modulates expression and/or activity of FGB. In some embodiments, a therapeutic agent modulates expression and/or activity of GP2. In some embodiments, a therapeutic agent modulates expression and/or activity of GYPE. In some embodiments, a therapeutic agent modulates expression and/or activity of HSD3B7. In some embodiments, a therapeutic agent modulates expression and/or activity of HUNK. In some embodiments, a therapeutic agent modulates expression and/or activity of JAM2. In some embodiments, a therapeutic agent modulates expression and/or activity of KCNE3. In some embodiments, a therapeutic agent modulates expression and/or activity of KRT42P. In some embodiments, a therapeutic agent modulates expression and/or activity of LYZ. In some embodiments, a therapeutic agent modulates expression and/or activity of MLLT10P1. In some embodiments, a therapeutic agent modulates expression and/or activity of NAP1L6. In some embodiments, a therapeutic agent modulates expression and/or activity of NEURL3. In some embodiments, a therapeutic agent modulates expression and/or activity of NPIPB9. In some embodiments, a therapeutic agent modulates expression and/or activity of PANK1. In some embodiments, a therapeutic agent modulates expression and/or activity of PKIB. In some embodiments, a therapeutic agent modulates expression and/or activity of RHOU. In some embodiments, a therapeutic agent modulates expression and/or activity of RPSAP9. In some embodiments, a therapeutic agent modulates expression and/or activity of SHCBP1. In some embodiments, a therapeutic agent modulates expression and/or activity of SIGLEC8. In some embodiments, a therapeutic agent modulates expression and/or activity of SLC15A2. In some embodiments, a therapeutic agent modulates expression and/or activity of SLC25A34. In some embodiments, a therapeutic agent modulates expression and/or activity of SLC6A20. In some embodiments, a therapeutic agent modulates expression and/or activity of SLC9B1. In some embodiments, a therapeutic agent modulates expression and/or activity of SYNPO2L. In some embodiments, a therapeutic agent modulates expression and/or activity of TDGF1. In some embodiments, a therapeutic agent modulates expression and/or activity of ZNF491. In some embodiments, a therapeutic agent modulates expression and/or activity of ZNF620. In some embodiments, a therapeutic agent modulates expression and/or activity of ZNF69. In some embodiments, a therapeutic agent modulates expression and/or activity of CXCL16. In some embodiments, a therapeutic agent modulates expression and/or activity of CD68. In some embodiments, a therapeutic agent modulates expression and/or activity of CD300E.

In some embodiments, a therapeutic agent modulates expression and/or activity of a biomolecule in a pathway comprising ADAMTS1. In some embodiments, a therapeutic agent modulates expression and/or activity of a biomolecule in a pathway comprising LCN2. In some embodiments, a therapeutic agent modulates expression and/or activity of a biomolecule in a pathway comprising ADAM28. In some embodiments, a therapeutic agent modulates expression and/or activity of a biomolecule in a pathway comprising TPSB2. In some embodiments, a therapeutic agent modulates expression and/or activity of a biomolecule in a pathway comprising PPIAP30. In some embodiments, a therapeutic agent modulates expression and/or activity of a biomolecule in a pathway comprising GFPT2. In some embodiments, a therapeutic agent modulates expression and/or activity of a biomolecule in a pathway comprising KIT. In some embodiments, a therapeutic agent modulates expression and/or activity of a biomolecule in a pathway comprising PLTP. In some embodiments, a therapeutic agent modulates expression and/or activity of a biomolecule in a pathway comprising MFSD2A. In some embodiments, a therapeutic agent modulates expression and/or activity of a biomolecule in a pathway comprising IL22. In some embodiments, a therapeutic agent modulates expression and/or activity of a biomolecule in a pathway comprising LMCD1. In some embodiments, a therapeutic agent modulates expression and/or activity of a biomolecule in a pathway comprising IL6. In some embodiments, a therapeutic agent modulates expression and/or activity of a biomolecule in a pathway comprising TBC1D9. In some embodiments, a therapeutic agent modulates expression and/or activity of a biomolecule in a pathway comprising CHAC1. In some embodiments, a therapeutic agent modulates expression and/or activity of a biomolecule in a pathway comprising SEPP1. In some embodiments, a therapeutic agent modulates expression and/or activity of a biomolecule in a pathway comprising SOD3. In some embodiments, a therapeutic agent modulates expression and/or activity of a biomolecule in a pathway comprising RAB13. In some embodiments, a therapeutic agent modulates expression and/or activity of a biomolecule in a pathway comprising LYZ. In some embodiments, a therapeutic agent modulates expression and/or activity of a biomolecule in a pathway comprising CPA3. In some embodiments, a therapeutic agent modulates expression and/or activity of a biomolecule in a pathway comprising SDS. In some embodiments, a therapeutic agent modulates expression and/or activity of a biomolecule in a pathway comprising DYRK3. In some embodiments, a therapeutic agent modulates expression and/or activity of a biomolecule in a pathway comprising DAB2. In some embodiments, a therapeutic agent modulates expression and/or activity of a biomolecule in a pathway comprising TBC1D8. In some embodiments, a therapeutic agent modulates expression and/or activity of a biomolecule in a pathway comprising CRYAB. In some embodiments, a therapeutic agent modulates expression and/or activity of a biomolecule in a pathway comprising TBC1D3. In some embodiments, a therapeutic agent modulates expression and/or activity of a biomolecule in a pathway comprising LRRC32. In some embodiments, a therapeutic agent modulates expression and/or activity of a biomolecule in a pathway comprising SERPING1. In some embodiments, a therapeutic agent modulates expression and/or activity of a biomolecule in a pathway comprising UBD. In some embodiments, a therapeutic agent modulates expression and/or activity of a biomolecule in a pathway comprising FABP1. In some embodiments, a therapeutic agent modulates expression and/or activity of a biomolecule in a pathway comprising SYK. In some embodiments, a therapeutic agent modulates expression and/or activity of a biomolecule in a pathway comprising ALDOB. In some embodiments, a therapeutic agent modulates expression and/or activity of a biomolecule in a pathway comprising SEMA6B. In some embodiments, a therapeutic agent modulates expression and/or activity of a biomolecule in a pathway comprising NANOGNB. In some embodiments, a therapeutic agent modulates expression and/or activity of a biomolecule in a pathway comprising DSE. In some embodiments, a therapeutic agent modulates expression and/or activity of a biomolecule in a pathway comprising FPR3. In some embodiments, a therapeutic agent modulates expression and/or activity of a biomolecule in a pathway comprising TNXB. In some embodiments, a therapeutic agent modulates expression and/or activity of a biomolecule in a pathway comprising OR4A5. In some embodiments, a therapeutic agent modulates expression and/or activity of a biomolecule in a pathway comprising DCN. In some embodiments, a therapeutic agent modulates expression and/or activity of a biomolecule in a pathway comprising CHST15. In some embodiments, a therapeutic agent modulates expression and/or activity of a biomolecule in a pathway comprising ADAMDEC1. In some embodiments, a therapeutic agent modulates expression and/or activity of a biomolecule in a pathway comprising HDC. In some embodiments, a therapeutic agent modulates expression and/or activity of a biomolecule in a pathway comprising RRAD. In some embodiments, a therapeutic agent modulates expression and/or activity of a biomolecule in a pathway comprising C1S. In some embodiments, a therapeutic agent modulates expression and/or activity of a biomolecule in a pathway comprising PLA2G2A. In some embodiments, a therapeutic agent modulates expression and/or activity of a biomolecule in a pathway comprising CYCSP52. In some embodiments, a therapeutic agent modulates expression and/or activity of a biomolecule in a pathway comprising C11orf96. In some embodiments, a therapeutic agent modulates expression and/or activity of a biomolecule in a pathway comprising SEPSECS-AS1. In some embodiments, a therapeutic agent modulates expression and/or activity of a biomolecule in a pathway comprising C1QC. In some embodiments, a therapeutic agent modulates expression and/or activity of a biomolecule in a pathway comprising SLC9B1. In some embodiments, a therapeutic agent modulates expression and/or activity of a biomolecule in a pathway comprising MLLT10P1. In some embodiments, a therapeutic agent modulates expression and/or activity of a biomolecule in a pathway comprising LOC102724034. In some embodiments, a therapeutic agent modulates expression and/or activity of a biomolecule in a pathway comprising SMOX. In some embodiments, a therapeutic agent modulates expression and/or activity of a biomolecule in a pathway comprising CKB. In some embodiments, a therapeutic agent modulates expression and/or activity of a biomolecule in a pathway comprising NCOR1P1. In some embodiments, a therapeutic agent modulates expression and/or activity of a biomolecule in a pathway comprising LOC646736. In some embodiments, a therapeutic agent modulates expression and/or activity of a biomolecule in a pathway comprising CLEC3B. In some embodiments, a therapeutic agent modulates expression and/or activity of a biomolecule in a pathway comprising SLCO4A1. In some embodiments, a therapeutic agent modulates expression and/or activity of a biomolecule in a pathway comprising APOC1P1. In some embodiments, a therapeutic agent modulates expression and/or activity of a biomolecule in a pathway comprising KGFLP2. In some embodiments, a therapeutic agent modulates expression and/or activity of a biomolecule in a pathway comprising ABI3BP. In some embodiments, a therapeutic agent modulates expression and/or activity of a biomolecule in a pathway comprising LINC01189. In some embodiments, a therapeutic agent modulates expression and/or activity of a biomolecule in a pathway comprising SEPT14. In some embodiments, a therapeutic agent modulates expression and/or activity of a biomolecule in a pathway comprising FSTL1. In some embodiments, a therapeutic agent modulates expression and/or activity of a biomolecule in a pathway comprising GEM. In some embodiments, a therapeutic agent modulates expression and/or activity of a biomolecule in a pathway comprising FAM27A. In some embodiments, a therapeutic agent modulates expression and/or activity of a biomolecule in a pathway comprising PTENP1-AS. In some embodiments, a therapeutic agent modulates expression and/or activity of a biomolecule in a pathway comprising LIMS3L. In some embodiments, a therapeutic agent modulates expression and/or activity of a biomolecule in a pathway comprising ST13P4. In some embodiments, a therapeutic agent modulates expression and/or activity of a biomolecule in a pathway comprising C1QB. In some embodiments, a therapeutic agent modulates expression and/or activity of a biomolecule in a pathway comprising HNRNPA1P33. In some embodiments, a therapeutic agent modulates expression and/or activity of a biomolecule in a pathway comprising MIR663A. In some embodiments, a therapeutic agent modulates expression and/or activity of a biomolecule in a pathway comprising LOC101927123. In some embodiments, a therapeutic agent modulates expression and/or activity of a biomolecule in a pathway comprising C2orf27A. In some embodiments, a therapeutic agent modulates expression and/or activity of a biomolecule in a pathway comprising LOC645166. In some embodiments, a therapeutic agent modulates expression and/or activity of a biomolecule in a pathway comprising ZNF582-AS1. In some embodiments, a therapeutic agent modulates expression and/or activity of a biomolecule in a pathway comprising HSPA2. In some embodiments, a therapeutic agent modulates expression and/or activity of a biomolecule in a pathway comprising COL1A1. In some embodiments, a therapeutic agent modulates expression and/or activity of a biomolecule in a pathway comprising COL5A1. In some embodiments, a therapeutic agent modulates expression and/or activity of a biomolecule in a pathway comprising GOLGA6L5P. In some embodiments, a therapeutic agent modulates expression and/or activity of a biomolecule in a pathway comprising PGM5-AS1. In some embodiments, a therapeutic agent modulates expression and/or activity of a biomolecule in a pathway comprising CLDN10. In some embodiments, a therapeutic agent modulates expression and/or activity of a biomolecule in a pathway comprising UBE2Q2L. In some embodiments, a therapeutic agent modulates expression and/or activity of a biomolecule in a pathway comprising LOC100129138. In some embodiments, a therapeutic agent modulates expression and/or activity of a biomolecule in a pathway comprising COL1A2. In some embodiments, a therapeutic agent modulates expression and/or activity of a biomolecule in a pathway comprising SPARCL1. In some embodiments, a therapeutic agent modulates expression and/or activity of a biomolecule in a pathway comprising FAM222A. In some embodiments, a therapeutic agent modulates expression and/or activity of a biomolecule in a pathway comprising LINC00857. In some embodiments, a therapeutic agent modulates expression and/or activity of a biomolecule in a pathway comprising CLIC4. In some embodiments, a therapeutic agent modulates expression and/or activity of a biomolecule in a pathway comprising FAM182B. In some embodiments, a therapeutic agent modulates expression and/or activity of a biomolecule in a pathway comprising LOC642426. In some embodiments, a therapeutic agent modulates expression and/or activity of a biomolecule in a pathway comprising GYPE. In some embodiments, a therapeutic agent modulates expression and/or activity of a biomolecule in a pathway comprising C8orf4. In some embodiments, a therapeutic agent modulates expression and/or activity of a biomolecule in a pathway comprising RPSAP9. In some embodiments, a therapeutic agent modulates expression and/or activity of a biomolecule in a pathway comprising FAM231A. In some embodiments, a therapeutic agent modulates expression and/or activity of a biomolecule in a pathway comprising LINC00700. In some embodiments, a therapeutic agent modulates expression and/or activity of a biomolecule in a pathway comprising ANKRD20A3. In some embodiments, a therapeutic agent modulates expression and/or activity of a biomolecule in a pathway comprising FAM138D. In some embodiments, a therapeutic agent modulates expression and/or activity of a biomolecule in a pathway comprising KRT20. In some embodiments, a therapeutic agent modulates expression and/or activity of a biomolecule in a pathway comprising UBTFL1. In some embodiments, a therapeutic agent modulates expression and/or activity of a biomolecule in a pathway comprising GAS7. In some embodiments, a therapeutic agent modulates expression and/or activity of a biomolecule in a pathway comprising GPNMB. In some embodiments, a therapeutic agent modulates expression and/or activity of a biomolecule in a pathway comprising TCF4. In some embodiments, a therapeutic agent modulates expression and/or activity of a biomolecule in a pathway comprising LINC00348. In some embodiments, a therapeutic agent modulates expression and/or activity of a biomolecule in a pathway comprising SRC. In some embodiments, a therapeutic agent modulates expression and/or activity of a biomolecule in a pathway comprising HSPB6. In some embodiments, a therapeutic agent modulates expression and/or activity of a biomolecule in a pathway comprising LOC100507006. In some embodiments, a therapeutic agent modulates expression and/or activity of a biomolecule in a pathway comprising TCF21. In some embodiments, a therapeutic agent modulates expression and/or activity of a biomolecule in a pathway comprising TMEM45B. In some embodiments, a therapeutic agent modulates expression and/or activity of a biomolecule in a pathway comprising LOC101927905. In some embodiments, a therapeutic agent modulates expression and/or activity of a biomolecule in a pathway comprising CXCL13. In some embodiments, a therapeutic agent modulates expression and/or activity of a biomolecule in a pathway comprising AQP7P3. In some embodiments, a therapeutic agent modulates expression and/or activity of a biomolecule in a pathway comprising PMP22. In some embodiments, a therapeutic agent modulates expression and/or activity of a biomolecule in a pathway comprising LOC101928163. In some embodiments, a therapeutic agent modulates expression and/or activity of a biomolecule in a pathway comprising REG3A. In some embodiments, a therapeutic agent modulates expression and/or activity of a biomolecule in a pathway comprising MMP19. In some embodiments, a therapeutic agent modulates expression and/or activity of a biomolecule in a pathway comprising PHLDB1. In some embodiments, a therapeutic agent modulates expression and/or activity of a biomolecule in a pathway comprising LOC100508046. In some embodiments, a therapeutic agent modulates expression and/or activity of a biomolecule in a pathway comprising SPINK4. In some embodiments, a therapeutic agent modulates expression and/or activity of a biomolecule in a pathway comprising HES4. In some embodiments, a therapeutic agent modulates expression and/or activity of a biomolecule in a pathway comprising TREM1. In some embodiments, a therapeutic agent modulates expression and/or activity of a biomolecule in a pathway comprising TNFRSF12A. In some embodiments, a therapeutic agent modulates expression and/or activity of a biomolecule in a pathway comprising PRKX-AS1. In some embodiments, a therapeutic agent modulates expression and/or activity of a biomolecule in a pathway comprising PLGLB1. In some embodiments, a therapeutic agent modulates expression and/or activity of a biomolecule in a pathway comprising SNAI1. In some embodiments, a therapeutic agent modulates expression and/or activity of a biomolecule in a pathway comprising NUCB1-AS1. In some embodiments, a therapeutic agent modulates expression and/or activity of a biomolecule in a pathway comprising BASP1. In some embodiments, a therapeutic agent modulates expression and/or activity of a biomolecule in a pathway comprising MGP. In some embodiments, a therapeutic agent modulates expression and/or activity of a biomolecule in a pathway comprising ANPEP. In some embodiments, a therapeutic agent modulates expression and/or activity of a biomolecule in a pathway comprising PHACTR1. In some embodiments, a therapeutic agent modulates expression and/or activity of a biomolecule in a pathway comprising ADM. In some embodiments, a therapeutic agent modulates expression and/or activity of a biomolecule in a pathway comprising DEFA6. In some embodiments, a therapeutic agent modulates expression and/or activity of a biomolecule in a pathway comprising VEGFA. In some embodiments, a therapeutic agent modulates expression and/or activity of a biomolecule in a pathway comprising EGR2. In some embodiments, a therapeutic agent modulates expression and/or activity of a biomolecule in a pathway comprising DEFA5. In some embodiments, a therapeutic agent modulates expression and/or activity of a biomolecule in a pathway comprising CXCL3. In some embodiments, a therapeutic agent modulates expression and/or activity of a biomolecule in a pathway comprising SDC4. In some embodiments, a therapeutic agent modulates expression and/or activity of a biomolecule in a pathway comprising TPSAB1. In some embodiments, a therapeutic agent modulates expression and/or activity of a biomolecule in a pathway comprising CD68. In some embodiments, a therapeutic agent modulates expression and/or activity of a biomolecule in a pathway comprising EPAS1. In some embodiments, a therapeutic agent modulates expression and/or activity of a biomolecule in a pathway comprising MARCKS. In some embodiments, a therapeutic agent modulates expression and/or activity of a biomolecule in a pathway comprising TNFAIP2. In some embodiments, a therapeutic agent modulates expression and/or activity of a biomolecule in a pathway comprising MIR663B. In some embodiments, a therapeutic agent modulates expression and/or activity of a biomolecule in a pathway comprising TMEM114. In some embodiments, a therapeutic agent modulates expression and/or activity of a biomolecule in a pathway comprising SIRPA. In some embodiments, a therapeutic agent modulates expression and/or activity of a biomolecule in a pathway comprising GAS6. In some embodiments, a therapeutic agent modulates expression and/or activity of a biomolecule in a pathway comprising IGFBP7. In some embodiments, a therapeutic agent modulates expression and/or activity of a biomolecule in a pathway comprising ASB2. In some embodiments, a therapeutic agent modulates expression and/or activity of a biomolecule in a pathway comprising HES1. In some embodiments, a therapeutic agent modulates expression and/or activity of a biomolecule in a pathway comprising LOC284801. In some embodiments, a therapeutic agent modulates expression and/or activity of a biomolecule in a pathway comprising TNFRSF13B. In some embodiments, a therapeutic agent modulates expression and/or activity of a biomolecule in a pathway comprising MIR548I1. In some embodiments, a therapeutic agent modulates expression and/or activity of a biomolecule in a pathway comprising DERL3. In some embodiments, a therapeutic agent modulates expression and/or activity of a biomolecule in a pathway comprising SPARC. In some embodiments, a therapeutic agent modulates expression and/or activity of a biomolecule in a pathway comprising EMP1. In some embodiments, a therapeutic agent modulates expression and/or activity of a biomolecule in a pathway comprising LOC100240735. In some embodiments, a therapeutic agent modulates expression and/or activity of a biomolecule in a pathway comprising LOC101927817. In some embodiments, a therapeutic agent modulates expression and/or activity of a biomolecule in a pathway comprising STAB 1. In some embodiments, a therapeutic agent modulates expression and/or activity of a biomolecule in a pathway comprising UPK3B. In some embodiments, a therapeutic agent modulates expression and/or activity of a biomolecule in a pathway comprising RAB20. In some embodiments, a therapeutic agent modulates expression and/or activity of a biomolecule in a pathway comprising MMP9. In some embodiments, a therapeutic agent modulates expression and/or activity of a biomolecule in a pathway comprising MT1G. In some embodiments, a therapeutic agent modulates expression and/or activity of a biomolecule in a pathway comprising POC1B-GALNT4. In some embodiments, a therapeutic agent modulates expression and/or activity of a biomolecule in a pathway comprising CSF2RB. In some embodiments, a therapeutic agent modulates expression and/or activity of a biomolecule in a pathway comprising IL1RN. In some embodiments, a therapeutic agent modulates expression and/or activity of a biomolecule in a pathway comprising PLEKHA4. In some embodiments, a therapeutic agent modulates expression and/or activity of a biomolecule in a pathway comprising LOC644172. In some embodiments, a therapeutic agent modulates expression and/or activity of a biomolecule in a pathway comprising MAFF. In some embodiments, a therapeutic agent modulates expression and/or activity of a biomolecule in a pathway comprising FDCSP. In some embodiments, a therapeutic agent modulates expression and/or activity of a biomolecule in a pathway comprising DNASE1L3. In some embodiments, a therapeutic agent modulates expression and/or activity of a biomolecule in a pathway comprising PTGS2. In some embodiments, a therapeutic agent modulates expression and/or activity of a biomolecule in a pathway comprising TUBB6. In some embodiments, a therapeutic agent modulates expression and/or activity of a biomolecule in a pathway comprising LINC01194. In some embodiments, a therapeutic agent modulates expression and/or activity of a biomolecule in a pathway comprising CTAGE8. In some embodiments, a therapeutic agent modulates expression and/or activity of a biomolecule in a pathway comprising REG1A. In some embodiments, a therapeutic agent modulates expression and/or activity of a biomolecule in a pathway comprising ATP5J2-PTCD1. In some embodiments, a therapeutic agent modulates expression and/or activity of a biomolecule in a pathway comprising DOK3. In some embodiments, a therapeutic agent modulates expression and/or activity of a biomolecule in a pathway comprising EGR3. In some embodiments, a therapeutic agent modulates expression and/or activity of a biomolecule in a pathway comprising AOAH-IT1. In some embodiments, a therapeutic agent modulates expression and/or activity of a biomolecule in a pathway comprising RNASE1. In some embodiments, a therapeutic agent modulates expression and/or activity of a biomolecule in a pathway comprising CCL11. In some embodiments, a therapeutic agent modulates expression and/or activity of a biomolecule in a pathway comprising OR4F21. In some embodiments, a therapeutic agent modulates expression and/or activity of a biomolecule in a pathway comprising FAM157B. In some embodiments, a therapeutic agent modulates expression and/or activity of a biomolecule in a pathway comprising GATA2. In some embodiments, a therapeutic agent modulates expression and/or activity of a biomolecule in a pathway comprising CTGF. In some embodiments, a therapeutic agent modulates expression and/or activity of a biomolecule in a pathway comprising CXCL1. In some embodiments, a therapeutic agent modulates expression and/or activity of a biomolecule in a pathway comprising GPX3. In some embodiments, a therapeutic agent modulates expression and/or activity of a biomolecule in a pathway comprising FAM138A. In some embodiments, a therapeutic agent modulates expression and/or activity of a biomolecule in a pathway comprising FAM138F. In some embodiments, a therapeutic agent modulates expression and/or activity of a biomolecule in a pathway comprising FOSL1. In some embodiments, a therapeutic agent modulates expression and/or activity of a biomolecule in a pathway comprising FSCN1. In some embodiments, a therapeutic agent modulates expression and/or activity of a biomolecule in a pathway comprising FTH1P3. In some embodiments, a therapeutic agent modulates expression and/or activity of a biomolecule in a pathway comprising SPHK1. In some embodiments, a therapeutic agent modulates expression and/or activity of a biomolecule in a pathway comprising LOC441242. In some embodiments, a therapeutic agent modulates expression and/or activity of a biomolecule in a pathway comprising UGT2B10. In some embodiments, a therapeutic agent modulates expression and/or activity of a biomolecule in a pathway comprising MCTP1. In some embodiments, a therapeutic agent modulates expression and/or activity of a biomolecule in a pathway comprising IL21R-AS1. In some embodiments, a therapeutic agent modulates expression and/or activity of a biomolecule in a pathway comprising LOC285740. In some embodiments, a therapeutic agent modulates expression and/or activity of a biomolecule in a pathway comprising HLA-L. In some embodiments, a therapeutic agent modulates expression and/or activity of a biomolecule in a pathway comprising NPIPB9. In some embodiments, a therapeutic agent modulates expression and/or activity of a biomolecule in a pathway comprising SEPT10. In some embodiments, a therapeutic agent modulates expression and/or activity of a biomolecule in a pathway comprising miR-155. In some embodiments, a therapeutic agent modulates expression and/or activity of a biomolecule in a pathway comprising ADH4. In some embodiments, a therapeutic agent modulates expression and/or activity of a biomolecule in a pathway comprising ALG1L. In some embodiments, a therapeutic agent modulates expression and/or activity of a biomolecule in a pathway comprising BCDIN3D. In some embodiments, a therapeutic agent modulates expression and/or activity of a biomolecule in a pathway comprising C1orf106. In some embodiments, a therapeutic agent modulates expression and/or activity of a biomolecule in a pathway comprising C2. In some embodiments, a therapeutic agent modulates expression and/or activity of a biomolecule in a pathway comprising CCDC144NL. In some embodiments, a therapeutic agent modulates expression and/or activity of a biomolecule in a pathway comprising CEACAM5. In some embodiments, a therapeutic agent modulates expression and/or activity of a biomolecule in a pathway comprising CTAGE8. In some embodiments, a therapeutic agent modulates expression and/or activity of a biomolecule in a pathway comprising DDX11L2. In some embodiments, a therapeutic agent modulates expression and/or activity of a biomolecule in a pathway comprising DPPA4. In some embodiments, a therapeutic agent modulates expression and/or activity of a biomolecule in a pathway comprising DUSP19. In some embodiments, a therapeutic agent modulates expression and/or activity of a biomolecule in a pathway comprising FGB. In some embodiments, a therapeutic agent modulates expression and/or activity of a biomolecule in a pathway comprising GP2. In some embodiments, a therapeutic agent modulates expression and/or activity of a biomolecule in a pathway comprising GYPE. In some embodiments, a therapeutic agent modulates expression and/or activity of a biomolecule in a pathway comprising HSD3B7. In some embodiments, a therapeutic agent modulates expression and/or activity of a biomolecule in a pathway comprising HUNK. In some embodiments, a therapeutic agent modulates expression and/or activity of a biomolecule in a pathway comprising JAM2. In some embodiments, a therapeutic agent modulates expression and/or activity of a biomolecule in a pathway comprising KCNE3. In some embodiments, a therapeutic agent modulates expression and/or activity of a biomolecule in a pathway comprising KRT42P. In some embodiments, a therapeutic agent modulates expression and/or activity of a biomolecule in a pathway comprising LYZ. In some embodiments, a therapeutic agent modulates expression and/or activity of a biomolecule in a pathway comprising MLLT10P1. In some embodiments, a therapeutic agent modulates expression and/or activity of a biomolecule in a pathway comprising NAP1L6. In some embodiments, a therapeutic agent modulates expression and/or activity of a biomolecule in a pathway comprising NEURL3. In some embodiments, a therapeutic agent modulates expression and/or activity of a biomolecule in a pathway comprising NPIPB9. In some embodiments, a therapeutic agent modulates expression and/or activity of a biomolecule in a pathway comprising PANK1. In some embodiments, a therapeutic agent modulates expression and/or activity of a biomolecule in a pathway comprising PKIB. In some embodiments, a therapeutic agent modulates expression and/or activity of a biomolecule in a pathway comprising RHOU. In some embodiments, a therapeutic agent modulates expression and/or activity of a biomolecule in a pathway comprising RPSAP9. In some embodiments, a therapeutic agent modulates expression and/or activity of a biomolecule in a pathway comprising SHCBP1. In some embodiments, a therapeutic agent modulates expression and/or activity of a biomolecule in a pathway comprising SIGLEC8. In some embodiments, a therapeutic agent modulates expression and/or activity of a biomolecule in a pathway comprising SLC15A2. In some embodiments, a therapeutic agent modulates expression and/or activity of a biomolecule in a pathway comprising SLC25A34. In some embodiments, a therapeutic agent modulates expression and/or activity of a biomolecule in a pathway comprising SLC6A20. In some embodiments, a therapeutic agent modulates expression and/or activity of a biomolecule in a pathway comprising SLC9B1. In some embodiments, a therapeutic agent modulates expression and/or activity of a biomolecule in a pathway comprising SYNPO2L. In some embodiments, a therapeutic agent modulates expression and/or activity of a biomolecule in a pathway comprising TDGF1. In some embodiments, a therapeutic agent modulates expression and/or activity of a biomolecule in a pathway comprising ZNF491. In some embodiments, a therapeutic agent modulates expression and/or activity of a biomolecule in a pathway comprising ZNF620. In some embodiments, a therapeutic agent modulates expression and/or activity of a biomolecule in a pathway comprising ZNF69. In some embodiments, a therapeutic agent modulates expression and/or activity of a biomolecule in a pathway comprising CXCL16. In some embodiments, a therapeutic agent modulates expression and/or activity of a biomolecule in a pathway comprising CD68. In some embodiments, a therapeutic agent modulates expression and/or activity of a biomolecule in a pathway comprising CD300E.

### TNF Superfamily Member 15 (TL1A) TL1A Modulators

In some embodiments, the therapeutic agent comprises a modulator and/or antagonist of TNF Superfamily Member 15 (TL1A), or the gene encoding TL1A (TNFSF15). In some embodiments, the modulator of TL1A is an antagonist of TL1A. In some embodiments the therapeutic agent or the additional therapeutic agent comprises an inhibitor of TL1A expression or activity. In some embodiments the therapeutic agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the anti-TL1A antibody is an anti-TL1A antibody that specifically binds to TL1A.

The anti-TL1A antibody may comprise one or more of the antibody sequences of Table 18. The anti-DR3 antibody may comprise an amino acid sequence that is at least 85% identical to any one of SEQ ID NOS: 358-370 and an amino acid sequence that is at least 85% identical to any one of SEQ ID NOS: 371-375. The anti-DR3 antibody may comprise an amino acid sequence comprising the HCDR1, HCDR2, HCDR3 domains of any one of SEQ ID NOS: 358-370 and the LCDR1, LCDR2, and LCDR3 domains of any one of SEQ ID NOS: 371-375.

In some embodiments, an anti-TL1A antibody comprises a heavy chain comprising three complementarity-determining regions: HCDR1, HCDR2, and HCDR3; and a light chain comprising three complementarity-determining regions: LCDR1, LCDR2, and LCDR3. In some embodiments, the anti-TL1A antibody comprises a HCDR1 comprising SEQ ID NO: 209, a HCDR2 comprising SEQ ID NO: 210, a HCDR3 comprising SEQ ID NO: 211, a LCDR1 comprising SEQ ID NO: 212, a LCDR2 comprising SEQ ID NO: 213, and a LCDR3 comprising SEQ ID NO: 214. In some cases, the anti-TL1A antibody comprises a heavy chain (HC) variable domain comprising SEQ ID NO: 215 and a light chain (LC) variable domain comprising SEQ ID NO: 216.

In some embodiments, the anti-TL1A antibody comprises a HCDR1 comprising SEQ ID NO: 217, a HCDR2 comprising SEQ ID NO: 218, a HCDR3 comprising SEQ ID NO: 219, a LCDR1 comprising SEQ ID NO: 220, a LCDR2 comprising SEQ ID NO: 221, and a LCDR3 comprising SEQ ID NO: 222. In some cases, the anti-TL1A antibody comprises a heavy chain (HC) variable domain comprising SEQ ID NO: 223 and a light chain (LC) variable domain comprising SEQ ID NO: 224.

In some embodiments, the anti-TL1A antibody comprises a HCDR1 comprising SEQ ID NO: 225, a HCDR2 comprising SEQ ID NO: 226, a HCDR3 comprising SEQ ID NO: 227, a LCDR1 comprising SEQ ID NO: 228, a LCDR2 comprising SEQ ID NO: 229, and a LCDR3 comprising SEQ ID NO: 230. In some cases, the anti-TL1A antibody comprises a heavy chain (HC) variable domain comprising SEQ ID NO: 231 and a light chain (LC) variable domain comprising SEQ ID NO: 232.

In some embodiments, the anti-TL1A antibody comprises a HCDR1 comprising SEQ ID NO: 233, a HCDR2 comprising SEQ ID NO: 234, a HCDR3 comprising SEQ ID NO: 235, a LCDR1 comprising SEQ ID NO: 239, a LCDR2 comprising SEQ ID NO: 240, and a LCDR3 comprising SEQ ID NO: 241. In some cases, the anti-TL1A antibody comprises a HCDR1 comprising SEQ ID NO: 236, a HCDR2 comprising SEQ ID NO: 237, a HCDR3 comprising SEQ ID NO: 238, a LCDR1 comprising SEQ ID NO: 239, a LCDR2 comprising SEQ ID NO: 240, and a LCDR3 comprising SEQ ID NO: 241. In some cases, the anti-TL1A antibody comprises a heavy chain (HC) variable domain comprising SEQ ID NO: 242 and a light chain (LC) variable domain comprising SEQ ID NO: 243. In some cases, the anti-TL1A antibody comprises a heavy chain comprising SEQ ID NO: 244. In some cases, the anti-TL1A antibody comprises a light chain comprising SEQ ID NO: 245.

In some embodiments, the anti-TL1A antibody comprises a HCDR1 comprising SEQ ID NO: 246, a HCDR2 comprising SEQ ID NO: 247, a HCDR3 comprising SEQ ID NO: 248, a LCDR1 comprising SEQ ID NO: 249, a LCDR2 comprising SEQ ID NO: 250, and a LCDR3 comprising SEQ ID NO: 251. In some cases, the anti-TL1A antibody comprises a heavy chain (HC) variable domain comprising SEQ ID NO: 252 and a light chain (LC) variable domain comprising SEQ ID NO: 253.

In some embodiments, the anti-TL1A antibody comprises a HCDR1 comprising SEQ ID NO: 254, a HCDR2 comprising SEQ ID NO: 255, a HCDR3 comprising SEQ ID NO: 256, a LCDR1 comprising SEQ ID NO: 257, a LCDR2 comprising SEQ ID NO: 258, and a LCDR3 comprising SEQ ID NO: 259. In some cases, the anti-TL1A antibody comprises a heavy chain (HC) variable domain comprising SEQ ID NO: 260 and a light chain (LC) variable domain comprising SEQ ID NO: 261.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 275. In some cases, the anti-TL1A antibody comprises a heavy chain (HC) variable domain comprising SEQ ID NO: 271 and a light chain (LC) variable domain comprising SEQ ID NO: 276. In some cases, the anti-TL1A antibody comprises a heavy chain (HC) variable domain comprising SEQ ID NO: 271 and a light chain (LC) variable domain comprising SEQ ID NO: 277. In some cases, the anti-TL1A antibody comprises a heavy chain (HC) variable domain comprising SEQ ID NO: 271 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 279. In some cases, the anti-TL1A antibody comprises a heavy chain (HC) variable domain comprising SEQ ID NO: 271 and a light chain (LC) variable domain comprising SEQ ID NO: 280. In some cases, the anti-TL1A antibody comprises a heavy chain (HC) variable domain comprising SEQ ID NO: 271 and a light chain (LC) variable domain comprising SEQ ID NO: 281. In some cases, the anti-TL1A antibody comprises a heavy chain (HC) variable domain comprising SEQ ID NO: 271 and a light chain (LC) variable domain comprising SEQ ID NO: 282.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 275. In some cases, the anti-TL1A antibody comprises a heavy chain (HC) variable domain comprising SEQ ID NO: 272 and a light chain (LC) variable domain comprising SEQ ID NO: 276. In some cases, the anti-TL1A antibody comprises a heavy chain (HC) variable domain comprising SEQ ID NO: 272 and a light chain (LC) variable domain comprising SEQ ID NO: 277. In some cases, the anti-TL1A antibody comprises a heavy chain (HC) variable domain comprising SEQ ID NO: 272 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 279. In some cases, the anti-TL1A antibody comprises a heavy chain (HC) variable domain comprising SEQ ID NO: 272 and a light chain (LC) variable domain comprising SEQ ID NO: 280. In some cases, the anti-TL1A antibody comprises a heavy chain (HC) variable domain comprising SEQ ID NO: 272 and a light chain (LC) variable domain comprising SEQ ID NO: 281. In some cases, the anti-TL1A antibody comprises a heavy chain (HC) variable domain comprising SEQ ID NO: 272 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 273 and a light chain (LC) variable domain comprising SEQ ID NO: 275. In some cases, the anti-TL1A antibody comprises a heavy chain (HC) variable domain comprising SEQ ID NO: 273 and a light chain (LC) variable domain comprising SEQ ID NO: 276. In some cases, the anti-TL1A antibody comprises a heavy chain (HC) variable domain comprising SEQ ID NO: 273 and a light chain (LC) variable domain comprising SEQ ID NO: 277. In some cases, the anti-TL1A antibody comprises a heavy chain (HC) variable domain comprising SEQ ID NO: 273 and a light chain (LC) variable domain comprising SEQ ID NO: 278. In some cases, the anti-TL1A antibody comprises a heavy chain (HC) variable domain comprising SEQ ID NO: 273 and a light chain (LC) variable domain comprising SEQ ID NO: 279. In some cases, the anti-TL1A antibody comprises a heavy chain (HC) variable domain comprising SEQ ID NO: 273 and a light chain (LC) variable domain comprising SEQ ID NO: 280. In some cases, the anti-TL1A antibody comprises a heavy chain (HC) variable domain comprising SEQ ID NO: 273 and a light chain (LC) variable domain comprising SEQ ID NO: 281. In some cases, the anti-TL1A antibody comprises a heavy chain (HC) variable domain comprising SEQ ID NO: 273 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 274 and a light chain (LC) variable domain comprising SEQ ID NO: 279. In some cases, the anti-TL1A antibody comprises a heavy chain (HC) variable domain comprising SEQ ID NO: 274 and a light chain (LC) variable domain comprising SEQ ID NO: 280. In some cases, the anti-TL1A antibody comprises a heavy chain (HC) variable domain comprising SEQ ID NO: 274 and a light chain (LC) variable domain comprising SEQ ID NO: 281. In some cases, the anti-TL1A antibody comprises a heavy chain (HC) variable domain comprising SEQ ID NO: 274 and a light chain (LC) variable domain comprising SEQ ID NO: 282. In some cases, the anti-TL1A antibody comprises a heavy chain (HC) variable domain comprising SEQ ID NO: 274 and a light chain (LC) variable domain comprising SEQ ID NO: 275. In some cases, the anti-TL1A antibody comprises a heavy chain (HC) variable domain comprising SEQ ID NO: 274 and a light chain (LC) variable domain comprising SEQ ID NO: 276. In some cases, the anti-TL1A antibody comprises a heavy chain (HC) variable domain comprising SEQ ID NO: 274 and a light chain (LC) variable domain comprising SEQ ID NO: 277. In some cases, the anti-TL1A antibody comprises a heavy chain (HC) variable domain comprising SEQ ID NO: 274 and a light chain (LC) variable domain comprising SEQ ID NO: 278.

In some embodiments, the anti-TL1A antibody comprises a HCDR1 comprising SEQ ID NO: 283, a HCDR2 comprising SEQ ID NO: 284, a HCDR3 comprising SEQ ID NO: 285, a LCDR1 comprising SEQ ID NO: 286, a LCDR2 comprising SEQ ID NO: 287, and a LCDR3 comprising SEQ ID NO: 288. In some cases, the anti-TL1A antibody comprises a heavy chain (HC) variable domain comprising SEQ ID NO: 289 and a light chain (LC) variable domain comprising SEQ ID NO: 294. In some cases, the anti-TL1A antibody comprises a heavy chain (HC) variable domain comprising SEQ ID NO: 289 and a light chain (LC) variable domain comprising SEQ ID NO: 295. In some cases, the anti-TL1A antibody comprises a heavy chain (HC) variable domain comprising SEQ ID NO: 289 and a light chain (LC) variable domain comprising SEQ ID NO: 296. In some cases, the anti-TL1A antibody comprises a heavy chain (HC) variable domain comprising SEQ ID NO: 289 and a light chain (LC) variable domain comprising SEQ ID NO: 297. In some cases, the anti-TL1A antibody comprises a heavy chain (HC) variable domain comprising SEQ ID NO: 290 and a light chain (LC) variable domain comprising SEQ ID NO: 294. In some cases, the anti-TL1A antibody comprises a heavy chain (HC) variable domain comprising SEQ ID NO: 290 and a light chain (LC) variable domain comprising SEQ ID NO: 295. In some cases, the anti-TL1A antibody comprises a heavy chain (HC) variable domain comprising SEQ ID NO: 290 and a light chain (LC) variable domain comprising SEQ ID NO: 296. In some cases, the anti-TL1A antibody comprises a heavy chain (HC) variable domain comprising SEQ ID NO: 290 and a light chain (LC) variable domain comprising SEQ ID NO: 297. In some cases, the anti-TL1A antibody comprises a heavy chain (HC) variable domain comprising SEQ ID NO: 291 and a light chain (LC) variable domain comprising SEQ ID NO: 294. In some cases, the anti-TL1A antibody comprises a heavy chain (HC) variable domain comprising SEQ ID NO: 291 and a light chain (LC) variable domain comprising SEQ ID NO: 295. In some cases, the anti-TL1A antibody comprises a heavy chain (HC) variable domain comprising SEQ ID NO: 291 and a light chain (LC) variable domain comprising SEQ ID NO: 296. In some cases, the anti-TL1A antibody comprises a heavy chain (HC) variable domain comprising SEQ ID NO: 291 and a light chain (LC) variable domain comprising SEQ ID NO: 297. In some cases, the anti-TL1A antibody comprises a heavy chain (HC) variable domain comprising SEQ ID NO: 292 and a light chain (LC) variable domain comprising SEQ ID NO: 294. In some cases, the anti-TL1A antibody comprises a heavy chain (HC) variable domain comprising SEQ ID NO: 292 and a light chain (LC) variable domain comprising SEQ ID NO: 295. In some cases, the anti-TL1A antibody comprises a heavy chain (HC) variable domain comprising SEQ ID NO: 292 and a light chain (LC) variable domain comprising SEQ ID NO: 296. In some cases, the anti-TL1A antibody comprises a heavy chain (HC) variable domain comprising SEQ ID NO: 292 and a light chain (LC) variable domain comprising SEQ ID NO: 297. In some cases, the anti-TL1A antibody comprises a heavy chain (HC) variable domain comprising SEQ ID NO: 293 and a light chain (LC) variable domain comprising SEQ ID NO: 294. In some cases, the anti-TL1A antibody comprises a heavy chain (HC) variable domain comprising SEQ ID NO: 293 and a light chain (LC) variable domain comprising SEQ ID NO: 295. In some cases, the anti-TL1A antibody comprises a heavy chain (HC) variable domain comprising SEQ ID NO: 293 and a light chain (LC) variable domain comprising SEQ ID NO: 296. In some cases, the anti-TL1A antibody comprises a heavy chain (HC) variable domain comprising SEQ ID NO: 293 and a light chain (LC) variable domain comprising SEQ ID NO: 297.

In some embodiments, the anti-TL1A antibody comprises a HCDR1 comprising SEQ ID NO: 298, a HCDR2 comprising SEQ ID NO: 299, a HCDR3 comprising SEQ ID NO: 300, a LCDR1 comprising SEQ ID NO: 301, a LCDR2 comprising SEQ ID NO: 302, and a LCDR3 comprising SEQ ID NO: 303. In some cases, the anti-TL1A antibody comprises a heavy chain (HC) variable domain comprising SEQ ID NO: 304 and a light chain (LC) variable domain comprising SEQ ID NO: 305. In some cases, the anti-TL1A antibody comprises a heavy chain (HC) variable domain comprising SEQ ID NO: 306 and a light chain (LC) variable domain comprising SEQ ID NO: 307. In some cases, the anti-TL1A antibody comprises a heavy chain (HC) variable domain comprising SEQ ID NO: 308 and a light chain (LC) variable domain comprising SEQ ID NO: 309. In some cases, the anti-TL1A antibody comprises a heavy chain (HC) variable domain comprising SEQ ID NO: 310 and a light chain (LC) variable domain comprising SEQ ID NO: 311. In some cases, the anti-TL1A antibody comprises a heavy chain (HC) variable domain comprising SEQ ID NO: 312 and a light chain (LC) variable domain comprising SEQ ID NO: 313. In some cases, the anti-TL1A antibody comprises a heavy chain (HC) variable domain comprising SEQ ID NO: 314 and a light chain (LC) variable domain comprising SEQ ID NO: 315. In some cases, the anti-TL1A antibody comprises a heavy chain (HC) variable domain comprising SEQ ID NO: 316 and a light chain (LC) variable domain comprising SEQ ID NO: 317. In some cases, the anti-TL1A antibody comprises a heavy chain (HC) variable domain comprising SEQ ID NO: 318 and a light chain (LC) variable domain comprising SEQ ID NO: 319. In some cases, the anti-TL1A antibody comprises a heavy chain (HC) variable domain comprising SEQ ID NO: 320 and a light chain (LC) variable domain comprising SEQ ID NO: 321. In some cases, the anti-TL1A antibody comprises a heavy chain (HC) variable domain comprising SEQ ID NO: 322 and a light chain (LC) variable domain comprising SEQ ID NO: 323. In some cases, the anti-TL1A antibody comprises a heavy chain (HC) variable domain comprising SEQ ID NO: 324 and a light chain (LC) variable domain comprising SEQ ID NO: 325. In some cases, the anti-TL1A antibody comprises a heavy chain (HC) variable domain comprising SEQ ID NO: 326 and a light chain (LC) variable domain comprising SEQ ID NO: 327.

In some embodiments, the anti-TL1A antibody comprises a HCDR1 comprising SEQ ID NO: 328, a HCDR2 comprising SEQ ID NO: 329, a HCDR3 comprising SEQ ID NO: 330, a LCDR1 comprising SEQ ID NO: 331, a LCDR2 comprising SEQ ID NO: 332, and a LCDR3 comprising SEQ ID NO: 333. In some cases, the anti-TL1A antibody comprises a heavy chain (HC) variable domain comprising SEQ ID NO: 334 and a light chain (LC) variable domain comprising SEQ ID NO: 335.

In some embodiments, the anti-TL1A antibody comprises a HCDR1 comprising SEQ ID NO: 336, a HCDR2 comprising SEQ ID NO: 337, a HCDR3 comprising SEQ ID NO: 338, a LCDR1 comprising SEQ ID NO: 339, a LCDR2 comprising SEQ ID NO: 340, and a LCDR3 comprising SEQ ID NO: 341. In some cases, the anti-TL1A antibody comprises a heavy chain (HC) variable domain comprising SEQ ID NO: 342 and a light chain (LC) variable domain comprising SEQ ID NO: 343.

In some embodiments, the anti-TL1A antibody comprises a HCDR1 comprising SEQ ID NO: 346, a HCDR2 comprising SEQ ID NO: 347, a HCDR3 comprising SEQ ID NO: 348, a LCDR1 comprising SEQ ID NO: 349, a LCDR2 comprising SEQ ID NO: 350, and a LCDR3 comprising SEQ ID NO: 351. In some cases, the anti-TL1A antibody comprises a heavy chain (HC) variable domain comprising SEQ ID NO: 344 and a light chain (LC) variable domain comprising SEQ ID NO: 345. In some cases, the anti-TL1A antibody comprises a heavy chain (HC) variable domain comprising SEQ ID NO: 352 and a light chain (LC) variable domain comprising SEQ ID NO: 353. In some cases, the anti-TL1A antibody comprises a heavy chain (HC) variable domain comprising SEQ ID NO: 354 and a light chain (LC) variable domain comprising SEQ ID NO: 355. In some cases, the anti-TL1A antibody comprises a heavy chain (HC) variable domain comprising SEQ ID NO: 356 and a light chain (LC) variable domain comprising SEQ ID NO: 357.

In some embodiments, the anti-TL1A antibody comprises a HCDR1 comprising SEQ ID NO: 376, a HCDR2 comprising SEQ ID NO: 377, a HCDR3 comprising SEQ ID NO: 378, a LCDR1 comprising SEQ ID NO: 379, a LCDR2 comprising SEQ ID NO: 380, and a LCDR3 comprising SEQ ID NO: 381. In some cases, the anti-TL1A antibody comprises a heavy chain (HC) variable domain comprising SEQ ID NO: 382 and a light chain (LC) variable domain comprising SEQ ID NO: 383.

In some embodiments, the anti-TL1A antibody comprises a HCDR1 comprising SEQ ID NO: 384, a HCDR2 comprising SEQ ID NO: 385, a HCDR3 comprising SEQ ID NO: 386, a LCDR1 comprising SEQ ID NO: 387, a LCDR2 comprising SEQ ID NO: 388, and a LCDR3 comprising SEQ ID NO: 389. In some cases, the anti-TL1A antibody comprises a heavy chain (HC) variable domain comprising SEQ ID NO: 390 and a light chain (LC) variable domain comprising SEQ ID NO: 391. In some embodiments, the anti-TL1A antibody comprises one or more of A101-A124 of Table 19. In some embodiments, the anti-TL1A antibody is A100. In some embodiments, the anti-TL1A antibody is A101. In some embodiments, the anti-TL1A antibody is A102. In some embodiments, the anti-TL1A antibody is A103. In some embodiments, the anti-TL1A antibody is A104. In some embodiments, the anti-TL1A antibody is A105. In some embodiments, the anti-TL1A antibody is A106. In some embodiments, the anti-TL1A antibody is A107. In some embodiments, the anti-TL1A antibody is A108. In some embodiments, the anti-TL1A antibody is A109. In some embodiments, the anti-TL1A antibody is A110. In some embodiments, the anti-TL1A antibody is A111. In some embodiments, the anti-TL1A antibody is A112. In some embodiments, the anti-TL1A antibody is A113. In some embodiments, the anti-TL1A antibody is A114. In some embodiments, the anti-TL1A antibody is A115. In some embodiments, the anti-TL1A antibody is A116. In some embodiments, the anti-TL1A antibody is A117. In some embodiments, the anti-TL1A antibody is A118. In some embodiments, the anti-TL1A antibody is A119. In some embodiments, the anti-TL1A antibody is A120. In some embodiments, the anti-TL1A antibody is A121. In some embodiments, the anti-TL1A antibody is A122. In some embodiments, the anti-TL1A antibody is A123. In some embodiments, the anti-TL1A antibody is A124.

Micro-RNA miR-155 Modulators

Disclosed herein, in some embodiments, are therapeutic agents comprising modulators of miR-155 useful for the treatment of a disease or condition, or symptom of the disease or condition, disclosed herein. For example, the disease or condition is a PBmu subtype of Crohn's disease. In some embodiments, the therapeutic agents comprise a modulator of miR-155. In some cases, the modulator of miR-155 is an antagonist, partial antagonist, agonist, or partial agonist. In some embodiments, the miR-155 modulator modulates the expression of one or more genes comprising CSF, G-CSF, CM-CSF, M-CSF, Bcl211, Ccl2, Cd40, IL6, Nos2, Socsi, Stati, or Cxcr3, or a combination thereof. In some embodiments, the miR-155 modulator modulates the expression of one or more cytokines comprising IL-23/IL-17, GM-CSF, IL-6, IFNγ or TNF-α, or a combination thereof.

In some embodiments, the miR-155 modulator is a TNF-alpha receptor antagonist. In some embodiments, the miR-155 modulator is an anti-TNF-alpha antibody such as infliximab or adalimumab. In some embodiments, the miR-155 modulator is a TNF-alpha receptor, such as etanercept. In some embodiments, the miR-155 modulator is tenascin-c.

In certain embodiments, an miR-155 modulator comprises a molecule that upregulates expression of miR-155. In some embodiments, the miR-modulator is interferon-beta. In some embodiments, the miR-155 modulator is a toll-like receptor (TLR) ligand. In some embodiments, the TLR ligand is LPS, hypomethylated DNA, a TLR9 ligand, or PAm3CSK4.

In certain embodiments, an miR-155 modulator comprises a molecule that downregulates or otherwise inhibits miR-155. As a non-limiting example, the miR-155 modulator comprises Cobomarsen (MRG-106).

In some embodiments, the modulator of miR155 is an oligomer. In some embodiments, the modulator of miR-155 is a microRNA inhibitor. In some embodiments, the modulator of miR-155 is a microRNA mimic. In a non-limiting exemplary embodiment, the microRNA is microRNA-155 or a precursor thereof, such as a mammalian microRNA-155. Mammalian microRNA-155 includes human and mouse microRNA-155. In some embodiments, the miR-155 sequence comprises a sequence selected from SEQ ID NO 392-398 and SEQ ID NO: 405-408. In some embodiments, the miRNA mimic has the same sequence as a miRNA. In some embodiments, the miRNA is truncated. In some embodiments, the miRNA mimic is in the form of a double stranded molecule. In some embodiments, the miR-155 modulator comprises a sequence which is complementary to the seed sequence of the miR-155. In some embodiments, the seed sequence comprises a sequence selected from SEQ ID NO: 399-404.

In some embodiments, the oligonucleotide is 11, 12, 13, 14, 15, 16, 17, 18, 19, 20, 21, 22, 23, or 25 oligonucleotides long. In some embodiments, the oligonucleotide is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to a sequence contained in Table 3. In some embodiments, the miR-155 modulator comprises an antisense miR-155 oligonucleotide. In some embodiments, the antisense miR-155 oligonucleotide is complementary to a sequence found in Table 3. In some embodiments, the antisense miR-155 oligonucleotide is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to the naturally-occurring miRNA or the complement of the naturally occurring miRNA. In some embodiments, the miR-155 or anti-miR-155 oligonucleotide is modified with cholesterol. In some embodiments, the miRNA inhibitor comprises modified ribonucleotides. In some embodiments, the antisense miR-155 comprises a sequence complementary to a sequence found in Table 3.

TABLE 3
miR-155 and miR-155-derived sequences

| SEQ ID NO | Name | Sequence |
|---|---|---|
| 392 | miR-155 | UUAAUGCUAAUCGUGAUAGGGGU |
| 393 | miR-155 | GGGGAUAGUGCUAAUCGUAAUU |
| 394 | miR-155 | UAAUGCAUGGGGUGGGAGAGG |
| 395 | miR-155 | UAAUGCGUGGGGUGGGAGAGG |
| 396 | miR-155 | UUAAUGCUAA UCGUGAUAGG GG |
| 397 | miR-155-3p | CUCCUACAUAUUAGCAUUAACA |
| 398 | miR-155-5p | UUAAUGCUAAUCGUGAUAGGGGU |
| 399 | miR-155 seed | TAGCATTA |
| 400 | miR-155 seed | AGCATT |
| 401 | miR-155 seed | UAGCAUUAAC A |
| 402 | miR-155 seed | GCATTA |
| 403 | miR-155 seed | UAAUGCUA |
| 404 | miR-155 seed | AGCATTAA |
| 405 | Human-pre-miR-155 | CUGUUAAUGCUAAUCGUGAUAGGGGUUUUUGCCUC |
| | | CAACUGACUCCUACAUAUUAGCAUUAACAG |
| 406 | pre miR-155 | UUAAUGCUAA UCGUGAUAGG GGUUUUUGCC |
| | | UCCAACUGAC UCCUACAUAU |
| 407 | Mouse mature miR- | UUAAUGCUAAUUGUGAUAGGGGU |
| | 155 | |
| 408 | Mouse pre-miR-155 | CUGUUAAUGCUAAUUGUGAUAGGGGUUUUGGCCUC |
| | | UGACUGACUCCUACCUGUUAGCAUUAACAG |
| 409 | modified miR-155 | CCCCUAUCACGAUUAGCAUUAA |
| | targeting oligo | |

In some embodiments, the oligonucleotide may comprise at least one modified nucleotide. The modified nucleotide may comprise LNA. The modified nucleotide may be methylated. The modified nucleotide may comprise a sugar modification, such as a 2′-O-methlyation. The modified nucleotide may comprise a phosphorothioate linkage; 5-Methylcytosine; ethylene-bridged nucleotide (ENA); amino-2′-C-Bridged Bicyclic Nucleotide (CBBN) or a 2′flouro DNA nucleotide. The modified oligonucleotide may comprise an oligonucleotide listed in Table 4 or Table 5.

TABLE 4
Modified oligonucleotides. Capital Letters
without a superscript M or F, refer to LNA units.
Lower case = DNA, except for lower case in bold =
RNA. The LNA cytosines may optionally be
methylated). Capital letters followed by a
superscript M refer to 2′OME RNA units, Capital
letters followed by a superscript F refer to 2′
fluoro DNA units, lowercase letter refer to DNA

| Sequence | SEQ ID NO |
|---|---|
| 5′-CCCCtatcacgattagcaTTAA-3′ | 410 |
| 5′-cccctaTCACGATTagcattaa-3′ | 411 |
| 5′-cCccTatCacGatTagCatTaa-3′ | 412 |
| 5′-TcAcgATtaGcAtTA-3′ | 413 |
| 5′-TcAcGATtaGCAtTA-3′ | 414 |
| 5′-ACGATtAGCAtTA-3′ | 415 |
| 5′-GATtAGCaTTA-3′ | 416 |
| 5′-TC^{M}AC^{M}G^{M}ATTA^{M}GC^{M}AT^{M}TA-3′ | 417 |
| 5′-TC^{F}AC^{F}G^{F}ATT^{F}A^{F}GC^{F}AT^{F}TA-3′ | 418 |
| 5′-cCcCtAtCaCgAtTaGcAtTaa-3′ | 419 |
| 5′-tcAcgAttAgcAttAa-3′ | 420 |
| 5′-tCaCgAtTaGcAtTa-3′ | 421 |
| 5′-TcAcAATtaGCAtTA-3′ | 422 |
| 5′-TcAaCATtaGACtTA-3′ | 423 |
| 5′-TATGTAGGA-3′ | 424 |
| 5′-TTAGCATTA-3′ | 425 |
| 5′-TAGCATTA-3′ | 426 |
| 5′-AGCATTA-3′ | 427 |
| 5′-TATGTAGGA-3′ | 428 |
| 5′-ATGTAGGA-3′ | 429 |
| 5′-TGTAGGA-3′ | 430 |
| TaGCATTA | 431 |

TABLE 5
Modified oligo nucleotides that modulate miR-155.
¹l = locked nucleic acid modification; d = deoxyribonucleotide; s = phosphorothioate
linkage; md = 5-Methylcytosine; e = ethylene-bridged nucleotide (ENA); ab = amino-
2′-C-Bridged Bicyclic Nucleotide (CBBN).

| SEQ ID | |
|---|---|
| NO | Sequence |
| 432 | 5′-lAs.dTs.dCs.dAs.lCs.lGs.dAs.lTs.dTs.lAs.lGs.dCs.lAs.dTs.lTs.lA-3′ |
| 433 | 5′-lAs.dTs.dCs.dAs.lCs.lGs.dAs.dTs.lTs.lAs.lGs.dCs.lAs.dTs.lTs.lA-3′ |
| 434 | 5′-lAs.lTs.dCs.dAs.dCs.lGs.dAs.lTs.dTs.lAs.lGs.dCs.lAs.dTs.lTs.lA-3′ |
| 435 | 5′-lAs.lTs.dCs.dAs.dCs.lGs.lAs.dTs.dTs.lAs.lGs.lCs.dAs.lTs.dTs.lA-3′ |
| 436 | 5′-lAs.dTs.dCs.dAs.lCs.lGs.dAs.lTs.dTs.lAs.lGs.dCs.lAs.lTs.dTs.lA-3′ |
| 437 | 5′-lAs.lTs.dCs.dAs.lCs.dGs.dAs.dTs.lTs.lAs.dGs.lCs.lAs.dTs.lTs.lA-3 |
| 438 | 5-lAs.dTs.dCs.dAs.lCs.dGs.lAs.dTs.lTs.lAs.dGs.lCs.lAs.dTs.lTs.lA-3 |
| 439 | 5′-lAs.dTs.dCs.lAs.dCs.dGs.lAs.lTs.dTs.lAs.lGs.dCs.lAs.dTs.lTs.lA-3′ |
| 440 | 5′-lAs.dTs.lCs.dAs.dCs.lGs.dAs.lTs.lTs.dAs.dGs.lCs.lAs.dTs.lTs.lA-3′ |
| 441 | 5′-lAs.lTs.dCs.lAs.dCs.dGs.dAs.dTs.lTs.lAs.dGs.lCs.lAs.dTs.dTs.lA-3′ |
| 442 | 5′-lAs.dTs.lCs.dAs.dCs.dGs.lAs.dTs.lTs.lAs.dGs.lCs.lAs.dTs.lTs.lA-3′ |
| 443 | 5′-lAs.dTs.lCs.dAs.lCs.dGs.lAs.dTs.lTs.dAs.lGs.dCs.lAs.dTs.lTs.lA-3′ |
| 444 | 5′-lTs.dCs.dAs.lCs.dGs.dAs.lTs.dTs.dAs.lGs.dCs.lAs.lTs.dTs.lA-3′ |
| 445 | 5′-lTs.dCs.lAs.dCs.dGs.lAs.lTs.dTs.dAs.lGs.dCs.lAs.dTs.lTs.lA-3′ |
| 446 | 5′-lTs.dCs.dAs.dCs.lGs.lAs.lTs.dTs.dAs.lGs.dCs.lAs.dTs.lTs.lA-3′ |
| 447 | 5′-lTs.lCs.lAs.dCs.lGs.dAs.dTs.lTs.lAs.dGs.lCs.dAs.dTs.lTs.lA-3′ |
| 448 | 5′-lTs.dCs.dAs.lCs.dGs.dAs.dTs.lTs.lAs.lGs.lCs.lAs.lTs.lTs.lA-3′ |
| 449 | 5′-lTs.dCs.lAs.dCs.lGs.lAs.lTs.dTs.dAs.lGs.lCs.lAs.dTs.lTs.lA-3′ |
| 450 | 5′-lGs.lAs.lTs.lTs.lAs.lGs.dCs.lAs.lTs.dTs.lA-3′ |
| 451 | 5′-lCs.dGs.lAs.lTs.lTs.lAs.lGs.dCs.lAs.lTs.lTs.lA-3′ |
| 452 | 5′-lCs.dGs.lAs.lTs.lTs.dAs.lGs.dCs.lAs.lTs.lTs.lA-3′ |
| 453 | 5′-lCs.lAs.dCs.lGs.dAs.lTs.lTs.dAs.lGs.dCs.lAs.lTs.lTs.lA-3′ |
| 454 | 5′-lCs.dAs.lCs.dGs.dAs.lTs.lTs.dAs.lGs.dCs.lAs.lTs.lTs.lA-3′ |
| 455 | 5′-lTs.dCs.lAs.dCs.lGs.lAs.lTs.dTs.d.As.lGs.lCs.lAs.dTs.lTs.lA-3′ |
| 456 | 5′-lTs.lAs.lGs.lCs.lAs.lTs.lTs.lA-3′ |
| 457 | 5′-lCs.dAs.lCs.dGs.lAs.lTs.lTs.dAs.lGs.dCs.lAs.lTs.lTs.lA-3′ |
| 458 | 5′-lCs.dAs.lCs.dGs.lAs.dTs.lTs.dAs.lGs.dCs.lAs.lTs.lTs.lA-3′ |
| 459 | 5′-dCs.dAs.lCs.dGs.dAs.lTs.lTs.dAs.lGs.dCs.lAs.lTs.lTs.lA-3′ |
| 460 | 5′-lCs.dAs.lCs.dGs.dAs.lTs.lTs.lAs.lGs.dCs.lAs.dTs.lTs.lA-3′ |
| 461 | 5′-lCs.lAs.lCs.dGs.dAs.lTs.lTs.dAs.lGs.dCs.lAs.lTs.lTs.lA-3′ |
| 462 | 5′-lCs.dAs.dCs.dGs.dAs.lTs.lTs.dAs.lGs.dCs.lAs.lTs.lTs.lA-3′ |
| 463 | 5′-lCs.dAs.lCs.lGs.dAs.lTs.lTs.dAs.lGs.dCs.lAs.lTs.lTs.lA-3′ |
| 464 | 5′-lCs.dAs.lCs.dGs.lAs.lTs.lTs.dAs.lGs.dCs.lAs.lTs.lTs.lA-3′ |
| 465 | 5′-lCs.dAs.lCs.dGs.dAs.dTs.lTs.dAs.lGs.dCs.lAs.lTs.lTs.lA-3′ |
| 466 | 5′-lCs.dAs.lCs.dGs.dAs.lTs.dTs.dAs.lGs.dCs.lAs.lTs.lTs.lA-3′ |
| 467 | 5′-lCs.dAs.lCs.dGs.dAs.lTs.lTs.lAs.lGs.dCs.lAs.lTs.lTs.lA-3′ |
| 468 | 5′-lCs.dAs.lCs.dGs.dAs.lTs.lTs.dAs.dGs.dCs.lAs.lTs.lTs.lA-3′ |
| 469 | 5′-lCs.dAs.lCs.dGs.dAs.lTs.lTs.dAs.lGs.lCs.lAs.lTs.lTs.lA-3 |
| 470 | 5′-lCs.dAs.lCs.dGs.dAs.lTs.lTs.dAs.lGs.dCs.dAs.lTs.lTs.lA-3′ |
| 471 | 5′-lCs.dAs.lCs.dGs.dAs.lTs.lTs.dAs.lGs.dCs.lAs.dTs.lTs.lA-3′ |
| 472 | 5′-lCs.dAs.lCs.dGs.dAs.lTs.lTs.dAs.lGs.dCs.lAs.lTs.dTs.lA-3′ |
| 473 | 5′-lCs.dAs.lCs.dGs.dAs.lTs.lTs.dAs.lGs.dCs.lAs.lTs.lTs.dA-3′ |
| 474 | 5′-dCs.lAs.lCs.dGs.dAs.lTs.lTs.dAs.lGs.dCs.lAs.lTs.lTs.lA-3′ |
| 475 | 5′-lCs.lAs.dCs.dGs.dAs.lTs.lTs.dAs.lGs.dCs.lAs.lTs.lTs.lA-3′ |
| 476 | 5′-lCs.dAs.dCs.lGs.dAs.lTs.lTs.dAs.lGs.dCs.lAs.lTs.lTs.lA-3′ |
| 477 | 5′-lCs.dAs.lCs.dGs.JAs.dTs.lTs.dAs.lGs.dCs.lAs.lTs.lTs.lA-3′ |
| 478 | 5′-lCs.dAs.lCs.dGs.dAs.lTs.dTs.lAs.lGs.dCs.lAs.lTs.lTs.lA-3′ |
| 479 | 5′-lCs.dAs.lCs.dGs.dAs.lTs.lTs.lAs.dGs.dCs.lAs.lTs.lTs.lA-3′ |
| 480 | 5′-lCs.dAs.lCs.dGs.dAs.lTs.lTs.dAs.dGs.lCs.lAs.lTs.lTs.lA-3′ |
| 481 | 5′-lCs.dAs.lCs.dGs.dAs.lTs.lTs.dAs.dGs.lCs.lAs.lTs.lTs.lA-3′ |
| 482 | 5′-lAs.lCs.dGs.dAs.lTs.lTs.dAs.lGs.dCs.lAs.lTs.lTs.lA-3′ |
| 483 | 5′-lAs.lCs.dGs.dAs.lTs.lTs.dAs.lGs.dCs.lAs.lTs.lTs.lA-3′ |
| 484 | 5′-lCs.dGs.dAs.lTs.lTs.dAs.lGs.dCs.lAs.lTs.lTs.lA-3′ |
| 485 | 5′-dAs.lCs.dGs.dAs.lTs.lTs.dAs.lGs.dCs.lAs.lTs.lTs.lA-3′ |
| 486 | 5′-lAs.dCs.dGs.dAs.lTs.lTs.dAs.lGs.dCs.lAs.lTs.lTs.lA-3′ |
| 487 | 5′-lAs.lCs.lGs.dAs.lTs.lTs.dAs.lGs.dCs.lAs.lTs.lTs.lA-3′ |
| 488 | 5′-lAs.lCs.dGs.lAs.lTs.lTs.dAs.lGs.dCs.lAs.lTs.lTs.lA-3′ |
| 489 | 5′-lAs.lCs.dGs.dAs.dTs.lTs.dAs.lGs.dCs.lAs.lTs.lTs.lA-3′ |
| 490 | 5′-lAs.lCs.dGs.dAs.lTs.dTs.dAs.lGs.dCs.lAs.lTs.lTs.lA-3′ |
| 491 | 5′-lAs.lCs.dGs.dAs.lTs.lTs.lAs.lGs.dCs.lAs.lTs.lTs.lA-3′ |
| 492 | 5′-lAs.lCs.dGs.dAs.lTs.lTs.dAs.dGs.dCs.lAs.lTs.lTs.lA-3′ |
| 493 | 5′-lAs.lCs.dGs.dAs.lTs.lTs.dAs.lGs.lCs.lAs.lTs.lTs.lA-3′ |
| 494 | 5′-lAs.lCs.dGs.dAs.lTs.lTs.dAs.lGs.dCs.dAs.lTs.lTs.lA-3′ |
| 495 | 5′-lAs.lCs.dGs.dAs.lTs.lTs.dAs.lGs.dCs.lAs.dTs.lTs.lA-3′ |
| 496 | 5′-lAs.lCs.dGs.dAs.lTs.lTs.dAs.lGs.dCs.lAs.lTs.dTs.lA-3′ |
| 497 | 5′-lAs.lCs.dGs.dAs.lTs.lTs.dAs.lGs.dCs.lAs.lTs.dTs.lA-3′ |
| 498 | 5′-lAs.dCs.lGs.dAs.lTs.lTs.dAs.lGs.dCs.lAs.lTs.lTs.lA-3′ |
| 499 | 5′-lAs.lCs.dGs.lAs.dTs.lTs.dAs.lGs.dCs.lAs.lTs.lTs.lA-3′ |
| 500 | 5′-lAs.lCs.dGs.dAs.lTs.lTs.dAs.lGs.dCs.lAs.lTs.lTs.lA-3′ |
| 501 | 5′-lAs.lCs.dGs.dAs.lTs.dTs.lAs.lGs.dCs.lAs.lTs.lTs.lA-3′ |
| 502 | 5′-lAs.lCs.dGs.dAs.lTs.lTs.lAs.dGs.dCs.lAs.lTs.lTs.lA-3′ |
| 503 | 5′-lAs.lCs.dGs.dAs.lTs.lTs.dAs.dGs.lCs.lAs.lTs.lTs.lA-3′ |
| 504 | 5′-lAs.lCs.dGs.dAs.lTs.lTs.dAs.lGs.lCs.dAs.lTs.lTs.lA-3′ |
| 505 | 5′-dCs.dGs.dAs.lTs.lTs.dAs.lGs.dCs.lAs.lTs.lTs.lA-3′ |
| 506 | 5′-lCs.lGs.dAs.lTs.lTs.dAs.lGs.dCs.lAs.lTs.lTs.lA-3′ |
| 507 | 5′-lCs.dGs.lAs.lTs.lTs.dAs.lGs.dCs.lAs.lTs.lTs.lA-3′ |
| 508 | 5′-lCs.dGs.dAs.dTs.lTs.dAs.lGs.dCs.lAs.lTs.lTs.lA-3′ |
| 509 | 5′-lCs.dGs.dAs.lTs.dTs.dAs.lGs.dCs.lAs.lTs.lTs.lA-3′ |
| 510 | 5′-lCs.dGs.dAs.lTs.lTs.lAs.lGs.dCs.lAs.lTs.lTs.lA-3′ |
| 511 | 5′-lCs.dGs.dAs.lTs.lTs.dAs.dGs.dCs.lAs.lTs.lTs.lA-3′ |
| 512 | 5′-lCs.dGs.dAs.lTs.lTs.dAs.lGs.lCs.lAs.lTs.lTs.lA-3′ |
| 513 | 5′-lCs.dGs.dAs.lTs.lTs.dAs.lGs.dCs.dAs.lTs.lTs.lA-3′ |
| 514 | 5′-lCs.dGs.dAs.lTs.lTs.dAs.lGs.dCs.lAs.dTs.lTs.lA-3′ |
| 515 | 5′-lCs.dGs.dAs.lTs.lTs.dAs.lGs.dCs.lAs.lTs.dTs.lA-3′ |
| 516 | 5′-lCs.dGs.dAs.lTs.lTs.dAs.lGs.dCs.lAs.lTs.lTs.dA-3′ |
| 517 | 5′-dCs.lGs.dAs.lTs.lTs.dAs.lGs.dCs.lAs.lTs.lTs.lA-3 |
| 518 | 5′-lCs.dGs.lAs.dTs.lTs.dAs.lGs.dCs.lAs.lTs.lTs.lA-3′ |
| 519 | 5′-lCs.dGs.dAs.lTs.dTs.lAs.lGs.dCs.lAs.lTs.lTs.lA-3′ |
| 520 | 5′-lCs.dGs.dAs.lTs.lTs.lAs.dGs.dCs.lAs.lTs.lTs.lA-3′ |
| 521 | 5′-lCs.dGs.dAs.lTs.lTs.dAs.dGs.lCs.lAs.lTs.lTs.lA-3′ |
| 522 | 5′-lCs.dGs.dAs.lTs.lTs.dAs.lGs.lCs.dAs.lTs.lTs.lA-3′ |
| 523 | 5′-dGs.dAs.lTs.lTs.dAs.lGs.dCs.lAs.lTs.lTs.lA-3′ |
| 524 | 5′-lGs.lAs.lTs.lTs.dAs.lGs.dCs.lAs.lTs.lTs.lA-3′ |
| 525 | 5′-lGs.dAs.dTs.lTs.dAs.lGs.dCs.lAs.lTs.lTs.lA-3′ |
| 526 | 5′-lGs.dAs.lTs.dTs.dAs.lGs.dCs.lAs.lTs.lTs.lA-3′ |
| 527 | 5′-lGs.dAs.lTs.lTs.lAs.lGs.dCs.lAs.lTs.lTs.lA-3′ |
| 528 | 5′-lGs.dAs.lTs.lTs.dAs.dGs.dCs.lAs.lTs.lTs.lA-3′ |
| 529 | 5′-lGs.dAs.lTs.lTs.dAs.lGs.lCs.lAs.lTs.lTs.lA-3′ |
| 530 | 5′-lGs.dAs.lTs.lTs.dAs.lGs.dCs.dAs.lTs.lTs.lA-3′ |
| 531 | 5′-lGs.dAs.lTs.lTs.dAs.lGs.dCs.lAs.dTs.lTs.lA-3′ |
| 532 | 5′-lGs.dAs.lTs.lTs.dAs.lGs.dCs.lAs.lTs.dTs.lA-3′ |
| 533 | 5′-lGs.dAs.lTs.lTs.dAs.lGs.dCs.lAs.lTs.lTs.dA-3′ |
| 534 | 5′-dGs.lAs.lTs.lTs.dAs.lGs.dCs.lAs.lTs.lTs.dA-3′ |
| 535 | 5′-lGs.lAs.dTs.lTs.dAs.lGs.dCs.lAs.lTs.lTs.lA-3′ |
| 536 | 5′-lGs.dAs.lTs.dTs.lAs.lGs.dCs.lAs.lTs.lTs.lA-3′ |
| 537 | 5′-lGs.dAs.lTs.lTs.lAs.dGs.dCs.lAs.lTs.lTs.lA-3′ |
| 538 | 5′-lGs.dAs.lTs.lTs.dAs.dGs.lCs.lAs.lTs.lTs.lA-3′ |
| 539 | 5′-lGs.dAs.lTs.lTs.dAs.lGs.lCs.dAs.lTs.lTs.lA-3′ |
| 540 | 5′-eCs.dAs.eCs.dGs.dAs.eTs.eTs.dAs.eGs.dCs.eAs.eTs.eTs.eA-3′ |
| 541 | 5′-lCs.dAs.lCs.dGs.dAs.lTs.lTs.dAs.lGs.dCs.eAs.lTs.lTs.eA-3′ |
| 542 | 5′-eCs.dAs.eCs.dGs.dAs.lTs.lTs.dAs.lGs.dCs.lAs.lTs.lTs.lA-3′ |
| 543 | 5′-lCs.dAs.lCs.dGs.dAs.lTs.lTs.dAs.eGs.dCs.lAs.lTs.lTs.lA-3′ |
| 544 | 5′-lCs.dAs.lCs.dGs.dAs.eTs.eTs.dAs.lGs.dCs.lAs.eTs.eTs.lA-3′ |
| 545 | 5′-lCs.dAs.lCs.dGs.dAs.lTs.eTs.dAs.lGs.dCs.lAs.lTs.lTs.lA-3′ |
| 546 | 5′-lCs.dAs.lCs.dGs.dAs.lTs.lTs.dAs.lGs.dCs.lAs.lTs.lTs.lA-3′ |
| 547 | 5′-lCs.dAs.lCs.dGs.dAs.lTs.lTs.dAs.lGs.dCs.abAs.lTs.lTs.abA-3′ |
| 548 | 5′-abCs.d.As.abCs.dGs.dAs.abTs.abTs.dAs.abGs.dCs.abAs.abTs.abTs.abA-3′ |

In some embodiments, the miR-155 modulator is a guanylate cyclase C agonist or a guanylate cyclase C receptor agonist (GCRA). In some embodiments, the agonist is a GCRA peptide. In some embodiments, the GCRA peptides are analogues of plecanatide, uroguanylin, guanylin, lymphoguanylin and ST peptides. In some embodiments, the miR-155 modulator is plecanatide (SP-304), SP-333, or SP373. In some embodiments, the miR-155 modulator is a guanylate cyclase C agonist or a GCRA listed in Tables 6-12.

TABLE 6
Guanylate cyclase C receptor agonist peptides

| | | | SEQ |
|---|---|---|---|
| | Position of | | ID |
| Name | Disulfide bonds | Structure | NO |
| SP-304 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 549 |
| SP-326 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴-Leu¹⁵ | 550 |
| SP-327 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴ | 551 |
| SP-328 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³-Leu¹⁴ | 552 |
| SP-329 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³ | 553 |
| SP-330 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹²-Leu¹³ | 554 |
| SP-331 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹² | 555 |
| SP332 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 556 |
| SP-333 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 557 |
| SP-334 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 558 |
| SP-335 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 559 |
| SP-336 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 560 |
| SP-337 | C4:C12, C7:C15 | dAsn¹-Asp²-Glu³-Cys⁴-Glu⁵-dLeu⁶-Cys⁷-Val⁸-Asn⁹-Val¹⁰-Ala¹¹-Cys¹²-Thr¹³-Gly¹⁴-Cys¹⁵-dLeu¹⁶ | 561 |
| SP-338 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵ | 562 |
| SP-342 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 563 |
| SP-343 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 564 |
| SP-344 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 565 |
| SP-347 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 566 |
| SP-348 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 567 |
| SP-350 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 568 |
| SP-352 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 569 |
| SP-358 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶- | 570 |
| | | PEG3 | |
| SP-359 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 571 |
| SP-360 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 572 |
| SP-361 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 573 |
| SP-362 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 574 |
| SP-368 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dNal¹⁶ | 575 |
| SP-369 | C4:C12, C7:C15 | dAsn¹-Asp²-Glu³-Cys⁴-Glu⁵-Leu⁶-Cys⁷-AIB⁸-Asn⁹-AIB¹⁰-Ala¹¹-Cys¹²-Thr¹³-Gly¹⁴-Cys¹⁵-dLeu¹⁶ | 576 |
| SP-370 | C4:C12, 7:15 | dAsn¹-Asp²-Glu³-Cys⁴-Glu⁵-Leu⁶-Asp[Lactam]⁷-Val⁸-Asn⁹-Val¹⁰-Ala¹¹-Cys¹²-Thr¹³-Gly¹⁴-Orn¹⁵-dLeu¹ | 577 |
| SP-371 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 578 |
| SP-372 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 579 |
| N1 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 580 |
| N2 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 581 |
| N3 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 582 |
| N4 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 583 |
| N5 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 584 |
| N6 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 585 |
| N7 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 586 |
| N8 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 587 |
| N9 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 588 |
| N10 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 589 |
| N11 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 590 |
| N12 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 591 |
| N13 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 592 |
| Formula I | C4:C12, C7:C15 | Asn¹-Asp²-Glu³-Cys⁴-Xaa⁵-Xaa⁶-Cys⁷-Xaa⁸-Xaa⁹-Xaa¹⁰-Aaa¹¹-Cys¹²-Aaa¹³-Xaa¹⁴-Cys¹⁵-Xaa¹⁶ | 593 |
| Formula II | C4:C12, C7:C15 | Xaaₙ₁-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaaₙ₂ | 594 |
| Formula | 4:12, 7:15 | Xaaₙ₁-Maa⁴-Glu⁵-Xaa⁶-Maa⁷-Val⁸-Asn⁹-Val¹⁰-Ala¹¹-Maa¹²-Thr¹³-Gly¹⁴-Maa¹⁵-Xaaₙ₂ | 595 |
| III | | | |
| Formula | 4:12, 7:15 | Xaaₙ₁-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaaₙ₂ | 596 |
| IV | | | |
| Formula V | C4:C12, C7:C15 | Asn¹-Asp²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-Xaa¹⁶ | 597 |
| Formula | C4:C12, C7:C15 | dAsn¹-Glu²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 598 |
| Vi | | | |
| Formula | C4:C12, C7:C15 | dAsn¹-dGlu²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 699 |
| VII-a | | | |
| Formula | C4:C12, C7:C15 | dAsn¹-dAsp²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 600 |
| VII-b | | | |
| Formula | C4:C12, C7:C15 | dAsn¹-dAsp²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 601 |
| VIII | | | |
| Formula | C4:C12, C7:C15 | dAsn¹-dGlu²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 602 |
| IX | | | |
| Formula | C4:C12, C7:C15 | Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ | 603 |
| XXI | | | |

TABLE 7
Linaclotide and Derivatives

| | Position of | | SEQ ID |
|---|---|---|---|
| Name | Disulfide Bonds | Structure | NO: |
| SP-339 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 604 |
| (linaclotide) | | | |
| SP-340 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 605 |
| SP-349 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴- | 606 |
| | | PEG3 | |
| SP-353 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 607 |
| | | Tyr¹⁶ | |
| SP-354 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 608 |
| | | Tyr¹⁶ | |
| SP-355 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-dTyr¹⁴ | 609 |
| SP-357 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 610 |
| SP-374 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 611 |
| | | Tyr¹⁶ | |
| SP-375 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 612 |
| | | dTyr¹⁶ | |
| SP-376 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 613 |
| | | Tyr¹⁶ | |
| SP-377 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 614 |
| | | dTyr¹⁶ | |
| SP-378 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 615 |
| | | dTyr¹⁶ | |
| SP-379 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 616 |
| | | Tyr¹⁶ | |
| SP-380 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 617 |
| | | dTyr¹⁶ | |
| SP-381 | C3:C8, C4:C12, C7:15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 618 |
| | | dTyr¹⁶ | |
| SP-382 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 619 |
| | | Tyr¹⁶ | |
| SP-383 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 620 |
| | | dTyr¹⁶ | |
| SP384 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴-PEG3 | 621 |
| N14 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 622 |
| N15 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 623 |
| N16 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 624 |
| N17 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 625 |
| | | Cys¹⁵-Tyr¹⁶-PEG3 | |
| N18 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 626 |
| | | Cys¹⁵-Tyr¹⁶ | |
| N19 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 627 |
| | | Tyr¹⁶-PEG3 | |
| N20 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 628 |
| | | Cys¹⁵-Tyr¹⁶-PEG3 | |
| N21 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 629 |
| | | Cys¹⁵-Tyr¹⁶ | |
| N22 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 630 |
| | | Tyr¹⁶-PEG3 | |
| N23 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 631 |
| | | Cys¹⁵-Tyr¹⁶-PEG3 | |
| N24 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 632 |
| | | Cys¹⁵-Tyr¹⁶ | |
| N25 | C3:C8, C4:C12, C7:C15 | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵- | 633 |
| | | Tyr¹⁶-PEG3 | |
| N26 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³-Tyr¹⁴ | 634 |
| N27 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³-Tyr¹⁴ | 635 |
| N28 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³- | 636 |
| N29 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³ | 637 |
| N30 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³-Tyr¹⁴ | 638 |
| N31 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³ | 639 |
| Formula X | C9:C14, C10:C18, | Xaa¹-Xaa²-Xaa³-Xaa⁴-Xaa⁵-Xaa⁶-Asn⁷-Tyr⁸-Cys⁹-Cys¹⁰-Xaa¹¹-Tyr¹²-Cys¹³-Cys¹⁴-Xaa¹⁵- | 640 |
| | C13:C21 | Xaa¹⁶-Xaa¹⁷-Cys¹⁸-Xaa¹⁹-Xaa²⁰-Cys²¹-Xaa²² | |
| Formula XI | C9:C14, C10:C18, | Xaa¹-Xaa²-Xaa³-Xaa⁴-Xaa⁵-Xaa⁶-Asn⁷-Phe⁸-Cys⁹-Cys¹⁰-Xaa¹¹-Phe¹²-Cys¹³-Cys¹⁴-Xaa¹⁵- | 641 |
| | C13:C21 | Xaa¹⁶-Xaa¹⁷-Cys¹⁸-Xaa¹⁹-Xaa²⁰-Cys²¹-Xaa²² | |
| Formula XII | C3:C8, C4:C12, C7:C15 | Asn¹-Phe²-Cys³-Cys⁴-Xaa⁵-Phe⁶-Cys⁷-Cys⁸-Xaa⁹-Xaa¹⁰-Xaa¹¹-Cys¹²-Xaa¹³-Xaa¹⁴-Cys¹⁵- | 642 |
| | | Xaa¹⁶ | |
| Formula XIII | 3:8, 4:12, 7:15 | Asn¹-Phe²-Pen³-Cys⁴-Xaa⁵-Phe⁶-Cys⁷-Pen⁸-Xaa⁹-Xaa¹⁰-Xaa¹¹-Cys¹²-Xaa¹³-Xaa¹⁴-Cys¹⁵- | 643 |
| | | Xaa¹⁶ | |
| Formula XIV | 3:8, 4:12, 7:15 | Asn¹-Phe²-Maa³-Maa⁴-Xaa⁵-Xaa⁶-Maa⁷-Maa⁸-Xaa⁹-Xaa¹⁰-Xaa¹¹-Maa¹²-Xaa¹³-Xaa¹⁴- | 644 |
| | | Maa¹⁵-Xaa¹⁶ | |
| Formula XV | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³-Tyr¹⁴ | 645 |
| Formula XVI | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³ | 646 |
| Formula XVII | 1:6, 2:10, 5:13 | Xaaₙ₃-Maa¹-Maa²-Xaa³-Xaa⁴-Maa⁵-Maa⁶-Xaa⁷-Xaa⁸-Xaa⁹-Maa¹⁰-Xaa¹¹-Xaa¹²-Maa¹³- | 647 |
| | | Xaaₙ₂ | |

TABLE 8
GCRA Peptides

| | Position of | | SEQ ID |
|---|---|---|---|
| Name | Disulfide bonds | Structure | NO: |
| SP-363 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 648 |
| | | Gly¹⁴-Cys¹⁵-dLeu-AMIDE¹⁶ | |
| SP-364 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 649 |
| | | Gly¹⁴-Cys¹⁵-dSer¹⁶ | |
| SP-365 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 650 |
| | | Gly¹⁴-Cys¹⁵-dSer-AMIDE¹⁶ | |
| SP-366 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 651 |
| | | Gly¹⁴-Cys¹⁵-dTyr¹⁶ | |
| SP-367 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 652 |
| | | Gly¹⁴-Cys¹⁵-dTyr-AMIDE¹⁶ | |
| SP-373 | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 653 |
| | | Gly¹⁴-Cys¹⁵-dLeu-AMIDE¹⁶ | |
| | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 654 |
| | | Gly¹⁴-Cys¹⁵-Leu¹⁶ | |
| SP- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 655 |
| 304diPEG | | Gly¹⁴-Cys¹⁵-Leu¹⁶-PEG3 | |
| SP-304N- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 656 |
| PEG | | Gly¹⁴-Cys¹⁵-Leu¹⁶ | |
| SP-304C- | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴- | 657 |
| PEG | | Cys¹⁵-Leu¹⁶-PEG3 | |

TABLE 9
SP-304 Analogs, Uroguanylin, and Uroguanylin Analogs

| | Position of | | SEQ ID |
|---|---|---|---|
| Name | Disulfide bonds | Structure | NO |
| Formula | C4:C12, | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³- | 658 |
| XVIII | C7:C15 | Xaa¹⁴-Maa¹⁵-Xaa¹⁶ | |
| Uro- | C4:C12, | Asn¹-Asp²-Asp³-Cys⁴-Glu⁵-Leu⁶-Cys⁷-Val⁸-Asn⁹-Val¹⁰-Ala¹¹-Cys¹²-Thr¹³- | 659 |
| guanylin | C7:C15 | Gly¹⁴-Cys¹⁵-Leu¹⁶ | |
| N32 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 660 |
| | | Gly¹⁴-Cys¹⁵-Leu¹⁶ | |
| N33 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 661 |
| | | Gly¹⁴-Cys¹⁵-Leu¹⁶ | |
| N34 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 662 |
| | | Gly¹⁴-Cys¹⁵-Leu¹⁶ | |
| N35 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 663 |
| | | Gly¹⁴-Cys¹⁵-Leu¹⁶ | |
| N36 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³- | 664 |
| | | Gly¹⁴-Cys¹⁵-Leu¹⁶ | |
| N37 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 665 |
| | | Gly¹⁴-Cys¹⁵-Leu¹⁶ | |
| N38 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 666 |
| | | Gly¹⁴-Cys¹⁵-Leu¹⁶ | |
| N39 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 667 |
| | | Gly¹⁴-Cys¹⁵-Leu¹⁶ | |
| N40 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 668 |
| | | Gly¹⁴-Cys¹⁵-Leu¹⁶ | |
| N41 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 669 |
| | | Gly¹⁴-Cys¹⁵-Leu¹⁶ | |
| N42 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 670 |
| | | Gly¹⁴-Cys¹⁵-Leu¹⁶ | |
| N43 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 671 |
| | | Gly¹⁴-Cys¹⁵-Leu¹⁶ | |
| N44 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 672 |
| | | Gly¹⁴-Cys¹⁵-Leu¹⁶ | |
| N45 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 673 |
| | | Gly¹⁴-Cys¹⁵-Leu¹⁶ | |
| N46 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 674 |
| | | Gly¹⁴-Cys¹⁵-Leu¹⁶ | |
| N47 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 675 |
| | | Gly¹⁴-Cys¹⁵-Leu¹⁶ | |
| N48 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 676 |
| | | Gly¹⁴-Cys¹⁵-Leu¹⁶ | |
| N49 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 677 |
| | | Gly¹⁴-Cys¹⁵-Leu¹⁶ | |
| N50 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 678 |
| | | Gly¹⁴-Cys¹⁵-Leu¹⁶ | |
| N51 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 679 |
| | | Gly¹⁴-Cys¹⁵-Leu¹⁶ | |
| N52 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³-⁶ | 680 |
| | | Gly¹⁴-Cys¹⁵-Leu¹ | |
| N53 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 681 |
| | | Gly¹⁴-Cys¹⁵-Leu¹⁶ | |
| N54 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 682 |
| | | Gly¹⁴-Cys¹⁵-Leu¹⁶ | |
| N55 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 766 |
| | | Gly¹⁴-Cys¹⁵-Leu¹⁶ | |
| N56 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 683 |
| | | Gly¹⁴-Cys¹⁵-Leu¹⁶ | |
| N57 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 684 |
| | | Gly¹⁴-Cys¹⁵-Leu¹⁶ | |
| N58 | C4:C12, C7:C15 | Gln¹-Glu²-Asp³-Cys⁴-Glu⁵-Leu⁶-Cys⁷-Val⁸-Asn⁹-Val¹⁰-Ala¹¹-Cys¹²-Thr¹³- | 685 |
| | | Gly¹⁴-Cys¹⁵-Leu¹⁶ | |
| N59 | C4:C12, C7:C15 | Gln¹-Glu²-Glu³-Cys⁴-Glu⁵-Leu⁶-Cys⁷-Val⁸-Asn⁹-Val¹⁰-Ala¹¹-Cys¹²-Thr¹³- | 686 |
| | | Gly¹⁴-Cys¹⁵-Leu¹⁶ | |
| N60 | C4:C12, C7:C15 | Lys¹-Asp²-Asp³-Cys⁴-Glu⁵-Leu⁶-Cys⁷-Val⁸-Asn⁹-Val¹⁰-Ala¹¹-Cys¹²-Thr¹³- | 684 |
| | | Gly¹⁴-Cys¹⁵-Leu¹⁶ | |
| N61 | C4:C12, C7:C15 | Lys¹-Asp²-Glu³-Cys⁴-Glu⁵-Leu⁶-Cys⁷-Val⁸-Asn⁹-Val¹⁰-Ala¹¹-Cys¹²-Thr¹³- | 685 |
| | | Gly¹⁴-Cys¹⁵-Leu¹⁶ | |
| N62 | C4:C12, C7:C15 | Lys¹-Glu²-Asp³-Cys⁴-Glu⁵-Leu⁶-Cys⁷-Val⁸-Asn⁹-Val¹⁰-Ala¹¹-Cys¹²-Thr¹³- | 689 |
| | | Gly¹⁴-Cys¹⁵-Leu¹⁶ | |
| N63 | C4:C12, C7:C15 | Lys¹-Glu²-Glu³-Cys⁴-Glu⁵-Leu⁶-Cys⁷-Val⁸-Asn⁹-Val¹⁰-Ala¹¹-Cys¹²-Thr¹³- | 690 |
| | | Gly¹⁴-Cys¹⁵-Leu¹⁶ | |
| N65 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 691 |
| | | Gly¹⁴-Cys¹⁵-Leu¹⁶ | |
| N66 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 692 |
| | | Gly¹⁴-Cys¹⁵-Leu¹⁶ | |
| N67 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 693 |
| | | Gly¹⁴-Cys¹⁵-Leu¹⁶ | |
| N68 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 694 |
| | | Gly¹⁴-Cys¹⁵-Leu¹⁶ | |
| N69 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 695 |
| | | Gly¹⁴-Cys¹⁵-Leu¹⁶ | |
| N70 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 696 |
| | | Gly¹⁴-Cys¹⁵-Leu¹⁶ | |
| N71 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 697 |
| | | Gly¹⁴-Cys¹⁵-Leu¹⁶ | |
| N72 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 698 |
| | | Gly¹⁴-Cys¹⁵-Leu¹⁶ | |
| N73 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 699 |
| | | Gly¹⁴-Cys¹⁵-Leu¹⁶ | |
| N74 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 700 |
| | | Gly¹⁴-Cys¹⁵-Leu¹⁶ | |
| N75 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 701 |
| | | Gly¹⁴-Cys¹⁵-Leu¹⁶ | |
| N76 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 702 |
| | | Gly¹⁴-Cys¹⁵-Leu¹⁶ | |
| N77 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 703 |
| | | Gly¹⁴-Cys¹⁵-Leu¹⁶ | |
| N78 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 704 |
| | | Gly¹⁴-Cys¹⁵-Leu¹⁶ | |
| N79 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 705 |
| | | Gly¹⁴-Cys¹⁵-Leu¹⁶ | |
| N80 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 706 |
| | | Gly¹⁴-Cys¹⁵-Leu¹⁶ | |
| N81 | C4:C12, C7:C15 | Glu¹-Asp²-Asp³-Cys⁴-Glu⁵-Leu⁶-Cys⁷-Ile⁸-Asn⁹-Met¹⁰-Ala¹¹-Cys¹²-Thr¹³- | 707 |
| | | Gly¹⁴-Cys¹⁵-Leu¹⁶ | |
| N82 | C4:C12, C7:C15 | Glu¹-Asp²-Glu³-Cys⁴-Glu⁵-Leu⁶-Cys⁷-Ile⁸-Asn⁹-Met¹⁰-Ala¹¹-Cys¹²-Thr¹³- | 708 |
| | | Gly¹⁴-Cys¹⁵-Leu¹⁶ | |
| N83 | C4:C12, C7:C15 | Glu¹-Glu²-Asp³-Cys⁴-Glu⁵-Leu⁶-Cys⁷-Ile⁸-Asn⁹-Met¹⁰-Ala¹¹-Cys¹²-Thr¹³- | 709 |
| | | Gly¹⁴-Cys¹⁵-Leu¹⁶ | |
| N84 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 710 |
| | | Gly¹⁴-Cys¹⁵-Leu¹⁶ | |
| N85 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 711 |
| | | Gly¹⁴-Cys¹⁵-Leu¹⁶ | |
| N86 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 712 |
| | | Gly¹⁴-Cys¹⁵-Leu¹⁶ | |
| N87 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 713 |
| | | Gly¹⁴-Cys¹⁵-Leu¹⁶ | |
| N88 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 714 |
| | | Gly¹⁴-Cys¹⁵-Leu¹⁶ | |
| N89 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 715 |
| | | Gly¹⁴-Cys¹⁵-Leu¹⁶ | |
| N90 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 716 |
| | | Gly¹⁴-Cys¹⁵-Leu¹⁶ | |
| N91 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 717 |
| | | Gly¹⁴-Cys¹⁵-Leu¹⁶ | |
| N92 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 718 |
| | | Gly¹⁴-Cys¹⁵-Leu¹⁶ | |
| N93 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 719 |
| | | Gly¹⁴-Cys¹⁵-Leu¹⁶ | |
| N94 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 720 |
| | | Gly¹⁴-Cys¹⁵-Leu¹⁶ | |
| N95 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 721 |
| | | Gly¹⁴-Cys¹⁵-Leu¹⁶ | |
| N96 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 722 |
| | | Gly¹⁴-Cys¹⁵-Leu¹⁶ | |

TABLE 10
Guanylin and Analogs

| | Position of | | SEQ |
|---|---|---|---|
| | Disulfide | | ID |
| Name | bonds | Structure | NO |
| Formula | 4:12, 7:15 | Xaa¹-Xaa²-Xaa³-Maa⁴-Xaa⁵-Xaaa⁶-Maa⁷-Xaa⁸-Xaa⁹-Xaa¹⁰-Xaa¹¹-Maa¹²-Xaa¹³-Xaa¹⁴- | 723 |
| XIX | | Maa¹⁵ | |
| Guanylin | C4:C12, C7:C15 | Ser¹-His²-Thr³-Cys⁴-Glu⁵-Ile⁶-Cys⁷-Ala⁸-Phe⁹-Ala¹⁰-Ala¹¹-Cys¹²-Ala¹³-Gly¹⁴- | 724 |
| | | Cys¹⁵ | |
| Human | C4:C12, C7:C15 | Pro¹-Gly²-Thr³-Cys⁴-Glu⁵-Ile⁶-Cys⁷-Ala⁸-Tyr⁹-Ala¹⁰-Ala¹¹-Cys¹²-Thr¹³-Gly¹⁴- | 725 |
| Guanylin | | Cys¹⁵ | |
| N97 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 726 |
| N98 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 727 |
| N99 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 728 |
| N100 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 729 |
| N101 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 730 |
| N102 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 731 |
| N103 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 732 |
| N104 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 733 |
| N105 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 734 |
| N106 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 735 |
| N107 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 736 |
| N108 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 737 |
| N109 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 738 |
| N110 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 739 |
| N111 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 740 |
| N112 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 741 |
| N113 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 742 |
| N114 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 743 |
| N115 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 744 |
| N116 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 745 |
| N117 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 746 |
| N118 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 747 |
| N119 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 748 |
| N120 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 749 |
| N121 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 750 |
| N122 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 751 |
| N123 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 752 |
| N124 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 753 |
| N125 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴-Cys¹⁵ | 784 |
| N126 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴-Cys¹⁵ | 755 |
| N127 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴-Cys¹⁵ | 756 |
| N128 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴-Cys¹⁵ | 757 |

TABLE 11
Lymphoguanylin and Analogs

| | Position of | SEQ | |
|---|---|---|---|
| | Disulfide | ID | |
| Name | bonds | NO: | Structure |
| Formula | 4:12 | 767 | Xaa¹-Xaa²-Xaa³-Maa⁴-Xaa⁵-Xaa⁶-Maa⁷-Xaa⁸-Xaa⁹-xaa¹⁰-Xaa¹¹-Maa¹²-Xaa¹³-Xaa¹⁴-Xaaₙ₁¹⁵ |
| XX | | | |
| Lympho- | C4:C12 | 768 | Gln¹-Glu²-Glu³-Cys⁴-Glu⁵-Leu⁶-Cys⁷-Ile⁸-Asn⁹-Met¹⁰-Ala¹¹-Cys¹²-Thr¹³-Gly¹⁴-Tyr¹⁵ |
| guanylin | | | |
| N129 | C4:C12 | 769 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ |
| N130 | C4:C12 | 770 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ |
| N131 | C4:C12 | 771 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ |
| N132 | C4:C12 | 772 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ |
| N133 | C4:C12 | 773 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ |
| N134 | C4:C12 | 774 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ |
| N135 | C4:C12 | 775 | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ |
| N136 | C4:C12 | 776 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ |
| N137 | C4:C12 | 777 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ |
| N138 | C4:C12 | 778 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ |
| N139 | C4:C12 | 779 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ |
| N140 | C4:C12 | 780 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ |
| N141 | C4:C12 | 781 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ |
| N142 | C4:C12 | 782 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ |
| N143 | C4:C12 | 783 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ |
| N144 | C4:C12 | 784 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ |
| N145 | C4:C12, | 785 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N146 | C4:C12, | 786 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N147 | C4:C12, | 787 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N148 | C4:C12, | 788 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N149 | C4:C12, | 789 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N150 | C4:C12, | 790 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N151 | C4:C12, | 791 | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N152 | C4:C12, | 792 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N153 | C4:C12, | 793 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N154 | C4:C12, | 794 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N155 | C4:C12, | 795 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N156 | C4:C12, | 796 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N157 | C4:C12, | 797 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N158 | C4:C12, | 798 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N159 | C4:C12, | 799 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |
| N160 | C4:C12, | 800 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- |
| | C7:C15 | | Ser¹⁶ |

TABLE 12
ST Peptide and Analogues

| | Position of | | SEQ ID |
|---|---|---|---|
| Name | Disulfide bonds | Structure | NO |
| STPeptide | C9:C14, C10:C18, | Asn¹-Ser²-Ser³-Asn⁴-Ser⁵-Ser⁶-Asn⁷-Tyr⁸-Cys⁹-Cys¹⁰-Glu¹¹-Lys¹²-Cys¹³- | 758 |
| | C13:C21 | Cys¹⁴-Asn¹⁵-Pro¹⁶-Ala¹⁷-Cys¹⁸-Thr¹⁹-Gly²⁰-Cys²¹-Tyr²² | |
| N161 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 759 |
| | C7:C15 | Thr¹³-Gly¹⁴-Cys¹⁵-Tyr¹⁶-PEG3 | |
| N162 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 760 |
| | C7:C15 | Thr¹³-Gly¹⁴-Cys¹⁵-Tyr¹⁶ | |
| N163 | C3:C8, C4:C12, | Asn¹-Phe²-Cys³-Cys⁴-Glu⁵-Thr⁶-Cys⁷-Cys⁸-Asn⁹-Pro¹⁰-Ala¹¹-Cys¹²-Thr¹³- | 761 |
| | C7:C15 | Gly¹⁴-Cys¹⁵-Tyr¹⁶-PEG3 | |
| N164 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 762 |
| | C7:C15 | Gly¹⁴-Cys¹⁵-Tyr¹⁶ | |
| N165 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 763 |
| | C7:C15 | Gly¹⁴-Cys¹⁵-dTyr¹⁶ | |
| N166 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 764 |
| | C7:C15 | Gly¹⁴-Cys¹⁵-dTyr¹⁶ | |
| N167 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 765 |
| | C7:C15 | Gly¹⁴-Cys¹⁵-Tyr¹⁶ | |

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the additional therapeutic agent comprises an agonist or antagonist Janus Kinase 1 (JAK1). Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HCl, Tofacitinib (CP-690550, Tasocitinib), XL019, Pacritinib (SB1518), or ZM 39923 HCl.

Kinase Modulator Therapeutics

Non-limiting embodiments are provided herein wherein a therapeutic agent comprises a kinase modulator. In some embodiments, the kinase modulator is a therapeutic selected for and/or administered to a subject having a PBmu subtype of CD. Non-limiting exemplary kinases include PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1. Non-limiting examples of kinase targets include those in Table 20A. In some embodiments, a kinase target comprises one or more of the kinases of Table 20A. Non-limiting examples of kinase modulators includes those in Table 20B. In some embodiments, a kinase modulator comprises one or more kinase modulators of Table 20B.

In some embodiments, the kinase modulator modulates PDK1 (pyruvate dehydrogenase kinase 1). In some embodiments, the kinase modulator is an inhibitor of PDK1. Non-limiting exemplary kinase modulators for PDK1 include Celecoxib, 7-Hydroxystaurosporine, Bisindolylmaleimide VIII, Staurosporine, Dexfosfoserine, 10,11-dimethoxy-4-methyldibenzo[c,f]-2,7-naphthyridine-3,6-diamine; 5-hydroxy-3-[(1r)-1-(1h-pyrrol-2-yl)ethyl]-2h-indol-2-one; 1-{2-oxo-3-[(1r)-1-(1h-pyrrol-2-yl)ethyl]-2h-indol-5-yl}urea; 2-(1H-imidazol-1-yl)-9-methoxy-8-(2-methoxyethoxy)benzo[c][2,7]naphthyridin-4-amine; Bisindolylmaleimide I; 3-(1H-indol-3-yl)-4-(1-{2-[(2S)-1-methylpyrrolidinyl]ethyl}-1H-indol-3-yl)-1H-pyrrole-2,5-dione; 3-[1-(3-aminopropyl)-1h-indol-3-yl]-4-(1h-indol-3-yl)-1h-pyrrole-2,5-dione; Inositol 1,3,4,5-Tetrakisphosphate; Fostamatinib; and AR-12 (Arno Therapeutics).

In some embodiments, the kinase modulator modulates CDK11B (cyclin-dependent kinase 11B). In some embodiments, the kinase modulator is an inhibitor of CDK11B. Non-limiting exemplary kinase modulators for CDK11B include Phosphonothreonine, Alvocidib, SNS-032, and Seliciclib.

In some embodiments, the kinase modulator modulates ULK1 (Serine/threonine-protein kinase ULK1). In some embodiments, the kinase modulator is an inhibitor of ULK1. Non-limiting exemplary kinase modulators for ULK1 include Fostamatinib.

In some embodiments, the kinase modulator modulates RIPK1 (receptor-interacting serine/threonine-protein kinase 1). In some embodiments, the kinase modulator is an inhibitor of RIPK1. Non-limiting exemplary kinase modulators for RIPK1 include Fostamatinib.

In some embodiments, the kinase modulator modulates IKBKB (inhibitor of nuclear factor kappa-B kinase subunit beta). In some embodiments, the kinase modulator is an inhibitor of IKBKB. Non-limiting exemplary kinase modulators for IKBKB include Auranofin, Arsenic trioxide, MLN0415, Ertiprotafib, Sulfasalazine, Mesalazine, Acetylcysteine, Fostamatinib, and Acetylsalicylic acid.

In some embodiments, the kinase modulator modulates CDK9 (cyclin-dependent kinase 9). In some embodiments, the kinase modulator is an inhibitor of CDK9. Non-limiting exemplary kinase modulators for CDK9 include Riviciclib, Roniciclib, Seliciclib, Alvocidib, ATUVECICLIB, SNS-032 (BMS-387032), and AZD-5438 (AstraZeneca).

In some embodiments, the kinase modulator modulates STK11 (serine/threonine kinase 11). In some embodiments, the kinase modulator is an inhibitor of STK11. Non-limiting exemplary kinase modulators for STK11 include Metformin, magnesium, manganese, cyclic AMP, ATP, Midostaurin, Nintedanib, Ruboxistaurin, Sunitinib, and ADP.

In some embodiments, the kinase modulator modulates RAF1 (RAF proto-oncogene serine/threonine-protein kinase). In some embodiments, the kinase modulator is an inhibitor of RAF1. Non-limiting exemplary kinase modulators for RAF1 include Balamapimod, Dabrafenib, Regorafenib, Sorafenib, LErafAON, iCo-007, XL281, Cholecystokinin, and Fostamatinib.

In some embodiments, the kinase modulator modulates CSNK1A1 (Casein Kinase 1 Alpha 1). In some embodiments, the kinase modulator is an inhibitor of CSNK1A1. Non-limiting exemplary kinase modulators for CSNK1A1 include Fostamatinib, IC261, ATP, PF 670462, CKI 7 dihydrochloride, ADP, (R)-DRF053 dihydrochloride, D4476, LH846, PF 4800567 hydrochloride, PF 670462, CKI 7 dihydrochloride, IC261, Ruxolitinib, Bosutinib, Sorafenib, Sunitinib, and A-series of kinase inhibitors A14, A64, A47, A75, A51, and A86 (Cell. 2018 Sep. 20; 175(1): 171-185.e25).

In some embodiments, the kinase modulator modulates AURKB (Aurora kinase B). In some embodiments, the kinase modulator is an inhibitor of AURKB. Non-limiting exemplary kinase modulators for AURKB include Barasertib, Cenisertib, Danusertib, Ilorasertib, Tozasertib, Hesperidin, AT9283, Enzastaurin, Reversine, and Fostamatinib.

In some embodiments, the kinase modulator modulates ATR (serine/threonine-protein kinase ATR). In some embodiments, the kinase modulator is an inhibitor of ATR. Non-limiting exemplary kinase modulators for ATR include Ceralasertib, Berzosertib, diphenyl acetamidotrichloroethyl fluoronitrophenyl thiourea, BAY-1895344, and Nevanimibe hydrochloride.

In some embodiments, the kinase modulator modulates PRKAA2 (5′-AMP-activated protein kinase catalytic subunit alpha-2). In some embodiments, the kinase modulator is an inhibitor of PRKAA2. Non-limiting exemplary kinase modulators for PRKAA2 include Acetylsalicylic acid, Fostamatinib, Topiramate, and Adenosine phosphate.

In some embodiments, the kinase modulator modulates CHEK2 (checkpoint kinase 2). In some embodiments, the kinase modulator is an inhibitor of CHEK2. Non-limiting exemplary kinase modulators for CHEK2 include Prexasertib.

In some embodiments, the kinase modulator modulates PRKDC (DNA-dependent protein kinase catalytic subunit). In some embodiments, the kinase modulator is an inhibitor of PRKDC. Non-limiting exemplary kinase modulators for PRKDC include Wortmannin, Torin 2, PIK-75, peposertib, KU-0060648, AZD7648, NU-7441, PI-103, PP121, DNA-PK inhibitor III, NU-7026, DNA-PK inhibitor V, Trifluoperazine, Suramin, and Idelalisib.

In some embodiments, the kinase modulator modulates AURKA (Aurora Kinase A). In some embodiments, the kinase modulator is an inhibitor of AURKA. Non-limiting exemplary kinase modulators for AURKA include Alisertib, Cenisertib, Tozasertib, Danusertib, Ilorasertib, Phosphonothreonine, CYC116, AT9283, SNS-314, MLN8054, Enzastaurin, 4-(4-methylpiperazin-1-yl)-n-[5-(2-thienylacetyl)-1,5-dihydropyrrolo[3,4-c]pyrazol-3-yl]benzamide, AKI-001, 1-{5-[2-(thieno[3,2-d]pyrimidin-4-ylamino)ethyl]-1,3-thiazol-2-yl}-3-[3-(trifluoromethyl)phenyl]urea; 1-(5-{2-[(1-methyl-1H-pyrazolo[4,3-d]pyrimidin-7-yl)amino]ethyl}-1,3-thiazol-2-yl)-3-[3-(trifluoromethyl)phenyl]urea; N-{3-[(4-{[3-(trifluoromethyl)phenyl]amino}pyrimidin-2-yl)amino]phenyl}cyclopropanecarboxamide; N-butyl-3-{[6-(9H-purin-6-ylamino)hexanoyl]amino}benzamide; and Fostamatinib.

In some embodiments, the kinase modulator modulates RPS6KB1 (Ribosomal Protein S6 Kinase B1). In some embodiments, the kinase modulator is an inhibitor of RPS6KB1. Non-limiting exemplary kinase modulators for RPS6KB1 include LY2584702, PF-4708671, and GNE-3511.

In some embodiments, the kinase modulator modulates CSNK2A2 (Casein kinase II subunit alpha). In some embodiments, the kinase modulator is an inhibitor of CSNK2A2. Non-limiting exemplary kinase modulators for CSNK2A2 include Silmitasertib, [1-(6-{6-[(1-methylethyl)amino]-1H-indazol-1-yl}pyrazin-2-yl)-1H-pyrrol-3-yl]acetic acid, and Fostamatinib.

In some embodiments, the kinase modulator modulates PLK1 (Serine/threonine-protein kinase PLK1). In some embodiments, the kinase modulator is an inhibitor of PLK1. Non-limiting exemplary kinase modulators for PLK1 include Rigosertib, Volasertib, 3-[3-chloro-5-(5-{[(1S)-1-phenylethyl]amino}isoxazolo[5,4-c]pyridin-3-yl)phenyl]propan-1-ol; 3-[3-(3-methyl-6-{[(1S)-1-phenylethyl]amino}-1H-pyrazolo[4,3-c]pyridin-1-yl)phenyl]propenamide; 4-(4-methylpiperazin-1-yl)-n-[5-(2-thienylacetyl)-1,5-dihydropyrrolo[3,4-c]pyrazol-3-yl]benzamide; 1-[5-Methyl-2-(trifluoromethyl)furan-3-yl]-3-[5-[2-[[6-(1H-1,2,4-triazol-5-ylamino)pyrimidin-4-yl]amino]ethyl]-1,3-thiazol-2-yl]urea; Wortmannin, Fostamatinib, Onvansertib, HMN-214, Purpurogallin, BI-2536, GSK-461364, Tak-960, Volasertib trihydrochloride, Rigosertib sodium, and BI-2536 monohydrate.

In some embodiments, the kinase modulator modulates PRKAA1 (5′-AMP-activated protein kinase catalytic subunit alpha-1). In some embodiments, the kinase modulator is an inhibitor of PRKAA1. Non-limiting exemplary kinase modulators for PRKAA1 include Adenosine phosphate, ATP, Phenformin, and Fostamatinib.

In some embodiments, the kinase modulator modulates MTOR (Serine/threonine-protein kinase mTOR). In some embodiments, the kinase modulator is an inhibitor of MTOR. Non-limiting exemplary kinase modulators for MTOR include Vistusertib, Sapanisertib, Bimiralisib, Samotolisib, Panulisib, Omipalisib, Apitolisib, Voxtalisib, Dactolisib, Gedatolisib, SF1126, Rimiducid, XL765, Everolimus, Ridaforolimus, Temsirolimus, Sirolimus, Pimecrolimus, Fostamatinib, PKI-179, PF-04691502, GDC-0349, GSK-1059615, AZD-8055, CC-115, BGT-226, Sonolisib, MKC-1, Umirolimus, VS-5584, Onatasertib, Paxalisib, Bimiralisib, 2-Hydyroxyoleic acid, Ophiopogonin B, GNE-493, GNE-477, Guttiferone E, PF-04979064, Hypaphorine, Astragaloside II, PP-121, KU-0063794, PD-166866, PI-103, CGP-60474, AZD-1208, PP-242, AZD-1897, LY-294002, SF-1126, Licochalcone A, Puquitinib, Zotarolimus, Ridaforolimus, Tacrolimus, Voxtalisib hydrochloride, Bimiralisib hydrochloride, Bimiralisib hydrochloride monohydrate, Dactolisib tosylate, and Hypaphorine hydrochloride.

In some embodiments, the kinase modulator modulates CDK1 (cyclin-dependent kinase 1). In some embodiments, the kinase modulator is an inhibitor of CDK1. Non-limiting exemplary kinase modulators for CDK1 include Roniciclib, Riviciclib, Milciclib, Alsterpaullone, Alvocidib, Hymenialdisine, Indirubin-3′-monoxime, Olomoucine, SU9516, AT-7519, Seliciclib, Fostamatinib, OTX-008, and K-00546.

In some embodiments, the kinase modulator modulates CDK2 (cyclin-dependent kinase 2). In some embodiments, the kinase modulator is an inhibitor of CDK2. Non-limiting exemplary kinase modulators for CDK2 include Bosutinib, Roniciclib, Seliciclib, 4-[5-(Trans-4-Aminocyclohexylamino)-3-Isopropylpyrazolo[1,5-a]Pyrimidin-7-Ylamino]-N,N-Dimethylbenzenesulfonamide; Staurosporine; 4-(2,4-Dimethyl-Thiazol-5-Yl)-Pyrimidin-2-Ylamine; Olomoucine; 4-[(4-Imidazo[1,2-a]Pyridin-3-Ylpyrimidin-2-Yl)Amino]Benzenesulfonamide; 2-Amino-6-Chloropyrazine; 6-O-Cyclohexylmethyl Guanine; N-[4-(2-Methylimidazo[1,2-a]Pyridin-3-Yl)-2-Pyrimidinyl]Acetamide; 1-Amino-6-Cyclohex-3-Enylmethyloxypurine; N-(5-Cyclopropyl-1h-Pyrazol-3-Yl)Benzamide; Purvalanol; [4-(2-Amino-4-Methyl-Thiazol-5-Yl)-Pyrimidin-2-Yl]-(3-Nitro-Phenyl)-Amine; (5R)-5-{[(2-Amino-3H-purin-6-yl)oxy]methyl}-2-pyrrolidinone; 4-(2,4-Dimethyl-1,3-thiazol-5-yl)-N-[4-(trifluoromethyl)phenyl]-2-pyrimidinamine; Hymenialdisine; (5-Chloropyrazolo[1,5-a]Pyrimidin-7-Yl)-(4-Methanesulfonylphenyl)Amine; 4-(5-Bromo-2-Oxo-2h-Indol-3-Ylazo)-Benzenesulfonamide; 4-(2,5-Dichloro-Thiophen-3-Yl)-Pyrimidin-2-Ylamine; 4-[(6-Amino-4-Pyrimidinyl)Amino]Benzenesulfonamide; 4-[3-Hydroxyanilino]-6,7-Dimethoxyquinazoline; SU9516; 3-Pyridin-4-Yl-2,4-Dihydro-Indeno[1,2-.C]Pyrazole; (2E,3S)-3-hydroxy-5′-[(4-hydroxypiperidin-1-yl)sulfonyl]-3-methyl-1,3-dihydro-2,3′-biindol-2′(1′H)-one; 1-[(2-Amino-6,9-Dihydro-1h-Purin-6-Yl)Oxy]-3-Methyl-2-Butanol; 4-((3r,4s,5r)-4-Amino-3,5-Dihydroxy-Hex-1-Ynyl)-5-Fluoro-3-[1-(3-Methoxy-1h-Pyrrol-2-Yl)-Meth-(Z)-Ylidene]-1,3-Dihydro-Indol-2-One; Lysine Nz-Carboxylic Acid; [2-Amino-6-(2,6-Difluoro-Benzoyl)-Imidazo[1,2-a]Pyridin-3-Yl]-Phenyl-Methanone; N′-[4-(2,4-Dimethyl-1,3-thiazol-5-yl)-2-pyrimidinyl]-N-hydroxyimidoformamide; N′-(Pyrrolidino[2,1-B]Isoindolin-4-On-8-Yl)-N-(Pyridin-2-Yl)Urea; 2-[Trans-(4-Aminocyclohexyl)Amino]-6-(Benzyl-Amino)-9-Cyclopentylpurine; 4-[4-(4-Methyl-2-Methylamino-Thiazol-5-Yl)-Pyrimidin-2-Ylamino]-Phenol 3-[4-(2,4-Dimethyl-Thiazol-5-Yl)-Pyrimidin-2-Ylamino]-Phenol; phenylaminoimidazo(1,2-alpha)pyridine; Olomoucine II; Triazolopyrimidine; Alvocidib; Seliciclib; 4-[(7-oxo-7h-thiazolo[5,4-e]indol-8-ylmethyl)-amino]-n-pyridin-2-yl-benzene sulfonamide; (13R,15S)-13-methyl-16-oxa-8,9,12,22,24-pentaazahexacyclo[15.6.2.16,9.1,12,15.0,2,7.0,21,25]heptacosa-1(24),2,4,6,17(25),18,20-heptaene-23,26-dione; N-(3-cyclopropyl-1H-pyrazol-5-yl)-2-(2-naphthyl)acetamide; 2-anilino-6-cyclohexylmethoxypurine; 1-(5-OXO-2,3,5,9B-tetrahydro-1h-pyrrolo[2,1-a]isoindol-9-yl)-3-(5-pyrrolidin-2-yl-1h-pyrazol-3-yl)-urea; (5-phenyl-7-(pyridin-3-ylmethylamino)pyrazolo[1,5-a]pyrimidin-3-yl)methanol; 2-(3,4-dihydroxyphenyl)-8-(1,1-dioxidoisothiazolidin-2-yl)-3-hydroxy-6-methyl-4h-chromen-4-one; (2R)-1-(dimethylamino)-3-{4-[(6-{[2-fluoro-5-(trifluoromethyl)phenyl]amino}pyrimidin-4-yl)amino]phenoxy}propan-2-ol; 5-(2,3-dichlorophenyl)-N-(pyridin-4-ylmethyl)-3-thiocyanatopyrazolo[1,5-a]pyrimidin-7-amine; O6-cyclohexylmethoxy-2-(4′-sulphamoylanilino) purine; (2S)-N-[(3E)-5-Cyclopropyl-3H-pyrazol-3-ylidene]-2-[4-(2-oxo-1-imidazolidinyl)phenyl]propenamide; 5-[(2-aminoethyl)amino]-6-fluoro-3-(1h-pyrrol-2-yl)benzo[cd]indol-2(1h)-one; N-cyclopropyl-4-pyrazolo[1,5-b]pyridazin-3-ylpyrimidin-2-amine; 3-((3-bromo-5-o-tolylpyrazolo[1,5-a]pyrimidin-7-ylamino)methyl)pyridine 1-oxide; 6-cyclohexylmethoxy-2-(3′-chloroanilino) purine; 3-bromo-5-phenyl-N-(pyridin-4-ylmethyl)pyrazolo[1,5-a]pyrimidin-7-amine; N-[5-(1,1-dioxidoisothiazolidin-2-yl)-1h-indazol-3-yl]-2-(4-piperidin-1-ylphenyl)acetamide; (3R)-3-(aminomethyl)-9-methoxy-1,2,3,4-tetrahydro-5H-[1]benzothieno[3,2-e][1,4]diazepin-5-one; 5-[5,6-bis(methyloxy)-1h-benzimidazol-1-yl]-3-{[1-(2-chlorophenyl)ethyl]oxy}-2-thiophenecarboxamide; 5-Bromoindirubin; (2S)-1-{4-[(4-Anilino-5-bromo-2-pyrimidinyl)amino]phenoxy}-3-(dimethylamino)-2-propanol; (2R)-1-{4-[(4-Anilino-5-bromo-2-pyrimidinyl)amino]phenoxy}-3-(dimethylamino)-2-propanol; (5E)-2-Amino-5-(2-pyridinylmethylene)-1,3-thiazol-4(5H)-one; 4-{5-[(Z)-(2,4-dioxo-1,3-thiazolidin-5-ylidene)methyl]furan-2-yl}benzenesulfonamide; 4-{5-[(Z)-(2-imino-4-oxo-1,3-thiazolidin-5-ylidene)methyl]-2-furyl}-n-methylbenzene sulfonamide; 4-{5-[(Z)-(2-imino-4-oxo-1,3-thiazolidin-5-ylidene)methyl]furan-2-yl}benzenesulfonamide; 4-{5-[(Z)-(2-imino-4-oxo-1,3-thiazolidin-5-ylidene)methyl]furan-2-yl}-2-(trifluoromethyl)benzene sulfonamide; 4-{5-[(Z)-(2-imino-4-oxo-1,3-thiazolidin-5-ylidene)methyl]furan-2-yl}benzoic acid; 4-{5-[(1Z)-1-(2-imino-4-oxo-1,3-thiazolidin-5-ylidene)ethyl]-2-furyl}benzenesulfonamide; N-[4-(2,4-dimethyl-thiazol-5-yl)-pyrimidin-2-yl]-n′,n′-dimethyl-benzene-1,4-diamine; (5Z)-5-(3-bromocyclohexa-2,5-dien-1-ylidene)-n-(pyridin-4-ylmethyl)-1,5-dihydropyrazolo[1,5-a]pyrimidin-7-amine; 6-(3,4-dihydroxybenzyl)-3-ethyl-1-(2,4,6-trichlorophenyl)-1h-pyrazolo[3,4-d]pyrimidin-4(5h)-one; 6-(3-aminophenyl)-n-(tert-butyl)-2-(trifluoromethyl)quinazolin-4-amine; 2-(4-(aminomethyl)piperidin-1-yl)-n-(3_cyclohexyl-4-oxo-2,4-dihydroindeno[1,2-c]pyrazol-5-yl)acetamide; 1-(3-(2,4-dimethylthiazol-5-yl)-4-oxo-2,4-dihydroindeno[1,2-c]pyrazol-5-yl)-3-(4-methylpiperazin-1-yl)urea; 4-{[5-(cyclohexylmethoxy) [1,2,4]triazolo[1,5-a]pyrimidin-7-yl]amino}benzene sulfonamide; 4-{[5-(cyclohexylamino)[1,2,4]triazolo[1,5-a]pyrimidin-7-yl]amino}benzenesulfonamide; 4-({5-[(4-aminocyclohexy)amino][1,2,4]triazolo[1,5-a]pyrimidin-7-yl}amino)benzenesulfonamide; 4-{[5-(cyclohexyloxy)[1,2,4]triazolo[1,5-a]pyrimidin-7-yl]amino}benzene sulfonamide; CAN-508; (2R)-1-[4-({4-[(2,5-Dichlorophenyl)amino]-2-pyrimidinyl}amino)phenoxy]-3-(dimethylamino)-2-propanol; (2S)-1-[4-({6-[(2,6-Difluorophenyl)amino]-4-pyrimidinyl}amino)phenoxy]-3-(dimethylamino)-2-propanol; (2S)-1-[4-({4-[(2,5-Dichlorophenyl)amino]-2-pyrimidinyl}amino)phenoxy]-3-(dimethylamino)-2-propanol; (2R)-1-[4-({6-[(2,6-Difluorophenyl)amino]-4-pyrimidinyl}amino)phenoxy]-3-(dimethylamino)-2-propanol; N-(2-methoxyethyl)-4-({4-[2-methyl-1-(1-methylethyl)-1h-imidazol-5-yl]pyrimidin-2-yl}amino)benzene sulfonamide; 4-{[4-(1-cyclopropyl-2-methyl-1h-imidazol-5-yl)pyrimidin-2-yl]amino}-n-methylbenzene sulfonamide; 1-(3,5-dichlorophenyl)-5-methyl-1h-1,2,4-triazole-3-carboxylic acid; (2S)-1-(Dimethylamino)-3-(4-{[4-(2-methylimidazo[1,2-a]pyridin-3-yl)-2-pyrimidinyl]amino}phenoxy)-2-propanol; N-(4-{[(3S)-3-(dimethylamino)pyrrolidin-1-yl]carbonyl}phenyl)-5-fluoro-4-[2-methyl-1-(1-methylethyl)-1H-imidazol-5-yl]pyrimidin-2-amine; 2-{4-[4-({4-[2-methyl-1-(1-methylethyl)-1H-imidazol-5-yl]pyrimidin-2-yl}amino)phenyl]piperazin-1-yl}-2-oxoethanol; Indirubin-3′-monoxime; N-[3-(1H-benzimidazol-2-yl)-1h-pyrazol-4-yl]benzamide; RO-4584820; N-Methyl-4-{[(2-oxo-1,2-dihydro-3H-indol-3-ylidene)methyl]amino}benzenesulfonamide; N-methyl-{4-[2-(7-oxo-6,7-dihydro-8H-[1,3]thiazolo[5,4-e]indol-8-ylidene)hydrazino]phenyl}methanesulfonamide; 3-{[(2,2-dioxido-1,3-dihydro-2-benzothien-5-yl)amino]methylene}-5-(1,3-oxazol-5-yl)-1,3-dihydro-2H-indol-2-one; 4-{[(2-Oxo-1,2-dihydro-3H-indol-3-ylidene)methyl]amino}-N-(1,3-thiazol-2-yl)benzenesulfonamide; 3-{[4-([amino(imino)methyl]aminosulfonyl)anilino]methylene}-2-oxo-2,3-dihydro-1H-indole; 5-hydroxynaphthalene-1-sulfonamide; N-(4-sulfamoylphenyl)-1H-indazole-3-carboxamide 4-[(6-chloropyrazin-2-yl)amino]benzenesulfonamide; N-phenyl-1H-pyrazole-3-carboxamide; 4-(acetylamino)-N-(4-fluorophenyl)-1H-pyrazole-3-carboxamide; (4E)-N-(4-fluorophenyl)-4-[(phenylcarbonyl)imino]-4H-pyrazole-3-carboxamide; {[(2,6-difluorophenyl)carbonyl]amino}-N-(4-fluorophenyl)-1H-pyrazole-3-carboxamide; 5-chloro-7-[(1-methylethyl)amino]pyrazolo[1,5-a]pyrimidine-3-carbonitrile; 5-[4-(4-aminocyclohexyl)amino]-7-(propan-2-ylamino)pyrazolo[1,5-a]pyrimidine-3-carbonitrile; 4-{[(2,6-difluorophenyl)carbonyl]amino}-N-[(3S)-piperidin-3-yl]-1H-pyrazole-3-carboxamide; AT-7519; 4-(4-methoxy-1H-pyrrolo[2,3-b]pyridin-3-yl)pyrimidin-2-amine; 4-(4-propoxy-1H-pyrrolo[2,3-b]pyridin-3-yl)pyrimidin-2-amine; hydroxy(oxo)(3-{[(2z)-4-[3-(1h-1,2,4-triazol-1-ylmethyl)phenyl]pyrimidin-2(5h)-ylidene]amino}phenyl)ammonium; 4-Methyl-5-[(2Z)-2-{[4-(4-morpholinyl)phenyl]imino}-2,5-dihydro-4-pyrimidinyl]-1,3-thiazol-2-amine; 6-cyclohexylmethyloxy-2-(4′-hydroxyanilino)purine; 4-(6-cyclohexylmethoxy-9h-purin-2-ylamino)-benzamide; 6-(cyclohexylmethoxy)-8-isopropyl-9h-purin-2-amine; 3-(6-cyclohexylmethoxy-9h-purin-2-ylamino)-benzene sulfonamide; (2R)-2-{[4-(benzylamino)-8-(1-methylethyl)pyrazolo[1,5-a][1,3,5]triazin-2-yl]amino}butan-1-ol; 3-({2-[(4-{[6-(cyclohexylmethoxy)-9h-purin-2-yl]amino}phenyl)sulfonyl]ethyl}amino)propan-1-ol; 6-cyclohexylmethyloxy-5-nitroso-pyrimidine-2,4-diamine; 1-methyl-8-(phenylamino)-4,5-dihydro-1H-pyrazolo[4,3-h]quinazoline-3-carboxylic acid; 6-bromo-13-thia-2,4,8,12,19-pentaazatricyclo[12.3.1.1˜3,7˜]nonadeca-1(18),3(19),4,6,14,16-hexaene 13,13-dioxide; (2R)-2-({9-(1-methylethyl)-6-[(4-pyridin-2-ylbenzyl)amino]-9H-purin-2-yl}amino)butan-1-ol; 1-[4-(aminosulfonyl)phenyl]-1,6-dihydropyrazolo[3,4-e]indazole-3-carboxamide; 5-(2,3-dichlorophenyl)-N-(pyridin-4-ylmethyl)pyrazolo[1,5-a]pyrimidin-7-amine; 6-(2-fluorophenyl)-N-(pyridin-3-ylmethyl)imidazo[1,2-a]pyrazin-8-amine; 3-methyl-N-(pyridin-4-ylmethyl)imidazo[1,2-a]pyrazin-8-amine; 5-(2-fluorophenyl)-N-(pyridin-4-ylmethyl)pyrazolo[1,5-a]pyrimidin-7-amine; 3-bromo-5-phenyl-N-(pyridin-3-ylmethyl)pyrazolo[1,5-a]pyrimidin-7-amine; 3-bromo-5-phenyl-N-(pyrimidin-5-ylmethyl)pyrazolo[1,5-a]pyridin-7-amine; 3-bromo-6-phenyl-N-(pyrimidin-5-ylmethyl)imidazo[1,2-a]pyridin-8-amine; N-((2-aminopyrimidin-5-yl)methyl)-5-(2,6-difluorophenyl)-3-ethylpyrazolo[1,5-a]pyrimidin-7-amine; 3-cyclopropyl-5-phenyl-N-(pyridin-3-ylmethyl)pyrazolo[1,5-a]pyrimidin-7-amine; 4-{[4-amino-6-(cyclohexylmethoxy)-5-nitrosopyrimidin-2-yl]amino}benzamide; 4-[(5-isopropyl-1,3-thiazol-2-yl)amino]benzenesulfonamide; N-(5-Isopropyl-thiazol-2-YL)-2-pyridin-3-YL-acetamide; Variolin B; N(6)-dimethylallyladenine; Bosutinib, Milciclib, SNS-032, CVT-313, Isoindirubin, Amygdalin, Zotiraciclib citrate, Milciclib maleate, and Indirubin.

In some embodiments, the kinase modulator modulates MAPK1 (mitogen-activated protein kinase 1). In some embodiments, the kinase modulator is an inhibitor of MAPK1. Non-limiting exemplary kinase modulators for MAPK1 include Ulixertinib, Arsenic trioxide, Phosphonothreonine, Purvalanol, Seliciclib, Perifosine, Isoprenaline, N,N-dimethyl-4-(4-phenyl-1h-pyrazol-3-yl)-1h-pyrrole-2-carboxamide; N-benzyl-4-[4-(3-chlorophenyl)-1h-pyrazol-3-yl]-1h-pyrrole-2-carboxamide; (S)—N-(1-(3-chloro-4-fluorophenyl)-2-hydroxyethyl)-4-(4-(3-chlorophenyl)-1h-pyrazol-3-yl)-1h-pyrrole-2-carboxamide; (3R,5Z,8S,9S,11E)-8,9,16-trihydroxy-14-methoxy-3-methyl-3,4,9,10-tetrahydro-1h-2-benzoxacyclotetradecine-1,7(8h)-dione; 5-(2-phenylpyrazolo[1,5-a]pyridin-3-yl)-1h-pyrazolo[3,4-c]pyridazin-3-amine; (1aR,8S,13S,14S,15aR)-5,13,14-trihydroxy-3-methoxy-8-methyl-8,9,13,14,15,15a-hexahydro-6H-oxireno[k][2]benzoxacyclotetradecine-6,12(1aH)-dione; Olomoucine; [4-({5-(aminocarbonyl)-4-[(3-methylphenyl)amino]pyrimidin-2-yl}amino)phenyl]acetic acid; 4-[4-(4-fluorophenyl)-2-[4-[(r)-methylsulfinyl]phenyl]-1h-imidazol-5-yl]pyridine; SB220025; and Turpentine.

In some embodiments, the kinase modulator modulates GSK3B (Glycogen Synthase Kinase 3 Beta). In some embodiments, the kinase modulator is an inhibitor of GSK3B. Non-limiting exemplary kinase modulators for GSK3B include Lithium cation; 3-[3-(2,3-Dihydroxy-Propylamino)-Phenyl]-4-(5-Fluoro-1-Methyl-1h-Indol-3-Yl)-Pyrrole-2,5-Dione; SB-409513; AR-AO-14418; Staurosporine; Indirubin-3′-monoxime; Alsterpaullone; Phosphoaminophosphonic Acid-Adenylate Ester; 2-(1,3-benzodioxol-5-yl)-5-[(3-fluoro-4-methoxybenzyl)sulfanyl]-1,3,4-oxadiazole; 5-[1-(4-methoxyphenyl)-1H-benzimidazol-6-yl]-1,3,4-oxadiazole-2(3H)-thione; (7S)-2-(2-aminopyrimidin-4-yl)-7-(2-fluoroethyl)-1,5,6,7-tetrahydro-4H-pyrrolo[3,2-c]pyridin-4-one; 6-bromoindirubin-3′-oxime; N-[2-(5-methyl-4H-1,2,4-triazol-3-yl)phenyl]-7H-pyrrolo[2,3-d]pyrimidin-4-amine; 5-(5-chloro-7H-pyrrolo[2,3-d]pyrimidin-4-yl)-4,5,6,7-tetrahydro-1H-imidazo[4,5-c]pyridine; 3-({[(3S)-3,4-dihydroxybutyl]oxy}amino)-1H,2′H-2,3′-biindol-2′-one; N-[(1S)-2-amino-1-phenylethyl]-5-(1H-pyrrolo[2,3-b]pyridin-4-yl)thiophene-2-carboxamide; 4-(4-chlorophenyl)-4-[4-(1h-pyrazol-4-yl)phenyl]piperidine; isoquinoline-5-sulfonic acid (2-(2-(4-chlorobenzyloxy)ethylamino)ethyl)amide; (2S)-1-(1H-indol-3-yl)-3-{[5-(3-methyl-1h-indazol-5-yl)pyridin-3-yl]oxy}propan-2-amine; Tideglusib; Fostamatinib; Lithium citrate; Lithium succinate; and Lithium carbonate.

In some embodiments, the kinase modulator modulates CSNK2A1 (Casein kinase II subunit alpha). In some embodiments, the kinase modulator is an inhibitor of CSNK2A1. Non-limiting exemplary kinase modulators for CSNK2A1 include Silmitasertib, Benzamidine; Phosphoaminophosphonic Acid-Adenylate Ester; Tetrabromo-2-Benzotriazole; Resveratrol; s-methyl-4,5,6,7-tetrabromo-benzimidazole; Emodin; 3,8-dibromo-7-hydroxy-4-methyl-2h-chromen-2-one; 1,8-Di-Hydroxy-4-Nitro-Anthraquinone; (5-hydroxyindolo[1,2-a]quinazolin-7-yl)acetic acid; dimethyl-(4,5,6,7-tetrabromo-1h-benzoimidazol-2-yl)-amine; N1,N2-ethylene-2-methylamino-4,5,6,7-tetrabromo-benzimidazole; 1,8-Di-Hydroxy-4-Nitro-Xanthen-9-One; 5,8-Di-Amino-1,4-Dihydroxy-Anthraquinone; 19-(cyclopropylamino)-4,6,7,15-tetrahydro-5H-16,1-(azenometheno)-10,14-(metheno)pyrazolo[4,3-o][1,3,9]triazacyclohexadecin-8(9H)-one; N,N′-diphenylpyrazolo[1,5-a][1,3,5]triazine-2,4-diamine; 4-(2-(1h-imidazol-4-yl)ethylamino)-2-(phenylamino)pyrazolo[1,5-a][1,3,5]triazine-8-carbonitrile; 2-(cyclohexylmethylamino)-4-(phenylamino)pyrazolo[1,5-a][1,3,5]triazine-8-carbonitrile; 2-(4-chlorobenzylamino)-4-(phenylamino)pyrazolo[1,5-a][1,3,5]triazine-8-carbonitrile; 2-(4-ethylpiperazin-1-yl)-4-(phenylamino)pyrazolo[1,5-a][1,3,5]triazine-8-carbonitrile; N-(3-(8-cyano-4-(phenylamino)pyrazolo[1,5-a][1,3,5]triazin-2-ylamino)phenyl)acetamide; Dichlororibofuranosylbenzimidazole; Quinalizarin; Ellagic acid; ATP; Quercetin; and Fostamatinib.

### Kinase Modulation—Further Embodiments

1. A method for selecting a treatment for a subject having or suspected of having Crohn's Disease, comprising:
   (a) obtaining a biological sample comprising gene expression products from the subject;
   (b) subjecting the biological sample to an assay to yield a data set including data corresponding to gene expression product levels;
   (c) in a programmed computer, inputting said data including said gene expression product levels from (b) to a trained algorithm to generate a classification of said sample as positive for a CD-PBmu subtype based on detection of an expression profile comprising an increase in the gene expression levels compared to a reference expression profile, wherein the trained algorithm is trained with a plurality of training samples, and wherein said biological sample is independent of said plurality of training samples;
   (d) electronically outputting a report that identifies the classification of the biological sample as positive for the CD-PBmu subtype; and
   (e) correlating the positive CD-PBmu subtype with a treatment comprising administration of a modulator of a kinase.
2. The method of embodiment 1, wherein the gene expression products comprises RNA.
3. The method of embodiment 1 or embodiment 2, wherein the assay comprises using one or more of a microarray, sequencing, and qPCR.
4. The method of any previous embodiment, wherein the trained algorithm is trained with one or more datasets of gene expression product levels obtained from the plurality of training samples.
5. The method of any previous embodiment, wherein the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof.
6. The method of embodiment 5, wherein the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.
7. The method of any previous embodiment, wherein the increase in the gene expression product levels is at least 2-fold greater than in the reference expression profile.
8. The method of any previous embodiment, wherein the reference expression profile comprises expression levels of the one or more genes of one or more subjects that do not have CD.
9. The method of any previous embodiment, wherein the biological sample comprises a blood sample or is purified from a blood sample of the subject.
10. The method of any previous embodiment, further comprising treating the subject by administering to the subject the kinase modulator.
11. The method of any previous embodiment, further comprising optimizing a therapeutic regimen of the subject comprising increasing or decreasing a dosage amount of the kinase modulator.
12. The method of any previous embodiment, wherein the kinase modulator comprises an inhibitor of a kinase.
13. The method of any previous embodiment, wherein the kinase modulator comprises one or more kinase modulators of Table 20B.
14. The method of any previous embodiment, wherein the kinase modulator comprises PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, or CSNK2A1, or a combination thereof.
15. The method of any previous embodiment, wherein expression of the kinase is elevated in the sample from the subject as compared to a reference expression profile of one or more subjects who do not comprise the CD PBmu subtype.
16. The method of any previous embodiment, comprising treating the subject with the kinase modulator.
17. A method of treating Crohn's disease (CD) in a subject, the method comprising administering to the subject a therapeutically effective amount of a kinase modulator, provided the subject is identified as having a CD-PBmu subtype by: (a) detecting an expression profile comprising an increase in a level of expression of one or more genes in a biological sample from the subject, relative to a reference expression profile; and (b) identifying the subject as having a CD-PBmu subtype based upon the expression profile that is detected in (b).
18. The method of embodiment 17, wherein the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPINGL UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A, or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.
19. The method of embodiment 18, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPINGL SOD3, SYK, TBC1D3, TBC1D9, TPSB2, MIR155HG, or UBD, or a combination thereof
20. The method of embodiment 18 or 19, wherein the one or more genes comprises at least 10 of the one or more genes.
21. The method of embodiment 18 or 19, wherein the one or more genes comprises between about 10-27 of the one or more genes.
22. The method of any one of embodiments 17-21, wherein the increase in the level of expression of the one or more genes in the biological sample is at least 2-fold greater than in the reference expression profile.
23. The method of any one of embodiments 17-22, wherein the reference expression profile comprises expression levels of the one or more genes of one or more subjects that do not have CD.
24. The method of any one of embodiments 17-23, wherein detecting the expression profile comprises detecting the increase in the level of expression of the one or more genes by:
   (a) contacting the biological sample with a nucleic acid primer and/or detectable nucleic acid probe; and
   (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.
25. The method of any one of embodiments 17-24, wherein the kinase modulator comprises an inhibitor of the kinase.
26. The method of any one of embodiments 17-25, wherein the kinase modulator comprises PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, or CSNK2A1, or a combination thereof
27. The method of any one of embodiments 17-26, wherein the kinase modulator comprises one or more kinase modulators of Table 20B.
28. The method of any one of embodiments 17-27, wherein expression of the kinase is elevated in the sample from the subject as compared to a reference expression profile of one or more subjects who do not comprise the CD PBmu subtype.
29. The method of any one of embodiments 17-28, comprising treating the subject with the kinase modulator.
30. A method of selecting a treatment for a subject having Crohn's Disease (CD), the method comprising:
   (a) measuring a level of expression of one or more genes from Tables 1A-1B in a biological sample obtained from the subject having CD;
   (b) detecting an expression profile comprising an increase in the level of expression of the one or more genes in the biological sample, relative to a reference expression profile; and
   (c) identifying the subject as a candidate for treatment with a modulator of a kinase based upon the expression profile that is detected in (b).
31. The method of embodiment 30, provided that the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPINGL UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.
32. The method of embodiment 31, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof
33. The method of embodiment 31 or 32, wherein the one or more genes comprises at least 10 of the one or more genes.
34. The method of any one of embodiments 30-33, wherein the increase in the level of expression of the one or more genes in the biological sample is at least 2-fold greater than in the reference expression profile.
35. The method of any one of embodiments 30-34, wherein the reference expression profile comprises expression levels of the one or more genes of one or more subjects that do not have CD.
36. The method of any one of embodiments 30-35, wherein measuring a level of expression of one or more genes comprises utilizing an assay selected from the group consisting of an RNA sequencing method, a microarray method, and quantitative polymerase chain reaction (qPCR).
37. The method of any one of embodiments 30-36, wherein measuring a level of expression of one or more genes comprises:
   (a) contacting the biological sample with a nucleic acid primer and/or detectable nucleic acid probe; and
   (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.
38. The method of any one of embodiments 30-37, further comprising treating the subject by administering the modulator of kinase to the subject.
39. The method of any one of embodiments 30-38, wherein the kinase modulator comprises an inhibitor of the kinase.
40. The method of any one of embodiments 30-39, wherein the kinase modulator comprises PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, or CSNK2A1, or a combination thereof.
41. The method of any one of embodiments 30-40, wherein the kinase modulator comprises one or more kinase modulators of Table 20B.
42. The method of any one of embodiments 30-41, further comprising optimizing a therapeutic regimen of the subject comprising increasing or decreasing a dosage amount of the modulator of the kinase administered to the subject for the treatment of the CD, based on the expression profile.
43. The method of any one of embodiments 30-42, provided the biological sample comprises a blood sample or is purified from a blood sample of the subject.
44. A method of treating an inflammatory disease in a subject, the method comprising: administering to the subject a modulator of a kinase, provided that a sample comprising gene expression products from the subject comprises a PBmu subtype based on detection of an expression profile comprising an increase in gene expression level of one or more gene products compared to a reference expression profile of the one or more gene products.
45. The method of embodiment 44, wherein the inflammatory disease comprises inflammatory bowel disease.
46. The method of embodiment 45, wherein the inflammatory bowel disease comprises Crohn's disease.
47. The method of any one of embodiments 44-46, wherein the gene products comprise RNA.
48. The method of any one of embodiments 44-47, wherein the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof.
49. The method of embodiment 48, wherein the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.
50. The method of any one of embodiments 44-49, wherein the increase in the gene expression product levels is at least 2-fold greater than in the reference expression profile.
51. The method of any one of embodiments 44-50, wherein the reference expression profile comprises expression levels of the one or more genes of one or more subjects that do not have CD.
52. The method of any one of embodiments 44-51, wherein the biological sample comprises a blood sample or is purified from a blood sample of the subject.
53. The method of any one of embodiments 44-52, further comprising optimizing a therapeutic regimen of the subject comprising increasing or decreasing a dosage amount of the kinase modulator.
54. The method of any one of embodiments 44-53, wherein the kinase modulator comprises an inhibitor of the kinase.
55. The method of any one of embodiments 44-54, wherein the kinase modulator comprises PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, or CSNK2A1, or a combination thereof.
56. The method of any one of embodiments 44-55, wherein the kinase modulator comprises kinase modulators of Table 20B.
57. The method of any previous embodiment, wherein the CD is associated with perianal disease/fistula.
58. The method of any previous embodiments, wherein the CD is associated with stricturing disease.
59. The method of any previous embodiments, wherein the CD is associated with recurrence.
60. The method of any previous embodiment, wherein the CD is associated with increased immune reactivity to a microbial antigen (e.g., ASCA).

Pharmaceutical Compositions, Formulations, and Methods of Administration

In one aspect, methods of treating a subject, e.g., a subject having a CD-PBmu subtype, monocyte 2 subtype, monocyte 1 subtype, or any combination thereof, involve administration of a pharmaceutical composition comprising a therapeutic agent described herein, e.g., a modulatory of expression and/or activity of a biomarker in Tables 1A-1B, Table 13, Table 16, or Table 17B, or of a biomolecule in a pathway of a biomarker in Table 14, or a modulator of miR-155, a therapeutic agent of Tables 3-13, or a combination thereof, in therapeutically effective amounts to said subject. In some embodiments, the subject has perianal disease/fistula, stricturing disease, recurrence, or increased immune reactivity to a microbial antigen, or a combination thereof. In some embodiments, the therapeutic agent comprises a modulator of a kinase, such as a kinase of Table 20A. In some embodiments, the kinase modulator comprises an agent of Table 20B. In some embodiments, a therapeutic agent described herein is used in the preparation of medicaments for treating an inflammatory disease, such as Crohn's Disease.

In certain embodiments, the compositions containing the therapeutic agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a therapeutic agent is administered to a patient suffering from an inflammatory disease such as CD, and optionally comprises a CD-PBmu subtype and/or monocyte 1 or 2 subtype.

In prophylactic applications, compositions containing a therapeutic agent described herein are administered to a patient susceptible to or otherwise at risk of a particular disease, disorder or condition, e.g., an inflammatory disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given therapeutic agent that corresponds to such an amount varies depending upon factors such as the particular therapeutic agent, disease condition and its severity, the identity (e.g., weight, sex, age) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a therapeutic agent, the patient is also weaned off (e.g., step-wise decrease in dose) a second treatment regimen.

In one embodiment, the daily dosages appropriate for a therapeutic agent herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED₅₀ with minimal toxicity. In certain embodiments, the daily dosage range and/or the unit dosage amount varies within this range depending upon the dosage form employed and the route of administration utilized.

Disclosed herein are therapeutic agents formulated into pharmaceutical compositions. Pharmaceutical compositions are formulated in a conventional manner using one or more pharmaceutically acceptable inactive ingredients that facilitate processing of the active therapeutic agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a therapeutic agent described herein, and at least one pharmaceutically acceptable inactive ingredient. In some embodiments, the therapeutic agents described herein are administered as pharmaceutical compositions in which the therapeutic agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH >7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

The disclosure also provides kits for detecting expression of one or more genes in Tables 1A-1B, Table 16, or Table 17A. Exemplary kits include nucleic acids configured for specific hybridization to one or more genes in Tables 1A-1B, Table 16, or Table 71A. In some cases a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, a gene in Tables 1A-1B, Table 16, or Table 17A. In some embodiments, the kit includes primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to a biomarker of Tables 1A-1B, Table 16, or Table 17A.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for detecting a particular subtype of IBD or CD in a subject. In some embodiments, the subtype is CD-PBmu. In some embodiments, the subtype is CD PBT. In some embodiments, the subtype is monocyte 2 subtype. In some embodiments, the subtype is monocyte 1 subtype. The system is configured to implement the methods described in this disclosure, including, but not limited to, detecting the presence of a particular CD subtype to determine whether the subject is suitable for treatment with a particular therapy.

In some embodiments, disclosed herein is a system for detecting a IBD subtype in a subject, comprising: (a) a computer processing device, optionally connected to a computer network; and (b) a software module executed by the computer processing device to analyze a target nucleic acid sequence of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof

Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C #, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

Web Browser Plus-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

Business Methods Utilizing a Computer

The gene expression profiling methods may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a gene expression profile may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Further Embodiments

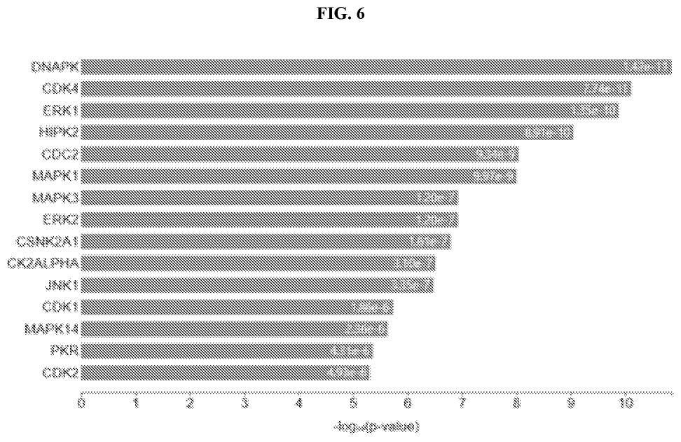

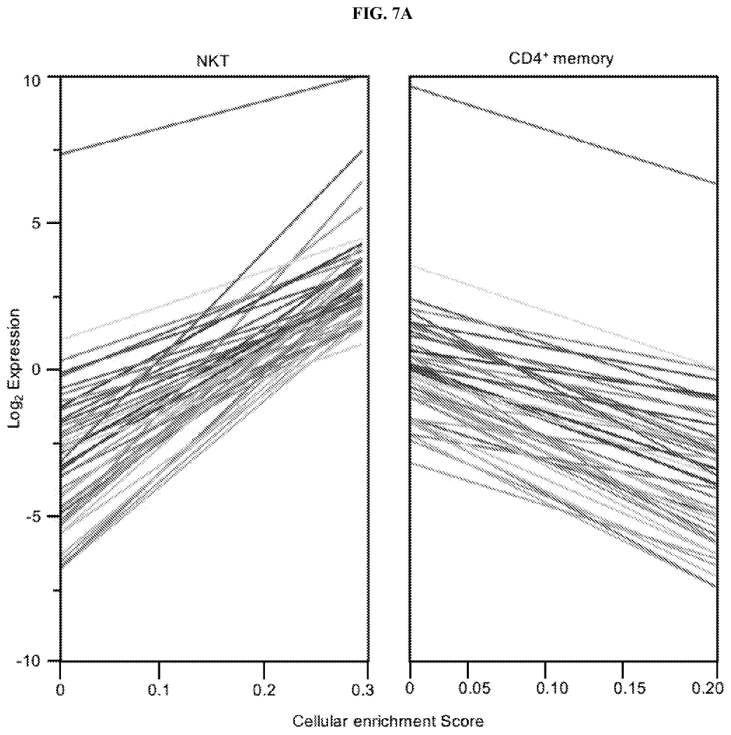

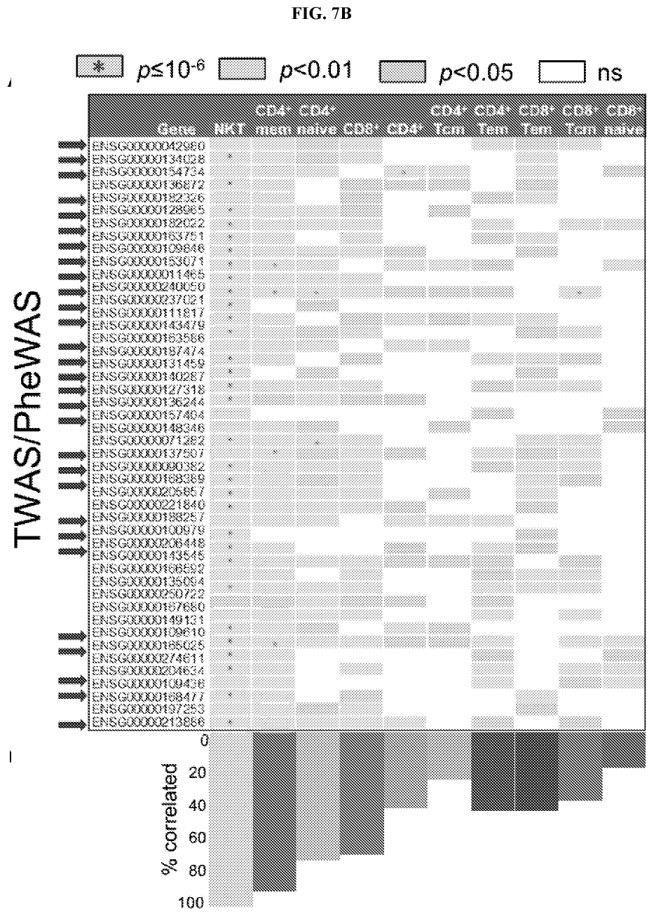

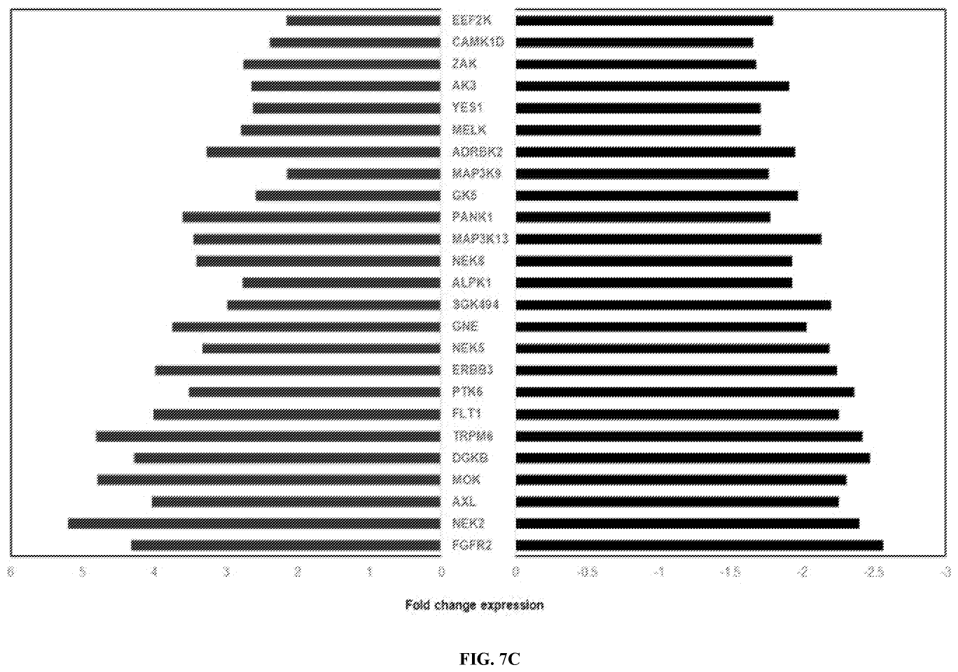

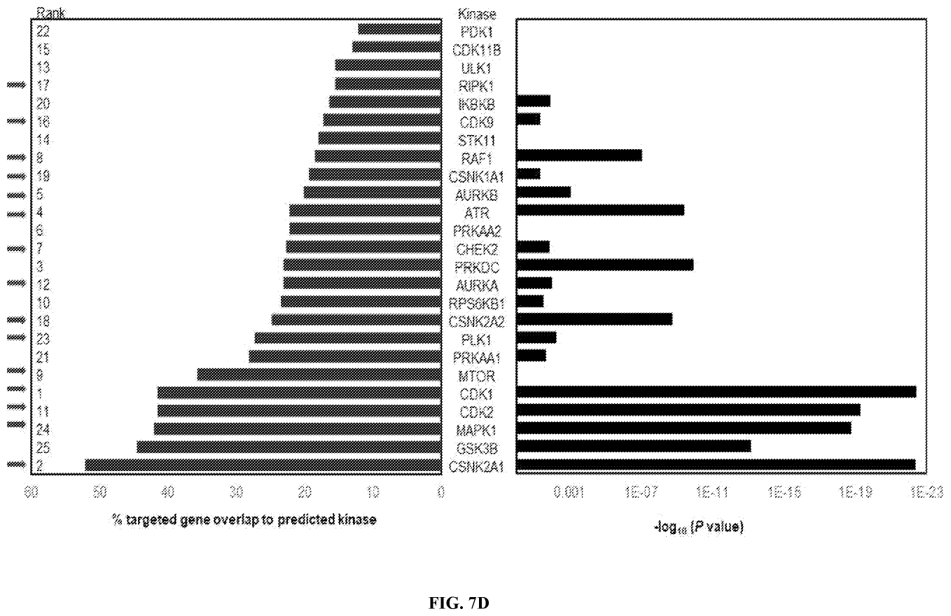

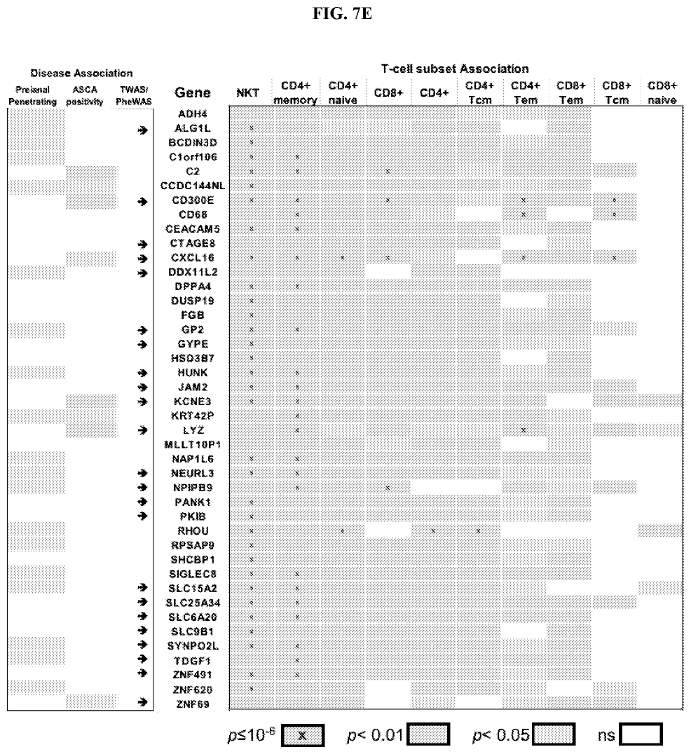

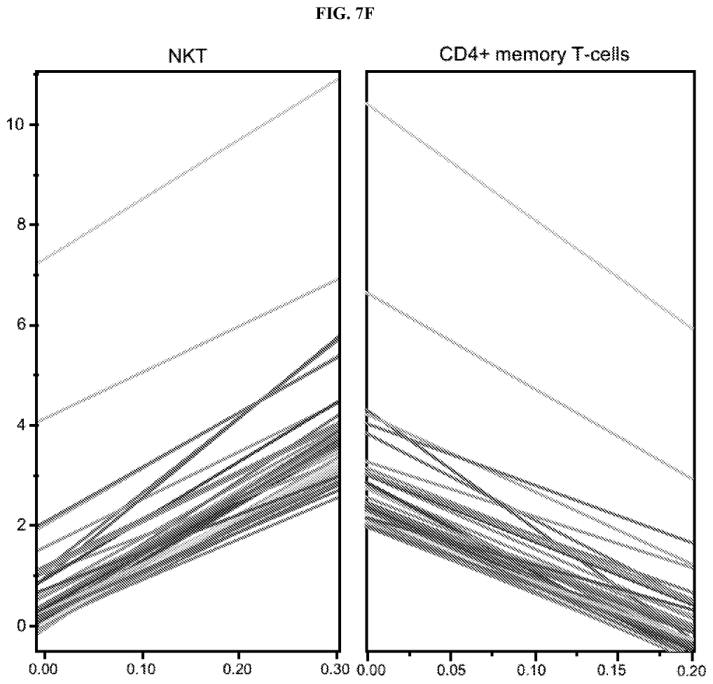

(1) A method for selecting a treatment for a subject having or suspected of having Crohn's Disease, comprising: (a) obtaining a biological sample comprising gene expression products from the subject; (b) subjecting the biological sample to an assay to yield a data set including data corresponding to gene expression product levels; (c) in a programmed computer, inputting said data including said gene expression product levels from (b) to a trained algorithm to generate a classification of said sample as positive for a CD-PBmu subtype based on detection of an expression profile comprising an increase in the gene expression levels compared to a reference expression profile, wherein the trained algorithm is trained with a plurality of training samples, and wherein said biological sample is independent of said plurality of training samples; (d) electronically outputting a report that identifies the classification of the biological sample as positive for the CD-PBmu subtype; and (e) correlating the positive CD-PBmu subtype with a treatment. (2) The method of embodiment 1, wherein the treatment comprises administration of a therapeutic agent comprising a therapeutic of Table 20B. (3) The method of embodiment 1, wherein the treatment comprises administration of a therapeutic agent that targets or modulates a molecule of Table 14. (4) The method of embodiment 1, wherein the treatment comprises administration of a therapeutic agent that targets or modulates a molecule of Table 15. (5) The method of embodiment 1, wherein the treatment comprises administration of a therapeutic agent that targets or modulates a molecule of Table 17A. (6) The method of embodiment 1, wherein the treatment comprises administration of a therapeutic agent that targets or modulates a molecule of Table 17B. (7) The method of embodiment 1, wherein the treatment comprises administration of a therapeutic agent that targets or modulates a molecule of Table 20A. (8) The method of embodiment 1, wherein the treatment comprises administration of a therapeutic agent that targets or modulates a molecule in a pathway of one or more genes of Table 17B. (9) The method of embodiment 1, wherein the treatment comprises administration of a therapeutic agent that modulates expression and/or activity of a molecule in a pathway of one or more genes of Table 1A. (10) The method of embodiment 1, wherein the treatment comprises administration of a therapeutic agent that modulates expression and/or activity of a molecule in a pathway of one or more genes of Table 1B. (11) The method of embodiment 1, wherein the treatment comprises administration of a therapeutic agent that targets a molecule in a pathway of one or more genes of Table 1A. (12) The method of embodiment 1, wherein the treatment comprises administration of a therapeutic agent that targets a molecule in a pathway of one or more genes of Table 1B. (13) The method of embodiment 1, wherein the treatment comprises administration of a therapeutic agent comprising a kinase inhibitor. (14) The method of embodiment 13, wherein the kinase target of the kinase inhibitor is a kinase described herein. (15) The method of embodiment 13, wherein the kinase target of the kinase inhibitor comprises a kinase of FIG. 6. (16) The method of embodiment 13, wherein the kinase target of the kinase inhibitor comprises a kinase of FIG. 7C. (17) The method of embodiment 13, wherein the kinase target of the kinase inhibitor comprises a kinase of FIG. 7D. (18) The method of embodiment 1, wherein the treatment comprises administration of a therapeutic agent comprising an anti-TL1A antibody. (19) The method of embodiment 1, wherein the treatment comprises administration of a therapeutic agent comprising a modulator of miR-155. (20) The method of embodiment 19, comprising treating the subject with the miR-155 modulator. (21) The method of embodiment 19 or embodiment 20, further comprising optimizing a therapeutic regimen of the subject comprising increasing or decreasing a dosage amount of the miR-155 modulator. (22) The method of any one of embodiments 19-21, wherein the miR-155 modulator comprises an inhibitor of miR-155. (23) The method of any one of embodiments 19-22, wherein the miR-155 modulator comprises one or more oligonucleotides of Tables 3-12. (24) The method of any one of embodiments 19-22, wherein the miR-155 modulator comprises Cobomarsen. (25) The method of any one of embodiments 19-24, wherein expression of miR-155 is elevated in the sample from the subject as compared to a reference expression profile of one or more subjects who do not comprise the CD PBmu subtype.

(26) The method of any previous embodiment, wherein the gene expression products comprise RNA. (27) The method of any previous embodiment, wherein the assay comprises using one or more of a microarray, sequencing, and qPCR. (28) The method of any previous embodiment, wherein the trained algorithm is trained with one or more datasets of gene expression product levels obtained from the plurality of training samples. (29) The method of any previous embodiment, wherein the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof (30) The method of any previous embodiment, wherein the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof (31) The method of any previous embodiment, wherein the increase in the gene expression product levels is at least 2-fold greater than in the reference expression profile. (32) The method of any previous embodiment, wherein the reference expression profile comprises expression levels of the one or more genes of one or more subjects that do not have CD. (33) The method of any previous embodiment, wherein the biological sample comprises a blood sample or is purified from a blood sample of the subject.

(34) A method for selecting a treatment for a subject having or suspected of having Crohn's Disease, comprising: (a) obtaining a biological sample comprising MIR155 from the subject; (b) subjecting the biological sample to an assay to yield a data set including data corresponding to expression level of the MIR155; (c) in a programmed computer, inputting said data including said expression level of the MIR155 from (b) to a trained algorithm to generate a classification of said sample as positive for a subtype based on detection of an expression profile comprising an increase in the expression level of MIR155 compared to a reference expression profile, wherein the trained algorithm is trained with a plurality of training samples, and wherein said biological sample is independent of said plurality of training samples; (d) electronically outputting a report that identifies the classification of the biological sample as positive for the subtype; and (e) correlating the positive subtype with a treatment. (35) The method of embodiment 34, wherein the treatment comprises administration of a therapeutic agent comprising a therapeutic of Table 20B. (36) The method of embodiment 34, wherein the treatment comprises administration of a therapeutic agent that targets or modulates a molecule of Table 14. (37) The method of embodiment 34, wherein the treatment comprises administration of a therapeutic agent that targets or modulates a molecule of Table 15. (38) The method of embodiment 34, wherein the treatment comprises administration of a therapeutic agent that targets or modulates a molecule of Table 17A. (39) The method of embodiment 34, wherein the treatment comprises administration of a therapeutic agent that targets or modulates a molecule of Table 17B. (40) The method of embodiment 34, wherein the treatment comprises administration of a therapeutic agent that targets or modulates a molecule of Table 20A. (41) The method of embodiment 34, wherein the treatment comprises administration of a therapeutic agent that targets or modulates a molecule in a pathway of one or more genes of Table 17B. (42) The method of embodiment 34, wherein the treatment comprises administration of a therapeutic agent that modulates expression and/or activity of a molecule in a pathway of one or more genes of Table 1A. (43) The method of embodiment 34, wherein the treatment comprises administration of a therapeutic agent that modulates expression and/or activity of a molecule in a pathway of one or more genes of Table 1B. (44) The method of embodiment 34, wherein the treatment comprises administration of a therapeutic agent that targets a molecule in a pathway of one or more genes of Table 1A. (45) The method of embodiment 34, wherein the treatment comprises administration of a therapeutic agent that targets a molecule in a pathway of one or more genes of Table 1B. (46) The method of embodiment 34, wherein the treatment comprises administration of a therapeutic agent comprising a kinase inhibitor. (47) The method of embodiment 46, wherein the kinase target of the kinase inhibitor is a kinase described herein. (48) The method of embodiment 46, wherein the kinase target of the kinase inhibitor comprises a kinase of FIG. 6. (49) The method of embodiment 46, wherein the kinase target of the kinase inhibitor comprises a kinase of FIG. 7C. (50) The method of embodiment 46, wherein the kinase target of the kinase inhibitor comprises a kinase of FIG. 7D. (51) The method of embodiment 34, wherein the treatment comprises administration of a therapeutic agent comprising an anti-TL1A antibody. (52) The method of embodiment 34, wherein the treatment comprises administration of a therapeutic agent comprising a modulator of miR-155. (53) The method of embodiment 52, comprising treating the subject with the miR-155 modulator. (54) The method of embodiment 52 or embodiment 53, further comprising optimizing a therapeutic regimen of the subject comprising increasing or decreasing a dosage amount of the miR-155 modulator. (55) The method of any one of embodiments 52-54, wherein the miR-155 modulator comprises an inhibitor of miR-155. (56) The method of any one of embodiments 52-55, wherein the miR-155 modulator comprises one or more oligonucleotides of Tables 3-12. (57) The method of any one of embodiments 52-56, wherein the miR-155 modulator comprises Cobomarsen.

(58) The method of any one of embodiments 34-57, wherein the assay comprises using one or more of a microarray, sequencing, and qPCR. (59) The method of any one of embodiments 34-58, wherein the increase in the gene expression product levels is at least 2-fold greater than in the reference expression profile. (60) The method of any one of embodiments 34-59, wherein the reference expression profile comprises expression levels of MIR155 of one or more subjects that do not have CD. (61) The method of any one of embodiments 34-60, wherein the biological sample comprises a blood sample or is purified from a blood sample of the subject. (62) The method of any one of embodiments 34-61, further comprising treating the subject by administering to the subject a miR-155 modulator. (63) The method of any one of embodiments 34-62, further comprising optimizing a therapeutic regimen of the subject comprising increasing or decreasing a dosage amount of a miR-155 modulator. (64) The method of embodiment 62 or embodiment 63, wherein the miR-155 modulator comprises an inhibitor of miR-155. (65) The method of any one of embodiments 62-64, wherein the miR-155 modulator comprises one or more oligonucleotides of Tables 3-12. (66) The method of any one of embodiments 62-64, wherein the miR-155 modulator comprises Cobomarsen.

(67) A method of treating Crohn's disease (CD) in a subject, the method comprising administering to the subject a therapeutically effective amount of a therapeutic agent, provided the subject is identified as having a CD-PBmu subtype by: (a) detecting an expression profile comprising an increase in a level of expression of one or more genes in a biological sample from the subject, relative to a reference expression profile; and (b) identifying the subject as having a CD-PBmu subtype based upon the expression profile that is detected in (b). (68) The method of embodiment 67, wherein the therapeutic agent comprises a therapeutic of Table 20B. (69) The method of embodiment 67, wherein the therapeutic agent targets or modulates a molecule of Table 14. (70) The method of embodiment 67, wherein the therapeutic agent targets or modulates a molecule of Table 15. (71) The method of embodiment 67, wherein the therapeutic agent targets or modulates a molecule of Table 17A. (72) The method of embodiment 67, wherein the therapeutic agent targets or modulates a molecule of Table 17B. (73) The method of embodiment 67, wherein the therapeutic agent targets or modulates a molecule of Table 20A. (74) The method of embodiment 67, wherein the therapeutic agent targets or modulates a molecule in a pathway of one or more genes of Table 17B. (75) The method of embodiment 67, wherein the therapeutic agent modulates expression and/or activity of a molecule in a pathway of one or more genes of Table 1A. (76) The method of embodiment 67, wherein the therapeutic agent modulates expression and/or activity of a molecule in a pathway of one or more genes of Table 1B. (77) The method of embodiment 67, wherein the therapeutic agent targets a molecule in a pathway of one or more genes of Table 1A. (78) The method of embodiment 67, wherein the therapeutic agent targets a molecule in a pathway of one or more genes of Table 1B. (79) The method of embodiment 67, wherein the therapeutic agent comprising a kinase inhibitor. (80) The method of embodiment 79, wherein the kinase target of the kinase inhibitor is a kinase described herein. (81) The method of embodiment 79, wherein the kinase target of the kinase inhibitor comprises a kinase of FIG. 6. (82) The method of embodiment 79, wherein the kinase target of the kinase inhibitor comprises a kinase of FIG. 7C. (83) The method of embodiment 79, wherein the kinase target of the kinase inhibitor comprises a kinase of FIG. 7D. (84) The method of embodiment 67, wherein the therapeutic agent comprises an anti-TL1A antibody. (85) The method of embodiment 67, wherein the therapeutic agent comprises a modulator of miR-155. (86) The method of embodiment 85, comprising treating the subject with the miR-155 modulator. (87) The method of embodiment 85 or embodiment 86, further comprising optimizing a therapeutic regimen of the subject comprising increasing or decreasing a dosage amount of the miR-155 modulator. (88) The method of any one of embodiments 85-87, wherein the miR-155 modulator comprises an inhibitor of miR-155. (89) The method of any one of embodiments 85-88, wherein the miR-155 modulator comprises one or more oligonucleotides of Tables 3-12. (90) The method of any one of embodiments 85-88, wherein the miR-155 modulator comprises Cobomarsen.

(91) The method of any one of embodiments 67-90, wherein the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A, or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof (92) The method of any one of embodiments 67-90, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof. (93) The method of any one of embodiments 67-92, wherein the one or more genes comprises at least 10 of the one or more genes. (94) The method of any one of embodiments 67-93, wherein the one or more genes comprises between about 10-27 of the one or more genes. (95) The method of any one of embodiments 67-94, wherein the increase in the level of expression of the one or more genes in the biological sample is at least 2-fold greater than in the reference expression profile. (96) The method of any one of embodiments 67-95, wherein the reference expression profile comprises expression levels of the one or more genes of one or more subjects that do not have CD. (97) The method of any one of embodiments 67-96, wherein detecting the expression profile comprises detecting the increase in the level of expression of the one or more genes by: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. (98) The method of any one of embodiments 67-97, wherein expression of miR-155 is elevated in the sample from the subject as compared to a reference expression profile of one or more subjects who do not comprise the CD PBmu subtype.

(99) A method of treating Crohn's disease (CD) in a subject, the method comprising administering to the subject a therapeutically effective amount of a therapeutic agent, provided the subject is identified as having a CD-PBmu subtype by: (a) detecting an expression profile comprising an increase in a level of expression of MIR155 in a biological sample from the subject, relative to a reference expression profile; and (b) identifying the subject as having a CD-PBmu subtype based upon the expression profile that is detected in (b). (100) The method of embodiment 99, wherein the therapeutic agent comprises a therapeutic of Table 20B. (101) The method of embodiment 99, wherein the therapeutic agent targets or modulates a molecule of Table 14. (102) The method of embodiment 99, wherein the therapeutic agent targets or modulates a molecule of Table 15. (103) The method of embodiment 99, wherein the therapeutic agent targets or modulates a molecule of Table 17A. (104) The method of embodiment 99, wherein the therapeutic agent targets or modulates a molecule of Table 17B. (105) The method of embodiment 99, wherein the therapeutic agent targets or modulates a molecule of Table 20A. (106) The method of embodiment 99, wherein the therapeutic agent targets or modulates a molecule in a pathway of one or more genes of Table 17B. (107) The method of embodiment 99, wherein the therapeutic agent modulates expression and/or activity of a molecule in a pathway of one or more genes of Table 1A. (108) The method of embodiment 99, wherein the therapeutic agent modulates expression and/or activity of a molecule in a pathway of one or more genes of Table 1B. (109) The method of embodiment 99, wherein the therapeutic agent targets a molecule in a pathway of one or more genes of Table 1A. (110) The method of embodiment 99, wherein the therapeutic agent targets a molecule in a pathway of one or more genes of Table 1B. (111) The method of embodiment 99, wherein the therapeutic agent comprises a kinase inhibitor. (112) The method of embodiment 111, wherein the kinase target of the kinase inhibitor is a kinase described herein. (113) The method of embodiment 111, wherein the kinase target of the kinase inhibitor comprises a kinase of FIG. 6. (114) The method of embodiment 111, wherein the kinase target of the kinase inhibitor comprises a kinase of FIG. 7C. (115) The method of embodiment 111, wherein the kinase target of the kinase inhibitor comprises a kinase of FIG. 7D. (116) The method of embodiment 99, wherein the therapeutic agent comprises an anti-TL1A antibody. (117) The method of embodiment 99, wherein the therapeutic agent comprises a modulator of miR-155. (118) The method of embodiment 117, comprising treating the subject with the miR-155 modulator. (119) The method of embodiment 117 or embodiment 118, further comprising optimizing a therapeutic regimen of the subject comprising increasing or decreasing a dosage amount of the miR-155 modulator. (120) The method of any one of embodiments 117-119, wherein the miR-155 modulator comprises an inhibitor of miR-155. (121) The method of any one of embodiments 117-119, wherein the miR-155 modulator comprises one or more oligonucleotides of Tables 3-12. (122) The method of any one of embodiments 117-119, wherein the miR-155 modulator comprises Cobomarsen.

(123) The method of any one of embodiments 99-122, wherein the increase in the level of expression of MIR155 in the biological sample is at least 2-fold greater than in the reference expression profile. (124) The method of any one of embodiments 99-123, wherein the reference expression profile comprises expression levels of MIR155 of one or more subjects that do not have CD. (125) The method of any one of embodiments 99-124, wherein detecting the expression profile comprises detecting the increase in the level of expression of MIR155 by:
(a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. (126) The method of any one of embodiments 99-125, wherein the miR-155 modulator comprises an inhibitor of miR-155. (127) The method of any one of embodiments 99-126, wherein the miR-155 modulator comprises one or more oligonucleotides of Tables 3-12. (128) The method of any one of embodiments 99-127, wherein the miR-155 modulator comprises Cobomarsen. (129) The method of any one of embodiments 99-127, comprising treating the subject with the miR-155 modulator.

(130) A method of selecting a treatment for a subject having Crohn's Disease (CD), the method comprising: (a) measuring a level of expression of one or more genes from Tables 1A-1B in a biological sample obtained from the subject having CD; (b) detecting an expression profile comprising an increase in the level of expression of the one or more genes in the biological sample, relative to a reference expression profile; and (c) identifying the subject as a candidate for treatment based upon the expression profile that is detected in (b). (131) The method of embodiment 130, wherein the treatment comprises administration of a therapeutic agent comprising a therapeutic of Table 20B. (132) The method of embodiment 130, wherein the treatment comprises administration of a therapeutic agent that targets or modulates a molecule of Table 14. (133) The method of embodiment 130, wherein the treatment comprises administration of a therapeutic agent that targets or modulates a molecule of Table 15. (134) The method of embodiment 130, wherein the treatment comprises administration of a therapeutic agent that targets or modulates a molecule of Table 17A. (135) The method of embodiment 130, wherein the treatment comprises administration of a therapeutic agent that targets or modulates a molecule of Table 17B. (136) The method of embodiment 130, wherein the treatment comprises administration of a therapeutic agent that targets or modulates a molecule of Table 20A. (137) The method of embodiment 130, wherein the treatment comprises administration of a therapeutic agent that targets or modulates a molecule in a pathway of one or more genes of Table 17B. (138) The method of embodiment 130, wherein the treatment comprises administration of a therapeutic agent that modulates expression and/or activity of a molecule in a pathway of one or more genes of Table 1A. (139) The method of embodiment 130, wherein the treatment comprises administration of a therapeutic agent that modulates expression and/or activity of a molecule in a pathway of one or more genes of Table 1B. (140) The method of embodiment 130, wherein the treatment comprises administration of a therapeutic agent that targets a molecule in a pathway of one or more genes of Table 1A. (141) The method of embodiment 130, wherein the treatment comprises administration of a therapeutic agent that targets a molecule in a pathway of one or more genes of Table 1B. (142) The method of embodiment 130, wherein the treatment comprises administration of a therapeutic agent comprising a kinase inhibitor. (143) The method of embodiment 142, wherein the kinase target of the kinase inhibitor is a kinase described herein. (144) The method of embodiment 142, wherein the kinase target of the kinase inhibitor comprises a kinase of FIG. 6. (145) The method of embodiment 142, wherein the kinase target of the kinase inhibitor comprises a kinase of FIG. 7C. (146) The method of embodiment 142, wherein the kinase target of the kinase inhibitor comprises a kinase of FIG. 7D. (147) The method of embodiment 130, wherein the treatment comprises administration of a therapeutic agent comprising an anti-TL1A antibody. (148) The method of embodiment 130, wherein the treatment comprises administration of a therapeutic agent comprising a modulator of miR-155. (149) The method of embodiment 148, comprising treating the subject with the miR-155 modulator. (150) The method of embodiment 148 or embodiment 149, further comprising optimizing a therapeutic regimen of the subject comprising increasing or decreasing a dosage amount of the miR-155 modulator. (151) The method of any one of embodiments 148-150, wherein the miR-155 modulator comprises an inhibitor of miR-155. (152) The method of any one of embodiments 148-151, wherein the miR-155 modulator comprises one or more oligonucleotides of Tables 3-12. (153) The method of any one of embodiments 148-152, wherein the miR-155 modulator comprises Cobomarsen. (154) The method of any one of embodiments 148-153, wherein expression of miR-155 is elevated in the sample from the subject as compared to a reference expression profile of one or more subjects who do not comprise the CD PBmu subtype.

(155) The method of any one of embodiments 130-154, wherein the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof (156) The method of any one of embodiments 130-155, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof. (157) The method of any one of embodiments 130-156, wherein the one or more genes comprises at least 10 of the one or more genes. (158) The method of any one of embodiments 130-157, wherein the increase in the level of expression of the one or more genes in the biological sample is at least 2-fold greater than in the reference expression profile. (159) The method of any one of embodiments 130-158, wherein the reference expression profile comprises expression levels of the one or more genes of one or more subjects that do not have CD. (160) The method of any one of embodiments 130-159, wherein measuring a level of expression of one or more genes comprises utilizing an assay selected from the group consisting of an RNA sequencing method, a microarray method, and quantitative polymerase chain reaction (qPCR). (161) The method of any one of embodiments 130-160, wherein measuring a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. (162) The method of any one of embodiments 130-161, further comprising treating the subject by administering a modulator of miR-155 to the subject. (163) The method of embodiment 162, wherein the miR-155 modulator comprises an inhibitor of miR-155. (164) The method of embodiment 162, wherein the miR-155 modulator comprises one or more oligonucleotides of Tables 3-12. (165) The method of embodiment 162, wherein the miR-155 modulator comprises Cobomarsen. (166) The method of any one of embodiments 162-165, further comprising optimizing a therapeutic regimen of the subject comprising increasing or decreasing a dosage amount of the modulator of miR-155 administered to the subject for the treatment of the CD, based on the expression profile. (167) The method of any one of embodiments 130-166, provided the biological sample comprises a blood sample or is purified from a blood sample of the subject.

(168) A method of determining a Crohn's Disease (CD) subtype in a subject having CD, the method comprising: (a) measuring a level of expression of MIR155 in a biological sample obtained from a subject having CD; (b) detecting an expression profile comprising an increase in the level of expression of MIR155 in the biological sample, relative to a reference expression profile; and (c) identifying the subject as having a CD-PBmu subtype based upon the expression profile that is detected in (b). (169) The method of embodiment 168, wherein the increase in the level of expression of MIR155 in the biological sample is at least 2-fold greater than in the reference expression profile. (170) The method of embodiment 168 or embodiment 169, wherein the reference expression profile comprises expression levels of MIR155 of one or more subjects that do not have CD. (171) The method of any one of embodiments 168-170, wherein measuring a level of expression comprises utilizing an assay selected from the group consisting of an RNA sequencing method, a microarray method, and quantitative polymerase chain reaction (qPCR). (172) The method of any one of embodiments 168-171, wherein measuring a level of expression of MIR155 comprises: (a) contacting the biological sample with a nucleic acid primer and/or detectable nucleic acid probe; and (b) hybridizing the nucleic acid primer and/or detectable nucleic acid probe to a nucleic acid sequence of MIR155, wherein the detectable nucleic acid probe comprises a nucleic acid sequence comprising at least about 10 contiguous nucleic acids of MIR155. (173) The method of any one of embodiments 168-172, further comprising treating the subject by administering a therapeutic agent to the subject. (174) The method of embodiment 173, wherein the therapeutic agent comprises a therapeutic of Table 20B. (175) The method of embodiment 173, wherein the therapeutic agent targets or modulates a molecule of Table 14. (176) The method of embodiment 173, wherein the therapeutic agent targets or modulates a molecule of Table 15. (177) The method of embodiment 173, wherein the therapeutic agent targets or modulates a molecule of Table 17A. (178) The method of embodiment 173, wherein the therapeutic agent targets or modulates a molecule of Table 17B. (179) The method of embodiment 173, wherein the therapeutic agent targets or modulates a molecule of Table 20A. (180) The method of embodiment 173, wherein the therapeutic agent targets or modulates a molecule in a pathway of one or more genes of Table 17B. (181) The method of embodiment 173, wherein the therapeutic agent modulates expression and/or activity of a molecule in a pathway of one or more genes of Table 1A. (182) The method of embodiment 173, wherein the therapeutic agent modulates expression and/or activity of a molecule in a pathway of one or more genes of Table 1B. (183) The method of embodiment 173, wherein the therapeutic agent targets a molecule in a pathway of one or more genes of Table 1A. (184) The method of embodiment 173, wherein the therapeutic agent targets a molecule in a pathway of one or more genes of Table 1B. (185) The method of embodiment 173, wherein the therapeutic agent comprises a kinase inhibitor. (186) The method of embodiment 185, wherein the kinase target of the kinase inhibitor is a kinase described herein. (187) The method of embodiment 185, wherein the kinase target of the kinase inhibitor comprises a kinase of FIG. 6. (188) The method of embodiment 185, wherein the kinase target of the kinase inhibitor comprises a kinase of FIG. 7C. (189) The method of embodiment 185, wherein the kinase target of the kinase inhibitor comprises a kinase of FIG. 7D. (190) The method of embodiment 173, wherein the therapeutic agent comprises an anti-TL1A antibody. (191) The method of embodiment 173, wherein the therapeutic agent comprises a modulator of miR-155. (192) The method of embodiment 191, comprising treating the subject with the miR-155 modulator. (193) The method of embodiment 191 or embodiment 191, further comprising optimizing a therapeutic regimen of the subject comprising increasing or decreasing a dosage amount of the miR-155 modulator. (194) The method of any one of embodiments 191-193, wherein the miR-155 modulator comprises an inhibitor of miR-155. (195) The method of any one of embodiments 191-194, wherein the miR-155 modulator comprises one or more oligonucleotides of Tables 3-12. (196) The method of any one of embodiments 191-195, wherein the miR-155 modulator comprises Cobomarsen.

(197) The method of any one of embodiments 168-196, further comprising optimizing a therapeutic regimen of the subject comprising increasing or decreasing a dosage amount of the therapeutic agent administered to the subject for the treatment of the CD, based on the CD-PBmu subtype. (198) The method of embodiment 197, wherein the therapeutic agent comprises a miR-155 modulator. (199) The method of embodiment 198, wherein the miR-155 modulator comprises an inhibitor of miR-155. (200) The method of embodiment 198 or embodiment 199, wherein the miR-155 modulator comprises one or more oligonucleotides of Tables 3-12. (201) The method of embodiment 198 or embodiment 199, wherein the miR-155 modulator comprises Cobomarsen. (202) The method of any one of embodiments 168-201, provided the biological sample comprises a blood sample or is purified from a blood sample of the subject.

(203) A method of treating an inflammatory disease in a subject, the method comprising: administering to the subject a therapeutic agent, wherein a sample comprising gene expression products from the subject comprises a PBmu subtype based on detection of an expression profile comprising an increase in gene expression level of one or more gene products compared to a reference expression profile of the one or more gene products. (204) The method of embodiment 203, wherein the therapeutic agent comprises a therapeutic of Table 20B. (205) The method of embodiment 203, wherein the therapeutic agent targets or modulates a molecule of Table 14. (206) The method of embodiment 203, wherein the therapeutic agent targets or modulates a molecule of Table 15. (207) The method of embodiment 203, wherein the therapeutic agent targets or modulates a molecule of Table 17A. (208) The method of embodiment 203, wherein the therapeutic agent targets or modulates a molecule of Table 17B. (209) The method of embodiment 203, wherein the therapeutic agent targets or modulates a molecule of Table 20A. (210) The method of embodiment 203, wherein the therapeutic agent targets or modulates a molecule in a pathway of one or more genes of Table 17B. (211) The method of embodiment 203, wherein the therapeutic agent modulates expression and/or activity of a molecule in a pathway of one or more genes of Table 1A. (212) The method of embodiment 203, wherein the therapeutic agent modulates expression and/or activity of a molecule in a pathway of one or more genes of Table 1B. (213) The method of embodiment 203, wherein the therapeutic agent targets a molecule in a pathway of one or more genes of Table 1A. (214) The method of embodiment 203, wherein the therapeutic agent targets a molecule in a pathway of one or more genes of Table 1B. (215) The method of embodiment 203, wherein the therapeutic agent comprises a kinase inhibitor. (216) The method of embodiment 215, wherein the kinase target of the kinase inhibitor is a kinase described herein. (217) The method of embodiment 215, wherein the kinase target of the kinase inhibitor comprises a kinase of FIG. 6. (218) The method of embodiment 215, wherein the kinase target of the kinase inhibitor comprises a kinase of FIG. 7C. (219) The method of embodiment 215, wherein the kinase target of the kinase inhibitor comprises a kinase of FIG. 7D. (220) The method of embodiment 203, wherein the therapeutic agent comprises an anti-TL1A antibody. (221) The method of embodiment 203, wherein the therapeutic agent comprises a modulator of miR-155. (222) The method of embodiment 221, comprising treating the subject with the miR-155 modulator. (223) The method of embodiment 221 or embodiment 222, further comprising optimizing a therapeutic regimen of the subject comprising increasing or decreasing a dosage amount of the miR-155 modulator. (224) The method of any one of embodiments 221-223, wherein the miR-155 modulator comprises an inhibitor of miR-155. (225) The method of any one of embodiments 221-224, wherein the miR-155 modulator comprises one or more oligonucleotides of Tables 3-12. (226) The method of any one of embodiments 221-225, wherein the miR-155 modulator comprises Cobomarsen.

(227) The method of any one of embodiments 203-226, wherein the inflammatory disease comprises inflammatory bowel disease. (228) The method of embodiment 227, wherein the inflammatory bowel disease comprises Crohn's disease. (229) The method of any one of embodiments 203-228, wherein the gene products comprise RNA. (230) The method of any one of embodiments 203-229, wherein the gene expression products are expressed from genes comprising one, two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), MIR155HG, phospholipase A2 group IIA (PLA2G2A), alcohol dehydrogenase 4 (class II) pi polypeptide (ADH4), ALG1 chitobiosyldiphosphodolichol beta-mannosyltransferase-like (ALG1L), BCDIN3 domain containing (BCDIN3D), chromosome 1 open reading frame 106 (C1orf106), complement component 2 (C2), coiled-coil domain containing 144 family N-terminal like (CCDC144NL), carcinoembryonic antigen-related cell adhesion molecule 5 (CEACAM5), CTAGE family member 8 (CTAGE8), DEAD/H (Asp-Glu-Ala-Asp/His) box helicase 11 like 2 (DDX11L2), developmental pluripotency associated 4 (DPPA4), dual specificity phosphatase 19 (DUSP19), fibrinogen beta chain (FGB), glycoprotein 2 (zymogen granule membrane) (GP2), glycophorin E (MNS blood group) (GYPE), hydroxy-delta-5-steroid dehydrogenase, 3 beta- and steroid delta-isomerase 7 (HSD3B7), hormonally up-regulated Neu-associated kinase (HUNK), junctional adhesion molecule 2 (JAM2), potassium channel voltage gated subfamily E regulatory beta subunit 3 (KCNE3), keratin 42 pseudogene (KRT42P), lysozyme (LYZ), myeloid/lymphoid or mixed-lineage leukemia translocated to 10 pseudogene 1 (MLLT10P1), nucleosome assembly protein 1-like 6 (NAP1L6), neuralized E3 ubiquitin protein ligase 3 (NEURL3), nuclear pore complex interacting protein family member B9 (NPIPB9), pantothenate kinase 1 (PANK1), protein kinase (cAMP-dependent, catalytic) inhibitor beta (PKIB), ras homolog family member U (RHOU), ribosomal protein SA pseudogene 9 (RPSAP9), SHC SH2-domain binding protein 1 (SHCBP1), sialic acid binding Ig-like lectin 8 (SIGLEC8), solute carrier family 15 (oligopeptide transporter) member 2 (SLC15A2), solute carrier family 25 member 34 (SLC25A34), solute carrier family 6 (proline IMINO transporter) member 20 (SLC6A20), solute carrier family 9 subfamily B (NHA1, cation proton antiporter 1) member 1 (SLC9B1), synaptopodin 2-like (SYNPO2L), teratocarcinoma-derived growth factor 1 (TDGF1), zinc finger protein 491 (ZNF491), zinc finger protein 620 (ZNF620), zinc finger protein 69 (ZNF69), chemokine (C-X-C motif) ligand 16 (CXCL16), CD68 molecule (CD68), or CD300e molecule (CD300E), or a combination thereof (231) The method of any one of embodiments 203-230, wherein the gene expression products are expressed from genes comprising (a) one, two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof, and/or (b) one, two or more of ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof (232) The method of any one of embodiments 203-231, wherein the increase in the gene expression product levels is at least 2-fold greater than in the reference expression profile. (233) The method of any one of embodiments 203-232, wherein the reference expression profile comprises expression levels of the one or more genes of one or more subjects that do not have CD. (234) The method of any one of embodiments 203-233, wherein the biological sample comprises a blood sample or is purified from a blood sample of the subject. (235) The method of any one of embodiments 203-234, further comprising optimizing a therapeutic regimen of the subject comprising increasing or decreasing a dosage amount of an miR-155 modulator. (236) The method of embodiment 235, wherein the miR-155 modulator comprises an inhibitor of miR-155. (237) The method of embodiment 235, wherein the miR-155 modulator comprises one or more oligonucleotides of Tables 3-12. (238) The method of embodiment 235, wherein the miR-155 modulator comprises Cobomarsen.

(239) The method of any previous embodiment, wherein the CD is associated with perianal disease/fistula. (240) The method of any previous embodiment, wherein the CD is associated with stricturing disease. (241) The method of any previous embodiment, wherein the CD is associated with recurrence. (242) The method of any previous embodiment, wherein the CD is associated with increased immune reactivity to a microbial antigen (e.g., ASCA).

(243) A method of determining a Crohn's Disease (CD) subtype status in a subject having CD, wherein the status comprises distinguishing a CD PBmucosal (CD-PBmu) subtype from a non-CD-PBmu subtype, the method comprising: detecting expression of one or more genes from Tables 1A-1B in a biological sample from the subject to obtain an expression profile comprising the expression levels of each of the one or more genes in the biological sample, and determining the CD subtype status of the subject based upon the expression profile, wherein an increased level of expression in the one or more genes as compared to a reference expression profile indicates status of CD-PBmu subtype as distinguished from a non-CD-PBmu subtype. (244) The method of embodiment 243, wherein the one or more genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 genes. (245) The method of embodiment 244, wherein the one or more genes comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or all of the genes in Tables 1A-1B. (246) The method of any of embodiments 67-98, 130-167, or 203-245, wherein the one or more genes comprises ADH4. (247) The method of any of embodiments 67-98, 130-167, or 203-246, wherein the one or more genes comprises ALG1L. (248) The method of any of embodiments 67-98, 130-167, or 203-247, wherein the one or more genes comprises BCDIN3D. (249) The method of any of embodiments 67-98, 130-167, or 203-248, wherein the one or more genes comprises C1orf106. (250) The method of any of embodiments 67-98, 130-167, or 203-249, wherein the one or more genes comprises C2. (251) The method of any of embodiments 67-98, 130-167, or 203-250, wherein the one or more genes comprises CCDC144NL. (252) The method of any of embodiments 67-98, 130-167, or 203-251, wherein the one or more genes comprises CEACAM5. (253) The method of any of embodiments 67-98, 130-167, or 203-252, wherein the one or more genes comprises CTAGE8. (254) The method of any of embodiments 67-98, 130-167, or 203-253, wherein the one or more genes comprises DDX11L2. (255) The method of any of embodiments 67-98, 130-167, or 203-254, wherein the one or more genes comprises DPPA4. (256) The method of any of embodiments 67-98, 130-167, or 203-255, wherein the one or more genes comprises DUSP19. (257) The method of any of embodiments 67-98, 130-167, or 203-256, wherein the one or more genes comprises FGB. (258) The method of any of embodiments 67-98, 130-167, or 203-257, wherein the one or more genes comprises GP2. (259) The method of any of embodiments 67-98, 130-167, or 203-258, wherein the one or more genes comprises GYPE. (260) The method of any of embodiments 67-98, 130-167, or 203-259, wherein the one or more genes comprises HSD3B7. (261) The method of any of embodiments 67-98, 130-167, or 203-260, wherein the one or more genes comprises HUNK. (262) The method of any of embodiments 67-98, 130-167, or 203-261, wherein the one or more genes comprises JAM2. (263) The method of any of embodiments 67-98, 130-167, or 203-262, wherein the one or more genes comprises KCNE3. (264) The method of any of embodiments 67-98, 130-167, or 203-263, wherein the one or more genes comprises KRT42P. (265) The method of any of embodiments 67-98, 130-167, or 203-264, wherein the one or more genes comprises LYZ. (266) The method of any of embodiments 67-98, 130-167, or 203-265, wherein the one or more genes comprises MLLT10P1. (267) The method of any of embodiments 67-98, 130-167, or 203-266, wherein the one or more genes comprises NAP1L6. (268) The method of any of embodiments 67-98, 130-167, or 203-267, wherein the one or more genes comprises NEURL3.

(269) The method of any of embodiments 67-98, 130-167, or 203-268, wherein the one or more genes comprises NPIPB9. (270) The method of any of embodiments 67-98, 130-167, or 203-269, wherein the one or more genes comprises PANK1. (271) The method of any of embodiments 67-98, 130-167, or 203-270, wherein the one or more genes comprises PKIB. (272) The method of any of embodiments 67-98, 130-167, or 203-271, wherein the one or more genes comprises RHOU. (273) The method of any of embodiments 67-98, 130-167, or 203-272, wherein the one or more genes comprises RPSAP9. (274) The method of any of embodiments 67-98, 130-167, or 203-273, wherein the one or more genes comprises SHCBP1. (275) The method of any of embodiments 67-98, 130-167, or 203-274, wherein the one or more genes comprises SIGLEC8. (276) The method of any of embodiments 67-98, 130-167, or 203-275, wherein the one or more genes comprises SLC15A2. (277) The method of any of embodiments 67-98, 130-167, or 203-276, wherein the one or more genes comprises SLC25A34. (278) The method of any of embodiments 67-98, 130-167, or 203-277, wherein the one or more genes comprises SLC6A20. (279) The method of any of embodiments 67-98, 130-167, or 203-278, wherein the one or more genes comprises SLC9B1. (280) The method of any of embodiments 67-98, 130-167, or 203-279, wherein the one or more genes comprises SYNPO2L. (281) The method of any of embodiments 67-98, 130-167, or 203-280, wherein the one or more genes comprises TDGF1. (282) The method of any of embodiments 67-98, 130-167, or 203-281, wherein the one or more genes comprises ZNF491. (283) The method of any of embodiments 67-98, 130-167, or 203-282, wherein the one or more genes comprises ZNF620. (284) The method of any of embodiments 67-98, 130-167, or 203-283, wherein the one or more genes comprises ZNF69. (285) The method of any of embodiments 67-98, 130-167, or 203-284, wherein the one or more genes comprises CXCL16. (286) The method of any of embodiments 67-98, 130-167, or 203-285, wherein the one or more genes comprises CD68. (287) The method of any of embodiments 67-98, 130-167, or 203-286, wherein the one or more genes comprises CD300E.

(288) The method of any one of embodiments 1-287, wherein the expression of at least one of the one or more genes in the biological sample is at least 2-fold greater than in the reference expression profile. (289) The method of any of embodiments 1-288, wherein the reference expression profile comprises expression levels of the one or more genes of one or more subjects who do not have IBD or have a PBT subtype of CD. (290) The method of any of embodiments 1-289, wherein detecting expression of the one or more genes comprises a RNA sequencing method. (291) The method of any of embodiments 1-290, wherein detecting expression of the one or more genes comprises a microarray method. (292) The method of any of embodiments 1-291, wherein detecting expression of the one or more genes comprises hybridization of a nucleic acid primer and/or probe to the biological sample, wherein the nucleic acid primer and/or probe comprises at least about 10 contiguous nucleobases of one of the one or more genes from Tables 1A-1B. (293) The method of any of embodiments 1-292, wherein the reference expression profile is stored in a database.

(294) The method of any of embodiments 1-293, further comprising treating the subject with a therapeutic agent. (295) The method of embodiment 294, wherein the therapeutic agent comprises a therapeutic of Table 20B; a protein, peptide, nucleic acid, or compound that targets a molecule of Tables 14, 15, 17A-17B, 20A; or a compound that targets a molecule in a pathway of one or more genes of Table 17B; or any combination thereof (296) The method of any of embodiments 1-295, provided the biological sample comprises a blood sample or is purified from a blood sample of the subject. (297) The method of any of embodiments 1-296, wherein the subject is less than 18 years of age. (298) The method of any of embodiments 1-297, wherein the subject is 18 years of age or older. (299) The method of any of embodiments 1-298, wherein the subject is not responsive to anti-TNFα therapy. (300) The method of any of embodiments 1-299, wherein the subject has or is susceptible to having stricturing disease. (301) The method of any of embodiments 1-300, wherein the subject has or is susceptible to having increased length of bowel resection.

(302) A method for processing or analyzing a biological sample from a subject, comprising: (a) obtaining the biological sample comprising gene expression products, wherein the subject has or is suspected of having Crohn's Disease (CD); (b) subjecting the biological sample to an assay by sequencing, array hybridization, and/or nucleic acid amplification to yield a data set including data corresponding to gene expression product levels; (c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and (d) electronically outputting a report that identifies the classification of the biological sample as positive or negative for the CD subtype. (303) The method of embodiment 302, wherein the sample is classified at an accuracy of at least about 90%, 91%, 92%, 93%, 94%, 95%, 96%, 97%, 98%, or 99%. (304) The method of embodiment 302 or embodiment 303, wherein the gene expression product comprises ribonucleic acid. (305) The method of any of embodiments 302-304, wherein the assay comprises using one or more of the following: microarray, sequencing, SAGE, blotting, reverse transcription, and quantitative polymerase chain reaction (PCR). (306) The method of any of embodiments 302-305, wherein the trained algorithm is trained with one or more datasets of gene expression product levels obtained from the plurality of training samples. (307) The method of any of embodiments 302-306, wherein the gene expression products comprise one or more genes from Tables 1A-1B.

(308) A composition comprising at least 10 but less than 100 contiguous nucleobases of a gene of Tables 1A-1B or its complement, and a detectable label.

(309) A panel of biomarker nucleic acids comprising at least 10 but less than 100 contiguous nucleobases of a plurality of genes, the plurality of genes comprising two or more genes from Tables 1A-1B.

(310) A composition comprising an agent that modulates expression and/or activity of a molecule in a pathway of one or more genes selected from Tables 1A-1B.

(311) A method comprising treating a subject with a therapeutic agent that targets a molecule in a pathway of one or more genes selected from Tables 1A-1B, wherein the subject is determined to have a CD-PBmu subtype as described in any of embodiments 243-301.

(312) The method of embodiment 310 or 311, wherein the agent comprises a peptide, nucleic acid, compound, or a combination thereof.

(313) A method comprising determining an increase or decrease in expression of a gene effectuated by a therapeutic agent in a subject, the method comprising detecting expression of the gene after administration of the therapeutic agent to the subject, wherein the gene is selected from Tables 1A-1B. (314) The method of embodiment 313, wherein the therapeutic agent comprises a therapeutic of Table 20B; a protein, peptide, nucleic acid, or compound that targets a molecule of Tables 14, 15, 17A-17B, 20A; or a compound that targets a molecule in a pathway of one or more genes of Table 17B; or any combination thereof. (315) The method of embodiment 313 or embodiment 314, wherein the expression is detected using the method of any of embodiments 243-301.

(316) A method comprising administering to the subject a kinase inhibitor, wherein the subject is determined to have a CD-PBmu subtype as described in any of embodiments 243-301.

(317) The method of any of embodiments 243-301, further comprising administering to the subject a kinase inhibitor.

(318) The method of embodiment 316 or embodiment 317, wherein the kinase target of the kinase inhibitor is a kinase described herein. (319) The method of embodiment 317 or embodiment 318, wherein the kinase target of the kinase inhibitor comprises a kinase of FIG. 6. (320) The method of embodiment 317 or embodiment 318, wherein the kinase target of the kinase inhibitor comprises a kinase of FIG. 7C. (321) The method of embodiment 317 or embodiment 318, wherein the kinase target of the kinase inhibitor comprises a kinase of FIG. 7D.

(322) A method comprising administering to the subject a modulator of a molecule of Table 14, wherein the subject is determined to have a CD-PBmu subtype as described in any of embodiments 243-301. (323) The method of any of embodiments 243-301, further comprising administering to the subject a modulator of a molecule of Table 14.

(324) A method comprising administering to the subject a modulator of a molecule of Table 15, wherein the subject is determined to have a CD-PBmu subtype as described in any of embodiments 243-301. (325) The method of any of embodiments 243-301, further comprising administering to the subject a modulator of a molecule of Table 15.

(326) A method comprising administering to the subject a modulator of a molecule of Table 17A, wherein the subject is determined to have a CD-PBmu subtype as described in any of embodiments 243-301. (327) The method of any of embodiments 243-301, further comprising administering to the subject a modulator of a molecule of Table 17A.

(328) A method comprising administering to the subject a modulator of a molecule of Table 17B, wherein the subject is determined to have a CD-PBmu subtype as described in any of embodiments 243-301. (329) The method of any of embodiments 243-301, further comprising administering to the subject a modulator of a molecule of Table 17B.

(330) A method comprising administering to the subject a modulator of a molecule of Table 20A, wherein the subject is determined to have a CD-PBmu subtype as described in any of embodiments 243-301. (331) The method of any of embodiments 243-301, further comprising administering to the subject a modulator of a molecule of Table 20A.

(332) A method comprising administering to the subject a modulator of a compound that targets a molecule in a pathway of one or more genes of Table 17B, wherein the subject is determined to have a CD-PBmu subtype as described in any of embodiments 243-301. (333) The method of any of embodiments 243-301, further comprising administering to the subject a modulator of a compound that targets a molecule in a pathway of one or more genes of Table 17B.

(334) A method comprising administering to the subject a therapeutic of Table 20B, wherein the subject is determined to have a CD-PBmu subtype as described in any of embodiments 243-301. (335) The method of any of embodiments 243-301, further comprising administering to the subject a therapeutic of Table 20B.

(336) A method comprising administering to the subject an anti-TL1A antibody, wherein the subject is determined to have a CD-PBmu subtype as described in any of embodiments 243-301. (337) The method of any of embodiments 243-301, further comprising administering to the subject a an anti-TL1A antibody. (338) The method of embodiment 336 or embodiment 337, wherein the anti-TL1A antibody comprises CDRs comprising SEQ ID NOS: 346-351.

### EXAMPLES

While preferred embodiments have been shown and described herein, it will be obvious to those skilled in the art that such embodiments are provided by way of example only. Numerous variations, changes, and substitutions will now occur to those skilled in the art without departing from the embodiments provided. It should be understood that various alternatives to the embodiments described herein may be employed.

### Example 1: Blood Based Pre-Surgical Transcriptomic Signature

A Treatment-Resistant CD Population Characterized by Mucosal-Like T Cells Circulating in the Periphery

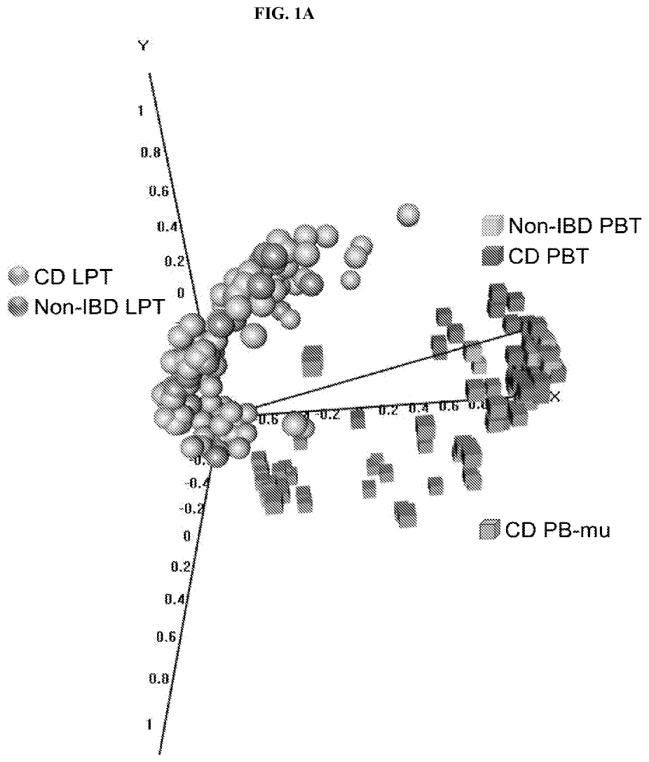

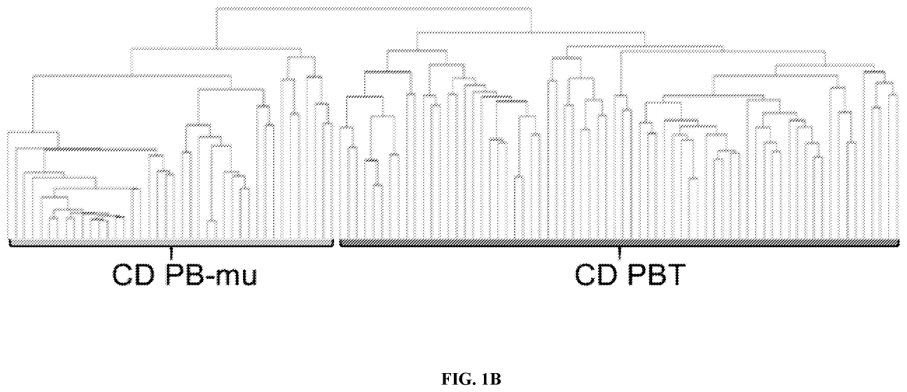

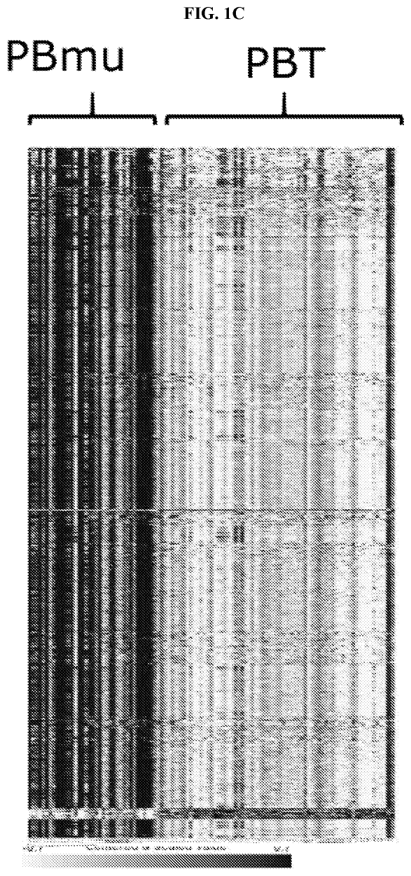

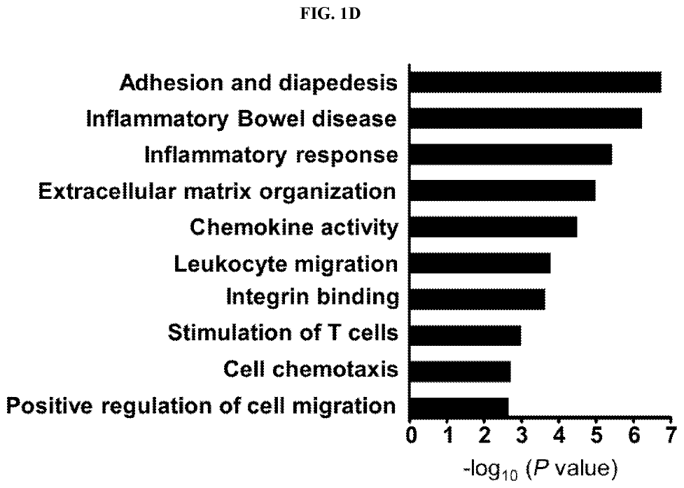

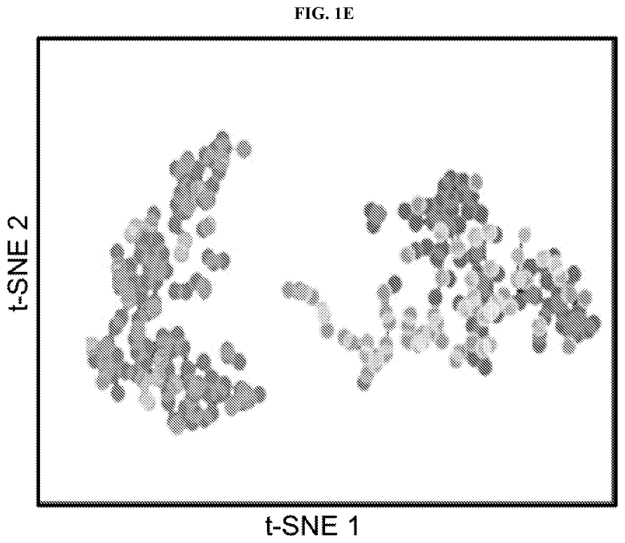

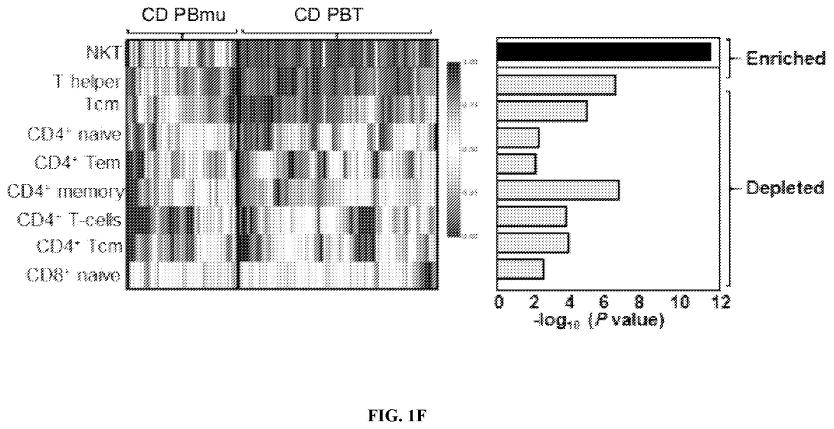

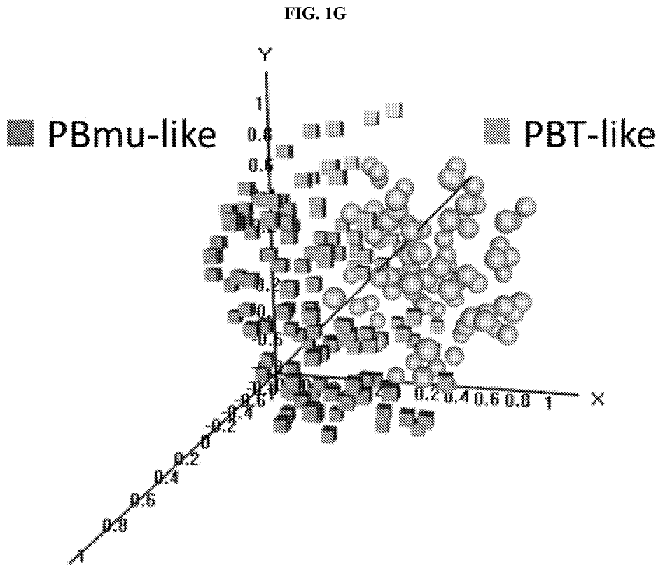

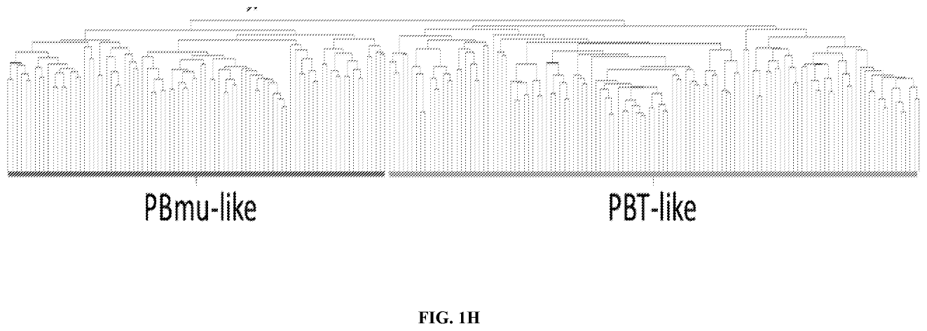

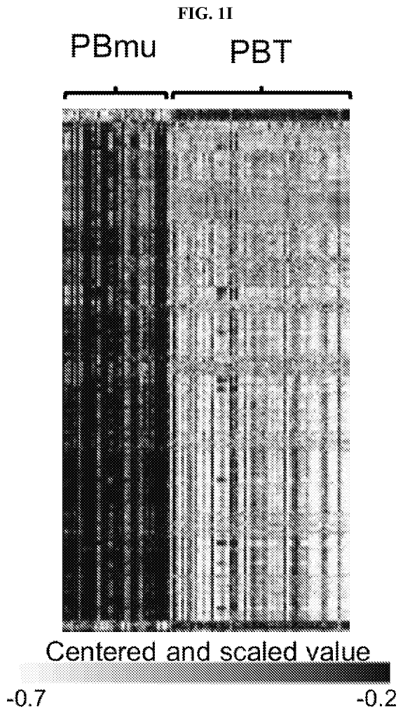

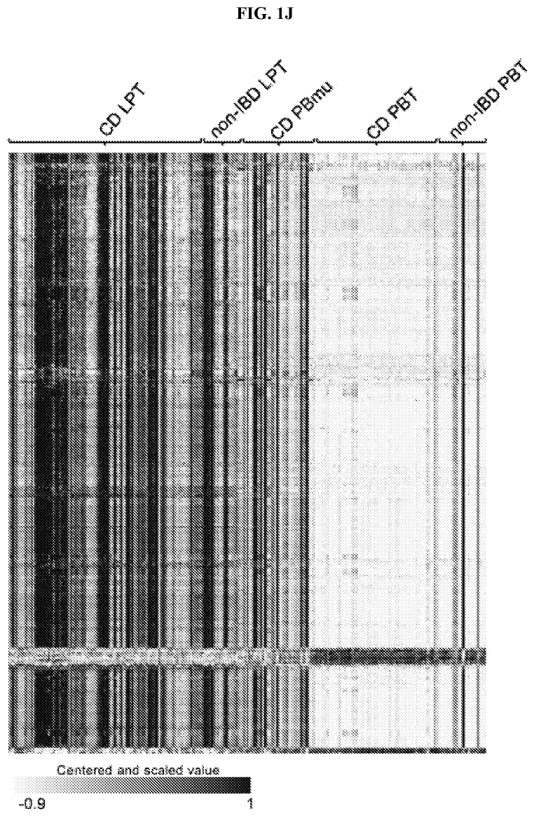

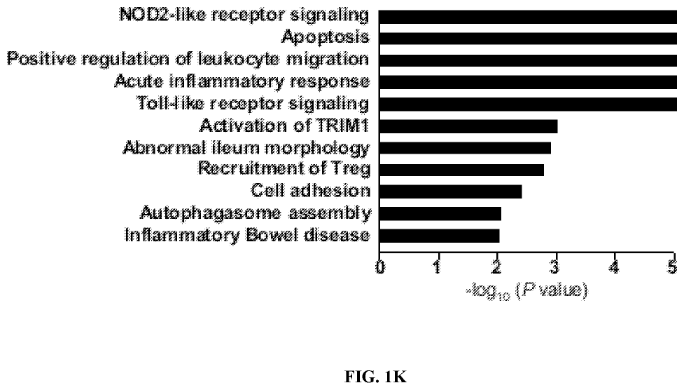

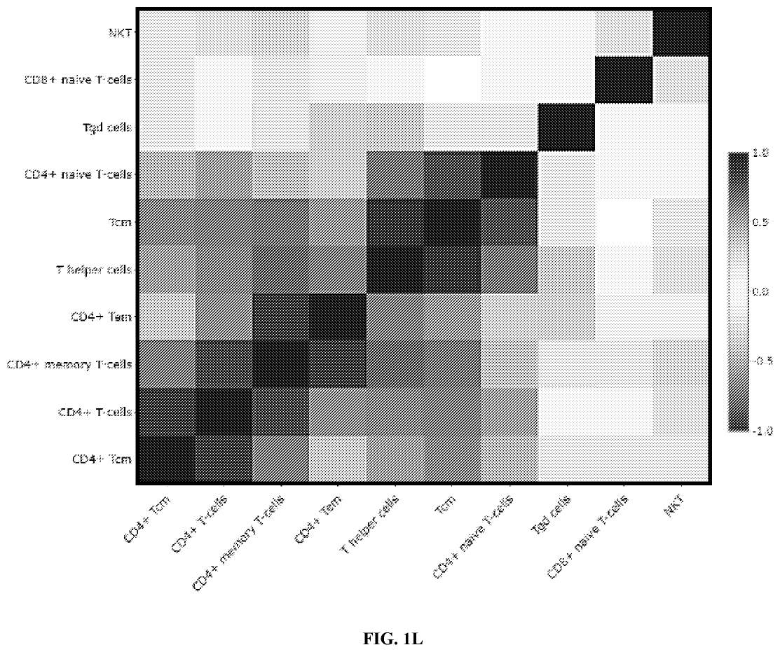

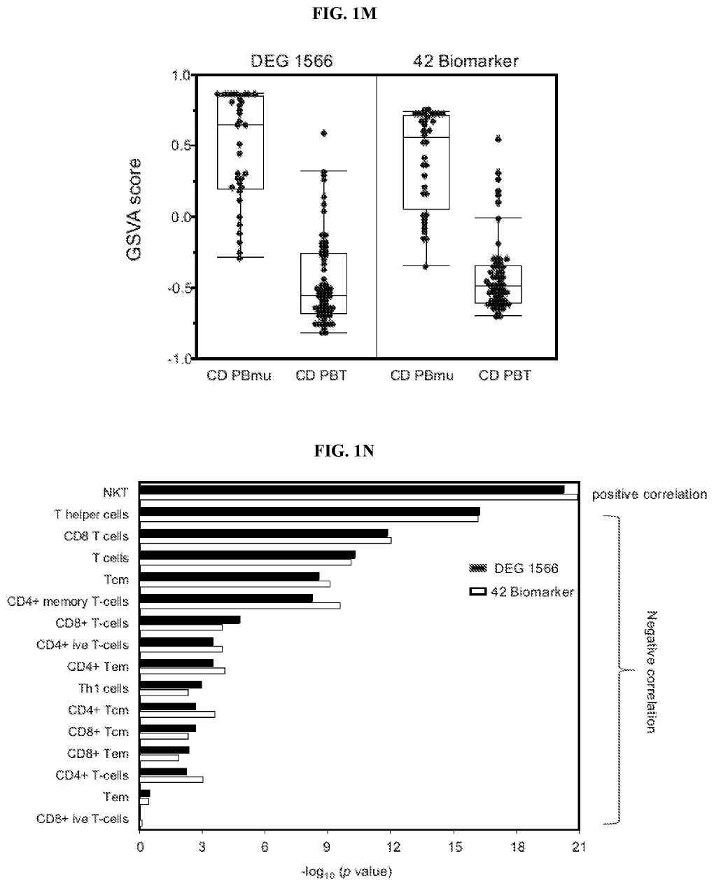

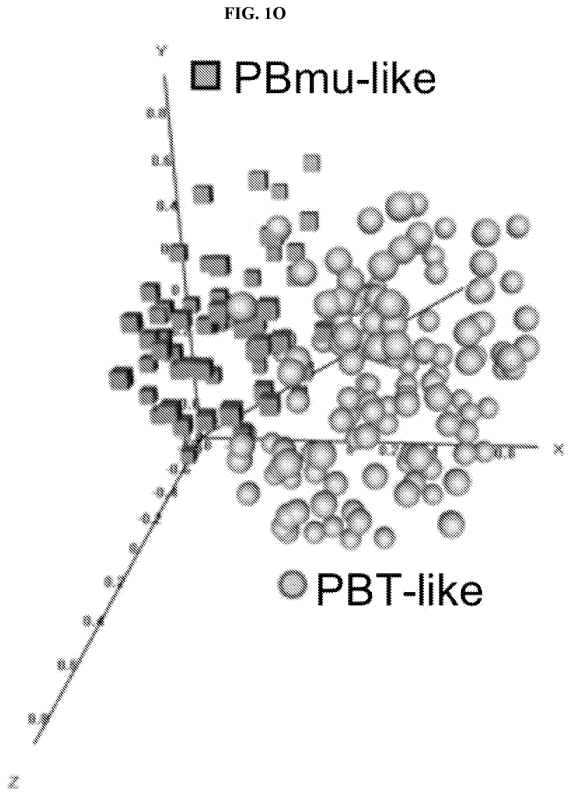

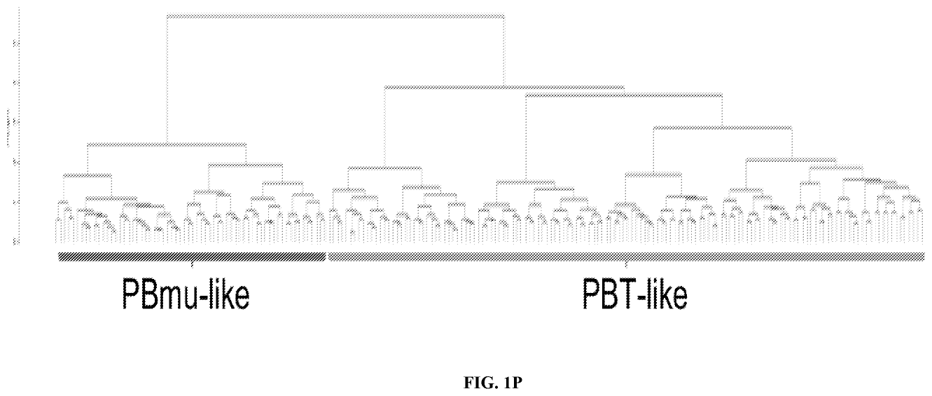

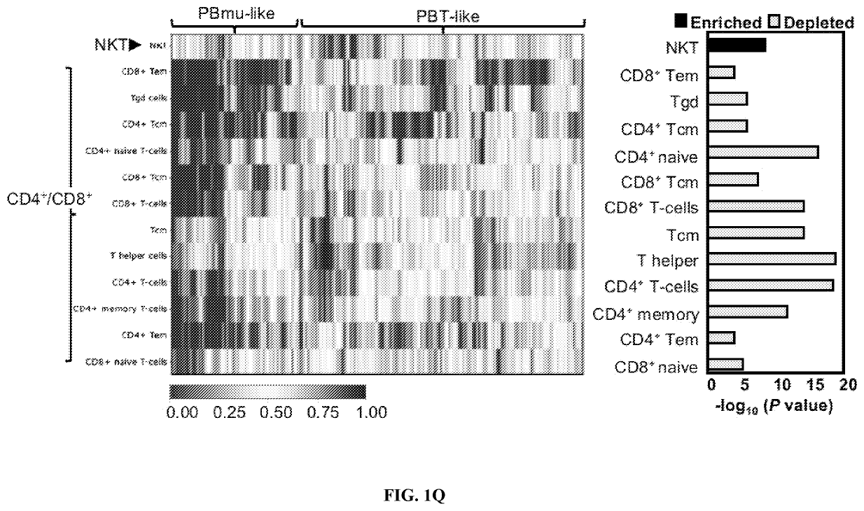

This experiment was performed to identify molecular pathways underlying T cell transcriptomic signatures in treatment-resistant CD patients who required surgical intervention for disease management. Purified CD3+ T cells were isolated from matched paired samples from peripheral blood and mucosal specimens from 100 CD patients and 17 control non-IBD individuals at the time of surgery. Principal component analysis of gene expression distinguished between lamina propria mucosa-derived (mucosal, CD LPT) T cells and those in the periphery (FIG. 1A). Among mucosal T cells, the expression profile of CD patients and non-IBD subjects was interspersed. In contrast, among peripheral T cells, two distinct CD transcriptomic signatures were observed. One expression signature, designated CD-PBT (63%), clustered tightly with non-IBD subjects. A second peripheral expression signature was shifted towards the mucosal T cell signature, and was designated CD-PBmu(cosal) (37%) (FIGS. 1A-1B).

The subtype classification (≥90%) was confirmed using multiple statistical techniques including Bayesian nearest neighbor predictor, support-vector machine and diagonal linear discriminant analysis (Table 13A). 1944 genes were identified with at least two-fold differential expression between CD-PBmu and CD-PBT subsets (p value <0.001) (FIG. 1C). Among them, >90% of genes were over-expressed in the CD-PBmu subtype. Pathway analysis indicated that the CD-PBmu differentially expressed genes (DEG) were enriched in pathways associated with T cell activation, leukocyte adhesion and migration, and integrin binding features (FIG. 1D). Without being bound by theory, these mucosal-like features suggest that CD-PBmu might represent recent mucosal emigrants.

TABLE 13A
Performance of CD-PBmu vs CD-PBT classifiers
during cross-validation

| | % Correct | Sensi- | Speci- | | |
|---|---|---|---|---|---|
| Classifier | Classification | tivity | ficity | PPV | NPV |
| Compound Covariate | 89 | 0.75 | 0.96 | 0.92 | 0.87 |
| Predictor | | | | | |
| Diagonal Linear | 90 | 0.77 | 0.97 | 0.94 | 0.88 |
| Discriminant Analysis | | | | | |
| 1-Nearest Neighbor | 93 | 0.82 | 0.95 | 0.91 | 0.90 |
| 3-Nearest Neighbor | 91 | 0.78 | 0.95 | 0.90 | 0.88 |
| Nearest Centroid | 86 | 0.71 | 0.94 | 0.87 | 0.85 |
| Support Vector | 93 | 0.84 | 0.94 | 0.89 | 0.91 |
| Machine | | | | | |
| Bayesian Compound | 92 | 0.57 | 0.81 | 0.64 | 0.76 |
| Covariate | | | | | |

Positive Predictive Value (PPV), Negative Predictive Value (NPV)

The subtype classification was further validated as shown in Tables 21A-21B. 1566 genes with at least two-fold differential expression between CD-PBmu and CD-PBT subtypes were identified (p value <0.001, FDR <0.002) (FIGS. 1I-1J). Pathway analysis indicated that the CD-PBmu differentially expressed genes (DEG) were enriched in pathways associated with T cell activation, leukocyte adhesion and migration, and integrin binding features (FIG. 1K).

TABLE 21A
CD-PBmu vs CD-PBT classifiers during cross-validation

| | % correct | Sensi- | Speci- | | |
|---|---|---|---|---|---|
| Classifier: | classification | tivity | ficity | PPV | NPV |
| Compound Covariate | 85 | 0.61 | 0.98 | 0.95 | 0.82 |
| Predictor | | | | | |
| Diagonal Linear | 86 | 0.62 | 0.98 | 0.95 | 0.83 |
| Discriminant Analysis | | | | | |
| 1-Nearest Neighbor | 93 | 0.83 | 0.94 | 0.89 | 0.91 |
| 3-Nearest Neighbor | 92 | 0.82 | 0.94 | 0.89 | 0.90 |
| Nearest Centroid | 85 | 0.61 | 0.98 | 0.94 | 0.82 |
| Support Vector | 94 | 0.84 | 0.94 | 0.89 | 0.94 |
| Machine | | | | | |
| Bayesian Compound | 89 | 0.46 | 0.86 | 0.63 | 0.74 |
| Covariate | | | | | |

TABLE 21B
CD-PBmu transcriptomic signature in classifying
of whole blood validation cohort (GSE100833)
into PBmu-like and PBT-like patient subtypes

| | % correct | Sensi- | Speci- | | |
|---|---|---|---|---|---|
| Classifier: | classification | tivity | ficity | PPV | NPV |
| Compound Covariate | 90 | 0.77 | 0.95 | 0.87 | 0.90 |
| Predictor | | | | | |
| Diagonal Linear | 89 | 0.75 | 0.94 | 0.86 | 0.89 |
| Discriminant Analysis | | | | | |
| 1-Nearest Neighbor | 94 | 0.82 | 0.95 | 0.88 | 0.92 |
| 3-Nearest Neighbor | 92 | 0.78 | 0.95 | 0.87 | 0.90 |
| Nearest Centroid | 91 | 0.80 | 0.95 | 0.88 | 0.91 |
| Support Vector | 92 | 0.82 | 0.94 | 0.86 | 0.92 |
| Machine | | | | | |
| Bayesian Compound | 95 | 0.65 | 0.83 | 0.63 | 0.84 |
| Covariate | | | | | |

Next, it was assessed whether the transcriptomic signature stratifying CD-PBmu vs CD-PBT subtype was associated with clinically relevant disease markers that may reflect a larger burden of mucosal inflammatory disease prior to surgery. To minimize variability in clinical assessment, patients were evaluated, and surgical samples collected from resections performed by a single surgical provider. No significant differences were noted between the demographics of the CD-PBmu compared to CD-PBT patient populations (Table 22). Moreover, there were no significant associations in disease location or behavior, length or location of intestinal resection or pre-operative medications. Additionally, both a pre-operative disease severity score based on a weighted disease index and surgical pathological severity score based on the depth and extent of inflammation in the resected segment were calculated. These severity scores likewise failed to stratify the peripheral CD-PBmu vs CD-PBT subtypes suggesting that transcriptomic signature was not merely reflective of a global enhanced inflammation in the CD-PBmu subtype.

TABLE 22
Demographics of CD-PBmu and CD-PBT patient populations

| | | | |
|---|---|---|---|
| Patient characteristics at | | | |
| time of surgery | Total | PBmu | PBT |
| Number of patients | n = 100 | n = 36 | n = 64 |

Variable n/total (%)
Gender

| Female | 41/100 | (41) | 11/36 | (31) | 30/64 | (47) |
|---|---|---|---|---|---|---|
| Age at diagnosis (median | 24 | (16-32) | 25 | (18-35) | 23 | (16-32) |
| and IQR), yr. | | | | | | |
| Montreal classification | | | | | | |
| ≤16 years (A1) | 25/97 | (26) | 6/35 | (17) | 19/62 | (31) |
| 17-40 years (A2) | 55/97 | (57) | 23/35 | (66) | 32/62 | (52) |
| >40 years (A3) | 17/97 | (18) | 6/35 | (17) | 11/62 | (18) |
| Disease duration (median | 7 | (3-14) | 8 | (3-16) | 7 | (2-13) |
| and IQR), yrs. | | | | | | |
| Age at surgery (median and | 35 | (24-51) | 37 | (27-53) | 35 | (24-49) |
| IQR), yr. | | | | | | |
| Family history of IBD | 34/97 | (35) | 10/35 | (29) | 24/62 | (39) |
| First resection | 63/86 | (73) | 22/29 | (75) | 41/57 | (72) |
| Anemia at surgery | 37/73 | (51) | 11/24 | (46) | 26/49 | (53) |
| Elevated CRP at surgery | 44/74 | (60) | 15/27 | (44) | 29/47 | (62) |
| pre-op overall severity | 15 | (12-22) | 19 | (14-24) | 14 | (10-20) |
| index (median and IQR) | | | | | | |
| Pre-operative treatment | | | | | | |
| history | | | | | | |
| Steroids | 70/91 | (77) | 27/31 | (87) | 43/60 | (71) |
| anti-TNF | 58/87 | (67) | 16/27 | (59) | 42/60 | (70) |
| immunomodulators | 62/77 | (81) | 21/25 | (84) | 41/52 | (79) |
| CD disease location | | | | | | |
| L1 (ileal) | 6/97 | (6) | 4/34 | (11) | 2/58 | (3) |
| L2 (colonic) | 11/97 | (11) | 3/34 | (9) | 8/58 | (13) |
| L3 (ileocolonic) | 80/97 | (83) | 28/34 | (80) | 52/58 | (84) |
| CD Disease Behavior | | | | | | |
| B1 (non-stricturing, non- | 13/89 | (15) | 4/29 | (14) | 9/60 | (15) |
| penetrating) | | | | | | |
| B2 (isolated stricturing) | 48/89 | (54) | 16/29 | (55) | 32/60 | (53) |
| B3 (penetrating or | 28/89 | (31) | 9/29 | (31) | 19/60 | (32) |
| stricturing and penetrating) | | | | | | |
| Perianal disease | 24/73 | (33) | 11/24 | (46) | 13/49 | (25) |
| Resected Disease location | | | | | | |
| Small bowel | 6/99 | (6) | 3/36 | (8) | 3/63 | (5) |
| Ileocecal/Ileocolic | 66/99 | (67) | 21/36 | (58) | 45/63 | (71) |
| Colon | 27/99 | (27) | 12/36 | (34) | 15/63 | (24) |
| Resected bowel length | 33 | (22-56) | 38 | (24-61) | 33 | (22-51) |
| (median and IQR), cm. | | | | | | |
| Granuloma in resected | 26/77 | (34) | 8/25 | (32) | 18/52 | (35) |
| segment | | | | | | |
| Microscopic disease at | 17/76 | (22) | 8/25 | (32) | 9/51 | (18) |
| margins | | | | | | |
| Severity Score | | | | | | |
| pre-op overall severity | 15 | (12-22) | 19 | (14-24) | 14 | (10-20) |
| index (median and IQR) | | | | | | |

Pathology based severity score - deep ulcers, strictures, fistula, fissures

| mild activity (1-2.9 cm) | 19/94 | (20) | 5/34 | (15) | 14/60 | (23) |
|---|---|---|---|---|---|---|
| moderate activity (3-5 cm) | 11/94 | (11) | 4/34 | (12) | 7/60 | (12) |
| severe activity (>5 cm, deep | 64/94 | (68) | 25/34 | (74) | 39/60 | (65) |
| fissures | | | | | | |
| post-op endoscopic | | | | | | |
| recurrence (Rutgeerts | | | | | | |
| score) | | | | | | |
| Time between resection and | 10 | (7-23) | 10 | (6-20) | 10 | (7-25) |
| colonoscopy (median and | | | | | | |
| IQR), mo | | | | | | |
| I0 | 20/75 | (27) | 7/26 | (27) | 13/49 | (27) |
| I1 | 23/75 | (31) | 9/26 | (35) | 14/49 | (29) |
| I2 | 9/75 | (12) | 2/26 | (8) | 7/49 | (14) |
| I3 | 12/75 | (16) | 3/26 | (12) | 9/49 | (18) |
| I4 | 1/75 | (1) | 1/26 | (4) | 0/49 | (0) |
| no post-op endoscopy | 10/75 | (13) | 4/26 | (15) | 6/49 | (12) |
| Time between surgical and | 7 | (4-10) | 6 | (3-9) | 7 | (4-11) |
| post-op blood collection | | | | | | |
| (median and IQR), mo | | | | | | |
| post-op prophylactic | | | | | | |
| medication | | | | | | |
| Immunosuppressants | 32/63 | (51) | 10/22 | (46) | 22/41 | (54) |
| anti-TNF | 39/64 | (61) | 10/22 | (46) | 29/42 | (69) |

The Imputed Composition of Peripheral T Cell Subsets is Altered in CD-PBmu

CD3+ T cells are a heterogeneous population with a mosaic of naïve, activated, memory, and effector T cell traits defined by their cell surface markers and immune response. Alteration in the abundance of individual subsets can be quantified from RNA sequencing data using bioinformatic approaches. Experiments were designed to determine whether the distinct transcriptomic signatures observed in the CD-PBmu vs CD-PBT subtypes may result from an underlying alteration in peripheral T cell subset composition. Individual immune cell enrichment scores were calculated and a t-SNE analysis was applied. As seen in FIG. 1E, the t-SNE cell signature enrichment plot mimics that observed for the gene expression (FIG. 1A) with distinct clustering of the CD-PBmu vs CD-PBT subtypes. Comparison of CD-PBmu to CD-PBT subtype demonstrated inferred enrichment for NKT cells and depletion of TH1 and CD4+ and CD8+ memory and naïve cell subsets (FIG. 1F). The enrichment scores do not infer percentage comparison across cell types i.e. enrichment of NKT need not correlate with depletion of CD4+/CD8+ cells. Indeed, there was no significant correlation noted between the NKT and CD4+/CD8+ T cell subset enrichment scores (FIG. 1L). Likewise, a gene set variation analysis (GSVA) evaluating the enrichment of the differentially expressed gene set across all samples demonstrated significant correlation with T cell subset enrichment scores (FIGS. 1M-1N). To further confirm the deconvolution analysis, CD-PBmu and CD-PBT were compared using the Ingenuity analysis match metadata evaluator method. Differential gene expression and upstream regulatory pathways were observed that has previously been identified when comparing NKT cell to CD4+ T cell subsets (Table 13B), supporting these findings by deconvolution of the CD3+ T cell composition.

TABLE 13B
Concordance of CD-PBmu signature similarity matching gene expression
and upstream regulatory pathways associated when comparing NKT
cell to CD4 T cell subsets (Geo accession: GSE24759).

| | Overall | Overall | UR | DE | UR | DE |
|---|---|---|---|---|---|---|
| Comparison | p-value | z-score | (p-value) | (p-value) | (z-score) | (z-score) |
| NK T cell vs naïve | 12.47 | 21.38 | 7.12E−06 | 9.38E−20 | 35.04 | 50.49 |
| CD8+ T cell | | | | | | |
| NK T cell vs naïve | 8.55 | 19.2 | 5.41E−08 | 9.76E−09 | 39.74 | 37.05 |
| CD4+ T cell | | | | | | |
| NK T cell vs CD4+ | 3.46 | 8.11 | 1.30E−05 | 0.04 | 32.44 | |
| effector memory T | | | | | | |
| cell | | | | | | |
| NK T cell vs CD4+ | 2.63 | 7.4 | 1.31E−04 | | 29.62 | |
| central memory T cell | | | | | | |
| NK T cell vs CD8+ | 1.4 | | 0.03 | | | |
| central memory T cell | | | | | | |

The Peripheral T Cell Subset Composition in CD-PBmu is Associated with Distinct Clinical and Serological Characteristics of Disease Severity

The impact of altered gene expression and T cell subset composition on clinical characteristics of disease activity was assessed using 1566 Differentially Expressed Genes. A summary is shown in Table 13C.

TABLE 13C
T Cell Subset composition and clinical associations using 1944 Differentially Expressed Genes

| | |
|---|---|
| NKT cell enrichment in CD-PBmu vs CD-PBT | p = 5E−13 |
| NKT cell enrichment in CD-PBmu, but not CD-PBT, is associated with | p = 4.7E−02 |
| stricturing disease | |
| NKT cell enrichment in CD-PBmu, but not CD-PBT, correlated with | p = 3.3E−02 |
| ASCA serological response levels | |
| Decreased CD4+/CD8+ T cell subsets in CD-PBmu vs CD-PBT | p = 6.1E−03 − 1.7E−07 |
| Decreased CD4+ memory T cell is associated with increased length of | p = 1.2E−02 |
| bowel resection | |
| Serological quartile sum scores in CD-PBmu, but not CD-PBT, are | p = 2.9E−02 |
| associated with increased length of bowel resection | |
| Decreased CD4/CD8 memory T cell is associated with post-op | p = 3.3E−02 |
| recurrence in PBmu | |
| Attenuated gene expression in CD-PBmu, but not in CD-PBT, | 900 transcripts, p = 9.9E−04, |
| following surgery | FDR < 0.01, fold > 1.5 |

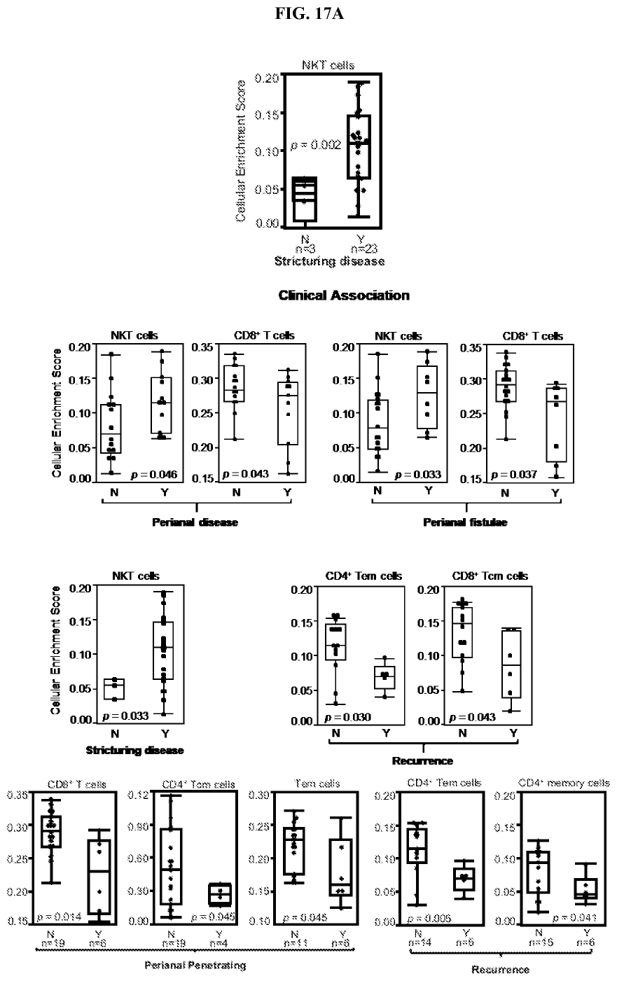

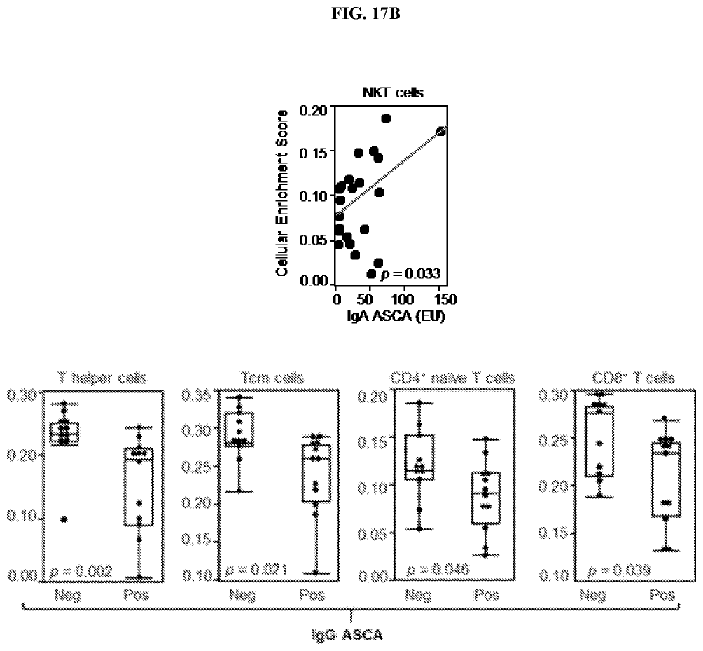

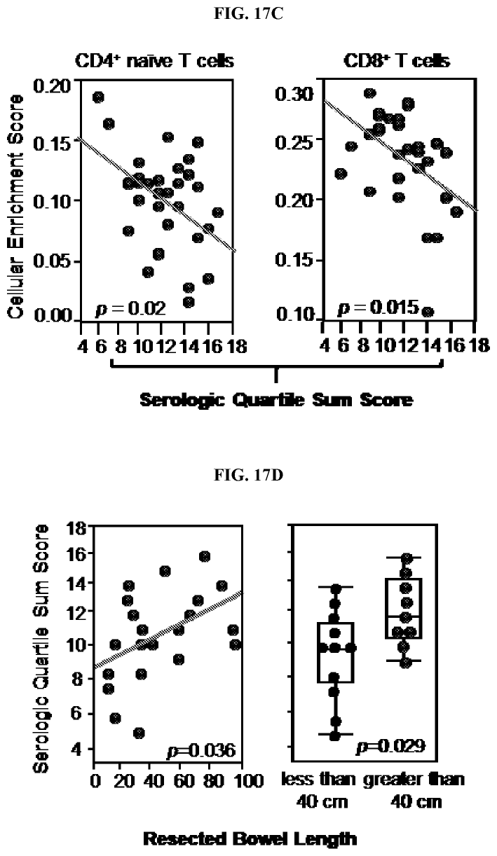

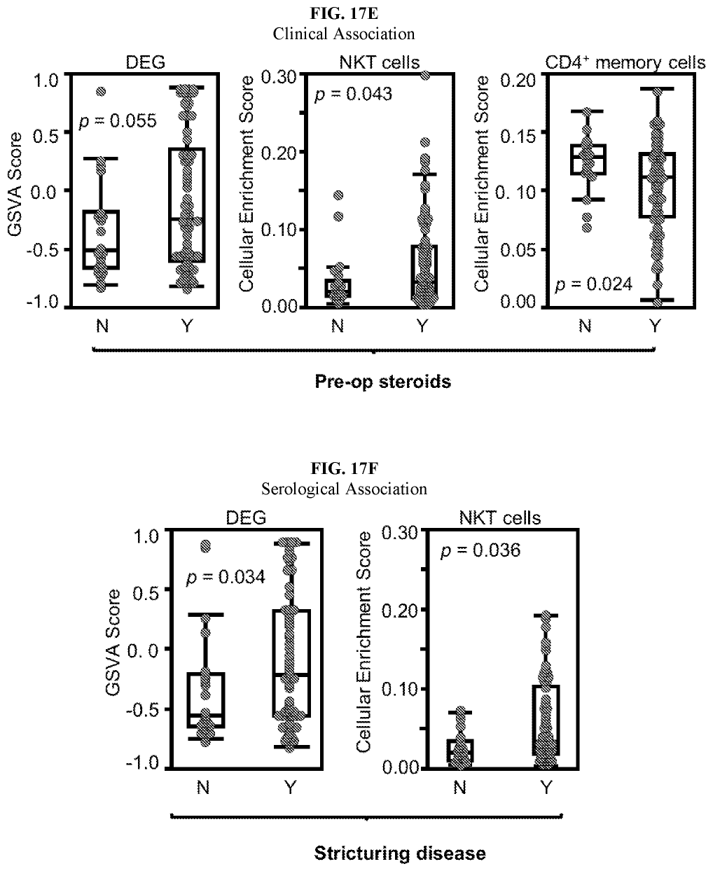

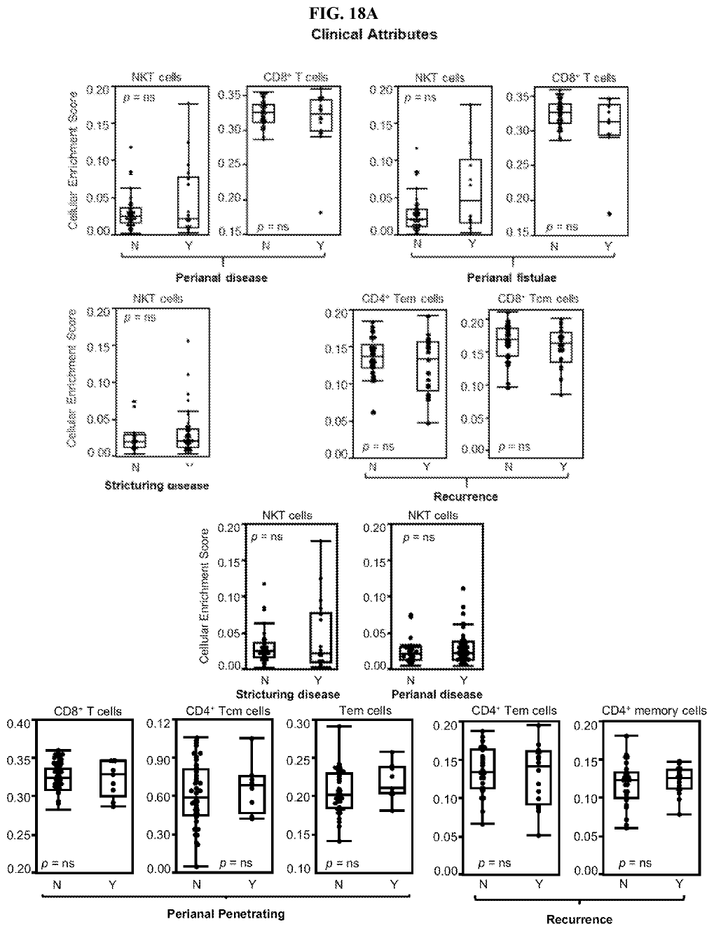

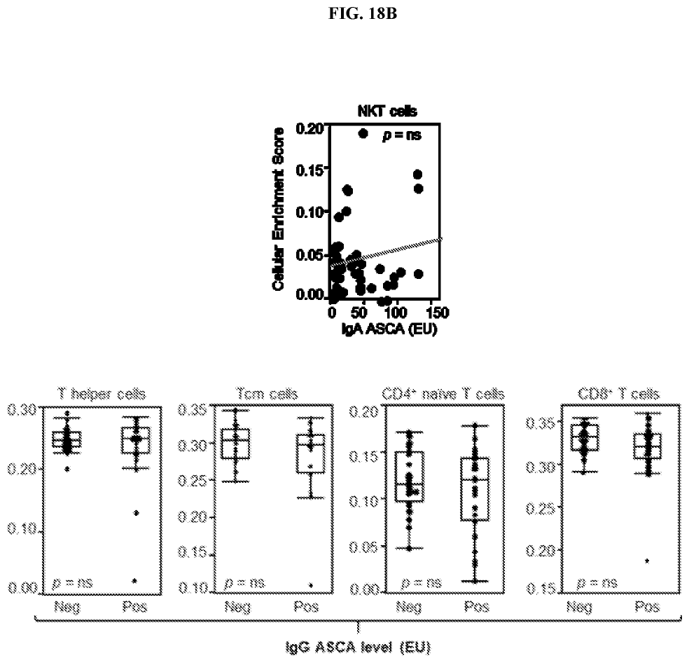

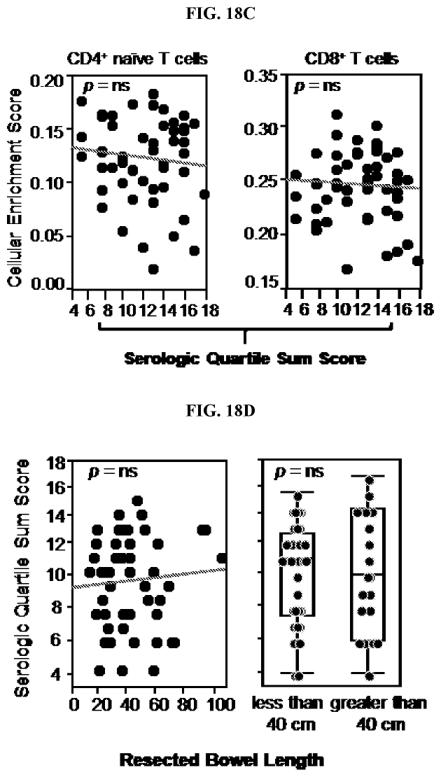

The impact of altered gene expression and T cell subset composition on clinical characteristics of disease activity was assessed using 1566 Differentially Expressed Genes. Pre-operative steroid use, stricturing disease and ANCA seropositivity were associated with GSVA differential gene expression scores and NKT and CD4+ memory T cell subset enrichment scores in a direction consistent with categorization of the CD-PBmu vs. PBT and reached statistical significance (FIGS. 17E-17F). Moreover, in the CD-PBmu (FIG. 17A), but not CD-PBT sub-type (FIG. 18A), NKT cell enrichment scores were associated with stricturing disease at time of surgery (FIG. 17A). The CD-PBmu vs CD-PBT subtype was significantly more likely to develop stricturing disease (Cochran Armitage trend test p=0.033). Presence of perianal disease at time of surgery and perianal fistula was likewise associated with enrichment in NKT cells, as well as, depletion of CD8+ T cells (FIG. 17A). Moreover, depletion of CD4+ and CD8+ T cell subsets observed in the CD-PBmu vs CD-PBT subtype was associated with perianal penetrating disease and post-operative endoscopic recurrence of disease (average interval for post-operative evaluation in both CD-PBmu and CD-PBT subtypes was 10 months) (FIG. 17A). Serologic responses to commensal bacteria and auto-antigens in CD patients such as ASCA, OmpC, I2 and anti-CBir1 have been associated with more severe clinical disease phenotypes and risk of complications. In particular, a high antibody response toward multiple microbial antigens is predictive of aggressive disease and risk for surgery. In the CD-PBmu, but not CD-PBT subtype, the NKT enrichment scores correlated with increased ASCA sero-positivity levels (FIG. 17B, FIG. 18B). Conversely, depletion of CD4+/CD8+ T cell subsets was associated with ASCA positivity. Moreover, in the CD-PBmu, but not CD-PBT subtype, depletion of CD4+ naïve and CD8+ T cells was associated with enhanced serological quartile sum scores of response (FIG. 17C, FIG. 18C) and enhanced serological quartile sum scores of response to multiple microbial antigens in CD-PBmu was associated with an increased length of resected intestine (FIG. 17D, FIG. 18D). These findings suggest that an altered T cell subset composition characterized by the CD-PBmu subtype may help sub-stratify disease within a patient population resistant to therapeutic intervention.

Validation of the CD-PBmu Transcriptomic Signature in an Independent Cohort

The reproducibility of the CD-PBmu transcriptomic signature to identify CD patient subtypes was tested using an independent treatment resistant cohort and dataset: gene expression in whole blood isolated from Crohn's disease patients who had failed treatment with anti TNF-alpha therapy. Hierarchical clustering using the initial gene set defining the CD-PBmu subtype (1944 transcripts) identified two distinct PBmu- and PBT-like clusters (FIGS. 1G-1H). Principal component analysis and differential gene expression distinguished between these groups, with approximately 33% of patients displaying a CD-PBmu-like expression pattern and an average classification performance of >90% (Table 13D).

Similarly, hierarchical clustering using the second gene set defining the CD-PBmu subtype (1566 transcripts) identified two distinct PBmu- and PBT-like clusters (FIGS. 1O-1P). Principal component analysis and differential gene expression distinguished between these groups, with approximately 31% of patients displaying a CD-PBmu-like expression pattern and an average classification performance of 92% (Table 21B). Moreover, cell type enrichment analysis revealed a similar inherent imbalance of T cells subsets with enrichment of NKT cells and depletion of CD4+/CD8+ subsets associated with the PBmu-like classification (FIG. 1Q). The imbalance in T cells subset composition was distinct for the CD-PBmu signature and was not observed when applying a random probe-gene set for clustering analysis (FIG. 1R).

TABLE 13D
Performance of CD-PBmu transcriptomic signature
in classifying of whole blood validation cohort
into PBmu-like and PBT-like patient subtypes.

| | % Correct | Sensi- | Speci- | | |
|---|---|---|---|---|---|
| Classifier | Classification | tivity | ficity | PPV | NPV |
| Compound Covariate | 93 | 0.81 | 0.96 | 0.89 | 0.93 |
| Predictor | | | | | |
| Diagonal Linear | 92 | 0.80 | 0.96 | 0.88 | 0.92 |
| Discriminant Analysis | | | | | |
| 1-Nearest Neighbor | 94 | 0.83 | 0.95 | 0.88 | 0.94 |
| 3-Nearest Neighbor | 94 | 0.82 | 0.95 | 0.87 | 0.93 |
| Nearest Centroid | 93 | 0.84 | 0.96 | 0.89 | 0.94 |
| Support Vector | 94 | 0.83 | 0.95 | 0.87 | 0.93 |
| Machine | | | | | |
| Bayesian Compound | 95 | 0.70 | 0.84 | 0.63 | 0.88 |
| Covariate | | | | | |

Positive Predictive Value (PPV), Negative Predictive Value (NPV)

The CD-PBmu Transcriptomic Signature Reverts to that Observed for CD-PBT Following Surgery

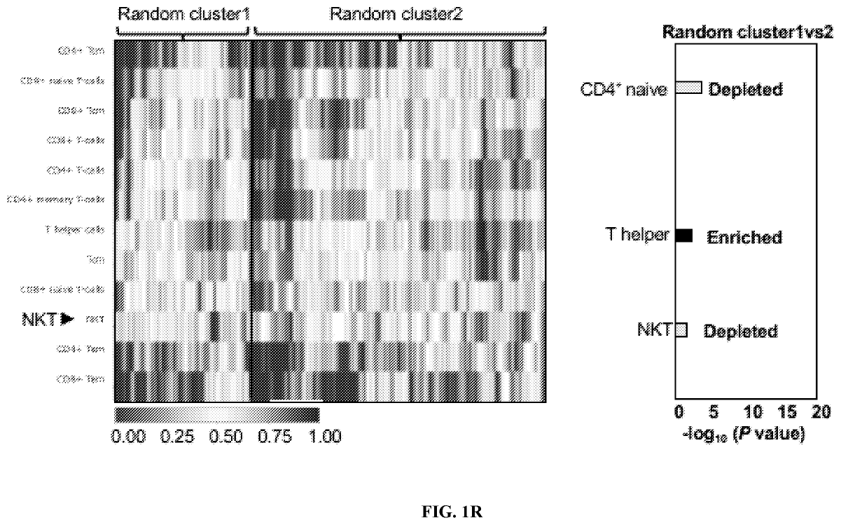

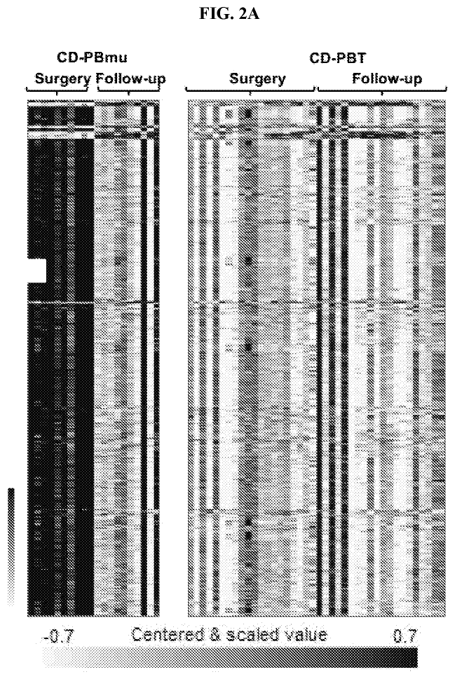

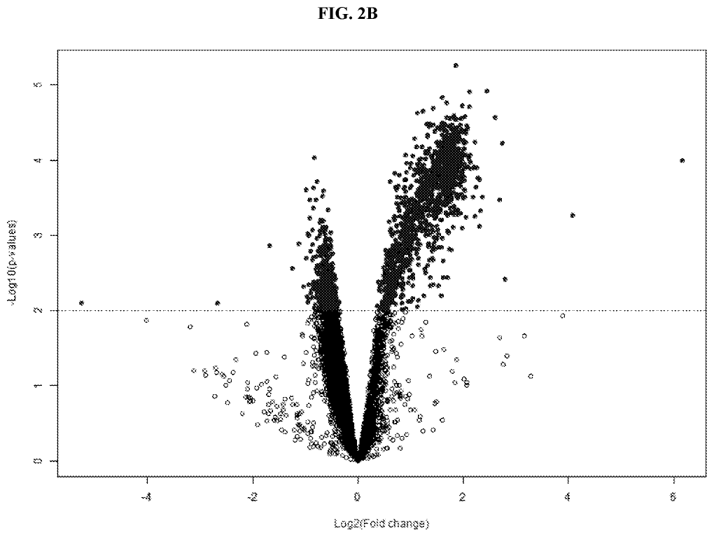

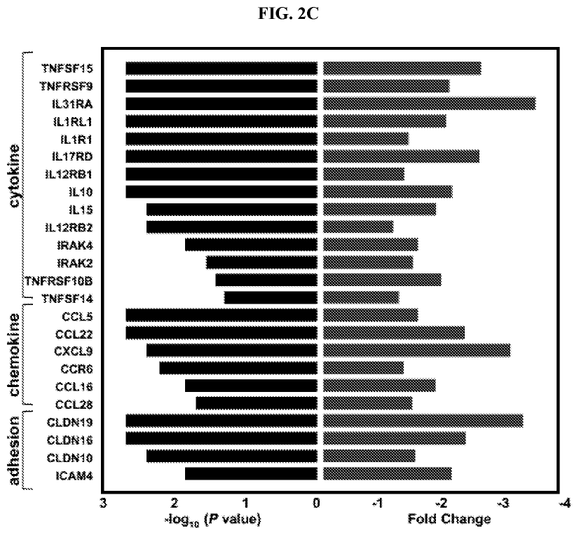

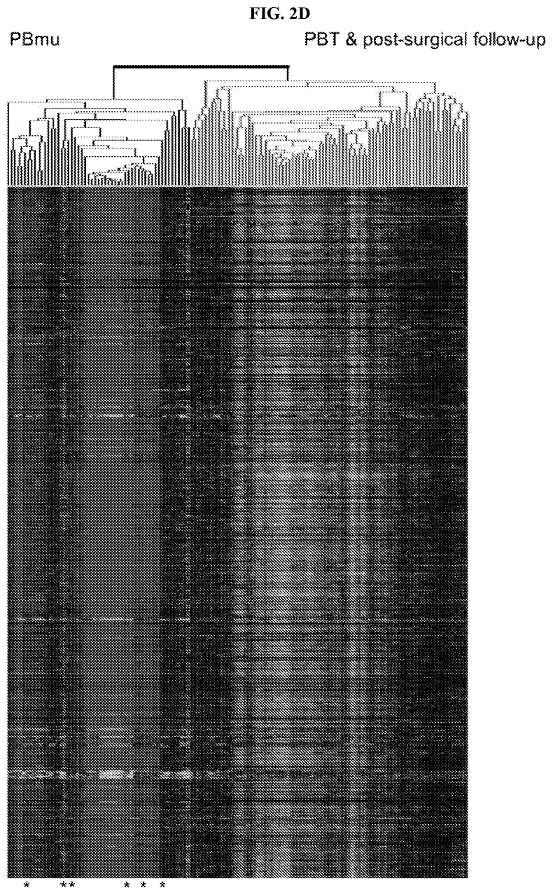

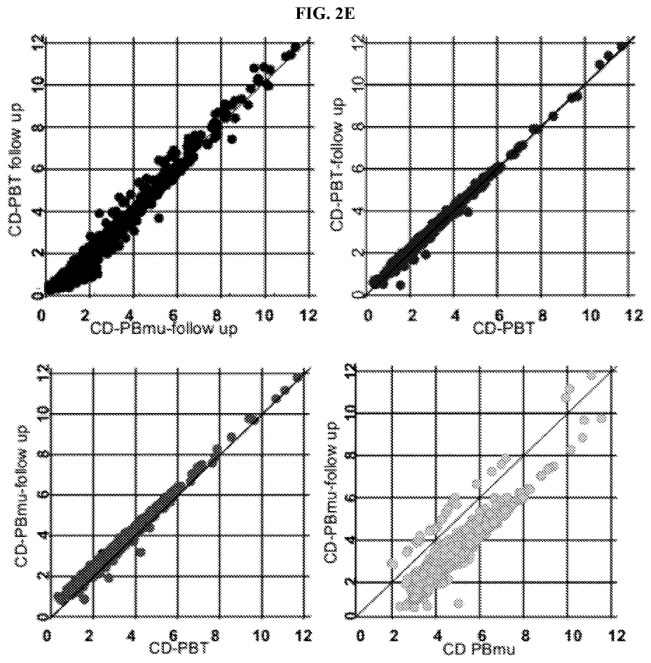

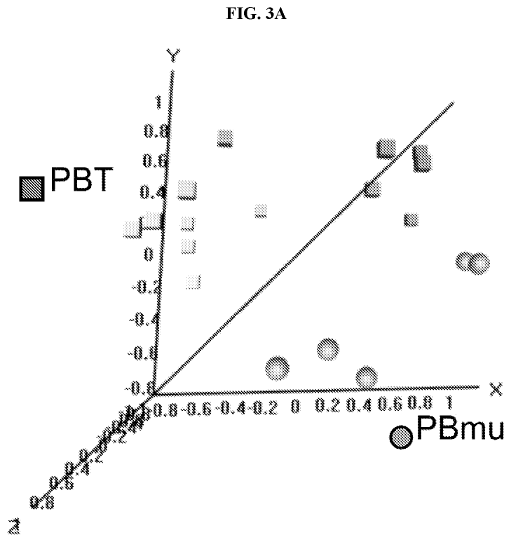

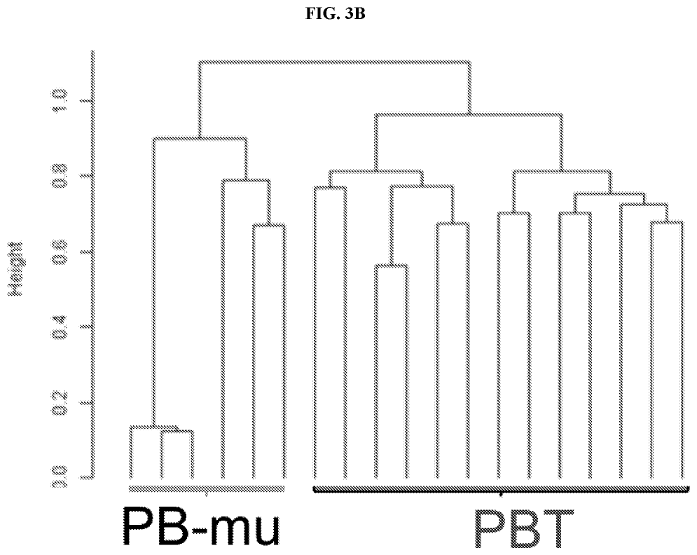

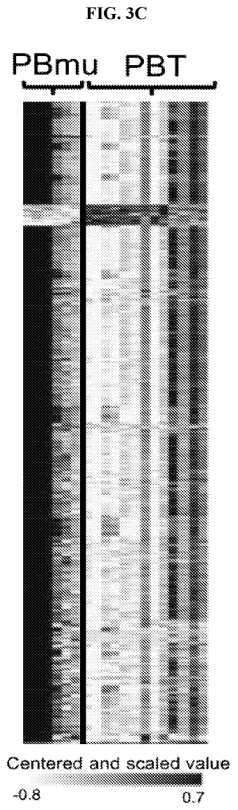

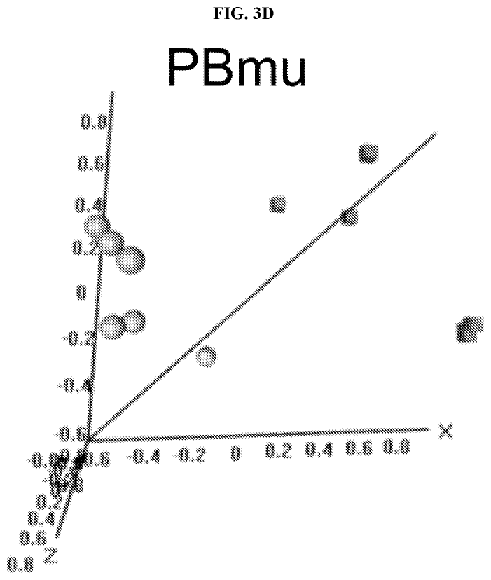

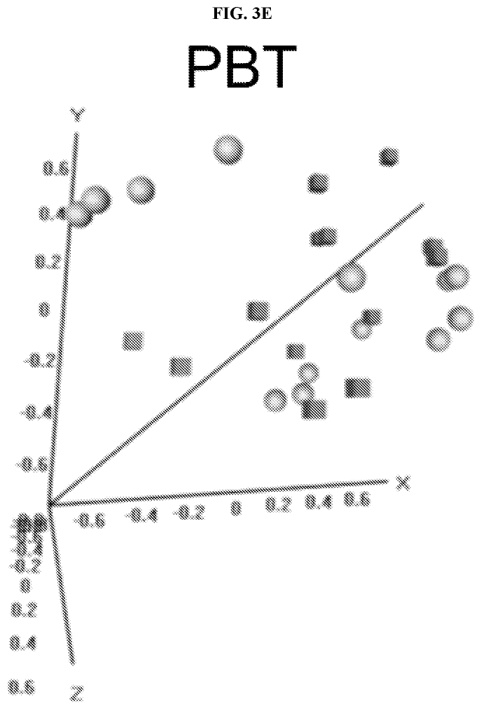

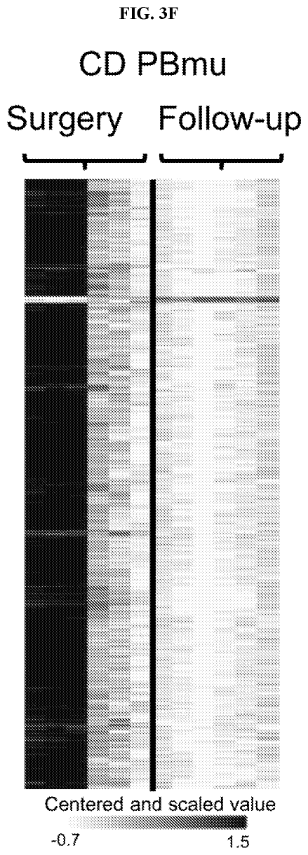

Longitudinal samples were collected from 30 CD patient 3-13 months post-surgery to assess the stability of the transcriptomic profiles. In patients classified as CD-PBmu, there was a significant alteration in gene expression following surgery (877 genes, p<0.001, FDR<0.013). Noticeably, the differentially over-expressed predictive transcriptomic signature which had defined the CD-PBmu subtype at the time of surgery, was no longer present after surgery (FIGS. 2A-B). Likewise, there was a downregulation of pro-inflammatory cytokine, chemokine and adhesion molecule expression following surgery (FIG. 2C). As seen in FIGS. 2D-2E, following surgery gene expression of the CD-PBmu-subtype reverts to that observed for the CD-PBT and non-IBD subjects at time of surgery, demonstrating a high correlation in expression between CD-PBmu subtype samples following surgery and CD-PBT subtype pre- or post-surgery. A separate independent CD cohort assessing the attenuation of the CD-PBmu profile (n=19) following surgery validated these findings (FIGS. 3A-3F). As seen in the PCA and heatmap plots there is a clear distinction in expression between the CD-PBmu and CD-PBT subtypes at the time of surgery (FIGS. 3A-3C). Furthermore, the genes defining the CD-PBmu samples pre and post-surgery in the initial cohort were validated and demonstrated a post-surgery alteration in gene expression exclusively in the CD-PBmu subtype (PCA analysis and heat map analysis, FIG. 3D-3F). No post-surgery alteration in gene expression was detected in CD-PBT subtype.

The CD-PBmu Up-Regulated Transcriptomic Signature is Similar to that of Ileal Biopsy Samples from Treatment-Naive Pediatric Patients with Crohn's Disease

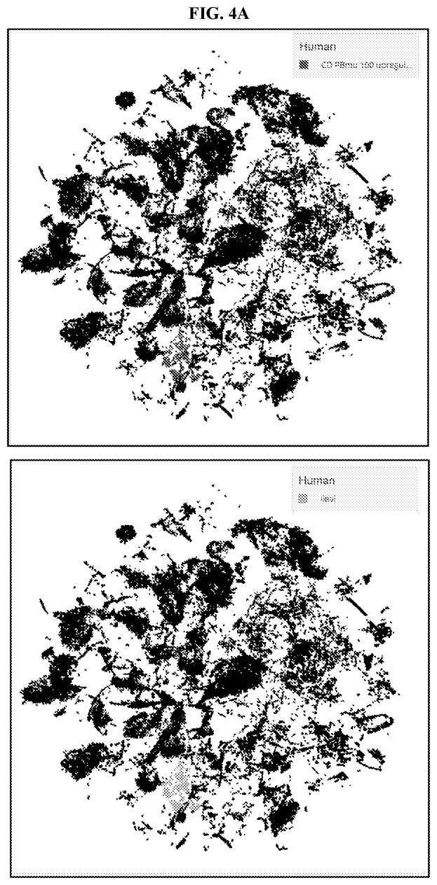

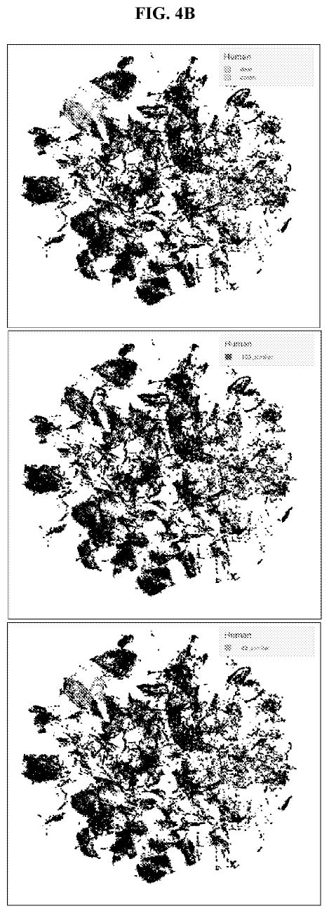

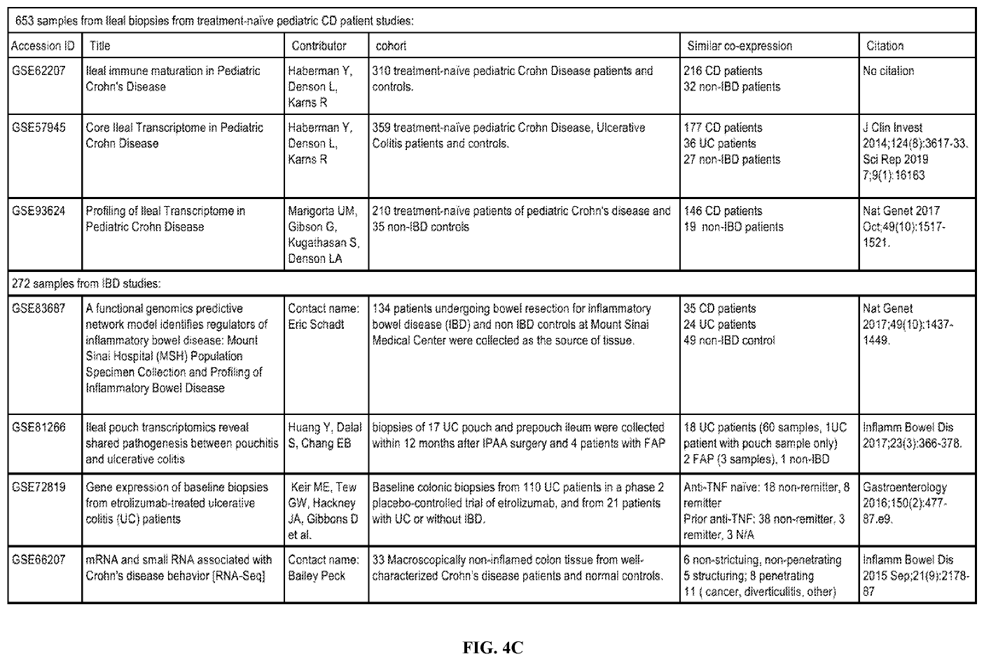

The ARCHS4 tool was utilized to compare the CD-PBmu transcriptomic signature (1944 transcripts) for similarity across multiple independent RNAseq studies (26,876 samples) for relationship discovery between gene expression and disease. A panel of 100 upregulated genes were used for analysis and samples identified by the ARCHS4 tool matching to the CD-PBmu input signature were downloaded. As seen in FIG. 4A, the CD-PBmu signature colocalized with ileal biopsy samples from inception studies of treatment naive pediatric Crohn's patients (n=751, 3 studies: GSE62207, GSE57945, GSE93624). The similarity of the CD-PBmu signature with ileal biopsy samples substantiates the mucosal origin of the circulating CD-PBmu peripheral T cells.

The ARCHS4 tool was further utilized to compare the CD-PBmu transcriptomic signature (1566 transcripts) for similarity across multiple independent RNAseq studies (26,876 samples) for relationship discovery between gene expression and disease. A panel of 193 upregulated genes (≥2 fold, t value 3.5-7) were used for analysis and samples identified by the ARCHS4 tool matching to the CD-PBmu input signature were downloaded. As seen in FIG. 4B and FIG. 4C, the CD-PBmu signature colocalized with ileal biopsy samples from inception studies of treatment naive pediatric Crohn's patients. The similarity of the CD-PBmu signature with ileal biopsy samples substantiates the mucosal origin of the circulating CD-PBmu peripheral T cells.

Findings were further validated in independent datasets with IBD patients (3 studies, n=338, GSE83687, GSE81266, GSE72819).

44-Gene Biomarker Classifier

Findings from the 1944 transcripts were refined into a 200 (Table 1A), 117 (Genes 1-117 of Table 1A), and then a 44-gene panel (Table 1A) to facilitate clinical application.

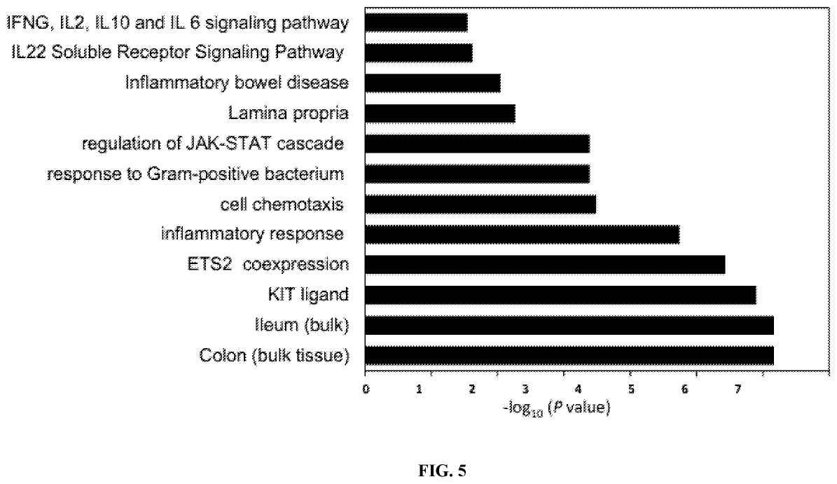

The 44-gene biomarker classifier was developed using both CD-PBmu vs CD-PBT differential expression and similarity with mucosal sample origin as a discriminator. Expression of the biomarker panel was assessed for correlation with the altered CD-PBmu T-cell subset composition. The 44-gene panel correlated with T cell subsets: NKT, CD4+ memory, CD4+ native, CD8+, CD4+, CD4+ Tcm, CD4+ Tem, CD8+ Tem, CD8+ Tcm, and CD8+ naive, as shown in FIGS. 7A-7B. All 44-genes displayed a significant positive correlation with the NKT cell enrichment score with the majority (42/44) associated with a p value of <1E-04 (FIG. 7A-7B). Conversely there was a negative correlation with >90% of the gene panel the CD4+ memory T cell enrichment score (34/44 with a p value of <0.001). The biomarker classifier likewise maintains the CD-PBmu vs CD-PBT classification with >80% accuracy and overlapped with TWAS signals predicted for associations with IBD (>60% of panel) (FIG. 7B). Pathway analysis of the 44-biomarker panel was validated in an IBD and mucosal association (FIG. 5). Moreover, the 44-gene panel was reflective of inflammatory and cytokine signaling pathways as well as regulation of the Jak/STAT signaling cascade.

The 44-gene biomarker panel includes A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), or phospholipase A2 group IIA (PLA2G2A).

In some cases, the 44-gene biomarker panel can be narrowed to a 27-gene biomarker panel with similar predictive capability as the 44-gene biomarker panel. The 27-gene biomarker panel, in some cases is ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, and UBD.

CD patients with severe disease can be stratified into 2 sub-populations based on transcriptomic profiling of their peripheral T-cells. A mucosal-like expression profile defined the CD-PBmu subtype which was associated with an altered composition of T-cell subsets, clinical disease severity markers and decreased pro-inflammatory gene expression following surgery. These findings hold potential to identify targets for patient-subtype specific therapeutic development. Moreover, the 44-gene biomarker panel confirmed the CD-PBmu gene signature in multiple independent pediatric CD datasets, suggesting this may provide a unique tool to improve accuracy in predicting clinical progression and facilitate treatment stratification early in the disease process.

42-Gene Biomarker Classifier

Findings from the 1566 transcripts were also refined into a 42-gene panel (Table 1B). The 42-gene biomarker classifier was developed using both CD-PBmu vs CD-PBT differential expression and a similarity with mucosal sample origin as a discriminator. A GSVA score generated for the 42-gene classifier maintained significant correlation with T cell subset enrichment scores (FIG. 4C). Expression of the biomarker panel was assessed for correlation with the altered CD-PBmu T-cell subset composition. All 42-genes displayed a significant positive correlation with the NKT cell enrichment scores with the majority (33/42) associated with a p value of <1E-06 (FIG. 7E). Conversely, there was a negative correlation (FIGS. 7E-7F, 1M-1N) of the gene panel expression with the CD4+/CD8+ T cell enrichment scores. The biomarker classifier likewise maintains the CD-PBmu vs CD-PBT classification (82% accuracy, Nonnegative matrix factorization clustering). Moreover, the 42-gene panel overlapped with TWAS signals predicted for associations with IBD as well as clinical association to perianal penetrating disease and ASCA sero-positivity (79% of panel) (FIG. 7E).

Table 13E provides a sample of unique CD-PBmu vs CD-PBT signature attributes.

TABLE 13E
Unique CD-PBmu vs CD-PBT signature attributes

Differential Gene Expression Using
Class Comparison Method

| | |
|---|---|
| Differential Gene Expression of | 1566 transcripts, p = 9.91E−04, |
| CD-PBmu vs CD-PBT | FDR < 0.002, fold > 2 |
| Enriched in pathways mediating | p = 9.9E−03 − 5.1E−07 |
| inflammatory response, leukocyte | |
| adhesion, migration and integrin | |
| binding | |

Identification of Potential Protein Kinase Signaling Pathways Regulating Expression of the CD-PBmu Transcriptomic Signature

Protein kinases are known mediators of chronic inflammation activating signaling pathways involved in cytokines/chemokines secretion, cellular activation, adhesion and migration. Protein kinases play a significant role in mediating pathogenesis of IBD as well. There is great interest in understanding how kinases are regulated by protein-protein interactions in order to identify additional therapeutic targets for drug intervention. A two-pronged approach was applied to discover candidate kinases likely to be involved in regulating CD-PBmu differential gene expression. Kinases were first identified in which there was a co-occurrence of increased gene expression prior to surgery and associated selective decrease postoperatively for the CD-PBmu vs. CD-PBT subtype (FIG. 7C). Twenty-five kinases displayed increased expression prior to surgery and selective post-surgical attenuation (˜2 fold) in CD-PBmu. In addition, the list of upstream kinases was expanded upon utilizing a kinase enrichment analysis (KEA3) tool. Genes with increased gene expression prior to surgery and associated selective decrease post-operatively for the CD-PBmu subtype were used for KEA3 analysis to infer as to which upstream kinases target these genes, as potential upstream regulators. The top 25 ranked kinases demonstrating significant association with CD-PBmu transcriptomic signature include cell cycle regulation (CDKs) and mTOR signaling kinase pathways (FIG. 7D, bars on the left). Moreover >70% of these kinases were validated using a separate analytical approach, X2k analysis, which combines transcription factor enrichment analysis, protein-protein interaction network expansion, with kinase enrichment analysis to predict upstream regulators (FIG. 7D, bars on the right). Disruption of many of these kinases have been targeted in clinical studies reinforcing the therapeutic implication associated with CD-PBmu differential gene expression.

TABLE 14
Selected Cytokines, Chemokines and Adhesion Molecules
Decreased in PB-mu Patient Subtype Follwoing Surgery

| Molecule | P value |
|---|---|
| IL10 | 1.7E−03 |
| IL11 | 4.0E−04 |
| IL15 | 1.8E−03 |
| IL18 | 1.9E−02 |
| IL22 | 8.5E−03 |
| IL6 | 1.0E−03 |
| IL12RB1 | 4.0E−04 |
| IL12RB2 | 1.1E−02 |
| IL17RD | 5.0E−04 |
| IL1R1 | 2.2E−03 |
| IL1RL1 | 7.9E−03 |
| IL31RA | 1.4E−03 |
| TNFRSF9 | 7.0E−04 |
| TNFSF14 | 3.3E−02 |
| TNFSF15 | 5.7E−03 |
| TNFAIP8L1 | 1.0E−03 |
| TNFAIP8L3 | 4.7E−03 |
| TNFRSF10A | 4.6E−02 |
| TNFRSF10B | 6.2E−03 |
| TNFRSF13B | 2.9E−02 |
| CCL11 | 1.1E−02 |
| CCL16 | 2.2E−03 |
| CCL21 | 2.7E−02 |
| CCL22 | 7.0E−04 |
| CCL28 | 5.5E−03 |
| CCL5 | 2.0E−04 |
| CCR6 | 7.6E−03 |
| CCR9 | 4.0E−03 |
| CXCL1 | 2.3E−02 |
| CXCL12 | 1.9E−02 |
| CXCL13 | 8.2E−03 |
| CXCL14 | 8.0E−04 |
| CXCL16 | 2.3E−02 |
| CXCL3 | 3.4E−02 |
| CXCL9 | 1.0E−04 |
| CLDN10 | 3.4E−02 |
| CLDN16 | 1.0E−03 |
| CLDN19 | 2.0E−04 |
| CLDN3 | 1.2E−03 |
| ICAM4 | 4.0E−03 |
| ITGAX | 2.2E−02 |

Discussion

Even with significant advances in biologic therapies, many CD patients experience persistent active disease, elevated rates of recurrence, and requirement for surgical intervention, with a significant burden of health care costs and reduced quality of life. There is not yet a reliable molecular diagnostic approach to predict lack of therapeutic response or postoperative recurrence. In this experiment, a CD patient population was studied with severe refractive disease to identify molecular pathways underlying clinical disease course. Characterized herein are circulating peripheral T cell transcriptomic signatures that sub-stratifies these patients into two distinct molecular subtypes termed CD-PBmu and CD-PBT. Patients exhibiting a CD-PBT transcriptomic signature clustered tightly with non-IBD subjects. Patients classified as CD-PBmu displayed a transcriptomic signature that drifted towards a more mucosal T cell profile which mirrored an alteration in inferred T subset composition and which correlated with a distinct subset of clinical features associated with complicated/aggressive disease. Moreover, it was only within the circulating peripheral T cells of CD-PBmu patients, that subsequent to surgical resection of the inflamed bowel tissue, there was a marked downregulation of pro-inflammatory and adhesion molecule expression. These findings provide evidence for classification of biologically distinct subtypes in Crohn's disease patients with severe medically refractory disease based upon circulating peripheral T cell transcriptomic signature.

The high clinical heterogeneity and genetic complexity of CD has revealed that the underlying biological pathways driving disease differs between patients. Genetic, molecular, immunologic, and microbiome studies provide evidence that this complexity is not spectral, but rather modal, with some success in identifying subgroups sharing combinations of these traits, including potentially targetable causal pathways. Thus, the development of early and targeted therapeutics requires biomarkers qualified in defining such subgroups. The significance of the CD-PBmu transcriptomic signature is twofold. It has the prognostic potential to identify, in a minimally invasive manner, a subset of CD patients likely to develop severe disease which might be averted through early initiation of individualized therapy. Secondly, the transcriptomic signature has potential to serve as a companion diagnostic that identifies and predicts patient response to a particular drug or therapeutic pathway.

The CD-PBmu transcriptomic signature is unique in that is was identified as a peripheral signature within a subset of CD patients who have failed therapeutic intervention. It is important to put these findings within the context of other studies. Mucosal gene expression in non-inflamed colon tissue from CD adults undergoing surgery, and to a lesser extent, treatment-naive pediatric CD patients was classified into a colon-like profile suggestive of rectal disease and an ileum-like profile associated with need for postoperative biological therapy. Expression of the proposed top ileal-like and colon-like gene signatures were analyzed in the data set. T cell expression of ileal- and colonic signature genes tended to be low, however nearly all genes were significantly elevated in T cells isolated from the mucosa compared to the periphery. A small number of the ileum-specific genes (7/20) were elevated in mucosal T cells isolated from CD patients compared to non-IBD subjects. No difference in gene expression in peripheral T cells was detected when comparing the CD patient group as a whole to non-IBD subjects. However, when patients were sub-stratified based on their CD-PBmu vs CD-PBT classification, CD-PBmu patients showed significantly higher expression of both the ileal and colonic signature genes compared to either CD-PBT or non-IBD subjects. No sub-type differential gene expression was seen in T cells isolated from the mucosal compartment.

The molecular classification presented here identifying two clinically relevant CD subtypes, is unique in that it provides evidence for heterogeneity in a patient population who clinically have all failed in therapeutic treatment escalation with a similar pre-op severity score and requires surgical resection. Independent validation of the presence of the CD-PBmu gene signature in a whole blood expression dataset isolated from CD patients who failed anti-TNF therapy, and the overlap association of the CD-PBmu gene biomarker panel with upregulated co-expression in an inception treatment-naive pediatric CD ileal biopsy cohort underscores the potential clinical application of these findings to facilitate patient stratification and more effective treatment prior to surgical resection.

The balance of T cell trafficking from the periphery into the gut and subsequent recycling of activated T cells back to the periphery is tightly regulated and is essential for maintaining immune gut homoeostasis. Uncontrolled chronic intestinal inflammation in Crohn's disease is characterized by infiltration of circulating activated proinflammatory T cells in the mucosa. CD4+ T-cell infiltration in intestinal tissue of IBD patients is a key feature of chronic intestinal inflammation with enhanced accumulation in active disease. An imbalance in the mucosal NKT cell population has likewise been reported in CD patients with severe disease. A number of studies have in fact further defined an imbalance in other mucosal T cells subsets including Treg and Tcm associated with disease activity. However, the prognostic utility of these findings is limited in that mucosal sampling requires invasive procedures and often the site of disease is difficult to access. More recent studies have demonstrated alterations in the expression of T and B cell activation markers using flow cytometry in circulating lymphocytes isolated from CD and UC patients during disease flare and in remission. An emerging body of evidence suggests an important role of ‘gut-tropic’ circulating lymphocytes. It is therefore of particular significance that a subset of CD patients is identified with a circulating blood transcriptomic signature associated with a mucosal-like expression profile. Expression of both CCR9 and CCR6 gut homing chemokine receptors are elevated in the peripheral blood of CD-PBmu versus CD-PBT patient subtype. The present study notes altered T subset gene signature in circulating T cells from CD patient with severe disease. While these findings are based upon imputed CD-PBmu cell subsets they provide a solid basis for future in depth studies to further evaluate alterations in T cell subsets directly by immunologic methods. It is of interest to note that the balance of the T cell composition ratio in matched paired samples between the periphery and mucosa is skewed in the CD-PBmu patient subtype with a more pronounced increase in the peripheral NKT signature and an associated pronounced decrease in the mucosal T cells compared to the CD-PBT subtype. Conversely, an inverse skewed balance between the periphery and mucosa was seen for the CD4+ memory T cell signature. These findings suggest that dysregulation of circulating intestinal-homing lymphocytes within the CD-PBmu subtype may underlie the molecular pathways mediating uncontrolled intestinal inflammation within this patient population.

Kinase dysregulation has been demonstrated as an underlying mechanism involved in the pathogenesis of IBD. Kinase inhibitor drug discovery is therefore of interest as a new therapeutic option. The CD-PBmu transcriptomic signature has potential to aid in guiding decisions as to which patients may benefit most from these targeted strategies. The kinase signaling pathways identified by both expression data as well as bioinformatic approaches identified enhanced activation of the MAP and AKT1 signaling pathways associated with CD-PBmu. Many of these identified kinases are intertwined and have been associated with IBD. AKT for example is involved in activation of the mTOR complex and GSK3β kinase is a downstream target of AKT. Activation of NF-KB occurs through the PI3K/AKT pathway and AKT is believed to have a role in attenuation of Tregs regulation of Th1/Th17 responses. Likewise, CSNK2A1, a subunit of the CK2 kinase, has been demonstrated to be a major regulator of the Treg-Th17 axis involved in Crohn's disease inflammation. CK2 interacts with JNKs and is essential for JAK-STAT activation. A number of therapeutic agents have been developed targeting members of these kinase pathways. In particular there has been an interest in the potential of mTOR and RIPK inhibitors for therapeutic intervention of IBD. It is interesting to note the association of FLT1 kinase with the CD-PBmu signature. FLT1 mRNA is increased in active UC and has been identified as a regulator of pulmonary, kidney and liver fibrosis and may serve as a potential new drug target for attenuating fibrosis in IBD.

This experiment addresses transcriptomic changes in peripheral T cells in CD patients prior and subsequent to surgery. Transcriptomic changes after surgery were detected selectively in CD patients classified with CD-PBmu subtype signature. Moreover, in contrast to serologic inflammatory markers that provide associative rather than causative information, attenuation of proinflammatory cytokine, chemokine and adhesion molecule expression after surgical resection likely provides insight into the causal pathways underlying inflammation in these patients. Recent accumulating and intriguing evidence suggest that early surgical intervention may in fact improve disease outcome in a select CD population with ileo-colonic disease. Considering that post-surgical alteration in gene expression was exclusive for the CD-PBmu subtype, the transcriptomic signature might provide insight into the biological underpinnings toward characterization of a patient population who might benefit from early surgical intervention.

Methods

Study Subjects

Human subjects were recruited through the MIRIAD IBD Biobank at the F. Widjaja Foundation Inflammatory Bowel and Immunobiology Research Institute at Cedars-Sinai Medical Center. Informed consent (approved by the Institutional Review Board at Cedars-Sinai Medical Center) was obtained from all participating subjects. Clinical information was obtained from CD patients prior to undergoing surgical resection after which patients were followed prospectively. Non-IBD subjects had no known history of IBD and underwent surgery for cancer (5/17, 29%), diverticulitis (4/17, 24%), familial adenomatous polyposis (2/17, 12%), polyps (3/17, 18%) and other (colonic inertia, trauma, or retained capsule, 3/17, 18%). All CD and non-IBD samples were collected from surgical resections performed by a single provider. A pre-operative severity score was calculated based on a modified disease severity weighted index. The attributes included fistula, perianal abscess, steroid use, biologics/immunologics use, stricture and disease extent. Intestinal resections were given a weighted score of 3 for subjects who has undergone previous resections and a score of 0 if this was their first resection. All laboratory procedures were performed by staff blinded to the patient clinical phenotype. Similarly, staff assessing patient phenotype were blinded to the results of all invitro assays. A pathological severity score was generated in which disease extent was calculated within surgical specimens in a blinded fashion to CD subtype classification. Criteria for diseased segments included extent of stricture, ulcer, fistula, and/or diseased mucosa. Subjects were stratified into 3 categories based on pathologic features and extent of disease: mild (<3 cm), moderate (3-5 cm), or severe (>5 cm, multiple fistula tracks, deep ulceration, and/or severe microscopic disease).

Isolation of Purified CD3+ Peripheral and Mucosal T Cells

Blood and intestinal specimens were obtained from CD patients undergoing surgical resection at Cedars-Sinai Medical Center, Los Angeles. PBMC were isolated by separation on Ficoll-Hypaque gradients. Lamina propria mononuclear cells (LPMC) were isolated from the resection samples. CD3+ T cells were isolated using CD3-immunomagnetic beads (Miltenyi Biotech, Auburn, Calif.), which allowed for isolation of at least 95% pure CD3+ T cells without T cell activation.

Gene Expression Assay for CD3+ T Cells and Whole Blood

Who RNA was extracted from CD3+ T cells and libraries for RNA-Seq were prepared with the Nugen human FFPE RNA-seq library system. The workflow consists of cDNA generation, fragmentation, end repair, adaptor ligation and PCR amplification. Different adaptors were used for multiplexing samples in one lane. Sequencing was performed on Illumina NextSeq 500 for a single read 75 run. All libraries were prepared using a single lot or reagents, equipment and processed by same technical staff. Samples were processed in two runs with technical and sample duplicates with negligible batch differences. Data quality check was done on Illumina SAV. Demultiplexing was performed with Illumina Bcl2fastq2 v 2.17 program. DESeq2 (v.1.18.1) was applied to produce normalized counts and the data were log 2-transformed. Clean, processed data along with respective meta-data was available in-house.

Transcriptomics of human whole blood from CD patients, refractory to anti-tumor necrosis factor-α treatment who participated in the CERTIFI study, was downloaded (Affymetrix HT HG-U133+PM Array Plate, GSE100833). The data processing methods were as previously described.

Statistical Analysis

RNAseq data analysis and data mining were performed using the BRB array tools (brb.nci.nih.gov/BRB-ArrayTools, version 4.6.1) and R-program (www.r-project.org). Class prediction analysis used Bayesian covariate predictor, diagonal linear discriminant analysis, k-nearest neighbor (using k=1 and 3), nearest centroid, support vector machines and non-negative matrix factorization multivariate classification methods, based upon a minimum p value of 0.001. A 0.632+ bootstrap cross-validation randomly re-sampling method was used to compute mis-classification rate. False Discovery Rate to control for multiple hypothesis testing was calculated by Benjamini and Hochberg method. Cluster analysis was performed using BRB array tools and Cluster 3.0 with Java Treeview. The xCELL algorithm and webtool was applied to the gene expression for T cell deconvolution of cell type specific abundance. Gene set variation analysis (GSVA) method was used to calculate single sample gene set enrichment. GSVA scores were generated as described using the 1566 PBmu DGE or 42 biomarker (Table 1B) gene set signatures. Tests for statistical significance were determined using JMP Statistical Software (Cary, N.C.). Data were assessed for normality by the Shapiro-Wilk test. If data were normal a 2-tailed, unpaired Student's t test was used. For non-normal data, Wilcoxon or KS test was used to calculate P values. A univariate model was fitted with CD subtypes for demographic and clinical data. There was no statistical significance between any demographic or clinical attributes when comparing CD-PBmu vs CD-PBT and multivariate analysis was not performed. Analysis for identifying the peripheral transcriptional signal alteration after surgery was performed by comparing paired sample for expression prior and post-surgery for individual patients.

Validation of CD-PBmu Signature

Whole blood gene expression from the CERTIFI study of Crohn's disease patients refractory to anti TNFalpha therapy was downloaded (accession number GSE100833). The range of CD activity index score from this study was 220-450 and median disease duration of 11 years. The expression data collected for validation was from CD patients at baseline, having failed on anti-TNF therapy and prior to drug (ustikinamab) treatment. Hierarchical clustering using the gene signature which had defined the CD-PBmu subtype was applied. Mean percent of correct cluster classification used Bayesian covariate predictor, diagonal linear discriminant analysis, k-nearest neighbor (using k=1 and 3), nearest centroid, support vector machines and non-negative matrix factorization and a bootstrap cross-validation prediction error of <0.01 based on 100 bootstrap samples. Cell type enrichment analysis was determined using the xCell webtool.

Pathway Analysis and Tissue Co-Expression Similarity

Pathway enrichment analysis of differentially expressed genes was determined using Qiagen Ingenuity Pathway Analysis and Ingenuity analysis match (IPA, Qiagen Redwood City; www.qiagen.com/ingenuity) and Enrichr (http://amp.pharm.mssm.edu/Enrichr/) or BRB array tools GO and KEGG pathway enrichment analysis. ARCHS4 (https://amp.pharm.mssm.edu/archs4) database tool was used to identify tissue signature similarity in co-expression.

A CD-PBmu gene signature of 116 differentially upregulated genes identified in validation data sets from time of surgery (p<0.001, ≥2 fold increase in expression) and post-surgery were used as input. GEO study identification numbers with significant co-expression were downloaded for tissue similarity analysis. Identification of TWAS, gene expression and genetic association and PheWAS pleiotropic disease and trait associations were determined using (http://twas-hub.org/genes/) and phenome-wide (https://phewascatalog.org/) tools.

CD-PBmu signature genes (restricted to HGNC approved symbols) with increased differential expression (>2) and a t value between 3.5 and 7 (n=193) were used as input for ARCHS4 analysis. GEO study identification numbers with significant co-expression were downloaded for tissue similarity analysis. The 42-gene biomarker classifier (Table 1B) was developed by sequential deletion of individual genes as input for ARCHS4 analysis and maintaining GEO mucosal signature for co-expression similarity. Identification of gene expression and genetic associations were determined using transcriptome-wide association (TWAS) (http://twas-hub.org/genes/) and pleiotropic disease and trait associations were determined using phenome-wide (PheWAS) (https://phewascatalog.org/) tools.

Microbial Antibody Responses

All blood samples were taken at the time of consent and enrollment. Sera were analyzed for expression of anti-glycan antibodies to Saccharomyces cerevisiae (ASCA), antibodies to the outer-membrane porin C of Escherichia coli (OmpC), a Pseudomonas fluorescens-associated sequence (12), antibodies against the flagellin CBir1 (anti-CBir1) and anti-neutrophil cytoplasmic antibodies (ANCA) in a blinded fashion by ELISA. Antibody levels were determined, and results expressed as ELISA units (EU/ml), which are relative to a Cedars-Sinai Laboratory standard, which is derived from a pool of patient sera with well-characterized disease found to have reactivity to this antigen. Quartile sum scores were generated and did not include ANCA positivity.

Kinase Signaling Pathways

Kinases over-expressed selectively in CD-PBmu at time of surgery were subjected to a Wilcoxon signed rank test to identify those kinases selectively decreased post-operatively for the CD-PBmu but not CD-PBT subtype. For inferring other potential upstream protein kinase signaling pathways regulating the CD-PBmu transcriptomic signature, the BRB class comparison analysis was used to identify genes overexpressed at time of surgery and decreased post-operatively (random variance model, nominal significance level set at 0.001). Protein kinase signaling pathways were identified using the top 100 class comparison genes identified as input in KEA3 (https://amp.pharm.mssm.edu/kea3/) which directly infers upstream kinases whose substrates are overrepresented in gene list and eXpression2Kinases (X2k) (https://amp.pharm.mssm.edu/X2K/) analysis which infers upstream regulatory networks from signatures of differentially expressed genes combining transcription factor enrichment analysis, protein-protein interaction network expansion, with kinase enrichment analysis.

### Example 2. Transcriptomic Profiling

Expression levels of each of genes 1-44 in Table 1A are determined in a CD patient using RNA sequencing. The patient's expression levels are compared to reference expression levels from subjects who have a PBT subtype. All of the 44-genes from the patient have expression levels at least 2-fold higher than the PBT reference. The patient is characterized as having a CD-PBmu subtype.

### Example 3. Identification of Therapeutic Agents

A library of compounds is screened for a subpopulation of compounds that modulate the activity and/or expression of one or more biomarkers of Table 15 or FIG. 7D, or of a biomolecule in a pathway of the one or more biomarkers of Table 15 or FIG. 7C. The subpopulation of compounds is screened for efficacy in an in vitro PBmu patient model to identify candidate therapeutic agents.

### Example 4. Monocyte Signature

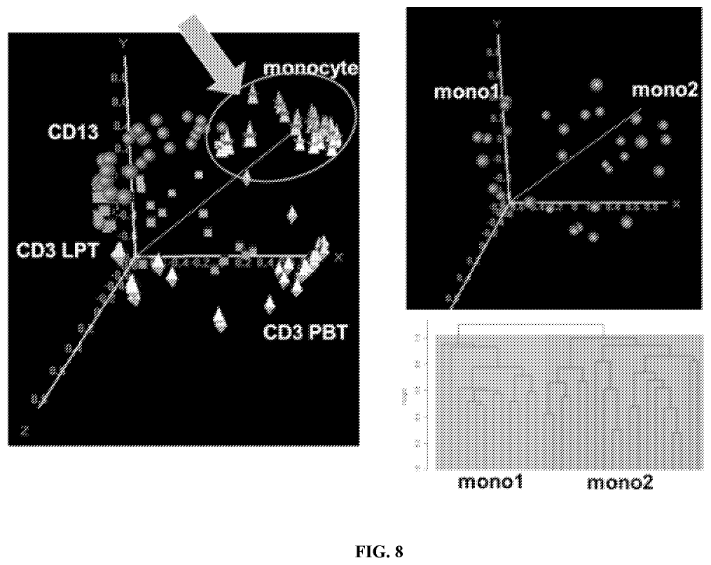

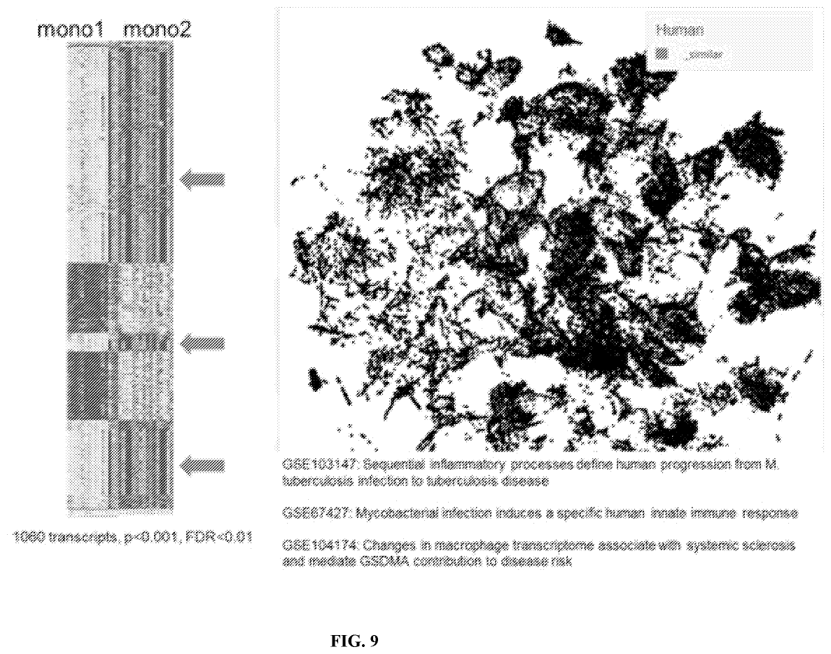

Peripheral and mucosal cells were obtained from untreated freshly isolated cells from 30 Crohn's disease (CD) subjects and 10 non-IBD subjects. RNA expression analysis was performed on peripheral CD3+ and monocyte cells, and mucosal CD3+ and CD13+ cells. Unsupervised clustering of CD monocytes revealed two signatures: monocyte 1 subtype (mono1) and monocyte 2 (mono2) subtype (FIG. 8). Differential gene expression in mono1 versus mono2 subtypes is shown in FIG. 9.

The CD mono2 subset was found to be associated with clinical and genetic parameters: ATG16L1 rs10210302 risk allele carriage (z score 2.2, p value 0.014), family history (z score 2.2, p value 0.014), IgG ASCA positive (z score 3, p value 0.0013), Serologic Quartile sum score (avg 11.4) (p value 0.049), failure on anti-TNF therapy (z score 1.8, p value 0.03), failure on 6-mercaptopurine/methotrexate (z score 3.4, p value 0.0004), and PBmu subjects (z score 1.4, p value 0.07).

### Example 5. Identifying Therapeutic Agents of Particular Relevance to PBmu CD Subtype

A two-tailed test was performed, which measured the statistical significance of an association of the differential gene expression of a target of interest in the PBmu patient subset. Table 15 provides a list of putative therapeutic targets, the differential expression of which, are statistically associated with the PBmu subtype.

TABLE 15
Therapeutic Targets for PBmu Subtype

| Gene | Pbmu | PBT | Prob > \|t\| |
|---|---|---|---|
| ADCY7 | 19.91897 | 24.43544 | 2.86E−03 |
| GPR65 | 32.85385 | 18.49456 | 4.62E−05 |
| GSDMB | 8.521538 | 5.792059 | 2.07E−04 |
| ICAM3 | 64.45026 | 84.52338 | 3.61E−06 |
| MAP4K4 | 24.32692 | 27.24235 | 4.35E−02 |
| PRKCQ | 23.15692 | 28.36426 | 2.42E−04 |
| PTGER4 | 23.70487 | 34.84235 | 7.49E−04 |
| RNASET2 | 60.94795 | 77.84529 | 6.13E−04 |
| TNFSF15 | 3.208718 | 1.245882 | 1.46E−03 |

The biomarker panels herein are associated with kinases provided in FIG. 6 and FIGS. 7C-7D. Without being bound by any particular theory, CD-PBmu patients would likely benefit from a targeted therapy to the kinases provided in FIG. 6 and/or FIGS. 7C-7D.

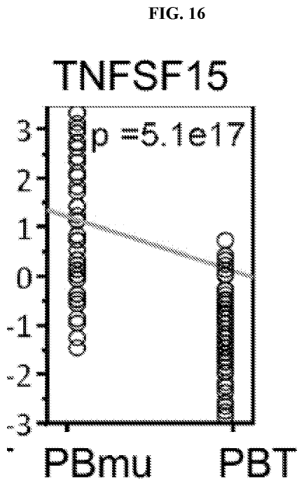

Expression of TNFSF15 (gene encoding TL1A) was measured in samples from patients classified as having the PBmu or PBT subtype. Expression of TNFSF15 was identified in PBmu patients, but not in patients having the PBT subtype (FIG. 16). Accordingly, provided herein are methods of treating patients having a PBmu subtype with an anti-TL1A antibody. Non-limiting exemplary antibodies include those described herein, such as those set forth in Table 18.

### Example 6. Monocyte Profiling

The expression level of one or more genes from Table 15 is determined in a CD patient using RNA sequencing. The patient's expression levels are compared to reference expression levels from subjects who have a mono1 or mono2 subtype. If the patient's expression levels are comparable to reference subjects having a mono2 subtype, the patient is characterized as having the mono2 CD subtype.

### Example 7. Treatment of Crohn's Disease Patient with PBmu Profile

The patient having the PBmu phenotype of Example 1 is treated with a candidate therapeutic agent of Example 3 or a therapeutic agent comprising a modulator of one or more of TL1A, ADCY7, GPR65, ICAM3, MAP4K4, PTGER4, RNASET2, TNFSF15; or an anti-TL1A antibody.

### Example 8. Treatment of Crohn's Disease Patient with Monocyte 2 Profile

The patient having the monocyte 2 subtype of Example 6 is treated with a candidate therapeutic agent targeting a kinase selected from: PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, and PKR. In one experiment, the therapeutic agent comprises one or more kinase modulators of Table 20B.

### Example 9. Pathways Enriched that Overlap with GWAS DEG in CD-PBmu Subtype

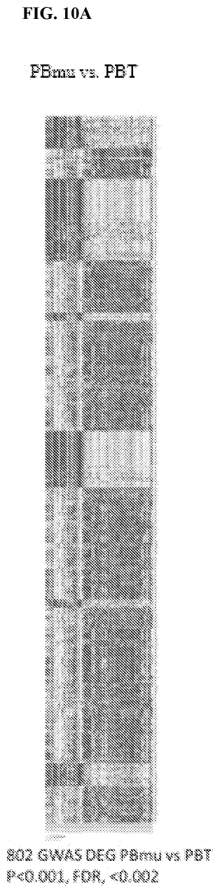

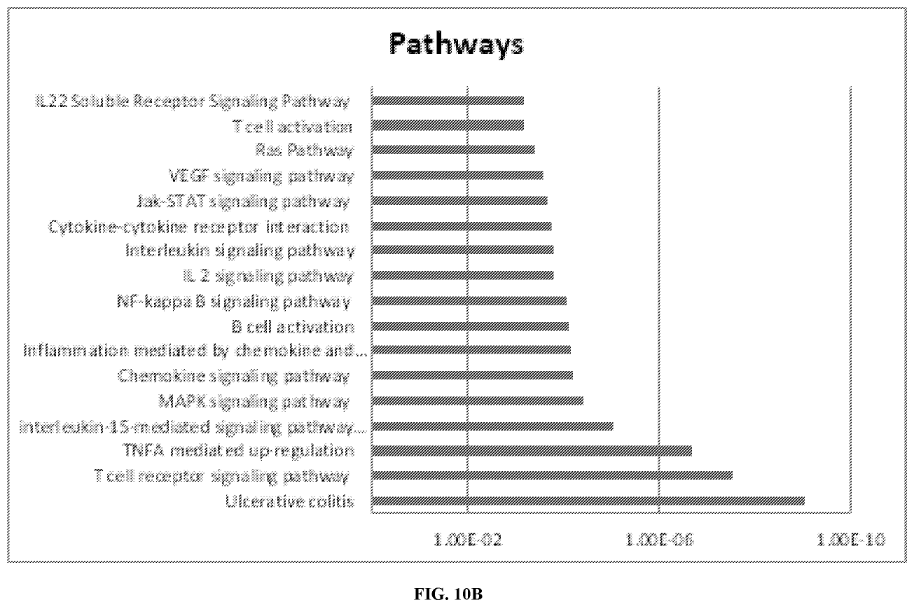

2616 genes potentially associated with IBD GWAS risk variant loci were identified. Of these genes, 1177 were not expressed in T cell data, 1429 were expressed in the T cell data, and 802 were differentially expressed between CD-PBmu and PBT subtypes (FIG. 10A). FIG. 10B shows pathways enriched that overlap with GWAS DEG CD-PBmu: IL22 soluble receptor signaling pathway, T cell activation, Ras pathway, VEGF signaling pathway, Jak-STAT signaling pathway, Cytokine-cytokine receptor interaction, interleukin signaling pathway, IL-2 signaling pathway, NF-kappa B signaling pathway, B cell activation, inflammation mediated by chemokine and cytokine signaling pathway, chemokine signaling pathway, MAPK signaling pathway, interleukin-15-mediated signaling pathway, TNF alpha mediated up-regulation, T cell receptor signaling pathway, and ulcerative colitis. In some examples, treatment of a patient having a CD-PBmu subtype comprises a molecule in one or more of the pathways shown in FIG. 10B.

### Example 10: miR-155 Expression is Relevant in CD-PBmu Subtype

CD3+ T cells were purified from paired blood and mucosal tissue from 101 CD patients and 17 non-IBD patients requiring surgery. Transcriptional profiles were generated by RNA-sequencing and T-cell subset composition was inferred by xCell.

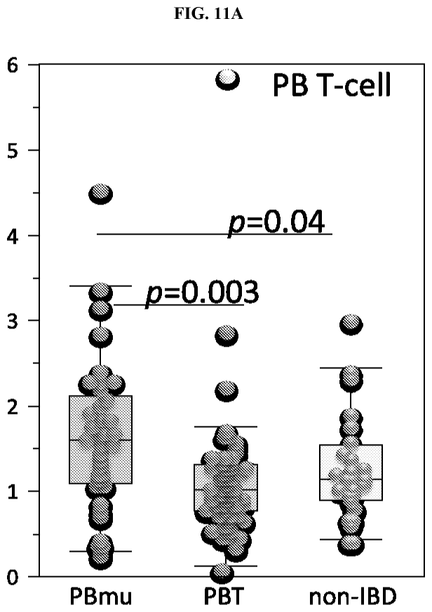

As seen on FIG. 11A, miR-155 expression was significantly increased in PB T-cells from patients with PB-mu subtype when compared to both non-IBD and PBT subtype samples. There was no significant change in expression levels in LP T-cells, as depicted in FIG. 11B.

### Example 11: miR-155 is Elevated in INFG Secreting CD4+ T-Cells

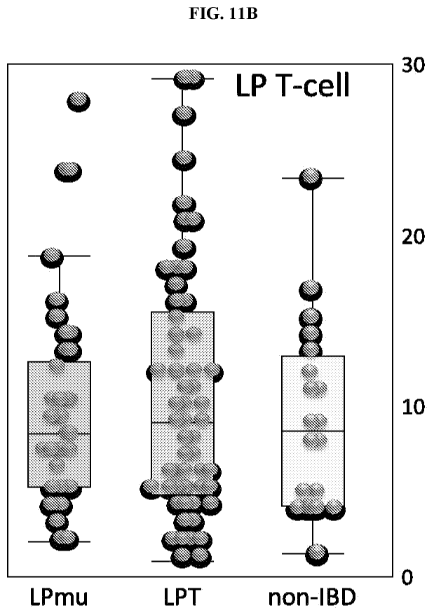

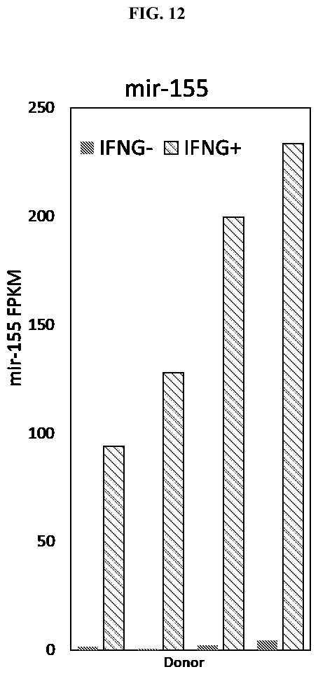

Transcriptional profiling of CD4+ T-cells was performed by RNA sequencing. T-cell subset composition was inferred by xCell. miR-155 expression was found to be elevated in INFG+ CD4+ T-cells, as compared to INFG− T-cells, as depicted in FIG. 12.

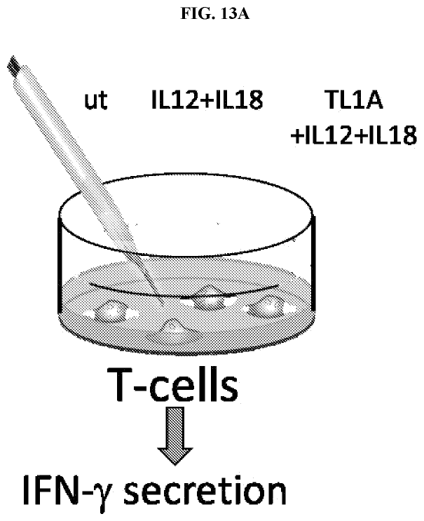

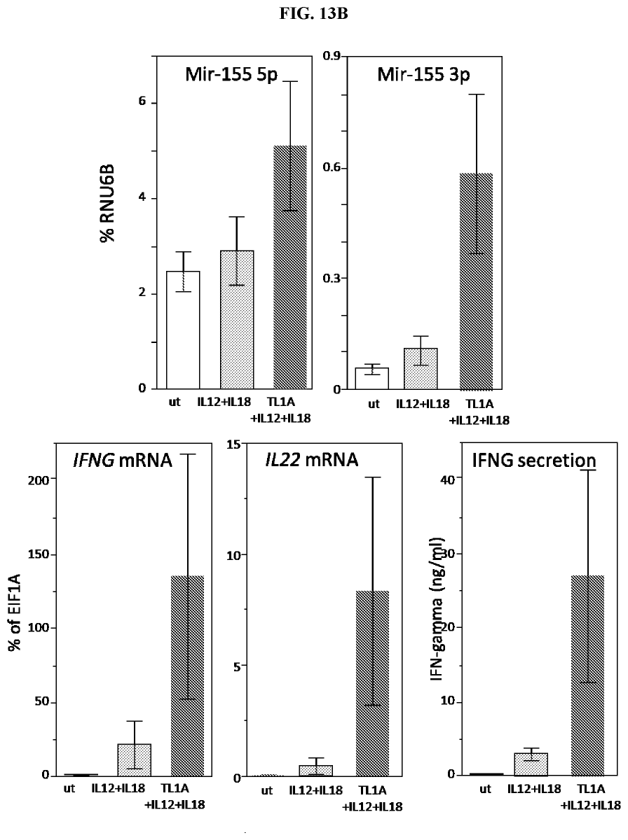

T-cells were divided into 3 treatment groups: cells treated with IL12+IL18, cells treated with TL1A+IL12+IL18, and untreated cells (ut), as depicted in FIG. 13A. Treatment with TL1A resulted in upregulation of both miR-155 5p, miR-155 3p when compared to cells that received no treatment or only IL12 and L18 treatment. Furthermore, treatment with TL1A also resulted in an increase in levels of both INFG mRNA and INFG secretion. IL22 mRNA was also increased in cells treated with TL1A.

### Example 12: miR-155 Mimic Enhances IFNG and IL22 Secretion and a miR-155 Inhibitor Suppresses INFG and ILL-22 Secretion

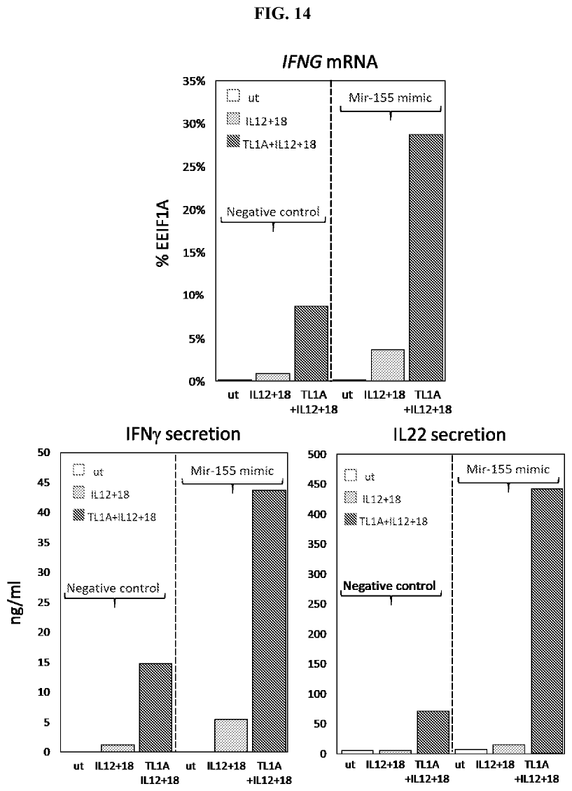

CD4+ T cells were rested overnight after isolation. Cells were then transfected with 150pmol (7.5 ul of 20 uM proper siRNA/mimic/inhibitor) for 10M cells in 250 ul Complete Media. Cells were rested overnight. Transfected cells were then divided into two groups and an interferon gamma blocking antibody was added to one group at 200 ng/ml final concentration. Both groups were further divided into 3 treatments of (untreated) UT, IL12+IL18 and TL1A+IL12+IL18. Cells were treated for 24h. Cells were collected and total RNA, and in some cases miRNA, were isolated. As depicted in FIG. 14, cells treated with mir-155 mimic showed an increase in levels of both IFNG mRNA and IFNG secretion when compared to the cells treated with a negative control. Furthermore, cells cultured with mir-155 mimic also showed an increase in IL22 secretion when compared to untreated controls. This increase was seen across all treatment groups.

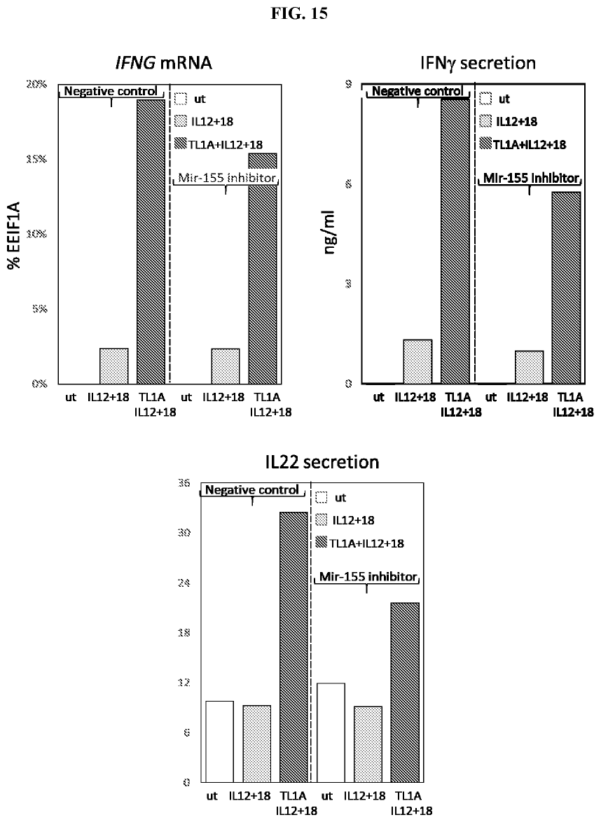

As depicted in FIG. 15, cells treated with mir-155 inhibitor showed a decrease in levels of both IFNG mRNA and IFNG secretion when compared to the cells treated with a negative control. Furthermore, cells cultured with mir-155 mimic also showed a decrease in IL22 secretion when compared to untreated controls. This decrease was seen across all treatment groups.

While preferred embodiments have been shown and described herein, it will be obvious to those skilled in the art that such embodiments are provided by way of example only. Numerous variations, changes, and substitutions will occur to those skilled in the art without departing from the scope of this application. Various alternatives to the embodiments described herein may be employed in practicing the scope of this application.

TABLE 16
Genes Associated with Transcriptomic Signature.

| | | fold | | | | | |
|---|---|---|---|---|---|---|---|
| | predi | Pbmu/ | | | | | |
| | ctor | post- | | | | | |
| | fold | surgery | | | | | |
| | Pbmu/ | follow | | Entrez | | | |
| Gene | PBT | up | Name | ID | Accession | UGCluster | Ensembl |
| AADACL2-AS1 | 6.09 | 6.44 | AADACL2 Antisense RNA 1 | 101928142 | | | ENSG00000242908 |
| AARS2 | 2.34 | 2.05 | alanyl-tRNA synthetase 2, | 57505 | NM_020745 | Hs.158381 | ENSG00000124608 |
| | | | mitochondrial | | | | |
| AASS | 3.55 | 2.96 | aminoadipate-semialdehyde | 10157 | NM_005763 | Hs.156738 | ENSG00000008311 |
| | | | synthase | | | | |
| ABCB5 | 4.08 | 2.97 | ATP-binding cassette, sub- | 340273 | NM_001163941 | Hs.404102 | ENSG00000004846 |
| | | | family B (MDR/TAP), | | | | |
| | | | member 5 | | | | |
| ABCC9 | 4.77 | 3.61 | ATP-binding cassette, sub- | 10060 | NM_005691 | Hs.732701 | ENSG00000069431 |
| | | | family C (CFTR/MRP), | | | | |
| | | | member 9 | | | | |
| ABHD11 | 2.6 | 2.24 | abhydrolase domain | 83451 | NM_001145363 | Hs.647045 | ENSG00000106077 |
| | | | containing 11 | | | | |
| ACADSB | 2.65 | 2.32 | acyl-CoA dehydrogenase, | 36 | NM_001609 | Hs.81934 | ENSG00000196177 |
| | | | short/branched chain | | | | |
| ACBD4 | 2.62 | 2.72 | acyl-CoA binding domain | 79777 | NM_001135704 | Hs.110298 | ENSG00000181513 |
| | | | containing 4 | | | | |
| ACBD7 | 4.58 | 3.42 | acyl-CoA binding domain | 414149 | NM_001039844 | Hs.644598 | ENSG00000176244 |
| | | | containing 7 | | | | |
| ADAMTS4 | 3.64 | 3.21 | ADAM metallopeptidase | 9507 | NM_005099 | Hs.211604 | ENSG00000158859 |
| | | | with thrombospondin type 1 | | | | |
| | | | motif, 4 | | | | |
| ADAT1 | 2.23 | 2 | adenosine deaminase, tRNA- | 23536 | NM_012091 | Hs.729312 | ENSG00000065457 |
| | | | specific 1 | | | | |
| ADRA1A | 3.91 | 3.36 | adrenoceptor alpha 1A | 148 | NM_000680 | Hs.709175 | ENSG00000120907 |
| AFMID | 3.83 | 3.03 | arylformamidase | 125061 | NM_001010982 | Hs.558614 | ENSG00000183077 |
| AICDA | 4.43 | 3.46 | activation-induced cytidine | 57379 | NM_020661 | Hs.149342 | ENSG00000111732 |
| | | | deaminase | | | | |
| AIPL1 | 4.1 | 3.6 | aryl hydrocarbon receptor | 23746 | NM_001033054 | Hs.279887 | ENSG00000129221 |
| | | | interacting protein-like 1 | | | | |
| AK3 | 2.27 | 1.98 | adenylate kinase 3 | 50808 | NM_001199852 | Hs.732022 | ENSG00000147853 |
| AKAP5 | 3.11 | 2.76 | A kinase (PRKA) anchor | 9495 | NM_004857 | Hs.656683 | ENSG00000179841 |
| | | | protein 5 | | | | |
| AKIP1 | 3.7 | 2.86 | A kinase (PRKA) interacting | 56672 | NM_001206645 | Hs.131180 | ENSG00000166452 |
| | | | protein 1 | | | | |
| ALDH6AJ | 3.37 | 2.79 | aldehyde dehydrogenase 6 | 4329 | NM_001278593 | Hs.293970 | ENSG00000119711 |
| | | | family, member A1 | | | | |
| ALG1 | 2.74 | 2.19 | ALG1, | 56052 | NM_019109 | Hs.592086 | ENSG00000033011 |
| | | | chitobiosyldiphosphodolichol | | | | |
| | | | beta-mannosyltransferase | | | | |
| ALG1L | 3.44 | 3.36 | ALG1, | 200810 | NM_001015050 | Hs.591299 | ENSG00000189366 |
| | | | chitobiosyldiphosphodolichol | | | | |
| | | | beta-mannosyltransferase- | | | | |
| | | | like | | | | |
| ALG1L9P | 3.65 | 3.19 | asparagine-linked | 285407 | NR_073386 | Hs.546711 | ENSG00000248671 |
| | | | glycosylation 1-like 9, | | | | |
| | | | pseudogene | | | | |
| ANKLE1 | 4.38 | 3.25 | ankyrin repeat and LEM | 126549 | NM_001278443 | Hs.721610 | ENSG00000160117 |
| | | | domain containing 1 | | | | |
| ANKRD20A9P | 4.63 | 3.49 | ankyrin repeat domain 20 | 284232 | NR_027995 | Hs.679496 | |
| | | | family, member A9, | | | | |
| | | | pseudogene | | | | |
| ANP32A-IT1 | 3.07 | 2.55 | ANP32A intronic transcript 1 | 80035 | NM_001040150 | Hs.662150 | |
| AP1S3 | 3.85 | 3.15 | adaptor-related protein | 130340 | NM_001039569 | Hs.632555 | ENSG00000152056 |
| | | | complex 1, sigma 3 subunit | | | | |
| AP4B1-AS1 | 3.41 | 2.86 | AP4B1 antisense RNA 1 | 100287 722 | NR_037864 | Hs.664669 | ENSG00000226167 |
| AP4S1 | 2.79 | 2.43 | adaptor-related protein | 11154 | NM_001128126 | Hs.293411 | ENSG00000100478 |
| | | | complex 4, sigma 1 subunit | | | | |
| APOBEC3A | 4.49 | 3.41 | apolipoprotein B mRNA | 200315 | NM_001270406 | Hs.226307 | ENSG00000128383 |
| | | | editing enzyme, catalytic | | | | |
| | | | polypeptide-like 3A | | | | |
| APOBEC3B-AS1 | 4.84 | 3.26 | APOBEC3B antisense RNA 1 | 100874 530 | NR_104187 | Hs.626951 | ENSG00000249310 |
| APOL1 | 2.69 | 2.24 | apolipoprotein L, 1 | 8542 | NM_001136540 | Hs.114309 | ENSG00000100312 |
| APOL4 | 4.11 | 3.23 | apolipoprotein L, 4 | 80832 | NM_030643 | Hs.115099 | ENSG00000100336 |
| AQP6 | 4.2 | 3.48 | aquaporin 6, kidney specific | 363 | NM_001652 | Hs.54505 | ENSG00000086159 |
| ARGFX | 3.85 | 2.97 | arginine-fifty homeobox | 503582 | NM_001012659 | Hs.224976 | ENSG00000186103 |
| ARHGEF26-AS1 | 4.74 | 3.55 | ARHGEF26 antisense RNA 1 | 100507 524 | NR_037901 | Hs.370221 | ENSG00000243069 |
| ARIH2OS | 2.44 | 2.17 | ariadne homolog 2 opposite | 646450 | NM_001123040 | Hs.720727 | ENSG00000221883 |
| | | | strand | | | | |
| ARRDC3-AS1 | 3.78 | 2.91 | ARRDC3 antisense RNA 1 | 100129 716 | NR_02743. | Hs.116364 | ENSG00000281357 |
| ARSA | 2.71 | 2.3 | arylsulfatase A | 410 | NM_000487 | Hs.88251 | ENSG00000100299 |
| ASTN2 | 4.05 | 3.07 | astrotactin 2 | 23245 | NM_001184734 | Hs.601562 | ENSG00000148219 |
| ATAD3C | 3.66 | 3.08 | ATPase family, AAA | 219293 | NM_001039211 | Hs.724767 | ENSG00000215915 |
| | | | domain containing 3C | | | | |
| ATCAY | 4.24 | 3.42 | ataxia, cerebellar, Cayman | 85300 | NM_033064 | Hs.418055 | ENSG00000167654 |
| | | | type | | | | |
| B3GNT6 | 4.52 | 3.67 | UDP-GlcNAc:betaGal beta- | 192134 | NM_138706 | Hs.352622 | ENSG00000198488 |
| | | | 1,3-N- | | | | |
| | | | acetylglucosaminyltransferase 6 | | | | |
| BAIAP2-AS1 | 3.08 | 2.86 | BAIAP2 antisense RNA 1 | 440465 | NM_001004336 | Hs.448889 | ENSG00000226137 |
| | | | (head to head) | | | | |
| BBS5 | 4.12 | 3.56 | Bardet-Biedl syndrome 5 | 129880 | NM_15238 | Hs.233398 | ENSG00000163093 |
| BCDIN3D | 2.27 | 1.92 | BCDIN3 domain containing | 144233 | NM_181708 | Hs.142736 | ENSG00000186666 |
| BHMT2 | 3.9 | 3.18 | betaine--homocysteine S- | 23743 | NM_001178005 | Hs.114172 | ENSG00000132840 |
| | | | methyltransferase 2 | | | | |
| BIN3-IT1 | 2.99 | 2.57 | BIN3 intronic transcript 1 | 80094 | NM_025026 | Hs.675917 | |
| BMP7 | 4.73 | 3.55 | bone morphogenetic protein 7 | 655 | NM_001719 | Hs.473163 | ENSG00000101144 |
| BMS1P4 | 2.39 | 2.3 | BMS1 ribosome biogenesis | 729096 | NR_026592 | Hs.709171 | ENSG00000271816 |
| | | | factor pseudogene 4 | | | | |
| BMS1P5 | 2.6 | 2.44 | BMS1 ribosome biogenesis | 399761 | NM_001040053 | Hs.711898 | ENSG00000204177 |
| | | | factor pseudogene 5 | | | | |
| BMS1P6 | 2.03 | 2.31 | BMS1 ribosome biogenesis | 642826 | NR_024495 | Hs.463017 | |
| | | | factor pseudogene 6 | | | | |
| BNIPL | 4.17 | 3.13 | BCL2/adenovirus E1B 19 kD | 149428 | NM_001159642 | Hs.591473 | ENSG00000163141 |
| | | | interacting protein like | | | | |
| BPNT1 | 2.68 | 2.19 | 3′(2′),5′-bisphosphate | 10380 | NM_001286149 | Hs.406134 | ENSG00000162813 |
| | | | nucleotidase 1 | | | | |
| BREA2 | 3.07 | 2.25 | breast cancer estrogen- | 286076 | NM_001024610 | Hs.178095 | |
| | | | induced apoptosis 2 | | | | |
| BRIP1 | 4.26 | 3.38 | BRCA1 interacting protein | 83990 | NM_032043 | Hs.128903 | ENSG00000136492 |
| | | | C-terminal helicase 1 | | | | |
| BSN-AS2 | 4.42 | 3.38 | BSN antisense RNA 2 (head | 100132 677 | NR_038866 | Hs.435651 | ENSG00000226913 |
| | | | to head) | | | | |
| C12orf65 | 3.15 | 2.61 | chromosome 12 open | 91574 | NM_001143905 | Hs.319128 | ENSG00000130921 |
| | | | reading frame 65 | | | | |
| C12orf77 | 4.03 | 3.21 | chromosome 12 open | 196415 | NM_001101339 | Hs.434453 | |
| | | | reading frame 77 | | | | |
| C14orf105 | 3.8 | 3.53 | chromosome 14 open | 55195 | NM_001283056 | Hs.659706 | ENSG00000100557 |
| | | | reading frame 105 | | | | |
| C14orf178 | 3.54 | 3.36 | chromosome 14 open | 283579 | NM_001173978 | Hs.375834 | ENSG00000197734 |
| | | | reading frame 178 | | | | |
| C17orf75 | 3.56 | 2.87 | chromosome 17 open | 64149 | NM_022344 | Hs.655257 | ENSG00000108656 |
| | | | reading frame 75 | | | | |
| C19orf35 | 5.46 | 5.13 | chromosome 19 open | 374872 | NM_198532 | Hs.511803 | ENSG00000188305 |
| | | | reading frame 35 | | | | |
| C1orf174 | 2.88 | 2.45 | chromosome 1 open | 339448 | NM_207356 | Hs.103939 | ENSG00000198912 |
| | | | reading frame 174 | | | | |
| C1orf210 | 4.09 | 3.15 | chromosome 1 open | 149466 | NM_001164829 | Hs.158963 | ENSG00000253313 |
| | | | reading frame 210 | | | | |
| C1orf229 | 5.51 | 3.73 | chromosome 1 open | 388759 | NM_207401 | Hs.456511 | |
| | | | reading frame 229 | | | | |
| C1QTNF6 | 2.67 | 2.39 | C1q and tumor necrosis | 114904 | NM_031910 | Hs.22011 | ENSG00000133466 |
| | | | factor related protein 6 | | | | |
| C21orf62 | 4.33 | 3.47 | chromosome 21 open | 56245 | NM_001162495 | Hs.517235 | ENSG00000205929 |
| | | | reading frame 62 | | | | |
| C2orf91 | 5.18 | 4.02 | chromosome 2 open | 400950 | NM_001242815 | Hs.738713 | ENSG00000205086 |
| | | | reading frame 91 | | | | |
| C3orf33 | 3 | 2.97 | chromosome 3 open | 285315 | NM_001308229 | Hs.350846 | ENSG00000174928 |
| | | | reading frame 33 | | | | |
| C4orf19 | 3.63 | 3.26 | chromosome 4 open | 55286 | NM_001104629 | Hs.107527 | ENSG00000154274 |
| | | | readingframe 19 | | | | |
| C4orf26 | 4.14 | 3.6 | chromosome 4 open | 152816 | NM_001206981 | Hs.24510 | ENSG00000174792 |
| | | | reading frame 26 | | | | |
| C6orf25 | 2.81 | 2.48 | chromosome 6 open | 80739 | NM_025260 | Hs.247879 | ENSG00000204420 |
| | | | reading frame 25 | | | | |
| C7orf55 | 3.94 | 3.41 | chromosome 7 open | 154791 | NM_197964 | Hs.718441 | ENSG00000164898 |
| | | | reading frame 55 | | | | |
| C8orf44 | 3.44 | 3.16 | chromosome 8 open | 56260 | NM_019607 | Hs.661238 | ENSG00000213865 |
| | | | reading frame 44 | | | | |
| C9orf3 | 2.69 | 2.13 | chromosome 9 open | 84909 | NM_001193329 | Hs.434253 | ENSG00000148120 |
| | | | reading frame 3 | | | | |
| CABP4 | 3.24 | 3.29 | calcium binding protein 4 | 57010 | NM_001300895 | Hs.143036 | ENSG00000175544 |
| CASC9 | 4.66 | 3.98 | cancer susceptibility | 101805 492 | NR_103848 | Hs.571424 | |
| | | | candidate 9 (non-protein | | | | |
| | | | coding) | | | | |
| CC2D2A | 3.76 | 3.39 | coiled-coil and C2 domain | 57545 | NM_001080522 | Hs.590928 | ENSG00000048342 |
| | | | containing 2A | | | | |
| CCDC122 | 3.01 | 2.62 | coiled-coil domain | 160857 | NM_144974 | Hs.170849 | ENSG00000151773 |
| | | | containing 122 | | | | |
| CCDC142 | 3.75 | 2.99 | coiled-coil domain | 84865 | NM_032779 | Hs.430199 | ENSG00000135637 |
| | | | containing 142 | | | | |
| CCDC148 | 5.74 | 3.89 | coiled-coil domain | 130940 | NM_001171637 | Hs.668597 | ENSG00000153237 |
| | | | containing 148 | | | | |
| CCDC30 | 3.52 | 2.99 | coiled-coil domain | 728621 | NM_001080850 | Hs.729640 | ENSG00000186409 |
| | | | containing 30 | | | | |
| CCL22 | 3.73 | 3.09 | chemokine (C-C motif) | 6367 | NM_002990 | Hs.534347 | ENSG00000102962 |
| | | | ligand 22 | | | | |
| CCL5 | 2.14 | 1.84 | chemokine (C-C motif) | 6352 | NM_001278736 | Hs.514821 | ENSG00000271503 |
| | | | ligand 5 | | | | |
| CD24 | 4.3 | 2.67 | CD24 molecule | 100133 941 | NM_001291737 | Hs.644105 | ENSG00000272398 |
| CD300LG | 4.84 | 3.95 | CD300 molecule-like family | 146894 | NM_001168322 | Hs.147313 | ENSG00000161649 |
| | | | member g | | | | |
| CD3EAP | 3.67 | 2.87 | CD3e molecule, epsilon | 10849 | NM_001297590 | Hs.710495 | ENSG00000117877 |
| | | | associated protein | | | | |
| CD82 | 3.21 | 2.56 | CD82 molecule | 3732 | NM_001024844 | Hs.527778 | ENSG00000085117 |
| CDH23 | 3.32 | 2.79 | cadherin-related 23 | 64072 | NM_001171930 | Hs.656032 | ENSG00000107736 |
| CDKN2B-AS1 | 3.49 | 2.95 | CDKN2B antisense RNA 1 | 100048 912 | NR_003529 | Hs.493614 | ENSG00000240498 |
| CEACAM22P | 4.41 | 3.57 | carcinoembryonic antigen- | 388550 | NR_027754 | Hs.446909 | ENSG00000230666 |
| | | | related cell adhesion | | | | |
| | | | molecule 22, pseudogene | | | | |
| CEACAM8 | 4.28 | 3.04 | carcinoembryonic antigen- | 1088 | NM_001816 | Hs.41 | ENSG00000124469 |
| | | | related cell adhesion | | | | |
| | | | molecule 8 | | | | |
| CENPBD1P1 | 2.78 | 2.54 | CENPB DNA-binding | 65996 | NM_023939 | Hs.541177 | ENSG00000213753 |
| | | | domains containing 1 | | | | |
| | | | pseudogene 1 | | | | |
| CENPN | 2.66 | 2.18 | centromere protein N | 55839 | NM_001100624 | Hs.726537 | ENSG00000166451 |
| CEP41 | 3.09 | 2.44 | centrosomal protein 41 kDa | 95681 | NM_001257158 | Hs.368315 | ENSG00000106477 |
| CES3 | 5.51 | 4.61 | carboxylesterase 3 | 23491 | NM_001185176 | Hs.268700 | ENSG00000172828 |
| CFLAR | 2.01 | 1.87 | CASP8 and FADD-like | 8837 | NM_001127183 | Hs.390736 | ENSG00000003402 |
| | | | apoptosis regulator | | | | |
| CHP1 | 2.48 | 2.17 | calcineurin-like EF-hand | 11261 | NM_007236 | Hs.406234 | ENSG00000187446 |
| | | | protein 1 | | | | |
| CHP2 | 3.66 | 2.96 | calcineurin-like EF-hand | 63928 | NM_022097 | Hs.178589 | ENSG00000166869 |
| | | | protein 2 | | | | |
| CHRM3 | 4.65 | 3.87 | cholinergic receptor, | 1131 | NM_000740 | Hs.7138 | ENSG00000133019 |
| | | | muscarinic 3 | | | | |
| CHRNB1 | 2.9 | 2.34 | cholinergic receptor, | 1140 | NM_000747 | Hs.330386 | ENSG00000170175 |
| | | | nicotinic, beta 1 (muscle) | | | | |
| CHST6 | 3.77 | 3.11 | carbohydrate (N- | 4166 | NM_021615 | Hs.655622 | ENSG00000183196 |
| | | | acetylglucosamine 6-O) | | | | |
| | | | sulfotransferase 6 | | | | |
| CHTA | 2.59 | 2.11 | class II, major | 4261 | NM_000246 | Hs.701991 | ENSG00000179583 |
| | | | histocompatibility complex, | | | | |
| | | | transactivator | | | | |
| CKMT2-AS1 | 2.71 | 2.28 | CKMT2 antisense RNA 1 | 100131 067 | NR_034121 | Hs.655855 | ENSG00000247572 |
| CMBL | 4.29 | 3.43 | carboxymethylenebutenolidase | 134147 | NM_138809 | Hs.192586 | ENSG00000164237 |
| | | | homolog (Pseudomonas) | | | | |
| COA1 | 2.15 | 1.89 | cytochrome c oxidase | 55744 | NM_018224 | Hs.654779 | ENSG00000106603 |
| | | | assembly factor 1 homolog | | | | |
| COA7 | 3.1 | 2.43 | cytochrome c oxidase | 65260 | NM_023077 | Hs.349905 | ENSG00000162377 |
| | | | assembly factor 7 (putative) | | | | |
| COMMD2 | 2.34 | 1.97 | COMM domain containing 2 | 51122 | NM_016094 | Hs.432729 | ENSG00000114744 |
| COX10-AS1 | 2.4 | 2.23 | COX10 antisense RNA 1 | 100874 058 | NR_049718 | Hs.720411 | ENSG00000236088 |
| COX18 | 2.38 | 2.1 | COX18 cytochrome c | 285521 | NM_001033760 | Hs.356697 | ENSG00000163626 |
| | | | oxidase assembly factor | | | | |
| COX6B2 | 4.89 | 4.27 | cytochrome c oxidase | 125965 | NM_144613 | Hs.550544 | ENSG00000160471 |
| | | | subunit VIb polypeptide 2 | | | | |
| | | | (testis) | | | | |
| CPB2-AS1 | 3.85 | 3.34 | CPB2 antisense RNA 1 | 100509 894 | NR_046226 | Hs.626139 | ENSG00000235903 |
| CPM | 3.42 | 2.83 | carboxypeptidase M | 1368 | NM_001005502 | Hs.654387 | ENSG00000135678 |
| CPPED1 | 2.76 | 2.26 | calcineurin-like | 55313 | NM_001099455 | Hs.460002 | ENSG00000103381 |
| | | | phosphoesterase domain | | | | |
| | | | containing 1 | | | | |
| CRHR1-IT1 | 2.19 | 2.35 | CRHR1 intronic transcript 1 | 147081 | NM_152466 | Hs.128813 | ENSG00000204650 |
| CRLF2 | 4.72 | 4.02 | cytokine receptor-like | 64109 | NM_001012288 | Hs.287729 | ENSG00000205755 |
| | | | factor 2 | | | | |
| CRX | 4.76 | 3.8 | cone-rod homeobox | 1406 | NM_000554 | Hs.617342 | ENSG00000105392 |
| CRYBB2P1 | 3.5 | 2.54 | crystallin, beta B2 | 1416 | NR_033733 | Hs.571835 | ENSG00000100058 |
| | | | pseudogene 1 | | | | |
| CRYM-AS1 | 4.41 | 3.55 | CRYM antisense RNA 1 | 400508 | NM_001101368 | Hs.578949 | |
| CSAD | 3.09 | 2.47 | cysteine sulfinic acid | 51380 | NM_001244705 | Hs.279815 | ENSG00000139631 |
| | | | decarboxylase | | | | |
| CSTF3-AS1 | 4.09 | 3.38 | CSTF3 antisense RNA 1 | 338739 | NR_034027 | Hs.423476 | ENSG00000247151 |
| | | | (head to head) | | | | |
| CTBP2 | 2.56 | 2.38 | C-terminal binding protein 2 | 1488 | NM_001083914 | Hs.501345 | ENSG00000175029 |
| CTCFL | 4.52 | 3.12 | CCCTC-binding factor (zinc | 140690 | NM_001269040 | Hs.131543 | ENSG00000124092 |
| | | | finger protein)-like | | | | |
| CXorf36 | 4.4 | 3.54 | chromosome X open | 79742 | NM_024689 | Hs.98321 | ENSG00000147113 |
| | | | reading frame 36 | | | | |
| CXorf56 | 3.27 | 2.74 | chromosome X open | 63932 | NM_001170569 | Hs.248572 | ENSG00000018610 |
| | | | reading frame 56 | | | | |
| CYB5D2 | 2.48 | 2.3 | cytochrome b5 domain | 124936 | NM_001254755 | Hs.513871 | ENSG00000167740 |
| | | | containing 2 | | | | |
| CYP20A1 | 2.1 | 1.91 | cytochrome P450, family 20, | 57404 | NM_020674 | Hs.446065 | ENSG00000119004 |
| | | | subfamily A, polypeptide 1 | | | | |
| CYP4V2 | 2.25 | 2.04 | cytochrome P450, family 4, | 285440 | NM_207352 | Hs.587231 | ENSG00000145476 |
| | | | subfamily V, polypeptide 2 | | | | |
| CYP51A1 | 2.45 | 2.32 | cytochrome P450, family 51, | 1595 | NM_000786 | Hs.417077 | ENSG00000001630 |
| | | | subfamily A, polypeptide 1 | | | | |
| DAND5 | 4.28 | 3.4 | DAN domain family member | 199699 | NM_152654 | Hs.331981 | ENSG00000179284 |
| | | | 5, BMP antagonist | | | | |
| DAPP1 | 2.61 | 1.93 | dual adaptor of | 27071 | NM_001306151 | Hs.436271 | ENSG00000070190 |
| | | | phosphotyrosine and 3- | | | | |
| | | | phosphoinositides | | | | |
| DCUN1D2 | 4.15 | 3.39 | DCN1, defective in cullin | 55208 | NM_001014283 | Hs.682987 | ENSG00000150401 |
| | | | neddylation 1, domain | | | | |
| | | | containing 2 | | | | |
| DDX51 | 2.06 | 2.08 | DEAD (Asp-Glu-Ala-Asp) | 317781 | NM_175066 | Hs.445168 | ENSG00000185163 |
| | | | (SEQ ID NO: 801) box | | | | |
| | | | polypeptide 51 | | | | |
| DESI1 | 2.29 | 2.1 | desumoylating isopeptidase 1 | 27351 | NM_015704 | Hs.570455 | ENSG00000100418 |
| DFFA | 2.4 | 2.14 | DNA fragmentation factor, | 1676 | NM_004401 | Hs.484782 | ENSG00000160049 |
| | | | 45 kDa, alpha polypeptide | | | | |
| DFFB | 2.59 | 2.21 | DNA fragmentation factor, | 1677 | NM_001004285 | Hs.133089 | ENSG00000169598 |
| | | | 40 kDa, beta polypeptide | | | | |
| | | | (caspase-activated DNase) | | | | |
| DHODH | 2.44 | 2.15 | dihydroorotate | 1723 | NM_001025193 | Hs.654427 | ENSG00000102967 |
| | | | dehydrogenase (quinone) | | | | |
| DLEU2 | 3.35 | 2.69 | deleted in lymphocytic | 8847 | NR_002612 | Hs.547964 | |
| | | | leukemia 2 (non-protein | | | | |
| | | | coding) | | | | |
| DLGAP1-AS2 | 4.82 | 3.67 | DLGAPI antisense RNA 2 | 84777 | NM_032691 | Hs.659053 | |
| DLK2 | 3.07 | 2.84 | delta-like 2 homolog | 65989 | NM_001286655 | Hs.337251 | ENSG00000171462 |
| | | | (Drosophila) | | | | |
| DMC1 | 4 | 3.5 | DNA meiotic recombinase 1 | 11144 | NM_001278208 | Hs.339396 | ENSG00000100206 |
| DNAH17-AS1 | 3.55 | 2.95 | DNAH17 antisense RNA 1 | 100996 295 | NR_102401 | Hs.615304 | |
| DNAJC22 | 4.1 | 3.66 | DnaJ (Hsp40) homolog, | 79962 | NM_001304944 | Hs.659300 | ENSG00000178401 |
| | | | subfamily C, member 22 | | | | |
| DNAJC27-AS1 | 3.67 | 3.24 | DNAJC27 antisense RNA 1 | 729723 | NR_034113 | Hs.436366 | ENSG00000224165 |
| DNAJC9-AS1 | 3.6 | 2.87 | DNAJC9 antisense RNA 1 | 414245 | NR_038373 | Hs.661857 | ENSG00000236756 |
| DNAL1 | 2.89 | 2.54 | dynein, axonemal, light | 83544 | NM_001201366 | Hs.271270 | ENSG00000119661 |
| | | | chain 1 | | | | |
| DNASE1 | 2.67 | 2.36 | deoxyribonuclease I | 1773 | NM_005223 | Hs.629638 | ENSG00000213918 |
| DNM1P46 | 2.57 | 2.24 | dynamin 1 pseudogene 46 | 196968 | NM_194295 | Hs.567763 | ENSG00000182397 |
| DPH3P1 | 3.77 | 3.43 | diphthamide biosynthesis 3 | 100132 911 | NM_080750 | | |
| | | | pseudogene 1 | | | | |
| DPY19L1P1 | 2.75 | 3 | DPY19L1 pseudogene 1 | 100129 460 | NR_036680 | Hs.633705 | |
| DPY19L2P2 | 3.48 | 3.02 | DPY19L2 pseudogene 2 | 349152 | NM_182634 | Hs.732579 | ENSG00000170629 |
| DSG2 | 4.49 | 3.32 | desmoglein 2 | 1829 | NM_001943 | Hs.412597 | ENSG00000046604 |
| DSG3 | 3.99 | 3.27 | desmoglein 3 | 1830 | NM_001944 | Hs.1925 | ENSG00000134757 |
| DTD2 | 2.58 | 2.22 | D-tyrosyl-tRNA deacylase 2 | 112487 | NM_080664 | Hs.116014 | ENSG00000129480 |
| | | | (putative) | | | | |
| DUXA | 4.59 | 3.88 | double homeobox A | 503835 | NM_001012729 | Hs.585857 | ENSG00000258873 |
| DYDC1 | 3.82 | 3.41 | DPY30 domain containing 1 | 143241 | NM_001269053 | Hs.407751 | ENSG00000170788 |
| DYNAP | 5.01 | 3.36 | dynactin associated protein | 284254 | NM_001307955 | Hs.376146 | ENSG00000178690 |
| ECT2 | 3.62 | 2.68 | epithelial cell transforming 2 | 1894 | NM_001258315 | Hs.518299 | ENSG00000114346 |
| EEF2K | 2.06 | 1.87 | eukaryotic elongation factor | 29904 | NM_013302 | Hs.498892 | ENSG00000103319 |
| | | | 2 kinase | | | | |
| EFCAB11 | 3.61 | 3.02 | EF-hand calcium binding | 90141 | NM_001284266 | Hs.123232 | ENSG00000140025 |
| | | | domain 11 | | | | |
| EGFEM1P | 4.33 | 3.45 | EGF-like and EMI domain | 93556 | NR_021485 | Hs.478158 | |
| | | | containing 1, pseudogene | | | | |
| EID2B | 2.85 | 2.44 | EP300 interacting inhibitor | 126272 | NM_152361 | Hs.135181 | ENSG00000176401 |
| | | | of differentiation 2B | | | | |
| ELMOD1 | 4.61 | 3.72 | ELMO/CED-12 domain | 55531 | NM_001130037 | Hs.495779 | ENSG00000110675 |
| | | | containing 1 | | | | |
| EMP2 | 3.97 | 3.43 | epithelial membrane protein 2 | 2013 | NM_001424 | Hs.531561 | ENSG00000213853 |
| EMX2OS | 4.29 | 3.68 | EMX2 opposite | 196047 | NR_002791 | Hs.312592 | ENSG00000229847 |
| | | | strand/antisense RNA | | | | |
| ENTPD1 | 3.56 | 2.8 | ectonucleoside triphosphate | 953 | NM_001098175 | Hs.576612 | ENSG00000138185 |
| | | | diphosphohydrolase 1 | | | | |
| ENTPD1-AS1 | 3.75 | 3.18 | ENTPD1 antisense RNA 1 | 728558 | NR_038444 | Hs.538374 | ENSG00000226688 |
| EP300-AS1 | 5.34 | 3.95 | EP300 antisense RNA 1 | 101927 279 | NR_110514 | Hs.517517 | |
| EPGN | 4.64 | 3.69 | epithelial mitogen | 255324 | NM_001013442 | Hs.401237 | ENSG00000182585 |
| EPHA10 | 4.57 | 3.81 | EPH receptor A10 | 284656 | NM_001004338 | Hs.129435 | ENSG00000183317 |
| EPPIN | 4.35 | 3.47 | epididymal peptidase | 57119 | NM_001302861 | Hs.121084 | ENSG00000101448 |
| | | | inhibitor | | | | |
| ERVK13-1 | 2.07 | 1.94 | endogenous retrovirus group | 100507 321 | NM_001012731 | Hs.406976 | |
| | | | K13, member 1 | | | | |
| ERVV-1 | 4.55 | 4.02 | endogenous retrovirus group | 147664 | NM_152473 | Hs.44329 | ENSG00000269526 |
| | | | V, member 1 | | | | |
| ESRG | 5.79 | 4.25 | embryonic stem cell related | 790952 | NR_027122 | Hs.720658 | ENSG00000265992 |
| | | | (non-protein coding) | | | | |
| EXD1 | 4.36 | 3.54 | exonuclease 3′-5′ domain | 161829 | NM_001286441 | Hs.307999 | ENSG00000178997 |
| | | | containing 1 | | | | |
| EXOC3L2 | 3.78 | 2.96 | exocyst complex component | 90332 | NM_138568 | Hs.337557 | ENSG00000130201 |
| | | | 3-like 2 | | | | |
| EXPH5 | 3.01 | 2.73 | exophilin 5 | 23086 | NM_001144763 | Hs.28540 | ENSG00000110723 |
| F5 | 2.55 | 1.87 | coagulation factor V | 2153 | NM_000130 | Hs.30054 | ENSG00000198734 |
| | | | (proaccelerin, labile factor) | | | | |
| FAIM | 3.04 | 2.51 | Fas apoptotic inhibitory | 55179 | NM_001033030 | Hs.173438 | ENSG00000158234 |
| | | | molecule | | | | |
| FAM106A | 4.01 | 3.81 | family with sequence | 80039 | NM_024974 | Hs.674403 | ENSG00000213077 |
| | | | similarity 106, member A | | | | |
| FAM114A1 | 4.04 | 2.87 | family with sequence | 92689 | NM_138389 | Hs.476517 | ENSG00000197712 |
| | | | similarity 114, member A1 | | | | |
| FAM122C | 3.02 | 2.67 | family with sequence | 159091 | NM_001170779 | Hs.269127 | ENSG00000156500 |
| | | | similarity 122C | | | | |
| FAM153C | 3.38 | 3.2 | family with sequence | 653316 | NM_001079527 | Hs.652193 | ENSG00000204677 |
| | | | similarity 153, member C, | | | | |
| | | | pseudogene | | | | |
| FAM231A | 2.11 | 2.64 | family with sequence | 729574 | NM_001282321 | | ENSG00000237847 |
| | | | similarity 231, member A | | | | |
| FAM71F2 | 3.98 | 3.49 | family with sequence | 346653 | NM_001012454 | Hs.445236 | ENSG00000205085 |
| | | | similarity 71, member F2 | | | | |
| FAM73A | 2.49 | 2.21 | family with sequence | 374986 | NM_001270384 | Hs.437755 | ENSG00000180488 |
| | | | similarity 73, member A | | | | |
| FAM74A3 | 4.26 | 3.66 | family with sequence | 728495 | NM_001098718 | Hs.723007 | |
| | | | similarity 74, member A3 | | | | |
| FAM83H-AS1 | 4.13 | 3.28 | FAM83H antisense RNA 1 | 100128 338 | NR_033849 | Hs.493171 | ENSG00000203499 |
| | | | (head to head) | | | | |
| FBLIM1 | 4.42 | 3.64 | filamin binding LIM protein 1 | 54751 | NM_001024215 | Hs.530101 | ENSG00000162458 |
| FBLN1 | 5.56 | 4.26 | fibulin 1 | 2192 | NM_001996 | Hs.24601 | ENSG00000077942 |
| FBXL18 | 3.27 | 2.48 | F-box and leucine-rich repeat | 80028 | NM_024963 | Hs.623974 | ENSG00000155034 |
| | | | protein 18 | | | | |
| FBXO17 | 4.71 | 3.73 | F-box protein 17 | 115290 | NM_024907 | Hs.531770 | ENSG00000269190 |
| FBXO27 | 4.21 | 3.63 | F-box protein 27 | 126433 | NM_178820 | Hs.187461 | ENSG00000161243 |
| FBXO45 | 2.56 | 2.08 | F-box protein 45 | 200933 | NM_001105573 | Hs.169815 | ENSG00000174013 |
| FBXO6 | 2.95 | 2.66 | F-box protein 6 | 26270 | NM_018438 | Hs.464419 | ENSG00000116663 |
| FCAR | 4.27 | 3.38 | Fc fragment of IgA receptor | 2204 | NM_002000 | Hs.65987 | ENSG00000275136 |
| FDPSP2 | 4.04 | 3.16 | farnesyl diphosphate | 619190 | NR_003262 | Hs.609978 | ENSG00000233980 |
| | | | synthase pseudogene 2 | | | | |
| FEZ1 | 3.19 | 3.63 | fasciculation and elongation | 9638 | NM_005103 | Hs.224008 | ENSG00000149557 |
| | | | protein zeta 1 (zygin I) | | | | |
| FGD5P1 | 4.12 | 3.22 | FYVE, RhoGEF and PH | 100132 526 | NR_036481 | Hs.637770 | ENSG00000275340 |
| | | | domain containing 5 | | | | |
| | | | pseudogene 1 | | | | |
| FGF5 | 3.97 | 3.16 | fibroblast growth factor 5 | 2250 | NM_001291812 | Hs.37055 | ENSG00000138675 |
| FGFR1OP | 2.9 | 2.36 | FGFR1 oncogene partner | 11116 | NM_001278690 | Hs.487175 | ENSG00000213066 |
| FILIP1 | 5.4 | 4 | filamin A interacting protein 1 | 27145 | NM_001289987 | Hs.696158 | ENSG00000118407 |
| FKBP14 | 3.47 | 2.87 | FK506 binding protein 14, | 55033 | NM_017946 | Hs.390838 | ENSG00000106080 |
| | | | 22 kDa | | | | |
| FLCN | 2.43 | 2.18 | folliculin | 201163 | NM_144606 | Hs.31652 | ENSG00000154803 |
| FLJ31104 | 3.77 | 3.06 | uncharacterized LOC441072 | 441072 | NR_102755 | Hs.482141 | ENSG00000227908 |
| FLJ31356 | 4.5 | 3.72 | uncharacterized protein | 403150 | NR_103831 | Hs.562970 | ENSG00000229951 |
| | | | FLJ31356 | | | | |
| FLJ31662 | 5.11 | 4.05 | uncharacterized LOC440594 | 440594 | NR_033966 | Hs.514123 | ENSG00000233907 |
| FLJ42102 | 4.48 | 3.47 | uncharacterized LOC399923 | 399923 | NM_001001680 | Hs.128191 | ENSG00000172900 |
| FRMD6-AS1 | 53.64 | 71.46 | FRMD6 antisense RNA 1 | 145438 | NR_037676 | Hs.645410 | ENSG00000273888 |
| FRRS1 | 5.18 | 4.33 | ferric-chelate reductase 1 | 391059 | NM_001013660 | Hs.454779 | ENSG00000156869 |
| FRY-AS1 | 4.5 | 3.46 | FRY antisense RNA 1 | 100507 099 | NR_103839 | Hs.536364 | |
| FTX | 2.53 | 2.33 | FTX transcript, XIST | 100302 692 | NR_028379 | Hs.349570 | ENSG00000230590 |
| | | | regulator (non-protein | | | | |
| | | | coding) | | | | |
| FUT1 | 4.07 | 3.23 | fucosyltransferase 1 | 2523 | NM_000148 | Hs.69747 | ENSG00000174951 |
| | | | (galactoside 2-alpha-L- | | | | |
| | | | fucosyltransferase, H blood | | | | |
| | | | group) | | | | |
| FUT2 | 4.29 | 3.4 | fucosyltransferase 2 (secretor | 2524 | NM_000511 | Hs.579928 | ENSG00000176920 |
| | | | status included) | | | | |
| FUT6 | 3.68 | 3.11 | fucosyltransferase 6 (alpha | 2528 | NM_000150 | Hs.631846 | ENSG00000156413 |
| | | | (1,3) fucosyltransferase) | | | | |
| GAL3ST4 | 2.8 | 2.17 | galactose-3-O- | 79690 | NM_024637 | Hs.44856 | ENSG00000197093 |
| | | | sulfotransferase 4 | | | | |
| GALNT15 | 3.4 | 3.03 | polypeptide N- | 117248 | NM_054110 | Hs.411308 | ENSG00000131386 |
| | | | acetylgalactosaminyltransfer | | | | |
| | | | ase 15 | | | | |
| GAS6-AS2 | 4.23 | 3.72 | GAS6 antisense RNA 2 | 100506 394 | NR_044993 | Hs.132168 | ENSG00000272695 |
| | | | (head to head) | | | | |
| GATAD1 | 2.19 | 2.09 | GATA zinc finger domain | 57798 | NM_021167 | Hs.21145 | ENSG00000157259 |
| | | | containing 1 | | | | |
| GDPD1 | 3.86 | 3.45 | glycerophosphodiester | 284161 | NM_001165993 | Hs.631744 | ENSG00000153982 |
| | | | phosphodiesterase domain | | | | |
| | | | containing 1 | | | | |
| GEMIN8 | 3.59 | 2.91 | gem (nuclear organelle) | 54960 | NM_001042479 | Hs.592237 | ENSG00000046647 |
| | | | associated protein 8 | | | | |
| GFOD2 | 3.21 | 2.57 | glucose-fructose | 81577 | NM_001243650 | Hs.307084 | ENSG00000141098 |
| | | | oxidoreductase domain | | | | |
| | | | containing 2 | | | | |
| GGT6 | 4.3 | 3.55 | gamma-glutamyltransferase 6 | 124975 | NM_001122890 | Hs.130749 | ENSG00000167741 |
| GGT8P | 4.96 | 3.97 | gamma-glutamyltransferase | 645367 | NR_003503 | Hs.650223 | |
| | | | 8 pseudogene | | | | |
| GK5 | 2.43 | 2.21 | glycerol kinase 5 (putative) | 256356 | NM_001039547 | Hs.135904 | ENSG00000175066 |
| GLIPR1L2 | 5.27 | 3.7 | GLI pathogenesis-related 1 | 144321 | NM_001270396 | Hs.406728 | ENSG00000180481 |
| | | | like 2 | | | | |
| GNB4 | 3.49 | 2.72 | guanine nucleotide binding | 59345 | NM_021629 | Hs.173030 | ENSG00000114450 |
| | | | protein (G protein), beta | | | | |
| | | | polypeptide 4 | | | | |
| GNE | 3.01 | 2.47 | glucosamine (UDP-N- | 10020 | NM_001128227 | Hs.5920 | ENSG00000159921 |
| | | | acetyl)-2-epimerase/N- | | | | |
| | | | acetylmannosamine kinase | | | | |
| GNRHR2 | 4.67 | 3.52 | gonadotropin-releasing | 114814 | NM_057163 | Hs.356873 | ENSG00000211451 |
| | | | hormone (type 2) receptor 2, | | | | |
| | | | pseudogene | | | | |
| GOLGA2 | 2.53 | 2.02 | golgin A2 | 2801 | NM_004486 | Hs.155827 | ENSG00000167110 |
| GOLGA6L22 | 5.03 | 3.86 | golgin A6 family-like 22 | 440243 | NM_001271664 | | ENSG00000274404 |
| GOLGA6L6 | 4.6 | 3.28 | golgin A6 family-like 6 | 727832 | NM_001145004 | Hs.569472 | ENSG00000277322 |
| GOSR1 | 3.08 | 2.61 | golgi SNAP receptor | 9527 | NM_001007024 | Hs.462680 | ENSG00000108587 |
| | | | complex member 1 | | | | |
| GPR1-AS | 4.67 | 3.49 | GPR1 antisense RNA | 101669 764 | NR_104359 | Hs.574781 | ENSG00000279220 |
| GPR37L1 | 4.34 | 3.11 | G protein-coupled receptor | 9283 | NM_004767 | Hs.132049 | ENSG00000170075 |
| | | | 37 like 1 | | | | |
| GPR82 | 3.34 | 3.21 | G protein-coupled receptor 82 | 27197 | NM_080817 | Hs.567457 | ENSG00000171657 |
| GREB1 | 4.84 | 3.69 | growth regulation by | 9687 | NM_014668 | Hs.467733 | ENSG00000196208 |
| | | | estrogen in breast cancer 1 | | | | |
| GRTP1 | 3.91 | 3.25 | growth hormone regulated | 79774 | NM_001286732 | Hs.745043 | ENSG00000139835 |
| | | | TBC protein 1 | | | | |
| GSDMA | 3.59 | 3.02 | gasdermin A | 284110 | NM_178171 | Hs.448873 | ENSG00000167914 |
| GSG1 | 4.38 | 3.34 | germ cell associated 1 | 83445 | NM_001080554 | Hs.240053 | ENSG00000111305 |
| GSTM3 | 3.45 | 2.8 | glutathione S-transferase mu | 2947 | NM_000849 | Hs.2006 | ENSG00000134202 |
| | | | 3 (brain) | | | | |
| GTF2E1 | 3.49 | 2.48 | general transcription factor | 2960 | NM_005513 | Hs.445272 | ENSG00000153767 |
| | | | IIE, polypeptide 1, alpha | | | | |
| | | | 56 kDa | | | | |
| GTF2H2 | 2.46 | 2.1 | general transcription factor | 2966 | NM_001515 | Hs.191356 | ENSG00000145736 |
| | | | IIH, polypeptide 2, 44 kDa | | | | |
| GUCA1B | 4.5 | 3.03 | guanylate cyclase activator | 2979 | NM_002098 | Hs.446529 | ENSG00000112599 |
| | | | 1B (retina) | | | | |
| GUSBP3 | 2.42 | 2.31 | glucuronidase, beta | 653188 | NR_027386 | Hs.631974 | ENSG00000253203 |
| | | | pseudogene 3 | | | | |
| HIFX-AS1 | 2.98 | 2.44 | H1FX antisense RNA 1 | 339942 | NM_001025468 | Hs.450096 | |
| HCAR1 | 3.99 | 3.68 | hydroxycarboxylic acid | 27198 | NM_032554 | Hs.610873 | ENSG00000196917 |
| | | | receptor 1 | | | | |
| HEATR5A | 2.38 | 2.18 | HEAT repeat containing 5A | 25938 | NM_015473 | Hs.744979 | ENSG00000129493 |
| HES2 | 4.11 | 3.44 | hes family bHLH | 54626 | NM_019089 | Hs.118727 | ENSG00000069812 |
| | | | transcription factor 2 | | | | |
| HHLA2 | 2.46 | 2.41 | HERV-H LTR-associating 2 | 11148 | NM_001282556 | Hs.225968 | ENSG00000114455 |
| HILPDA | 3.18 | 2.83 | hypoxia inducible lipid | 29923 | NM_001098786 | Hs.706124 | ENSG00000135245 |
| | | | droplet-associated | | | | |
| HIPK1-AS1 | 2.57 | 2.41 | HIPK1 antisense RNA 1 | 101928 846 | NR_110725 | Hs.232534 | ENSG00000235527 |
| HMGB3P1 | 4.77 | 4.13 | high mobility group box 3 | 128872 | NR_002165 | Hs.558624 | |
| | | | pseudogene 1 | | | | |
| HNF1A-AS1 | 4.5 | 3.36 | HNF1A antisense RNA 1 | 283460 | NR_024345 | Hs.612351 | ENSG00000241388 |
| HOGA1 | 4.32 | 3.68 | 4-hydroxy-2-oxoglutarate | 112817 | NM_001134670 | Hs.180346 | ENSG00000241935 |
| | | | aldolase 1 | | | | |
| HP090255 | 3.99 | 3.31 | uncharacterized | 100652 929 | NR_109783 | Hs.559249 | ENSG00000267719 |
| | | | LOC100652929 | | | | |
| HPSE | 2.05 | 1.79 | heparanase | 10855 | NM_001098540 | Hs.44227 | ENSG00000173083 |
| HSD17B13 | 4.35 | 3.67 | hydroxysteroid (17-beta) | 345275 | NM_001136230 | Hs.284414 | ENSG00000170509 |
| | | | dehydrogenase 13 | | | | |
| HSP90AB4P | 4.23 | 3.1 | heat shock protein 90 kDa | 664618 | NR_002927 | Hs.670224 | |
| | | | alpha (cytosolic), class B | | | | |
| | | | member 4, pseudogene | | | | |
| HTRA4 | 4.59 | 3.31 | HtrA serine peptidase 4 | 203100 | NM_153692 | Hs.661014 | ENSG00000169495 |
| IAPP | 3.94 | 3.28 | islet amyloid polypeptide | 3375 | NM_000415 | Hs.46835 | ENSG00000121351 |
| IBA57 | 2.85 | 2.58 | IBA57 homolog, iron-sulfur | 200205 | NM_001010867 | Hs.237017 | ENSG00000181873 |
| | | | cluster assembly | | | | |
| ICA1L | 3.12 | 2.91 | islet cell autoantigen | 130026 | NM_001288622 | Hs.516629 | ENSG00000163596 |
| | | | 1, 69 kDa-like | | | | |
| IDO1 | 5.13 | 4.37 | indoleamine 2,3-dioxygenase 1 | 3620 | NM_002164 | Hs.840 | ENSG00000131203 |
| IFNLR1 | 4.24 | 3.59 | interferon, lambda receptor 1 | 163702 | NM_170743 | Hs.221375 | ENSG00000185436 |
| IFT22 | 3.1 | 2.69 | intraflagellar transport 22 | 64792 | NM_001130820 | Hs.389104 | ENSG00000128581 |
| IL10 | 4.12 | 3.83 | interleukin 10 | 3586 | NM_000572 | Hs.193717 | ENSG00000136634 |
| IL15 | 3.25 | 2.56 | interleukin 15 | 3600 | NM_00058.5 | Hs.168132 | ENSG00000164136 |
| IL17RD | 3.95 | 3.23 | interleukin 17 receptor D | 54756 | NM_017563 | Hs.150725 | ENSG00000144730 |
| INE1 | 2.81 | 2.75 | inactivation escape 1 (non- | 8552 | NM_003669 | Hs.657350 | ENSG00000224975 |
| | | | protein coding) | | | | |
| INGX | 4.56 | 3.7 | inhibitor of growth family, | 27160 | NR_002226 | Hs.721806 | ENSG00000243468 |
| | | | X-linked, pseudogene | | | | |
| INIP | 2.46 | 1.99 | INTS3 and NABP | 58493 | NM_021218 | Hs.658575 | ENSG00000148153 |
| | | | interacting protein | | | | |
| INMT | 4.25 | 3.37 | indolethylamine N- | 11185 | NM_001199219 | Hs.632629 | ENSG00000241644 |
| | | | methyltransferase | | | | |
| IPO5P1 | 2.4 | 2.1 | importin 5 pseudogene 1 | 100132 815 | NR_103741 | Hs.629249 | |
| IRGQ | 3.27 | 2.78 | immunity-related GTPase | 126298 | NM_001007561 | Hs.6217 | ENSG00000167378 |
| | | | family, Q | | | | |
| ITIH5 | 4.34 | 3.6 | inter-alpha-trypsin inhibitor | 80760 | NM_001001851 | Hs.498586 | ENSG00000123243 |
| | | | heavy chain family, member 5 | | | | |
| JPX | 3.59 | 3.15 | JPX transcript, XIST | 554203 | NR_024582 | Hs.648316 | ENSG00000225470 |
| | | | activator (non-protein | | | | |
| | | | coding) | | | | |
| KANTR | 2.83 | 2.58 | KDM5C adjacent non- | 102723 508 | NR_110456 | Hs.633244 | |
| | | | coding transcript | | | | |
| KBTBD12 | 4.43 | 3.83 | kelch repeat and BTB (POZ) | 166348 | NM_207335 | Hs.132087 | ENSG00000187715 |
| | | | domain containing 12 | | | | |
| KBTBD6 | 2.46 | 2.05 | kelch repeat and BTB (POZ) | 89890 | NM_152903 | Hs.534040 | ENSG00000165572 |
| | | | domain containing 6 | | | | |
| KCNA7 | 4.88 | 3.92 | potassium channel, voltage | 3743 | NM_031886 | Hs.306973 | ENSG00000104848 |
| | | | gated shaker related | | | | |
| | | | subfamily A, member 7 | | | | |
| KCNJ11 | 3.9 | 3.43 | potassium channel, inwardly | 3767 | NM_000525 | Hs.248141 | ENSG00000187486 |
| | | | rectifying subfamily J, | | | | |
| | | | member 11 | | | | |
| KCNJ5 | 4.19 | 3.57 | potassium channel, inwardly | 3762 | NM_000890 | Hs.444595 | ENSG00000120457 |
| | | | rectifying subfamily J, | | | | |
| | | | member 5 | | | | |
| KCNQ1OT1 | 4.08 | 3.18 | KCNQ1 opposite | 10984 | NR_002728 | Hs.604823 | ENSG00000269821 |
| | | | strand/antisense transcript 1 | | | | |
| | | | (non-protein coding) | | | | |
| KDELC | 2.63 | 2.33 | KDEL (Lys-Asp-Glu-Leu) | 143888 | NM_153705 | Hs.83286 | ENSG00000178202 |
| | | | (SEQ ID NO: 802) | | | | |
| | | | containing 2 | | | | |
| KDM4A-AS1 | 4.3 | 3.3 | KDM4A antisense RNA 1 | 100132 774 | NR_033827 | Hs.655569 | |
| KIAA0101 | 4.61 | 2.88 | KIAA0101 | 9768 | NM_001029989 | Hs.81892 | ENSG00000166803 |
| KIAA1161 | 4.87 | 3.65 | KIAA1161 | 57462 | NM_020702 | Hs.522083 | ENSG00000164976 |
| KIAA1324 | 2.42 | 2.31 | KIAA1324 | 57535 | NM_001267048 | Hs.708190 | ENSG00000116299 |
| KIAA1456 | 4.71 | 3.43 | KIAA1456 | 57604 | NM_001099677 | Hs.202521 | ENSG00000250305 |
| KIAA1614 | 3.66 | 2.84 | KIAA1614 | 57710 | NM_020950 | Hs.734816 | ENSG00000135835 |
| KIAA1919 | 3.49 | 2.61 | KIAA1919 | 91749 | NM_153369 | Hs.400572 | ENSG00000173214 |
| KIF18B | 4.05 | 3.41 | kinesin family member 18B | 146909 | NM_001080443 | Hs.135094 | ENSG00000186185 |
| KIF1B | 2.07 | 1.75 | kinesin family member 1B | 23095 | NM_015074 | Hs.97858 | ENSG00000054523 |
| KIF3A | 2.18 | 1.94 | kinesin family member 3A | 11127 | NM_001300791 | Hs.43670 | ENSG00000131437 |
| KIR3DX1 | 3.74 | 3.28 | killer cell immunoglobulin- | 90011 | NM_001047605 | Hs.288520 | ENSG00000104970 |
| | | | like receptor, three domains, | | | | |
| | | | X1 | | | | |
| KLF3-AS1 | 2.34 | 2.48 | KLF3 antisense RNA 1 | 79667 | NM_024614 | Hs.29725 | ENSG00000231160 |
| KLRD1 | 2.65 | 2.36 | killer cell lectin-like receptor | 3824 | NM_001114396 | Hs.562457 | ENSG00000134539 |
| | | | subfamily D, member 1 | | | | |
| KREMEN1 | 4.81 | 3.4 | kringle containing | 83999 | NM_001039570 | Hs.229335 | ENSG00000183762 |
| | | | transmembrane protein 1 | | | | |
| KRT18 | 2.8 | 2.75 | keratin 18, type I | 3875 | NM_000224 | Hs.406013 | ENSG00000111057 |
| KRT8 | 3.45 | 2.95 | keratin 8, type II | 3856 | NM_001256282 | Hs.533782 | ENSG00000170421 |
| L1TD1 | 4.64 | 3.67 | LINE-1 type transposase | 54596 | NM_001164835 | Hs.685462 | ENSG00000240563 |
| | | | domain containing 1 | | | | |
| L2HGDH | 4.91 | 3.54 | L-2-hydroxyglutarate | 79944 | NM_024884 | Hs.256034 | ENSG00000087299 |
| | | | dehydrogenase | | | | |
| LAIR1 | 2.87 | 2.24 | leukocyte-associated | 3903 | NM_001289023 | Hs.572535 | ENSG00000167613 |
| | | | immunoglobulin-like | | | | |
| | | | receptor 1 | | | | |
| LARS2-AS1 | 3.66 | 2.87 | LARS2 antisense RNA 1 | 100885 795 | NR_048543 | Hs.641094 | |
| LDLR | 2.41 | 2.26 | low density lipoprotein | 3949 | NM_000527 | Hs.213289 | ENSG00000130164 |
| | | | receptor | | | | |
| LGMN | 3.31 | 2.67 | legumain | 5641 | NM_001008530 | Hs.18069 | ENSG00000100600 |
| LIFR-AS1 | 5.36 | 3.4 | LIFR antisense RNA 1 | 100506 495 | NR_103553 | Hs.657602 | ENSG00000244968 |
| LINC00092 | 2.59 | 2.33 | long intergenic non-protein | 100188 953 | NR_024129 | Hs.434310 | ENSG00000225194 |
| | | | coding RNA 92 | | | | |
| LINC00260 | 2.91 | 2.68 | long intergenic non-protein | 84719 | NM_032633 | Hs.661178 | |
| | | | coding RNA 260 | | | | |
| LINC00294 | 3.93 | 3.15 | long intergenic non-protein | 283267 | NR_015451 | Hs.533701 | ENSG00000280798 |
| | | | coding RNA 294 | | | | |
| LINC00311 | 3.85 | 3.74 | long intergenic non-protein | 197196 | NM_153238 | Hs.679002 | ENSG00000179219 |
| | | | coding RNA 311 | | | | |
| LINC00346 | 5.19 | 3.87 | long intergenic non-protein | 283487 | NM_178514 | Hs.245390 | ENSG00000255874 |
| | | | coding RNA 346 | | | | |
| LINC00371 | 6.06 | 4.54 | long intergenic non-protein | 647166 | NR_102431 | Hs.195052 | |
| | | | coding RNA 371 | | | | |
| LINC00381 | 4.68 | 3.4 | long intergenic non-protein | 100874 151 | NR_047005 | Hs.564552 | ENSG00000226240 |
| | | | coding RNA 381 | | | | |
| LINC00458 | 5.94 | 4.75 | long intergenic non-protein | 100507 428 | NR_108062 | Hs.351262 | ENSG00000234787 |
| | | | coding RNA 458 | | | | |
| LINC00470 | 3.85 | 2.95 | long intergenic non-protein | 56651 | NM_031416 | Hs.541165 | |
| | | | coding RNA 470 | | | | |
| LINC00483 | 3.52 | 3.19 | long intergenic non-protein | 55018 | NM_017928 | Hs.389460 | ENSG00000167117 |
| | | | coding RNA 483 | | | | |
| LINC00485 | 7.11 | 5.02 | long intergenic non-protein | 283432 | NR_033855 | Hs.382110 | ENSG00000258169 |
| | | | coding RNA 485 | | | | |
| LINC00501 | 4.51 | 4.36 | long intergenic non-protein | 100820 709 | NR_047465 | Hs.518409 | ENSG00000203645 |
| | | | coding RNA 501 | | | | |
| LINC00506 | 4.36 | 3.32 | long intergenic non-protein | 100846 978 | NR_047469 | Hs.570649 | ENSG00000281392 |
| | | | coding RNA 506 | | | | |
| LINC00507 | 5.42 | 4.12 | long intergenic non-protein | 100862 680 | NR_046392 | Hs.385496 | ENSG00000256193 |
| | | | coding RNA 507 | | | | |
| LINC00547 | 5.74 | 4.09 | long intergenic non-protein | 400121 | NR_040244 | Hs.558894 | ENSG00000275226 |
| | | | coding RNA 547 | | | | |
| LINC00578 | 4.72 | 3.66 | long intergenic non-protein | 100505 566 | NR_047568 | Hs.581170 | |
| | | | coding RNA 578 | | | | |
| LINC00620 | 5.09 | 3.5 | long intergenic non-protein | 285375 | NR_027103 | Hs.319969 | ENSG00000224514 |
| | | | coding RNA 620 | | | | |
| LINC00649 | 2.97 | 2.45 | long intergenic non-protein | 400863 | NM_001288961 | Hs.729814 | ENSG00000237945 |
| | | | coding RNA 649 | | | | |
| LINC00652 | 4.41 | 3.52 | long intergenic non-protein | 29075 | NM_014162 | Hs.584899 | |
| | | | coding RNA 652 | | | | |
| LINC00663 | 4.14 | 3.4 | long intergenic non-protein | 284440 | NR_026956 | Hs.665307 | |
| | | | coding RNA 663 | | | | |
| LINC00665 | 3.09 | 2.84 | long intergenic non-protein | 100506 930 | NR_038278 | Hs.595153 | ENSG00000232677 |
| | | | coding RNA 665 | | | | |
| LINC00670 | 4.75 | 3.48 | long intergenic non-protein | 284034 | NR_034144 | Hs.376614 | ENSG00000179136 |
| | | | coding RNA 670 | | | | |
| LINC00672 | 3.85 | 3 | long intergenic non-protein | 100505 576 | NR_038847 | Hs.634043 | ENSG00000263874 |
| | | | coding RNA 672 | | | | |
| LINC00678 | 5.57 | 3.89 | long intergenic non-protein | 101410 541 | NR_102708 | Hs.471439 | ENSG00000254934 |
| | | | coding RNA 678 | | | | |
| LINC00889 | 5.99 | 4.42 | long intergenic non-protein | 158696 | NR_026935 | Hs.558664 | |
| | | | coding RNA 889 | | | | |
| LINC00907 | 3.95 | 3.12 | long intergenic non-protein | 284260 | NR_046174 | Hs.652819 | ENSG00000267586 |
| | | | coding RNA 907 | | | | |
| LINC00910 | 3 | 2.48 | long intergenic non-protein | 100130 581 | NR_027412 | Hs.546897 | ENSG00000188825 |
| | | | coding RNA 910 | | | | |
| LINC00923 | 2.84 | 2.56 | long intergenic non-protein | 91948 | NR_024172 | Hs.130423 | ENSG00000251209 |
| | | | coding RNA 923 | | | | |
| LINC00924 | 4.4 | 3.05 | long intergenic non-protein | 145820 | NR_027132 | Hs.652702 | ENSG00000259134 |
| | | | coding RNA 924 | | | | |
| LINC00941 | 3.79 | 3.38 | long intergenic non-protein | 100287 314 | NR_040245 | Hs.355210 | ENSG00000235884 |
| | | | coding RNA 941 | | | | |
| LINC00958 | 4.91 | 3.92 | long intergenic non-protein | 100506 305 | NR_038904 | Hs.153408 | ENSG00000251381 |
| | | | coding RNA 958 | | | | |
| LINC00963 | 2.8 | 2.51 | long intergenic non-protein | 100506 190 | NR_038955 | Hs.529860 | |
| | | | coding RNA 963 | | | | |
| LINC00965 | 4.43 | 3.51 | long intergenic non-protein | 349196 | NM_001025473 | Hs.559040 | |
| | | | coding RNA 965 | | | | |
| LINC00970 | 4.24 | 3.32 | long intergenic non-protein | 101978 719 | NR_104091 | Hs.517849 | ENSG00000203601 |
| | | | coding RNA 970 | | | | |
| LINC01012 | 3.13 | 2.69 | long intergenic non-protein | 100507 173 | NR_038292 | Hs.635987 | ENSG00000281706 |
| | | | coding RNA 1012 | | | | |
| LINC01021 | 6.93 | 4.93 | long intergenic non-protein | 643401 | NR_038848 | Hs.533212 | ENSG00000250337 |
| | | | coding RNA 1021 | | | | |
| LINC01057 | 5.48 | 3.49 | long intergenic non-protein | 101928 079 | NR_104131 | Hs.596857 | ENSG00000224081 |
| | | | coding RNA 1057 | | | | |
| LINC01087 | 5.01 | 3.44 | long intergenic non-protein | 101927 994 | NR_108087 | Hs.635757 | ENSG00000224559 |
| | | | coding RNA 1087 | | | | |
| LINC01099 | 4.33 | 3.58 | long intergenic non-protein | 101928 656 | NR_108092 | Hs.508131 | ENSG00000251504 |
| | | | coding RNA 1099 | | | | |
| LINC01160 | 4.12 | 3.22 | long intergenic non-protein | 100129 269 | NR_034126 | Hs.689728 | |
| | | | coding RNA 1160 | | | | |
| LINC01204 | 4.02 | 3.25 | long intergenic non-protein | 101927 528 | NR_104644 | Hs.550772 | ENSG00000229563 |
| | | | coding RNA 1204 | | | | |
| LINC01205 | 4.55 | 3.45 | long intergenic non-protein | 401082 | NM_001145553 | Hs.477089 | ENSG00000228980 |
| | | | coding RNA 1205 | | | | |
| LINC01207 | 4.05 | 3.59 | long intergenic non-protein | 100505 989 | NR_038834 | Hs.328236 | ENSG00000248771 |
| | | | coding RNA 1207 | | | | |
| LINC01209 | 4.74 | 3.4 | long intergenic non-protein | 101928 684 | NR_110819 | Hs.639352 | ENSG00000228308 |
| | | | coding RNA 1209 | | | | |
| LINC01212 | 3.81 | 3.27 | long intergenic non-protein | 101927 152 | NR_110000 | Hs.382046 | ENSG00000240405 |
| | | | coding RNA 1212 | | | | |
| LINC01226 | 4.31 | 3.34 | long intergenic non-protein | 284551 | NR_027085 | Hs.658659 | ENSG00000223907 |
| | | | coding RNA 1226 | | | | |
| LINC01247 | 4.93 | 3.53 | long intergenic non-protein | 101929 390 | NR_110251 | Hs.434407 | ENSG00000227007 |
| | | | coding RNA 1247 | | | | |
| LINC01252 | 4.11 | 2.85 | long intergenic non-protein | 338817 | NR_033890 | Hs.733066 | ENSG00000247157 |
| | | | coding RNA 1252 | | | | |
| LINC01299 | 3.88 | 3.19 | long intergenic non-protein | 286186 | NR_033893 | Hs.449427 | ENSG00000254081 |
| | | | coding RNA 1299 | | | | |
| LINC01356 | 4.31 | 3.22 | long intergenic non-protein | 100996 702 | NR_103746 | Hs.632431 | ENSG00000215866 |
| | | | coding RNA 1356 | | | | |
| LOC100128233 | 4.52 | 3.88 | uncharacterized | 100128 233 | NR_103769 | Hs.497323 | ENSG00000255002 |
| | | | LOC100128233 | | | | |
| LOC100128288 | 4.28 | 3.38 | uncharacterized | 100128 288 | NR_0244 | Hs.549913 | |
| | | | LOC100128288 | | | | |
| LOC100128398 | 3.21 | 2.39 | uncharacterized | 100128 398 | NR_036508 | Hs.655081 | ENSG00000176593 |
| | | | LOC100128398 | | | | |
| LOC100128531 | 3.85 | 2.9 | uncharacterized | 100128 531 | NR_038941 | Hs.662126 | ENSG00000203280 |
| | | | LOC100128531 | | | | |
| LOC100128573 | 2.46 | 2.68 | uncharacterized | 100128 573 | NR_024491 | Hs.465761 | |
| | | | LOC100128573 | | | | |
| LOC100129940 | 3.73 | 3.44 | uncharacterized | 100129 940 | NM_001292023 | Hs.685856 | ENSG00000197301 |
| | | | LOC100129940 | | | | |
| LOC100130451 | 4.59 | 3.55 | uncharacterized | 100130 451 | NM_001242575 | | |
| | | | LOC100130451 | | | | |
| LOC100131257 | 4.35 | 3.29 | zinc finger protein 655 | 100131 257 | NR_034022 | Hs.551110 | |
| | | | pseudogene | | | | |
| LOC100131564 | 2.81 | 2.26 | uncharacterized | 100131 564 | NR_034089 | Hs.732666 | |
| | | | LOC100131564 | | | | |
| LOC100131626 | 4.21 | 3.02 | uncharacterized | 100131 626 | NR_046369 | Hs.721614 | |
| | | | LOC100131626 | | | | |
| LOC100132077 | 3.76 | 3.1 | uncharacterized | 100132 077 | NR_033937 | Hs.679111 | ENSG00000232063 |
| | | | LOC100132077 | | | | |
| LOC100190986 | 2.12 | 2.25 | uncharacterized | 100190 986 | NR_024456 | Hs.648439 | |
| | | | LOC100190986 | | | | |
| LOC100268168 | 4 | 3.55 | uncharacterized | 100268 168 | NR_026682 | Hs.519766 | ENSG00000204758 |
| | | | LOC100268168 | | | | |
| LOC100287015 | 3.01 | 2.93 | uncharacterized | 100287 015 | NR_040040 | Hs.156928 | ENSG00000246089 |
| | | | LOC100287015 | | | | |
| LOC100287042 | 2.11 | 1.98 | uncharacterized | 100287 042 | NR_036520 | Hs.514470 | ENSG00000263843 |
| | | | LOC100287042 | | | | |
| LOC100287792 | 3.43 | 3.04 | uncharacterized | 100287 792 | NM_001001690 | Hs.517026 | ENSG00000204117 |
| | | | LOC100287792 | | | | |
| LOC100287846 | 4.08 | 2.69 | patched 1 pseudogene | 100287 846 | NR_037168 | Hs.21550 | |
| LOC100335030 | 4.83 | 3.91 | FGFR1 oncogene partner 2 | 100335 030 | NR_033257 | Hs.687044 | |
| | | | pseudogene | | | | |
| LOC100420587 | 5.27 | 3.7 | SHC SH2-domain binding | 100420 587 | NR_110759 | Hs.569956 | ENSG00000267243 |
| | | | protein 1 pseudogene | | | | |
| LOC100506023 | 3.79 | 2.76 | uncharacterized | 100506 023 | NR_037845 | Hs.731284 | |
| | | | LOC100506023 | | | | |
| LOC100506083 | 3.67 | 3.08 | uncharacterized | 100506 083 | NR_039997 | Hs.635008 | ENSG00000261777 |
| | | | LOC100506083 | | | | |
| LOC100506127 | 3.73 | 3.1 | putative uncharacterized | 100506 127 | NM_001013634 | Hs.503319 | ENSG00000179240 |
| | | | protein FLJ37770-like | | | | |
| LOC100506472 | 3.36 | 2.68 | uncharacterized | 100506 472 | NR_040535 | Hs.729080 | |
| | | | LOC100506472 | | | | |
| LOC100506551 | 4.19 | 3.53 | uncharacterized | 100506 551 | NR_103809 | Hs.657861 | ENSG00000257279 |
| | | | LOC100506551 | | | | |
| LOC100506688 | 4.09 | 3.23 | uncharacterized | 100506 688 | NM_001242737 | Hs.532063 | ENSG00000215246 |
| | | | LOC100506688 | | | | |
| LOC100506746 | 3.32 | 2.75 | uncharacterized | 100506 746 | NR_038841 | Hs.657766 | ENSG00000163633 |
| | | | LOC100506746 | | | | |
| LOC100506990 | 2.84 | 2.36 | uncharacterized | 100506 990 | NR_040091 | Hs.656893 | |
| | | | LOC100506990 | | | | |
| LOC100996251 | 4 | 3.37 | uncharacterized | 100996 251 | NR_103777 | Hs.382067 | ENSG00000238198 |
| | | | LOC100996251 | | | | |
| LOC101409256 | 3.94 | 3.49 | cell division cycle 42 | 101409 256 | NR_102424 | | |
| | | | pseudogene | | | | |
| LOC101926889 | 4.24 | 3.31 | uncharacterized | 101926 889 | NR_109994 | Hs.585997 | |
| | | | LOC101926889 | | | | |
| LOC101927181 | 2.82 | 2.67 | uncharacterized | 101927 181 | NR_108066 | Hs.288853 | ENSG00000136213 |
| | | | LOC101927181 | | | | |
| LOC101927257 | 3.78 | 3.16 | uncharacterized | 101927 257 | NR_109965 | Hs.662725 | ENSG00000232564 |
| | | | LOC101927257 | | | | |
| LOC101927274 | 4.46 | 3.67 | uncharacterized | 101927 274 | NR_110751 | Hs.591168 | ENSG00000249383 |
| | | | LOC101927274 | | | | |
| LOC101927374 | 4.86 | 3.64 | uncharacterized | 101927 374 | NR_110133 | Hs.570644 | |
| | | | LOC101927374 | | | | |
| LOC101927415 | 3.2 | 2.84 | uncharacterized | 101927 415 | NR_110049 | Hs.636524 | |
| | | | LOC101927415 | | | | |
| LOC101927476 | 4.99 | 4.19 | uncharacterized | 101927 476 | NR_110386 | Hs.522607 | ENSG00000236393 |
| | | | LOC101927476 | | | | |
| LOC101927575 | 4.56 | 3.2 | uncharacterized | 101927 575 | NR_110995 | Hs.459826 | ENSG00000227463 |
| | | | LOC101927575 | | | | |
| LOC101927740 | 4.04 | 3.36 | uncharacterized | 101927 740 | NR_109890 | Hs.738721 | ENSG00000245812 |
| | | | LOC101927740 | | | | |
| LOC101927797 | 3.21 | 2.79 | uncharacterized | 101927 797 | NR_109925 | Hs.551743 | |
| | | | LOC101927797 | | | | |
| LOC101927884 | 5.21 | 3.69 | uncharacterized | 101927 884 | NR_110281 | Hs.671110 | ENSG00000231172 |
| | | | LOC101927884 | | | | |
| LOC101928103 | 4.63 | 3.08 | uncharacterized | 101928 103 | NR_110292 | Hs.665619 | ENSG00000229267 |
| | | | LOC101928103 | | | | |
| LOC101928137 | 4.58 | 3.44 | uncharacterized | 101928 137 | NR_110130 | Hs.694666 | ENSG00000258123 |
| | | | LOC101928137 | | | | |
| LOC101928254 | 4.24 | 4.15 | uncharacterized | 101928 254 | NR_110182 | Hs.571236 | ENSG00000219445 |
| | | | LOC101928254 | | | | |
| LOC101928303 | 4.56 | 3.27 | uncharacterized | 101928 303 | NR_110698 | Hs.375067 | ENSG00000236155 |
| | | | LOC101928303 | | | | |
| LOC101928336 | 4.87 | 3.73 | uncharacterized | 101928 336 | NR_110396 | | ENSG00000230392 |
| | | | LOC101928336 | | | | |
| LOC101928372 | 3.85 | 3.11 | uncharacterized | 101928 372 | NR_110695 | | ENSG00000198358 |
| | | | LOC101928372 | | | | |
| LOC101928401 | 3.63 | 3.01 | uncharacterized | 101928 401 | NR_108099 | Hs.385614 | ENSG00000233288 |
| | | | LOC101928401 | | | | |
| LOC101928495 | 5.19 | 3.89 | uncharacterized | 101928 495 | NR_110409 | Hs.545998 | ENSG00000237208 |
| | | | LOC101928495 | | | | |
| LOC101928514 | 5.14 | 3.96 | uncharacterized | 101928 514 | NR_110837 | Hs.617206 | ENSG00000267065 |
| | | | LOC101928514 | | | | |
| LOC101928567 | 4.39 | 3.45 | uncharacterized | 101928 567 | NR_110839 | Hs.569757 | ENSG00000237057 |
| | | | LOC101928567 | | | | |
| LOC101928580 | 3.93 | 3.68 | uncharacterized | 101928 580 | NR_120556 | Hs.569025 | ENSG00000246211 |
| | | | LOC101928580 | | | | |
| LOC101928597 | 4.26 | 3.35 | uncharacterized | 101928 597 | NR_110091 | Hs.638942 | ENSG00000246394 |
| | | | LOC101928597 | | | | |
| LOC101928600 | 4.9 | 3.96 | uncharacterized | 101928 600 | NR_109904 | Hs.694699 | ENSG00000250127 |
| | | | LOC101928600 | | | | |
| LOC101928738 | 3.84 | 3.53 | uncharacterized | 101928 738 | NR_110851 | Hs.399280 | ENSG00000262188 |
| | | | LOC101928738 | | | | |
| LOC101928936 | 4.73 | 3.78 | uncharacterized | 101928 936 | NR_110867 | Hs.533080 | |
| | | | LOC101928936 | | | | |
| LOC101929181 | 3.42 | 2.44 | uncharacterized | 101929 181 | NR_104624 | Hs.568616 | ENSG00000235643 |
| | | | LOC101929181 | | | | |
| LOC101929224 | 4.44 | 3.84 | uncharacterized | 101929 224 | NR_110787 | Hs.639369 | ENSG00000260088 |
| | | | LOC101929224 | | | | |
| LOC101929259 | 4.17 | 3.67 | uncharacterized | 101929 259 | NR_120424 | Hs.638490 | |
| | | | LOC101929259 | | | | |
| LOC101929486 | 4.25 | 3.06 | uncharacterized | 101929 486 | NR_109868 | Hs.548761 | ENSG00000233048 |
| | | | LOC101929486 | | | | |
| LOC101929567 | 4.72 | 3.61 | uncharacterized | 101929 567 | NR_11027 | Hs.634706 | ENSG00000236008 |
| | | | LOC101929567 | | | | |
| LOC101929586 | 4.34 | 3.59 | uncharacterized | 101929 586 | NR_120363 | Hs.569426 | ENSG00000259175 |
| | | | LOC101929586 | | | | |
| LOC101929698 | 3.64 | 2.61 | uncharacterized | 101929 698 | NR_110619 | Hs.638392 | ENSG00000277301 |
| | | | LOC101929698 | | | | |
| LOC102467081 | 4.99 | 3.91 | uncharacterized | 102467 081 | NR_104662 | | |
| | | | LOC102467081 | | | | |
| LOC102723769 | 4.8 | 3.53 | uncharacterized | 102723 769 | NR_110761 | Hs.652926 | |
| | | | LOC102723769 | | | | |
| LOC102724927 | 4.39 | 3.7 | uncharacterized | 102724 927 | NR_120311 | Hs.364739 | ENSG00000262185 |
| | | | LOC102724927 | | | | |
| LOC143666 | 2.94 | 2.59 | uncharacterized | 143666 | NR_026967 | Hs.337054 | |
| | | | LOC143666 | | | | |
| LOC150935 | 4.82 | 4.54 | uncharacterized | 150935 | NR_037808 | Hs.555582 | |
| | | | LOC150935 | | | | |
| LOC151475 | 3.63 | 3.2 | uncharacterized | 151475 | NR_040038 | Hs.528154 | ENSG00000226125 |
| | | | LOC151475 | | | | |
| LOC257396 | 3.45 | 2.42 | uncharacterized | 257396 | NR_034107 | Hs.12326 | ENSG00000247796 |
| | | | LOC257396 | | | | |
| LOC283683 | 4.2 | 4 | uncharacterized | 283683 | NR_040057 | Hs.534616 | ENSG00000274253 |
| | | | LOC283683 | | | | |
| LOC284023 | 3.54 | 2.88 | uncharacterized | 284023 | NR_024349 | Hs.744470 | ENSG00000179859 |
| | | | LOC284023 | | | | |
| LOC284379 | 4.31 | 3.51 | solute carrier family 7 | 284379 | NR_002938 | Hs.631571 | ENSG00000268864 |
| | | | (cationic amino acid | | | | |
| | | | transporter, y+ system), | | | | |
| | | | member 3 pseudogene | | | | |
| LOC284412 | 6.66 | 4.68 | uncharacterized | 284412 | NR_029390 | Hs.635932 | |
| | | | LOC284412 | | | | |
| LOC284454 | 4.32 | 3.54 | uncharacterized | 284454 | NR_036515 | Hs.436426 | ENSG00000267519 |
| | | | LOC284454 | | | | |
| LOC284581 | 4.12 | 3.17 | uncharacterized | 284581 | NR_046097 | | |
| | | | LOC284581 | | | | |
| LOC284865 | 4.37 | 3.67 | uncharacterized | 284865 | NR_038460 | Hs.638498 | ENSG00000249923 |
| | | | LOC284865 | | | | |
| LOC284950 | 4.2 | 3.63 | uncharacterized | 284950 | NR_038888 | Hs.570227 | |
| | | | LOC284950 | | | | |
| LOC285696 | 4.41 | 3.57 | uncharacterized | 285696 | NM_173669 | Hs.646924 | ENSG00000215196 |
| | | | LOC285696 | | | | |
| LOC286437 | 4.49 | 3.29 | uncharacterized | 286437 | NR_039980 | Hs.656786 | |
| | | | LOC286437 | | | | |
| LOC339166 | 3.75 | 2.65 | uncharacterized | 339166 | NR_040000 | Hs.736088 | ENSG00000179314 |
| | | | LOC339166 | | | | |
| LOC339803 | 3.45 | 2.76 | uncharacterized | 339803 | NR_036496 | Hs.252433 | ENSG00000212978 |
| | | | LOC339803 | | | | |
| LOC389641 | 3.53 | 2.91 | uncharacterized | 389641 | NR_033928 | Hs.591835 | ENSG00000246582 |
| | | | LOC389641 | | | | |
| LOC400958 | 4.62 | 3.57 | uncharacterized | 400958 | NR_036586 | Hs.591565 | ENSG00000237638 |
| | | | LOC400958 | | | | |
| LOC401052 | 4.04 | 3.52 | uncharacterized | 401052 | NM_001008737 | Hs.662766 | |
| | | | LOC401052 | | | | |
| LOC440173 | 5.21 | 3.95 | uncharacterized | 440173 | NR_027471 | Hs.127361 | ENSG00000269994 |
| | | | LOC440173 | | | | |
| LOC440300 | 3.9 | 3.42 | chondroitin sulfate | 440300 | NR_033738 | Hs.546565 | ENSG00000259295 |
| | | | proteoglycan 4 pseudogene | | | | |
| LOC441242 | 2.11 | 2.07 | uncharacterized | 441242 | NM_001013464 | Hs.373941 | ENSG00000272693 |
| | | | LOC441242 | | | | |
| LOC643406 | 4.43 | 3.27 | uncharacterized | 643406 | NM_175877 | Hs.431161 | |
| | | | LOC643406 | | | | |
| LOC644919 | 4.98 | 3.81 | uncharacterized | 644919 | NR_109757 | Hs.434414 | |
| | | | LOC644919 | | | | |
| LOC646214 | 4.3 | 3.38 | p21 protein (Cdc42/Rac)- | 646214 | NR_027053 | Hs.510697 | |
| | | | activated kinase 2 | | | | |
| | | | pseudogene | | | | |
| LOC650293 | 6.38 | 3.67 | seven transmembrane helix | 650293 | NM_001040071 | Hs.535167 | |
| | | | receptor | | | | |
| LOC727896 | 3.8 | 2.72 | cysteine and histidine-rich | 727896 | NR_026659 | Hs.673126 | |
| | | | domain (CHORD) | | | | |
| | | | containing 1 pseudogene | | | | |
| LOC728613 | 2.3 | 2.03 | programmed cell death 6 | 728613 | NR_003713 | Hs.720393 | |
| | | | pseudogene | | | | |
| LOC728752 | 4.03 | 3.31 | uncharacterized | 728752 | NR_036504 | Hs.729762 | ENSG00000267309 |
| | | | LOC728752 | | | | |
| LOC729603 | 4.36 | 3.16 | calcineurin-like EF-hand | 729603 | NR_003288 | Hs.674810 | ENSG00000213073 |
| | | | protein 1 pseudogene | | | | |
| LOC729732 | 3.63 | 2.93 | uncharacterized | 729732 | NR_047662 | Hs.322761 | |
| | | | LOC729732 | | | | |
| LOC729987 | 4.36 | 3.05 | uncharacterized | 729987 | NR_046088 | Hs.683961 | ENSG00000226053 |
| | | | LOC729987 | | | | |
| LOC731424 | 4.17 | 3.03 | uncharacterized | 731424 | NR_037867 | Hs.427740 | |
| | | | LOC731424 | | | | |
| LOH12CR2 | 4.49 | 3.39 | loss of heterozygosity, 12, | 503693 | NR_024061 | Hs.67553 | ENSG00000205791 |
| | | | chromosomal region 2 (non- | | | | |
| | | | protein coding) | | | | |
| LPAL2 | 3.58 | 2.94 | lipoprotein, Lp(a)-like 2, | 80350 | NM_024492 | Hs.654503 | ENSG00000213071 |
| | | | pseudogene | | | | |
| LPCAT2 | 3.36 | 2.61 | lysophosphatidylcholine | 54947 | NM_017839 | Hs.460857 | ENSG00000087253 |
| | | | acyltransferase 2 | | | | |
| LPP | 2.85 | 2.51 | LIM domain containing | 4026 | NM_001167671 | Hs.720220 | ENSG00000145012 |
| | | | preferred translocation | | | | |
| | | | partner in lipoma | | | | |
| LRPAP1 | 2.05 | 1.91 | low density lipoprotein | 4043 | NM_002337 | Hs.40966 | ENSG00000163956 |
| | | | receptor-related protein | | | | |
| | | | associated protein 1 | | | | |
| LRRC27 | 3.6 | 2.88 | leucine rich repeat | 80313 | NM_001143757 | Hs.119897 | ENSG00000148814 |
| | | | containing 27 | | | | |
| LRRC57 | 3.77 | 3.17 | leucine rich repeat | 255252 | NM_153260 | Hs.234681 | ENSG00000180979 |
| | | | containing 57 | | | | |
| LRRN4CL | 4.42 | 4.03 | LRRN4 C-terminal like | 221091 | NM_203422 | Hs.427449 | ENSG00000177363 |
| LRTOMT | 4.01 | 3.25 | leucine rich transmembrane | 220074 | NM_001145307 | Hs.317243 | ENSG00000184114 |
| | | | and O-methyltransferase | | | | |
| | | | domain containing | | | | |
| LUCAT1 | 5.28 | 4.95 | lung cancer associated | 100505 994 | NR_103548 | Hs.628363 | ENSG00000248323 |
| | | | transcript 1 (non-protein | | | | |
| | | | coding) | | | | |
| LYRM7 | 2.35 | 2.03 | LYR motif containing 7 | 90624 | NM_001293735 | Hs.115467 | ENSG00000186687 |
| MAB21L3 | 4.04 | 3.19 | mab-21-like 3 (C. elegans) | 126868 | NM_152367 | Hs.376194 | ENSG00000173212 |
| MAGEA10 | 3.73 | 3.52 | melanoma antigen family | 4109 | NM_001011543 | Hs.18048 | ENSG00000124260 |
| | | | A10 | | | | |
| MAN1B1-AS1 | 2.93 | 2.7 | MAN1B1 antisense RNA 1 | 100289 341 | NR_027447 | Hs.593896 | ENSG00000268996 |
| | | | (head to head) | | | | |
| MANEAL | 6.55 | 4.78 | mannosidase, endo-alpha- | 149175 | NM_001031740 | Hs.534562 | ENSG00000185090 |
| | | | like | | | | |
| MAPILC3C | 5.17 | 3.96 | microtubule-associated | 440738 | NM_001004343 | Hs.534971 | ENSG00000197769 |
| | | | protein 1 light chain 3 | | | | |
| | | | gamma | | | | |
| MAP3K13 | 2.6 | 2.25 | mitogen-activated protein | 9175 | NM_001242314 | Hs.591306 | ENSG00000073803 |
| | | | kinase kinase kinase 13 | | | | |
| MAP7D3 | 2.78 | 2.32 | MAP7 domain containing 3 | 79649 | NM_001173516 | Hs.446275 | ENSG00000129680 |
| MARVELD3 | 4.25 | 3.45 | MARVEL domain | 91862 | NM_001017967 | Hs.513706 | ENSG00000140832 |
| | | | containing 3 | | | | |
| MBOAT1 | 4.45 | 3.23 | membrane bound O- | 154141 | NM_001080480 | Hs.377830 | ENSG00000172197 |
| | | | acyltransferase domain | | | | |
| | | | containing 1 | | | | |
| MBOAT2 | 4.33 | 2.81 | membrane bound O- | 129642 | NM_138799 | Hs.467634 | ENSG00000143797 |
| | | | acyltransferase domain | | | | |
| | | | containing 2 | | | | |
| MCFD2 | 3.28 | 2.63 | multiple coagulation factor | 90411 | NM_001171506 | Hs.662152 | ENSG00000180398 |
| | | | deficiency 2 | | | | |
| MCUR1 | 2.23 | 1.92 | mitochondrial calcium | 63933 | NM_001031713 | Hs.214043 | ENSG00000050393 |
| | | | uniporter regulator 1 | | | | |
| MED15P9 | 4.39 | 3.57 | mediator complex subunit 15 | 285103 | NR_033903 | Hs.570106 | ENSG00000223760 |
| | | | pseudogene 9 | | | | |
| MED18 | 3.5 | 2.65 | mediator complex subunit 18 | 54797 | NM_001127350 | Hs.479911 | ENSG00000130772 |
| MEFV | 4.22 | 3.28 | Mediterranean fever | 4210 | NM_000243 | Hs.632221 | ENSG00000103313 |
| METTL20 | 3.37 | 2.51 | methyltransferase like 20 | 254013 | NM_001135863 | Hs.740628 | ENSG00000139160 |
| METTL21A | 3.85 | 3.08 | methyltransferase like 21A | 151194 | NM_001127395 | Hs.664764 | ENSG00000144401 |
| METTL2A | 2.57 | 2.09 | methyltransferase like 2A | 339175 | NM_001005372 | Hs.381204 | ENSG00000087995 |
| METTL2B | 2.5 | 2.04 | methyltransferase like 2B | 55798 | NM_018396 | Hs.433213 | ENSG00000165055 |
| METTL8 | 3.18 | 2.57 | methyltransferase like 8 | 79828 | NM_024770 | Hs.135146 | ENSG00000123600 |
| MFAP5 | 4.32 | 4.07 | microfibrillar associated | 8076 | NM_001297709 | Hs.512842 | ENSG00000197614 |
| | | | protein 5 | | | | |
| MFSD11 | 2.35 | 2.09 | major facilitator superfamily | 79157 | NM_001242532 | Hs.73965 | ENSG00000092931 |
| | | | domain containing 11 | | | | |
| MGC27345 | 2.95 | 2.55 | uncharacterized protein | 157247 | NM_175880 | Hs.552129 | |
| | | | MGC27345 | | | | |
| MIRLET7BHG | 3.92 | 2.93 | MIRLET7B host gene | 400931 | NM_207477 | Hs.235838 | ENSG00000197182 |
| MLANA | 3.42 | 3.21 | melan-A | 2315 | NM_005511 | Hs.154069 | ENSG00000120215 |
| MMD2 | 4.97 | 4.03 | monocyte to macrophage | 221938 | NM_001100600 | Hs.558694 | ENSG00000136297 |
| | | | differentiation-associated 2 | | | | |
| MMS22L | 2.35 | 2.08 | MMS22-like, DNA repair | 253714 | NM_198468 | Hs.444292 | ENSG00000146263 |
| | | | protein | | | | |
| MOCS3 | 3.22 | 2.55 | molybdenum cofactor | 27304 | NM_014484 | Hs.159410 | ENSG00000124217 |
| | | | synthesis 3 | | | | |
| MOG | 4.36 | 3.37 | myelin oligodendrocyte | 4340 | NM_001008228 | Hs.141308 | ENSG00000204655 |
| | | | glycoprotein | | | | |
| MORN4 | 3.72 | 2.72 | MORN repeat containing 4 | 118812 | NM_001098831 | Hs.217409 | ENSG00000171160 |
| MPPE1 | 2.83 | 2.35 | metallophosphoesterase 1 | 65258 | NM_001242904 | Hs.712666 | ENSG00000154889 |
| MPV17L | 3.24 | 2.81 | MPV17 mitochondrial | 255027 | NM_001128423 | Hs.720673 | ENSG00000275543 |
| | | | membrane protein-like | | | | |
| MPZL3 | 2.69 | 2.15 | myelin protein zero-like 3 | 196264 | NM_001286152 | Hs.15396 | ENSG00000160588 |
| MREG | 3.18 | 2.62 | melanoregulin | 55686 | NM_018000 | Hs.620391 | ENSG00000118242 |
| MRGPRX3 | 4.79 | 3.53 | MAS-related GPR, member | 117195 | NM_054031 | Hs.380177 | ENSG00000179826 |
| | | | X3 | | | | |
| MS4A10 | 3.65 | 3 | membrane-spanning 4- | 341116 | NM_206893 | Hs.591956 | ENSG00000172689 |
| | | | domains, subfamily A, | | | | |
| | | | member 10 | | | | |
| MTFMT | 3.44 | 2.79 | mitochondrial methionyl- | 123263 | NM_139242 | Hs.531615 | ENSG00000103707 |
| | | | tRNA formyltransferase | | | | |
| MTG2 | 2.51 | 2.02 | mitochondrial ribosome- | 26164 | NM_015666 | Hs.340636 | ENSG00000101181 |
| | | | associated GTPase 2 | | | | |
| MTRNR2L5 | 6.94 | 5.47 | MT-RNR2-like 5 | 100463 289 | NM_001190478 | Hs.727204 | ENSG00000249860 |
| MXRA7 | 2.44 | 2.12 | matrix-remodeling | 439921 | NM_001008528 | Hs.250723 | ENSG00000182534 |
| | | | associated 7 | | | | |
| MYEOV2 | 0.48 | 0.51 | myeloma overexpressed 2 | 150678 | NM_001163424 | Hs.293884 | ENSG00000172428 |
| MYLK3 | 3.77 | 3.22 | myosin light chain kinase 3 | 91807 | NM_001308301 | Hs.130465 | ENSG00000140795 |
| NANOG | 4.75 | 3.1 | Nanog homeobox | 79923 | NM_001297698 | Hs.635882 | ENSG00000111704 |
| NCRUPAR | 4.14 | 3.71 | non-protein coding RNA, | 100302 746 | NR_028375 | | |
| | | | upstream of F2R/PAR1 | | | | |
| NEK2 | 4.2 | 3.18 | NIMA-related kinase 2 | 4751 | NM_001204182 | Hs.153704 | ENSG00000117650 |
| NEK8 | 2.71 | 2.3 | NIMA-related kinase 8 | 284086 | NM_178170 | Hs.448468 | ENSG00000160602 |
| NEXN-AS1 | 3.79 | 3.32 | NEXN antisense RNA 1 | 374987 | NM_001039463 | Hs.632414 | ENSG00000235927 |
| NLRP12 | 4.78 | 3.59 | NLR family, pyrin domain | 91662 | NM_001277126 | Hs.631573 | ENSG00000142405 |
| | | | containing 12 | | | | |
| NMNAT1 | 3.68 | 2.96 | nicotinamide nucleotide | 64802 | NM_001297778 | Hs.633762 | ENSG00000173614 |
| | | | adenylyltransferase 1 | | | | |
| NPFFR2 | 4.64 | 3.53 | neuropeptide FF receptor 2 | 10886 | NM_001144756 | Hs.99231 | ENSG00000056291 |
| NPHS1 | 3.6 | 3.16 | nephrosis 1, congenital, | 4868 | NM_004646 | Hs.122186 | ENSG00000161270 |
| | | | Finnish type (nephrin) | | | | |
| NQO1 | 3.2 | 2.27 | NAD(P)H dehydrogenase, | 1728 | NM_000903 | Hs.406515 | ENSG00000181019 |
| | | | quinone 1 | | | | |
| NRIP2 | 2.49 | 2.5 | nuclear receptor interacting | 83714 | NM_031474 | Hs.530816 | ENSG00000053702 |
| | | | protein 2 | | | | |
| NRIP3 | 3.99 | 2.93 | nuclear receptor interacting | 56675 | NM_020645 | Hs.523467 | ENSG00000175352 |
| | | | protein 3 | | | | |
| NT5DC3 | 3.57 | 2.85 | 5′-nucleotidase domain | 51559 | NM_001031701 | Hs.48428 | ENSG00000111696 |
| | | | containing 3 | | | | |
| NUBPL | 3.17 | 2.32 | nucleotide binding protein- | 80224 | NM_001201573 | Hs.288981 | ENSG00000151413 |
| | | | like | | | | |
| NUGGC | 2.57 | 2.48 | nuclear GTPase, germinal | 389643 | NM_001010906 | Hs.370129 | ENSG00000189233 |
| | | | center associated | | | | |
| NXN | 4.95 | 3.79 | nucleoredoxin | 64359 | NM_001205319 | Hs.527989 | ENSG00000167693 |
| NXNL2 | 4.52 | 3.62 | nucleoredoxin-like 2 | 158046 | NM_001161625 | Hs.734507 | ENSG00000130045 |
| NYAP2 | 3.86 | 3.11 | neuronal tyrosine- | 57624 | NM_020864 | Hs.224409 | ENSG00000144460 |
| | | | phosphorylated | | | | |
| | | | phosphoinositide-3-kinase | | | | |
| | | | adaptor 2 | | | | |
| OCLN | 2.79 | 2.4 | occludin | 100506 658 | NM_001205254 | Hs.592605 | ENSG00000197822 |
| ODF2L | 4.02 | 3.1 | outer dense fiber of sperm | 57489 | NM_001007022 | Hs.149360 | ENSG00000122417 |
| | | | tails 2-like | | | | |
| OLAH | 4.85 | 3.6 | oleoy1-ACP hydrolase | 55301 | NM_001039702 | Hs.24309 | ENSG00000152463 |
| OPHN1 | 4.66 | 3.31 | oligophrenin 1 | 4983 | NM_002547 | Hs.128824 | ENSG00000079482 |
| OR11A1 | 4.75 | 3.53 | olfactory receptor, family 11, | 26531 | NM_013937 | Hs.676010 | ENSG00000204694 |
| | | | subfamily A, member 1 | | | | |
| OR7D2 | 3.8 | 3.11 | olfactory receptor, family 7, | 162998 | NM_175883 | Hs.531755 | ENSG00000188000 |
| | | | subfamily D, member 2 | | | | |
| OR7E91P | 6.26 | 4.84 | olfactory receptor, family 7, | 79315 | NR_002185 | Hs.327033 | ENSG00000205847 |
| | | | subfamily E, member 91 | | | | |
| | | | pseudogene | | | | |
| ORAI2 | 3.08 | 2.65 | ORAI calcium release- | 80228 | NM_001126340 | Hs.363308 | ENSG00000160991 |
| | | | activated calcium modulator 2 | | | | |
| ORC4 | 4.42 | 3.38 | origin recognition complex, | 5000 | NM_001190879 | Hs.558364 | ENSG00000115947 |
| | | | subunit 4 | | | | |
| ORC6 | 3.75 | 3.32 | origin recognition complex, | 23594 | NM_014321 | Hs.49760 | ENSG00000091651 |
| | | | subunit 6 | | | | |
| OSBPL2 | 2.32 | 1.98 | oxysterol binding protein- | 9885 | NM_001001691 | Hs.473254 | ENSG00000130703 |
| | | | like 2 | | | | |
| OSGEPL1-AS1 | 3.23 | 2.45 | OSGEPL1 antisense RNA 1 | 101409 258 | NR_102429 | Hs.738558 | |
| OTUD6A | 5.09 | 4.14 | OTU deubiquitinase 6A | 139562 | NM_207320 | Hs.447381 | ENSG00000189401 |
| P2RX5-TAX1BP3 | 3.14 | 2.64 | P2RX5-TAX1BP3 | 100533 970 | NR_037928 | Hs.731607 | ENSG00000257950 |
| | | | readthrough (NMD | | | | |
| | | | candidate) | | | | |
| PABPC1P2 | 3.85 | 2.98 | poly(A) binding protein, | 728773 | NR_026904 | Hs.334462 | |
| | | | cytoplasmic 1 pseudogene 2 | | | | |
| PACS2 | 2.18 | 2.2 | phosphofurin acidic cluster | 23241 | NM_001100913 | Hs.525626 | ENSG00000179364 |
| | | | sorting protein 2 | | | | |
| PAQR7 | 3.26 | 2.65 | progestin and adipoQ | 164091 | NM_178422 | Hs.523652 | ENSG00000182749 |
| | | | receptor family member VII | | | | |
| PARD6G | 4.04 | 3.5 | par-6 family cell polarity | 84552 | NM_032510 | Hs.654920 | ENSG00000178184 |
| | | | regulator gamma | | | | |
| PARK2 | 3.51 | 3 | parkin RBR E3 ubiquitin | 5071 | NM_004562 | Hs.132954 | ENSG00000185345 |
| | | | protein ligase | | | | |
| PART1 | 4.74 | 3.77 | prostate androgen-regulated | 25859 | NM_001039499 | Hs.146312 | ENSG00000152931 |
| | | | transcript 1 (non-protein | | | | |
| | | | coding) | | | | |
| PAXBP1-AS1 | 4.11 | 3.3 | PAXBP1 antisense RNA 1 | 100506 215 | NR_038879 | Hs.657123 | ENSG00000238197 |
| PCAT18 | 4.34 | 3.61 | prostate cancer associated | 728606 | NR_024259 | Hs.170599 | ENSG00000265369 |
| | | | transcript 18 (non-protein | | | | |
| | | | coding) | | | | |
| PCBD2 | 2.76 | 2.39 | pterin-4 alpha-carbinolamine | 84105 | NM_032151 | Hs.710014 | ENSG00000132570 |
| | | | dehydratase/dimerization | | | | |
| | | | cofactor of hepatocyte | | | | |
| | | | nuclear factor 1 alpha | | | | |
| | | | (TCF1)2 | | | | |
| PCDH11X | 4.44 | 3.85 | protocadherin 11 X-linked | 27328 | NM_001168360 | Hs.655673 | ENSG00000102290 |
| PCDH11Y | 5.34 | 3.89 | protocadherin 11 Y-linked | 83259 | NM_001278619 | Hs.661308 | ENSG00000099715 |
| PCDHB9 | 4.21 | 3.51 | protocadherin beta 9 | 56127 | NM_0191-19 | Hs.662726 | ENSG00000177839 |
| PDDC1 | 3.44 | 2.9 | Parkinson disease 7 domain | 347862 | NM_182612 | Hs.218362 | ENSG00000177225 |
| | | | containing 1 | | | | |
| PDE4C | 4.64 | 3.82 | phosphodiesterase 4C, | 5143 | NM_000923 | Hs.132584 | ENSG00000105650 |
| | | | CAMP-specific | | | | |
| PDE6A | 4.29 | 3.55 | phosphodiesterase 6A, | 5145 | NM_000440 | Hs.567314 | ENSG00000132915 |
| | | | CGMP-specific, rod, alpha | | | | |
| PDLIM5 | 2.84 | 2.51 | PDZ and LIM domain 5 | 10611 | NM_001011513 | Hs.480311 | ENSG00000163110 |
| PDP2 | 3.15 | 2.47 | pyruvate dehyrogenase | 57546 | NM_020786 | Hs.632214 | ENSG00000172840 |
| | | | phosphatase catalytic subunit 2 | | | | |
| PEX13 | 2.2 | 1.9 | peroxisomal biogenesis | 5194 | NM_002618 | Hs.161377 | ENSG00000162928 |
| | | | factor 13 | | | | |
| PGAM5 | 2.87 | 2.34 | PGAM family member 5, | 192111 | NM_001170543 | Hs.102558 | ENSG00000247077 |
| | | | serine/threonine protein | | | | |
| | | | phosphatase, mitochondrial | | | | |
| PGM2L1 | 2.47 | 2.14 | phosphoglucomutase 2-like 1 | 283209 | NM_173582 | Hs.26612 | ENSG00000165434 |
| PGM5P2 | 4.87 | 3.76 | phosphoglucomutase 5 | 595135 | NR_00283 | Hs.571593 | ENSG00000277778 |
| | | | pseudogene 2 | | | | |
| PHACTR4 | 2.21 | 1.9 | phosphatase and actin | 65979 | NM_001048183 | Hs.225641 | ENSG00000204138 |
| | | | regulator 4 | | | | |
| PHAX | 2.1 | 1.93 | phosphorylated adaptor for | 51808 | NM_032177 | Hs.555731 | ENSG00000164902 |
| | | | RNA export | | | | |
| PHYHD1 | 4.22 | 3.21 | phytanoyl-CoA dioxygenase | 254295 | NM_001100876 | Hs.709447 | ENSG00000175287 |
| | | | domain containing 1 | | | | |
| PIGX | 2.62 | 2.3 | phosphatidylinositol glycan | 54965 | NM_001166304 | Hs.223296 | ENSG00000163964 |
| | | | anchor biosynthesis, class X | | | | |
| PIN4P1 | 3.8 | 3.03 | protein (peptidylprolyl | 728758 | NR_003571 | Hs.658099 | |
| | | | cis/trans isomerase) NIMA- | | | | |
| | | | interacting, 4 pseudogene 1 | | | | |
| PLCXD1 | 2.76 | 2.34 | phosphatidylinositol-specific | 55344 | NM_018390 | Hs.522568 | ENSG00000182378 |
| | | | phospholipase C, X domain | | | | |
| | | | containing 1 | | | | |
| PLEKHA5 | 3.35 | 2.56 | pleckstrin homology domain | 54477 | NM_001143821 | Hs.188614 | ENSG00000052126 |
| | | | containing, family A | | | | |
| | | | member 5 | | | | |
| PNMA2 | 3.78 | 2.97 | paraneoplastic Ma antigen 2 | 10687 | NM_007257 | Hs.591838 | ENSG00000240694 |
| PNPO | 3.15 | 2.5 | pyridoxamine 5′-phosphate | 55163 | NM_018129 | Hs.631742 | ENSG00000108439 |
| | | | oxidase | | | | |
| PNPT1 | 2.47 | 2.21 | polyribonucleotide | 87178 | NM_033109 | Hs.388733 | ENSG00000138035 |
| | | | nucleotidyltransferase 1 | | | | |
| POU2AF1 | 3.86 | 2.91 | POU class 2 associating | 5450 | NM_006235 | Hs.654525 | ENSG00000110777 |
| | | | factor 1 | | | | |
| POU5F1 | 4.39 | 3.66 | POU class 5 homeobox 1 | 5460 | NM_001173531 | Hs.249184 | ENSG00000204531 |
| PPARA | 2.01 | 1.91 | peroxisome proliferator- | 5465 | NM_001001928 | Hs.103110 | ENSG00000186951 |
| | | | activated receptor alpha | | | | |
| PPFIBP1 | 2.93 | 2.51 | PTPRF interacting protein, | 8496 | NM_001198915 | Hs.172445 | ENSG00000110841 |
| | | | binding protein 1 (liprin beta 1) | | | | |
| PPIEL | 3.32 | 2.92 | peptidylprolyl isomerase E- | 728448 | NR_003929 | Hs.472508 | |
| | | | like pseudogene | | | | |
| PPIL6 | 3.58 | 2.99 | peptidylprolyl isomerase | 285755 | NM_001111298 | Hs.32234 | ENSG00000185250 |
| | | | (cyclophilin)-like 6 | | | | |
| PPP1R3B | 3.14 | 2.43 | protein phosphatase 1, | 79660 | NM_001201329 | Hs.458513 | ENSG00000173281 |
| | | | regulatory subunit 3B | | | | |
| PQLC2 | 3.19 | 3.02 | PQ loop repeat containing 2 | 54896 | NM_001040125 | Hs.647620 | ENSG00000040487 |
| PRELID2 | 3.66 | 2.93 | PRELI domain containing 2 | 153768 | NM_138492 | Hs.314261 | ENSG00000186314 |
| PRICKLE2-AS3 | 5.03 | 3.97 | PRICKLE2 antisense RNA 3 | 100874 243 | NR_046702 | Hs.670840 | ENSG00000226017 |
| PRKAR2A-AS1 | 3.81 | 3.22 | PRKAR2A antisense RNA 1 | 100506 637 | NR_109996 | Hs.634259 | ENSG00000224424 |
| PRNCR1 | 3.97 | 3.27 | prostate cancer associated | 101867 536 | NR_109833 | Hs.652970 | ENSG00000282961 |
| | | | non-coding RNA 1 | | | | |
| PRR11 | 3.89 | 3.18 | proline rich 11 | 55771 | NM_018304 | Hs.631750 | ENSG00000068489 |
| PRR7-AS1 | 2.95 | 2.56 | PRR7 antisense RNA 1 | 340037 | NR_038915 | Hs.570879 | |
| PSPH | 2.58 | 1.85 | phosphoserine phosphatase | 5723 | NM_004577 | Hs.512656 | ENSG00000146733 |
| PSTPIP2 | 3.42 | 2.89 | proline-serine-threonine | 9050 | NM_024430 | Hs.567384 | ENSG00000152229 |
| | | | phosphatase interacting | | | | |
| | | | protein 2 | | | | |
| PTCHD4 | 5.32 | 4.04 | patched domain containing 4 | 442213 | NM_001013732 | Hs.659409 | ENSG00000244694 |
| PTCSC3 | 4.39 | 3.47 | papillary thyroid carcinoma | 100886 964 | NR_049735 | Hs.742592 | |
| | | | susceptibility candidate 3 | | | | |
| | | | (non-protein coding) | | | | |
| PTGER4P2- | 5.14 | 3.65 | PTGER4P2-CDK2AP2P2 | 442421 | NR_024496 | Hs.585349 | |
| CDK2AP2P2 | | | readthrough transcribed | | | | |
| | | | pseudogene | | | | |
| PTGES2-AS1 | 2.85 | 2.88 | PTGES2 antisense RNA 1 | 389791 | NM_001013652 | Hs.632678 | ENSG00000232850 |
| | | | (head to head) | | | | |
| PTK6 | 3.01 | 2.83 | protein tyrosine kinase 6 | 5753 | NM_001256358 | Hs.51133 | ENSG00000101213 |
| PTOV1-AS1 | 2.32 | 2.25 | PTOV1 antisense RNA 1 | 100506 033 | NR_040037 | Hs.654814 | ENSG00000268006 |
| PTPRG-AS1 | 4.5 | 3.35 | PTPRG antisense RNA 1 | 100506 994 | NR_038281 | Hs.656620 | ENSG00000241472 |
| PXMP4 | 3.14 | 2.44 | peroxisomal membrane | 11264 | NM_007238 | Hs.654857 | ENSG00000101417 |
| | | | protein 4, 24 kDa | | | | |
| QPCTL | 3.58 | 3.17 | glutaminyl-peptide | 54814 | NM_001163377 | Hs.631556 | ENSG00000011478 |
| | | | cyclotransferase-like | | | | |
| QPRT | 2.91 | 2.92 | quinolinate | 23475 | NM_014298 | Hs.513484 | ENSG00000103485 |
| | | | phosphoribosyltransferase | | | | |
| RAB36 | 3.79 | 3.16 | RAB36, member RAS | 9609 | NM_004914 | Hs.369557 | ENSG00000100228 |
| | | | oncogene family | | | | |
| RAB42 | 4.45 | 3.72 | RAB42, member RAS | 115273 | NM_001193532 | Hs.652321 | ENSG00000188060 |
| | | | oncogene family | | | | |
| RAMP2-AS1 | 5.2 | 3.97 | RAMP2 antisense RNA 1 | 100190 938 | NR_024461 | Hs.655265 | ENSG00000197291 |
| RASAL2-AS1 | 4.08 | 3.27 | RASAL2 antisense RNA 1 | 100302 401 | NR_027982 | Hs.736117 | ENSG00000224687 |
| RBBP5 | 2.29 | 1.81 | retinoblastoma binding | 5929 | NM_001193272 | Hs.519230 | ENSG00000117222 |
| | | | protein 5 | | | | |
| RBBP9 | 2.52 | 2 | retinoblastoma binding | 10741 | NM_006606 | Hs.69330 | ENSG00000089050 |
| | | | protein 9 | | | | |
| RBM34 | 2.81 | 2.35 | RNA binding motif protein | 23029 | NM_001161533 | Hs.535224 | ENSG00000188739 |
| | | | 34 | | | | |
| RBMS2 | 3.36 | 2.82 | RNA binding motif, single | 5939 | NM_002898 | Hs.505729 | ENSG00000076067 |
| | | | stranded interacting protein 2 | | | | |
| RDH10 | 2.61 | 2.36 | retinol dehydrogenase 10 | 157506 | NM_172037 | Hs.244940 | ENSG00000121039 |
| | | | (all-trans) | | | | |
| RFT1 | 2.28 | 2.1 | RFT1 homolog | 91869 | NM_052859 | Hs.631910 | ENSG00000163933 |
| RHBG | 3.7 | 3.21 | Rh family, B glycoprotein | 57127 | NM_001256395 | Hs.131835 | ENSG00000132677 |
| | | | (gene/pseudogene) | | | | |
| RHD | 2.91 | 2.68 | Rh blood group, D antigen | 6007 | NM_001127691 | Hs.449968 | ENSG00000187010 |
| RIPPLY3 | 4.26 | 3.28 | ripply transcriptional | 53820 | NM_018962 | Hs.254560 | ENSG00000183145 |
| | | | repressor 3 | | | | |
| RNF144A-AS1 | 4.07 | 2.8 | RNF144A antisense RNA 1 | 386597 | NR_033997 | Hs.559010 | ENSG00000228203 |
| RNF207 | 3.76 | 2.95 | ring finger protein 207 | 388591 | NM_173795 | Hs.716549 | ENSG00000158286 |
| RNF222 | 3.81 | 3.32 | ring finger protein 222 | 643904 | NM_001146684 | Hs.526550 | ENSG00000189051 |
| ROR1-AS1 | 4.17 | 3.15 | ROR1 antisense RNA 1 | 101927 034 | NR_110665 | Hs.680824 | ENSG00000223949 |
| RPL23AP53 | 3.22 | 2.57 | ribosomal protein L23a | 644128 | NR_003572 | Hs.652159 | |
| | | | pseudogene 53 | | | | |
| RUNDC1 | 3.11 | 2.63 | RUN domain containing 1 | 146923 | NM_173079 | Hs.632255 | ENSG00000198863 |
| S1PR2 | 3.38 | 2.86 | sphingosine-1-phosphate | 9294 | NM_004230 | Hs.655405 | ENSG00000267534 |
| | | | receptor 2 | | | | |
| SAA2 | 4.24 | 3.13 | serum amyloid A2 | 6289 | NM_001127380 | Hs.731376 | ENSG00000134339 |
| SCAI | 2.63 | 2.39 | suppressor of cancer cell | 286205 | NM_001144877 | Hs.59504 | ENSG00000173611 |
| | | | invasion | | | | |
| SCD5 | 4.02 | 3.09 | stearoyl-CoA desaturase 5 | 79966 | NM_001037582 | Hs.379191 | ENSG00000145284 |
| SCHLAP1 | 4.03 | 3.25 | SWI/SNF complex | 101669 767 | NR_104319 | | ENSG00000281131 |
| | | | antagonist associated with | | | | |
| | | | prostate cancer 1 (non- | | | | |
| | | | protein coding) | | | | |
| SEC14L4 | 4.22 | 3.23 | SEC14-like lipid binding 4 | 284904 | NM_001161368 | Hs.517541 | ENSG00000133488 |
| SEC24B-AS1 | 3.14 | 2.68 | SEC24B antisense RNA 1 | 100533 182 | NR_039978 | Hs.518927 | ENSG00000247950 |
| SEPSECS-AS1 | 2.6 | 4.21 | SEPSECS antisense RNA 1 | 285540 | NR_037934 | Hs.732278 | |
| | | | (head to head) | | | | |
| SFTPB | 3.95 | 3.17 | surfactant protein B | 6439 | NM_000542 | Hs.512690 | ENSG00000168878 |
| SGCB | 2.27 | 1.97 | sarcoglycan, beta (43 kDa | 6443 | NM_000232 | Hs.438953 | ENSG00000163069 |
| | | | dystrophin-associated | | | | |
| | | | glycoprotein) | | | | |
| SGOL1 | 3.16 | 2.77 | shugoshin-like 1 (S. pombe) | 151648 | NM_001012409 | Hs.105153 | ENSG00000129810 |
| SGSM1 | 3.88 | 3.15 | small G protein signaling | 129049 | NM_001039948 | Hs.474397 | ENSG00000167037 |
| | | | modulator 1 | | | | |
| SHANK2-AS3 | 4.12 | 3.3 | SHANK2 antisense RNA 3 | 220070 | NM_145308 | Hs.326766 | ENSG00000171671 |
| SHISA9 | 5.02 | 3.8 | shisa family member 9 | 729993 | NM_001145204 | Hs.130661 | ENSG00000237515 |
| SHOX | 2.82 | 2.39 | short stature homeobox | 6473 | NM_000451 | Hs.105932 | ENSG00000185960 |
| SHROOM1 | 4.92 | 3.67 | shroom family member 1 | 134549 | NM_001172700 | Hs.519574 | ENSG00000164403 |
| SIGLEC10 | 3.86 | 2.8 | sialic acid binding Ig-like | 89790 | NM_001171156 | Hs.284813 | ENSG00000142512 |
| | | | lectin 10 | | | | |
| SIRPB2 | 3.31 | 2.78 | signal-regulatory protein | 284759 | NM_001122962 | Hs.721685 | ENSG00000196209 |
| | | | beta 2 | | | | |
| SIX4 | 4.13 | 3.33 | SIX homeobox 4 | 51804 | NM_017420 | Hs.97849 | ENSG00000100625 |
| SKA1 | 4.3 | 3.38 | spindle and kinetochore | 220134 | NM_001039535 | Hs.134726 | ENSG00000154839 |
| | | | associated complex subunit 1 | | | | |
| SKP2 | 2.69 | 2.14 | S-phase kinase-associated | 6502 | NM_001243120 | Hs.23348 | ENSG00000145604 |
| | | | protein 2, E3 ubiquitin | | | | |
| | | | protein ligase | | | | |
| SLC14A2 | 4.34 | 3.33 | solute carrier family 14 (urea | 8170 | NM_001242692 | Hs.710927 | ENSG00000132874 |
| | | | transporter), member 2 | | | | |
| SLC15A1 | 3.52 | 2.91 | solute carrier family 15 | 6564 | NM_005073 | Hs.436893 | ENSG00000088386 |
| | | | (oligopeptide transporter), | | | | |
| | | | member 1 | | | | |
| SLC16A4 | 3.6 | 2.98 | solute carrier family 16, | 9122 | NM_001201546 | Hs.351306 | ENSG00000168679 |
| | | | member 4 | | | | |
| SLC25A15 | 3.84 | 3.09 | solute carrier family 25 | 10166 | NM_014252 | Hs.646645 | ENSG00000102743 |
| | | | (mitochondrial carrier; | | | | |
| | | | ornithine transporter) | | | | |
| | | | member 15 | | | | |
| SLC28A2 | 4.35 | 3.53 | solute carrier family 28 | 9153 | NM_004212 | Hs.367833 | ENSG00000137860 |
| | | | (concentrative nucleoside | | | | |
| | | | transporter), member 2 | | | | |
| SLC31A1 | 4.06 | 3.11 | solute carrier family 31 | 1317 | NM_001859 | Hs.532315 | ENSG00000136868 |
| | | | (copper transporter), member 1 | | | | |
| SLC35E3 | 2.91 | 2.43 | solute carrier family 35, | 55508 | NM_018656 | Hs.506011 | ENSG00000175782 |
| | | | member E3 | | | | |
| SLC36A2 | 4.11 | 3.19 | solute carrier family 36 | 153201 | NM_181776 | Hs.483877 | ENSG00000186335 |
| | | | (proton/amino acid | | | | |
| | | | symporter), member 2 | | | | |
| SLC37A2 | 4.9 | 3.85 | solute carrier family 37 | 219855 | NM_001145290 | Hs.352661 | ENSG00000134955 |
| | | | (glucose-6-phosphate | | | | |
| | | | transporter), member 2 | | | | |
| SLC44A4 | 5.05 | 3.52 | solute carrier family 44, | 80736 | NM_001178044 | Hs.335355 | ENSG00000204385 |
| | | | member 4 | | | | |
| SLC4A1 | 3.33 | 2.73 | solute carrier family 4 (anion | 6521 | NM_000342 | Hs.210751 | ENSG00000004939 |
| | | | exchanger), member 1 | | | | |
| | | | (Diego blood group) | | | | |
| SLC4A8 | 3.56 | 2.94 | solute carrier family 4, | 9498 | NM_001039960 | Hs.4749 | ENSG00000050438 |
| | | | sodium bicarbonate | | | | |
| | | | cotransporter, member 8 | | | | |
| SLC50A1 | 2.28 | 1.92 | solute carrier family 50 | 55974 | NM_001122837 | Hs.292154 | ENSG00000169241 |
| | | | (sugar efflux transporter), | | | | |
| | | | member 1 | | | | |
| SLC5A5 | 3.63 | 3.05 | solute carrier family 5 | 6528 | NM_000453 | Hs.584804 | ENSG00000105641 |
| | | | (sodium/iodide | | | | |
| | | | cotransporter), member 5 | | | | |
| SLC6A4 | 3.93 | 3.47 | solute carrier family 6 | 6532 | NM_001045 | Hs.29792 | ENSG00000108576 |
| | | | (neurotransmitter | | | | |
| | | | transporter), member 4 | | | | |
| SLC7A5P2 | 2.63 | 2.57 | solute carrier family 7 | 387254 | NR_002594 | Hs.448808 | |
| | | | (amino acid transporter light | | | | |
| | | | chain, L system), member 5 | | | | |
| | | | pseudogene 2 | | | | |
| SLC9A4 | 5.08 | 3.5 | solute carrier family 9, | 389015 | NM_001011552 | Hs.447686 | ENSG00000180251 |
| | | | subfamily A (NHE4, cation | | | | |
| | | | proton antiporter 4), member 4 | | | | |
| SLFNL1-AS1 | 3.57 | 2.93 | SLFNL1 antisense RNA 1 | 100507 178 | NR_037868 | Hs.660056 | ENSG00000281207 |
| SMG1P7 | 3.5 | 3.15 | SMG1 pseudogene 7 | 100506 060 | NR_033959 | Hs.655258 | ENSG00000261556 |
| SMIM14 | 3.45 | 2.75 | small integral membrane | 201895 | NM_174921 | Hs.205952 | ENSG00000163683 |
| | | | protein 14 | | | | |
| SMIM17 | 5.46 | 3.85 | small integral membrane | 147670 | NM_001193628 | Hs.336588 | ENSG00000268182 |
| | | | protein 17 | | | | |
| SNHG20 | 3.48 | 3.1 | small nucleolar RNA host | 654434 | NR_027058 | Hs.720923 | ENSG00000234912 |
| | | | gene 20 | | | | |
| SNHG4 | 4.17 | 3.63 | small nucleolar RNA host | 724102 | NR_003141 | Hs.268939 | |
| | | | gene 4 | | | | |
| SNX22 | 2.51 | 2.18 | sorting nexin 22 | 79856 | NM_024798 | Hs.744250 | ENSG00000157734 |
| SOX9-AS1 | 5.1 | 3.32 | SOX9 antisense RNA 1 | 400618 | NR_103737 | Hs.657374 | ENSG00000234899 |
| SPATS2 | 2.59 | 2.32 | spermatogenesis associated, | 65244 | NM_001293285 | Hs.654826 | ENSG00000123352 |
| | | | serine-rich 2 | | | | |
| SPATS2L | 2.78 | 2.29 | spermatogenesis associated, | 26010 | NM_001100422 | Hs.120323 | ENSG00000196141 |
| | | | serine-rich 2-like | | | | |
| SPC25 | 4.52 | 3.46 | SPC25, NDC80 kinetochore | 57405 | NM_020675 | Hs.421956 | ENSG00000152253 |
| | | | complex component | | | | |
| SPDYE8P | 2.11 | 1.98 | speedy/RINGO cell cycle | 728524 | NM_001023562 | Hs.571275 | |
| | | | regulator family member E8, | | | | |
| | | | pseudogene | | | | |
| SPIB | 3.67 | 2.75 | Spi-B transcription factor | 6689 | NM_001243998 | Hs.437905 | ENSG00000269404 |
| | | | (Spi-1/PU.1 related) | | | | |
| SPRED1 | 4.19 | 3.24 | sprouty-related, EVH1 | 161742 | NM_152594 | Hs.525781 | ENSG00000166068 |
| | | | domain containing 1 | | | | |
| SRRM2-AS1 | 3.86 | 3.24 | SRRM2 antisense RNA 1 | 100128 788 | NR_027274 | Hs.311208 | ENSG00000205913 |
| SRSF12 | 3.63 | 3.27 | serine/arginine-rich splicing | 135295 | NM_080743 | Hs.254414 | ENSG00000154548 |
| | | | factor 12 | | | | |
| STAC2 | 4.17 | 3.11 | SH3 and cysteine rich | 342667 | NM_198993 | Hs.145068 | ENSG00000141750 |
| | | | domain 2 | | | | |
| STAP2 | 3.25 | 2.98 | signal transducing adaptor | 55620 | NM_001013841 | Hs.194385 | ENSG00000178078 |
| | | | family member 2 | | | | |
| STAR | 3.6 | 2.66 | steroidogenic acute | 6770 | NM_000349 | Hs.521535 | ENSG00000147465 |
| | | | regulatory protein | | | | |
| STAU2-AS1 | 4.02 | 4.08 | STAU2 antisense RNA 1 | 100128 126 | NR_038406 | Hs.679921 | ENSG00000253302 |
| STRIP2 | 3.61 | 3.14 | striatin interacting protein 2 | 57464 | NM_001134336 | Hs.489988 | ENSG00000128578 |
| SWSAP1 | 2.79 | 2.31 | SWIM-type zinc finger 7 | 126074 | NM_175871 | Hs.631619 | ENSG00000173928 |
| | | | associated protein 1 | | | | |
| TAF8 | 2.86 | 2.4 | TAF8 RNA polymerase II, | 129685 | NM_138572 | Hs.520122 | ENSG00000137413 |
| | | | TATA box binding protein | | | | |
| | | | (TBP)-associated factor, | | | | |
| | | | 43 kDa | | | | |
| TANGO2 | 2.65 | 2.04 | transport and golgi | 128989 | NM_001283106 | Hs.474233 | ENSG00000183597 |
| | | | organization 2 homolog | | | | |
| TARS2 | 2.64 | 2.13 | threony1-tRNA synthetase 2, | 80222 | NM_001271895 | Hs.288974 | ENSG00000143374 |
| | | | mitochondrial (putative) | | | | |
| TATDN3 | 3.33 | 2.85 | TatD DNase domain | 128387 | NM_001042552 | Hs.530538 | ENSG00000203705 |
| | | | containing 3 | | | | |
| TBCID24 | 2.97 | 2.82 | TBC1 domain family, | 57465 | NM_001199107 | Hs.353087 | ENSG00000162065 |
| | | | member 24 | | | | |
| TBCCD1 | 2.64 | 2.29 | TBCC domain containing 1 | 55171 | NM_001134415 | Hs.518469 | ENSG00000113838 |
| TBXA2R | 3.83 | 3.16 | thromboxane A2 receptor | 6915 | NM_001060 | Hs.442530 | ENSG00000006638 |
| TEX101 | 3.65 | 3.17 | testis expressed 101 | 83639 | NM_001130011 | Hs.97978 | ENSG00000131126 |
| TFDP2 | 2.02 | 1.98 | transcription factor Dp-2 | 7029 | NM_001178138 | Hs.379018 | ENSG00000114126 |
| | | | (E2F dimerization partner 2) | | | | |
| THRIL | 3.15 | 2.63 | TNF and HNRNPL related | 102659 353 | NR_110375 | Hs.596464 | ENSG00000280634 |
| | | | immunoregulatory long non- | | | | |
| | | | coding RNA | | | | |
| TIGD1 | 2.33 | 2.38 | tigger transposable element | 200765 | NM_145702 | Hs.211823 | ENSG00000221944 |
| | | | derived 1 | | | | |
| TINCR | 2.55 | 2.42 | tissue differentiation- | 257000 | NM_153375 | Hs.515575 | ENSG00000223573 |
| | | | inducing non-protein coding | | | | |
| | | | RNA | | | | |
| TLCD2 | 4.68 | 3.6 | TLC domain containing 2 | 727910 | NM_001164407 | Hs.531005 | ENSG00000185561 |
| TLR10 | 3.68 | 2.57 | toll-like receptor 10 | 81793 | NM_001017388 | Hs.120551 | ENSG00000174123 |
| TLR8-AS1 | 5.69 | 3.95 | TLR8 antisense RNA 1 | 349408 | NR_030727 | Hs.685035 | ENSG00000233338 |
| TMCC1-AS1 | 4.46 | 3.15 | TMCC1 antisense RNA 1 | 100507 032 | NR_037893 | Hs.529562 | ENSG00000271270 |
| | | | (head to head) | | | | |
| TMEM106A | 3.32 | 2.82 | transmembrane protein 106A | 113277 | NM_001291586 | Hs.536474 | ENSG00000184988 |
| TMEM120B | 2.62 | 2.28 | transmembrane protein 120B | 144404 | NM_001080825 | Hs.644504 | ENSG00000188735 |
| TMEM168 | 2.41 | 2.01 | transmembrane protein 168 | 64418 | NM_001287497 | Hs.606345 | ENSG00000146802 |
| TMEM212 | 4.48 | 3.34 | transmembrane protein 212 | 389177 | NM_001164436 | Hs.642307 | ENSG00000186329 |
| TMEM213 | 3.63 | 3.05 | transmembrane protein 213 | 155006 | NM_001085429 | Hs.567729 | ENSG00000214128 |
| TMEM236 | 3.74 | 3.34 | transmembrane protein 236 | 653567 | NM_001013629 | Hs.564139 | ENSG00000148483 |
| TMEM254-AS1 | 3.57 | 2.82 | TMEM254 antisense RNA 1 | 219347 | NR_027428 | Hs.524453 | |
| TMEM38A | 3.65 | 3.13 | transmembrane protein 38A | 79041 | NM_024074 | Hs.436068 | ENSG00000072954 |
| TMEM41B | 2.92 | 2.24 | transmembrane protein 41B | 440026 | NM_001165030 | Hs.594563 | ENSG00000166471 |
| TMIGD2 | 2.08 | 1.98 | transmembrane and | 126259 | NM_001169126 | Hs.263928 | ENSG00000167664 |
| | | | immunoglobulin domain | | | | |
| | | | containing 2 | | | | |
| TNFAIP8L1 | 3.03 | 2.55 | tumor necrosis factor, alpha- | 126282 | NM_001167942 | Hs.465643 | ENSG00000185361 |
| | | | induced protein 8-like 1 | | | | |
| TNFAIP8L2- | 6.07 | 4.08 | TNFAIP8L2-SCNM1 | 100534 012 | NM_001204848 | Hs.732060 | ENSG00000163156 |
| SCNM1 | | | readthrough | | | | |
| TONSL | 2.99 | 2.48 | tonsoku-like, DNA repair | 4796 | NM_01343. | Hs.675285 | ENSG00000160949 |
| | | | protein | | | | |
| TOR1AIP2 | 2.24 | 1.91 | torsin A interacting protein 2 | 163590 | NM_001199260 | Hs.571797 | ENSG00000169905 |
| TOR4A | 3.61 | 2.93 | torsin family 4, member A | 54863 | NM_017723 | Hs.495541 | ENSG00000198113 |
| TPMT | 2.9 | 2.63 | thiopurine S- | 7172 | NM_000367 | Hs.444319 | ENSG00000137364 |
| | | | methyltransferase | | | | |
| TPTEP1 | 3.95 | 2.81 | transmembrane phosphatase | 387590 | NR_001591 | Hs.474116 | ENSG00000100181 |
| | | | with tensin homology | | | | |
| | | | pseudogene 1 | | | | |
| TRAF3IP2 | 3.47 | 2.98 | TRAF3 interacting protein 2 | 10758 | NM_001164281 | Hs.561514 | ENSG00000056972 |
| TRAPPC2 | 2.11 | 2.01 | trafficking protein particle | 6399 | NM_001011658 | Hs.592238 | ENSG00000196459 |
| | | | complex 2 | | | | |
| TRIM16 | 2.7 | 2.5 | tripartite motif containing 16 | 10626 | NM_006470 | Hs.123534 | ENSG00000221926 |
| TRIM45 | 4.23 | 3.39 | tripartite motif containing 45 | 80263 | NM_001145635 | Hs.301526 | ENSG00000134253 |
| TRPV1 | 3.44 | 3.24 | transient receptor potential | 7442 | NM_018727 | Hs.579217 | ENSG00000196689 |
| | | | cation channel, subfamily V, | | | | |
| | | | member 1 | | | | |
| TSG1 | 4.82 | 3.93 | tumor suppressor TSG 1 | 643432 | NR_015362 | Hs.509936 | |
| TSIX | 4.23 | 3.42 | TSIX transcript, XIST | 9383 | NR_003255 | Hs.529901 | ENSG00000270641 |
| | | | antisense RNA | | | | |
| TSTD3 | 3.37 | 3.14 | thiosulfate sulfurtransferase | 100130 890 | NM_001195131 | Hs.634506 | ENSG00000228439 |
| | | | (rhodanese)-like domain | | | | |
| | | | containing 3 | | | | |
| TUBA3FP | 3.9 | 3.23 | tubulin, alpha 3f, pseudogene | 113691 | NR_003608 | Hs.585006 | ENSG00000161149 |
| TUFT1 | 3.19 | 2.99 | tuftelin 1 | 7286 | NM_001126337 | Hs.489922 | ENSG00000143367 |
| TVP23C | 2.66 | 2.56 | trans-golgi network vesicle | 201158 | NM_001135036 | Hs.164595 | ENSG00000175106 |
| | | | protein 23 homolog C (S. | | | | |
| | | | cerevisiae) | | | | |
| UBE2Q2P1 | 3.73 | 3.08 | ubiquitin-conjugating | 388165 | NM_207382 | Hs.498348 | ENSG00000189136 |
| | | | enzyme E2Q family member | | | | |
| | | | 2 pseudogene 1 | | | | |
| UBL7-AS1 | 4.09 | 3.42 | UBL7 antisense RNA 1 | 440288 | NR_038448 | Hs.611046 | ENSG00000247240 |
| | | | (head to head) | | | | |
| UBOX5 | 2.27 | 2.05 | U-box domain containing 5 | 22888 | NM_001267584 | Hs.654646 | ENSG00000185019 |
| UCKL1-AS1 | 3.97 | 3.53 | UCKL1 antisense RNA 1 | 100113 386 | NR_027287 | Hs.551552 | |
| UGDH-AS1 | 4.44 | 3.36 | UGDH antisense RNA 1 | 100885 776 | NR_047679 | Hs.640769 | ENSG00000249348 |
| UGGT1 | 2.1 | 1.94 | UDP-glucose glycoprotein | 56886 | NM_001025777 | Hs.743306 | ENSG00000136731 |
| | | | glucosyltransferase 1 | | | | |
| UGT8 | 4.93 | 3.72 | UDP glycosyltransferase 8 | 7368 | NM_001128174 | Hs.144197 | ENSG00000174607 |
| UPK1B | 4.09 | 3.31 | uroplakin 1B | 7348 | NM_006952 | Hs.271580 | ENSG00000114638 |
| USP49 | 2.46 | 2.25 | ubiquitin specific peptidase | 25862 | NM_001286554 | Hs.593575 | ENSG00000164663 |
| | | | 49 | | | | |
| USP54 | 2.37 | 2.16 | ubiquitin specific peptidase | 159195 | NM_152586 | Hs.657355 | ENSG00000166348 |
| | | | 54 | | | | |
| UTP11L | 3.22 | 2.35 | UTP11-like, U3 small | 51118 | NM_016037 | Hs.472038 | ENSG00000183520 |
| | | | nucleolar ribonucleoprotein | | | | |
| | | | (yeast) | | | | |
| UTS2B | 4.79 | 3.78 | urotensin 2B | 257313 | NM_198152 | Hs.518492 | ENSG00000188958 |
| VSIG1 | 2.55 | 2.09 | V-set and immunoglobulin | 340547 | NM_001170553 | Hs.177164 | ENSG00000101842 |
| | | | domain containing 1 | | | | |
| VSTM4 | 4.19 | 3.25 | V-set and transmembrane | 196740 | NM_001031746 | Hs.522928 | ENSG00000165633 |
| | | | domain containing 4 | | | | |
| WDR11-AS1 | 4.3 | 3.3 | WDR11 antisense RNA 1 | 283089 | NR_033850 | Hs.568750 | ENSG00000227165 |
| WDR45 | 2.27 | 1.9 | WD repeat domain 45 | 11152 | NM_001029896 | Hs.632807 | ENSG00000196998 |
| WDR92 | 2.37 | 1.67 | WD repeat domain 92 | 116143 | NM_001256476 | Hs.631877 | ENSG00000243667 |
| WFDC8 | 4.12 | 3.11 | WAP four-disulfide core | 90199 | NM_130896 | Hs.116128 | ENSG00000158901 |
| | | | domain 8 | | | | |
| WNT7B | 3.91 | 3.4 | wingless-type MMTV | 7477 | NM_058238 | Hs.512714 | ENSG00000188064 |
| | | | integration site family, | | | | |
| | | | member 7B | | | | |
| XIAP | 2.32 | 1.93 | X-linked inhibitor of | 331 | NM_001157 | Hs.356076 | ENSG00000101966 |
| | | | apoptosis, E3 ubiquitin | | | | |
| | | | protein ligase | | | | |
| XKR9 | 4.97 | 3.6 | XK, Kell blood group | 389668 | NM_001011720 | Hs.458938 | ENSG00000221947 |
| | | | complex subunit-related | | | | |
| | | | family, member 9 | | | | |
| XPNPEP3 | 2.73 | 2.35 | X-prolyl aminopeptidase 3, | 63929 | NM_001204827 | Hs.529163 | ENSG00000196236 |
| | | | mitochondrial | | | | |
| XRCC2 | 3.95 | 3.39 | X-ray repair complementing | 7516 | NM_0054 | Hs.647093 | ENSG00000196584 |
| | | | defective repair in Chinese | | | | |
| | | | hamster cells 2 | | | | |
| ZBTB8A | 3.97 | 3.28 | zinc finger and BTB domain | 653121 | NM_001040441 | Hs.546479 | ENSG00000160062 |
| | | | containing 8A | | | | |
| ZC3H12D | 2.26 | 2.3 | zinc finger CCCH-type | 340152 | NM_207360 | Hs.632618 | ENSG00000178199 |
| | | | containing 12D | | | | |
| ZFP14 | 2.62 | 1.96 | ZFP14 zinc finger protein | 57677 | NM_001297619 | Hs.35524 | ENSG00000142065 |
| ZFP30 | 2.66 | 2.28 | ZFP30 zinc finger protein | 22835 | NM_014898 | Hs.716719 | ENSG00000120784 |
| ZFP42 | 3.79 | 2.91 | ZFP42 zinc finger protein | 132625 | NM_001304358 | Hs.335787 | ENSG00000179059 |
| ZKSCAN3 | 3.87 | 2.77 | zinc finger with KRAB and | 80317 | NM_001242894 | Hs.380930 | ENSG00000189298 |
| | | | SCAN domains 3 | | | | |
| ZKSCAN7 | 2.64 | 2.17 | zinc finger with KRAB and | 55888 | NM_001288590 | Hs.529512 | ENSG00000196345 |
| | | | SCAN domains 7 | | | | |
| ZMYM5 | 2.23 | 1.95 | zinc finger, MYM-type 5 | 9205 | NM_001039649 | Hs.530988 | ENSG00000132950 |
| ZNF154 | 2.49 | 2.26 | zinc finger protein 154 | 7710 | NM_001085384 | Hs.646378 | ENSG00000179909 |
| ZNF2 | 3.33 | 2.35 | zinc finger protein 2 | 7549 | NM_001017396 | Hs.590916 | ENSG00000275111 |
| ZNF264 | 2.16 | 1.84 | zinc finger protein 264 | 9422 | NM_003417 | Hs.515634 | ENSG00000083844 |
| ZNF286B | 3.02 | 2.56 | zinc finger protein 286B | 729288 | NM_001145045 | Hs.534279 | ENSG00000249459 |
| ZNF34 | 4 | 3.19 | zinc finger protein 34 | 80778 | NM_001286769 | Hs.631854 | ENSG00000196378 |
| ZNF347 | 3.18 | 2.72 | zinc finger protein 347 | 84671 | NM_001172674 | Hs.467239 | ENSG00000197937 |
| ZNF471 | 5.04 | 3.57 | zinc finger protein 471 | 57573 | NM_020813 | Hs.710590 | ENSG00000196263 |
| ZNF483 | 3.18 | 2.91 | zinc finger protein 483 | 158399 | NM_001007169 | Hs.584864 | ENSG00000173258 |
| ZNF490 | 2.8 | 2.42 | zinc finger protein 490 | 57474 | NM_020714 | Hs.655860 | ENSG00000188033 |
| ZNF492 | 3.5 | 2.92 | zinc finger protein 492 | 57615 | NM_020855 | Hs.232108 | ENSG00000229676 |
| ZNF526 | 3.18 | 2.4 | zinc finger protein 526 | 116115 | NM_133444 | Hs.137282 | ENSG00000167625 |
| ZNF527 | 2.89 | 2.38 | zinc finger protein 527 | 84503 | NM_032453 | Hs.590940 | ENSG00000189164 |
| ZNF543 | 2.48 | 2.13 | zinc finger protein 543 | 125919 | NM_213598 | Hs.202544 | ENSG00000178229 |
| ZNF554 | 3.36 | 2.65 | zinc finger protein 554 | 115196 | NM_001102651 | Hs.307043 | ENSG00000172006 |
| ZNF556 | 4.28 | 4.37 | zinc finger protein 556 | 80032 | NM_001300843 | Hs.287433 | ENSG00000172000 |
| ZNF562 | 2.57 | 2.1 | zinc finger protein 562 | 54811 | NM_001130031 | Hs.371107 | ENSG00000171466 |
| ZNF662 | 3.76 | 2.78 | zinc finger protein 662 | 389114 | NM_001134656 | Hs.720173 | ENSG00000182983 |
| ZNF665 | 4.04 | 3.18 | zinc finger protein 665 | 79788 | NM_024733 | Hs.745230 | ENSG00000197497 |
| ZNF677 | 3.48 | 2.9 | zinc finger protein 677 | 342926 | NM_182609 | Hs.20506 | ENSG00000197928 |
| ZNF713 | 3.93 | 3.69 | zinc finger protein 713 | 349075 | NM_182633 | Hs.660834 | ENSG00000178665 |
| ZNF716 | 3.7 | 3.18 | zinc finger protein 716 | 441234 | NM_001159279 | Hs.533121 | ENSG00000182111 |
| ZNF761 | 3.08 | 2.59 | zinc finger protein 761 | 388561 | NM_001008401 | Hs.433293 | ENSG00000160336 |
| ZNF785 | 2.85 | 2.5 | zinc finger protein 785 | 146540 | NM_152458 | Hs.513509 | ENSG00000197162 |
| ZNF793 | 3.98 | 3.26 | zinc finger protein 793 | 390927 | NM_001013659 | Hs.568010 | ENSG00000188227 |
| ZNF814 | 2.58 | 2.29 | zinc finger protein 814 | 730051 | NM_001144989 | Hs.634143 | ENSG00000204514 |
| ZNF818P | 3.18 | 2.52 | zinc finger protein 818, | 390963 | NM_001001675 | Hs.444446 | |
| | | | pseudogene | | | | |
| ZNF850 | 3.21 | 2.68 | zinc finger protein 850 | 342892 | NM_001193552 | Hs.406307 | ENSG00000267041 |
| ZNRF3-AS1 | 4.24 | 3.54 | ZNRF3 antisense RNA 1 | 100874 123 | NR_046851 | Hs.674708 | ENSG00000177993 |
| ZSCAN22 | 3.74 | 2.67 | zinc finger and SCAN | 342945 | NM_181846 | Hs.388162 | ENSG00000182318 |
| | | | domain containing 22 | | | | |
| ZYG11A | 4.09 | 3.51 | zyg-11 family member A, | 440590 | NM_001004339 | Hs.658458 | ENSG00000203995 |
| | | | cell cycle regulator | | | | |

TABLE 17A
Monocyte Subtype Genes.

| Fold- | | | | | | |
|---|---|---|---|---|---|---|
| change | | | | | | |
| mono1 vs | Unique | | Entrez | | | |
| mono2 | ID | Name | ID | Accession | UGCluster | Ensembl |
| −2.1276596 | NT5DC3 | 5′-nucleotidase domain | 51559 | NM_001031701 | Hs.48428 | ENSG00000111696 |
| | | containing 3 | | | | |
| 2.0400000 | PGLS | 6-phosphogluconolactonase | 25796 | NM_012088 | Hs.466165 | ENSG00000130313 |
| 2.0200000 | ABHD14A | abhydrolase domain | 25864 | NM_015407 | Hs.534400 | ENSG00000248487 |
| | | containing 14A | | | | |
| 2.0600000 | AAAS | achalasia, adrenocortical | 8086 | NM_001173466 | Hs.369144 | ENSG00000094914 |
| | | insufficiency, alacrimia | | | | |
| 1.7100000 | ACP2 | acid phosphatase 2, lysosomal | 53 | NM_001131064 | Hs.532492 | ENSG00000134575 |
| 1.5600000 | ACO2 | aconitase 2, mitochondrial | 50 | NM_001098 | Hs.643610 | ENSG00000100412 |
| 1.6400000 | APEH | acylaminoacyl-peptide | 327 | NM_001640 | Hs.517969 | ENSG00000164062 |
| | | hydrolase | | | | |
| −2.7027027 | ACBD7 | acyl-CoA binding domain | 414149 | NM_001039844 | Hs.644598 | ENSG00000176244 |
| | | containing 7 | | | | |
| −2.1276596 | ACADSB | acyl-CoA dehydrogenase, | 36 | NM_001609 | Hs.81934 | ENSG00000196177 |
| | | short/branched chain | | | | |
| 1.6300000 | AP1M1 | adaptor-related protein | 8907 | NM_001130524 | Hs.71040 | ENSG00000072958 |
| | | complex 1, mu 1 subunit | | | | |
| −2.3809524 | AP1S3 | adaptor-related protein | 130340 | NM_001039569 | Hs.632555 | ENSG00000152056 |
| | | complex 1, sigma 3 subunit | | | | |
| 1.4800000 | AP2B1 | adaptor-related protein | 163 | NM_001030006 | Hs.514819 | ENSG00000006125 |
| | | complex 2, beta 1 subunit | | | | |
| −1.7857143 | AP4S1 | adaptor-related protein | 11154 | NM_001128126 | Hs.293411 | ENSG00000100478 |
| | | complex 4, sigma 1 subunit | | | | |
| −1.7543860 | ADAT1 | adenosine deaminase, tRNA- | 23536 | NM_012091 | Hs.729312 | ENSG00000065457 |
| | | specific 1 | | | | |
| 1.5200000 | ADSL | adenylosuccinate lyase | 158 | NM_000026 | Hs.75527 | ENSG00000239900 |
| 1.5600000 | ARL2BP | ADP-ribosylation factor-like | 23568 | NM_012106 | Hs.632873 | ENSG00000102931 |
| | | 2 binding protein | | | | |
| −2.4390244 | ADRA1A | adrenoceptor alpha 1A | 148 | NM_000680 | Hs.709175 | ENSG00000120907 |
| −1.8181818 | AARS2 | alanyl-tRNA synthetase 2, | 57505 | NM_020745 | Hs.158381 | ENSG00000124608 |
| | | mitochondrial | | | | |
| 1.7400000 | ALDH2 | aldehyde dehydrogenase 2 | 217 | NM_000690 | Hs.604551 | ENSG00000111275 |
| | | family (mitochondrial) | | | | |
| −2.0408163 | ALDH6A1 | aldehyde dehydrogenase 6 | 4329 | NM_001278593 | Hs.293970 | ENSG00000119711 |
| | | family, member A1 | | | | |
| 1.5700000 | AKR1B1 | aldo-keto reductase family 1, | 231 | NM_001628 | Hs.521212 | ENSG00000085662 |
| | | member B1 (aldose | | | | |
| | | reductase) | | | | |
| 1.5800000 | ALKBH5 | AlkB family member 5, RNA | 54890 | NM_017758 | Hs.744130 | ENSG00000091542 |
| | | demethylase | | | | |
| −1.5384615 | ALPK1 | alpha-kinase 1 | 80216 | NM_001102406 | Hs.652825 | ENSG00000073331 |
| −2.0408163 | AASS | aminoadipate-semialdehyde | 10157 | NM_005763 | Hs.156738 | ENSG00000008311 |
| | | synthase | | | | |
| 2.0800000 | AGTRAP | angiotensin II receptor- | 57085 | NM_001040194 | Hs.464438 | ENSG00000177674 |
| | | associated protein | | | | |
| −1.8181818 | ASB11 | ankyrin repeat and SOCS box | 140456 | NM_001012428 | Hs.352183 | ENSG00000165192 |
| | | containing 11, E3 ubiquitin | | | | |
| | | protein ligase | | | | |
| −2.4390244 | ANKRD20A9P | ankyrin repeat domain 20 | 284232 | NR_027995 | Hs.679496 | |
| | | family, member A9, | | | | |
| | | pseudogene | | | | |
| −1.6949153 | ANKRD36B | ankyrin repeat domain 36B | 57730 | NM_020970 | Hs.532921 | ENSG00000196912 |
| 1.3900000 | ANXA11 | annexin A11 | 311 | NM_001157 | Hs.530291 | ENSG00000122359 |
| −1.6393443 | ANP32A-IT1 | ANP32A intronic transcript 1 | 80035 | NM_001040150 | Hs.662150 | |
| −1.3698630 | LAK1 | AP2 associated kinase 1 | 22848 | NM_014911 | Hs.468878 | ENSG00000115977 |
| −3.1250000 | APOBEC3B-AS1 | APOBEC3B antisense RNA | 100874530 | NR_104187 | Hs.626951 | ENSG00000249310 |
| | | 1 | | | | |
| 1.7200000 | APOA1BP | apolipoprotein A-I binding | 128240 | NM_144772 | Hs.528320 | ENSG00000163382 |
| | | protein | | | | |
| −3.8461538 | APOA2 | apolipoprotein A-II | 336 | NM_001643 | Hs.237658 | ENSG00000158874 |
| −1.8181818 | APOL1 | apolipoprotein L, 1 | 8542 | NM_001136540 | Hs.114309 | ENSG00000100342 |
| −2.4390244 | APOL4 | apolipoprotein L, 4 | 80832 | NM_030643 | Hs.11509 | ENSG00000100336 |
| −1.9607843 | ARGFX | arginine-fifty homeobox | 503582 | NM_001012659 | Hs.224976 | ENSG00000186103 |
| −2.8571429 | ARHGEF26-AS1 | ARHGEF26 antisense RNA 1 | 100507524 | NR_037901 | Hs.370221 | ENSG00000243069 |
| −1.8518519 | ARMC9 | armadillo repeat containing 9 | 80210 | NM_001271466 | Hs.471610 | ENSG00000135931 |
| 1.5400000 | ASNA1 | arsA arsenite transporter, | 439 | NM_004317 | Hs.465985 | ENSG00000198356 |
| | | ATP-binding, homolog 1 | | | | |
| | | (bacterial) | | | | |
| −5.2631579 | AS3MT | arsenite methyltransferase | 57412 | NM_020682 | Hs.720370 | ENSG00000214435 |
| −2.0833333 | AIPL1 | aryl hydrocarbon receptor | 23746 | NM_001033054 | Hs.279887 | ENSG00000129221 |
| | | interacting protein-like 1 | | | | |
| 1.5600000 | ASTE1 | asteroid homolog 1 | 28990 | NM_001288950 | Hs.100878 | ENSG00000034533 |
| | | (Drosophila) | | | | |
| −3.4482759 | ASTN2 | astrotactin 2 | 23245 | NM_001184734 | Hs.601562 | ENSG00000148219 |
| −1.4285714 | ATXN7 | ataxin 7 | 6314 | NM_000333 | Hs.476595 | ENSG00000163635 |
| 1.5400000 | ATP5B | ATP synthase, H+ | 506 | NM_001686 | Hs.406510 | ENSG00000110955 |
| | | transporting, mitochondrial | | | | |
| | | F1 complex, beta polypeptide | | | | |
| 2.5900000 | ATP5D | ATP synthase, H+ | 513 | NM_001001975 | Hs.418668 | ENSG00000099624 |
| | | transporting, mitochondrial | | | | |
| | | F1 complex, delta subunit | | | | |
| 2.5100000 | ATP5G2 | ATP synthase, H+ | 517 | NM_001002031 | Hs.524464 | ENSG00000135390 |
| | | transporting, mitochondrial | | | | |
| | | Fo complex, subunit C2 | | | | |
| | | (subunit 9) | | | | |
| −2.2222222 | ATAD3C | ATPase family, AAA domain | 219293 | NM_001039211 | Hs.724767 | ENSG00000215915 |
| | | containing 3C | | | | |
| −2.1739130 | ABCA9 | ATP-binding cassette, sub- | 10350 | NM_080283 | Hs.131686 | ENSG00000154258 |
| | | family A (ABC1), member 9 | | | | |
| −2.8571429 | ABCC9 | ATP-binding cassette, sub- | 10060 | NM_005691 | Hs.732701 | ENSG00000069431 |
| | | family C (CFTR/MRP), | | | | |
| | | member 9 | | | | |
| −1.7241379 | ABCF1 | ATP-binding cassette, sub- | 23 | NM_001025091 | Hs.655285 | ENSG00000204574 |
| | | family F (GCN20), member 1 | | | | |
| −2.5641026 | BBS5 | Bardet-Biedl syndrome 5 | 129880 | NM_152384 | Hs.233398 | ENSG00000163093 |
| −2.3255814 | BNIPL | BCL2/adenovirus E1B 19 kD | 149428 | NM_001159642 | Hs.591473 | ENSG00000163141 |
| | | interacting protein like | | | | |
| −2.0833333 | BCL2L2-PABPN1 | BCL2L2-PABPN1 | 100529063 | NM_001199864 | Hs.707712 | ENSG00000258643 |
| | | readthrough | | | | |
| −2.3809524 | BZRAP1 | benzodiazepine receptor | 9256 | NM_001261835 | Hs.112499 | ENSG00000005379 |
| | | (peripheral) associated | | | | |
| | | protein 1 | | | | |
| −2.2222222 | BHMT2 | betaine--homocysteine S- | 23743 | NM_001178005 | Hs.114172 | ENSG00000132840 |
| | | methyltransferase 2 | | | | |
| −1.7857143 | BVES | blood vessel epicardial | 11149 | NM_001199563 | Hs.221660 | ENSG00000112276 |
| | | substance | | | | |
| −2.0000000 | BMS1P4 | BMS1 pseudogene 4 | 729096 | NR_026592 | Hs.709171 | ENSG00000271816 |
| −2.0000000 | BMS1P5 | BMS1 pseudogene 5 | 399761 | NM_001040053 | Hs.571994 | ENSG00000204177 |
| −1.7857143 | BMS1P6 | BMS1 pseudogene 6 | 642826 | NR_024495 | Hs.463017 | ENSG00000251079 |
| 1.9300000 | BOLA3 | bolA family member 3 | 388962 | NM_001035505 | Hs.61472 | ENSG00000163170 |
| −2.1276596 | BMP7 | bone morphogenetic protein 7 | 655 | NM_001719 | Hs.473163 | ENSG00000101144 |
| 1.8200000 | BCKDHA | branched chain keto acid | 593 | NM_000709 | Hs.433307 | ENSG00000248098 |
| | | dehydrogenase E1, alpha | | | | |
| | | polypeptide | | | | |
| −2.3809524 | BRIP1 | BRCA1 interacting protein C- | 83990 | NM_032043 | Hs.128903 | ENSG00000136492 |
| | | terminal helicase 1 | | | | |
| −2.6315789 | BREA2 | breast cancer estrogen- | 286076 | NM_001024610 | Hs.178095 | ENSG00000181097 |
| | | induced apoptosis 2 | | | | |
| −1.4492754 | BAZ2A | bromodomain adjacent to | 11176 | NM_001300905 | Hs.314263 | ENSG00000076108 |
| | | zinc finger domain, 2A | | | | |
| −1.7543860 | BTN2A1 | butyrophilin, subfamily 2, | 11120 | NM_001197233 | Hs.159028 | ENSG00000112763 |
| | | member A1 | | | | |
| 2.5600000 | C10orf32-ASMT | C10orf32-ASMT readthrough | 100528007 | NR_037644 | Hs.720370 | |
| | | (NMD candidate) | | | | |
| −1.7241379 | CIRL-AS1 | CIRL antisense RNA 1 | 283314 | NR_026947 | Hs.744212 | ENSG00000205885 |
| −2.0408163 | CDH23 | cadherin-related 23 | 64072 | NM_001171930 | Hs.656032 | ENSG00000107736 |
| −2.1739130 | LOC729603 | calcineurin-like EF-hand | 729603 | NR_003288 | Hs.674810 | ENSG00000213073 |
| | | protein 1 pseudogene | | | | |
| −1.8867925 | CAMK1D | calcium/calmodulin- | 57118 | NM_020397 | Hs.600547 | ENSG00000183049 |
| | | dependent protein kinase ID | | | | |
| 1.3600000 | CAD | carbamoyl-phosphate | 790 | NM_004341 | Hs.377010 | ENSG00000084774 |
| | | synthetase 2, aspartate | | | | |
| | | transcarbamylase, and | | | | |
| | | dihydroorotase | | | | |
| −2.1739130 | CHST6 | carbohydrate (N- | 4166 | NM_021615 | Hs.655622 | ENSG00000183196 |
| | | acetylglucosamine 6-O) | | | | |
| | | sulfotransferase 6 | | | | |
| −1.8867925 | CA5B | carbonic anhydrase VB, | 11238 | NM_00722 | Hs.653287 | ENSG00000169239 |
| | | mitochondrial | | | | |
| 1.3200000 | CRTAP | cartilage associated protein | 10491 | NM_006371 | Hs.517888 | ENSG00000170275 |
| 1.6700000 | CASD1 | CAS1 domain containing 1 | 64921 | NM_022900 | Hs.260041 | ENSG00000127995 |
| 1.3600000 | CSNK2A1 | casein kinase 2, alpha 1 | 1457 | NM_001895 | Hs.644056 | ENSG00000101266 |
| | | polypeptide | | | | |
| −1.5873016 | CFLAR | CASP8 and FADD-like | 8837 | NM_001127183 | Hs.390736 | ENSG00000003402 |
| | | apoptosis regulator | | | | |
| 1.8600000 | CAT | catalase | 847 | NM_001752 | Hs.502302 | ENSG00000121691 |
| 1.5900000 | COMT | catechol-O-methyltransferase | 1312 | NM_000754 | Hs.370408 | ENSG00000093010 |
| −1.5873016 | CTNNBL1 | catenin, beta like 1 | 56259 | NM_001281495 | Hs.472667 | ENSG00000132792 |
| 1.3200000 | CD164 | CD164 molecule, sialomucin | 8763 | NM_001142401 | Hs.520313 | ENSG00000135535 |
| −2.3809524 | CD24 | CD24 molecule | 100133941 | NM_001291737 | Hs.644105 | ENSG00000272398 |
| −2.5641026 | CDKN2B-AS1 | CDKN2B antisense RNA 1 | 100048912 | NR_003529 | Hs.493614 | ENSG00000240498 |
| 2.2400000 | CDIPT | CDP-diacylglycerol--inositol | 10423 | NM_001286585 | Hs.121549 | ENSG00000103502 |
| | | 3-phosphatidyltransferase | | | | |
| −2.5641026 | LOC101409256 | cell division cycle 42 | 101409256 | NR_102424 | | |
| | | pseudogene | | | | |
| −1.4492754 | CCAR1 | cell division cycle and | 55749 | NM_001282959 | Hs.49853 | ENSG00000060339 |
| | | apoptosis regulator 1 | | | | |
| 1.8200000 | CREG1 | cellular repressor of E1A- | 8804 | NM_003851 | Hs.5710 | ENSG00000143162 |
| | | stimulated genes 1 | | | | |
| −1.5625000 | CENPC | centromere protein C | 1060 | NM_001812 | Hs.479867 | ENSG00000145241 |
| −2.0408163 | CEP41 | centrosomal protein 41 kDa | 95681 | NM_001257158 | Hs.368315 | ENSG00000106477 |
| −1.2820513 | CERS5 | ceramide synthase 5 | 91012 | NM_001281731 | Hs.270525 | ENSG00000139624 |
| 1.5700000 | CCM2 | cerebral cavernous | 83605 | NM_001029835 | Hs.148272 | ENSG00000136280 |
| | | malformation 2 | | | | |
| 1.8600000 | CLN6 | ceroid-lipofuscinosis, | 54982 | NM_017882 | Hs.584921 | ENSG00000128973 |
| | | neuronal 6, late infantile, | | | | |
| | | variant | | | | |
| −1.3888889 | CHMP1B | charged multivesicular body | 57132 | NM_020412 | Hs.656244 | ENSG00000255112 |
| | | protein 1B | | | | |
| 1.2600000 | CHMP3 | charged multivesicular body | 51652 | NM_001005753 | Hs.591582 | ENSG00000115561 |
| | | protein 3 | | | | |
| −2.3255814 | CCR6 | chemokine (C-C motif) | 1235 | NM_004367 | Hs.46468 | ENSG00000112486 |
| | | receptor 6 | | | | |
| −2.5641026 | CHRM3 | cholinergic receptor, | 1131 | NM_000740 | Hs.7138 | ENSG00000133019 |
| | | muscarinic 3 | | | | |
| −1.8181818 | CHRNB1 | cholinergic receptor, | 1140 | NM_000747 | Hs.330386 | ENSG00000170175 |
| | | nicotinic, beta 1 (muscle) | | | | |
| −2.2727273 | LOC440300 | chondroitin sulfate | 440300 | NR_033738 | Hs.546565 | ENSG00000259295 |
| | | proteoglycan 4 pseudogene | | | | |
| −1.4705882 | CBX5 | chromobox homolog 5 | 23468 | NM_001127321 | Hs.349283 | ENSG00000094916 |
| 2.6700000 | C1orf122 | chromosome 1 open reading | 127687 | NM_001142726 | Hs.532749 | ENSG00000197982 |
| | | frame 122 | | | | |
| −1.8867925 | C1orf174 | chromosome 1 open reading | 339448 | NM_207356 | Hs.103939 | ENSG00000198912 |
| | | frame 174 | | | | |
| −2.9411765 | C1orf229 | chromosome 1 open reading | 388759 | NM_207401 | Hs.456511 | ENSG00000221953 |
| | | frame 229 | | | | |
| −1.4492754 | C1orf27 | chromosome 1 open reading | 54953 | NM_001164245 | Hs.371210 | ENSG00000157181 |
| | | frame 27 | | | | |
| −1.6129032 | C10orf32 | chromosome 10 open reading | 119032 | NM_001136200 | Hs.34492 | ENSG00000166275 |
| | | frame 32 | | | | |
| 1.5400000 | C12orf49 | chromosome 12 open reading | 79794 | NM_024738 | Hs.592011 | ENSG00000111412 |
| | | frame 49 | | | | |
| −1.4492754 | C12orf5 | chromosome 12 open reading | 57103 | NM_020375 | Hs.504545 | ENSG00000078237 |
| | | frame 5 | | | | |
| 1.6400000 | C14orf142 | chromosome 14 open reading | 84520 | NM_032490 | Hs.20142 | ENSG00000170270 |
| | | frame 142 | | | | |
| 2.3400000 | C16orf54 | chromosome 16 open reading | 283897 | NM_175900 | Hs.331095 | ENSG00000185905 |
| | | frame 54 | | | | |
| 1.5100000 | C16orf62 | chromosome 16 open reading | 57020 | NM_001300743 | Hs.654964 | ENSG00000103544 |
| | | frame 62 | | | | |
| −2.0000000 | C17orf75 | chromosome 17 open reading | 64149 | NM_022344 | Hs.655257 | ENSG00000108666 |
| | | frame 75 | | | | |
| −2.0000000 | C17orf77 | chromosome 17 open reading | 146723 | NM_001302809 | Hs.350775 | ENSG00000182352 |
| | | frame 77 | | | | |
| 2.5600000 | C19orf24 | chromosome 19 open reading | 55009 | NM_017914 | Hs.591383 | ENSG00000228300 |
| | | frame 24 | | | | |
| −2.2727273 | C19orf40 | chromosome 19 open reading | 91442 | NM_001300978 | Hs.579899 | ENSG00000131944 |
| | | frame 40 | | | | |
| 2.2300000 | C19orf70 | chromosome 19 open reading | 125988 | NM_205767 | Hs.356626 | ENSG00000174917 |
| | | frame 70 | | | | |
| −2.0000000 | C2orf83 | chromosome 2 open reading | 56918 | NM_001162483 | Hs.283092 | ENSG00000042304 |
| | | frame 83 | | | | |
| −3.1250000 | C2orf91 | chromosome 2 open reading | 400950 | NM_001242815 | Hs.738713 | ENSG00000205086 |
| | | frame 91 | | | | |
| −2.7777778 | C20orf203 | chromosome 20 open reading | 284805 | NM_182584 | Hs.353262 | |
| | | frame 203 | | | | |
| 3.1100000 | C20orf27 | chromosome 20 open reading | 54976 | NM_001039140 | Hs.274422 | ENSG00000101220 |
| | | frame 27 | | | | |
| −2.3809524 | C21orf62 | chromosome 21 open reading | 56245 | NM_001162495 | Hs.517235 | ENSG00000262938 |
| | | frame 62 | | | | |
| −2.0000000 | C3orf33 | chromosome 3 open reading | 285315 | NM_173657 | Hs.350846 | ENSG00000174928 |
| | | frame 33 | | | | |
| −1.8518519 | C4orf19 | chromosome 4 open reading | 55286 | NM_001104629 | Hs.107527 | ENSG00000154274 |
| | | frame 19 | | | | |
| −2.2727273 | C4orf26 | chromosome 4 open reading | 152816 | NM_001206981 | Hs.24510 | ENSG00000174792 |
| | | frame 26 | | | | |
| −1.9607843 | C5orf28 | chromosome 5 open reading | 64417 | NM_022483 | Hs.732093 | ENSG00000151881 |
| | | frame 28 | | | | |
| −1.9230769 | C5orf66 | chromosome 5 open reading | 100996485 | NM_001277348 | | ENSG00000224186 |
| | | frame 66 | | | | |
| 1.9400000 | C6orf1 | chromosome 6 open reading | 221491 | NM_001008703 | Hs.381300 | ENSG00000186577 |
| | | frame 1 | | | | |
| 1.4400000 | C6orf120 | chromosome 6 open reading | 387263 | NM_001029863 | Hs.591375 | ENSG00000185127 |
| | | frame 120 | | | | |
| 1.9100000 | C7orf26 | chromosome 7 open reading | 79034 | NM_001303039 | Hs.487511 | ENSG00000146576 |
| | | frame 26 | | | | |
| 2.9900000 | C9orf16 | chromosome 9 open reading | 79095 | NM_024112 | Hs.522412 | ENSG00000171159 |
| | | frame 16 | | | | |
| −1.6129032 | C9orf3 | chromosome 9 open reading | 84909 | NM_001193329 | Hs.434253 | ENSG00000148120 |
| | | frame 3 | | | | |
| 2.2200000 | C9orf69 | chromosome 9 open reading | 90120 | NM_001256526 | Hs.287411 | ENSG00000238227 |
| | | frame 69 | | | | |
| −2.2222222 | CXorf36 | chromosome X open reading | 79742 | NM_024689 | Hs.98321 | ENSG00000147113 |
| | | frame 36 | | | | |
| −2.1276596 | CXorf56 | chromosome X open reading | 63932 | NM_001170569 | Hs.248572 | ENSG00000018610 |
| | | frame 56 | | | | |
| −1.7543860 | CROCCP3 | ciliary rootlet coiled-coil, | 114819 | NR_023386 | Hs.597881 | ENSG00000080947 |
| | | rootletin pseudogene 3 | | | | |
| 1.6000000 | CMTM3 | CKLF-like MARVEL | 123920 | NM_001048251 | Hs.298198 | ENSG00000140931 |
| | | transmembrane domain | | | | |
| | | containing 3 | | | | |
| −2.0000000 | CKMT2-AS1 | CKMT2 antisense RNA 1 | 100131067 | NR_034121 | Hs.655855 | ENSG00000247572 |
| −1.8181818 | CLSPN | claspin | 63967 | NM_001190481 | Hs.175613 | ENSG00000092853 |
| −1.4705882 | CLINT1 | clathrin interactor 1 | 9685 | NM_001195555 | Hs.644000 | ENSG00000113282 |
| 1.7300000 | CLDN15 | claudin 15 | 24146 | NM_001185080 | Hs.38738 | ENSG00000106404 |
| −1.7543860 | CLDN19 | claudin 19 | 149461 | NM_001123395 | Hs.496270 | ENSG00000164007 |
| 1.5300000 | COPZ1 | coatomer protein complex, | 22818 | NM_001271734 | Hs.505652 | ENSG00000111481 |
| | | subunit zeta 1 | | | | |
| 1.4800000 | CCDC115 | coiled-coil domain containing | 84317 | NM_032357 | Hs.104203 | ENSG00000136710 |
| | | 115 | | | | |
| −1.9230769 | CCDC12 | coiled-coil domain containing | 151903 | NM_001277074 | Hs.631918 | ENSG00000160799 |
| | | 12 | | | | |
| −2.0408163 | CCDC144B | coiled-coil domain containing | 284047 | NR_036647 | Hs.448012 | ENSG00000154874 |
| | | 144B (pseudogene) | | | | |
| −2.0833333 | CCDC148 | coiled-coil domain containing | 130940 | NM_001171637 | Hs.668597 | ENSG00000153237 |
| | | 148 | | | | |
| −2.0000000 | CCBE1 | collagen and calcium binding | 147372 | NM_133459 | Hs.34333 | ENSG00000183287 |
| | | EGF domains 1 | | | | |
| −1.7543860 | COMMD2 | COMM domain containing 2 | 51122 | NM_016094 | Hs.432729 | ENSG00000114744 |
| 1.7000000 | COMMD5 | COMM domain containing 5 | 28991 | NM_001081003 | Hs.631856 | ENSG00000170619 |
| 1.6700000 | C1QBP | complement component 1, q | 708 | NM_001212 | Hs.555866 | ENSG00000108561 |
| | | subcomponent binding | | | | |
| | | protein | | | | |
| −2.7027027 | CRX | cone-rod homeobox | 1406 | NM_000554 | Hs.617342 | ENSG00000105392 |
| 1.7300000 | CPNE1 | copine I | 8904 | NM_001198863 | Hs.246413 | ENSG00000214078 |
| 2.1400000 | CORO1A | coronin, actin binding | 11151 | NM_001193333 | Hs.415067 | ENSG00000102879 |
| | | protein, 1A | | | | |
| −2.0000000 | CXADR | coxsackie virus and | 1525 | NM_001207063 | Hs.627078 | ENSG00000154639 |
| | | adenovirus receptor | | | | |
| −2.1739130 | CRYM-AS1 | CRYM antisense RNA 1 | 400508 | NM_001101368 | Hs.578949 | |
| −2.4390244 | CRYBB2P1 | crystallin, beta B2 | 1416 | NR_033733 | Hs.571835 | ENSG00000100058 |
| | | pseudogene 1 | | | | |
| 1.6400000 | CHTF8 | CTF8, chromosome | 54921 | NM_001002847 | Hs.85962 | ENSG00000168802 |
| | | transmission fidelity factor 8 | | | | |
| | | homolog (S. cerevisiae) | | | | |
| −1.7857143 | CLEC2D | C-type lectin domain family | 29121 | NM_001004419 | Hs.268326 | ENSG00000069493 |
| | | 2, member D | | | | |
| 1.7000000 | CLEC4A | C-type lectin domain family | 50856 | NM_016184 | Hs.504657 | ENSG00000111729 |
| | | 4, member A | | | | |
| −1.8518519 | CWC25 | CWC25 spliceosome- | 54883 | NM_017748 | Hs.406223 | ENSG00000273559 |
| | | associated protein homolog | | | | |
| | | (S. cerevisiae) | | | | |
| −1.3698630 | DMTF1 | cyclin D binding myb-like | 9988 | NM_001142326 | Hs.196129 | ENSG00000135164 |
| | | transcription factor 1 | | | | |
| −1.2987013 | CCNT2 | cyclin T2 | 905 | NM_001241 | Hs.744115 | ENSG00000082258 |
| −2.2727273 | LOC727896 | cysteine and histidine-rich | 727896 | NR_026659 | Hs.673126 | |
| | | domain (CHORD) containing | | | | |
| | | 1 pseudogene | | | | |
| −1.9607843 | CSAD | cysteine sulfinic acid | 51380 | NM_001244705 | Hs.279815 | ENSG00000139631 |
| | | decarboxylase | | | | |
| 2.5200000 | CDA | cytidine deaminase | 978 | NM_001785 | Hs.466910 | ENSG00000158825 |
| 1.8900000 | CYB561D2 | cytochrome b561 family, | 11068 | NM_001291284 | Hs.149443 | ENSG00000114395 |
| | | member D2 | | | | |
| −2.0000000 | COA7 | cytochrome c oxidase | 65260 | NM_023077 | Hs.349905 | ENSG00000162377 |
| | | assembly factor 7 (putative) | | | | |
| −2.9411765 | COX6B2 | cytochrome c oxidase subunit | 125965 | NM_144613 | Hs.550544 | ENSG00000160471 |
| | | VIb polypeptide 2 (testis) | | | | |
| 2.3100000 | COX8A | cytochrome c oxidase subunit | 1351 | NM_004074 | Hs.743989 | ENSG00000176340 |
| | | VIIIA (ubiquitous) | | | | |
| −1.4925373 | CYCS | cytochrome c, somatic | 54205 | NM_018947 | Hs.437060 | ENSG00000172115 |
| 1.6800000 | CYC1 | cytochrome c-1 | 1537 | NM_001916 | Hs.289271 | ENSG00000179091 |
| −1.6393443 | CYP20A1 | cytochrome P450, family 20, | 57404 | NM_020674 | Hs.446065 | ENSG00000119004 |
| | | subfamily A, polypeptide 1 | | | | |
| −1.8867925 | CYP4V2 | cytochrome P450, family 4, | 285440 | NM_207352 | Hs.587231 | ENSG00000145476 |
| | | subfamily V, polypeptide 2 | | | | |
| −1.8181818 | CYP51A1 | cytochrome P450, family 51, | 1595 | NM_000786 | Hs.417077 | ENSG00000001630 |
| | | subfamily A, polypeptide 1 | | | | |
| −1.6129032 | CYTIP | cytohesin 1 interacting | 9595 | NM_004288 | Hs.270 | ENSG00000115165 |
| | | protein | | | | |
| −2.5000000 | CRLF2 | cytokine receptor-like factor | 64109 | NM_001012288 | Hs.287729 | ENSG00000205755 |
| | | 2 | | | | |
| −1.7857143 | CRTAM | cytotoxic and regulatory T | 56253 | NM_001304782 | Hs.159523 | ENSG00000109943 |
| | | cell molecule | | | | |
| 2.1800000 | DBP | D site of albumin promoter | 1628 | NM_001352 | Hs.414480 | ENSG00000105516 |
| | | (albumin D-box) binding | | | | |
| | | protein | | | | |
| 1.5100000 | DDB1 | damage-specific DNA | 1642 | NM_001923 | Hs.290758 | ENSG00000167986 |
| | | binding protein 1, 127 kDa | | | | |
| −1.6393443 | DZIP3 | DAZ interacting zinc finger | 9666 | NM_014648 | Hs.409210 | ENSG00000198919 |
| | | protein 3 | | | | |
| −2.3255814 | DCUN1D2 | DCN1, defective in cullin | 55208 | NM_001014283 | Hs.682987 | ENSG00000150401 |
| | | neddylation 1, domain | | | | |
| | | containing 2 | | | | |
| −1.4084507 | DCAF16 | DDB1 and CUL4 associated | 54876 | NM_017741 | Hs.614787 | ENSG00000163257 |
| | | factor 16 | | | | |
| 1.9500000 | DDT | D-dopachrome tautomerase | 1652 | NM_001084392 | Hs.656723 | ENSG00000099977 |
| −1.8518519 | DDX51 | DEAD (Asp-Glu-Ala-Asp) | 317781 | NM_175066 | Hs.445168 | ENSG00000185163 |
| | | (SEQ ID NO: 801) box | | | | |
| | | polypeptide 51 | | | | |
| 1.5500000 | DEDD | death effector domain | 9191 | NM_001039711 | Hs.744092 | ENSG00000158796 |
| | | containing | | | | |
| −1.6666667 | DLEU1 | deleted in lymphocytic | 10301 | NM_005887 | Hs.591229 | ENSG00000176124 |
| | | leukemia 1 (non-protein | | | | |
| | | coding) | | | | |
| −2.2222222 | DLK2 | delta-like 2 homolog | 65989 | NM_001286655 | Hs.337251 | ENSG00000171462 |
| | | (Drosophila) | | | | |
| −1.4492754 | DENND6A | DENN/MADD domain | 201627 | NM_152678 | Hs.91085 | ENSG00000174839 |
| | | containing 6A | | | | |
| −2.2222222 | DSG2 | desmoglein 2 | 1829 | NM_001943 | Hs.412597 | ENSG00000046604 |
| −2.3255814 | DSG3 | desmoglein 3 | 1830 | NM_001944 | Hs.1925 | ENSG00000134757 |
| −1.2987013 | DIABLO | diablo, IAP-binding | 56616 | NM_001278302 | Hs.169611 | ENSG00000184047 |
| | | mitochondrial protein | | | | |
| 1.6600000 | DBI | diazepam binding inhibitor | 1622 | NM_001079862 | Hs.78888 | ENSG00000155368 |
| | | (GABA receptor modulator, | | | | |
| | | acyl-CoA binding protein) | | | | |
| −2.0000000 | DPH3P1 | diphthamide biosynthesis 3 | 100132911 | NM_080750 | | |
| | | pseudogene 1 | | | | |
| 1.6700000 | DPH7 | diphthamide biosynthesis 7 | 92715 | NM_138778 | Hs.292570 | ENSG00000148399 |
| −1.4492754 | DIS3 | DIS3 exosome | 22894 | NM_001128226 | Hs.744104 | ENSG00000083520 |
| | | endoribonuclease and 3′-5′ | | | | |
| | | exoribonuclease | | | | |
| −3.3333333 | DLGAP1-AS2 | DLGAP1 antisense RNA 2 | 84777 | NM_032691 | Hs.659053 | |
| −1.6666667 | DFFA | DNA fragmentation factor, | 1676 | NM_004401 | Hs.484782 | ENSG00000160049 |
| | | 45 kDa, alpha polypeptide | | | | |
| −2.2727273 | DMC1 | DNA meiotic recombinase 1 | 11144 | NM_001278208 | Hs.339396 | ENSG00000100206 |
| −1.8518519 | DNAH17-AS1 | DNAH17 antisense RNA 1 | 100996295 | NR_102401 | Hs.615304 | ENSG00000267432 |
| −1.4285714 | DNAJC21 | DnaJ (Hsp40) homolog, | 134218 | NM_001012339 | Hs.131887 | ENSG00000168724 |
| | | subfamily C, member 21 | | | | |
| −2.5000000 | DNAJC22 | DnaJ (Hsp40) homolog, | 79962 | NM_001304944 | Hs.659300 | ENSG00000178401 |
| | | subfamily C, member 22 | | | | |
| 1.9400000 | DNAJC4 | DnaJ (Hsp40) homolog, | 3338 | NM_005528 | Hs.172847 | ENSG00000110011 |
| | | subfamily C, member 4 | | | | |
| −2.0408163 | DNAJC9-AS1 | DNAJC9 antisense RNA 1 | 414245 | NR_038373 | Hs.661857 | ENSG00000236756 |
| 2.3400000 | DNLZ | DNL-type zinc finger | 728489 | NM_001080849 | Hs.528581 | ENSG00000213221 |
| −1.8518519 | DNM1P46 | DNM1 pseudogene 46 | 196968 | NM_194295 | Hs.567763 | ENSG00000182397 |
| −2.2222222 | DUXA | double homeobox A | 503835 | NM_001012729 | Hs.585857 | ENSG00000258873 |
| −2.5641026 | DYDC1 | DPY30 domain containing 1 | 143241 | NM_001269053 | Hs.407751 | ENSG00000170788 |
| −1.6129032 | DSTYK | dual serine/threonine and | 25778 | NM_015375 | Hs.6874 | ENSG00000133059 |
| | | tyrosine protein kinase | | | | |
| 2.1000000 | DUSP23 | dual specificity phosphatase | 54935 | NM_017823 | Hs.425801 | ENSG00000158716 |
| | | 23 | | | | |
| 1.7200000 | DUSP7 | dual specificity phosphatase 7 | 1849 | NM_001947 | Hs.591664 | ENSG00000164086 |
| 2.2900000 | DYRK1B | dual-specificity tyrosine-(Y)- | 9149 | NM_004714 | Hs.130988 | ENSG00000105204 |
| | | phosphorylation regulated | | | | |
| | | kinase 1B | | | | |
| 1.6400000 | DCTN2 | dynactin 2 (p50) | 10540 | NM_001261412 | Hs.289123 | ENSG00000175203 |
| −2.5641026 | DYNAP | dynactin associated protein | 284254 | NM_173629 | Hs.376146 | ENSG00000178690 |
| 1.5100000 | DNAAF2 | dynein, axonemal, assembly | 55172 | NM_001083908 | Hs.231761 | ENSG00000165506 |
| | | factor 2 | | | | |
| −1.9230769 | DNAL1 | dynein, axonemal, light chain 1 | 83544 | NM_001201366 | Hs.271270 | ENSG00000119661 |
| −1.9607843 | DYX1C1 | dyslexia susceptibility 1 | 161582 | NM_001033559 | Hs.126403 | ENSG00000256061 |
| | | candidate 1 | | | | |
| −1.6393443 | ENTPD4 | ectonucleoside triphosphate | 9583 | NM_001128930 | Hs.444389 | ENSG00000197217 |
| | | diphosphohydrolase 4 | | | | |
| −1.9607843 | EFCAB11 | EF-hand calcium binding | 90141 | NM_001284266 | Hs.123232 | ENSG00000140025 |
| | | domain 11 | | | | |
| −2.0833333 | EGFEM1P | EGF-like and EMI domain | 93556 | NR_021485 | Hs.478158 | ENSG00000206120 |
| | | containing 1, pseudogene | | | | |
| −1.6666667 | ERC1 | ELKS/RAB6- | 23085 | NM_001301248 | Hs.601216 | ENSG00000082805 |
| | | interacting/CAST family | | | | |
| | | member 1 | | | | |
| −2.1276596 | ELMOD1 | ELMO/CED-12 domain | 55531 | NM_001130037 | Hs.495779 | ENSG00000110675 |
| | | containing 1 | | | | |
| −3.2258065 | ESRG | embryonic stem cell related | 790952 | NR_027122 | Hs.720658 | ENSG00000265992 |
| | | (non-protein coding) | | | | |
| −1.7543860 | EBP | emopamil binding protein | 10682 | NM_006579 | Hs.30619 | ENSG00000147155 |
| | | (sterol isomerase) | | | | |
| −2.2222222 | EMX2OS | EMX2 opposite | 196047 | NR_002791 | Hs.312592 | ENSG00000229847 |
| | | strand/antisense RNA | | | | |
| −1.7543860 | ERVK13-1 | endogenous retrovirus group | 100507321 | NM_001012731 | Hs.406976 | ENSG00000260565 |
| | | K13, member 1 | | | | |
| −2.3809524 | ERVV-1 | endogenous retrovirus group | 147664 | NM_152473 | Hs.44329 | ENSG00000269526 |
| | | V, member 1 | | | | |
| 1.5000000 | ERH | enhancer of rudimentary | 2079 | NM_004450 | Hs.509791 | ENSG00000100632 |
| | | homolog (Drosophila) | | | | |
| 1.5800000 | ECH1 | enoyl CoA hydratase 1, | 1891 | NM_001398 | Hs.196176 | ENSG00000104823 |
| | | peroxisomal | | | | |
| −2.1276596 | ENTPD1-AS1 | ENTPD1 antisense RNA 1 | 728558 | NR_038444 | Hs.538374 | ENSG00000226688 |
| −2.5641026 | EPHA10 | EPH receptor A10 | 284656 | NM_001004338 | Hs.129435 | ENSG00000183317 |
| −2.6315789 | EPPIN | epididymal peptidase | 57119 | NM_001302861 | Hs.121084 | ENSG00000101448 |
| | | inhibitor | | | | |
| −1.8867925 | ECT2 | epithelial cell transforming 2 | 1894 | NM_001258315 | Hs.518299 | ENSG00000114346 |
| −2.0000000 | EMP2 | epithelial membrane protein 2 | 2013 | NM_001424 | Hs.531561 | ENSG00000213853 |
| 1.3500000 | EMC10 | ER membrane protein | 284361 | NM_175063 | Hs.448941 | ENSG00000161671 |
| | | complex subunit 10 | | | | |
| 4.5600000 | EMC6 | ER membrane protein | 83460 | NM_001014764 | Hs.30011 | ENSG00000127774 |
| | | complex subunit 6 | | | | |
| 1.6200000 | EMC8 | ER membrane protein | 10328 | NM_001142288 | Hs.173162 | ENSG00000131148 |
| | | complex subunit 8 | | | | |
| 1.9700000 | ERI3 | ERI1 exoribonuclease family | 79033 | NM_001301698 | Hs.731413 | ENSG00000117419 |
| | | member 3 | | | | |
| 2.3100000 | ETHE1 | ethylmalonic encephalopathy 1 | 23474 | NM_014297 | Hs.7486 | ENSG00000105755 |
| −1.3698630 | EEF1D | eukaryotic translation | 1936 | NM_001130053 | Hs.333388 | ENSG00000104529 |
| | | elongation factor 1 delta | | | | |
| | | (guanine nucleotide exchange | | | | |
| | | protein) | | | | |
| 2.2300000 | EIF4EBP3 | eukaryotic translation | 8637 | NM_003732 | Hs.594084 | ENSG00000243056 |
| | | initiation factor 4E binding | | | | |
| | | protein 3 | | | | |
| −1.5873016 | EIF5 | eukaryotic translation | 1983 | NM_001969 | Hs.433702 | ENSG00000100664 |
| | | initiation factor 5 | | | | |
| −1.3888889 | EWSR1 | EWS RNA-binding protein 1 | 2130 | NM_001163285 | Hs.374477 | ENSG00000182944 |
| −2.0408163 | EXPH5 | exophilin 5 | 23086 | NM_001144763 | Hs.28540 | ENSG00000110723 |
| 1.3800000 | ESYT1 | extended synaptotagmin-like | 23344 | NM_001184796 | Hs.632729 | ENSG00000139641 |
| | | protein 1 | | | | |
| −1.9230769 | FAM83H-AS1 | FAM83H antisense RNA 1 | 100128338 | NR_033849 | Hs.493171 | ENSG00000203499 |
| | | (head to head) | | | | |
| −1.8518519 | FAM114A1 | family with sequence | 92689 | NM_138389 | Hs.476517 | ENSG00000197712 |
| | | similarity 114, member A1 | | | | |
| −2.1739130 | FAM122C | family with sequence | 159091 | NM_001170779 | Hs.269127 | ENSG00000156500 |
| | | similarity 122C | | | | |
| −2.8571429 | FAM133DP | family with sequence | 728066 | NR_034169 | Hs.470311 | |
| | | similarity 133, member A | | | | |
| | | pseudogene | | | | |
| −1.4084507 | FAM177A1 | family with sequence | 283635 | NM_001079519 | Hs.446357 | ENSG00000151327 |
| | | similarity 177, member A1 | | | | |
| −1.9230769 | FAM71F2 | family with sequence | 346653 | NM_001012454 | Hs.445236 | ENSG00000205085 |
| | | similarity 71, member F2 | | | | |
| 1.7700000 | FAM89B | family with sequence | 23625 | NM_001098784 | Hs.731854 | ENSG00000176973 |
| | | similarity 89, member B | | | | |
| 1.6400000 | FAM96A | family with sequence | 84191 | NM_001014812 | Hs.439548 | ENSG00000166797 |
| | | similarity 96, member A | | | | |
| −2.2727273 | FDPSP2 | farnesyl diphosphate synthase | 619190 | NR_003262 | Hs.609978 | ENSG00000233980 |
| | | pseudogene 2 | | | | |
| 1.5000000 | FNTA | farnesyltransferase, CAAX | 2339 | NM_001018676 | Hs.370312 | ENSG00000168522 |
| | | box, alpha | | | | |
| −2.3809524 | FAIM | Fas apoptotic inhibitory | 55179 | NM_001033030 | Hs.173438 | ENSG00000158234 |
| | | molecule | | | | |
| −1.8867925 | FEZ1 | fasciculation and elongation | 9638 | NM_005103 | Hs.224008 | ENSG00000149557 |
| | | protein zeta 1 (zygin I) | | | | |
| 1.9500000 | FBXL14 | F-box and leucine-rich repeat | 144699 | NM_152441 | Hs.367956 | ENSG00000171823 |
| | | protein 14 | | | | |
| −1.8181818 | FBXL18 | F-box and leucine-rich repeat | 80028 | NM_024963 | Hs.623974 | ENSG00000155034 |
| | | protein 18 | | | | |
| −1.4492754 | FBXL20 | F-box and leucine-rich repeat | 84961 | NM_001184906 | Hs.462946 | ENSG00000108306 |
| | | protein 20 | | | | |
| 1.7200000 | FBXW5 | F-box and WD repeat domain | 54461 | NM_018998 | Hs.522507 | ENSG00000159069 |
| | | containing 5 | | | | |
| −2.0408163 | FBXO17 | F-box protein 17 | 115290 | NM_024907 | Hs.531770 | ENSG00000269190 |
| −2.2222222 | FBXO27 | F-box protein 27 | 126433 | NM_178820 | Hs.187461 | ENSG00000161243 |
| −1.6129032 | FBXO44 | F-box protein 44 | 93611 | NM_001014765 | Hs.556006 | ENSG00000132879 |
| 1.3800000 | FEM1A | fem-1 homolog a (C. elegans) | 55527 | NM_018708 | Hs.515082 | ENSG00000141965 |
| 1.6800000 | FDX1L | ferredoxin 1-like | 112812 | NM_001031734 | Hs.654865 | ENSG00000267673 |
| −2.2727273 | FRRS1 | ferric-chelate reductase 1 | 391059 | NM_001013660 | Hs.454779 | ENSG00000156869 |
| −1.8181818 | FGFR1OP | FGFR1 oncogene partner | 11116 | NM_001278690 | Hs.487175 | ENSG00000213066 |
| −2.5641026 | LOC100335030 | FGFR1 oncogene partner 2 | 100335030 | NR_033267 | Hs.687044 | ENSG00000257954 |
| | | pseudogene | | | | |
| −2.5000000 | FGF5 | fibroblast growth factor 5 | 2250 | NM_001291812 | Hs.37055 | ENSG00000138675 |
| −1.9607843 | FGFR2 | fibroblast growth factor | 2263 | NM_00014 | Hs.533683 | ENSG00000066468 |
| | | receptor 2 | | | | |
| −2.1739130 | FBLN1 | fibulin 1 | 2192 | NM_001996 | Hs.24601 | ENSG00000077942 |
| −3.4482759 | FILIP1 | filamin A interacting protein 1 | 27145 | NM_001289987 | Hs.696158 | ENSG00000118407 |
| −2.5641026 | FBLIM1 | filamin binding LIM protein 1 | 54751 | NM_001024215 | Hs.530101 | ENSG00000162458 |
| 1.8300000 | FIS1 | fission 1 (mitochondrial outer | 51024 | NM_016068 | Hs.423968 | ENSG00000214253 |
| | | membrane) homolog (S. | | | | |
| | | cerevisiae) | | | | |
| −2.2222222 | FKBP14 | FK506 binding protein 14, 22 kDa | 55033 | NM_017946 | Hs.390838 | ENSG00000106080 |
| −1.7241379 | FOXJ3 | forkhead box J3 | 22887 | NM_001198850 | Hs.26023 | ENSG00000198815 |
| −1.5151515 | FNBP4 | formin binding protein 4 | 23360 | NM_015308 | Hs.6834 | ENSG00000109920 |
| 1.4400000 | FMNL3 | formin-like 3 | 91010 | NM_175736 | Hs.179838 | ENSG00000161791 |
| −2.0000000 | FOXL2NB | FOXL2 neighbor | 401089 | NM_001040061 | Hs.591303 | ENSG00000206262 |
| −2.1276596 | FRY-AS1 | FRY antisense RNA 1 | 100507099 | NR_103839 | Hs.536364 | ENSG00000237637 |
| −4.0000000 | LOC642236 | FSHD region gene 1 | 642236 | NR_033907 | Hs.529357 | |
| | | pseudogene | | | | |
| −1.9230769 | FUT1 | fucosyltransferase 1 | 2523 | NM_000148 | Hs.69747 | ENSG00000174951 |
| | | (galactoside 2-alpha-L- | | | | |
| | | fucosyltransferase, H blood | | | | |
| | | group) | | | | |
| −2.4390244 | FUT2 | fucosyltransferase 2 (secretor | 2524 | NM_000511 | Hs.579928 | ENSG00000176920 |
| | | status included) | | | | |
| −2.0833333 | FUT6 | fucosyltransferase 6 (alpha | 2528 | NM_000150 | Hs.631846 | ENSG00000156413 |
| | | (1,3) fucosyltransferase) | | | | |
| −2.0408163 | FGD5P1 | FYVE, RhoGEF and PH | 100132526 | NR_036481 | Hs.637770 | ENSG00000275340 |
| | | domain containing 5 | | | | |
| | | pseudogene 1 | | | | |
| 1.5600000 | GPR108 | G protein-coupled receptor 108 | 56927 | NM_001080452 | Hs.167641 | ENSG00000125734 |
| 2.3400000 | GPR141 | G protein-coupled receptor 141 | 353345 | NM_181791 | Hs.688230 | ENSG00000187037 |
| −1.5384615 | GPR18 | G protein-coupled receptor 18 | 2841 | NM_001098200 | Hs.741589 | ENSG00000125245 |
| −2.3255814 | GPR37L1 | G protein-coupled receptor 37 | 9283 | NM_004767 | Hs.132049 | ENSG00000170075 |
| | | like 1 | | | | |
| −2.1276596 | GPR82 | G protein-coupled receptor 82 | 27197 | NM_080817 | Hs.567457 | ENSG00000171657 |
| −1.8181818 | GABPB1-AS1 | GABPB1 antisense RNA 1 | 100129387 | NR_024490 | Hs.659360 | |
| 2.4600000 | GALK1 | galactokinase 1 | 2584 | NM_000154 | Hs.407966 | ENSG00000108479 |
| −1.9607843 | GAL3ST4 | galactose-3-O- | 79690 | NM_024637 | Hs.44856 | ENSG00000197093 |
| | | sulfotransferase 4 | | | | |
| −2.5641026 | GGT8P | gamma-glutamyltransferase 8 | 645367 | NR_003503 | Hs.650223 | |
| | | pseudogene | | | | |
| −2.5000000 | GAS6-AS2 | GAS6 antisense RNA 2 (head | 100506394 | NR_044993 | Hs.132168 | ENSG00000272695 |
| | | to head) | | | | |
| −2.0833333 | GSDMA | gasdermin A | 284110 | NM_178171 | Hs.448873 | ENSG00000167914 |
| 1.7800000 | GMPPA | GDP-mannose | 29926 | NM_013335 | Hs.27059 | ENSG00000144591 |
| | | pyrophosphorylase A | | | | |
| 1.9300000 | GSN | gelsolin | 2934 | NM_000177 | Hs.522373 | ENSG00000148180 |
| −2.3809524 | GEMIN8 | gem (nuclear organelle) | 54960 | NM_001042479 | Hs.592237 | ENSG00000046647 |
| | | associated protein 8 | | | | |
| 1.7100000 | GTF2H4 | general transcription factor | 2968 | NM_001517 | Hs.485070 | ENSG00000213780 |
| | | IIH, polypeptide 4, 52 kDa | | | | |
| 1.5500000 | GTF3C2 | general transcription factor | 2976 | NM_001035521 | Hs.75782 | ENSG00000115207 |
| | | IIIC, polypeptide 2, beta | | | | |
| | | 110 kDa | | | | |
| −2.7027027 | GSG1 | germ cell associated 1 | 83445 | NM_001080554 | Hs.240053 | ENSG00000111305 |
| 1.9500000 | GHDC | GH3 domain containing | 84514 | NM_001142622 | Hs.38039 | ENSG00000167925 |
| −3.2258065 | GLIPR1L2 | GLI pathogenesis-related 1 | 144321 | NM_001270396 | Hs.406728 | ENSG00000180481 |
| | | like 2 | | | | |
| −1.5384615 | GMEB1 | glucocorticoid modulatory | 10691 | NM_006582 | Hs.632373 | ENSG00000162419 |
| | | element binding protein 1 | | | | |
| −2.2727273 | GNE | glucosamine (UDP-N-acetyl)- | 10020 | NM_001128227 | Hs.5920 | ENSG00000159921 |
| | | 2-epimerase/N- | | | | |
| | | acetylmannosamine kinase | | | | |
| 1.4600000 | GLUD1 | glutamate dehydrogenase 1 | 2746 | NM_005271 | Hs.500409 | ENSG00000148672 |
| 1.6100000 | QPCT | glutaminyl-peptide | 25797 | NM_012413 | Hs.79033 | ENSG00000115828 |
| | | cyclotransferase | | | | |
| 2.1100000 | GPX4 | glutathione peroxidase 4 | 2879 | NM_001039847 | Hs.433951 | ENSG00000167468 |
| −2.4390244 | GSTM3 | glutathione S-transferase mu | 2947 | NM_000849 | Hs.2006 | ENSG00000134202 |
| | | 3 (brain) | | | | |
| 1.9800000 | GSTP1 | glutathione S-transferase pi 1 | 2950 | NM_000852 | Hs.523836 | ENSG00000084207 |
| −1.7857143 | GK5 | glycerol kinase 5 (putative) | 256356 | NM_001039547 | Hs.135904 | ENSG00000175066 |
| −2.2727273 | GDPD1 | glycerophosphodiester | 284161 | NM_001165993 | Hs.631744 | ENSG00000153982 |
| | | phosphodiesterase domain | | | | |
| | | containing 1 | | | | |
| 1.4800000 | GYS1 | glycogen synthase 1 (muscle) | 2997 | NM_001161587 | Hs.386225 | ENSG00000104812 |
| −1.9230769 | GPLD1 | glycosylphosphatidylinositol | 2822 | NM_001503 | Hs.533291 | ENSG00000112293 |
| | | specific phospholipase D1 | | | | |
| −2.2727273 | GOSR1 | golgi SNAP receptor complex | 9527 | NM_001007024 | Hs.462680 | ENSG00000108587 |
| | | member 1 | | | | |
| −1.5384615 | GOLGA2 | golgin A2 | 2801 | NM_004486 | Hs.155827 | ENSG00000167110 |
| −2.8571429 | GOLGA6L22 | golgin A6 family-like 22 | 440243 | NM_001271664 | | ENSG00000274404 |
| 1.5600000 | GOLGA7 | golgin A7 | 51125 | NM_001002296 | Hs.654773 | ENSG00000147533 |
| −3.0303030 | GNRHR2 | gonadotropin-releasing | 114814 | NM_057163 | Hs.356873 | ENSG00000211451 |
| | | hormone (type 2) receptor 2, | | | | |
| | | pseudogene | | | | |
| −2.3809524 | GPR1-AS | GPR1 antisense RNA | 101669764 | NR_104359 | Hs.574781 | ENSG00000279220 |
| −1.6129032 | GAB2 | GRB2-associated binding | 9846 | NM_012296 | Hs.429434 | ENSG00000033327 |
| | | protein 2 | | | | |
| −2.5641026 | GRTP1 | growth hormone regulated | 79774 | NM_001286732 | Hs.745043 | ENSG00000139835 |
| | | TBC protein 1 | | | | |
| −2.1276596 | GREB1 | growth regulation by estrogen | 9687 | NM_014668 | Hs.467733 | ENSG00000196208 |
| | | in breast cancer 1 | | | | |
| 1.5400000 | GTPBP6 | GTP binding protein 6 | 8225 | NM_012227 | Hs.437145 | ENSG00000178605 |
| | | (putative) | | | | |
| −1.7543860 | GTPBP10 | GTP-binding protein 10 | 85865 | NM_001042717 | Hs.593547 | ENSG00000105793 |
| | | (putative) | | | | |
| 1.7700000 | GNB2 | guanine nucleotide binding | 2783 | NM_005273 | Hs.185172 | ENSG00000172354 |
| | | protein (G protein), beta | | | | |
| | | polypeptide 2 | | | | |
| −1.8867925 | GNG4 | guanine nucleotide binding | 2786 | NM_001098721 | Hs.159711 | ENSG00000168243 |
| | | protein (G protein), gamma 4 | | | | |
| −1.8181818 | GNL1 | guanine nucleotide binding | 2794 | NM_005275 | Hs.83147 | ENSG00000204590 |
| | | protein-like 1 | | | | |
| −1.9230769 | HIFX-AS1 | H1FX antisense RNA 1 | 339942 | NM_001025468 | Hs.450096 | |
| 1.7800000 | H2AFJ | H2A histone family, member J | 55766 | NM_018267 | Hs.524280 | ENSG00000246705 |
| 2.0200000 | H2AFX | H2A histone family, member X | 3014 | NM_002105 | Hs.477879 | ENSG00000188486 |
| 1.3500000 | HDHD3 | haloacid dehalogenase-like | 81932 | NM_001304509 | Hs.7739 | ENSG00000119431 |
| | | hydrolase domain containing 3 | | | | |
| −1.8518519 | HAUS3 | HAUS augmin-like complex, | 79441 | NM_001303143 | Hs.665869 | ENSG00000214367 |
| | | subunit 3 | | | | |
| 1.6700000 | HS1BP3 | HCLS1 binding protein 3 | 64342 | NM_022460 | Hs.531785 | ENSG00000118960 |
| −2.5641026 | HSP90AB4P | heat shock protein 90 kDa | 664618 | NR_002927 | Hs.670224 | |
| | | alpha (cytosolic), class B | | | | |
| | | member 4, pseudogene | | | | |
| 1.6900000 | HCST | hematopoietic cell signal | 10870 | NM_001007469 | Hs.117339 | ENSG00000126264 |
| | | transducer | | | | |
| −1.7543860 | HHLA2 | HERV-H LTR-associating 2 | 11148 | NM_001282556 | Hs.252351 | ENSG00000114455 |
| −2.2222222 | HHLA3 | HERV-H LTR-associating 3 | 11147 | NM_001031693 | Hs.142245 | ENSG00000197568 |
| −2.3809524 | HES2 | hes family bHLH | 54626 | NM_019089 | Hs.118727 | ENSG00000069812 |
| | | transcription factor 2 | | | | |
| −1.8181818 | HNRNPA1L2 | heterogeneous nuclear | 144983 | NM_001011724 | Hs.447506 | ENSG00000139675 |
| | | ribonucleoprotein A1-like 2 | | | | |
| 1.3800000 | H6PD | hexose-6-phosphate | 9563 | NM_001282587 | Hs.463511 | ENSG00000049239 |
| | | dehydrogenase (glucose 1- | | | | |
| | | dehydrogenase) | | | | |
| −2.4390244 | HMGB3P1 | high mobility group box 3 | 128872 | NR_002165 | Hs.558624 | |
| | | pseudogene 1 | | | | |
| 1.9100000 | HIST1H2AC | histone cluster 1, H2ac | 8334 | NM_003512 | Hs.484950 | ENSG00000180573 |
| 2.5100000 | HIST1H3H | histone cluster 1, H3h | 8357 | NM_003536 | Hs.591778 | ENSG00000278828 |
| −2.0833333 | HCG26 | HLA complex group 26 (non- | 352961 | NR_00282 | Hs.132807 | |
| | | protein coding) | | | | |
| −2.1739130 | HNF1A-AS1 | HNF1A antisense RNA 1 | 283460 | NM_178513 | Hs.612351 | ENSG00000241388 |
| 1.7400000 | HCFC1R1 | host cell factor C1 regulator 1 | 54985 | NM_001002017 | Hs.423103 | ENSG00000103145 |
| | | (XPO1 dependent) | | | | |
| −2.4390244 | HTRA4 | HtrA serine peptidase 4 | 203100 | NM_153692 | Hs.661014 | ENSG00000169495 |
| −2.0000000 | HSD17B13 | hydroxysteroid (17-beta) | 345275 | NM_001136230 | Hs.284414 | ENSG00000170509 |
| | | dehydrogenase 13 | | | | |
| −2.0833333 | HILPDA | hypoxia inducible lipid | 29923 | NM_001098786 | Hs.706124 | ENSG00000135245 |
| | | droplet-associated | | | | |
| −1.8181818 | IBA57 | IBA57, iron-sulfur cluster | 200205 | NM_001010867 | Hs.237017 | ENSG00000181873 |
| | | assembly homolog (S. | | | | |
| | | cerevisiae) | | | | |
| −2.2727273 | IKZF3 | IKAROS family zinc finger 3 | 22806 | NM_001257408 | Hs.371680 | ENSG00000161405 |
| | | (Aiolos) | | | | |
| −1.5151515 | ILF3-AS1 | ILF3 antisense RNA 1 (head | 147727 | NR_024333 | Hs.631616 | ENSG00000267100 |
| | | to head) | | | | |
| −1.9230769 | IRGQ | immunity-related GTPase | 126298 | NM_001007561 | Hs.6217 | ENSG00000167378 |
| | | family, Q | | | | |
| −1.9230769 | IPO5P1 | importin 5 pseudogene 1 | 100132815 | NR_103741 | Hs.629249 | ENSG00000269837 |
| −2.3255814 | INE1 | inactivation escape 1 (non- | 8552 | NM_003669 | Hs.657350 | ENSG00000224975 |
| | | protein coding) | | | | |
| −2.4390244 | IDO1 | indoleamine 2,3-dioxygenase 1 | 3620 | NM_002164 | Hs.840 | ENSG00000131203 |
| −2.2727273 | INMT | indolethylamine N- | 11185 | NM_001199219 | Hs.632629 | ENSG00000241644 |
| | | methyltransferase | | | | |
| −1.9607843 | INHBA-AS1 | INHBA antisense RNA 1 | 285954 | NR_027118 | Hs.656869 | ENSG00000224116 |
| −1.5384615 | ING5 | inhibitor of growth family, | 84289 | NM_032329 | Hs.529172 | ENSG00000168395 |
| | | member 5 | | | | |
| −2.5641026 | INGX | inhibitor of growth family, X- | 27160 | NR_002226 | Hs.721806 | ENSG00000243468 |
| | | linked, pseudogene | | | | |
| 1.3800000 | INPP5E | inositol polyphosphate-5- | 56623 | NM_019892 | Hs.120998 | ENSG00000148384 |
| | | phosphatase, 72 kDa | | | | |
| 1.6500000 | INTS9 | integrator complex subunit 9 | 55756 | NM_001145159 | Hs.162397 | ENSG00000104299 |
| −1.8867925 | ITIH5 | inter-alpha-trypsin inhibitor | 80760 | NM_001001851 | Hs.498586 | ENSG00000123243 |
| | | heavy chain family, member 5 | | | | |
| 1.7000000 | IRF2BP1 | interferon regulatory factor 2 | 26145 | NM_015649 | Hs.515477 | ENSG00000170604 |
| | | binding protein 1 | | | | |
| −2.3809524 | IFNLR1 | interferon, lambda receptor 1 | 163702 | NM_170743 | Hs.221375 | ENSG00000185436 |
| −2.0000000 | IL17RD | interleukin 17 receptor D | 54756 | NM_017563 | Hs.150725 | ENSG00000144730 |
| −1.5625000 | ITSN2 | intersectin 2 | 50618 | NM_006277 | Hs.432562. | ENSG00000198399 |
| −1.9607843 | IFT22 | intraflagellar transport 22 | 64792 | NM_001130820 | Hs.389104 | ENSG00000128581 |
| −2.2222222 | IAPP | islet amyloid polypeptide | 3375 | NM_000415 | Hs.46835 | ENSG00000121351 |
| −2.0000000 | ICA1L | islet cell autoantigen | 130026 | NM_001288622 | Hs.516629 | ENSG00000163596 |
| | | 1, 69 kDa-like | | | | |
| 2.3900000 | JOSD2 | Josephin domain containing 2 | 126119 | NM_001270639 | Hs.467151 | ENSG00000161677 |
| −2.2222222 | JPX | JPX transcript, XIST | 554203 | NR_024582 | Hs.648316 | ENSG00000225470 |
| | | activator (non-protein coding) | | | | |
| 1.6100000 | JTB | jumping translocation | 10899 | NM_006694 | Hs.6396 | ENSG00000143543 |
| | | breakpoint | | | | |
| −2.6315789 | KCNQ1OT1 | KCNQ1 opposite | 10984 | NR_002728 | Hs.604823 | ENSG00000269821 |
| | | strand/antisense transcript 1 | | | | |
| | | (non-protein coding) | | | | |
| −2.2727273 | KDELC2 | KDEL (Lys-Asp-Glu-Leu) | 143888 | NM_153705 | Hs.83286 | ENSG00000178202 |
| | | (SEQ ID NO: 802) containing 2 | | | | |
| 1.9100000 | KDELR1 | KDEL (Lys-Asp-Glu-Leu) | 10945 | NM_006801 | Hs.515515 | ENSG00000105438 |
| | | (SEQ ID NO: 802) | | | | |
| | | endoplasmic reticulum | | | | |
| | | protein retention receptor 1 | | | | |
| 2.0800000 | KDELR2 | KDEL (Lys-Asp-Glu-Leu) | 11014 | NM_001100603 | Hs.654552 | ENSG00000136240 |
| | | (SEQ ID NO: 802) | | | | |
| | | endoplasmic reticulum | | | | |
| | | protein retention receptor 2 | | | | |
| −2.5000000 | KDM4A-AS1 | KDM4A antisense RNA 1 | 100132774 | NR_033827 | Hs.655569 | |
| 1.7100000 | KLHDC3 | kelch domain containing 3 | 116138 | NM_001242872 | Hs.412468 | ENSG00000124702 |
| −2.2222222 | KBTBD12 | kelch repeat and BTB (POZ) | 166348 | NM_207335 | Hs.132087 | ENSG00000187715 |
| | | domain containing 12 | | | | |
| −1.8181818 | KRT8 | keratin 8, type II | 3856 | NM_001256282 | Hs.533782 | ENSG00000170421 |
| −3.4482759 | KIAA0101 | KIAA0101 | 9768 | NM_001029989 | Hs.81892 | ENSG00000166803 |
| −2.8571429 | KIAA1161 | KIAA1161 | 57462 | NM_020702 | Hs.522083 | ENSG00000164976 |
| −2.2222222 | KIAA1456 | KIAA1456 | 57604 | NM_001099677 | Hs.202521 | ENSG00000250305 |
| −2.1739130 | KIAA1614 | KIAA1614 | 57710 | NM_020950 | Hs.734816 | ENSG00000135835 |
| −2.0000000 | KIAA1919 | KIAA1919 | 91749 | NM_153369 | Hs.400572 | ENSG00000173214 |
| −2.3255814 | KIR3DX1 | killer cell immunoglobulin- | 90011 | NM_001047605 | Hs.288520 | ENSG00000104970 |
| | | like receptor, three domains, X1 | | | | |
| −3.2258065 | KLRD1 | killer cell lectin-like receptor | 3824 | NM_001114396 | Hs.562457 | ENSG00000134539 |
| | | subfamily D, member 1 | | | | |
| −1.5625000 | KIN | Kin17 DNA and RNA | 22944 | NM_012311 | Hs.397918 | ENSG00000151657 |
| | | binding protein | | | | |
| −2.2727273 | KIF18B | kinesin family member 18B | 146909 | NM_001080443 | Hs.135094 | ENSG00000186185 |
| −1.3157895 | KIF1B | kinesin family member 1B | 23095 | NM_015074 | Hs.97858 | ENSG00000054523 |
| −2.2222222 | KIF3A | kinesin family member 3A | 11127 | NM_001300791 | Hs.43670 | ENSG00000131437 |
| −2.8571429 | L2HGDH | L-2-hydroxyglutarate | 79944 | NM_024884 | Hs.256034 | ENSG00000087299 |
| | | dehydrogenase | | | | |
| −2.0408163 | LARS2-AS1 | LARS2 antisense RNA 1 | 100885795 | NR_048543 | Hs.641094 | ENSG00000232455 |
| −2.2727273 | LGMN | legumain | 5641 | NM_001008530 | Hs.18069 | ENSG00000100600 |
| −2.0000000 | LRRC2 | leucine rich repeat containing 2 | 79442 | NM_024512 | Hs.657345 | ENSG00000163827 |
| −1.9607843 | LRRC27 | leucine rich repeat containing 27 | 80313 | NM_001143757 | Hs.119897 | ENSG00000148814 |
| −1.3698630 | LRRC37BP1 | leucine rich repeat containing | 147172 | NM_207323 | Hs.568209 | |
| | | 37B pseudogene 1 | | | | |
| −2.1739130 | LRTOMT | leucine rich transmembrane | 220074 | NM_001145307 | Hs.317243 | ENSG00000184154 |
| | | and O-methyltransferase | | | | |
| | | domain containing | | | | |
| 1.5400000 | LILRA2 | leukocyte immunoglobulin- | 11027 | NM_001130917 | Hs.655593 | ENSG00000239998 |
| | | like receptor, subfamily A | | | | |
| | | (with TM domain), member 2 | | | | |
| −2.9411765 | LIFR-AS1 | LIFR antisense RNA 1 | 100506495 | NR_103553 | Hs.657602 | ENSG00000244968 |
| 2.1200000 | LIMD2 | LIM domain containing 2 | 80774 | NM_030576 | Hs.591166 | ENSG00000136490 |
| −2.0408163 | LMX1B | LIM homeobox transcription | 4010 | NM_001174146 | Hs.129133 | ENSG00000136944 |
| | | factor 1, beta | | | | |
| −2.3255814 | LITD1 | LINE-1 type transposase | 54596 | NM_001164835 | Hs.685462 | ENSG00000240563 |
| | | domain containing 1 | | | | |
| −1.9230769 | LINC01010 | long intergenic non-protein | 154092 | NR_038216 | Hs.223718 | ENSG00000236700 |
| | | coding RNA 1010 | | | | |
| −2.1739130 | LINC01012 | long intergenic non-protein | 100507173 | NR_038292 | Hs.635987 | |
| | | coding RNA 1012 | | | | |
| −2.0833333 | LINC01016 | long intergenic non-protein | 100507584 | NR_038989 | Hs.547104 | ENSG00000249346 |
| | | coding RNA 1016 | | | | |
| −2.7027027 | LINC01057 | long intergenic non-protein | 101928079 | NR_104131 | Hs.596857 | ENSG00000224081 |
| | | coding RNA 1057 | | | | |
| −2.2727273 | LINC01087 | long intergenic non-protein | 101927994 | NR_108087 | Hs.635757 | ENSG00000224559 |
| | | coding RNA 1087 | | | | |
| −2.3255814 | LINC01205 | long intergenic non-protein | 401082 | NM_001145553 | Hs.477089 | ENSG00000228980 |
| | | coding RNA 1205 | | | | |
| −2.2222222 | LINC01207 | long intergenic non-protein | 100505989 | NR_038834 | Hs.328236 | ENSG00000248771 |
| | | coding RNA 1207 | | | | |
| −2.3255814 | LINC01209 | long intergenic non-protein | 101928684 | NR_110819 | Hs.639352 | ENSG00000228308 |
| | | coding RNA 1209 | | | | |
| −2.2222222 | LINC01226 | long intergenic non-protein | 284551 | NR_027085 | Hs.658659 | ENSG00000223907 |
| | | coding RNA 1226 | | | | |
| −2.1739130 | LINC01239 | long intergenic non-protein | 441389 | NR_038977 | | ENSG00000234840 |
| | | coding RNA 1239 | | | | |
| −2.1276596 | LINC01247 | long intergenic non-protein | 101929390 | NR_110251 | Hs.434407 | ENSG00000227007 |
| | | coding RNA 1247 | | | | |
| −1.8867925 | LINC01252 | long intergenic non-protein | 338817 | NR_033890 | Hs.733066 | ENSG00000247157 |
| | | coding RNA 1252 | | | | |
| −1.9607843 | LINC01299 | long intergenic non-protein | 286186 | NR_033893 | Hs.449427 | ENSG00000254081 |
| | | coding RNA 1299 | | | | |
| −2.3809524 | LINC01356 | long intergenic non-protein | 100996702 | NR_103746 | Hs.632431 | ENSG00000215866 |
| | | coding RNA 1356 | | | | |
| −2.1739130 | LINC00294 | long intergenic non-protein | 283267 | NR_015451 | Hs.533701 | |
| | | coding RNA 294 | | | | |
| −1.8181818 | LINC00330 | long intergenic non-protein | 144817 | NR_038433 | Hs.585616 | ENSG00000235097 |
| | | coding RNA 330 | | | | |
| −1.8518519 | LINC00342 | long intergenic non-protein | 150759 | NR_103734 | Hs.503463 | |
| | | coding RNA 342 | | | | |
| −2.3255814 | LINC00346 | long intergenic non-protein | 283487 | NM_178514 | Hs.245390 | ENSG00000255874 |
| | | coding RNA 346 | | | | |
| −2.7027027 | LINC00381 | long intergenic non-protein | 100874151 | NR_047005 | Hs.564552 | ENSG00000226240 |
| | | coding RNA 381 | | | | |
| −2.1739130 | LINC00410 | long intergenic non-protein | 144776 | NR_027039 | Hs.434120 | ENSG00000231674 |
| | | coding RNA 410 | | | | |
| −3.0303030 | LINC00458 | long intergenic non-protein | 100507428 | NR_108062 | Hs.351262 | ENSG00000234787 |
| | | coding RNA 458 | | | | |
| −2.0000000 | LINC00470 | long intergenic non-protein | 56651 | NM_031416 | Hs.541165 | |
| | | coding RNA 470 | | | | |
| −2.5641026 | LINC00485 | long intergenic non-protein | 283432 | NR_033855 | Hs.382110 | ENSG00000258169 |
| | | coding RNA 485 | | | | |
| −3.5714286 | LINC00506 | long intergenic non-protein | 100846978 | NR_047469 | Hs.570649 | |
| | | coding RNA 506 | | | | |
| −3.2258065 | LINC00507 | long intergenic non-protein | 100862680 | NR_046392 | Hs.385496 | ENSG00000256193 |
| | | coding RNA 507 | | | | |
| −2.5000000 | LINC00547 | long intergenic non-protein | 400121 | NR_040244 | Hs.558894 | ENSG00000275226 |
| | | coding RNA 547 | | | | |
| −2.3809524 | LINC00620 | long intergenic non-protein | 285375 | NR_027103 | Hs.319969 | ENSG00000224514 |
| | | coding RNA 620 | | | | |
| −2.4390244 | LINC00649 | long intergenic non-protein | 100506334 | NR_038883 | Hs.532855 | ENSG00000237945 |
| | | coding RNA 649 | | | | |
| −2.5641026 | LINC00652 | long intergenic non-protein | 29075 | NM_014162 | Hs.584899 | ENSG00000179935 |
| | | coding RNA 652 | | | | |
| −2.7777778 | LINC00670 | long intergenic non-protein | 284034 | NR_034144 | Hs.376614 | ENSG00000179136 |
| | | coding RNA 670 | | | | |
| −2.3809524 | LINC00672 | long intergenic non-protein | 100505576 | NR_038847 | Hs.634043 | ENSG00000263874 |
| | | coding RNA 672 | | | | |
| −3.8461538 | LINC00678 | long intergenic non-protein | 101410541 | NR_102708 | Hs.471439 | ENSG00000254934 |
| | | coding RNA 678 | | | | |
| −2.0833333 | LINC00889 | long intergenic non-protein | 158696 | NR_026935 | Hs.558664 | |
| | | coding RNA 889 | | | | |
| −1.9607843 | LINC00907 | long intergenic non-protein | 284260 | NR_046174 | Hs.652819 | ENSG00000267586 |
| | | coding RNA 907 | | | | |
| −2.1739130 | LINC00924 | long intergenic non-protein | 145820 | NR_027132 | Hs.652702 | ENSG00000259134 |
| | | coding RNA 924 | | | | |
| −2.7027027 | LINC00958 | long intergenic non-protein | 100506305 | NR_038904 | Hs.153408 | ENSG00000251381 |
| | | coding RNA 958 | | | | |
| −2.8571429 | LINC00965 | long intergenic non-protein | 349196 | NM_001025473 | Hs.559040 | |
| | | coding RNA 965 | | | | |
| −2.7027027 | LINC00970 | long intergenic non-protein | 101978719 | NR_104091 | Hs.517849 | ENSG00000203601 |
| | | coding RNA 970 | | | | |
| −2.5641026 | LOH12CR2 | loss of heterozygosity, 12, | 503693 | NR_024061 | Hs.67553 | ENSG00000205791 |
| | | chromosomal region 2 (non- | | | | |
| | | protein coding) | | | | |
| 1.9300000 | LRP10 | low density lipoprotein | 26020 | NM_014045 | Hs.525232 | ENSG00000197324 |
| | | receptor-related protein 10 | | | | |
| −2.0408163 | LRRN4CL | LRRN4 C-terminal like | 221091 | NM_203422 | Hs.427449 | ENSG00000177363 |
| −1.6949153 | LUC7L2 | LUC7-like 2 (S. cerevisiae) | 51631 | NM_001244585 | Hs.731488 | ENSG00000146963 |
| 1.9500000 | LYL1 | lymphoblastic leukemia | 4066 | NM_005583 | Hs.46446 | ENSG00000104903 |
| | | associated hematopoiesis | | | | |
| | | regulator 1 | | | | |
| −1.8518519 | LYRM7 | LYR motif containing 7 | 90624 | NM_001293735 | Hs.115467 | ENSG00000186687 |
| 2.1100000 | LYPLA2 | lysophospholipase II | 11313 | NM_007260 | Hs.533479 | ENSG00000011009 |
| −2.7777778 | MAB21L3 | mab-21-like 3 (C. elegans) | 126868 | NM_152367 | Hs.376194 | ENSG00000173212 |
| −2.0000000 | MRO | maestro | 83876 | NM_001127174 | Hs.30495 | ENSG00000134042 |
| 2.1000000 | MDP1 | magnesium-dependent | 145553 | NM_001199821 | Hs.220963 | ENSG00000213920 |
| | | phosphatase 1 | | | | |
| 1.8200000 | MDH1 | malate dehydrogenase 1, | 4190 | NM_001199111 | Hs.526521 | ENSG00000014641 |
| | | NAD (soluble) | | | | |
| 1.9200000 | MDH2 | malate dehydrogenase 2, | 4191 | NM_001282403 | Hs.520967 | ENSG00000146701 |
| | | NAD (mitochondrial) | | | | |
| −1.4492754 | MCTS1 | malignant T cell amplified | 28985 | NM_001137554 | Hs.102696 | ENSG00000232119 |
| | | sequence 1 | | | | |
| −2.1276596 | MAN1B1-AS1 | MAN1B1 antisense RNA 1 | 100289341 | NR_027447 | Hs.593896 | ENSG00000268996 |
| | | (head to head) | | | | |
| −3.8461538 | MANEAL | mannosidase, endo-alpha-like | 149175 | NM_001031740 | Hs.534562 | ENSG00000185090 |
| −1.8181818 | MAP7D3 | MAP7 domain containing 3 | 79649 | NM_001173516 | Hs.446275 | ENSG00000129680 |
| −2.1276596 | MRGPRX3 | MAS-related GPR, member X3 | 117195 | NM_054031 | Hs.380177 | ENSG00000179826 |
| −1.5151515 | MDM4 | MDM4, p53 regulator | 4194 | NM_001204171 | Hs.497492 | ENSG00000198625 |
| −2.1739130 | MED15P9 | mediator complex subunit 15 | 285103 | NR_033903 | Hs.570106 | ENSG00000223760 |
| | | pseudogene 9 | | | | |
| −2.3255814 | MED18 | mediator complex subunit 18 | 54797 | NM_001127350 | Hs.479911 | ENSG00000130772 |
| −1.3888889 | MED29 | mediator complex subunit 29 | 55588 | NM_017592 | Hs.611541 | ENSG00000063322 |
| −2.0408163 | MLANA | melan-A | 2315 | NM_005511 | Hs.154069 | ENSG00000120215 |
| −2.0833333 | MAGEA10 | melanoma antigen family A10 | 4109 | NM_001011543 | Hs.18048 | ENSG00000124260 |
| −1.9230769 | MREG | melanoregulin | 55686 | NM_018000 | Hs.620391 | ENSG00000118242 |
| −2.0833333 | MS4A10 | membrane-spanning 4- | 341116 | NM_206893 | Hs.591956 | ENSG00000172689 |
| | | domains, subfamily A, | | | | |
| | | member 10 | | | | |
| 1.7600000 | METTL17 | methyltransferase like 17 | 64745 | NM_001029991 | Hs.512693 | ENSG00000165792 |
| −2.0408163 | METTL20 | methyltransferase like 20 | 254013 | NM_001135863 | Hs.740628 | ENSG00000139160 |
| −2.5000000 | METTL21A | methyltransferase like 21A | 151194 | NM_001127395 | Hs.664764 | ENSG00000144401 |
| 1.7000000 | METTL23 | methyltransferase like 23 | 124512 | NM_001080510 | Hs.74655 | ENSG00000181038 |
| −2.1739130 | METTL2A | methyltransferase like 2A | 339175 | NM_001005372 | Hs.381204 | ENSG00000087995 |
| −1.9607843 | METTL2B | methyltransferase like 2B | 55798 | NM_018396 | Hs.433213 | ENSG00000165055 |
| 1.3000000 | METTL4 | methyltransferase like 4 | 64863 | NM_022840 | Hs.126888 | ENSG00000101574 |
| −2.0408163 | METTL8 | methyltransferase like 8 | 79828 | NM_024770 | Hs.135146 | ENSG00000123600 |
| 1.7900000 | MFNG | MFNG O-fucosylpeptide 3- | 4242 | NM_001166343 | Hs.517603 | ENSG00000100060 |
| | | beta-N- | | | | |
| | | acetylglucosaminyltransferase | | | | |
| 6.0100000 | MIR3655 | microRNA 3655 | 100500820 | NR_037428 | | ENSG00000264052 |
| −6.6666667 | MIR5194 | microRNA 5194 | 100847051 | NR_049826 | | ENSG00000264653 |
| 3.3700000 | MIR564 | microRNA 564 | 693149 | NR_030290 | | ENSG00000207783 |
| 2.8700000 | MIR636 | microRNA 636 | 693221 | NR_030366 | | ENSG00000207556 |
| 9.2400000 | MIR7845 | microRNA 7845 | 102465835 | NR_106999 | | ENSG00000277590 |
| 1.7900000 | MGST1 | microsomal glutathione S- | 4257 | NM_001260511 | Hs.389700 | ENSG00000008394 |
| | | transferase 1 | | | | |
| −1.7241379 | MICAL3 | microtubule associated | 57553 | NM_001122731 | Hs.528024 | ENSG00000243156 |
| | | monooxygenase, calponin | | | | |
| | | and LIM domain containing 3 | | | | |
| −2.7027027 | MAPILC3C | microtubule-associated | 440738 | NM_001004343 | Hs.534971 | ENSG00000197769 |
| | | protein 1 light chain 3 gamma | | | | |
| 1.7000000 | MCMBP | minichromosome | 79892 | NM_001256378 | Hs.124246 | ENSG00000197771 |
| | | maintenance complex binding | | | | |
| | | protein | | | | |
| −1.7857143 | MIR210HG | MIR210 host gene | 100506211 | NR_038262 | Hs.446388 | ENSG00000247095 |
| −1.8181818 | MIRLET7BHG | MIRLET7B host gene | 400931 | NM_207477 | Hs.235838 | ENSG00000197182 |
| −2.4390244 | MTFMT | mitochondrial methionyl- | 123263 | NM_139242 | Hs.531615 | ENSG00000103707 |
| | | tRNA formyltransferase | | | | |
| 1.7600000 | MRPL12 | mitochondrial ribosomal | 6182 | NM_002949 | Hs.109059 | ENSG00000262814 |
| | | protein L12 | | | | |
| 2.0900000 | MRPL34 | mitochondrial ribosomal | 64981 | NM_023937 | Hs.515242 | ENSG00000130312 |
| | | protein L34 | | | | |
| 1.4900000 | MRPL37 | mitochondrial ribosomal | 51253 | NM_016491 | Hs.584908 | ENSG00000116221 |
| | | protein L37 | | | | |
| 1.8000000 | MRPL38 | mitochondrial ribosomal | 64978 | NM_032478 | Hs.442609 | ENSG00000204316 |
| | | protein L38 | | | | |
| 1.7300000 | MRPL41 | mitochondrial ribosomal | 64975 | NM_032477 | Hs.44017 | ENSG00000182154 |
| | | protein L41 | | | | |
| 2.0000000 | MRPS12 | mitochondrial ribosomal | 6183 | NM_021107 | Hs.411125 | ENSG00000128626 |
| | | protein S12 | | | | |
| −1.8518519 | MAPK13 | mitogen-activated protein | 5603 | NM_002754 | Hs.178695 | ENSG00000156711 |
| | | kinase 13 | | | | |
| 1.9300000 | MAP2K2 | mitogen-activated protein | 5605 | NM_030662 | Hs.465627 | ENSG00000126934 |
| | | kinase kinase 2 | | | | |
| 1.5600000 | MAP2K5 | mitogen-activated protein | 5607 | NM_001206804 | Hs.114198 | ENSG00000137764 |
| | | kinase kinase 5 | | | | |
| −1.6949153 | MAP3K9 | mitogen-activated protein | 4293 | NM_001284230 | Hs.445496 | ENSG00000006432 |
| | | kinase kinase kinase 9 | | | | |
| 1.8800000 | MZT2A | mitotic spindle organizing | 653784 | NM_001085365 | Hs.655067 | ENSG00000173272 |
| | | protein 2A | | | | |
| 1.4700000 | MLX | MLX, MAX dimerization | 6945 | NM_170607 | Hs.383019 | ENSG00000108788 |
| | | protein | | | | |
| −2.5000000 | MOCS3 | molybdenum cofactor | 27304 | NM_014484 | Hs.159410 | ENSG00000124217 |
| | | synthesis 3 | | | | |
| −2.1739130 | MMD2 | monocyte to macrophage | 221938 | NM_001100600 | Hs.558694 | ENSG00000136297 |
| | | differentiation-associated 2 | | | | |
| −1.9230769 | MORN4 | MORN repeat containing 4 | 118812 | NM_001098831 | Hs.217409 | ENSG00000171160 |
| −1.9607843 | MPV17L | MPV17 mitochondrial | 255027 | NM_001128423 | Hs.720673 | ENSG00000156968 |
| | | membrane protein-like | | | | |
| −2.2222222 | MUC6 | mucin 6, oligomeric | 4588 | NM_005961 | Hs.528432 | ENSG00000184956 |
| | | mucus/gel-forming | | | | |
| −2.3255814 | MMRN2 | multimerin 2 | 79812 | NM_024756 | Hs.524479 | ENSG00000173269 |
| −1.7857143 | MCFD2 | multiple coagulation factor | 90411 | NM_001171506 | Hs.662152 | ENSG00000180398 |
| | | deficiency 2 | | | | |
| 1.4700000 | MUS81 | MUS81 structure-specific | 80198 | NM_025128 | Hs.288798 | ENSG00000172732 |
| | | endonuclease subunit | | | | |
| 2.0500000 | MYPOP | Myb-related transcription | 339344 | NM_001012643 | Hs.515478 | ENSG00000176182 |
| | | factor, partner of profilin | | | | |
| 1.3800000 | MBP | myelin basic protein | 4155 | NM_001025081 | Hs.551713 | ENSG00000197971 |
| −1.7857143 | MYEF2 | myelin expression factor 2 | 50804 | NM_001301210 | Hs.6638 | ENSG00000104177 |
| −2.5000000 | MOG | myelin oligodendrocyte | 4340 | NM_001008228 | Hs.141308 | ENSG00000204655 |
| | | glycoprotein | | | | |
| −1.8181818 | MPZL3 | myelin protein zero-like 3 | 196264 | NM_001286152 | Hs.15396 | ENSG00000160588 |
| −1.8867925 | MYLK3 | myosin light chain kinase 3 | 91807 | NM_182493 | Hs.130465 | ENSG00000140795 |
| 1.4000000 | MYO7B | myosin VIIB | 4648 | NM_001080527 | Hs.154578 | ENSG00000169994 |
| −1.6666667 | NAA16 | N(alpha)-acetyltransferase 16, | 79612 | NM_001110798 | Hs.512914 | ENSG00000172766 |
| | | NatA auxiliary subunit | | | | |
| 1.6400000 | NAGA | N-acetylgalactosaminidase, | 4668 | NM_000262 | Hs.75372 | ENSG00000198951 |
| | | alpha- | | | | |
| −2.0833333 | NWD1 | NACHT and WD repeat | 284434 | NM_001007525 | Hs.406014 | ENSG00000188039 |
| | | domain containing 1 | | | | |
| −1.8867925 | NQO1 | NAD(P)H dehydrogenase, | 1728 | NM_000903 | Hs.406515 | ENSG00000181019 |
| | | quinone 1 | | | | |
| 1.8100000 | NDUFA7 | NADH dehydrogenase | 4701 | NM_005001 | Hs.333427 | ENSG00000267855 |
| | | (ubiquinone) 1 alpha | | | | |
| | | subcomplex, 7, 14.5 kDa | | | | |
| 1.9000000 | NDUFB11 | NADH dehydrogenase | 54539 | NM_001135998 | Hs.521969 | ENSG00000147123 |
| | | (ubiquinone) 1 beta | | | | |
| | | subcomplex, 11, 17.3 kDa | | | | |
| 1.7300000 | NDUFB6 | NADH dehydrogenase | 4712 | NM_001199987 | Hs.493668 | ENSG00000165264 |
| | | (ubiquinone) 1 beta | | | | |
| | | subcomplex, 6, 17 kDa | | | | |
| 1.5100000 | NDUFB7 | NADH dehydrogenase | 4713 | NM_004146 | Hs.532853 | ENSG00000099795 |
| | | (ubiquinone) 1 beta | | | | |
| | | subcomplex, 7, 18 kDa | | | | |
| 1.3800000 | NDUFB8 | NADH dehydrogenase | 4714 | NM_001284367 | Hs.523215 | ENSG00000166136 |
| | | (ubiquinone) 1 beta | | | | |
| | | subcomplex, 8, 19 kDa | | | | |
| 2.0600000 | NDUFC1 | NADH dehydrogenase | 4717 | NM_001184986 | Hs.84549 | ENSG00000109390 |
| | | (ubiquinone) 1, subcomplex | | | | |
| | | unknown, 1, 6 kDa | | | | |
| −1.7857143 | NDUFAF7 | NADH dehydrogenase | 55471 | NM_001083946 | Hs.433466 | ENSG00000003509 |
| | | (ubiquinone) complex I, | | | | |
| | | assembly factor 7 | | | | |
| 1.8500000 | NDUFS7 | NADH dehydrogenase | 374291 | NM_024407 | Hs.211914 | ENSG00000115286 |
| | | (ubiquinone) Fe—S protein 7, | | | | |
| | | 20 kDa (NADH-coenzyme Q | | | | |
| | | reductase) | | | | |
| −1.4492754 | NECAP1 | NECAP endocytosis | 25977 | NM_015509 | Hs.555927 | ENSG00000089818 |
| | | associated 1 | | | | |
| −1.9607843 | NPHS1 | nephrosis 1, congenital, | 4868 | NM_004646 | Hs.122186 | ENSG00000161270 |
| | | Finnish type (nephrin) | | | | |
| −2.6315789 | NBPF20 | neuroblastoma breakpoint | 100288142 | NM_001278267 | Hs.445080 | ENSG00000162825 |
| | | family, member 20 | | | | |
| −2.6315789 | NEXN-AS1 | NEXN antisense RNA 1 | 374987 | NM_001039463 | Hs.632414 | ENSG00000235927 |
| 1.5200000 | NKIRAS2 | NFKB inhibitor interacting | 28511 | NM_001001349 | Hs.632252 | ENSG00000168256 |
| | | Ras-like 2 | | | | |
| −1.9230769 | NMNAT1 | nicotinamide nucleotide | 64802 | NM_001297778 | Hs.633762 | ENSG00000173614 |
| | | adenylyltransferase 1 | | | | |
| −2.2222222 | NEK2 | NIMA-related kinase 2 | 4751 | NM_001204182 | Hs.153704 | ENSG00000117650 |
| 1.7900000 | NIPAL2 | NIPA-like domain containing 2 | 79815 | NM_024759 | Hs.309489 | ENSG00000104361 |
| 1.6200000 | NIPSNAP3A | nipsnap homolog 3A (C. | 25934 | NM_015469 | Hs.530275 | ENSG00000136783 |
| | | elegans) | | | | |
| −1.8518519 | NMT2 | N-myristoyltransferase 2 | 9397 | NM_004808 | Hs.60339 | ENSG00000152465 |
| −1.9607843 | NCMAP | noncompact myelin | 400746 | NM_001010980 | Hs.200253 | ENSG00000184454 |
| | | associated protein | | | | |
| −1.9230769 | NFKBIZ | nuclear factor of kappa light | 64332 | NM_001005474 | Hs.319171 | ENSG00000144802 |
| | | polypeptide gene enhancer in | | | | |
| | | B-cells inhibitor, zeta | | | | |
| −1.8518519 | NUGGC | nuclear GTPase, germinal | 389643 | NM_001010906 | Hs.370129 | ENSG00000189233 |
| | | center associated | | | | |
| −1.8867925 | NPAP1 | nuclear pore associated | 23742 | NM_018958 | Hs.649663 | ENSG00000185823 |
| | | protein 1 | | | | |
| 2.0700000 | NPIPA2 | nuclear pore complex | 642799 | NM_001277324 | | ENSG00000254852 |
| | | interacting protein family, | | | | |
| | | member A2 | | | | |
| −2.0408163 | NRIP3 | nuclear receptor interacting | 56675 | NM_020645 | Hs.523467 | ENSG00000175352 |
| | | protein 3 | | | | |
| −1.4705882 | NR6A1 | nuclear receptor subfamily 6, | 2649 | NM_001278546 | Hs.20131 | ENSG00000148200 |
| | | group A, member 1 | | | | |
| −1.8518519 | NFX1 | nuclear transcription factor, | 4799 | NM_002504 | Hs.413074 | ENSG00000086102 |
| | | X-box binding 1 | | | | |
| 1.2500000 | NUP133 | nucleoporin 133 kDa | 55746 | NM_018230 | Hs.12457 | ENSG00000069248 |
| −2.6315789 | NXN | nucleoredoxin | 64359 | NM_001205319 | Hs.527989 | ENSG00000167693 |
| −2.3255814 | NXNL2 | nucleoredoxin-like 2 | 158046 | NM_001161625 | Hs.734507 | ENSG00000130045 |
| 1.8700000 | NUDT22 | nudix (nucleoside | 84304 | NM_001128612 | Hs.656074 | ENSG00000149761 |
| | | diphosphate linked moiety | | | | |
| | | X)-type motif 22 | | | | |
| 1.9300000 | OCEL1 | occludin/ELL domain | 79629 | NM_024578 | Hs.422676 | ENSG00000099330 |
| | | containing 1 | | | | |
| −2.0408163 | OLAH | oleoy1-ACP hydrolase | 55301 | NM_001039702 | Hs.24309 | ENSG00000152463 |
| −2.0833333 | OR11A1 | olfactory receptor, family 11, | 26531 | NM_013937 | Hs.676010 | ENSG00000204694 |
| | | subfamily A, member 1 | | | | |
| 2.0400000 | OLIG1 | oligodendrocyte transcription | 116448 | NM_138983 | Hs.56663 | ENSG00000184221 |
| | | factor 1 | | | | |
| −2.5000000 | OPHN1 | oligophrenin 1 | 4983 | NM_002547 | Hs.128824 | ENSG00000079482 |
| −1.8518519 | OSMR | oncostatin M receptor | 9180 | NM_001168355 | Hs.120658 | ENSG00000145623 |
| 1.8100000 | OPN3 | opsin 3 | 23596 | NM_001030011 | Hs.409081 | ENSG00000054277 |
| −1.6949153 | OPA3 | optic atrophy 3 (autosomal | 80207 | NM_001017989 | Hs.466945 | ENSG00000125741 |
| | | recessive, with chorea and | | | | |
| | | spastic paraplegia) | | | | |
| 1.8600000 | ORAI3 | ORAI calcium release- | 93129 | NM_152288 | Hs.745104 | ENSG00000175938 |
| | | activated calcium modulator 3 | | | | |
| −2.8571429 | ORC4 | origin recognition complex, | 5000 | NM_001190879 | Hs.558364 | ENSG00000115947 |
| | | subunit 4 | | | | |
| −1.9230769 | OSGEPL1-AS1 | OSGEPL1 antisense RNA 1 | 101409258 | NR_102429 | Hs.738558 | ENSG00000253559 |
| −1.8518519 | OTUD3 | OTU deubiquitinase 3 | 23252 | NM_015207 | Hs.374987 | ENSG00000169914 |
| −2.8571429 | OTUD6A | OTU deubiquitinase 6A | 139562 | NM_207320 | Hs.447381 | ENSG00000189401 |
| −3.2258065 | ODF2L | outer dense fiber of sperm | 57489 | NM_001007022 | Hs.149360 | ENSG00000122417 |
| | | tails 2-like | | | | |
| −1.6666667 | OSBPL2 | oxysterol binding protein-like 2 | 9885 | NM_001001691 | Hs.473254 | ENSG00000130703 |
| 1.5200000 | OSBPL7 | oxysterol binding protein-like 7 | 114881 | NM_017731 | Hs.463320 | ENSG00000006025 |
| −2.5000000 | LOC646214 | p21 protein (Cdc42/Rac)- | 646214 | NR_027053 | Hs.510697 | ENSG00000278673 |
| | | activated kinase 2 | | | | |
| | | pseudogene | | | | |
| −2.1739130 | P2RX5- | P2RX5-TAX1BP3 | 100533970 | NR_037928 | Hs.731607 | |
| | TAX1BP3 | readthrough (NMD | | | | |
| | | candidate) | | | | |
| −1.6393443 | PAF1 | Paf1, RNA polymerase II | 54623 | NM_001256826 | Hs.466714 | ENSG00000006712 |
| | | associated factor, homolog | | | | |
| | | (S. cerevisiae) | | | | |
| −2.5000000 | PTCSC3 | papillary thyroid carcinoma | 100886964 | NR_049735 | Hs.742592 | ENSG00000259104 |
| | | susceptibility candidate 3 | | | | |
| | | (non-protein coding) | | | | |
| −2.5000000 | PARD6G | par-6 family cell polarity | 84552 | NM_032510 | Hs.654920 | ENSG00000178184 |
| | | regulator gamma | | | | |
| −2.0833333 | PNMA2 | paraneoplastic Ma antigen 2 | 10687 | NM_007257 | Hs.591838 | ENSG00000240694 |
| −2.0408163 | PARK2 | parkin RBR E3 ubiquitin | 5071 | NM_004562 | Hs.132954 | ENSG00000185345 |
| | | protein ligase | | | | |
| −2.1276596 | PDDC1 | Parkinson disease 7 domain | 347862 | NM_182612 | Hs.218362 | ENSG00000177225 |
| | | containing 1 | | | | |
| 1.3300000 | PARK7 | parkinson protein 7 | 11315 | NM_001123377 | Hs.419640 | ENSG00000116288 |
| −2.0408163 | LOC100287846 | patched 1 pseudogene | 100287846 | NR_037168 | Hs.21550 | |
| −2.7027027 | PTCHD4 | patched domain containing 4 | 442213 | NM_001013732 | Hs.659409 | ENSG00000244694 |
| 4.0900000 | PDZK1IP1 | PDZK1 interacting protein 1 | 10158 | NM_005764 | Hs.431099 | ENSG00000162366 |
| 1.6200000 | PEPD | peptidase D | 5184 | NM_000285 | Hs.36473 | ENSG00000124299 |
| −2.1739130 | PPIL6 | peptidylprolyl isomerase | 285755 | NM_001111298 | Hs.32234 | ENSG00000185250 |
| | | (cyclophilin)-like 6 | | | | |
| −2.0833333 | PPIEL | peptidylprolyl isomerase E- | 728448 | NR_003929 | Hs.472508 | ENSG00000182109 |
| | | like pseudogene | | | | |
| −1.4925373 | PER2 | period circadian clock 2 | 8864 | NM_003894 | Hs.58756 | ENSG00000132326 |
| 1.8600000 | PRDX3 | peroxiredoxin 3 | 10935 | NM_001302272 | Hs.523302 | ENSG00000165672 |
| 1.8600000 | PRDX4 | peroxiredoxin 4 | 10549 | NM_006406 | Hs.83383 | ENSG00000123131 |
| −1.8867925 | PXMP4 | peroxisomal membrane | 11264 | NM_007238 | Hs.654857 | ENSG00000101417 |
| | | protein 4, 24 kDa | | | | |
| −1.3698630 | PHF12 | PHD finger protein 12 | 57649 | NM_001033561 | Hs.444173 | ENSG00000109118 |
| −1.7543860 | PHACTR4 | phosphatase and actin | 65979 | NM_001048183 | Hs.225641 | ENSG00000204138 |
| | | regulator 4 | | | | |
| 1.3900000 | PIGY | phosphatidylinositol glycan | 84992 | NM_001042616 | Hs.26136 | ENSG00000255072 |
| | | anchor biosynthesis, class Y | | | | |
| −1.7857143 | PLCXD1 | phosphatidylinositol-specific | 55344 | NM_018390 | Hs.522568 | ENSG00000182378 |
| | | phospholipase C, X domain | | | | |
| | | containing 1 | | | | |
| 1.6000000 | PISD | phosphatidylserine | 23761 | NM_014338 | Hs.420559 | ENSG00000241878 |
| | | decarboxylase | | | | |
| −2.1276596 | PDE4C | phosphodiesterase 4C, | 5143 | NM_000923 | Hs.132584 | ENSG00000105650 |
| | | CAMP-specific | | | | |
| −2.3809524 | PDE6A | phosphodiesterase 6A, | 5145 | NM_000440 | Hs.567314 | ENSG00000132915 |
| | | CGMP-specific, rod, alpha | | | | |
| −3.4482759 | PGM5P2 | phosphoglucomutase 5 | 595135 | NR_002836 | Hs.571593 | ENSG00000277778 |
| | | pseudogene 2 | | | | |
| −2.0833333 | PLA2G4E | phospholipase A2, group IVE | 123745 | NM_001080490 | Hs.668060 | ENSG00000188089 |
| −1.7241379 | PHAX | phosphorylated adaptor for | 51808 | NM_032177 | Hs.555731 | ENSG00000164902 |
| | | RNA export | | | | |
| 1.3900000 | PYURF | PIGY upstream reading frame | 100996939 | NM_032906 | Hs.26136 | ENSG00000145337 |
| −2.0408163 | PNN | pinin, desmosome associated | 5411 | NM_002687 | Hs.409965 | ENSG00000100941 |
| | | protein | | | | |
| −2.2222222 | PLEKHA5 | pleckstrin homology domain | 54477 | NM_001143821 | Hs.188614 | ENSG00000052126 |
| | | containing, family A member 5 | | | | |
| −1.9230769 | PLEKHG2 | pleckstrin homology domain | 64857 | NM_022835 | Hs.631574 | ENSG00000090924 |
| | | containing, family G (with | | | | |
| | | RhoGef domain) member 2 | | | | |
| −2.5000000 | PABPC1P2 | poly(A) binding protein, | 728773 | NR_026904 | Hs.334462 | ENSG00000198526 |
| | | cytoplasmic 1 pseudogene 2 | | | | |
| −1.4492754 | PCGF3 | polycomb group ring finger 3 | 10336 | NM_006315 | Hs.144309 | ENSG00000185619 |
| −1.5625000 | POLE3 | polymerase (DNA directed), | 54107 | NM_001278255 | Hs.108112 | ENSG00000148229 |
| | | epsilon 3, accessory subunit | | | | |
| −1.7543860 | POLH | polymerase (DNA directed), eta | 5429 | NM_001291969 | Hs.655467 | ENSG00000170734 |
| 1.4800000 | POLD4 | polymerase (DNA-directed), | 57804 | NM_001256870 | Hs.523829 | ENSG00000175482 |
| | | delta 4, accessory subunit | | | | |
| 1.3500000 | POLR2D | polymerase (RNA) II (DNA | 5433 | NM_004805 | Hs.715348 | ENSG00000144231 |
| | | directed) polypeptide D | | | | |
| 1.4700000 | PTBP1 | polypyrimidine tract binding | 5725 | NM_002819 | Hs.172550 | ENSG00000011304 |
| | | protein 1 | | | | |
| −1.8518519 | PBDC1 | polysaccharide biosynthesis | 51260 | NM_001300888 | Hs.370100 | ENSG00000102390 |
| | | domain containing 1 | | | | |
| −1.5384615 | KCMF1 | potassium channel | 56888 | NM_020122 | Hs.654968 | ENSG00000176407 |
| | | modulatory factor 1 | | | | |
| −2.3809524 | KCNJ5 | potassium channel, inwardly | 3762 | NM_000890 | Hs.444595 | ENSG00000120457 |
| | | rectifying subfamily J, | | | | |
| | | member 5 | | | | |
| −2.2222222 | KCNA7 | potassium channel, voltage | 3743 | NM_031886 | Hs.306973 | ENSG00000104848 |
| | | gated shaker related | | | | |
| | | subfamily A, member 7 | | | | |
| 2.2727273 | POU2AF1 | POU class 2 associating | 5450 | NM_006235 | Hs.654525 | ENSG00000110777 |
| | | factor 1 | | | | |
| −2.5000000 | POU5F1 | POU class 5 homeobox 1 | 5460 | NM_001173531 | Hs.249184 | ENSG00000204531 |
| 1.6600000 | PQLC3 | PQ loop repeat containing 3 | 130814 | NM_001282710 | Hs.274415 | ENSG00000162976 |
| −1.8518519 | PRDM7 | PR domain containing 7 | 11105 | NM_001098173 | Hs.406695 | ENSG00000126856 |
| −1.7857143 | PRPF3 | pre-mRNA processing factor 3 | 9129 | NM_004698 | Hs.11776 | ENSG00000117360 |
| −2.0408163 | PRPF38B | pre-mRNA processing factor 38B | 55119 | NM_018061 | Hs.342307 | ENSG00000134186 |
| 1.3100000 | PRPF8 | pre-mRNA processing factor 8 | 10594 | NM_006445 | Hs.181368 | ENSG00000174231 |
| −2.4390244 | PRICKLE2-AS3 | PRICKLE2 antisense RNA 3 | 100874243 | NR_046702 | Hs.670840 | ENSG00000226017 |
| −2.2222222 | PRKAR2A-AS1 | PRKAR2A antisense RNA 1 | 100506637 | NR_109996 | Hs.634259 | ENSG00000224424 |
| −2.0408163 | PRKX-AS1 | PRKX antisense RNA 1 | 100873944 | NR_046643 | | ENSG00000236188 |
| 1.5600000 | POP7 | processing of precursor 7, | 10248 | NM_005837 | Hs.416994 | ENSG00000172336 |
| | | ribonuclease P/MRP subunit | | | | |
| | | (S. cerevisiae) | | | | |
| −1.9230769 | PFN1P2 | profilin 1 pseudogene 2 | 767846 | NR_003242 | Hs.657186 | ENSG00000270392 |
| 1.3700000 | PDCD6IP | programmed cell death 6 | 10015 | NM_001162429 | Hs.475896 | ENSG00000170248 |
| | | interacting protein | | | | |
| 1.3600000 | PHB2 | prohibitin 2 | 11331 | NM_001144831 | Hs.504620 | ENSG00000215021 |
| −2.0000000 | PRR11 | proline rich 11 | 55771 | NM_018304 | Hs.631750 | ENSG00000068489 |
| −1.5384615 | PRRC2C | proline-rich coiled-coil 2C | 23215 | NM_015172 | Hs.494614 | ENSG00000117523 |
| −2.8571429 | PART1 | prostate androgen-regulated | 25859 | NM_001039499 | Hs.146312 | ENSG00000152931 |
| | | transcript 1 (non-protein | | | | |
| | | coding) | | | | |
| −2.3809524 | PRNCR1 | prostate cancer associated | 101867536 | NR_109833 | Hs.652970 | ENSG00000224722 |
| | | non-coding RNA 1 | | | | |
| −1.8181818 | PCAT19 | prostate cancer associated | 100505495 | NR_040109 | Hs.648878 | ENSG00000267107 |
| | | transcript 19 (non-protein | | | | |
| | | coding) | | | | |
| 1.5700000 | PSMD14 | proteasome (prosome, | 10213 | NM_005805 | Hs.740477 | ENSG00000115233 |
| | | macropain) 26S subunit, non- | | | | |
| | | ATPase, 14 | | | | |
| 1.4300000 | PSMD9 | proteasome (prosome, | 5715 | NM_001261400 | Hs.131151 | ENSG00000110801 |
| | | macropain) 26S subunit, non- | | | | |
| | | ATPase, 9 | | | | |
| 1.7900000 | PSMB5 | proteasome (prosome, | 5693 | NM_001130725 | Hs.422990 | ENSG00000100804 |
| | | macropain) subunit, beta | | | | |
| | | type, 5 | | | | |
| −2.1276596 | PIN4P1 | protein (peptidylprolyl | 728758 | NR_003571 | Hs.658099 | ENSG00000227973 |
| | | cis/trans isomerase) NIMA- | | | | |
| | | interacting, 4 pseudogene 1 | | | | |
| −2.0000000 | PRKXP1 | protein kinase, X-linked, | 441733 | NR_073405 | Hs.12250 | ENSG00000270127 |
| | | pseudogene 1 | | | | |
| 1.5900000 | PPP1R37 | protein phosphatase 1, | 284352 | NM_019121 | Hs.285363 | ENSG00000104866 |
| | | regulatory subunit 37 | | | | |
| 1.8700000 | PPP2CB | protein phosphatase 2, | 5516 | NM_001009552 | Hs.491440 | ENSG00000104695 |
| | | catalytic subunit, beta | | | | |
| | | isozyme | | | | |
| 1.2900000 | PPP3CB | protein phosphatase 3, | 5532 | NM_001142353 | Hs.500067 | ENSG00000107758 |
| | | catalytic subunit, beta | | | | |
| | | isozyme | | | | |
| −1.7857143 | PPM1K | protein phosphatase, | 152926 | NM_152542 | Hs.43744 | ENSG00000163644 |
| | | Mg2+/Mn2+ dependent, 1K | | | | |
| −2.0408163 | PTK6 | protein tyrosine kinase 6 | 5753 | NM_001256358 | Hs.51133 | ENSG00000101213 |
| −1.7241379 | PTPN14 | protein tyrosine phosphatase, | 5784 | NM_00540 | Hs.193557 | ENSG00000152104 |
| | | non-receptor type 14 | | | | |
| 1.4100000 | PTPN9 | protein tyrosine phosphatase, | 5780 | NM_002833 | Hs.445775 | ENSG00000169410 |
| | | non-receptor type 9 | | | | |
| −2.2727273 | PCDH11X | protocadherin 11 X-linked | 27328 | NM_001168360 | Hs.655673 | ENSG00000102290 |
| −2.3809524 | PCDH11Y | protocadherin 11 Y-linked | 83259 | NM_001278619 | Hs.661308 | ENSG00000099715 |
| −2.1739130 | PCDHB9 | protocadherin beta 9 | 56127 | NM_019119 | Hs.662726 | ENSG00000177839 |
| −1.8181818 | PCBD2 | pterin-4 alpha-carbinolamine | 84105 | NM_032151 | Hs.710014 | ENSG00000132570 |
| | | dehydratase/dimerization | | | | |
| | | cofactor of hepatocyte | | | | |
| | | nuclear factor 1 alpha (TCF1) 2 | | | | |
| −1.8867925 | PTGES2-AS1 | PTGES2 antisense RNA 1 | 389791 | NM_001013652 | Hs.632678 | ENSG00000232850 |
| | | (head to head) | | | | |
| −1.7241379 | PPFIBP1 | PTPRF interacting protein, | 8496 | NM_001198915 | Hs.172445 | ENSG00000110841 |
| | | binding protein 1 (liprin beta 1) | | | | |
| −1.9607843 | PTPRG-AS1 | PTPRG antisense RNA 1 | 100506994 | NR_038281 | Hs.656620 | ENSG00000241472 |
| −1.9607843 | LOC100506127 | putative uncharacterized | 100506127 | NM_001013634 | Hs.503319 | ENSG00000179240 |
| | | protein FLJ37770-like | | | | |
| 2.0800000 | PYCARD | PYD and CARD domain | 29108 | NM_013258 | Hs.499094 | ENSG00000103490 |
| | | containing | | | | |
| 2.1000000 | PYGO2 | pygopus family PHD finger 2 | 90780 | NM_138300 | Hs.533597 | ENSG00000163348 |
| −1.5873016 | PDXDC2P | pyridoxal-dependent | 283970 | NM_199134 | Hs.513695 | ENSG00000196696 |
| | | decarboxylase domain | | | | |
| | | containing 2, pseudogene | | | | |
| −1.4925373 | PGPEP1 | pyroglutamyl-peptidase I | 54858 | NM_001300927 | Hs.131776 | ENSG00000130517 |
| −2.0000000 | PDP2 | pyruvate dehyrogenase | 57546 | NM_020786 | Hs.632214 | ENSG00000172840 |
| | | phosphatase catalytic subunit 2 | | | | |
| 1.5100000 | QTRT1 | queuine tRNA- | 81890 | NM_031209 | Hs.631638 | ENSG00000213339 |
| | | ribosyltransferase 1 | | | | |
| −1.2820513 | QTRTD1 | queuine tRNA- | 79691 | NM_001256835 | Hs.477162 | ENSG00000151576 |
| | | ribosyltransferase domain | | | | |
| | | containing 1 | | | | |
| 1.5700000 | RABAC1 | Rab acceptor 1 (prenylated) | 10567 | NM_006423 | Hs.11417 | ENSG00000105404 |
| −1.4925373 | RAB11FIP4 | RAB11 family interacting | 84440 | NM_001303542 | Hs.406788 | ENSG00000131242 |
| | | protein 4 (class II) | | | | |
| −1.7857143 | RAB12 | RAB12, member RAS | 201475 | NM_001025300 | Hs.270074 | ENSG00000206418 |
| | | oncogene family | | | | |
| −2.1276596 | RAB42 | RAB42, member RAS | 115273 | NM_001193532 | Hs.652321 | ENSG00000188060 |
| | | oncogene family | | | | |
| 1.4000000 | RAB5C | RAB5C, member RAS | 5878 | NM_001252039 | Hs.650382 | ENSG00000108774 |
| | | oncogene family | | | | |
| −1.4705882 | RADI | RAD1 checkpoint DNA | 5810 | NM_001033673 | Hs.38114 | ENSG00000113456 |
| | | exonuclease | | | | |
| 1.5300000 | RALY | RALY heterogeneous nuclear | 22913 | NM_007367 | Hs.136947 | ENSG00000125970 |
| | | ribonucleoprotein | | | | |
| −2.7027027 | RAMP2-AS1 | RAMP2 antisense RNA 1 | 100190938 | NR_024461 | Hs.655265 | ENSG00000197291 |
| 2.2400000 | RHOG | ras homolog family member G | 391 | NM_001665 | Hs.501728 | ENSG00000177105 |
| −2.7777778 | RASAL2-AS1 | RASAL2 antisense RNA 1 | 100302401 | NR_027982 | Hs.736117 | ENSG00000224687 |
| 1.8500000 | RASL11A | RAS-like, family 11, member A | 387496 | NM_206827 | Hs.192131 | ENSG00000122035 |
| 1.9700000 | RAC2 | ras-related C3 botulinum | 5880 | NM_002872 | Hs.517601 | ENSG00000128340 |
| | | toxin substrate 2 (rho family, | | | | |
| | | small GTP binding protein | | | | |
| | | Rac2) | | | | |
| 1.6400000 | RITA1 | RBPJ interacting and tubulin | 84934 | NM_001286215 | Hs.524762 | ENSG00000139405 |
| | | associated 1 | | | | |
| −1.3888889 | RIPK1 | receptor (TNFRSF)- | 8737 | NM_003804 | Hs.519842 | ENSG00000137275 |
| | | interacting serine-threonine | | | | |
| | | kinase 1 | | | | |
| 2.0100000 | RGS14 | regulator of G-protein | 10636 | NM_006480 | Hs.9347 | ENSG00000169220 |
| | | signaling 14 | | | | |
| 1.4200000 | RGS3 | regulator of G-protein | 5998 | NM_001276260 | Hs.494875 | ENSG00000138835 |
| | | signaling 3 | | | | |
| 1.6700000 | RFX1 | regulatory factor X, 1 | 5989 | NM_002918 | Hs.655215 | ENSG00000132005 |
| | | (influences HLA class II | | | | |
| | | expression) | | | | |
| 1.4800000 | RTN3 | reticulon 3 | 10313 | NM_001265589 | Hs.743229 | ENSG00000133318 |
| −1.8181818 | RFPL1S | RFPL1 antisense RNA 1 | 10740 | NR_002727 | Hs.167750 | ENSG00000225465 |
| −1.7241379 | RHD | Rh blood group, D antigen | 6007 | NM_001127691 | Hs.449968 | ENSG00000187010 |
| −2.0000000 | RHBG | Rh family, B glycoprotein | 57127 | NM_001256395 | Hs.131835 | ENSG00000132677 |
| | | (gene/pseudogene) | | | | |
| −1.2987013 | ARHGAP26 | Rho GTPase activating | 23092 | NM_001135608 | Hs.654668 | ENSG00000145819 |
| | | protein 26 | | | | |
| −2.0833333 | RHBDL2 | rhomboid, veinlet-like 2 | 54933 | NM_001304746 | Hs.524626 | ENSG00000158315 |
| | | (Drosophila) | | | | |
| 2.0000000 | RAVER1 | ribonucleoprotein, PTB- | 125950 | NM_133452 | Hs.744952 | ENSG00000161847 |
| | | binding 1 | | | | |
| 1.6500000 | RPL18A | ribosomal protein L18a | 6142 | NM_000980 | Hs.337766 | ENSG00000105640 |
| 1.6300000 | RPL28 | ribosomal protein L28 | 6158 | NM_000991 | Hs.652114 | ENSG00000108107 |
| 1.5400000 | RPL41 | ribosomal protein L41 | 6171 | NM_001035267 | Hs.112553 | ENSG00000229117 |
| 1.5900000 | RPS12 | ribosomal protein S12 | 6206 | NM_00106 | Hs.546289 | ENSG00000112306 |
| −1.7241379 | RPS15AP10 | ribosomal protein S15a | 728963 | NR_026768 | Hs.675157 | ENSG00000225447 |
| | | pseudogene 10 | | | | |
| 1.9500000 | RPS6KA4 | ribosomal protein S6 kinase, | 8986 | NM_001006944 | Hs.105584 | ENSG00000162302 |
| | | 90 kDa, polypeptide 4 | | | | |
| 1.8700000 | RPLP1 | ribosomal protein, large, P1 | 6176 | NM_001003 | Hs.356502 | ENSG00000137818 |
| −1.6129032 | RRP15 | ribosomal RNA processing | 51018 | NM_016052 | Hs.660109 | ENSG00000067533 |
| | | 15 homolog (S. cerevisiae) | | | | |
| 1.9700000 | RNF126 | ring finger protein 126 | 55658 | NM_017876 | Hs.69554 | ENSG00000070423 |
| 1.5400000 | RNF167 | ring finger protein 167 | 26001 | NM_015528 | Hs.7158 | ENSG00000108523 |
| −2.1276596 | RNF207 | ring finger protein 207 | 388591 | NM_173795 | Hs.716549 | ENSG00000158286 |
| −1.2195122 | RNF216 | ring finger protein 216 | 54476 | NM_019011 | Hs.487458 | ENSG00000011275 |
| −2.5641026 | RNF222 | ring finger protein 222 | 643904 | NM_001146684 | Hs.526550 | ENSG00000189051 |
| −1.9607843 | RBM25 | RNA binding motif protein 25 | 58517 | NM_021239 | Hs.531106 | ENSG00000119707 |
| −2.1276596 | RBM34 | RNA binding motif protein 34 | 23029 | NM_001161533 | Hs.535224 | ENSG00000188739 |
| 1.4100000 | RBM42 | RNA binding motif protein 42 | 79171 | NM_024321 | Hs.5086 | ENSG00000126254 |
| −1.6666667 | RBM43 | RNA binding motif protein 43 | 375287 | NM_198557 | Hs.302442 | ENSG00000184898 |
| −1.8518519 | RBM48 | RNA binding motif protein 48 | 84060 | NM_032120 | Hs.21590 | ENSG00000127993 |
| −1.6129032 | RBMS2 | RNA binding motif, single | 5939 | NM_002898 | Hs.505729 | ENSG00000076067 |
| | | stranded interacting protein 2 | | | | |
| −2.3255814 | RNF144A-AS1 | RNF144A antisense RNA 1 | 386597 | NR_033997 | Hs.559010 | ENSG00000228203 |
| −3.0303030 | ROR1-AS1 | ROR1 antisense RNA 1 | 101927034 | NR_110665 | Hs.680824 | ENSG00000223949 |
| −1.4492754 | SAP30BP | SAP30 binding protein | 29115 | NM_001301839 | Hs.655088 | ENSG00000161526 |
| −1.8867925 | SFMBT2 | Scm-like with four mbt | 57713 | NM_001018039 | Hs.407983 | ENSG00000198879 |
| | | domains 2 | | | | |
| −1.5151515 | SDE2 | SDE2 telomere maintenance | 163859 | NM_152608 | Hs.520192 | ENSG00000143751 |
| | | homolog (S. pombe) | | | | |
| 1.6000000 | SEMA4A | sema domain, | 64218 | NM_001193300 | Hs.408846 | ENSG00000196189 |
| | | immunoglobulin domain (Ig), | | | | |
| | | transmembrane domain (TM) | | | | |
| | | and short cytoplasmic | | | | |
| | | domain, (semaphorin) 4A | | | | |
| 1.7900000 | SEMA4B | sema domain, | 10509 | NM_020210 | Hs.474935 | ENSG00000185033 |
| | | immunoglobulin domain (Ig), | | | | |
| | | transmembrane domain (TM) | | | | |
| | | and short cytoplasmic | | | | |
| | | domain, (semaphorin) 4B | | | | |
| −1.6393443 | SEPT7P2 | septin 7 pseudogene 2 | 641977 | NR_024271 | Hs.723477 | ENSG00000214765 |
| 1.5500000 | SQSTM1 | sequestosome 1 | 8878 | NM_001142298 | Hs.724025 | ENSG00000161011 |
| 1.6700000 | SHMT2 | serine | 6472 | NM_001166356 | Hs.741179 | ENSG00000182199 |
| | | hydroxymethyltransferase 2 | | | | |
| | | (mitochondrial) | | | | |
| 2.0700000 | SPINT1 | serine peptidase inhibitor, | 6692 | NM_001032367 | Hs.233950 | ENSG00000166145 |
| | | Kunitz type 1 | | | | |
| −1.4925373 | SRSF10 | serine/arginine-rich splicing | 10772 | NM_001191005 | Hs.3530 | ENSG00000188529 |
| | | factor 10 | | | | |
| −1.8181818 | SRSF11 | serine/arginine-rich splicing | 9295 | NM_001190987 | Hs.479693 | ENSG00000116754 |
| | | factor 11 | | | | |
| −1.8181818 | SRSF4 | serine/arginine-rich splicing | 6429 | NM_005626 | Hs.469970 | ENSG00000116350 |
| | | factor 4 | | | | |
| −2.2727273 | SAA2 | serum amyloid A2 | 6289 | NM_001127380 | Hs.731376 | ENSG00000134339 |
| −1.7241379 | SMYD4 | SET and MYND domain | 114826 | NM_052928 | Hs.514602 | ENSG00000186532 |
| | | containing 4 | | | | |
| −1.4492754 | SETMAR | SET domain and mariner | 6419 | NM_001243723 | Hs.475300 | ENSG00000170364 |
| | | transposase fusion gene | | | | |
| −1.8867925 | SCML4 | sex comb on midleg-like 4 | 256380 | NM_001286408 | Hs.486109 | ENSG00000146285 |
| | | (Drosophila) | | | | |
| −2.1739130 | STAC2 | SH3 and cysteine rich domain 2 | 342667 | NM_198993 | Hs.145068 | ENSG00000141750 |
| −1.4925373 | SH3BP5-AS1 | SH3BP5 antisense RNA 1 | 100505696 | NR_046084 | Hs.745026 | ENSG00000224660 |
| −2.0408163 | SHANK2-AS3 | SHANK2 antisense RNA 3 | 220070 | NM_145308 | Hs.326766 | ENSG00000171671 |
| −2.0408163 | LOC100420587 | SHC SH2-domain binding | 100420587 | NR_110759 | Hs.569956 | ENSG00000267243 |
| | | protein 1 pseudogene | | | | |
| −3.0303030 | SHISA9 | shisa family member 9 | 729993 | NM_001145204 | Hs.130661 | ENSG00000237515 |
| −2.2222222 | SGOL1 | shugoshin-like 1 (S. pombe) | 151648 | NM_001012409 | Hs.105153 | ENSG00000129810 |
| 1.9100000 | SIGLEC9 | sialic acid binding Ig-like | 27180 | NM_001198558 | Hs.245828 | ENSG00000129450 |
| | | lectin 9 | | | | |
| 1.6700000 | SPCS1 | signal peptidase complex | 28972 | NM_014041 | Hs.11125 | ENSG00000114902 |
| | | subunit 1 homolog (S. | | | | |
| | | cerevisiae) | | | | |
| 1.5600000 | SRP9 | signal recognition particle | 6726 | NM_001130440 | Hs.511425 | ENSG00000143742 |
| | | 9 kDa | | | | |
| 1.7700000 | SSR2 | signal sequence receptor, beta | 6746 | NM_003145 | Hs.74564 | ENSG00000163479 |
| | | (translocon-associated protein | | | | |
| | | beta) | | | | |
| 1.4000000 | STAT5B | signal transducer and | 6777 | NM_012448 | Hs.595276 | ENSG00000173757 |
| | | activator of transcription 5B | | | | |
| 1.9900000 | SMDT1 | single-pass membrane protein | 91689 | NM_033318 | Hs.306083 | ENSG00000183172 |
| | | with aspartate-rich tail 1 | | | | |
| 2.2700000 | SIRT2 | sirtuin 2 | 22933 | NM_001193286 | Hs.466693 | ENSG00000068903 |
| −1.5384615 | SIRT3 | sirtuin 3 | 23410 | NM_001017524 | Hs.716456 | ENSG00000142082 |
| −2.2222222 | SIX4 | SIX homeobox 4 | 51804 | NM_017420 | Hs.97849 | ENSG00000100625 |
| 1.4600000 | SSNA1 | Sjogren syndrome nuclear | 8636 | NM_003731 | Hs.530314 | ENSG00000176101 |
| | | autoantigen 1 | | | | |
| −2.1739130 | SLFNL1-AS1 | SLFNL1 antisense RNA 1 | 100507178 | NR_037868 | Hs.660056 | |
| 2.7900000 | SLMO2-ATP5E | SLMO2-ATP5E readthrough | 100533975 | NR_037929 | Hs.656865 | ENSG00000236105 |
| 2.9700000 | SLX1A | SLX1 structure-specific | 548593 | NM_001014999 | Hs.729791 | ENSG00000132207 |
| | | endonuclease subunit | | | | |
| | | homolog A (S. cerevisiae) | | | | |
| 2.9700000 | SLX1B | SLX1 structure-specific | 79008 | NM_024044 | Hs.728161 | ENSG00000181625 |
| | | endonuclease subunit | | | | |
| | | homolog B (S. cerevisiae) | | | | |
| −2.0000000 | SNIP1 | Smad nuclear interacting | 79753 | NM_024700 | Hs.47232 | ENSG00000163877 |
| | | protein 1 | | | | |
| 3.2400000 | SCARNA20 | small Cajal body-specific | 677681 | NR_002999 | | ENSG00000252577 |
| | | RNA 20 | | | | |
| −2.1739130 | SGSM1 | small G protein signaling | 129049 | NM_001039948 | Hs.474397 | ENSG00000167037 |
| | | modulator 1 | | | | |
| −2.8571429 | SNHG4 | small nucleolar RNA host | 724102 | NR_003141 | Hs.268939 | ENSG00000015479 |
| | | gene 4 | | | | |
| 3.4900000 | SNORD16 | small nucleolar RNA, C/D | 595097 | NR_002440 | Hs.739034 | ENSG00000174444 |
| | | box 16 | | | | |
| 4.9900000 | SNORD56 | small nucleolar RNA, C/D | 26793 | NR_002739 | | ENSG00000229686 |
| | | box 56 | | | | |
| −2.3809524 | SMG1P7 | SMG1 pseudogene 7 | 100506060 | NR_033959 | Hs.655258 | ENSG00000261556 |
| −1.2987013 | SMU1 | smu-1 suppressor of mec-8 | 55234 | NM_018225 | Hs.655351 | ENSG00000122692 |
| | | and unc-52 homolog (C. | | | | |
| | | elegans) | | | | |
| 2.4700000 | SNAI3 | snail family zinc finger 3 | 333929 | NM_178310 | Hs.673548 | ENSG00000185669 |
| 2.2800000 | SCN1B | sodium channel, voltage | 6324 | NM_001037 | Hs.436646 | ENSG00000105711 |
| | | gated, type I beta subunit | | | | |
| −2.2222222 | SLC14A2 | solute carrier family 14 (urea | 8170 | NM_001242692 | Hs.710927 | ENSG00000132874 |
| | | transporter), member 2 | | | | |
| −1.9607843 | SLC15A1 | solute carrier family 15 | 6564 | NM_005073 | Hs.436893 | ENSG00000088386 |
| | | (oligopeptide transporter), | | | | |
| | | member 1 | | | | |
| 1.8100000 | SLC16A5 | solute carrier family 16 | 9121 | NM_001271765 | Hs.592095 | ENSG00000170190 |
| | | (monocarboxylate | | | | |
| | | transporter), member 5 | | | | |
| 1.8800000 | SLC19A1 | solute carrier family 19 | 6573 | NM_001205206 | Hs.84190 | ENSG00000173638 |
| | | (folate transporter), member 1 | | | | |
| 1.3400000 | SLC25A5 | solute carrier family 25 | 292 | NM_001152 | Hs.632282 | ENSG00000005022 |
| | | (mitochondrial carrier; | | | | |
| | | adenine nucleotide | | | | |
| | | translocator), member 5 | | | | |
| 2.1000000 | SLC25A1 | solute carrier family 25 | 6576 | NM_001256534 | Hs.111024 | ENSG00000100075 |
| | | (mitochondrial carrier; citrate | | | | |
| | | transporter), member 1 | | | | |
| −2.3809524 | SLC25A5 | solute carrier family 25 | 10166 | NM_014252 | Hs.646645 | ENSG00000102743 |
| | | (mitochondrial carrier; | | | | |
| | | ornithine transporter) member 15 | | | | |
| 1.8400000 | SLC25A11 | solute carrier family 25 | 8402 | NM_001165417 | Hs.184877 | ENSG00000108528 |
| | | (mitochondrial carrier; | | | | |
| | | oxoglutarate carrier), member 11 | | | | |
| 1.6100000 | SLC25A3 | solute carrier family 25 | 5250 | NM_002635 | Hs.290404 | ENSG00000075415 |
| | | (mitochondrial carrier; | | | | |
| | | phosphate carrier), member 3 | | | | |
| −1.5873016 | SLC25A32 | solute carrier family 25 | 81034 | NM_030780 | Hs.532265 | ENSG00000164933 |
| | | (mitochondrial folate carrier), | | | | |
| | | member 32 | | | | |
| 1.6500000 | SLC25A38 | solute carrier family 25, | 54977 | NM_017875 | Hs.369615 | ENSG00000144659 |
| | | member 38 | | | | |
| 1.4800000 | SLC27A1 | solute carrier family 27 (fatty | 376497 | NM_198580 | Hs.363138 | ENSG00000130304 |
| | | acid transporter), member 1 | | | | |
| −2.8571429 | SLC28A2 | solute carrier family 28 | 9153 | NM_004212 | Hs.367833 | ENSG00000137860 |
| | | (concentrative nucleoside | | | | |
| | | transporter), member 2 | | | | |
| −1.7857143 | SLC31A1 | solute carrier family 31 | 1317 | NM_001859 | Hs.532315 | ENSG00000136868 |
| | | (copper transporter), member 1 | | | | |
| 1.6600000 | SLC35A1 | solute carrier family 35 | 10559 | NM_001168398 | Hs.423163 | ENSG00000164414 |
| | | (CMP-sialic acid transporter), | | | | |
| | | member A1 | | | | |
| 1.6700000 | SLC35A5 | solute carrier family 35, | 55032 | NM_017945 | Hs.237480 | ENSG00000138459 |
| | | member A5 | | | | |
| −2.1739130 | SLC36A2 | solute carrier family 36 | 153201 | NM_181776 | Hs.483877 | ENSG00000186335 |
| | | (proton/amino acid | | | | |
| | | symporter), member 2 | | | | |
| −2.0000000 | SLC38A1 | solute carrier family 38, | 81539 | NM_001077484 | Hs.533770 | ENSG00000111371 |
| | | member 1 | | | | |
| 1.5500000 | SLC39A3 | solute carrier family 39 (zinc | 29985 | NM_144564 | Hs.515046 | ENSG00000141873 |
| | | transporter), member 3 | | | | |
| −1.7857143 | SLC4A8 | solute carrier family 4, | 9498 | NM_001039960 | Hs.4749 | ENSG00000050438 |
| | | sodium bicarbonate | | | | |
| | | cotransporter, member 8 | | | | |
| 2.1500000 | SLC40A1 | solute carrier family 40 (iron- | 30061 | NM_014585 | Hs.643005 | ENSG00000138449 |
| | | regulated transporter), | | | | |
| | | member 1 | | | | |
| −1.7857143 | SLC41A2 | solute carrier family 41 | 84102 | NM_032148 | Hs.577463 | ENSG00000136052 |
| | | (magnesium transporter), | | | | |
| | | member 2 | | | | |
| 1.7100000 | SLC41A3 | solute carrier family 41, | 54946 | NM_001008485 | Hs.573007 | ENSG00000114544 |
| | | member 3 | | | | |
| −2.2727273 | SLC44A4 | solute carrier family 44, | 80736 | NM_001178044 | Hs.335355 | ENSG00000204385 |
| | | member 4 | | | | |
| −2.3809524 | SLC5A5 | solute carrier family 5 | 6528 | NM_000453 | Hs.584804 | ENSG00000105641 |
| | | (sodium/iodide | | | | |
| | | cotransporter), member 5 | | | | |
| −2.0408163 | SLC7A5P2 | solute carrier family 7 (amino | 387254 | NR_002594 | Hs.448808 | |
| | | acid transporter light chain, L | | | | |
| | | system), member 5 | | | | |
| | | pseudogene 2 | | | | |
| −2.1276596 | LOC284379 | solute carrier family 7 | 284379 | NR_002938 | Hs.631571 | ENSG00000268864 |
| | | (cationic amino acid | | | | |
| | | transporter, y+ system), | | | | |
| | | member 3 pseudogene | | | | |
| −1.7241379 | SLC7A14 | solute carrier family 7, | 57709 | NM_020949 | Hs.596660 | ENSG00000013293 |
| | | member 14 | | | | |
| −2.6315789 | SLC9A4 | solute carrier family 9, | 389015 | NM_001011552 | Hs.447686 | ENSG00000180251 |
| | | subfamily A (NHE4, cation | | | | |
| | | proton antiporter 4), member 4 | | | | |
| −1.4925373 | SLC9A7 | solute carrier family 9, | 84679 | NM_001257291 | Hs.91389 | ENSG00000065923 |
| | | subfamily A (NHE7, cation | | | | |
| | | proton antiporter 7), member 7 | | | | |
| 1.9100000 | SORBS3 | sorbin and SH3 domain | 10174 | NM_001018003 | Hs.528572 | ENSG00000120896 |
| | | containing 3 | | | | |
| 1.4800000 | SNX12 | sorting nexin 12 | 29934 | NM_001256185 | Hs.260750 | ENSG00000147164 |
| 1.6700000 | SNX17 | sorting nexin 17 | 9784 | NM_001267059 | Hs.278569 | ENSG00000115234 |
| −2.3255814 | SOX9-AS1 | SOX9 antisense RNA 1 | 400618 | NR_103737 | Hs.657374 | ENSG00000234899 |
| 1.5900000 | SPG21 | spastic paraplegia 21 | 51324 | NM_001127889 | Hs.242458 | ENSG00000090487 |
| | | (autosomal recessive, Mast | | | | |
| | | syndrome) | | | | |
| −1.3888889 | SPAST | spastin | 6683 | NM_014946 | Hs.468091 | ENSG00000021574 |
| −3.2258065 | SPC25 | SPC25, NDC80 kinetochore | 57405 | NM_020675 | Hs.421956 | ENSG00000152253 |
| | | complex component | | | | |
| −1.6666667 | SPDYE5 | speedy/RINGO cell cycle | 442590 | NM_001099435 | Hs.632298 | ENSG00000170092 |
| | | regulator family member E5 | | | | |
| −1.9230769 | SPATS2 | spermatogenesis associated, | 65244 | NM_001293285 | Hs.654826 | ENSG00000123352 |
| | | serine-rich 2 | | | | |
| −2.3809524 | SKA1 | spindle and kinetochore | 220134 | NM_001039535 | Hs.134726 | ENSG00000154839 |
| | | associated complex subunit 1 | | | | |
| 1.5200000 | SF3A2 | splicing factor 3a, subunit 2, | 8175 | NM_007165 | Hs.115232 | ENSG00000104897 |
| | | 66 kDa | | | | |
| −2.0833333 | SREK1 | splicing regulatory | 140890 | NM_001077199 | Hs.49367 | ENSG00000153914 |
| | | glutamine/lysine-rich protein 1 | | | | |
| −1.8867925 | SPRED1 | sprouty-related, EVH1 | 161742 | NM_152594 | Hs.525781 | ENSG00000166068 |
| | | domain containing 1 | | | | |
| −1.9607843 | SRRM2-AS1 | SRRM2 antisense RNA 1 | 100128788 | NR_027274 | Hs.311208 | ENSG00000205913 |
| −2.3255814 | STAU2-AS1 | STAU2 antisense RNA 1 | 100128126 | NR_038406 | Hs.679921 | ENSG00000253302 |
| −1.9230769 | STEAP2 | STEAP family member 2, | 261729 | NM_001040665 | Hs.489051 | ENSG00000157214 |
| | | metalloreductase | | | | |
| −3.1250000 | SCD5 | stearoyl-CoA desaturase 5 | 79966 | NM_001037582 | Hs.379191 | ENSG00000145284 |
| −1.8867925 | STAR | steroidogenic acute regulatory | 6770 | NM_000349 | Hs.521535 | ENSG00000147465 |
| | | protein | | | | |
| −2.0000000 | STRIP2 | striatin interacting protein 2 | 57464 | NM_001134336 | Hs.489988 | ENSG00000128578 |
| 1.9000000 | SDHAF1 | succinate dehydrogenase | 644096 | NM_001042631 | Hs.356460 | ENSG00000205138 |
| | | complex assembly factor 1 | | | | |
| 1.5200000 | SDHB | succinate dehydrogenase | 6390 | NM_003000 | Hs.465924 | ENSG00000117118 |
| | | complex, subunit B, iron | | | | |
| | | sulfur (Ip) | | | | |
| −2.2727273 | SUMO1P3 | SUMO1 pseudogene 3 | 474338 | NR_002190 | Hs.621179 | |
| | | (functional) | | | | |
| 1.5200000 | SUV420H2 | suppressor of variegation 4- | 84787 | NM_03270 | Hs.590982 | ENSG00000133247 |
| | | 20 homolog 2 (Drosophila) | | | | |
| −1.9230769 | SFTPB | surfactant protein B | 6439 | NM_000542 | Hs.512690 | ENSG00000168878 |
| −1.4285714 | SUGP2 | SURP and G patch domain | 10147 | NM_001017392 | Hs.515271 | ENSG00000064607 |
| | | containing 2 | | | | |
| −1.7857143 | SWSAP1 | SWIM-type zinc finger 7 | 126074 | NM_175871 | Hs.631619 | ENSG00000173928 |
| | | associated protein 1 | | | | |
| 1.7300000 | SYTL1 | synaptotagmin-like 1 | 84958 | NM_001193308 | Hs.469175 | ENSG00000142765 |
| −1.4492754 | SS18 | synovial sarcoma | 6760 | NM_001007559 | Hs.129261 | ENSG00000141380 |
| | | translocation, chromosome 18 | | | | |
| −2.0000000 | TAF8 | TAF8 RNA polymerase II, | 129685 | NM_138572 | Hs.520122 | ENSG00000137413 |
| | | TATA box binding protein | | | | |
| | | (TBP)-associated factor, | | | | |
| | | 43 kDa | | | | |
| −1.7543860 | TBC1D24 | TBC1 domain family, | 57465 | NM_001199107 | Hs.353087 | ENSG00000162065 |
| | | member 24 | | | | |
| −2.0833333 | TBCCD1 | TBCC domain containing 1 | 55171 | NM_001134415 | Hs.518469 | ENSG00000113838 |
| 1.8600000 | TGDS | TDP-glucose 4,6-dehydratase | 23483 | NM_001304430 | Hs.12393 | ENSG00000088451 |
| −2.5641026 | TEX101 | testis expressed 101 | 83639 | NM_001130011 | Hs.97978 | ENSG00000131126 |
| −1.3698630 | TTC21B | tetratricopeptide repeat | 79809 | NM_024753 | Hs.310672 | ENSG00000123607 |
| | | domain 21B | | | | |
| 1.7100000 | THAP11 | THAP domain containing 11 | 57215 | NM_020457 | Hs.632200 | ENSG00000168286 |
| −1.8181818 | THEM4 | thioesterase superfamily | 117145 | NM_053055 | Hs.164070 | ENSG00000159445 |
| | | member 4 | | | | |
| 1.6900000 | TREX1 | three prime repair | 11277 | NM_007248 | Hs.707026 | ENSG00000213689 |
| | | exonuclease 1 | | | | |
| 1.3800000 | TBXAS1 | thromboxane A synthase 1 | 6916 | NM_001061 | Hs.520757 | ENSG00000059377 |
| | | (platelet) | | | | |
| −2.1276596 | TIGD1 | tigger transposable element | 200765 | NM_145702 | Hs.211823 | ENSG00000221944 |
| | | derived 1 | | | | |
| −2.6315789 | TLCD2 | TLC domain containing 2 | 727910 | NM_00116440 | Hs.531005 | ENSG00000185561 |
| −2.1739130 | TLR8-AS1 | TLR8 antisense RNA 1 | 349408 | NR_030727 | Hs.685035 | ENSG00000233338 |
| −1.6949153 | THRIL | TNF and HNRNPL related | 102659353 | NR_110375 | Hs.596464 | |
| | | immunoregulatory long non- | | | | |
| | | coding RNA | | | | |
| −1.6129032 | TRAF3IP1 | TNF receptor-associated | 26146 | NM_001139490 | Hs.631898 | ENSG00000204104 |
| | | factor 3 interacting protein 1 | | | | |
| −2.1739130 | TNFAIP8L2- | TNFAIP8L2-SCNM1 | 100534012 | NM_001204848 | Hs.732060 | ENSG00000163156 |
| | SCNM1 | readthrough | | | | |
| 1.4400000 | TOLLIP | toll interacting protein | 54472 | NM_019009 | Hs.368527 | ENSG00000078902 |
| −1.6949153 | TLR10 | toll-like receptor 10 | 81793 | NM_001017388 | Hs.120551 | ENSG00000174123 |
| 1.3900000 | TOR1A | torsin family 1, member A | 1861 | NM_000113 | Hs.534312 | ENSG00000136827 |
| | | (torsin A) | | | | |
| 2.0000000 | TOR2A | torsin family 2, member A | 27433 | NM_001085347 | Hs.444106 | ENSG00000160404 |
| −1.4925373 | TLK2 | tousled-like kinase 2 | 11011 | NM_001112707 | Hs.445078 | ENSG00000146872 |
| −2.3809524 | TRAF3IP2 | TRAF3 interacting protein 2 | 10758 | NM_001164281 | Hs.561514 | ENSG00000056972 |
| −1.8181818 | TRAF3IP2-AS1 | TRAF3IP2 antisense RNA 1 | 643749 | NR_034108 | Hs.486228 | ENSG00000231889 |
| 1.6800000 | TECR | trans-2,3-enoyl-CoA | 9524 | NM_004868 | Hs.515642 | ENSG00000099797 |
| | | reductase | | | | |
| −1.4492754 | TCERG1 | transcription elongation | 10915 | NM_001040006 | Hs.443465 | ENSG00000113649 |
| | | regulator 1 | | | | |
| −1.8518519 | TVP23A | trans-golgi network vesicle | 780776 | NM_001079512 | Hs.371576 | ENSG00000166676 |
| | | protein 23 homolog A (S. | | | | |
| | | cerevisiae) | | | | |
| −1.9230769 | TVP23C | trans-golgi network vesicle | 201158 | NM_001135036 | Hs.164595 | ENSG00000175106 |
| | | protein 23 homolog C (S. | | | | |
| | | cerevisiae) | | | | |
| −1.8518519 | TRPM6 | transient receptor potential | 140803 | NM_001177310 | Hs.272225 | ENSG00000119121 |
| | | cation channel, subfamily M, | | | | |
| | | member 6 | | | | |
| −2.3255814 | TRPV1 | transient receptor potential | 7442 | NM_018727 | Hs.579217 | ENSG00000196689 |
| | | cation channel, subfamily V, | | | | |
| | | member 1 | | | | |
| −1.4084507 | TIMM10B | translocase of inner | 26515 | NM_0121 | Hs.54943 | ENSG00000132286 |
| | | mitochondrial membrane 10 | | | | |
| | | homolog B (yeast) | | | | |
| 2.2500000 | TSPO | translocator protein (18 kDa) | 706 | NM_000714 | Hs.202 | ENSG00000100300 |
| −2.1276596 | TMIGD2 | transmembrane and | 126259 | NM_001169126 | Hs.263928 | ENSG00000167664 |
| | | immunoglobulin domain | | | | |
| | | containing 2 | | | | |
| 1.3600000 | TMBIM6 | transmembrane BAX | 7009 | NM_001098576 | Hs.743965 | ENSG00000139644 |
| | | inhibitor motif containing 6 | | | | |
| −1.8181818 | TPTE2P1 | transmembrane | 646405 | NM_001126062 | Hs.620592 | |
| | | phosphoinositide 3- | | | | |
| | | phosphatase and tensin | | | | |
| | | homolog 2 pseudogene 1 | | | | |
| 1.8200000 | TMEM120A | transmembrane protein 120A | 83862 | NM_031925 | Hs.488835 | ENSG00000189077 |
| −1.9230769 | TMEM120B | transmembrane protein 120B | 144404 | NM_001080825 | Hs.644504 | ENSG00000188735 |
| 2.3900000 | TMEM141 | transmembrane protein 141 | 85014 | NM_032928 | Hs.356744 | ENSG00000244187 |
| 1.9500000 | TMEM14C | transmembrane protein 14C | 51522 | NM_001165258 | Hs.519557 | ENSG00000111843 |
| 1.7600000 | TMEM150A | transmembrane protein 150A | 129303 | NM_001031738 | Hs.591559 | ENSG00000168890 |
| 2.3300000 | TMEM160 | transmembrane protein 160 | 54958 | NM_017854 | Hs.105606 | ENSG00000130748 |
| 1.5400000 | TMEM165 | transmembrane protein 165 | 55858 | NM_01847: | Hs.479766 | ENSG00000134851 |
| 1.6300000 | TMEM179B | transmembrane protein 179B | 374395 | NM_199337 | Hs.381134 | ENSG00000185475 |
| −1.5625000 | TMEM181 | transmembrane protein 181 | 57583 | NM_020823 | Hs.99145 | ENSG00000146433 |
| −3.0303030 | TMEM212 | transmembrane protein 212 | 389177 | NM_001164436 | Hs.642307 | ENSG00000186329 |
| 1.3600000 | TMEM214 | transmembrane protein 214 | 54867 | NM_001083590 | Hs.533934 | ENSG00000119777 |
| 1.6000000 | TMEM230 | transmembrane protein 230 | 29058 | NM_001009923 | Hs.472024 | ENSG00000089063 |
| 2.5700000 | TMEM256 | transmembrane protein 256 | 254863 | NM_152766 | Hs.730789 | ENSG00000205544 |
| −1.5625000 | TMEM38A | transmembrane protein 38A | 79041 | NM_024074 | Hs.436068 | ENSG00000072954 |
| −1.9607843 | TMEM41B | transmembrane protein 41B | 440026 | NM_001165030 | Hs.594563 | ENSG00000166471 |
| 1.5000000 | TMEM43 | transmembrane protein 43 | 79188 | NM_024334 | Hs.517817 | ENSG00000170876 |
| −1.2987013 | TRIM38 | tripartite motif containing 38 | 10475 | NM_006355 | Hs.584851 | ENSG00000112343 |
| −2.3255814 | TRIM45 | tripartite motif containing 45 | 80263 | NM_001145635 | Hs.301526 | ENSG00000134253 |
| −1.9607843 | TPM3P9 | tropomyosin 3 pseudogene 9 | 147804 | NM_001010856 | Hs.433293 | ENSG00000241015 |
| −2.3255814 | TSIX | TSIX transcript, XIST | 9383 | NR_003255 | Hs.529901 | ENSG00000270641 |
| | | antisense RNA | | | | |
| −1.4084507 | TSPYL1 | TSPY-like 1 | 7259 | NM_003309 | Hs.458358 | ENSG00000189241 |
| 1.7700000 | TTLL12 | tubulin tyrosine ligase-like | 23170 | NM_015140 | Hs.517670 | ENSG00000100304 |
| | | family member 12 | | | | |
| −2.4390244 | TUBA3FP | tubulin, alpha 3f, pseudogene | 113691 | NR_003608 | Hs.585006 | ENSG00000161149 |
| 1.5800000 | TUBB | tubulin, beta class I | 203068 | NM_001293212 | Hs.636480 | ENSG00000196230 |
| −1.7241379 | TUFT1 | tuftelin 1 | 7286 | NM_001126337 | Hs.489922 | ENSG00000143367 |
| −2.3255814 | TP53AIP1 | tumor protein p53 regulated | 63970 | NM_001195194 | Hs.160953 | ENSG00000120471 |
| | | apoptosis inducing protein 1 | | | | |
| 2.0000000 | TPRGIL | tumor protein p63 regulated | 127262 | NM_182752 | Hs.20529 | ENSG00000158109 |
| | | 1-like | | | | |
| −2.0000000 | TSG1 | tumor suppressor TSG1 | 643432 | NR_015362 | Hs.509936 | |
| 1.8000000 | TWF2 | twinfilin actin-binding protein 2 | 11344 | NM_007284 | Hs.436439 | ENSG00000247596 |
| −1.8518519 | TAT | tyrosine aminotransferase | 6898 | NM_000353 | Hs.161640 | ENSG00000198650 |
| 1.8200000 | UQCR10 | ubiquinol-cytochrome c | 29796 | NM_001003684 | Hs.284292 | ENSG00000184076 |
| | | reductase, complex III | | | | |
| | | subunit X | | | | |
| 1.4300000 | UBE3C | ubiquitin protein ligase E3C | 9690 | NM_014671 | Hs.118351 | ENSG00000009335 |
| 1.4500000 | USP21 | ubiquitin specific peptidase 21 | 27005 | NM_001014443 | Hs.8015 | ENSG00000143258 |
| −1.4492754 | USP42 | ubiquitin specific peptidase 42 | 84132 | NM_032172 | Hs.31856 | ENSG00000106346 |
| −1.6393443 | USP49 | ubiquitin specific peptidase 49 | 25862 | NM_001286554 | Hs.593575 | ENSG00000164663 |
| 1.7300000 | UBE2D1 | ubiquitin-conjugating enzyme E2D 1 | 7321 | NM_001204880 | Hs.129683 | ENSG00000072401 |
| −2.2222222 | UBE2Q2P1 | ubiquitin-conjugating enzyme | 388165 | NM_207382 | Hs.498348 | ENSG00000189136 |
| | | E2Q family member 2 | | | | |
| | | pseudogene 1 | | | | |
| 1.4900000 | UBL5 | ubiquitin-like 5 | 59286 | NM_001048241 | Hs.534477 | ENSG00000198258 |
| −2.1739130 | UCKL1-AS1 | UCKL1 antisense RNA 1 | 100113386 | NR_027287 | Hs.551552 | |
| 1.6700000 | B3GALT6 | UDP-Gal:betaGal beta 1,3- | 126792 | NM_080605 | Hs.284284 | ENSG00000176022 |
| | | galactosyltransferase | | | | |
| | | polypeptide 6 | | | | |
| −2.9411765 | B3GNT6 | UDP-GlcNAc:betaGal beta-1,3-N- | 192134 | NM_138706 | Hs.352622 | ENSG00000198488 |
| | | acetylglucosaminyltransferase 6 | | | | |
| −3.7037037 | UGDH-AS1 | UGDH antisense RNA 1 | 100885776 | NR_047679 | Hs.640769 | ENSG00000249348 |
| 1.5300000 | ULK3 | unc-51 like kinase 3 | 25989 | NM_001099436 | Hs.513034 | ENSG00000140474 |
| −2.0833333 | LOC100128233 | uncharacterized | 100128233 | NR_103769 | Hs.497323 | ENSG00000255002 |
| | | LOC100128233 | | | | |
| −2.6315789 | LOC100128288 | uncharacterized | 100128288 | NR_024447 | Hs.549913 | |
| | | LOC100128288 | | | | |
| −2.0408163 | LOC100128398 | uncharacterized | 100128398 | NR_036508 | Hs.655081 | ENSG00000176593 |
| | | LOC100128398 | | | | |
| −2.1276596 | LOC100129940 | uncharacterized | 100129940 | NM_001292023 | Hs.685856 | ENSG00000197301 |
| | | LOC100129940 | | | | |
| −2.6315789 | LOC100130451 | uncharacterized | 100130451 | NM_001242575 | | |
| | | LOC100130451 | | | | |
| −2.1739130 | LOC100131564 | uncharacterized | 100131564 | NR_034089 | Hs.732666 | ENSG00000223745 |
| | | LOC100131564 | | | | |
| −3.0303030 | LOC100131626 | uncharacterized | 100131626 | NR_046369 | Hs.721614 | |
| | | LOC100131626 | | | | |
| −2.4390244 | LOC100132077 | uncharacterized | 100132077 | NR_033937 | Hs.679111 | ENSG00000232063 |
| | | LOC100132077 | | | | |
| −2.1276596 | LOC100190986 | uncharacterized | 100190986 | NR_024456 | Hs.648439 | |
| | | LOC100190986 | | | | |
| −2.3809524 | LOC100287225 | uncharacterized | 100287225 | NR_040074 | Hs.448920 | ENSG00000227115 |
| | | LOC100287225 | | | | |
| −2.0833333 | LOC100379224 | uncharacterized | 100379224 | NR_033341 | Hs.585869 | ENSG00000186019 |
| | | LOC100379224 | | | | |
| 2.2200000 | LOC100505622 | uncharacterized | 100505622 | NR_038332 | Hs.661761 | ENSG00000254887 |
| | | LOC100505622 | | | | |
| −3.2258065 | LOC100505817 | uncharacterized | 100505817 | NR_038340 | Hs.197042 | ENSG00000261780 |
| | | LOC100505817 | | | | |
| −2.0833333 | LOC100506083 | uncharacterized | 100506083 | NR_039997 | Hs.635008 | ENSG00000261777 |
| | | LOC100506083 | | | | |
| −2.2222222 | LOC100506085 | uncharacterized | 100506085 | NR_037878 | Hs.649173 | ENSG00000248319 |
| | | LOC100506085 | | | | |
| −2.0408163 | LOC100506123 | uncharacterized | 100506123 | NR_040097 | Hs.720604 | ENSG00000230606 |
| | | LOC100506123 | | | | |
| −1.7857143 | LOC100506472 | uncharacterized | 100506472 | NR_040535 | Hs.729080 | |
| | | LOC100506472 | | | | |
| −2.0408163 | LOC100506688 | uncharacterized | 100506688 | NM_001242737 | Hs.532063 | ENSG00000215246 |
| | | LOC100506688 | | | | |
| −1.9230769 | LOC100506746 | uncharacterized | 100506746 | NR_038841 | Hs.657766 | ENSG00000163633 |
| | | LOC100506746 | | | | |
| −2.3809524 | LOC100996251 | uncharacterized | 100996251 | NR_103777 | Hs.382067 | ENSG00000238198 |
| | | LOC100996251 | | | | |
| −2.3809524 | LOC100996351 | uncharacterized | 100996351 | NR_110670 | | ENSG00000267551 |
| | | LOC100996351 | | | | |
| −3.0303030 | LOC101926889 | uncharacterized | 101926889 | NR_109994 | Hs.585997 | |
| | | LOC101926889 | | | | |
| −2.2727273 | LOC101926928 | uncharacterized | 101926928 | NR_1101 | Hs.638788 | ENSG00000258551 |
| | | LOC101926928 | | | | |
| −1.7543860 | LOC101926960 | uncharacterized | 101926960 | NR_104635 | Hs.365692 | |
| | | LOC101926960 | | | | |
| −2.0833333 | LOC101927131 | uncharacterized | 101927131 | NR_110907 | Hs.569654 | ENSG00000262999 |
| | | LOC101927131 | | | | |
| −1.9230769 | LOC101927181 | uncharacterized | 101927181 | NR_108066 | Hs.288853 | ENSG00000175873 |
| | | LOC101927181 | | | | |
| −2.0833333 | LOC101927257 | uncharacterized | 101927257 | NR_109965 | Hs.662725 | ENSG00000232564 |
| | | LOC101927257 | | | | |
| −2.3255814 | LOC101927274 | uncharacterized | 101927274 | NR_110751 | Hs.591168 | ENSG00000249383 |
| | | LOC101927274 | | | | |
| −3.4482759 | LOC101927374 | uncharacterized | 101927374 | NR_110133 | Hs.570644 | |
| | | LOC101927374 | | | | |
| −2.3809524 | LOC101927476 | uncharacterized | 101927476 | NR_110386 | Hs.522607 | ENSG00000236393 |
| | | LOC101927476 | | | | |
| −1.3888889 | LOC101927550 | uncharacterized | 101927550 | NR_110102 | Hs.636663 | ENSG00000242687 |
| | | LOC101927550 | | | | |
| −3.2258065 | LOC101927575 | uncharacterized | 101927575 | NR_110995 | Hs.459826 | ENSG00000227463 |
| | | LOC101927575 | | | | |
| −1.7241379 | LOC101927666 | uncharacterized | 101927666 | NR_110809 | Hs.552237 | ENSG00000266290 |
| | | LOC101927666 | | | | |
| −2.2222222 | LOC101927740 | uncharacterized | 101927740 | NR_109890 | Hs.738721 | ENSG00000245812 |
| | | LOC101927740 | | | | |
| −2.5641026 | LOC101927797 | uncharacterized | 101927797 | NR_109925 | Hs.551743 | ENSG00000224141 |
| | | LOC101927797 | | | | |
| −2.3255814 | LOC101927817 | uncharacterized | 101927817 | NR_110931 | Hs.667942 | |
| | | LOC101927817 | | | | |
| −2.7027027 | LOC101927884 | uncharacterized | 101927884 | NR_110281 | Hs.671110 | ENSG00000231172 |
| | | LOC101927884 | | | | |
| −2.2727273 | LOC101928103 | uncharacterized | 101928103 | NR_110292 | Hs.665619 | ENSG00000229267 |
| | | LOC101928103 | | | | |
| −3.2258065 | LOC101928137 | uncharacterized | 101928137 | NR_110130 | Hs.694666 | ENSG00000258123 |
| | | LOC101928137 | | | | |
| −2.4390244 | LOC101928254 | uncharacterized | 101928254 | NR_110182 | Hs.571236 | ENSG00000219445 |
| | | LOC101928254 | | | | |
| −2.4390244 | LOC101928303 | uncharacterized | 101928303 | NR_110698 | Hs.375067 | ENSG00000236155 |
| | | LOC101928303 | | | | |
| −2.2727273 | LOC101928567 | uncharacterized | 101928567 | NR_110839 | Hs.569757 | ENSG00000237057 |
| | | LOC101928567 | | | | |
| −2.0000000 | LOC101928597 | uncharacterized | 101928597 | NR_110091 | Hs.638942. | ENSG00000246394 |
| | | LOC101928597 | | | | |
| −2.3809524 | LOC101928674 | uncharacterized | 101928674 | NR_110845 | Hs.637297 | ENSG00000266970 |
| | | LOC101928674 | | | | |
| −2.9411765 | LOC101928844 | uncharacterized | 101928844 | NR_110740 | Hs.434577 | ENSG00000267709 |
| | | LOC101928844 | | | | |
| −3.0303030 | LOC101928936 | uncharacterized | 101928936 | NR_110867 | Hs.533080 | |
| | | LOC101928936 | | | | |
| −1.8518519 | LOC101929144 | uncharacterized | 101929144 | NR_110745 | Hs.531631 | ENSG00000261615 |
| | | LOC101929144 | | | | |
| −2.5641026 | LOC101929224 | uncharacterized | 101929224 | NR_110787 | Hs.639369 | ENSG00000260088 |
| | | LOC101929224 | | | | |
| −2.3809524 | LOC101929259 | uncharacterized | 101929259 | NR_120424 | Hs.638490 | |
| | | LOC101929259 | | | | |
| −2.5000000 | LOC101929486 | uncharacterized | 101929486 | NR_109868 | Hs.548761 | ENSG00000233048 |
| | | LOC101929486 | | | | |
| −2.7027027 | LOC101929567 | uncharacterized | 101929567 | NR_110257 | Hs.634706 | ENSG00000236008 |
| | | LOC101929567 | | | | |
| −2.3255814 | LOC101929586 | uncharacterized | 101929586 | NR_120363 | Hs.569426 | ENSG00000259175 |
| | | LOC101929586 | | | | |
| −3.0303030 | LOC101929698 | uncharacterized | 101929698 | NR_110619 | Hs.638392 | ENSG00000277301 |
| | | LOC101929698 | | | | |
| −1.9607843 | LOC101929767 | uncharacterized | 101929767 | NR_110868 | Hs.640892 | ENSG00000267002 |
| | | LOC101929767 | | | | |
| −3.0303030 | LOC102467081 | uncharacterized | 102467081 | NR_104662 | | ENSG00000240535 |
| | | LOC102467081 | | | | |
| −2.7027027 | LOC102723769 | uncharacterized | 102723769 | NR_11070 | Hs.652926 | |
| | | LOC102723769 | | | | |
| −2.3255814 | LOC102724927 | uncharacterized | 102724927 | NR_120311 | Hs.364739 | ENSG00000262185 |
| | | LOC102724927 | | | | |
| −2.0000000 | LOC151475 | uncharacterized | 151475 | NR_040038 | Hs.528154 | ENSG00000226125 |
| | | LOC151475 | | | | |
| −2.1276596 | MGC32805 | uncharacterized | 153163 | NR_051996 | Hs.679757 | ENSG00000250328 |
| | | LOC153163 | | | | |
| −2.1739130 | LOC284023 | uncharacterized | 284023 | NR_024349 | Hs.744470 | ENSG00000179859 |
| | | LOC284023 | | | | |
| −2.1276596 | LOC284581 | uncharacterized | 284581 | NR_046097 | | |
| | | LOC284581 | | | | |
| −2.2222222 | LOC284865 | uncharacterized | 284865 | NR_038460 | Hs.638498 | ENSG00000249923 |
| | | LOC284865 | | | | |
| −2.2222222 | LOC284950 | uncharacterized | 284950 | NR_038888 | Hs.570227 | ENSG00000229498 |
| | | LOC284950 | | | | |
| −2.7777778 | LOC286437 | uncharacterized | 286437 | NR_039980 | Hs.656786 | |
| | | LOC286437 | | | | |
| −2.1276596 | LOC339166 | uncharacterized | 339166 | NR_040000 | Hs.736088 | ENSG00000179314 |
| | | LOC339166 | | | | |
| −2.0000000 | LOC339803 | uncharacterized | 339803 | NR_036496 | Hs.252433 | ENSG00000212978 |
| | | LOC339803 | | | | |
| −2.0408163 | LOC389641 | uncharacterized | 389641 | NR_033928 | Hs.591835 | ENSG00000246582 |
| | | LOC389641 | | | | |
| −2.3809524 | FLJ42102 | uncharacterized | 399923 | NM_001001680 | Hs.128191 | ENSG00000172900 |
| | | LOC399923 | | | | |
| −2.2222222 | LOC400958 | uncharacterized | 400958 | NR_036586 | Hs.591565 | ENSG00000237638 |
| | | LOC400958 | | | | |
| −2.3809524 | LOC401052 | uncharacterized | 401052 | NM_001008737 | Hs.662766 | |
| | | LOC401052 | | | | |
| −1.4705882 | LOC401320 | uncharacterized | 401320 | NR_038889 | Hs.561708 | |
| | | LOC401320 | | | | |
| −2.4390244 | FLJ31662 | uncharacterized | 440594 | NR_033966 | Hs.514123 | ENSG00000233907 |
| | | LOC440594 | | | | |
| −2.3809524 | FLJ31104 | uncharacterized | 441072 | NR_102755 | Hs.482141 | ENSG00000227908 |
| | | LOC441072 | | | | |
| −2.9411765 | LOC643406 | uncharacterized | 643406 | NM_175877 | Hs.431161 | ENSG00000230563 |
| | | LOC643406 | | | | |
| −3.0303030 | LOC644919 | uncharacterized | 644919 | NR_109757 | Hs.434414 | |
| | | LOC644919 | | | | |
| −4.3478261 | LOC728752 | uncharacterized | 728752 | NR_036504 | Hs.729762 | ENSG00000267309 |
| | | LOC728752 | | | | |
| −2.0408163 | LOC729732 | uncharacterized | 729732 | NR_047662 | Hs.322761 | |
| | | LOC729732 | | | | |
| −2.1276596 | LOC731424 | uncharacterized | 731424 | NR_037867 | Hs.427740 | |
| | | LOC731424 | | | | |
| −1.4084507 | DKFZP586I1420 | uncharacterized protein | 222161 | NR_002186 | Hs.112423 | ENSG00000235859 |
| | | DKFZp586I1420 | | | | |
| −2.4390244 | FLJ31356 | uncharacterized protein | 403150 | NR_103831 | Hs.562970 | ENSG00000229951 |
| | | FLJ31356 | | | | |
| −1.8518519 | UPK3B | uroplakin 3B | 80761 | NM_030570 | Hs.488861 | ENSG00000243566 |
| −2.2727273 | UTS2B | urotensin 2B | 257313 | NM_198152 | Hs.518492 | ENSG00000188958 |
| 1.6700000 | VPS28 | vacuolar protein sorting 28 | 51160 | NM_016208 | Hs.418175 | ENSG00000160948 |
| | | homolog (S. cerevisiae) | | | | |
| 1.8000000 | VPS51 | vacuolar protein sorting 51 | 738 | NM_013265 | Hs.277517 | ENSG00000149823 |
| | | homolog (S. cerevisiae) | | | | |
| 1.7100000 | VIPR1 | vasoactive intestinal peptide | 7433 | NM_001251882 | Hs.348500 | ENSG00000114812 |
| | | receptor 1 | | | | |
| −1.4705882 | CRK | v-crk avian sarcoma virus | 1398 | NM_005206 | Hs.461896 | ENSG00000167193 |
| | | CT10 oncogene homolog | | | | |
| 1.4500000 | CRKL | v-crk avian sarcoma virus | 1399 | NM_005207 | Hs.5613 | ENSG00000099942 |
| | | CT10 oncogene homolog-like | | | | |
| 1.8000000 | MYCL | v-myc avian | 4610 | NM_001033081 | Hs.437922 | ENSG00000116990 |
| | | myelocytomatosis viral | | | | |
| | | oncogene lung carcinoma | | | | |
| | | derived homolog | | | | |
| −2.5641026 | VNIR2 | vomeronasal 1 receptor 2 | 317701 | NM_173856 | Hs.553684 | ENSG00000196131 |
| 1.7500000 | VBP1 | von Hippel-Lindau binding | 7411 | NM_001303543 | Hs.436803 | ENSG00000155959 |
| | | protein 1 | | | | |
| −2.7777778 | VSIG1 | V-set and immunoglobulin | 340547 | NM_001170553 | Hs.177164 | ENSG00000101842 |
| | | domain containing 1 | | | | |
| −2.5000000 | WFDC8 | WAP four-disulfide core | 90199 | NM_130896 | Hs.116128 | ENSG00000158901 |
| | | domain 8 | | | | |
| −1.5151515 | WHAMM | WAS protein homolog | 123720 | NM_001080435 | Hs.377360 | ENSG00000156232 |
| | | associated with actin, golgi | | | | |
| | | membranes and microtubules | | | | |
| 1.8600000 | WDTC1 | WD and tetratricopeptide | 23038 | NM_001276252 | Hs.469154 | ENSG00000142784 |
| | | repeats 1 | | | | |
| −1.7241379 | WDR55 | WD repeat domain 55 | 54853 | NM_0177 | Hs.286261 | ENSG00000120314 |
| 2.3100000 | WDR83OS | WD repeat domain 83 | 51398 | NM_016145 | Hs.108969 | ENSG00000105583 |
| | | opposite strand | | | | |
| −1.6949153 | WDR92 | WD repeat domain 92 | 116143 | NM_001256476 | Hs.631877 | ENSG00000243667 |
| 1.9000000 | WBP1 | WW domain binding protein 1 | 23559 | NM_012477 | Hs.516114 | ENSG00000239779 |
| −3.4482759 | XKR9 | XK, Kell blood group | 389668 | NM_001011720 | Hs.458938 | ENSG00000221947 |
| | | complex subunit-related | | | | |
| | | family, member 9 | | | | |
| −1.7241379 | XIAP | X-linked inhibitor of | 331 | NM_001167 | Hs.356076 | ENSG00000101966 |
| | | apoptosis, E3 ubiquitin | | | | |
| | | protein ligase | | | | |
| −1.8518519 | XRCC2 | X-ray repair complementing | 7516 | NM_005431 | Hs.647093 | ENSG00000196584 |
| | | defective repair in Chinese | | | | |
| | | hamster cells 2 | | | | |
| −1.9230769 | YES1 | YES proto-oncogene 1, Src | 7525 | NM_005433 | Hs.194148 | ENSG00000176105 |
| | | family tyrosine kinase | | | | |
| 1.3300000 | YIPF1 | Yip1 domain family, member 1 | 54432 | NM_018982 | Hs.11923 | ENSG00000058799 |
| 1.7800000 | YIPF4 | Yip1 domain family, member 4 | 84272 | NM_032312 | Hs.468099 | ENSG00000119820 |
| −1.4492754 | YAF2 | YY1 associated factor 2 | 10138 | NM_001012424 | Hs.649195 | ENSG00000015153 |
| 1.3900000 | YY1 | YY1 transcription factor | 7528 | NM_003403 | Hs.388927 | ENSG00000100811 |
| −1.7241379 | ZFP14 | ZFP14 zinc finger protein | 57677 | NM_001297619 | Hs.35524 | ENSG00000142065 |
| −2.0000000 | ZFP30 | ZFP30 zinc finger protein | 22835 | NM_014898 | Hs.716719 | ENSG00000120784 |
| −2.4390244 | ZFP91-CNTF | ZFP91-CNTF readthrough | 386607 | NM_170768 | Hs.524920 | ENSG00000255073 |
| | | (NMD candidate) | | | | |
| 1.8900000 | ZBTB45 | zinc finger and BTB domain | 84878 | NM_032792 | Hs.515662 | ENSG00000119574 |
| | | containing 45 | | | | |
| −1.8181818 | ZSCAN12 | zinc finger and SCAN | 9753 | NM_001039643 | Hs.134816 | ENSG00000158691 |
| | | domain containing 12 | | | | |
| −1.6949153 | ZSCAN2 | zinc finger and SCAN | 54993 | NM_001007072 | Hs.594023 | ENSG00000176371 |
| | | domain containing 2 | | | | |
| −2.3255814 | ZSCAN22 | zinc finger and SCAN | 342945 | NM_181846 | Hs.388162 | ENSG00000182318 |
| | | domain containing 22 | | | | |
| −2.1739130 | ZC3H12D | zinc finger CCCH-type | 340152 | NM_207360 | Hs.632618 | ENSG00000178199 |
| | | containing 12D | | | | |
| −1.5151515 | ZNF117 | zinc finger protein 117 | 51351 | NM_015852 | Hs.250693 | ENSG00000152926 |
| −1.7857143 | ZNF264 | zinc finger protein 264 | 9422 | NM_003417 | Hs.515634 | ENSG00000083844 |
| −1.7543860 | ZNF286A | zinc finger protein 286A | 57335 | NM_001130842 | Hs.585799 | ENSG00000187607 |
| −1.8181818 | ZNF286B | zinc finger protein 286B | 729288 | NM_001145045 | Hs.534279 | ENSG00000249459 |
| −1.8518519 | ZNF326 | zinc finger protein 326 | 284695 | NM_18178 | Hs.306221 | ENSG00000162664 |
| −2.0833333 | ZNF329 | zinc finger protein 329 | 79673 | NM_024620 | Hs.458377 | ENSG00000181894 |
| −1.5384615 | ZNF417 | zinc finger protein 417 | 147687 | NM_001297734 | Hs.567710 | ENSG00000173480 |
| 1.8500000 | ZNF428 | zinc finger protein 428 | 126299 | NM_182498 | Hs.99093 | ENSG00000131116 |
| −1.4084507 | ZNF44 | zinc finger protein 44 | 51710 | NM_001164276 | Hs.296731 | ENSG00000197857 |
| −2.8571429 | ZNF471 | zinc finger protein 471 | 57573 | NM_020813 | Hs.710590 | ENSG00000196263 |
| −2.5641026 | ZNF483 | zinc finger protein 483 | 158399 | NM_001007169 | Hs.660784 | ENSG00000173258 |
| −2.0000000 | ZNF490 | zinc finger protein 490 | 57474 | NM_020714 | Hs.655860 | ENSG00000188033 |
| −1.9607843 | ZNF527 | zinc finger protein 527 | 84503 | NM_032453 | Hs.590940 | ENSG00000189164 |
| −2.0833333 | ZNF528 | zinc finger protein 528 | 84436 | NM_032423 | Hs.662043 | ENSG00000167555 |
| −1.8867925 | ZNF548 | zinc finger protein 548 | 147694 | NM_001172773 | Hs.126905 | ENSG00000188785 |
| −2.0000000 | ZNF554 | zinc finger protein 554 | 115196 | NM_001102651 | Hs.307043 | ENSG00000172006 |
| −1.7857143 | ZNF562 | zinc finger protein 562 | 54811 | NM_001130031 | Hs.371107 | ENSG00000171466 |
| −2.1276596 | ZNF585B | zinc finger protein 585B | 92285 | NM_152279 | Hs.390568 | ENSG00000245680 |
| −1.4084507 | ZNF587 | zinc finger protein 587 | 84914 | NM_001204817 | Hs.642598 | ENSG00000198466 |
| −1.5151515 | ZNF621 | zinc finger protein 621 | 285268 | NM_001098414 | Hs.19977 | ENSG00000172888 |
| −1.2820513 | ZNF655 | zinc finger protein 655 | 79027 | NM_001009956 | Hs.599798 | ENSG00000197343 |
| −2.7777778 | LOC100131257 | zinc finger protein 655 | 100131257 | NR_034022 | Hs.551110 | |
| | | pseudogene | | | | |
| −1.8867925 | ZNF662 | zinc finger protein 662 | 389114 | NM_001134656 | Hs.720173 | ENSG00000182983 |
| −2.2222222 | ZNF665 | zinc finger protein 665 | 79788 | NM_024733 | Hs.745230 | ENSG00000197497 |
| −2.0833333 | ZNF677 | zinc finger protein 677 | 342926 | NM_182609 | Hs.20506 | ENSG00000197928 |
| −2.0000000 | ZNF713 | zinc finger protein 713 | 349075 | NM_182633 | Hs.660834 | ENSG00000178665 |
| −1.5384615 | ZNF714 | zinc finger protein 714 | 148206 | NM_182515 | Hs.729186 | ENSG00000160352 |
| −1.7857143 | ZNF737 | zinc finger protein 737 | 100129842 | NM_001159293 | Hs.515696 | ENSG00000237440 |
| −2.4390244 | ZNF761 | zinc finger protein 761 | 388561 | NM_001008401 | Hs.433293 | ENSG00000160336 |
| −2.2222222 | ZNF793 | zinc finger protein 793 | 390927 | NM_001013659 | Hs.568010 | ENSG00000188227 |
| −2.0000000 | ZNF814 | zinc finger protein 814 | 730051 | NM_001144989 | Hs.634143 | ENSG00000204514 |
| −1.9607843 | ZNF818P | zinc finger protein 818, | 390963 | NM_001001675 | Hs.444446 | ENSG00000269001 |
| | | pseudogene | | | | |
| −2.0408163 | ZNF850 | zinc finger protein 850 | 342892 | NM_001193552 | Hs.406307 | ENSG00000267041 |
| −2.7777778 | ZKSCAN3 | zinc finger with KRAB and | 80317 | NM_001242894 | Hs.380930 | ENSG00000189298 |
| | | SCAN domains 3 | | | | |
| −1.8518519 | ZCCHC4 | zinc finger, CCHC domain | 29063 | NM_024936 | Hs.278945 | ENSG00000168228 |
| | | containing 4 | | | | |
| 1.5800000 | ZFYVE21 | zinc finger, FYVE domain | 79038 | NM_001198953 | Hs.592322 | ENSG00000100711 |
| | | containing 21 | | | | |
| 1.8100000 | ZNHIT1 | zinc finger, HIT-type | 10467 | NM_006349 | Hs.211079 | ENSG00000106400 |
| | | containing 1 | | | | |
| −1.5625000 | ZMAT1 | zinc finger, matrin-type 1 | 84460 | NM_001011657 | Hs.496512 | ENSG00000166432 |
| −1.4705882 | ZSWIM7 | zinc finger, SWIM-type | 125150 | NM_001042697 | Hs.593985 | ENSG00000214941 |
| | | containing 7 | | | | |
| 2.1700000 | ZMYM6NB | ZMYM6 neighbor | 100506144 | NM_001195156 | Hs.533986 | ENSG00000243749 |
| −2.3255814 | ZNRF3-AS1 | ZNRF3 antisense RNA 1 | 100874123 | NR_046851 | Hs.674708 | ENSG00000177993 |
| −1.9230769 | ZYG11A | zyg-11 family member A, cell | 440590 | NM_001004339 | Hs.658458 | ENSG00000203995 |
| | | cycle regulator | | | | |
| −3.2258065 | LOC101928243 | Non-annotated gene | 101928243 | | | |
| −3.1250000 | OLMALINC | Oligodendrocyte Maturation- | 90271 | NR_026762 | | ENSG00000235823 |
| | | Associated Long Intergenic | | | | |
| | | Non-Coding RNA | | | | |
| −2.9411765 | LOC100506385 | long intergenic non-protein | | NR_038885/ | | ENSG00000234380 |
| | | coding RNA 1426 | | NR_038886 | | |
| −2.6315789 | LOC400644 | long intergenic non-protein | | NR_104164 | | ENSG00000266554 |
| | | coding RNA 1443 | | | | |
| −2.4390244 | HECTD2-AS1 | HECTD2 antisense RNA 1 | 100188947 | NR_024467 | | |
| −2.3809524 | LINC01530 | long intergenic non-protein | 729975 | NR_034159 | | |
| | | coding RNA 1530 | | | | |
| −2.3255814 | TCAF2 | TRPM8 Channel Associated | 285966 | | | ENSG00000170379 |
| | | Factor 2 | | | | |
| −2.3255814 | LOC100130954 | long intergenic non-protein | | NR_034016/ | | |
| | | coding RNA 1502 | | NR_034017/ | | |
| | | | | NR109814 | | |
| −2.3255814 | RRP7B | Ribosomal RNA Processing 7 | 91695 | NR_002184 | | ENSG00000182841 |
| | | Homolog B, Pseudogene | | | | |
| −2.2222222 | P3H4 | Prolyl 3-Hydroxylase Family | 10609 | NM_006455 | | ENSG00000141696 |
| | | Member 4 | | | | |
| −2.1276596 | L3MBTL4-AS1 | L3MBTL4 antisense RNA 1 | 10192715 | | | ENSG00000264707 |
| −2.0408163 | SIRPG-AS1 | SIRPG antisense RNA 1 | 10192901 | NR_110090 | | ENSG00000237914 |
| −1.9607843 | RUNDC3A-AS1 | RUNDC3A antisense RNA 1 | 101926996 | NR_110802 | | ENSG00000267750 |
| −1.8518519 | SLC25A25-AS1 | SLC25A25 antisense RNA 1 | | NR_033374 | | |
| −1.7543860 | LINC01521 | long intergenic non-protein | 54944 | NR_120386 | | ENSG00000213888 |
| | | coding RNA 1521 | | | | |
| −1.6949153 | LOC100506747 | alpha-1,3-mannosyl- | 100506747 | NR_036557 | | ENSG00000234761 |
| | | glycoprotein 4-beta-N- | | | | |
| | | acetylglucosaminyltransferase- | | | | |
| | | like protein LOC641515 | | | | |
| −1.3698630 | RBSN | Rabenosyn, RAB Effector | 64145 | | | ENSG00000131381 |
| 2.1700000 | CPTP | ceramide-1-phosphate | 80772 | NM_001029885 | | ENSG00000224051 |
| | | transfer protein | | | | |

TABLE 17B
Molecular Mechanisms and Pathways Associated with Monocyte Subtype Genes

| UniqueID | Associated Pathways/Mechanism |
|---|---|
| ABCA9 | ABC transporters |
| ABCC9 | ABC transporters |
| RIPK1 | Acetylation and Deacetylation of RelA in The Nucleus, Ceramide Signaling Pathway, HIV- |
| | I Nef: negative effector of Fas and TNF, Induction of apoptosis through DR3 and DR4/5 |
| | Death Receptors, Keratinocyte Differentiation, MAPKinase Signaling Pathway, NF-kB |
| | Signaling Pathway, p38 MAPK Signaling Pathway, SODD/TNFR1 Signaling Pathway, |
| | TNF/Stress Related Signaling, TNFR1 Signaling Pathway, TNFR2 Signaling Pathway, |
| | Apoptosis, Cytosolic DNA-sensing pathway, Hepatitis C, RIG-I-like receptor signaling |
| | pathway, Toll-like receptor signaling pathway |
| RAC2 | Adherens junction, Axon guidance, B cell receptor signaling pathway, Chemokine |
| | signaling pathway, Colorectal cancer, Fc epsilon RI signaling pathway, Fc gamma R- |
| | mediated phagocytosis, Focal adhesion, Leukocyte transendothelial migration, MAPK |
| | signaling pathway, Natural killer cell mediated cytotoxicity, Pancreatic cancer, Pathways in |
| | cancer, Regulation of actin cytoskeleton, VEGF signaling pathway, Viral myocarditis, Wnt |
| | signaling pathway |
| YES1 | Adherens junction, Tight junction |
| GPLD1 | ADP-Ribosylation Factor, Glycosylphosphatidylinositol(GPI)-anchor biosynthesis |
| APOL1 | African trypanosomiasis |
| IDO1 | African trypanosomiasis, Metabolic pathways, Tryptophan metabolism |
| GLUD1 | Alanine, aspartate and glutamate metabolism, Arginine and proline metabolism, D- |
| | Glutamine and D-glutamate metabolism, Metabolic pathways, Nitrogen metabolism, |
| | Proximal tubule bicarbonate reclamation |
| ADSL | Alanine, aspartate and glutamate metabolism, Metabolic pathways, Purine metabolism |
| CAD | Alanine, aspartate and glutamate metabolism, Metabolic pathways, Pyrimidine metabolism |
| BMP7 | ALK in cardiac myocytes, Cytokine-cytokine receptor interaction, Hedgehog signaling |
| | pathway, TGF-beta signaling pathway |
| PLA2G4E | alpha-Linolenic acid metabolism, Arachidonic acid metabolism, Ether lipid metabolism, |
| | Fat digestion and absorption, Fc epsilon RI signaling pathway, Fc gamma R-mediated |
| | phagocytosis, Glycerophospholipid metabolism, GnRH signaling pathway, Linoleic acid |
| | metabolism, Long-term depression, MAPK signaling pathway, Metabolic pathways, |
| | Pancreatic secretion, Toxoplasmosis, Vascular smooth muscle contraction, VEGF signaling |
| | pathway |
| CYCS | Alzheimer's disease, Amyotrophic lateral sclerosis (ALS), Apoptosis, Colorectal cancer, |
| | Huntington's disease, p53 signaling pathway, Parkinson's disease, Pathways in cancer, |
| | Small cell lung cancer, Toxoplasmosis, Viral myocarditis |
| COX6B2 | Alzheimer's disease, Cardiac muscle contraction, Huntington's disease, Metabolic |
| | pathways, Oxidative phosphorylation, Parkinson's disease |
| COX8A | Alzheimer's disease, Cardiac muscle contraction, Huntington's disease, Metabolic |
| | pathways, Oxidative phosphorylation, Parkinson's disease |
| CYC1 | Alzheimer's disease, Cardiac muscle contraction, Huntington's disease, Metabolic |
| | pathways, Oxidative phosphorylation, Parkinson's disease |
| UQCR10 | Alzheimer's disease, Cardiac muscle contraction, Huntington's disease, Metabolic |
| | pathways, Oxidative phosphorylation, Parkinson's disease |
| ATP5B | Alzheimer's disease, Huntington's disease, Metabolic pathways, Oxidative phosphorylation, |
| | Parkinson's disease |
| ATP5D | Alzheimer's disease, Huntington's disease, Metabolic pathways, Oxidative phosphorylation, |
| | Parkinson's disease |
| ATP5G2 | Alzheimer's disease, Huntington's disease, Metabolic pathways, Oxidative phosphorylation, |
| | Parkinson's disease |
| NDUFA7 | Alzheimer's disease, Huntington's disease, Metabolic pathways, Oxidative phosphorylation, |
| | Parkinson's disease |
| NDUFB6 | Alzheimer's disease, Huntington's disease, Metabolic pathways, Oxidative phosphorylation, |
| | Parkinson's disease |
| NDUFB7 | Alzheimer's disease, Huntington's disease, Metabolic pathways, Oxidative phosphorylation, |
| | Parkinson's disease |
| NDUFB8 | Alzheimer's disease, Huntington's disease, Metabolic pathways, Oxidative phosphorylation, |
| | Parkinson's disease |
| NDUFC1 | Alzheimer's disease, Huntington's disease, Metabolic pathways, Oxidative phosphorylation, |
| | Parkinson's disease |
| NDUFS7 | Alzheimer's disease, Huntington's disease, Metabolic pathways, Oxidative phosphorylation, |
| | Parkinson's disease |
| NDUFB11 | Alzheimer's disease, Huntington's disease, Oxidative phosphorylation, Parkinson's disease |
| GMPPA | Amino sugar and nucleotide sugar metabolism, Fructose and mannose metabolism, |
| | Metabolic pathways |
| GNE | Amino sugar and nucleotide sugar metabolism, Metabolic pathways |
| AARS2 | Aminoacyl-tRNA biosynthesis |
| MTFMT | Aminoacyl-tRNA biosynthesis, One carbon pool by folate |
| RAB5C | Amoebiasis, Endocytosis, Phagosome, Vasopressin-regulated water reabsorption |
| MAP2K2 | Angiotensin II mediated activation of JNK Pathway via Pyk2 dependent signaling, Anthrax |
| | Toxin Mechanism of Action, Bioactive Peptide Induced Signaling Pathway, Erk1/Erk2 |
| | Mapk Signaling pathway, fMLP induced chemokine gene expression in HMC-1 cells, |
| | Human Cytomegalovirus and Map Kinase Pathways, Integrin Signaling Pathway, Links |
| | between Pyk2 and Map Kinases, MAPKinase Signaling Pathway, Phosphorylation of |
| | MEK1 by cdk5/p35 down regulates the MAP kinase pathway, Role of b-arrestins in the |
| | activation and targeting of MAP kinases, Role of MAL in Rho-Mediated Activation of |
| | SRF, Roles of b-arrestin-dependent Recruitment of Src Kinases in GPCR Signaling, |
| | Signaling of Hepatocyte Growth Factor Receptor, Acute myeloid leukemia, B cell receptor |
| | signaling pathway, Bladder cancer, Chronic myeloid leukemia, Endometrial cancer, ErbB |
| | signaling pathway, Fc epsilon RI signaling pathway, Gap junction, Glioma, GnRH |
| | signaling pathway, Insulin signaling pathway, Long-term depression, Long-term |
| | potentiation |
| LGMN | Antigen processing and presentation, Lysosome |
| PSMB5 | Antigen Processing and Presentation, Proteasome |
| DFFA | Apoptotic DNA fragmentation and tissue homeostasis, Caspase Cascade in Apoptosis, FAS |
| | signaling pathway (CD95), Granzyme A mediated Apoptosis Pathway, HIV-I Nef: |
| | negative effector of Fas and TNF, Induction of apoptosis through DR3 and DR4/5 Death |
| | Receptors, Role of Mitochondria in Apoptotic Signaling, TNFR1 Signaling Pathway, |
| | Apoptosis |
| ALDH2 | Arginine and proline metabolism, Ascorbate and aldarate metabolism, beta-Alanine |
| | metabolism, Fatty acid degradation, Glycerolipid metabolism, Glycolysis/ |
| | Gluconeogenesis, Histidine metabolism, Lysine degradation, Metabolic pathways, Pentose |
| | and glucuronate interconversions, Propanoate metabolism, Pyruvate metabolism, |
| | Tryptophan metabolism, Valine, leucine and isoleucine degradation |
| DSG2 | Arrhythmogenic right ventricular cardiomyopathy (ARVC) |
| TBXAS1 | Aspirin Blocks Signaling Pathway Involved in Platelet Activation, Eicosanoid Metabolism, |
| | Arachidonic acid metabolism, Metabolic pathways |
| RGS3 | Axon guidance |
| SEMA4A | Axon guidance |
| SEMA4B | Axon guidance |
| XIAP | B Cell Survival Pathway, Caspase Cascade in Apoptosis, HIV-I Nef: negative effector of |
| | Fas and TNF, Induction of apoptosis through DR3 and DR4/5 Death Receptors, Role of |
| | Mitochondria in Apoptotic Signaling, Apoptosis, Focal adhesion, NOD-like receptor |
| | signaling pathway, Pathways in cancer, Small cell lung cancer, Toxoplasmosis, Ubiquitin |
| | mediated proteolysis |
| RHOG | Bacterial invasion of epithelial cells, Shigellosis |
| GTF2H4 | Basal transcription factors, Nucleotide excision repair |
| POLD4 | Base excision repair, DNA replication, Homologous recombination, Metabolic pathways, |
| | Mismatch repair, Nucleotide excision repair, Purine metabolism, Pyrimidine metabolism |
| POLE3 | Base excision repair, DNA replication, Metabolic pathways, Nucleotide excision repair, |
| | Purine metabolism, Pyrimidine metabolism |
| PPP3CB | BCR Signaling Pathway, Control of skeletal myogenesis by HDAC & calcium/calmodulin- |
| | dependent kinase (CaMK), Effects of calcineurin in Keratinocyte Differentiation, |
| | Endocytotic role of NDK, Phosphins and Dynamin, Fc Epsilon Receptor I Signaling in |
| | Mast Cells, fMLP induced chemokine gene expression in HMC-1 cells, Neuropeptides VIP |
| | and PACAP inhibit the apoptosis of activated T cells, NFAT and Hypertrophy of the heart |
| | (Transcription in the broken heart), Nitric Oxide Signaling Pathway, Regulation of PGC-1a, |
| | Role of MEF2D in T-cell Apoptosis, Signaling Pathway from G-Protein Families, T Cell |
| | Receptor Signaling Pathway, Alzheimer's disease, Amyotrophic lateral sclerosis (ALS), |
| | Apoptosis, Axon guidance, B cell receptor signaling pathway, Calcium signaling pathway, |
| | Long-term potentiation, MAPK signaling pathway, Natural killer cell mediated |
| | cytotoxicity, Oocyte meiosis, Osteoclast differentiation, T cell receptor signaling pathway, |
| | VEGF signaling pathway, Wnt signaling pathway |
| TECR | Biosynthesis of unsaturated fatty acids |
| SCD5 | Biosynthesis of unsaturated fatty acids, PPAR signaling pathway |
| L2HGDH | Butanoate metabolism |
| MYLK3 | Calcium signaling pathway, Focal adhesion, Gastric acid secretion, Regulation of actin |
| | cytoskeleton, Vascular smooth muscle contraction |
| CHRM3 | Calcium signaling pathway, Gastric acid secretion, Neuroactive ligand-receptor interaction, |
| | Pancreatic secretion, Regulation of actin cytoskeleton, Salivary secretion |
| SLC25A5 | Calcium signaling pathway, Huntington's disease, Parkinson's disease |
| ADRA1A | Calcium signaling pathway, Neuroactive ligand-receptor interaction, Salivary secretion, |
| | Vascular smooth muscle contraction |
| PHB2 | CARM1 and Regulation of the Estrogen Receptor |
| ORC4 | CDK Regulation of DNA Replication, Cell cycle |
| CLDN15 | Cell adhesion molecules (CAMs), Hepatitis C, Leukocyte transendothelial migration, Tight |
| | junction |
| CLDN19 | Cell adhesion molecules (CAMs), Hepatitis C, Leukocyte transendothelial migration, Tight |
| | junction |
| PPP2CB | Chagas disease (American trypanosomiasis), Hepatitis C, Long-term depression, mRNA |
| | surveillance pathway, Oocyte meiosis, TGF-beta signaling pathway, Tight junction, Wnt |
| | signaling pathway |
| GNB2 | Chemokine signaling pathway |
| GNG4 | Chemokine signaling pathway |
| CCR6 | Chemokine signaling pathway, Cytokine-cytokine receptor interaction |
| GAB2 | Chronic myeloid leukemia, Fc epsilon RI signaling pathway, Fc gamma R-mediated |
| | phagocytosis, Osteoclast differentiation |
| PER2 | Circadian rhythm |
| ACO2 | Citrate cycle (TCA cycle), Glyoxylate and dicarboxylate metabolism, Metabolic pathways |
| MDH2 | Citrate cycle (TCA cycle), Glyoxylate and dicarboxylate metabolism, Metabolic pathways, |
| | Pyruvate metabolism |
| CRK | CXCR4 Signaling Pathway, Signaling of Hepatocyte Growth Factor Receptor, Bacterial |
| | invasion of epithelial cells, Chemokine signaling pathway, Chronic myeloid leukemia, |
| | ErbB signaling pathway, Fc gamma R-mediated phagocytosis, Focal adhesion, Insulin |
| | signaling pathway, MAPK signaling pathway, Neurotrophin signaling pathway, Pathways |
| | in cancer, Regulation of actin cytoskeleton, Renal cell carcinoma, Shigellosis |
| SHMT2 | Cyanoamino acid metabolism, Glycine, serine and threonine metabolism, Metabolic |
| | pathways, One carbon pool by folate |
| TAT | Cysteine and methionine metabolism, Metabolic pathways, Phenylalanine metabolism, |
| | Phenylalanine, tyrosine and tryptophan biosynthesis, Tyrosine metabolism, Ubiquinone and |
| | other terpenoid-quinone biosynthesis |
| CRLF2 | Cytokine-cytokine receptor interaction, Jak-STAT signaling pathway |
| IFNLR1 | Cytokine-cytokine receptor interaction, Jak-STAT signaling pathway |
| OSMR | Cytokine-cytokine receptor interaction, Jak-STAT signaling pathway |
| PYCARD | Cytosolic DNA-sensing pathway, NOD-like receptor signaling pathway |
| GREB1 | Downregulated of MTA-3 in ER-negative Breast Tumors |
| GSTM3 | Drug metabolism - cytochrome P450, Glutathione metabolism, Metabolism of xenobiotics |
| | by cytochrome P450 |
| MGST1 | Drug metabolism - cytochrome P450, Glutathione metabolism, Metabolism of xenobiotics |
| | by cytochrome P450 |
| CDA | Drug metabolism - other enzymes, Metabolic pathways, Pyrimidine metabolism |
| CSNK2A1 | EGF Signaling Pathway, EPO Signaling Pathway, IGF-1 Signaling Pathway, IL 2 signaling |
| | pathway, IL 6 signaling pathway, Insulin Signaling Pathway, Lissencephaly gene (LIS1) in |
| | neuronal migration and development, Nerve growth factor pathway (NGF), PDGF |
| | Signaling Pathway, TPO Signaling Pathway, WNT Signaling Pathway, Adherens junction, |
| | Ribosome biogenesis in eukaryotes, Tight junction, Wnt signaling pathway |
| SDHB | Electron Transport Reaction in Mitochondria, Alzheimer's disease, Citrate cycle (TCA |
| | cycle), Huntington's disease, Metabolic pathways, Oxidative phosphorylation, Parkinson's |
| | disease |
| CHMP1B | Endocytosis |
| CHMP3 | Endocytosis |
| PDCD6IP | Endocytosis |
| RAB11FIP4 | Endocytosis |
| VPS28 | Endocytosis |
| AP2B1 | Endocytosis, Huntington's disease |
| FGFR2 | Endocytosis, MAPK signaling pathway, Pathways in cancer, Prostate cancer, Regulation of |
| | actin cytoskeleton |
| PARD6G | Endocytosis, Tight junction |
| STAT5B | EPO Signaling Pathway, Growth Hormone Signaling Pathway, IL 2 signaling pathway, IL |
| | 3 signaling pathway, IL-2 Receptor Beta Chain in T cell Activation, IL22 Soluble Receptor |
| | Signaling Pathway, IL-7 Signal Transduction, Inhibition of Cellular Proliferation by |
| | Gleevec, Mechanism of Gene Regulation by Peroxisome Proliferators via PPARa(alpha), |
| | TPO Signaling Pathway, Acute myeloid leukemia, Chemokine signaling pathway, Chronic |
| | myeloid leukemia, ErbB signaling pathway, Jak-STAT signaling pathway, Pathways in |
| | cancer |
| GSN | Erk and PI-3 Kinase Are Necessary for Collagen Binding in Corneal Epithelia, HIV-I Nef: |
| | negative effector of Fas and TNF, Rho cell motility signaling pathway, Fc gamma R- |
| | mediated phagocytosis, Regulation of actin cytoskeleton |
| EIF5 | Eukaryotic protein translation, Regulation of eIF2, RNA transport |
| CFLAR | FAS signaling pathway (CD95), HIV-I Nef: negative effector of Fas and TNF, IL-2 |
| | Receptor Beta Chain in T cell Activation, Induction of apoptosis through DR3 and DR4/5 |
| | Death Receptors, Apoptosis, Chagas disease (American trypanosomiasis) |
| OLAH | Fatty acid biosynthesis, Metabolic pathways |
| ACADSB | Fatty acid degradation, Metabolic pathways, Valine, leucine and isoleucine degradation |
| AKR1B1 | Fructose and mannose metabolism, Galactose metabolism, Glycerolipid metabolism, |
| | Metabolic pathways, Pentose and glucuronate interconversions, Pyruvate metabolism |
| TUBB | Gap junction, Pathogenic Escherichia coli infection, Phagosome |
| SLC9A4 | Gastric acid secretion |
| GPX4 | Glutathione metabolism |
| LYPLA2 | Glycerophospholipid metabolism |
| CDIPT | Glycerophospholipid metabolism, Inositol phosphate metabolism, Metabolic pathways, |
| | Phosphatidylinositol signaling system |
| PISD | Glycerophospholipid metabolism, Metabolic pathways |
| B3GALT6 | Glycosaminoglycan biosynthesis - chondroitin sulfate/dermatan sulfate, |
| | Glycosaminoglycan biosynthesis - heparan sulfate/heparin, Metabolic pathways |
| CHST6 | Glycosaminoglycan biosynthesis - keratan sulfate |
| FUT1 | Glycosphingolipid biosynthesis - globo series, Glycosphingolipid biosynthesis - lacto and |
| | neolacto series, Metabolic pathways |
| FUT2 | Glycosphingolipid biosynthesis - globo series, Glycosphingolipid biosynthesis - lacto and |
| | neolacto series, Metabolic pathways |
| NAGA | Glycosphingolipid biosynthesis - globo series, Lysosome |
| PIGY | Glycosylphosphatidylinositol(GPI)-anchor biosynthesis, Metabolic pathways |
| CD24 | Hematopoietic cell lineage |
| METTL2B | Histidine metabolism, Tyrosine metabolism |
| MUS81 | Homologous recombination |
| XRCC2 | Homologous recombination |
| DNAL1 | Huntington's disease |
| POLR2D | Huntington's disease, Metabolic pathways, Purine metabolism, Pyrimidine metabolism, |
| | RNA polymerase |
| DCTN2 | Huntington's disease, Vasopressin-regulated water reabsorption |
| NQO1 | Hypoxia and p53 in the Cardiovascular system |
| IKZF3 | IL-2 Receptor Beta Chain in T cell Activation |
| CRKL | IL-2 Receptor Beta Chain in T cell Activation, Inhibition of Cellular Proliferation by |
| | Gleevec, Integrin Signaling Pathway, Links between Pyk2 and Map Kinases, Signaling of |
| | Hepatocyte Growth Factor Receptor, Bacterial invasion of epithelial cells, Chemokine |
| | signaling pathway, Chronic myeloid leukemia, ErbB signaling pathway, Fc gamma R- |
| | mediated phagocytosis, Focal adhesion, Insulin signaling pathway, MAPK signaling |
| | pathway, Neurotrophin signaling pathway, Pathways in cancer, Regulation of actin |
| | cytoskeleton, Renal cell carcinoma, Shigellosis |
| INPP5E | Inositol phosphate metabolism, Metabolic pathways, Phosphatidylinositol signaling system |
| ALDH6A1 | Inositol phosphate metabolism, Metabolic pathways, Propanoate metabolism, Valine, |
| | leucine and isoleucine degradation |
| GYS1 | Insulin signaling pathway, Starch and sucrose metabolism |
| SPRED1 | Jak-STAT signaling pathway |
| MAPK13 | Keratinocyte Differentiation, MAPKinase Signaling Pathway, Stathmin and breast cancer |
| | resistance to antimicrotubule agents, Amyotrophic lateral sclerosis (ALS), Chagas disease |
| | (American trypanosomiasis), Epithelial cell signaling in Helicobacter pylori infection, Fc |
| | epsilon RI signaling pathway, GnRH signaling pathway, Hepatitis C, Leishmaniasis, |
| | Leukocyte transendothelial migration, MAPK signaling pathway, Neurotrophin signaling |
| | pathway, NOD-like receptor signaling pathway, Osteoclast differentiation, Progesterone- |
| | mediated oocyte maturation, RIG-I-like receptor signaling pathway, Shigellosis, T cell |
| | receptor signaling pathway, Toll-like receptor signaling pathway, Toxoplasmosis, VEGF |
| | signaling pathway |
| GALK1 | Leloir pathway of galactose metabolism, Amino sugar and nucleotide sugar metabolism, |
| | Galactose metabolism, Metabolic pathways |
| AASS | Lysine biosynthesis, Lysine degradation, Metabolic pathways |
| SETMAR | Lysine degradation |
| SUV420H2 | Lysine degradation |
| AP1M1 | Lysosome |
| AP1S3 | Lysosome |
| AP4S1 | Lysosome |
| CD164 | Lysosome |
| ENTPD4 | Lysosome, Purine metabolism, Pyrimidine metabolism |
| ACP2 | Lysosome, Riboflavin metabolism |
| SLC25A11 | Malate-aspartate shuttle, Shuttle for transfer of acetyl groups from mitochondria to the |
| | cytosol |
| MDH1 | Malate-aspartate shuttle, Shuttle for transfer of acetyl groups from mitochondria to the |
| | cytosol, Citrate cycle (TCA cycle), Glyoxylate and dicarboxylate metabolism, Metabolic |
| | pathways, Proximal tubule bicarbonate reclamation, Pyruvate metabolism |
| DUSP7 | MAPK signaling pathway |
| FGF5 | MAPK signaling pathway, Melanoma, Pathways in cancer, Regulation of actin |
| | cytoskeleton |
| MAP2K5 | MAPKinase Signaling Pathway, Gap junction, MAPK signaling pathway, Neurotrophin |
| | signaling pathway |
| RPS6KA4 | MAPKinase Signaling Pathway, MAPK signaling pathway, Neurotrophin signaling |
| | pathway |
| MAP3K9 | MAPKinase Signaling Pathway, p38 MAPK Signaling Pathway |
| IAPP | Maturity onset diabetes of the young |
| APOA2 | Mechanism of Gene Regulation by Peroxisome Proliferators via PPARa(alpha), PPAR |
| | signaling pathway |
| TGDS | Metabolic pathways |
| B3GNT6 | Metabolic pathways, Mucin type O-Glycan biosynthesis |
| NMNAT1 | Metabolic pathways, Nicotinate and nicotinamide metabolism |
| PGLS | Metabolic pathways, Pentose phosphate pathway |
| H6PD | Metabolic pathways, Pentose phosphate pathway |
| CYP51A1 | Metabolic pathways, Steroid biosynthesis |
| EBP | Metabolic pathways, Steroid biosynthesis |
| COMT | Metabolic pathways, Steroid hormone biosynthesis, Tyrosine metabolism |
| CSAD | Metabolic pathways, Taurine and hypotaurine metabolism |
| BCKDHA | Metabolic pathways, Valine, leucine and isoleucine degradation |
| PNN | mRNA surveillance pathway, RNA transport |
| ULK3 | mTOR signaling pathway, Regulation of autophagy |
| GSTP1 | Multi-Drug Resistance Factors, Drug metabolism - cytochrome P450, Glutathione |
| | metabolism, Metabolism of xenobiotics by cytochrome P450, Pathways in cancer, Prostate |
| | cancer |
| HCST | Natural killer cell mediated cytotoxicity |
| TRPV1 | Neuroactive ligand-receptor interaction |
| TSPO | Neuroactive ligand-receptor interaction |
| VIPR1 | Neuroactive ligand-receptor interaction |
| UBE2D1 | Neuroregulin receptor degredation protein-1 Controls ErbB3 receptor recycling, Protein |
| | processing in endoplasmic reticulum, Ubiquitin mediated proteolysis |
| CA5B | Nitrogen metabolism |
| MFNG | Notch signaling pathway, Other types of O-glycan biosynthesis |
| DDB1 | Nucleotide excision repair, Ubiquitin mediated proteolysis |
| OR11A1 | Olfactory transduction |
| SGOL1 | Oocyte meiosis |
| LILRA2 | Osteoclast differentiation |
| SQSTM1 | Osteoclast differentiation |
| MDM4 | p53 signaling pathway |
| TP53AIP1 | p53 signaling pathway |
| PARK7 | Parkinson's disease |
| PARK2 | Parkinson's disease, Protein processing in endoplasmic reticulum, Ubiquitin mediated |
| | proteolysis |
| ECH1 | Peroxisome |
| MPV17L | Peroxisome |
| PXMP4 | Peroxisome |
| CORO1A | Phagosome |
| DBI | PPAR signaling pathway |
| SLC27A1 | PPAR signaling pathway |
| PSMD14 | Proteasome |
| SLC15A1 | Protein digestion and absorption |
| SPCS1 | Protein export |
| SRP9 | Protein export |
| SSR2 | Protein processing in endoplasmic reticulum |
| PDE4C | Purine metabolism |
| KLRD1 | Ras-Independent pathway in NK cell-mediated cytotoxicity, Antigen processing and |
| | presentation, Graft-versus-host disease, Natural killer cell mediated cytotoxicity |
| OPHN1 | Rho cell motility signaling pathway |
| RPL18A | Ribosome |
| RPL28 | Ribosome |
| RPL41 | Ribosome |
| RPS12 | Ribosome |
| RPLP1 | Ribosome |
| POP7 | Ribosome biogenesis in eukaryotes, RNA transport |
| DIS3 | RNA degradation |
| AAAS | RNA transport |
| GEMIN8 | RNA transport |
| NUP133 | RNA transport |
| PHAX | RNA transport |
| RAD1 | Role of BRCA1, BRCA2 and ATR in Cancer Susceptibility |
| TREX1 | Role of BRCA1, BRCA2 and ATR in Cancer Susceptibility, Cytosolic DNA-sensing |
| | pathway |
| DIABLO | Role of Mitochondria in Apoptotic Signaling |
| CHRNB1 | Role of nicotinic acetylcholine receptors in the regulation of apoptosis, Neuroactive ligand- |
| | receptor interaction |
| INMT | Selenocompound metabolism, Tryptophan metabolism |
| SLC25A1 | Shuttle for transfer of acetyl groups from mitochondria to the cytosol |
| GOSR1 | SNARE interactions in vesicular transport |
| DYRK1B | Sonic Hedgehog (Shh) Pathway |
| CTNNBL1 | Spliceosome |
| CCDC12 | Spliceosome |
| HNRNPA1L2 | Spliceosome |
| PRPF3 | Spliceosome |
| PRPF38B | Spliceosome |
| PRPF8 | Spliceosome |
| RBM25 | Spliceosome |
| SRSF10 | Spliceosome |
| SRSF4 | Spliceosome |
| SF3A2 | Spliceosome |
| TCERG1 | Spliceosome |
| FUT6 | Steps in the Glycosylation of Mammalian N-linked Oligosaccharides, Glycosphingolipid |
| | biosynthesis - lacto and neolacto series, Metabolic pathways |
| MOCS3 | Sulfur relay system |
| H2AFJ | Systemic lupus erythematosus |
| H2AFX | Systemic lupus erythematosus |
| HIST1H2AC | Systemic lupus erythematosus |
| HIST1H3H | Systemic lupus erythematosus |
| CAT | The IGF-1 Receptor and Longevity, Amyotrophic lateral sclerosis (ALS), Metabolic |
| | pathways, Peroxisome, Tryptophan metabolism |
| Yyl | The PRC2 Complex Sets Long-term Gene Silencing Through Modification of Histone Tails |
| TOLLIP | Toll-like receptor signaling pathway |
| UBE3C | Ubiquitin mediated proteolysis |
| KDELR1 | Vibrio cholerae infection |
| KDELR2 | Vibrio cholerae infection |
| CXADR | Viral myocarditis |
| PDE6A | Visual Signal Transduction, Phototransduction, Purine metabolism |
| SLC19A1 | Vitamin digestion and absorption |

TABLE 18
Anti-TL1A and Anti-DR3 Antibody Sequences

| SEQ ID | | |
|---|---|---|
| NO | Identifier | Amino Acid Sequence |
| 209 | HCDR1 | GFTFSTYG |
| 210 | HCDR2 | ISGTGRTT |
| 211 | HCDR3 | TKERGDYYYG VFDY |
| 212 | LCDR1 | QTISSW |
| 213 | LCDR2 | AAS |
| 214 | LCDR3 | QQYHRSWT |
| 215 | HC | EVQLLESGGG LVQPGKSLRL SCAVSGFTFS TYGMNWVRQA |
| | Variable | PGKGLEWVSS |
| | | ISGTGRTTYH ADSVQGRFTV SRDNSKNILY LQMNSLRADD |
| | | TAVYFCTKER |
| | | GDYYYGVFDY WGQGTLVTVS S |
| 216 | LC | DIQMTQSPST LSASVGDRVT ITCRASQTIS SWLAWYQQTP |
| | Variable | EKAPKLLIYA |
| | | ASNLQSGVPS RFSGSGSGTE FTLTISSLQP DDFATYYCQQ |
| | | YHRSWTFGQG |
| | | TKVEIT |
| 217 | HCDR1 | GFTFSSYW |
| 218 | HCDR2 | IKEDGSEK |
| 219 | HCDR3 | AREDYDSYYK YGMDV |
| 220 | LCDR1 | QSILYSSNNK NY |
| 221 | LCDR2 | WAS |
| 222 | LCDR3 | QQYYSTPFT |
| 223 | HC | EVOLVESGGG LVQPGGSLRL SCAVSGFTFS SYWMSWVRQA |
| | Variable | PGKGLEWVAN |
| | | IKEDGSEKNY VDSVKGRFTL SSDNAKNSLY LQMNSLRAED |
| | | TAVYYCARED |
| | | YDSYYKYGMD VWGQGTAVIV SS |
| 224 | LC | DIVMTQSPDS LAVSLGERAT INCKSSQSIL YSSNNKNYLA |
| | Variable | WYQQKPGQPP |
| | | KLLIYWASTR ESGVPDRFSG SGSGTDFTLT ISSLQAEDVS |
| | | VYYCQQYYST |
| | | PFTFGPGTKV DIK |
| 225 | HCDR1 | GGSFTGFY |
| 226 | HCDR2 | INHRGNT |
| 227 | HCDR3 | ASPFYDFWSG SDY |
| 228 | LCDR1 | QSLVHSDGNT Y |
| 229 | LCDR2 | KIS |
| 230 | LCDR3 | MQATQFPLT |
| 231 | HC | QVQLQQWGAG LLKPSETLSL TCAVYGGSFT GFYWSWIRQP |
| | Variable | PGKGLEWIGE |
| | | INHRGNTNYN PSLKSRVTMS VDTSKNQFSL NMISVTAADT |
| | | AMYFCASPFY |
| | | DFWSGSDYWG QGTLVTVSS |
| 232 | LC | DIMLTQTPLT SPVTLGQPAS ISCKSSQSLV HSDGNTYLSW |
| | Variable | LQQRPGQPPR |
| | | LLFYKISNRF SGVPDRFSGS GAGTDFTLKI SRVEAEDVGV |
| | | YYCMQATQFP |
| | | LTFGGGTKVE IK |
| 233 | HCDR1 | GY(X1)F(X2)(X3)YGIS; X1 = P, S, D, Q, N; X2 = T, R; X3 = N, T, Y, H |
| 234 | HCDR2 | WIS(X1)YNG(X2)(X3)(X4)YA(X5)(X6)(X7)QG; X1 = T, P, S, A; X2 = N, G, |
| | | V, K, A; X3 = T, K; X4 = H, N; X5 = Q, R; X6 = K, M; X7 = L, H |
| 235 | HCDR3 | ENYYGSG(X1)(X2)R GGMD(X3); X1 = S, A; X2 = Y, P; X3 = V, A, G |
| 236 | HCDR1 | GYDFTYYGIS |
| 237 | HCDR2 | WISTYNGNTH YARMLQG |
| 238 | HCDR3 | ENYYGSGAYR GGMDV |
| 239 | LCDR1 | RASQSVSSYL A |
| 240 | LCDR2 | DASNRAT |
| 241 | LCDR3 | QQRSNWPWT |
| 242 | HC | QVQLVQSGAE VKKPGASVKV SCKASGYDFT YYGISWVRQA |
| | Variable | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSS |
| 243 | LC | EIVLTQSPAT LSLSPGERAT LSCRASQSVS SYLAWYQQKP |
| | Variable | GQAPRLLIYD |
| | | ASNRATGIPA RFSGSGSGTD FTLTISSLEP EDFAVYYCQQ |
| | | RSNWPWTFGQ |
| | | GTKVEIK |
| 244 | HC | QVQLVQSGAE VKKPGASVKV SCKASGYDFT YYGISWVRQA |
| | | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSSASTKGP SVFPLAPSSK |
| | | STSGGTAALG |
| | | CLVKDYFPEP VTVSWNSGAL TSGVHTFPAV LQSSGLYSLS |
| | | SVVTVPSSSL |
| | | GTQTYICNVN HKPSNTKVDK KVEPKSCDKT HTCPPCPAPE |
| | | AAGAPSVFLF |
| | | PPKPKDTLMI SRTPEVTCVV VDVSHEDPEV KFNWYVDGVE |
| | | VHNAKTKPRE |
| | | EQYNSTYRVV SVLTVLHQDW LNGKEYKCKV SNKALPAPIE |
| | | KTISKAKGQP |
| | | REPQVYTLPP SREEMTKNQV SLTCLVKGFY PSDIAVEWES |
| | | NGQPENNYKT |
| | | TPPVLDSDGS FFLYSKLTVD KSRWQQGNVF SCSVMHEALH |
| | | NHYTQKSLSL |
| | | SPG |
| 245 | LC | EIVLTQSPAT LSLSPGERAT LSCRASQSVS SYLAWYQQKP |
| | | GQAPRLLIYD |
| | | ASNRATGIPA RFSGSGSGTD FTLTISSLEP EDFAVYYCQQ |
| | | RSNWPWTFGQ |
| | | GTKVEIKRTV AAPSVFIFPP SDEQLKSGTA SVVCLLNNFY |
| | | PREAKVQWKV |
| | | DNALQSGNSQ ESVTEQDSKD STYSLSSTLT LSKADYEKHK |
| | | VYACEVTHQG |
| | | LSSPVTKSFN RGEC |
| 246 | HCDR1 | SRSYYWG |
| 247 | HCDR2 | SIYYNGRTYY NPSLKS |
| 248 | HCDR3 | EDYGDYGAFD I |
| 249 | LCDR1 | RASQGISSAL A |
| 250 | LCDR2 | DASSLES |
| 251 | LCDR3 | QQFNSYPLT |
| 252 | HC | QLQLQESGPG LVKPSETLSL TCTVSGGSIS SRSYYWGWIR |
| | Variable | QPPGKGLEWI |
| | | GSIYYNGRTY YNPSLKSRVT ISVDTSKNQF SLKLSSVTAA |
| | | DTAVYYCARE |
| | | DYGDYGAFDI WGQGTMVTVS S |
| 253 | LC | AIQLTQSPSS LSASVGDRVT ITCRASQGIS SALAWYQQKP |
| | Variable | GKAPKLLIYD |
| | | ASSLESGVPS RFSGSGSGTD FTLTISSLQP EDFATYYCQQ |
| | | FNSYPLTFGG |
| | | GTKVEIK |
| 254 | HCDR1 | TSNMGVV |
| 255 | HCDR2 | HILWDDREYSNPALKS |
| 256 | HCDR3 | MSRNYYGSSYVMDY |
| 257 | LCDR1 | SASSSVNYMH |
| 258 | LCDR2 | STSNLAS |
| 259 | LCDR3 | HQWNNYGT |
| 260 | HC | QVTLKESGPALVKPTQTLTLTCTFSGFSLSTSNMGVVWIRQPPGKALEW |
| | Variable | LAHILWDD |
| | | REYSNPALKSRLTISKDTSKNQVVLTMTNMDPVDTATYYCARMSRNY |
| | | YGSSYVMD YWGQGTLVTVSS |
| 261 | LC | DIQLTQSPSFLSASVGDRVTITCSASSSVNYMHWYQQKPGKAPKLLIYS |
| | Variable | TSNLASGVP |
| | | SRFSGSGSGTEFTLTISSLQPEDFATYYCHQWNNYGTFGQGTKVEIKR |
| 262 | HCDR1 | LYGMN |
| 263 | HCDR1 | NYGMN |
| 264 | HCDR2 | WINTYTGEPTYADDFKG |
| 265 | HCDR3 | DTAMDYAMAY |
| 266 | HCDR3 | DYGKYGDYYAMDY |
| 267 | LCDR1 | KSSQNIVHSDGNTYLE |
| 268 | LCDR1 | RSSQSIVHSNGNTYLD |
| 269 | LCDR2 | KVSNRFS |
| 270 | LCDR3 | FOGSHVPLT |
| 271 | HC | QVQLVQSGSELKKPGASVKVSCKASGYTFTLYGMNWVRQAPGQGLE |
| | Variable | WMG |
| | | WINTYTGEPTYADDFKGRFVFSLDTSVSTAYLQISSLKAEDTAVYYCAR |
| | | DTAMDYAMAYWGQGTLVTVSS |
| 272 | HC | QVQLVQSGSELKKPGASVKVSCKASGYTFTLYGMNWVKQAPGKGLK |
| | Variable | WMG |
| | | WINTYTGEPTYADDFKGRFVFSLDTSVSTAYLQISSLKAEDTAVYFCAR |
| | | DTAMDYAMAYWGQGTLVTVSS |
| 273 | HC | QVQLVQSGSELKKPGASVKVSCKASGYTFTNYGMNWVRQAPGQGLE |
| | Variable | WMG |
| | | WINTYTGEPTYADDFKGRFVFSLDTSVSTAYLQISSLKAEDTAVYYCAR |
| | | DYGKYGDYYAMDYWGQGTLVTVSS |
| 274 | HC | QVQLVQSGSELKKPGASVKVSCKASGYTFTNYGMNWVRQAPGKGLK |
| | Variable | WMG |
| | | WINTYTGEPTY ADDFKGRFVFSLDTSVSTAYLQISSLKAEDTAVYFCAR |
| | | DYGKYGDYYAMDYWGQGTLVTVSS |
| 275 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 276 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 277 | LC | DVVMTQTPLSLPVTPGEPASISCKSSQNIVHSDGNTYLEWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 278 | LC | DVVMTQTPLSLPVSLGDQASISCKSSQNIVHSDGNTYLEWYLQKPGQSP |
| | Variable | KVLIYKVSNRFSGVPDRFSGSGSGTDFTLKISRVEAEDLGVYYCFQGSH |
| | | VPLTFGGGTKVEIKR |
| 279 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 280 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 281 | LC | DVVMTQTPLSLPVTPGEPASISCRSSQSIVHSNGNTYLDWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 282 | LC | DVVMTQTPLSLPVSLGDQASISCRSSQSIVHSNGNTYLDWYLQKPGQSP |
| | Variable | KVLIYKVSNRFSGVPDRFSGSGSGTDFTLKINRVEAEDLGVYFCFQGSH |
| | | VPLTFGGGTKLEIKR |
| 283 | HCDR1 | GYTFTSSWMH |
| 284 | HCDR2 | IHPNSGGT |
| 285 | HCDR3 | ARGDYYGYVS WFAY |
| 286 | LCDR1 | QNINVL |
| 287 | LCDR2 | KAS |
| 288 | LCDR3 | QQGQSYPYT |
| 289 | HC | QVQLQQPGSV LVRPGASVKV SCKASGYTFT SSWMHWAKQR |
| | Variable | PGQGLEWIGE |
| | | IHPNSGGTNY NEKFKGKATV DTSSSTAYVD LSSLTSEDSA |
| | | VYYCARGDYY |
| | | GYVSWFAYWG QGTLVTVSS |
| 290 | HC | QVQLVQSGAE VKKPGASVKV SCKASGYTFT SSWMHWARQA |
| | Variable | PGQGLEWIGE |
| | | IHPNSGGTNY AQKFQGRATL TVDTSSSTAY MELSRLRSDD |
| | | TAVYYCARGD |
| | | YYGYVSWFAY WGQGTLVTVS S |
| 291 | HC | QVQLVQSGAE VKKPGASVKV SCKASGYTFT SSWMHWARQA |
| | Variable | PGQGLEWIGE |
| | | IHPNSGGTNY AQKFQGRATM TVDTSISTAY MELSRLRSDD |
| | | TAVYYCARGD |
| | | YYGYVSWFAY WGQGTLVTVS S |
| 292 | HC | QVQLVQSGAE VKKPGASVKV SCKASGYTFT SSWMHWARQA |
| | Variable | PGQGLEWIGE |
| | | IHPNSGGTNY AQKFQGRVTM TVDTSISTAY MELSRLRSDD |
| | | TAVYYCARGD |
| | | YYGYVSWFAY WGQGTLVTVS S |
| 293 | HC | QVQLVQSGAE VKKPGASVKV SCKASGYTFT SSWMHWARQA |
| | Variable | PGQGLEWMGE |
| | | IHPNSGGTNY AQKFQGRVTM TVDTSISTAY MELSRLRSDD |
| | | TAVYYCARGD |
| | | YYGYVSWFAY WGQGTLVTVS S |
| 294 | LC | DIQMNQSPSS LSASLGDTIT ITCHASQNIN VLLSWYQQKP |
| | Variable | GNIPKLLIYK |
| | | ASNLHTGVPS RFSGSGSGTG FTFTISSLOP EDIATYYCQQ |
| | | GQSYPYTFGG |
| | | GTKLEIK |
| 295 | LC | DIQMTQSPSS LSASVGDRVT ITCQASQDIS NYLNWYQQKP |
| | Variable | GKAPKLLIYD |
| | | ASNLETGVPS RFSGSGSGTD FTFTISSLOP EDIATYYCQQ |
| | | YDNLPYTFGQ |
| | | GTKLEIK |
| 296 | LC | DIQMTQSPSS LSASVGDRVT ITCQASQNIN VLLNWYQQKP |
| | Variable | GKAPKLLIYK |
| | | ASNLHTGVPS RFSGSGSGTD FTFTISSLOP EDIATYYCQQ |
| | | GQSYPYTFGQ |
| | | GTKLEIK |
| 297 | LC | DIQMNQSPSS LSASVGDRVT ITCQASQNIN VLLSWYQQKP |
| | Variable | GKAPKLLIYK |
| | | ASNLHTGVPS RFSGSGSGTD FTFTISSLOP EDIATYYCQQ |
| | | GQSYPYTFGQ |
| | | GTKLEIK |
| 298 | HCDR1 | GYTFTSYDIN |
| 299 | HCDR2 | WLNPNSGXTG; X = N, Y |
| 300 | HCDR3 | EVPETAAFEY |
| 301 | LCDR1 | TSSSSDIGA(X1)(X2)GV(X3); X1 = G, A; X2 = L, S, Q; X3 = H, L |
| 302 | LCDR2 | GYYNRPS |
| 303 | LCDR3 | QSXDGTLSAL; X = Y, W, F |
| 304 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 305 | LC | QSVLTQPPSV SGAPGQRVTI SCTSSSSDIG AXXGVXWYQQ |
| | Variable | LPGTAPKLLI |
| | | EGYYNRPSGV PDRFSGSKSG TSASLTITGL LPEDEGDYYC |
| | | QSXDGTLSAL |
| | | FGGGTKLTVL G |
| 306 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 307 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 308 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 309 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSYDGTLSAL |
| | | FGGGTKLTVLG |
| 310 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 311 | LC | QSVLTQPPSV SGAPGQRVTI SCTSSSSDIG AALGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 312 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 313 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 314 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 315 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 316 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 317 7 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 318 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 319 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVLG |
| 320 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 321 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 322 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 323 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 324 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 325 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 326 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 327 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSFDGTLSAL |
| | | FGGGTKLTVLG |
| 328 | HCDR1 | SYFWS |
| 329 | HCDR2 | YIYYSGNTKYNPSLKS |
| 330 | HCDR3 | ETGSYYGFDY |
| 331 | LCDR1 | RASQSINNYLN |
| 332 | LCDR2 | AASSLOS |
| 333 | LCDR3 | QQSYSTPRT |
| 334 | HC | QVQLQESGPGLVKPSETLSLTCTVSGGSISSYFWSWIRQPPGKGLEWIGY |
| | Variable | IYYSGNTKYNPSLKSRVTISIDTSKNQFSLKLSSVTAADTAVYYCARETG |
| | | SYYGFDYWGQGTLVTVSS |
| 335 | LC | DIQMTQSPSSLSASVGDRVTITCRASQSINNYLNWYQQRPGKAPKLLIY |
| | Variable | AASSLQSGVPSRFSGSGSGTDFTLTISSLQPGDFATYYCQQSYSTPRTFG |
| | | QGTKLEIK |
| 336 | HCDR1 | GYYWN |
| 337 | HCDR2 | EINHAGNTNYNPSLKS |
| 338 | HCDR3 | GYCRSTTCYFDY |
| 339 | LCDR1 | RASQSVRSSYLA |
| 340 | LCDR2 | GASSRAT |
| 341 | LCDR3 | QQYGSSPT |
| 342 | HC | QVQLQQWGAGLLKPSETLSLTCAVHGGSFSGYYWNWIRQPPGKGLEW |
| | Variable | IGEINHAGNTNYNPSLKSRVTISLDTSKNQFSLTLTSVTAADTAVYYCAR |
| | | GYCRSTTCYFDYWGQGTLVTVSS |
| 343 | LC | EIVLTQSPGTLSLSPGERATLSCRASQSVRSSYLAWYQQKPGQAPRLLIY |
| | Variable | GASSRATGIPDRFSGSGSGTDFTLTISRLEPEDFAVYYCQQYGSSPTFGQ |
| | | GTRLEIK |
| 344 | HC | EVQLQQSGAELVKPGASVKLSCTASGFDIQDTYMHWVKQRPEQGLEWI |
| | Variable | GRIDPASGHTKYDPKFQVKATITTDTSSNTAYLQLSSLTSEDTAVYYCS |
| | | RSGGLPDVWGAGTTVTVSS |
| 345 | LC | QIVLSQSPAILSASPGEKVTMTCRASSSVSYMYWYQQKPGSSPKPWIYA |
| | Variable | TSNLASGVPDRFSGSGSGTSYSLTISRVEAEDAATYYCQQWSGNPRTFG |
| | | GGTKLEIK |
| 346 | HCDR1 | GFDIQDTYMH |
| 347 | HCDR2 | RIDPASGHTKYDPKFQV |
| 348 | HCDR3 | SGGLPDV |
| 349 | LCDRI | RASSSVSYMY |
| 350 | LCDR2 | ATSNLAS |
| 351 | LCDR3 | QQWEGNPRT |
| 352 | HC | QVQLVQSGAEVKKPGASVKLSCKASGFDIQDTYMHWVRQAPGQGLE |
| | Variable | WMGRIDPASGHTKYDPKFQVRVTMTTDTSTSTVYMELSSLRSEDTAVY |
| | | YCSRSGGLPDVWGQGTTVTVSS |
| 353 | LC | EIVLTQSPGTLSLSPGERVTMSCRASSSVSYMYWYQQKPGQAPRPWIYA |
| | Variable | TSNLASGVPDRFSGSGSGTDYTLTISRLEPEDFAVYYCQQWSGNPRTFG |
| | | GGTKLEIK |
| 354 | (CDR- | QVQLVQSGAEVKKPGASVKLSCKASGFDIQDTYMHWVRQAPGQGLE |
| | grafted | WMGRIDPASGHTKYDPKFQVRVTMTRDTSTSTVYMELSSLRSEDTAVY |
| | LC) HC | YCSRSGGLPDVWGQGTTVTVSS |
| | variable | |
| | region | |
| 355 | (CDR- | EIVLTQSPGTLSLSPGERATLSCRASSSVSYMYWYQQKPGQAPRLLIYA |
| | grafted | TSNLASGIPDRFSGSGSGTDFTLTISRLEPEDFAVYYCQQWSGNPRTFGG |
| | LC) HC | GTKLEIK |
| | variable | |
| | region | |
| 356 | (CDR- | QVQLVQSGAEVKKPGASVKVSCKASGFDIQDTYMHWVRQAPGQGLE |
| | grafted | WMGRIDPASGHTKYDPKFQVRVTMTRDTSTSTVYMELSSLRSEDTAVY |
| | HC) HC | YCARSGGLPDVWGQGTTVTVSS |
| | variable | |
| | region | |
| 357 | (CDR- | EIVLTQSPGTLSLSPGERATLSCRASSSVSYMYWYQQKPGQAPRLLIYA |
| | grafted | TSNLASGVPDRFSGSGSGTDYTLTISRLEPEDFAVYYCQQWSGNPRTFG |
| | HC) LC | GGTKLEIK |
| | variable | |
| | region | |
| 358 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYNCA |
| | | RRKDGNYYYAMDYWGQGTSVTVSS |
| 359 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYYCA |
| | | RRKDGNYYYAMDYWGQGTSVTVSS |
| 360 | HC | EVQLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYNC |
| | | ARRKDGNYYYAMDYWGQGTTVTVSS |
| 361 | HC | EVOLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYYC |
| | | ARRKDGNYYYAMDYWGQGTTVTVSS |
| 362 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYNC |
| | | ARRKDGNYYYAMDYWGQGTTVTVSS |
| 363 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYYC |
| | | ARRKDGNYYYAMDYWGQGTTVTVSS |
| 364 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYNCA |
| | | RRKDGNYYYAMDYWGQGTTVTVSS |
| 365 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYYCA |
| | | RRKDGNYYYAMDYWGQGTTVTVSS |
| 366 | HC | QVQLVQSGSELKKPGASVKVSCKASGFTFTNYAMSWVRQAPGKRLEW |
| | variable | VSTITSGGSYIYYLDSVKGRFTISRENAKSTLYLQMNSLRTEDTALYNCA |
| | | RRKDGNYYYAMDYWGQGTTVTVSS |
| 367 | HC | QVQLVQSGSELKKPGASVKVSCKASGFTFTNYAMSWVRQAPGKRLEW |
| | variable | VATITSGGSYIYYLDSVKGRFTISRENAKSTLYLQMNSLRTEDTALYYC |
| | | ARRKDGNYYYAMDYWGQGTTVTVSS |
| 368 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAVYNC |
| | | ARRKDGNYYYAMDYWGQGTTVTVSS |
| 369 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAIYYC |
| | | ARRKDGNYYYAMDYWGQGTTVTVSS |
| 370 | HC | EVMLLQSGAEVKKPGASVKVSCKASGFTFTNYAMSWVRQAPGQRLE |
| | variable | WVATITSGGSYIYYLDSVKGRFTISRDNAKSTLHLQMNSLRAEDTAVY |
| | | YCARRKDGNYYYAMDYWGQGTTVTVSS |
| 371 | LC | DIVLTQSPASLAVSLGQRATISCRASESVDSYGNSFIHWYQQKAGQPPK |
| | variable | LLIYRASNLESGIPARFSGSGSRTDFTLTINPVEADDVATYYCQQSYEDP |
| | | WTFGGGTKLEIK |
| 372 | LC | DIVLTQSPATLSLSPGERATLSCRASESVDSYGNSFIHWYQQKPGQPPKL |
| | variable | LIYRASNLESGIPARFSGSGSRTDFTLTISSLEPEDFAVYYCQQSYEDPWT |
| | | FGGGTKXEIK |
| 373 | LC | DIVLTQSPSSLSASVGDRVTITCRASESVDSYGNSFIHWYQQKPGQPPKL |
| | variable | LIYRASNLESGIPARFSGSGSRTDFTLTISSLQPEDFATYYCQQSYEDPWT |
| | | FGGGTKXEIK |
| 374 | LC | DIVLTQSPDFQSVTPKEKVTITCRASESVDSYGNSFIHWYQQKPGQPPKL |
| | variable | LIYRASNLESGIPARFSGSGSRTDFTLTISSLEAEDAATYYCQQSYEDPW |
| | | TFGGGTKXEIK |
| 375 | LC | DIVLTQTPLSLSVTPGQPASISCRASESVDSYGNSFIHWYQQKPGQPPKL |
| | variable | LIYRASNLESGIPARFSGSGSRTDFTLKISRVEAEDVGVYYCQQSYEDPW |
| | | TFGGGTKXEIK |
| 376 | HCDR1 | TYGMS |
| 377 | HCDR2 | WMNTYSGVTTYADDFKG |
| 378 | HCDR3 | EGYVFDDYYATDY |
| 379 | LCDR1 | RSSQNIVHSDGNTYLE |
| 380 | LCDR2 | KVSNRFS |
| 381 | LCDR3 | FQGSHVPLT |
| 382 | HC | QIQLVQSGPELKKPGETVKISCKASGYTFTTYGMSWVKQAPGKGLKW |
| | Variable | MGWMNTYSGVTTYADDFKGRFAFSLETSASTAYMQIDNLKNEDTATY |
| | | FCAREGYVFDDYYATDYWGQGTSVTVSS |
| 383 | LC | DVLMTQTPLSLPVSLGDQASISCRSSQNIVHSDGNTYLEWYLQKPGQSP |
| | Variable | KLLIYKVSNRFSGVPDRFSGSGSGTDFTLKISRVEAEDLGIYYCFQGSHV |
| | | PLTFGAGTKLELK |
| 384 | HCDR1 | KYDIN |
| 385 | HCDR2 | WIFPGDGRTDYNEKFKG |
| 386 | HCDR3 | YGPAMDY |
| 387 | LCDR1 | RSSQTIVHSNGDTYLD |
| 388 | LCDR2 | KVSNRFS |
| 389 | LCDR3 | FQGSHVPYT |
| 390 | HC | MGWSWVFLFLLSVTAGVHSQVHLQQSGPELVKPGASVKLSCKASGYT |
| | Variable | FTKYDINWVRQRPEQGLEWIGWIFPGDGRTDYNEKFKGKATLTTDKSS |
| | | STAYMEVSRLTSEDSAVYFCARYGPAMDYWGQGTSVTVAS |
| 391 | LC | MKLPVRLLVLMFWIPASSSDVLMTQTPLSLPVSLGDQASISCRSSQTIVH |
| | Variable | SNGDTYLDWFLQKPGQSPKLLIYKVSNRFSGVPDRFSGSGSGTDFTLKIS |
| | | RVEAEDLGVYYCFQGSHVPYTFGGGTKLEIK |

TABLE 19
Non-Limiting Examples of anti-TL1A and anti-DR3 Antibodies

| | HC Variable Domain | LC Variable Domain |
|---|---|---|
| Antibody Name | (SEQ ID NO) | (SEQ ID NO) |
| A100 | 215 | 216 |
| A101 | 223 | 224 |
| A102 | 231 | 232 |
| A103 | 242 | 243 |
| A104 | 252 | 253 |
| A105 | 260 | 261 |
| A106 | 271 | 275 |
| A107 | 271 | 276 |
| A108 | 271 | 277 |
| A109 | 271 | 278 |
| A110 | 271 | 279 |
| A111 | 271 | 280 |
| A112 | 271 | 281 |
| A113 | 271 | 282 |
| A114 | 272 | 275 |
| A115 | 272 | 276 |
| A116 | 272 | 277 |
| A117 | 272 | 278 |
| A118 | 272 | 279 |
| A119 | 272 | 280 |
| A120 | 272 | 281 |
| A121 | 272 | 282 |
| A122 | 273 | 275 |
| A123 | 273 | 276 |
| A124 | 273 | 277 |

TABLE 20
Non-Limiting Examples of Kinase Modulator

| (A) Kinase Target | (B) Kinase Modulator |
|---|---|
| PDK1 (pyruvate | Celecoxib, 7-Hydroxystaurosporine, Bisindolylmaleimide VIII, Staurosporine, |
| dehydrogenase | Dexfosfoserine, 10,11-dimethoxy-4-methyldibenzo[c,f]-2,7-naphthyridine-3,6- |
| kinase 1) | diamine; 5-hydroxy-3-[(1r)-1-(1h-pyrrol-2-yl)ethyl]-2h-indol-2-one; 1-{2-oxo-3- |
| | [(1r)-1-(1h-pyrrol-2-yl)ethyl]-2h-indol-5-yl}urea; 2-(1H-imidazol-1-yl)-9- |
| | methoxy-8-(2-methoxyethoxy)benzo[c][2,7]naphthyridin-4-amine; |
| | Bisindolylmaleimide I; 3-(1H-indol-3-yl)-4-(1-{2-[(2S)-1- |
| | methylpyrrolidinyl]ethyl}-1H-indol-3-yl)-1H-pyrrole-2,5-dione; 3-[1-(3- |
| | aminopropyl)-1h-indol-3-yl]-4-(1h-indol-3-yl)-1h-pyrrole-2,5-dione; Inositol |
| | 1,3,4,5-Tetrakisphosphate; Fostamatinib; AR-12 (Arno Therapeutics) |
| CDK11B (cyclin- | Phosphonothreonine, Alvocidib, SNS-032, Seliciclib |
| dependent kinase | |
| 11B) | |
| ULK1 | Fostamatinib |
| (Serine/threonine- | |
| protein kinase | |
| ULK1) | |
| RIPK 1 (receptor- | Fostamatinib |
| interacting | |
| serine/threonine- | |
| protein kinase 1) | |
| IKBKB (inhibitor | Auranofin, Arsenic trioxide, MLN0415, Ertiprotafib, Sulfasalazine, Mesalazine, |
| of nuclear factor | Acetylcysteine, Fostamatinib, Acetylsalicylic acid |
| kappa-B kinase | |
| subunit beta) | |
| CDK9 (cyclin- | Riviciclib, Roniciclib, Seliciclib, Alvocidib, ATUVECICLIB, SNS-032 (BMS- |
| dependent kinase | 387032), AZD-5438 (AstraZeneca) |
| 9) | |
| STK11 | Metformin, magnesium, manganese, cyclic AMP, ATP, Midostaurin, Nintedanib, |
| (serine/threonine | Ruboxistaurin, Sunitinib, ADP |
| kinase 11) | |
| RAF1 (RAF proto- | Balamapimod, Dabrafenib, Regorafenib, Sorafenib, LErafAON, iCo-007, |
| oncogene | XL281, Cholecystokinin, Fostamatinib |
| serine/threonine- | |
| protein kinase) | |
| CSNK1A1 (Casein | Fostamatinib, IC261, ATP, PF 670462, CKI 7 dihydrochloride, ADP, (R)- |
| Kinase 1 Alpha 1) | DRF053 dihydrochloride, D4476, LH846, PF 4800567 hydrochloride, PF |
| | 670462, CKI 7 dihydrochloride, IC261, Ruxolitinib, Bosutinib, Sorafenib, A14, |
| | A64, A47, A75, A51, A86 Sunitinib |
| AURKB (Aurora | Barasertib, Cenisertib, Danusertib, Ilorasertib, Tozasertib, Hesperidin, AT9283, |
| kinase B) | Enzastaurin, Reversine, Fostamatinib |
| ATR | Ceralasertib, Berzosertib, diphenyl acetamidotrichloroethyl fluoronitrophenyl |
| (serine/threonine- | thiourea, BAY-1895344, Nevanimibe hydrochloride |
| protein kinase | |
| ATR) | |
| PRKAA2 (5′-AMP- | Acetylsalicylic acid, Fostamatinib, Topiramate, Adenosine phosphate |
| activated protein | |
| kinase catalytic | |
| subunit alpha-2) | |
| CHEK2 | Prexasertib |
| (checkpoint kinase | |
| 2) | |
| PRKDC (DNA- | Wortmannin, Torin 2, PIK-75, peposertib, KU-0060648, AZD7648, NU-7441, |
| dependent protein | PI-103, PP121, DNA-PK inhibitor III, NU-7026, DNA-PK inhibitor V, |
| kinase catalytic | Trifluoperazine, Suramin, Idelalisib |
| subunit) | |
| AURKA (Aurora | Alisertib, Cenisertib, Tozasertib, Danusertib, Ilorasertib, Phosphonothreonine, |
| Kinase A) | CYC116, AT9283, SNS-314, MLN8054, Enzastaurin, 4-(4-methylpiperazin-1- |
| | yl)-n-[5-(2-thienylacetyl)-1,5-dihydropyrrolo[3,4-c]pyrazol-3-yl]benzamide, |
| | AKI-001, 1-{5-[2-(thieno[3,2-d]pyrimidin-4-ylamino)ethyl]-1,3-thiazol-2-yl}-3- |
| | [3-(trifluoromethyl)phenyl]urea; 1-(5-{2-[(1-methyl-1H-pyrazolo[4,3- |
| | d]pyrimidin-7-yl)amino]ethyl}-1,3-thiazol-2-yl)-3-[3- |
| | (trifluoromethyl)phenyl]urea; N-{3-[(4-{[3- |
| | (trifluoromethyl)phenyl]amino}pyrimidin-2- |
| | yl)amino]phenyl}cyclopropanecarboxamide; N-butyl-3-{[6-(9H-purin-6- |
| | ylamino)hexanoyl]amino}benzamide; Fostamatinib |
| RPS6KB1 | LY2584702, PF-4708671, GNE-3511 |
| (Ribosomal Protein | |
| S6 Kinase B1) | |
| CSNK2A2 (Casein | Silmitasertib, [1-(6-{6-[(1-methylethyl)amino]-1H-indazol-1-yl}pyrazin-2-yl)- |
| kinase II subunit | 1H-pyrrol-3-yl]acetic acid, Fostamatinib |
| alpha) | |
| PLK1 | Rigosertib, Volasertib, 3-[3-chloro-5-(5-{[(1S)-1- |
| (Serine/threonine- | phenylethyl]amino}isoxazolo[5,4-c]pyridin-3-yl)phenyl]propan-1-ol; 3-[3-(3- |
| protein kinase | methyl-6-{[(1S)-1-phenylethyl]amino}-1H-pyrazolo[4,3-c]pyridin-1- |
| PLK1) | yl)phenyl]propenamide; 4-(4-methylpiperazin-1-yl)-n-[5-(2-thienylacetyl)-1,5- |
| | dihydropyrrolo[3,4-c]pyrazol-3-yl]benzamide; 1-[5-Methyl-2- |
| | (trifluoromethyl)furan-3-yl]-3-[5-[2-[[6-(1H-1,2,4-triazol-5-ylamino)pyrimidin- |
| | 4-yl]amino]ethyl]-1,3-thiazol-2-yl]urea; Wortmannin, Fostamatinib, |
| | Onvansertib, HMN-214, Purpurogallin, BI-2536, GSK-461364, Tak-960, |
| | Volasertib trihydrochloride, Rigosertib sodium, BI-2536 monohydrate |
| PRKAA1 (5′-AMP- | Adenosine phosphate, ATP, Phenformin, Fostamatinib |
| activated protein | |
| kinase catalytic | |
| subunit alpha-1) | |
| MTOR | Vistusertib, Sapanisertib, Bimiralisib, Samotolisib, Panulisib, Omipalisib, |
| (Serine/threonine- | Apitolisib, Voxtalisib, Dactolisib, Gedatolisib, SF1126, Rimiducid, XL765, |
| protein kinase | Everolimus, Ridaforolimus, Temsirolimus, Sirolimus, Pimecrolimus, |
| mTOR) | Fostamatinib, PKI-179, PF-04691502, GDC-0349, GSK-1059615, AZD-8055, |
| | CC-115, BGT-226, Sonolisib, MKC-1, Umirolimus, VS-5584, Onatasertib, |
| | Paxalisib, Bimiralisib, 2-Hydyroxyoleic acid, Ophiopogonin B, GNE-493, GNE- |
| | 477, Guttiferone E, PF-04979064, Hypaphorine, Astragaloside II, PP-121, KU- |
| | 0063794, PD-166866, PI-103, CGP-60474, AZD-1208, PP-242, AZD-1897, LY- |
| | 294002, SF-1126, Licochalcone A, Puquitinib, Zotarolimus, Ridaforolimus, |
| | Tacrolimus, Voxtalisib hydrochloride, Bimiralisib hydrochloride, Bimiralisib |
| | hydrochloride monohydrate, Dactolisib tosylate, Hypaphorine hydrochloride |
| CDK1 (cyclin- | Roniciclib, Riviciclib, Milciclib, Alsterpaullone, Alvocidib, Hymenialdisine, |
| dependent kinase | Indirubin-3′-monoxime, Olomoucine, SU9516, AT-7519, Seliciclib, |
| 1) | Fostamatinib, OTX-008, K-00546 |
| CDK2 (cyclin- | Bosutinib, Roniciclib, Seliciclib, 4-[5-(Trans-4-Aminocyclohexylamino)-3- |
| dependent kinase | Isopropylpyrazolo[1,5-a]Pyrimidin-7-Ylamino]-N,N- |
| 2) | Dimethylbenzenesulfonamide; Staurosporine; 4-(2,4-Dimethyl-Thiazol-5-Yl)- |
| | Pyrimidin-2-Ylamine; Olomoucine; 4-[(4-Imidazo[1,2-a]Pyridin-3-Ylpyrimidin- |
| | 2-Yl)Amino]Benzenesulfonamide; 2-Amino-6-Chloropyrazine; 6-O- |
| | Cyclohexylmethyl Guanine; N-[4-(2-Methylimidazo[1,2-a]Pyridin-3-Yl)-2- |
| | Pyrimidinyl]Acetamide; 1-Amino-6-Cyclohex-3-Enylmethyloxypurine; N-(5- |
| | Cyclopropyl-1h-Pyrazol-3-Yl)Benzamide; Purvalanol; [4-(2-Amino-4-Methyl- |
| | Thiazol-5-Yl)-Pyrimidin-2-Yl]-(3-Nitro-Phenyl)-Amine; (5R)-5-{[(2-Amino-3H- |
| | purin-6-yl)oxy]methyl}-2-pyrrolidinone; 4-(2,4-Dimethyl-1,3-thiazol-5-yl)-N-[4- |
| | (trifluoromethyl)phenyl]-2-pyrimidinamine; Hymenialdisine; (5- |
| | Chloropyrazolo[1,5-a]Pyrimidin-7-Yl)-(4-Methanesulfonylphenyl)Amine; 4-(5- |
| | Bromo-2-Oxo-2h-Indol-3-Ylazo)-Benzenesulfonamide; 4-(2,5-Dichloro- |
| | Thiophen-3-Yl)-Pyrimidin-2-Ylamine; 4-[(6-Amino-4- |
| | Pyrimidinyl)Amino]Benzenesulfonamide; 4-[3-Hydroxyanilino]-6,7- |
| | Dimethoxyquinazoline; SU9516; 3-Pyridin-4-Yl-2,4-Dihydro-Indeno[1,2- |
| | .C.]Pyrazole; (2E,3S)-3-hydroxy-5′-[(4-hydroxypiperidin-1-yl)sulfonyl]-3- |
| | methyl-1,3-dihydro-2,3′-biindol-2′(1′H)-one; 1-[(2-Amino-6,9-Dihydro-1h-Purin- |
| | 6-Yl)Oxy]-3-Methyl-2-Butanol; 4-((3r,4s,5r)-4-Amino-3,5-Dihydroxy-Hex-1- |
| | Ynyl)-5-Fluoro-3-[1-(3-Methoxy-1h-Pyrrol-2-Yl)-Meth-(Z)-Ylidene]-1,3- |
| | Dihydro-Indol-2-One; Lysine Nz-Carboxylic Acid; [2-Amino-6-(2,6-Difluoro- |
| | Benzoyl)-Imidazo[1,2-a]Pyridin-3-Yl]-Phenyl-Methanone; N′-[4-(2,4-Dimethyl- |
| | 1,3-thiazol-5-yl)-2-pyrimidinyl]-N-hydroxyimidoformamide; N′- |
| | (Pyrrolidino[2,1-B]Isoindolin-4-On-8-Yl)-N-(Pyridin-2-Yl)Urea; 2-[Trans-(4- |
| | Aminocyclohexyl)Amino]-6-(Benzyl-Amino)-9-Cyclopentylpurine; 4-[4-(4- |
| | Methyl-2-Methylamino-Thiazol-5-Yl)-Pyrimidin-2-Ylamino]-Phenol 3-[4- |
| | (2,4-Dimethyl-Thiazol-5-Yl)-Pyrimidin-2-Ylamino]-Phenol; |
| | phenylaminoimidazo(1,2-alpha)pyridine; Olomoucine II; Triazolopyrimidine; |
| | Alvocidib; Seliciclib; 4-[(7-oxo-7h-thiazolo[5,4-e]indol-8-ylmethyl)-amino]-n- |
| | pyridin-2-yl-benzenesulfonamide; (13R,15S)-13-methyl-16-oxa-8,9,12,22,24- |
| | pentaazahexacyclo[15.6.2.16,9.1,12,15.0,2,7.0,21,25]heptacosa- |
| | 1(24),2,4,6,17(25),18,20-heptaene-23,26-dione; N-(3-cyclopropyl-1H-pyrazol-5- |
| | yl)-2-(2-naphthyl)acetamide; 2-anilino-6-cyclohexylmethoxypurine; 1-(5-OXO- |
| | 2,3,5,9B-tetrahydro-1h-pyrrolo[2,1-a]isoindol-9-yl)-3-(5-pyrrolidin-2-yl-1h- |
| | pyrazol-3-yl)-urea; (5-phenyl-7-(pyridin-3-ylmethylamino)pyrazolo[1,5- |
| | a]pyrimidin-3-yl)methanol; 2-(3,4-dihydroxyphenyl)-8-(1,1- |
| | dioxidoisothiazolidin-2-yl)-3-hydroxy-6-methyl-4h-chromen-4-one; (2R)-1- |
| | (dimethylamino)-3-{4-[(6-{[2-fluoro-5- |
| | (trifluoromethyl)phenyl]amino}pyrimidin-4-yl)amino]phenoxy}propan-2-ol; 5- |
| | (2,3-dichlorophenyl)-N-(pyridin-4-ylmethyl)-3-thiocyanatopyrazolo[1,5- |
| | a]pyrimidin-7-amine; O6-cyclohexylmethoxy-2-(4’-sulphamoylanilino) purine; |
| | (2S)-N-[(3E)-5-Cyclopropyl-3H-pyrazol-3-ylidene]-2-[4-(2-oxo-]- |
| | imidazolidinyl)phenyl]propenamide; 5-[(2-aminoethyl)amino]-6-fluoro-3-(1h- |
| | pyrrol-2-yl)benzo[cd]indol-2(1h)-one; N-cyclopropyl-4-pyrazolo[1,5- |
| | b]pyridazin-3-ylpyrimidin-2-amine; 3-((3-bromo-5-o-tolylpyrazolo[1,5- |
| | a]pyrimidin-7-ylamino)methyl)pyridine 1-oxide; 6-cyclohexylmethoxy-2-(3′- |
| | chloroanilino) purine; 3-bromo-5-phenyl-N-(pyridin-4-ylmethyl)pyrazolo[1,5- |
| | a]pyrimidin-7-amine; N-[5-(1,1-dioxidoisothiazolidin-2-yl)-1h-indazol-3-yl]-2- |
| | (4-piperidin-1-ylphenyl)acetamide; (3R)-3-(aminomethyl)-9-methoxy-1,2,3,4- |
| | tetrahydro-5H-[1]benzothieno[3,2-e][1,4]diazepin-5-one; 5-[5,6-bis(methyloxy)- |
| | 1h-benzimidazol-1-yl]-3-{[1-(2-chlorophenyl)ethyl]oxy}-2- |
| | thiophenecarboxamide; 5-Bromoindirubin; (2S)-1-{4-[(4-Anilino-5-bromo-2- |
| | pyrimidinyl)amino]phenoxy}-3-(dimethylamino)-2-propanol; (2R)-1-{4-[(4- |
| | Anilino-5-bromo-2-pyrimidinyl)amino]phenoxy}-3-(dimethylamino)-2- |
| | propanol; (5E)-2-Amino-5-(2-pyridinylmethylene)-1,3-thiazol-4(5H)-one; 4-{5- |
| | [(Z)-(2,4-dioxo-1,3-thiazolidin-5-ylidene)methyl]furan-2- |
| | yl}benzenesulfonamide; 4-{5-[(Z)-(2-imino-4-oxo-1,3-thiazolidin-5- |
| | ylidene)methyl]-2-furyl}-n-methylbenzenesulfonamide; 4-{5-[(Z)-(2-imino-4- |
| | oxo-1,3-thiazolidin-5-ylidene)methyl]furan-2-yl}benzenesulfonamide; 4-{5-[(Z)- |
| | (2-imino-4-oxo-1,3-thiazolidin-5-ylidene)methyl]furan-2-yl}-2- |
| | (trifluoromethyl)benzenesulfonamide; 4-{5-[(Z)-(2-imino-4-oxo-1,3-thiazolidin- |
| | 5-ylidene)methyl]furan-2-yl}benzoic acid; 4-{5-[(1Z)-1-(2-imino-4-oxo-1,3- |
| | thiazolidin-5-ylidene)ethyl]-2-furyl}benzenesulfonamide; N-[4-(2,4-dimethyl- |
| | thiazol-5-yl)-pyrimidin-2-yl]-n′,n′-dimethyl-benzene-1,4-diamine; (5Z)-5-(3- |
| | bromocyclohexa-2,5-dien-1-ylidene)-n-(pyridin-4-ylmethyl)-1,5- |
| | dihydropyrazolo[1,5-a]pyrimidin-7-amine; 6-(3,4-dihydroxybenzyl)-3-ethyl-1- |
| | (2,4,6-trichlorophenyl)-1h-pyrazolo[3,4-d]pyrimidin-4(5h)-one; 6-(3- |
| | aminophenyl)-n-(tert-butyl)-2-(trifluoromethyl)quinazolin-4-amine; 2-(4- |
| | (aminomethyl)piperidin-1-yl)-n-(3_cyclohexyl-4-oxo-2,4-dihydroindeno[1,2- |
| | c]pyrazol-5-yl)acetamide; 1-(3-(2,4-dimethylthiazol-5-yl)-4-oxo-2,4- |
| | dihydroindeno[1,2-c]pyrazol-5-yl)-3-(4-methylpiperazin-1-yl)urea; 4-{[5- |
| | (cyclohexylmethoxy)[1,2,4]triazolo[1,5-a]pyrimidin-7- |
| | yl]amino}benzenesulfonamide; 4-{[5-(cyclohexylamino)[1,2,4]triazolo[1,5- |
| | a]pyrimidin-7-yl]amino}benzenesulfonamide; 4-({5-[(4- |
| | aminocyclohexyl)amino][1,2,4]triazolo[1,5-a]pyrimidin-7- |
| | yl}amino)benzenesulfonamide; 4-{[5-(cyclohexyloxy)[1,2,4]triazolo[1,5- |
| | a]pyrimidin-7-yl]amino}benzenesulfonamide; CAN-508; (2R)-1-[4-({4-[(2,5- |
| | Dichlorophenyl)amino]-2-pyrimidinyl}amino)phenoxy]-3-(dimethylamino)-2- |
| | propanol; (2S)-1-[4-({6-[(2,6-Difluorophenyl)amino]-4- |
| | pyrimidinyl}amino)phenoxy]-3-(dimethylamino)-2-propanol; (2S)-1-[4-({4- |
| | [(2,5-Dichlorophenyl)amino]-2-pyrimidinyl}amino)phenoxy]-3- |
| | (dimethylamino)-2-propanol; (2R)-1-[4-({6-[(2,6-Difluorophenyl)amino]-4- |
| | pyrimidinyl}amino)phenoxy]-3-(dimethylamino)-2-propanol; N-(2- |
| | methoxyethyl)-4-({4-[2-methyl-1-(1-methylethyl)-1h-imidazol-5-yl]pyrimidin-2- |
| | yl}amino)benzenesulfonamide; 4-{[4-(1-cyclopropyl-2-methyl-1h-imidazol-5- |
| | yl)pyrimidin-2-yl]amino}-n-methylbenzenesulfonamide; 1-(3,5-dichlorophenyl)- |
| | 5-methyl-1h-1,2,4-triazole-3-carboxylic acid; (2S)-1-(Dimethylamino)-3-(4-{[4- |
| | (2-methylimidazo[1,2-a]pyridin-3-yl)-2-pyrimidinyl]amino}phenoxy)-2- |
| | propanol; N-(4-{[(3S)-3-(dimethylamino)pyrrolidin-1-yl]carbonyl}phenyl)-5- |
| | fluoro-4-[2-methyl-1-(1-methylethyl)-1H-imidazol-5-yl]pyrimidin-2-amine; 2- |
| | {4-[4-({4-[2-methyl-1-(1-methylethyl)-1H-imidazol-5-yl]pyrimidin-2- |
| | yl}amino)phenyl]piperazin-1-yl}-2-oxoethanol; Indirubin-3′-monoxime; N-[3- |
| | (1H-benzimidazol-2-yl)-1h-pyrazol-4-yl]benzamide; RO-4584820; N-Methyl-4- |
| | {[(2-oxo-1,2-dihydro-3H-indol-3-ylidene)methyl]amino}benzenesulfonamide; |
| | N-methyl-{4-[2-(7-oxo-6,7-dihydro-8H-[1,3]thiazolo[5,4-e]indol-8- |
| | ylidene)hydrazino]phenyl}methanesulfonamide; 3-{[(2,2-dioxido-1,3-dihydro-2- |
| | benzothien-5-yl)amino]methylene}-5-(1,3-oxazol-5-yl)-1,3-dihydro-2H-indol-2- |
| | one; 4-{[(2-Oxo-1,2-dihydro-3H-indol-3-ylidene)methyl]amino}-N-(1,3-thiazol- |
| | 2-yl)benzenesulfonamide; 3-{[4- |
| | ([amino(imino)methyl]aminosulfonyl)anilino]methylene}-2-oxo-2,3-dihydro- |
| | 1H-indole; 5-hydroxynaphthalene-1-sulfonamide; N-(4-sulfamoylphenyl)-1H- |
| | indazole-3-carboxamide4-[(6-chloropyrazin-2-yl)amino]benzenesulfonamide; N- |
| | phenyl-1H-pyrazole-3-carboxamide; 4-(acetylamino)-N-(4-fluorophenyl)-1H- |
| | pyrazole-3-carboxamide; (4E)-N-(4-fluorophenyl)-4-[(phenylcarbonyl)imino]- |
| | 4H-pyrazole-3-carboxamide; {[(2,6-difluorophenyl)carbonyl]amino}-N-(4- |
| | fluorophenyl)-1H-pyrazole-3-carboxamide; 5-chloro-7-[(1- |
| | methylethyl)amino]pyrazolo[1,5-a]pyrimidine-3-carbonitrile; 5-[(4- |
| | aminocyclohexyl)amino]-7-(propan-2-ylamino)pyrazolo[1,5-a]pyrimidine-3- |
| | carbonitrile; 4-{[(2,6-difluorophenyl)carbonyl]amino}-N-[(3S)-piperidin-3-yl]- |
| | 1H-pyrazole-3-carboxamide; AT-7519; 4-(4-methoxy-1H-pyrrolo[2,3-b]pyridin- |
| | 3-yl)pyrimidin-2-amine; 4-(4-propoxy-1H-pyrrolo[2,3-b]pyridin-3-yl)pyrimidin- |
| | 2-amine; hydroxy(oxo)(3-{[(2z)-4-[3-(1h-1,2,4-triazol-1- |
| | ylmethyl)phenyl]pyrimidin-2(5h)-ylidene]amino}phenyl)ammonium; 4-Methyl- |
| | 5-[(2Z)-2-{[4-(4-morpholinyl)phenyl]imino}-2,5-dihydro-4-pyrimidinyl]-1,3- |
| | thiazol-2-amine; 6-cyclohexylmethyloxy-2-(4′-hydroxyanilino)purine; 4-(6- |
| | cyclohexylmethoxy-9h-purin-2-ylamino)-benzamide; 6-(cyclohexylmethoxy)- |
| | 8-isopropyl-9h-purin-2-amine; 3-(6-cyclohexylmethoxy-9h-purin-2-ylamino)- |
| | benzenesulfonamide; (2R)-2-{[4-(benzylamino)-8-(1-methylethyl)pyrazolo[1,5- |
| | a][1,3,5]triazin-2-yl]amino}butan-1-ol; 3-({2-[(4-{[6-(cyclohexylmethoxy)-9h- |
| | purin-2-yl]amino}phenyl)sulfonyl]ethyl}amino)propan-1-ol; 6- |
| | cyclohexylmethyloxy-5-nitroso-pyrimidine-2,4-diamine; 1-methyl-8- |
| | (phenylamino)-4,5-dihydro-1H-pyrazolo[4,3-h]quinazoline-3-carboxylic acid; 6- |
| | bromo-13-thia-2,4,8,12,19-pentaazatricyclo[12.3.1.1~3,7~]nonadeca- |
| | 1(18),3(19),4,6,14,16-hexaene 13,13-dioxide; (2R)-2-({9-(1-methylethyl)-6-[(4- |
| | pyridin-2-ylbenzyl)amino]-9H-purin-2-yl}amino)butan-1-ol; 1-[4- |
| | (aminosulfonyl)phenyl]-1,6-dihydropyrazolo[3,4-e]indazole-3-carboxamide; 5- |
| | (2,3-dichlorophenyl)-N-(pyridin-4-ylmethyl)pyrazolo[1,5-a]pyrimidin-7-amine; |
| | 6-(2-fluorophenyl)-N-(pyridin-3-ylmethyl)imidazo[1,2-a]pyrazin-8-amine; 3- |
| | methyl-N-(pyridin-4-ylmethyl)imidazo[1,2-a]pyrazin-8-amine; 5-(2- |
| | fluorophenyl)-N-(pyridin-4-ylmethyl)pyrazolo[1,5-a]pyrimidin-7-amine; 3- |
| | bromo-5-phenyl-N-(pyridin-3-ylmethyl)pyrazolo[1,5-a]pyrimidin-7-amine; 3- |
| | bromo-5-phenyl-N-(pyrimidin-5-ylmethyl)pyrazolo[1,5-a]pyridin-7-amine; 3- |
| | bromo-6-phenyl-N-(pyrimidin-5-ylmethyl)imidazo[1,2-a]pyridin-8-amine; N- |
| | ((2-aminopyrimidin-5-yl)methyl)-5-(2,6-difluorophenyl)-3-ethylpyrazolo[1,5- |
| | a]pyrimidin-7-amine; 3-cyclopropyl-5-phenyl-N-(pyridin-3- |
| | ylmethyl)pyrazolo[1,5-a]pyrimidin-7-amine; 4-{[4-amino-6- |
| | (cyclohexylmethoxy)-5-nitrosopyrimidin-2-yl]amino}benzamide; 4-[(5- |
| | isopropyl-1,3-thiazol-2-yl)amino]benzenesulfonamide; N-(5-Isopropyl-thiazol-2- |
| | YL)-2-pyridin-3-YL-acetamide; Variolin B; N(6)-dimethylallyladenine; |
| | Bosutinib, Milciclib, SNS-032, CVT-313, Isoindirubin, Amygdalin, Zotiraciclib |
| | citrate, Milciclib maleate, Indirubin |
| MAPK1 (mitogen- | Ulixertinib, Arsenic trioxide, Phosphonothreonine, Purvalanol, Seliciclib, |
| activated protein | Perifosine, Isoprenaline, N,N-dimethyl-4-(4-phenyl-1h-pyrazol-3-yl)-1h-pyrrole- |
| kinase 1) | 2-carboxamide; N-benzyl-4-[4-(3-chlorophenyl)-1h-pyrazol-3-yl]-1h-pyrrole-2- |
| | carboxamide; (S)-N-(1-(3-chloro-4-fluorophenyl)-2-hydroxyethyl)-4-(4-(3- |
| | chlorophenyl)-1h-pyrazol-3-yl)-1h-pyrrole-2-carboxamide; (3R,5Z,8S,9S,11E)- |
| | 8,9,16-trihydroxy-14-methoxy-3-methyl-3,4,9,10-tetrahydro-1h-2- |
| | benzoxacyclotetradecine-1,7(8h)-dione; 5-(2-phenylpyrazolo[1,5-a]pyridin-3-yl)- |
| | 1h-pyrazolo[3,4-c]pyridazin-3-amine; (1aR,8S,13S,14S,15aR)-5,13,14- |
| | trihydroxy-3-methoxy-8-methyl-8,9,13,14,15,15a-hexahydro-6H- |
| | oxireno[k][2]benzoxacyclotetradecine-6,12(1aH)-dione; Olomoucine; [4-({5- |
| | (aminocarbonyl)-4-[(3-methylphenyl)amino]pyrimidin-2-yl}amino)phenyl]acetic |
| | acid; 4-[4-(4-fluorophenyl)-2-[4-[(r)-methylsulfinyl]phenyl]-1h-imidazol-5- |
| | yl]pyridine; SB220025; Turpentine |
| GSK3B (Glycogen | Lithium cation; 3-[3-(2,3-Dihydroxy-Propylamino)-Phenyl]-4-(5-Fluoro-1- |
| Synthase Kinase 3 | Methyl-1h-Indol-3-Yl)-Pyrrole-2,5-Dione; SB-409513; AR-AO-14418; |
| Beta) | Staurosporine; Indirubin-3′-monoxime; Alsterpaullone; |
| | Phosphoaminophosphonic Acid-Adenylate Ester; 2-(1,3-benzodioxol-5-yl)-5-[(3- |
| | fluoro-4-methoxybenzyl)sulfanyl]-1,3,4-oxadiazole; 5-[1-(4-methoxyphenyl)- |
| | 1H-benzimidazol-6-yl]-1,3,4-oxadiazole-2(3H)-thione; (7S)-2-(2- |
| | aminopyrimidin-4-yl)-7-(2-fluoroethyl)-1,5,6,7-tetrahydro-4H-pyrrolo[3,2- |
| | c]pyridin-4-one; 6-bromoindirubin-3′-oxime; N-[2-(5-methyl-4H-1,2,4-triazol-3- |
| | yl)phenyl]-7H-pyrrolo[2,3-d]pyrimidin-4-amine; 5-(5-chloro-7H-pyrrolo[2,3- |
| | d]pyrimidin-4-yl)-4,5,6,7-tetrahydro-1H-imidazo[4,5-c]pyridine; 3-({[(3S)-3,4- |
| | dihydroxybutyl]oxy}amino)-1H,2′H-2,3′-biindol-2′-one; N-[(1S)-2-amino-1- |
| | phenylethyl]-5-(1H-pyrrolo[2,3-b]pyridin-4-yl)thiophene-2-carboxamide; 4-(4- |
| | chlorophenyl)-4-[4-(1h-pyrazol-4-yl)phenyl]piperidine; isoquinoline-5-sulfonic |
| | acid (2-(2-(4-chlorobenzyloxy)ethylamino)ethyl)amide; (2S)-1-(1H-indol-3-yl)- |
| | 3-{[5-(3-methyl-1h-indazol-5-yl)pyridin-3-yl]oxy}propan-2-amine; Tideglusib; |
| | Fostamatinib; Lithium citrate; Lithium succinate; Lithium carbonate |
| CSNK2A1 (Casein | Silmitasertib, Benzamidine; Phosphoaminophosphonic Acid-Adenylate Ester; |
| kinase II subunit | Tetrabromo-2-Benzotriazole; Resveratrol; s-methyl-4,5,6,7-tetrabromo- |
| alpha) | benzimidazole; Emodin; 3,8-dibromo-7-hydroxy-4-methyl-2h-chromen-2-one; |
| | 1,8-Di-Hydroxy-4-Nitro-Anthraquinone; (5-hydroxyindolo[1,2-a]quinazolin-7- |
| | yl)acetic acid; dimethyl-(4,5,6,7-tetrabromo-1h-benzoimidazol-2-yl)-amine; |
| | N1,N2-ethylene-2-methylamino-4,5,6,7-tetrabromo-benzimidazole; 1,8-Di- |
| | Hydroxy-4-Nitro-Xanthen-9-One; 5,8-Di-Amino-1,4-Dihydroxy-Anthraquinone; |
| | 19-(cyclopropylamino)-4,6,7,15-tetrahydro-5H-16,1-(azenometheno)-10,14- |
| | (metheno)pyrazolo[4,3-o][1,3,9]triazacyclohexadecin-8(9H)-one; N,N′- |
| | diphenylpyrazolo[1,5-a][1,3,5]triazine-2,4-diamine; 4-(2-(1h-imidazol-4- |
| | yl)ethylamino)-2-(phenylamino)pyrazolo[1,5-a][1,3,5]triazine-8-carbonitrile; 2- |
| | (cyclohexylmethylamino)-4-(phenylamino)pyrazolo[1,5-a][1,3,5]triazine-8- |
| | carbonitrile; 2-(4-chlorobenzylamino)-4-(phenylamino)pyrazolo[1,5- |
| | a][1,3,5]triazine-8-carbonitrile; 2-(4-ethylpiperazin-1-yl)-4- |
| | (phenylamino)pyrazolo[1,5-a][1,3,5]triazine-8-carbonitrile; N-(3-(8-cyano-4- |
| | (phenylamino)pyrazolo[1,5-a][1,3,5]triazin-2-ylamino)phenyl)acetamide; |
| | Dichlororibofuranosylbenzimidazole; Quinalizarin; Ellagic acid; ATP; |
| | Quercetin; Fostamatinib |

1. A method of determining a Crohn's Disease (CD) subtype status in a subject having CD, wherein the status comprises distinguishing a CD PBmucosal (CD-PBmu) subtype from a non-CD-PBmu subtype, the method comprising:
detecting expression of one or more genes from Tables 1A-1B in a biological sample from the subject to obtain an expression profile comprising the expression levels of each of the one or more genes in the biological sample, and
determining the CD subtype status of the subject based upon the expression profile,
wherein an increased level of expression in the one or more genes in the biological sample as compared to a reference expression profile indicates status of CD-PBmu subtype as distinguished from a non-CD-PBmu subtype.

2. A method of selecting a treatment for a subject having a Crohn's Disease (CD) PBmucosal (CD-PBmu) subtype, the method comprising:
(a) determining a level of expression of one or more genes from Tables 1A-1B in a biological sample obtained from the subject having CD;
(b) detecting an expression profile comprising an increase in the level of expression of the one or more genes in the biological sample, relative to a reference expression profile; and
(c) identifying the subject as a candidate for treatment of Crohn's Disease based upon the expression profile that is detected in (b). The method of claim 1 or claim 2, wherein the one or more genes comprises (a) ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, MIR155HG, or PLA2G2A or a combination thereof, and/or (b) ADH4, ALG1L, BCDIN3D, C1orf106, C2, CCDC144NL, CEACAM5, CTAGE8, DDX11L2, DPPA4, DUSP19, FGB, GP2, GYPE, HSD3B7, HUNK, JAM2, KCNE3, KRT42P, LYZ, MLLT10P1, NAP1L6, NEURL3, NPIPB9, PANK1, PKIB, RHOU, RPSAP9, SHCBP1, SIGLEC8, SLC15A2, SLC25A34, SLC6A20, SLC9B1, SYNPO2L, TDGF1, ZNF491, ZNF620, ZNF69, CXCL16, CD68, or CD300E, or a combination thereof.

3. The method of claim 1, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, MIR155HG, or UBD, or a combination thereof.

4. The method of any previous claim, wherein the increase in the level of expression of the one or more genes in the biological sample is at least 2-fold greater than in the reference expression profile.

5. The method of claim 1, wherein the reference expression profile comprises expression levels of the one or more genes of one or more subjects that do not have CD.

6. The method of claim 1, wherein determining a level of expression of one or more genes comprises utilizing an assay selected from the group consisting of an RNA sequencing method, a microarray method, and quantitative polymerase chain reaction (qPCR).

7. The method of claim 1, wherein determining a level of expression of one or more genes comprises:
(a) contacting the biological sample with a nucleic acid primer and/or detectable nucleic acid probe; and
(b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

8. The method of claim 1, wherein the CD is associated with perianal disease/fistula.

9. The method of claim 1, wherein the CD is associated with stricturing disease.

10. The method of claim 1, wherein the CD is associated with recurrence.

11. The method of claim 1, wherein the CD is associated with increased immune reactivity to a microbial antigen.

12. The method of claim 1, wherein the expression of at least one of the one or more genes in the biological sample is at least 2-fold greater than in the reference expression profile.

13. The method of claim 1, wherein the reference expression profile comprises expression levels of the one or more genes of one or more subjects who do not have IBD or have a PBT subtype of CD.

14. The method of claim 1, wherein the reference expression profile is stored in a database.

15. The method of claim 1, further comprising treating the subject with a therapeutic agent.

16. A method of treating a subject having a Crohn's Disease (CD) PBmucosal (CD-PBmu) subtype, the method comprising:
(a) determining a level of expression of one or more genes from Tables 1A-1B in a biological sample obtained from the subject having CD;
(b) detecting an expression profile comprising an increase in the level of expression of the one or more genes in the biological sample, relative to a reference expression profile; and
(c) administering to the subject a therapeutic agent against Crohn's Disease based upon the expression profile that is detected in (b).

17. The method of claim 16, wherein the therapeutic agent comprises a therapeutic of Table 20B; a protein, peptide, nucleic acid, or compound that targets a molecule of Tables 14, 15, 17A-17B, or 20A; or a compound that targets a molecule in a pathway of one or more genes of Table 17B; or any combination thereof.

18. The method of claim 16, wherein the therapeutic agent comprises a modulator of miR-155.

19. The method of claim 18, wherein the miR-155 modulator comprises an inhibitor of miR-155.

20. The method of claim 18, wherein the miR-155 modulator comprises one or more oligonucleotides of Tables 3-12.

21.-41. (canceled)

42. A method for processing or analyzing a biological sample from a subject, comprising:
(a) obtaining the biological sample comprising gene expression products, wherein the subject has or is suspected of having Crohn's Disease (CD);
(b) subjecting the biological sample to an assay by sequencing, array hybridization, and/or nucleic acid amplification to yield a data set including data corresponding to gene expression product levels;
(c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and
(d) electronically outputting a report that identifies the classification of the biological sample as positive or negative for the CD subtype.
